(19) United States
(12) Patent Application Publication
(10) Pub. No.: US 2026/0273072 A1
(43) Pub. Date: Sep. 17, 2026

(54) NOVEL PLK1 DEGRADATION INDUCING COMPOUND

(71) Applicant: UPPTHERA, INC., Incheon (KR)

(72) Inventors: Soo Hee Ryu, Incheon (KR); Seong Hoon Kim, Incheon (KR); Keum Young Kang, Incheon (KR); Solhee Noh, Incheon (KR); Jun Kyu Lee, Incheon (KR)

(73) Assignee: UPPTHERA, INC., Incheon (KR)

(21) Appl. No.: 18/994,994

(22) PCT Filed: Feb. 2, 2024
(86) PCT No.: PCT/KR2024/001626

§ 371 Date: Jan. 15, 2025

(30) Foreign Application Priority Data
Feb. 2, 2023 (KR) 10-2023-0014348
Sep. 4, 2023 (KR) 10-2023-0116774

(51) Int. Cl. A61K 47/55; A61K 47/54; A61P 35/00

(52) U.S. Cl. CPC A61K 47/55; A61K 47/545; A61P 35/00

(57) ABSTRACT

The present disclosure relates to a novel PLK1 degradation inducing compound, a method for preparing the same, and the use thereof. The compounds of the present disclosure exhibit an effect of inducing PLK1 degradation. Therefore, the compounds of the present disclosure may be effectively utilized for preventing or treating PLK1-related diseases.

### TECHNICAL FIELD

The present disclosure relates to a novel PLK1 degradation inducing compound, a method for preparing the same, and the use thereof. It can specifically act on abnormal cells, etc. and can be usefully used in the treatment of various diseases through efficient degradation of PLK1.

### BACKGROUND ART

Polo-like kinase 1 (PLK1) is a serine/threonine kinase involved in the conversion of G2/M phase during cell growth and division. PLK1 is expressed and activated in a pulse form from the S phase to the G2/M phase, and rapidly degrades as mitosis ends.

PLK1 is overexpressed in various carcinomas such as colon cancer, lung cancer, bladder cancer, and melanoma, etc., and cancer cells overexpressing PLK1 tend to show resistance to various types of anticancer drugs. As the PLK1 dependence in various carcinomas was revealed as described above, there have been attempts to develop PLK1 inhibitor compounds such as volasertib (also known as BI6727), etc.

However, the conventional PLK1 inhibitors do not sufficiently inhibit PLK1 activity at concentrations that are clinically safe. Thus, there is a problem that even if the cell cycle of cancer cells is temporarily delayed, some cancer cells eventually restart the cell cycle, which may not obtain sufficient clinical effects (see Gheghiani et al., Cell Reports, 2017, 19: 2060, etc.). In fact, many pharmaceutical companies such as Boehringer Ingelheim, GlaxoSmithKline, etc., have attempted to develop small-molecular compound-based PLK1 inhibitors, but most of them have failed or stopped in the clinical trial stage, and thus there are no commercially available PLK1 inhibitors to date. It shows that pharmacological mechanism that follows the method of inhibiting enzyme activity by binding to the active site of PLK1 like the small molecule compound inhibitors is not sufficiently effective in the development of new drugs intended to derive anticancer effects by inhibiting PLK1 activity of cancer cells.

Recently, a proteolysis targeting chimera (PROTAC) has been proposed as a small molecule-based platform technology capable of inducing proteolysis of a target protein in the body. The PROTAC is a bifunctional compound in which a ligand molecule that binds to disease-related target protein and an E3 ubiquitin ligase binding moiety are linked by a chemical linker. Theoretically, the PROTAC compound is capable of inducing degradation of the target protein by placing the disease-related target protein near the E3 ubiquitin ligase. Based on this new mechanism different from the existing inhibitors, a lot of PROTAC compounds have been developed as therapeutic agents for cancer and inflammatory diseases, etc., and being studied with various extensibility (e.g. as payloads of ADC (Antibody-Drug Conjugates)). However, it does not show activity in all ranges of binding moieties or linkers, and in order for PROTAC to exhibit the desired level of efficacy, it is known through several studies that each binding moiety and linker must have an appropriately linked structure (see US2020-0325130A). In particular, in the case of the CRBN (Cereblon) E3 ligase targeting moiety, depending on the type of the binding moiety or the structure of the compound linked thereto, there is a risk of degrading CRBN neo-substrate (GSPT1, IKZF1/3, etc.) or showing off-target toxicity accordingly. Therefore, it is important to select appropriate binding moieties and optimize the structure of the entire compound so as not to exhibit unexpected toxicity during PROTAC drug development.

In the case of the PROTAC compound using PLK1 as a target protein, Chinese Patent Laid-Open No. 106543185 A discloses some bifunctional compounds in which a volasertib derivative compound and a binding moiety for the E3 ubiquitin ligase CRBN are linked by a chemical linker. However, the related art document merely describes some limited forms of synthesis examples of PROTAC compounds, wherein in general, the target degradation activity and selectivity of PROTAC may vary significantly depending on selection of the target protein moiety, the E3 ubiquitin ligase binding moiety, and the like (see Burslem and Crews, Chemical Reviews, 2017, 117(17): 11269, etc.).

Further, the PROTAC compound described in the above-described document is characterized by a compound that simultaneously degrades PLK1 and BRD4, and degrade various proteins such as other PLK family proteins and BRD4, etc., which may cause side effects due to off-target toxicities at the time of drug development. In particular, it is known that strong inhibition of BRD4 activity inevitably accompanies on-target toxicity such as blood toxicity and gastrointestinal toxicity along with pharmacological effects. Therefore, the PROTAC compound described in the above document would expect to face greater clinical side effects as more BRD4 protein gets degraded (see Bolden et al. Cell Reports, 2014, 8(6): 1919).

Moreover, according to the document published by the inventors of the above document (see Mu et al. BBRC, 2020, 521(4): 833), it can be confirmed that the PROTAC compound, which simultaneously degrades PLK1 and BRD4, has much stronger BRD4 degradation ability than PLK1 degradation ability at the cellular level, and the cell cycle thereof almost stops in the G1 phase, etc., that is, the PROTAC compound actually acts only as a BRD4 inhibitor regardless of the way that the conventional PLK1 inhibitors exert pharmacological effects.

Therefore, there is an unsatisfied demand for effective PLK1 degradation inducing compound with no or minimal side effects. (e.g. off-target toxicity)

### DISCLOSURE OF INVENTION

### Technical Problem

An object of the present disclosure is to provide novel PLK1 degradation inducing compounds.

Another object of the present disclosure is to provide a method for preparing the compounds.

Still another object of the present disclosure is to provide a use of the compounds.

### Solution to Problem

In order to achieve the above-described objects, the present inventors made efforts to study, and as a result, found that novel PROTAC compounds of the present invention specifically act on abnormal cells overexpressing PLK1 through appropriate structural combination and optimization of E3 Ligase binder, Target binding moiety, and Linker to induce effective PLK1 degradation and minimize side effects, and completed the present invention.

### Selective PLK1 Degradation Inducing Compounds

The present disclosure provides novel compounds that induce effective polo-like kinase 1 (PLK1) degradation. Specifically, the present disclosure provides a bifunctional compound in which a PLK1 binding moiety and an E3 ubiquitin ligase-binding moiety are linked by a chemical linker.

In one general aspect, there is provided a compound represented by the following Formula I, a stereoisomer thereof or a pharmaceutically acceptable salt thereof:

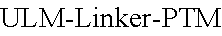

in the Formula I above,
ULM is a moiety represented by the following Formula 1;

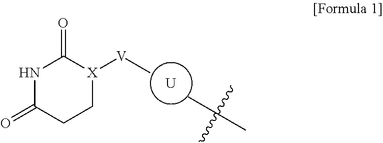

PTM is a moiety represented by the following Formula 2;

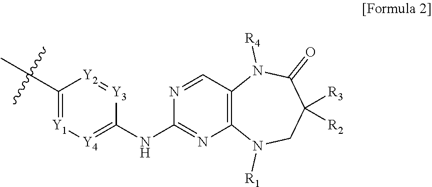

Linker is a group that chemically links ULM and PTM;
X is CH or N;
V is —NRᵥ— or a single bond;
Rᵥ is —H or —CH₃;
ring U is phenyl or 5- to 6-membered heteroaryl {wherein at least one H of the phenyl or 5- to 6-membered heteroaryl ring may be substituted with R_{U}};
R_{U} is —C₁₋₃alkyl, —C₁₋₃hydroxyalkyl, —C₁₋₃aminoalkyl, —C₁₋₃haloalkyl, —C₁₋₃alkoxy, —CN or -halo;
Y₁ is CR_{Y} or N;
Y₂ to Y₄ are each independently CR_{Y};
R_{Y} is —H, —C₁₋₃alkyl, —C₁₋₃alkoxy or -halo;
R₁ is —C₁₋₄alkyl or 5- to 6-membered cycloalkyl;
R₂ and R₃ are each independently —H, —C₁₋₃alkyl or -halo; and
R₄ is —C₁₋₃alkyl.

In one embodiment of the present disclosure,
ULM is a moiety represented by following Formula 1-1, Formula 1-2 or Formula 1-3;

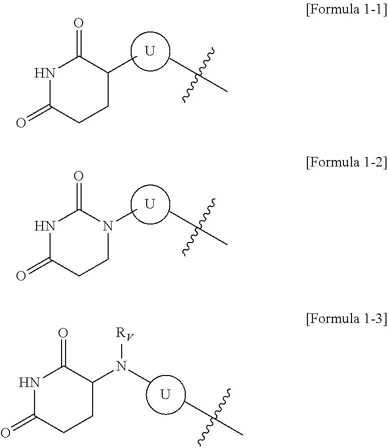

Rᵥ is —H or —CH₃,
ring U is phenyl or pyridinyl {wherein at least one H of the phenyl or pyridinyl ring may be substituted with R_{U}}; and
R_{U} is —C₁₋₃alkyl, —C₁₋₃alkoxy, —CN or -halo.

In one embodiment of the present disclosure,
ULM is

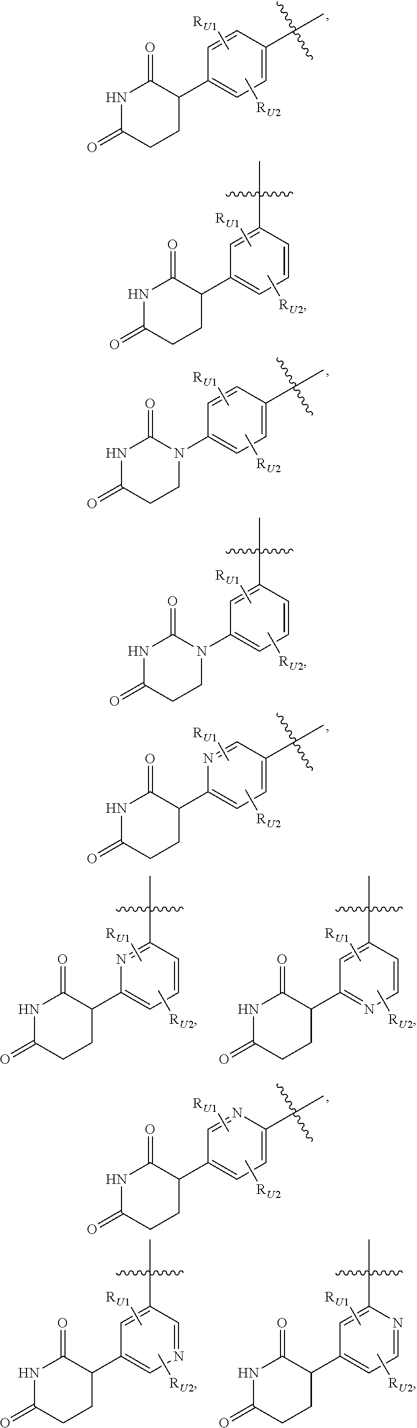

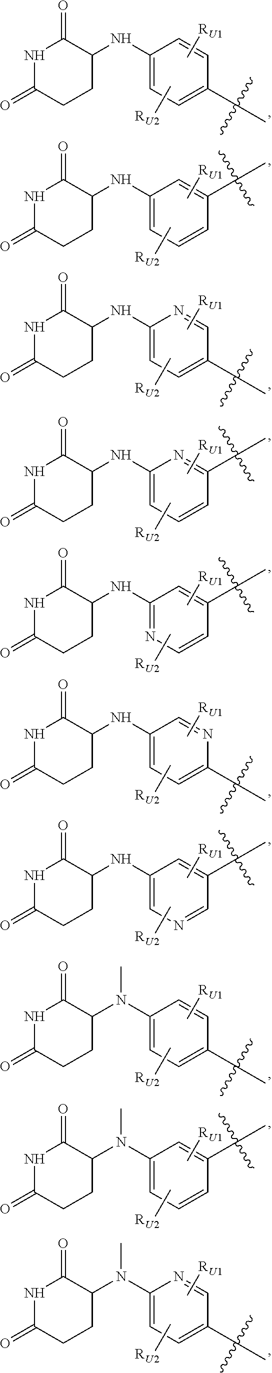

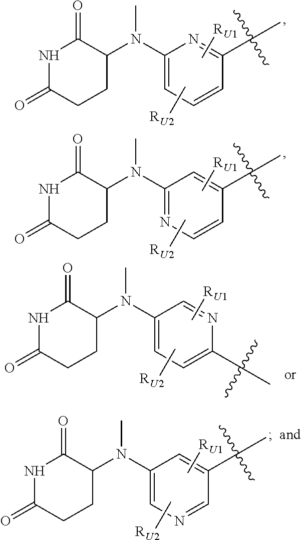

R_{U1} and R_{U2} are each independently —C₁₋₃alkyl, —C₁₋₃alkoxy, —CN or -halo.

In one embodiment of the present disclosure,
ULM is

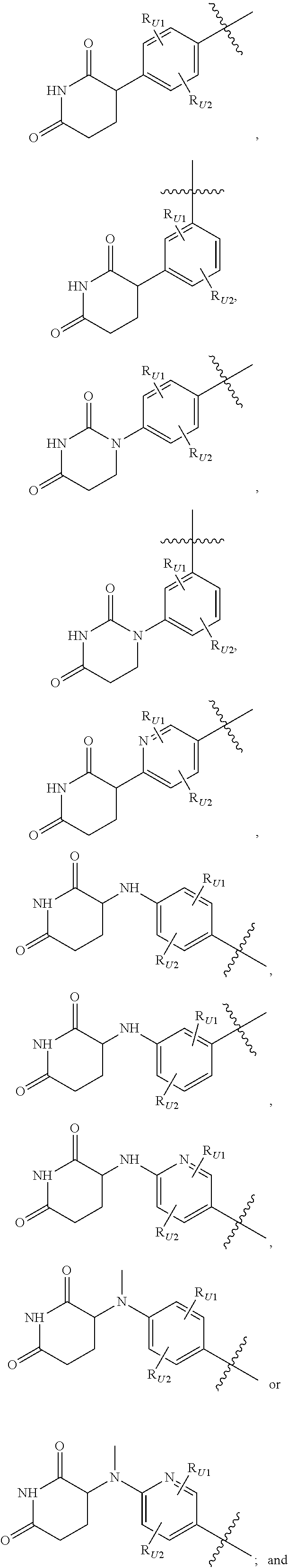

R_{U1} and R_{U2} are each independently —C₁₋₃alkyl, —C₁₋₃alkoxy, —CN or -halo.

In one embodiment,
PTM is;

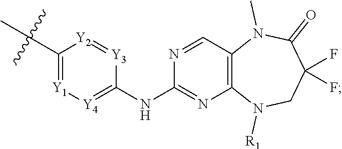

Y₁ is CR_{Y} or N;
Y₂ to Y₄ are each independently CR_{Y};
R_{Y} is —H, —C₁₋₃alkoxy or -halo;
R₁ is —C₁₋₄alkyl or 5- to 6-membered cycloalkyl. Preferably, but not limited thereto, R₁ may be methyl, ethyl, isopropyl or cyclopentyl.

In one embodiment of the present disclosure,
Linker is -L_{U}-L₁-L₂-Lₚ-;
L_{U} is heterocycloalkyl {wherein the heterocycloalkyl contains at least one N atom in the ring, and at least one H of the heterocycloalkyl ring may be substituted with —C₁₋₄ alkyl, —C₁₋₄alkoxy or -halo};
L₁ is —(CH₂)_{X1}—, —(CH₂)_{X2}—NH—(CH₂)_{X3}—, —(CH₂)_{X2}—N(C₁₋₄alkyl)-(CH₂)_{X3}— or a single bond {wherein the X1 to X3 are each independently 0, 1, 2, 3 or 4};
L₂ is cycloalkyl or heterocycloalkyl {wherein the heterocycloalkyl contains at least one N atom in the ring, and at least one H of the cycloalkyl or heterocycloalkyl ring may be substituted with —C₁₋₄alkyl, —C₁₋₄alkoxy or -halo}; and
Lₚ is —NH—C(═O)— or —(CH₂)p-heteroaryl {wherein —(C═O)— of the —NH—C(═O)— or heteroaryl of the —(CH₂)p-heteroaryl is linked with PTM, and the heteroaryl contains N and O atoms in the ring, and the p is 0, 1, 2 or 3}.

In one embodiment of the present disclosure,
L_{U} is 4- to 12-membered heterocycloalkyl {wherein the 4- to 12-membered heterocycloalkyl is single ring, bridged bicyclic ring or spiro ring, the 4- to 12-membered heterocycloalkyl contains at least one N atom in the ring, the N atom is directly linked with ULM, and at least one H of the 4- to 12-membered heterocycloalkyl ring may be substituted with —C₁₋₄alkyl or -halo};
L₁ is —(CH₂)_{X1}—, —(CH₂)_{X2}—N(C₁₋₄alkyl)-(CH₂)_{X3}— or a single bond {wherein the X1 to X3 are each independently 0, 1, 2 or 3}
L₂ is 4- to 6-membered cycloalkyl or 4- to 12-membered heterocycloalkyl{wherein the 4- to 12-membered heterocycloalkyl is single ring, bridged bicyclic ring or spiro ring, the 4- to 12-membered heterocycloalkyl contains at least one N atom in the ring, and at least one H of the 4- to 6-membered cycloalkyl or 4- to 12-membered heterocycloalkyl ring may be substituted with —C₁₋₄alkyl or -halo}; and
Lₚ is —NH—C(═O)— or —(CH₂)p-1,3,4-oxadiazol {wherein —(C═O)— of the —NH—C(═O)— or 1,3,4-oxadiazol of the —(CH₂)p-1,3,4-oxadiazol is linked with PTM, and the p is 0 or 1}.

Preferably, but not limited thereto, L_{U} may be

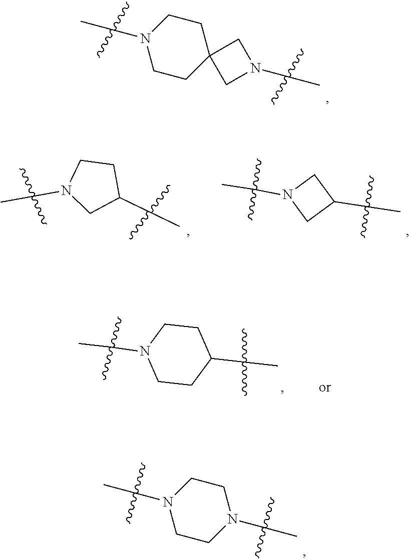 [SMILES: *N1CCC2(CC1)CN(*)C2 ; *C1CCN(*)C1 ; *C1CN(*)C1 ; *C1CCN(*)CC1 ; *N1CCN(*)CC1]

### and L₂ may be

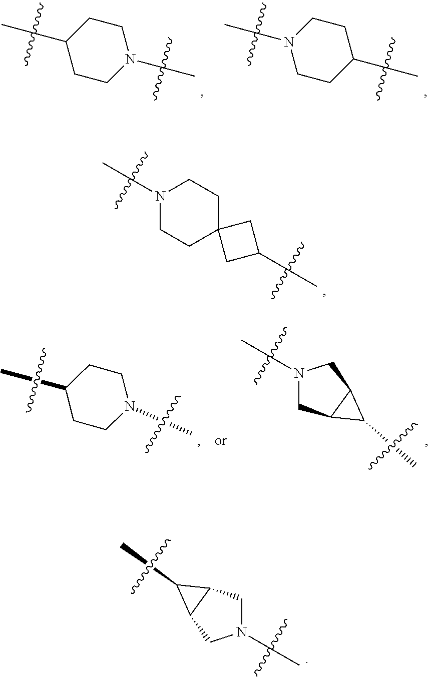

In a certain embodiment of the present disclosure, the compound represented by Formula I is a compound that is selected from the group consisting of Compound 1 to 77.

In the present disclosure, a pharmaceutically acceptable salt refers to any organic or inorganic acid addition salt with a concentration that is relatively non-toxic, is harmless, and has effective action to patients, wherein side effects caused by this salt does not deteriorate beneficial efficacy of the compound represented by Formula I. For example, the pharmaceutically acceptable salt may be an inorganic acid such as hydrochloric acid, phosphoric acid, sulfuric acid, nitric acid, or the like, or an organic acid such as methanesulfonic acid, p-toluenesulfonic acid, acetic acid, trifluoroacetic acid, maleic acid, succinic acid, oxalic acid, benzoic acid, tartaric acid, fumaric acid, manderic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid or hydroiodic acid, but is not limited thereto.

### Use of the Selective PLK1 Degradation Inducing Compounds

An embodiment of the present disclosure is a composition for inducing PLK1 degradation including a compound represented by Formula I or a pharmaceutically acceptable salt thereof. The Formula I is the same as defined above.

In the experimental examples of the present disclosure, it was confirmed that the compounds of the present disclosure effectively induce the protein degradation of PLK1.

The PLK1 degradation-inducing PROTAC compound of the present disclosure is capable of fundamentally degrading the target protein, PLK1 in view of the mechanism of action, thereby achieving an excellent PLK1 inhibitory effect as compared to the conventional PLK1 small molecule inhibitor that inhibits the simple activity of PLK1.

Accordingly, the composition including the compound represented by Formula I of the present disclosure or a pharmaceutically acceptable salt thereof may be effectively employed for selective degradation of PLK1.

An embodiment of the present disclosure is a composition for preventing or treating PLK1-related diseases including the compound represented by Formula I or the pharmaceutically acceptable salt thereof. Another embodiment of the present disclosure is a method for the prevention or treatment of PLK-related diseases comprising administering the composition to a subject in need thereof. The Formula I is the same as defined above.

In the present disclosure, the PLK1-related disease refers to any disease or condition capable of being treated, alleviated, delayed, inhibited or prevented from induction of degradation or inhibition of activity of PLKL. In an embodiment, the PLK1-related disease may be a cancer (malignant tumor), a benign tumor, a neurological disease, or other genetic or non-genetic diseases caused by excessive cell division.

The cancer includes all cancers capable of exhibiting prophylactic or therapeutic efficacy due to inhibition of PLK1 activity, and may be solid cancer or blood cancer. For example, the cancer may be one or more selected from the group consisting of squamous cell carcinoma, small cell lung cancer, non-small cell lung cancer, lung adenocarcinoma, lung squamous cell carcinoma, peritoneal cancer, skin cancer, skin or intraocular melanoma, rectal cancer, anal muscle cancer, esophageal cancer, small intestine cancer, endocrine cancer, parathyroid cancer, adrenal cancer, soft tissue sarcoma, urethral cancer, chronic or acute leukemia, lymphocytic lymphoma, hepatocellular carcinoma, gastrointestinal cancer, gastric cancer, pancreatic cancer, glioblastoma, cervical cancer, ovarian cancer, liver cancer, bladder cancer, liver tumor, breast cancer, colon cancer, colorectal cancer, endometrial or uterine cancer, salivary gland cancer, kidney cancer, prostate cancer, vulvar cancer, thyroid cancer, head and neck cancer, brain cancer, osteosarcoma, solid tumor, blood cancer, bone cancer, large cell lymphoma, adrenocorticoid tumor, t cell lymphoma/leukemia, neuroendocrine cancer, neuroendocrine tumor, cholangiocarcinoma, neuroblastoma, glioblastoma, glioma, and the like, but is not limited thereto. The cancer includes not only primary cancer but also metastatic cancer.

The benign tumors include all benign tumors capable of exhibiting prophylactic or therapeutic efficacy due to the inhibition of PLK1 activity, such as benign tumors in pre-cancer stages, and may be solid tumors or blood tumors. For example, the tumor may be one or more selected from the group consisting of Barrett's esophagus, colon adenoma and polyp, breast fibroadenoma and cyst, monoclonal gammopathy of undetermined significance (MGUS), monoclonal lymphocytosis, and the like, but is not limited thereto.

The neurological diseases include all neurological diseases capable of exhibiting prophylactic or therapeutic efficacy due to the inhibition of PLK1 activity, and specifically, may be one or more selected from the group consisting of central nervous system disease, neurodegenerative disease, Alzheimer's disease, Parkinson's disease, multiple sclerosis, Huntington's disease, senile dementia, epilepsy, Lou Gehrig, stroke, and nerve damage and axonal degeneration-related disorders following brain or spinal cord injury, but is not limited thereto.

The pharmaceutical composition of the present disclosure may further include one or more active ingredients exhibiting the same or similar medicinal effects in addition to the compound represented by Formula I above, or the pharmaceutically acceptable salt thereof.

An embodiment of the present disclosure is a method of degrading PLK1 by administering a compound represented by Formula I or a pharmaceutically acceptable salt thereof to mammals including humans.

Another embodiment of the present disclosure is a method of degrading PLK1 by administering the compound represented by Formula I or the pharmaceutically acceptable salt thereof to a sample in vitro. The sample may be a cell, a cell culture, a body fluid or tissue of a mammal including a human, but is not limited thereto.

### Advantageous Effects of Invention

The compounds of the present disclosure exhibit an effect of inducing PLK1 degradation. Therefore, the compounds of the present disclosure may be effectively utilized for preventing or treating PLK1-related diseases.

### BEST MODE FOR CARRYING OUT THE INVENTION

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

The present disclosure provides synthetic methods for Compound 1 to 77 shown in the table 1 below.

TABLE 1

| Compound | Structure |
|---|---|
| 1 | 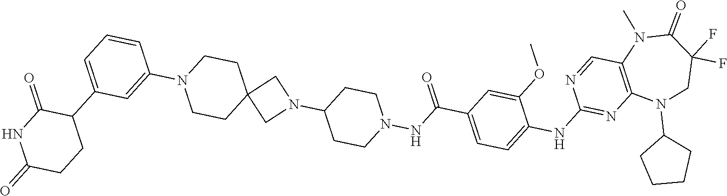 [SMILES: COc1cc(C(=O)NN2CCC(N3CC4(CCN(c5cccc(C6CCC(=O)NC6=O)c5)CC4)C3)CC2)ccc1Nc1ncc2c(n1)N(C1CCCC1)CC(F)(F)C(=O)N2C] |
| 2 | 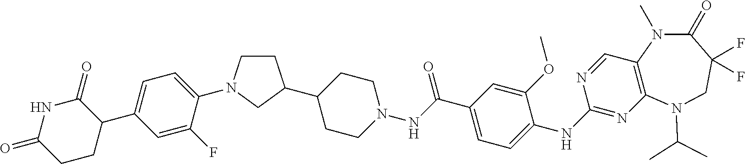 [SMILES: COc1cc(C(=O)NN2CCC(C3CCN(c4ccc(C5CCC(=O)NC5=O)cc4F)C3)CC2)ccc1Nc1ncc2c(n1)N(C(C)C)CC(F)(F)C(=O)N2C] |
| 3 | 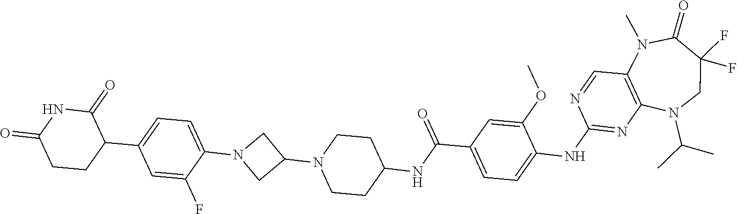 [SMILES: COc1cc(C(=O)NC2CCN(C3CN(c4ccc(C5CCC(=O)NC5=O)cc4F)C3)CC2)ccc1Nc1ncc2c(n1)N(C(C)C)CC(F)(F)C(=O)N2C] |
| 4 | 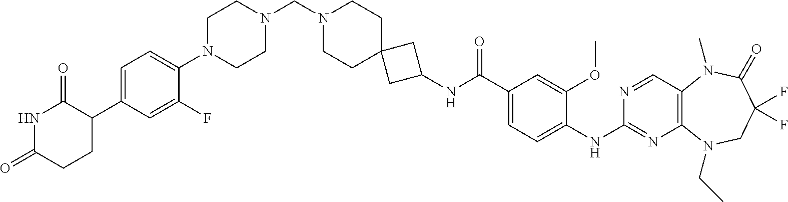 [SMILES: CCN1CC(F)(F)C(=O)N(C)c2cnc(Nc3ccc(C(=O)NC4CC5(CCN(CN6CCN(c7ccc(C8CCC(=O)NC8=O)cc7F)CC6)CC5)C4)cc3OC)nc21] |
| 5 | 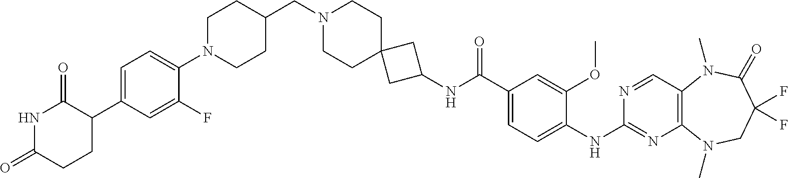 [SMILES: COc1cc(C(=O)NC2CC3(CCN(CC4CCN(c5ccc(C6CCC(=O)NC6=O)cc5F)CC4)CC3)C2)ccc1Nc1ncc2c(n1)N(C)CC(F)(F)C(=O)N2C] |
| 6 | 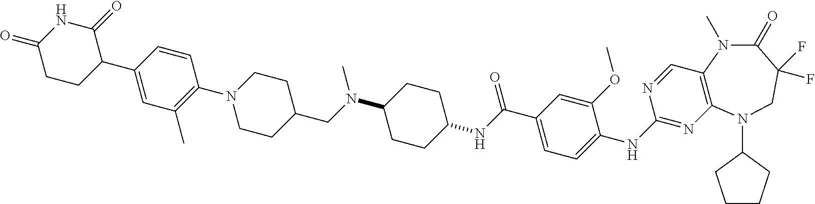 |
| 7 | 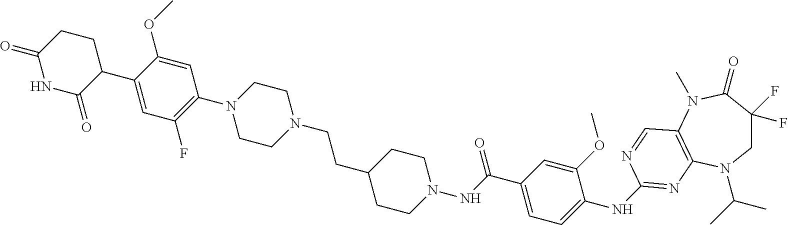 [SMILES: COc1cc(C(=O)NN2CCC(CCN3CCN(c4cc(OC)c(C5CCC(=O)NC5=O)cc4F)CC3)CC2)ccc1Nc1ncc2c(n1)N(C(C)C)CC(F)(F)C(=O)N2C] |
| 8 | 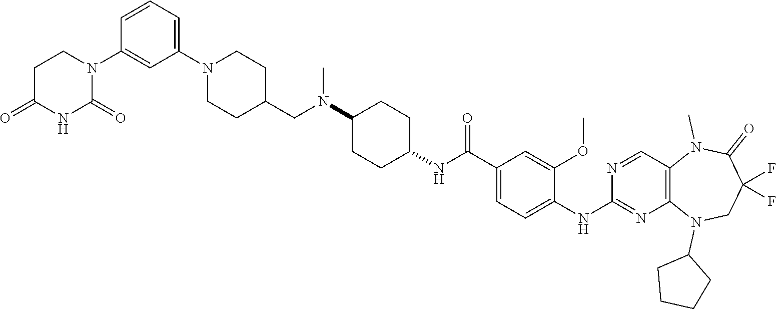 [SMILES: COc1cc(C(=O)N[C@H]2CC[C@H](N(C)CC3CCN(c4cccc(N5CCC(=O)NC5=O)c4)CC3)CC2)ccc1Nc1ncc2c(n1)N(C1CCCC1)CC(F)(F)C(=O)N2C] |
| 9 | 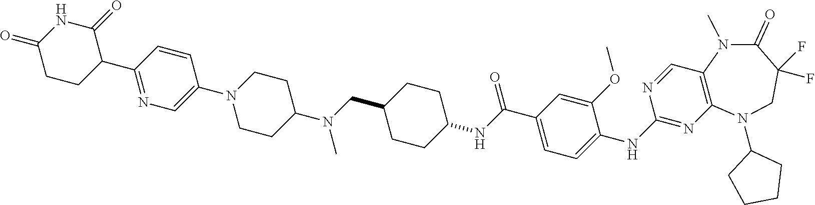 [SMILES: COc1cc(C(=O)N[C@H]2CC[C@H](CN(C)C3CCN(c4ccc(C5CCC(=O)NC5=O)nc4)CC3)CC2)ccc1Nc1ncc2c(n1)N(C1CCCC1)CC(F)(F)C(=O)N2C] |
| 10 | 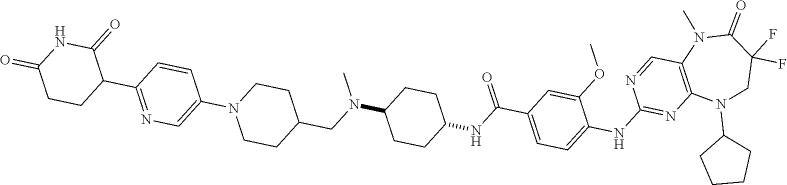 |
| 11 | 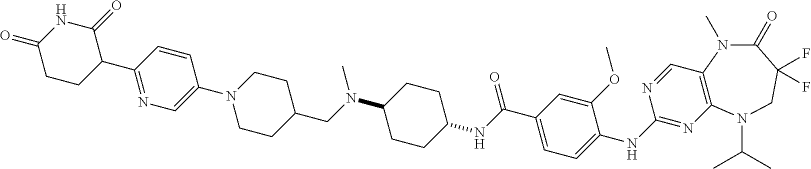 |
| 12 | 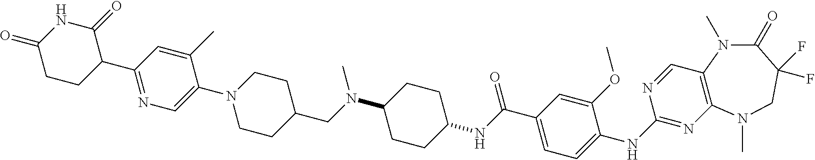 |
| 13 | 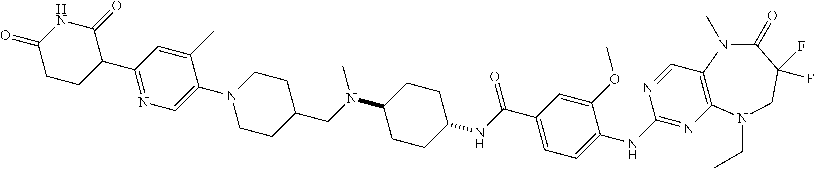 |
| 14 | 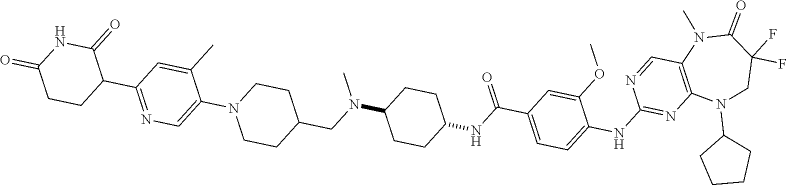 |
| 15 | 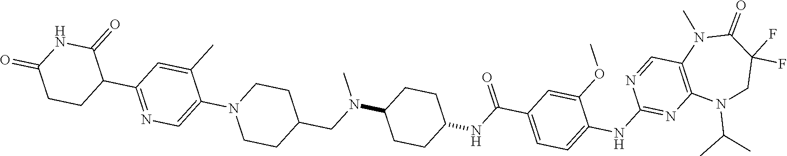 |
| 16 | 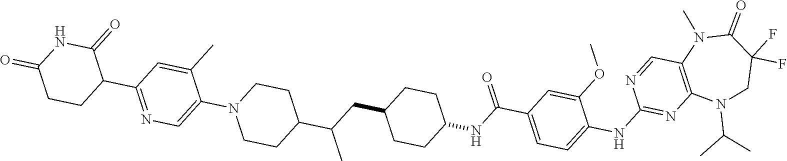 |
| 17 | 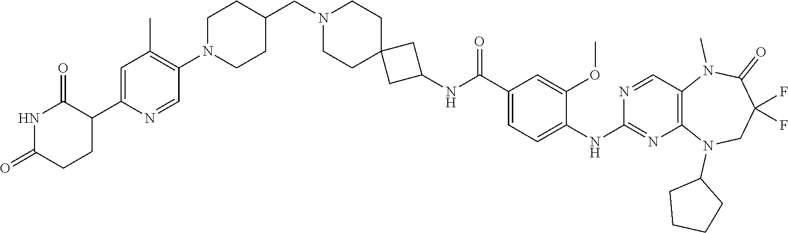 [SMILES: COc1cc(C(=O)NC2CC3(CCN(CC4CCN(c5cnc(C6CCC(=O)NC6=O)cc5C)CC4)CC3)C2)ccc1Nc1ncc2c(n1)N(C1CCCC1)CC(F)(F)C(=O)N2C] |
| 18 | 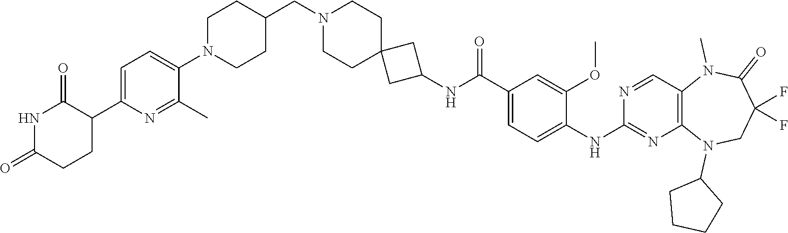 [SMILES: COc1cc(C(=O)NC2CC3(CCN(CC4CCN(c5ccc(C6CCC(=O)NC6=O)nc5C)CC4)CC3)C2)ccc1Nc1ncc2c(n1)N(C1CCCC1)CC(F)(F)C(=O)N2C] |
| 19 | 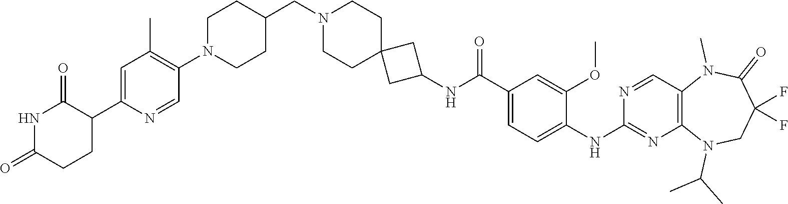 [SMILES: COc1cc(C(=O)NC2CC3(CCN(CC4CCN(c5cnc(C6CCC(=O)NC6=O)cc5C)CC4)CC3)C2)ccc1Nc1ncc2c(n1)N(C(C)C)CC(F)(F)C(=O)N2C] |
| 20 | 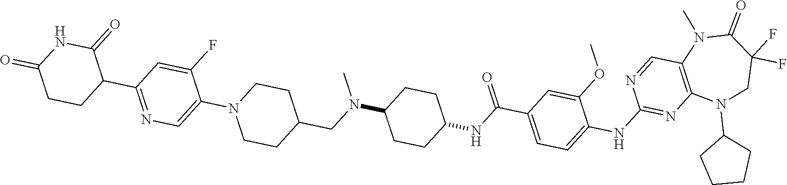 |
| 21 | 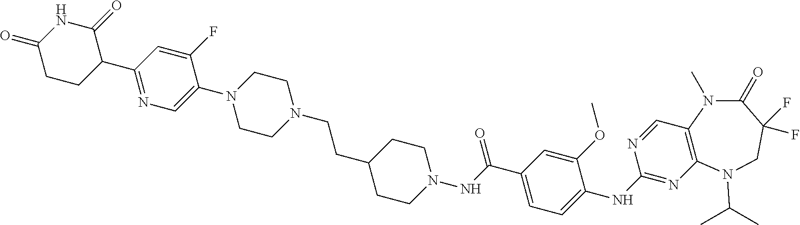 [SMILES: COc1cc(C(=O)NN2CCC(CCN3CCN(c4cnc(C5CCC(=O)NC5=O)cc4F)CC3)CC2)ccc1Nc1ncc2c(n1)N(C(C)C)CC(F)(F)C(=O)N2C] |
| 22 | 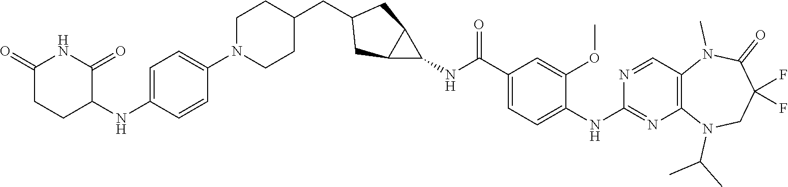 |
| 23 | 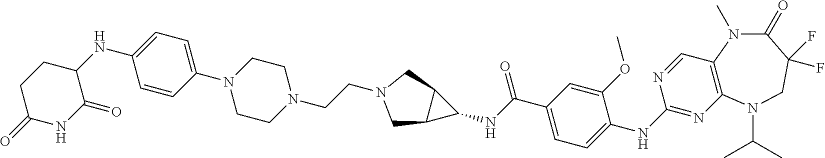 [SMILES: COc1cc(C(=O)N[C@H]2[C@@H]3CN(CCN4CCN(c5ccc(NC6CCC(=O)NC6=O)cc5)CC4)C[C@@H]32)ccc1Nc1ncc2c(n1)N(C(C)C)CC(F)(F)C(=O)N2C] |
| 24 | 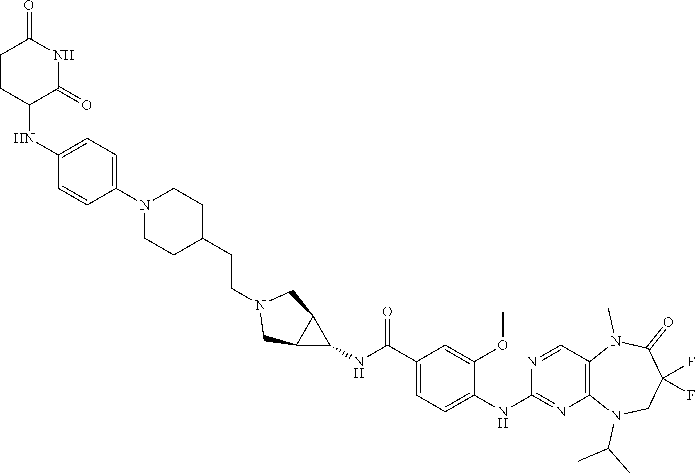 [SMILES: COc1cc(C(=O)N[C@H]2[C@@H]3CN(CCC4CCN(c5ccc(NC6CCC(=O)NC6=O)cc5)CC4)C[C@@H]32)ccc1Nc1ncc2c(n1)N(C(C)C)CC(F)(F)C(=O)N2C] |
| 25 | 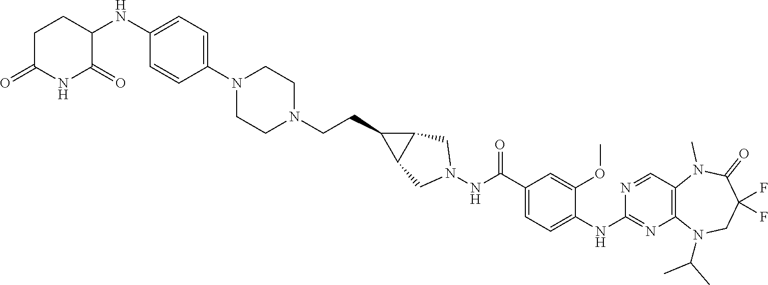 [SMILES: COc1cc(C(=O)NN2C[C@@H]3[C@@H](CCN4CCN(c5ccc(NC6CCC(=O)NC6=O)cc5)CC4)[C@@H]3C2)ccc1Nc1ncc2c(n1)N(C(C)C)CC(F)(F)C(=O)N2C] |
| 26 | 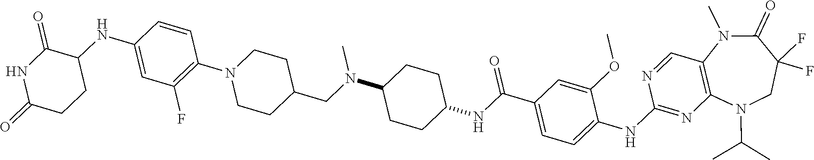 |
| 27 | 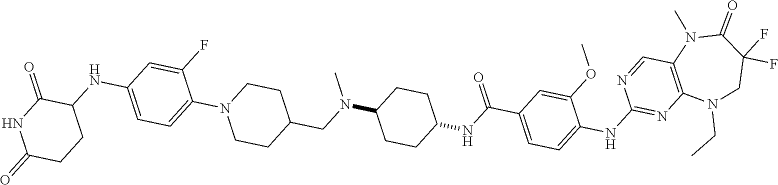 |
| 28 | 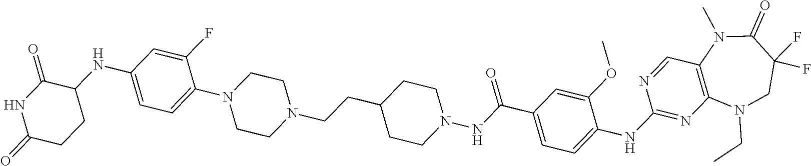 [SMILES: CCN1CC(F)(F)C(=O)N(C)c2cnc(Nc3ccc(C(=O)NN4CCC(CCN5CCN(c6ccc(NC7CCC(=O)NC7=O)cc6F)CC5)CC4)cc3OC)nc21] |
| 29 | 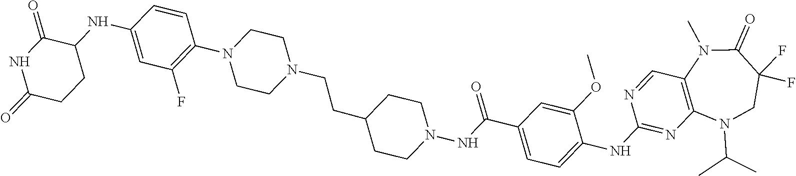 [SMILES: COc1cc(C(=O)NN2CCC(CCN3CCN(c4ccc(NC5CCC(=O)NC5=O)cc4F)CC3)CC2)ccc1Nc1ncc2c(n1)N(C(C)C)CC(F)(F)C(=O)N2C] |
| 30 | 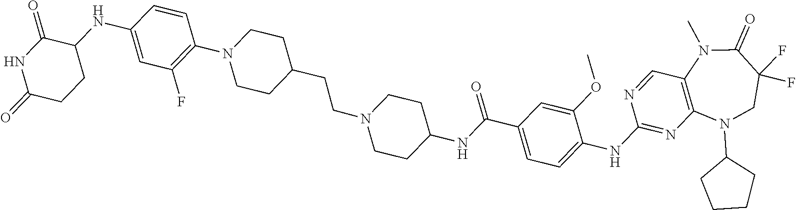 [SMILES: COc1cc(C(=O)NC2CCN(CCC3CCN(c4ccc(NC5CCC(=O)NC5=O)cc4F)CC3)CC2)ccc1Nc1ncc2c(n1)N(C1CCCC1)CC(F)(F)C(=O)N2C] |
| 31 | 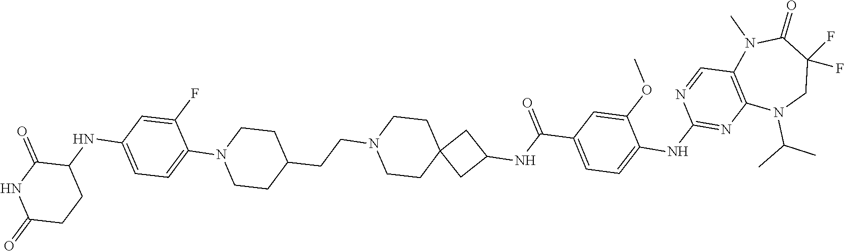 [SMILES: COc1cc(C(=O)NC2CC3(CCN(CCC4CCN(c5ccc(NC6CCC(=O)NC6=O)cc5F)CC4)CC3)C2)ccc1Nc1ncc2c(n1)N(C(C)C)CC(F)(F)C(=O)N2C] |
| 32 | 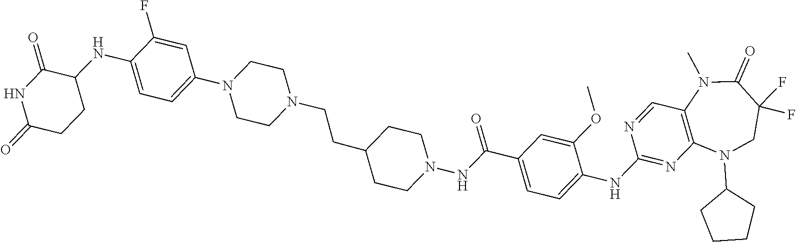 [SMILES: COc1cc(C(=O)NN2CCC(CCN3CCN(c4ccc(NC5CCC(=O)NC5=O)c(F)c4)CC3)CC2)ccc1Nc1ncc2c(n1)N(C1CCCC1)CC(F)(F)C(=O)N2C] |
| 33 | 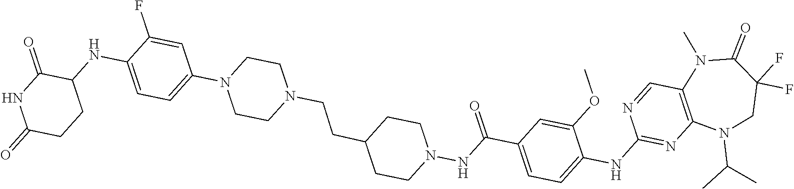 [SMILES: COc1cc(C(=O)NN2CCC(CCN3CCN(c4ccc(NC5CCC(=O)NC5=O)c(F)c4)CC3)CC2)ccc1Nc1ncc2c(n1)N(C(C)C)CC(F)(F)C(=O)N2C] |
| 34 | 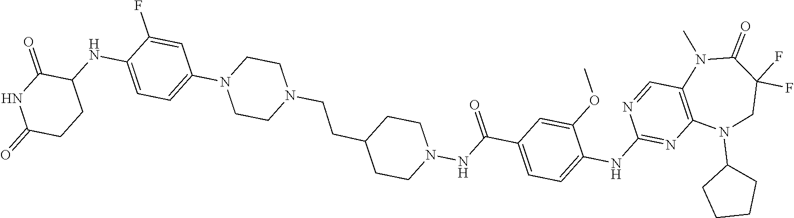 [SMILES: COc1cc(C(=O)NN2CCC(CCN3CCN(c4ccc(NC5CCC(=O)NC5=O)c(F)c4)CC3)CC2)ccc1Nc1ncc2c(n1)N(C1CCCC1)CC(F)(F)C(=O)N2C] |
| 35 | 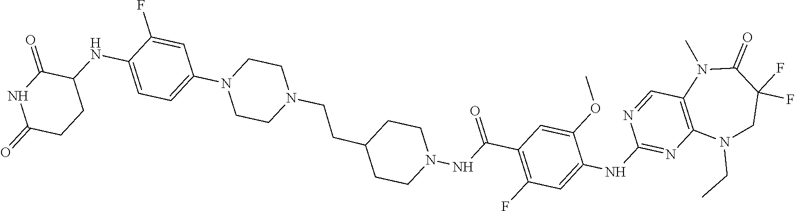 [SMILES: CCN1CC(F)(F)C(=O)N(C)c2cnc(Nc3cc(F)c(C(=O)NN4CCC(CCN5CCN(c6ccc(NC7CCC(=O)NC7=O)c(F)c6)CC5)CC4)cc3OC)nc21] |
| 36 | 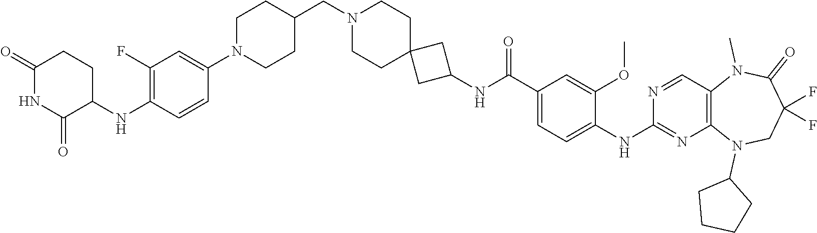 [SMILES: COc1cc(C(=O)NC2CC3(CCN(CC4CCN(c5ccc(NC6CCC(=O)NC6=O)c(F)c5)CC4)CC3)C2)ccc1Nc1ncc2c(n1)N(C1CCCC1)CC(F)(F)C(=O)N2C] |
| 37 | 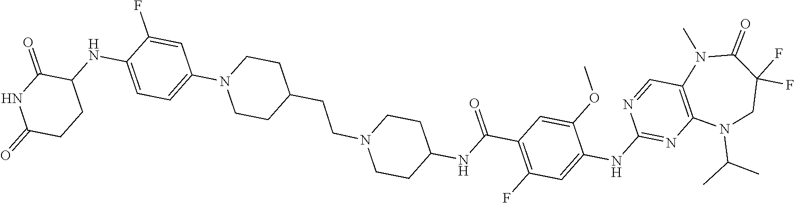 [SMILES: COc1cc(C(=O)NC2CCN(CCC3CCN(c4ccc(NC5CCC(=O)NC5=O)c(F)c4)CC3)CC2)c(F)cc1Nc1ncc2c(n1)N(C(C)C)CC(F)(F)C(=O)N2C] |
| 38 | 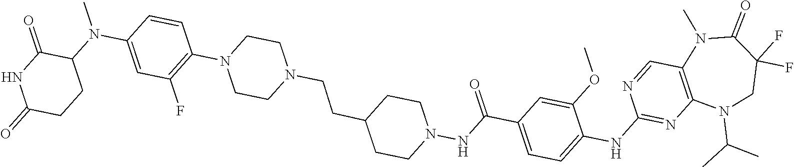 [SMILES: COc1cc(C(=O)NN2CCC(CCN3CCN(c4ccc(N(C)C5CCC(=O)NC5=O)cc4F)CC3)CC2)ccc1Nc1ncc2c(n1)N(C(C)C)CC(F)(F)C(=O)N2C] |
| 39 | 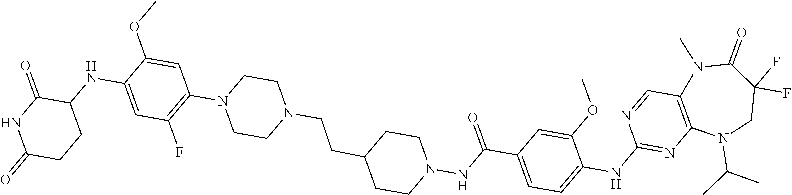 [SMILES: COc1cc(C(=O)NN2CCC(CCN3CCN(c4cc(OC)c(NC5CCC(=O)NC5=O)cc4F)CC3)CC2)ccc1Nc1ncc2c(n1)N(C(C)C)CC(F)(F)C(=O)N2C] |
| 40 | 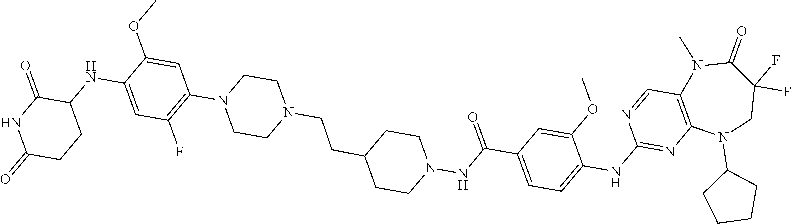 [SMILES: COc1cc(C(=O)NN2CCC(CCN3CCN(c4cc(OC)c(NC5CCC(=O)NC5=O)cc4F)CC3)CC2)ccc1Nc1ncc2c(n1)N(C1CCCC1)CC(F)(F)C(=O)N2C] |
| 41 | 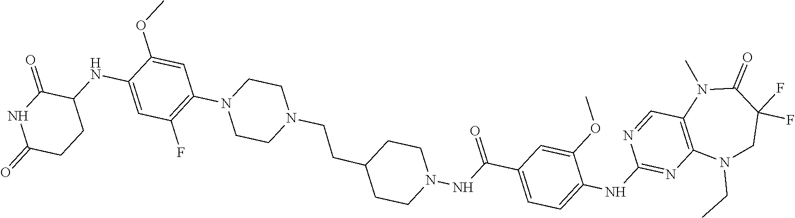 [SMILES: CCN1CC(F)(F)C(=O)N(C)c2cnc(Nc3ccc(C(=O)NN4CCC(CCN5CCN(c6cc(OC)c(NC7CCC(=O)NC7=O)cc6F)CC5)CC4)cc3OC)nc21] |
| 42 | 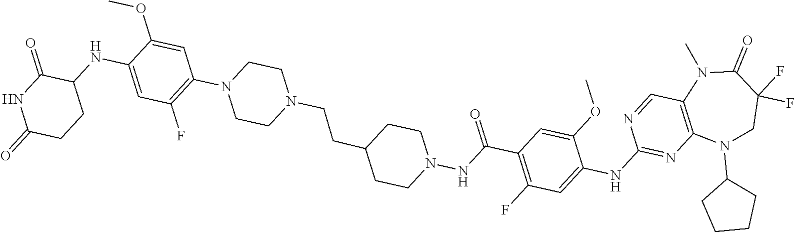 [SMILES: COc1cc(C(=O)NN2CCC(CCN3CCN(c4cc(OC)c(NC5CCC(=O)NC5=O)cc4F)CC3)CC2)c(F)cc1Nc1ncc2c(n1)N(C1CCCC1)CC(F)(F)C(=O)N2C] |
| 43 | 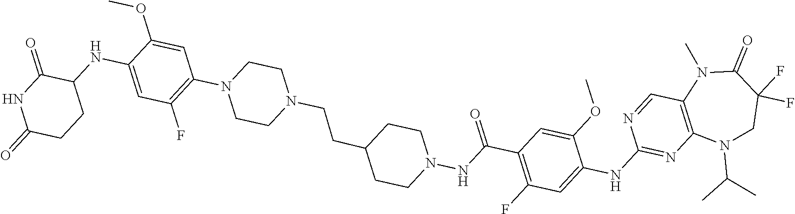 [SMILES: COc1cc(C(=O)NN2CCC(CCN3CCN(c4cc(OC)c(NC5CCC(=O)NC5=O)cc4F)CC3)CC2)c(F)cc1Nc1ncc2c(n1)N(C(C)C)CC(F)(F)C(=O)N2C] |
| 44 | 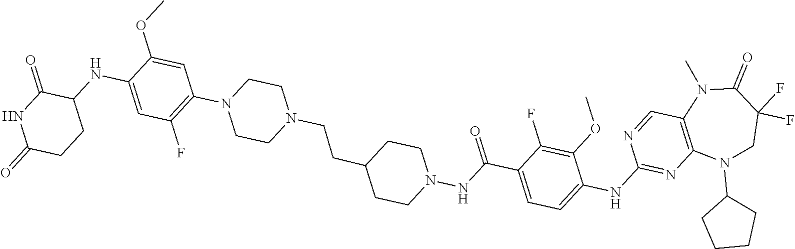 [SMILES: COc1cc(N2CCN(CCC3CCN(NC(=O)c4ccc(Nc5ncc6c(n5)N(C5CCCC5)CC(F)(F)C(=O)N6C)c(OC)c4F)CC3)CC2)c(F)cc1NC1CCC(=O)NC1=O] |
| 45 | 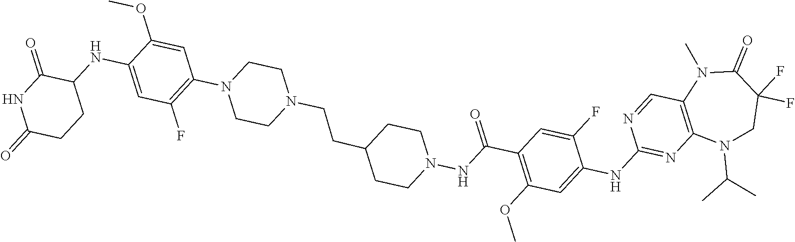 [SMILES: COc1cc(N2CCN(CCC3CCN(NC(=O)c4cc(F)c(Nc5ncc6c(n5)N(C(C)C)CC(F)(F)C(=O)N6C)cc4OC)CC3)CC2)c(F)cc1NC1CCC(=O)NC1=O] |
| 46 | 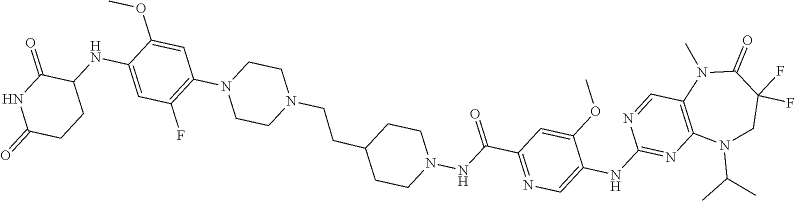 [SMILES: COc1cc(C(=O)NN2CCC(CCN3CCN(c4cc(OC)c(NC5CCC(=O)NC5=O)cc4F)CC3)CC2)ncc1Nc1ncc2c(n1)N(C(C)C)CC(F)(F)C(=O)N2C] |
| 47 | 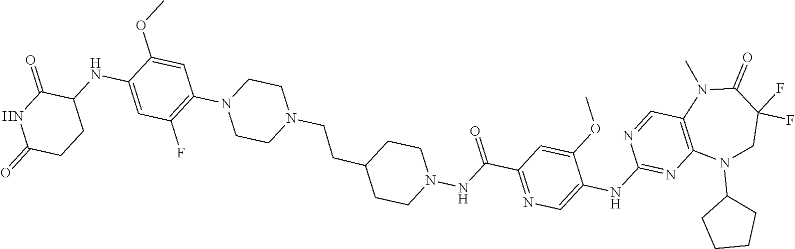 [SMILES: COc1cc(C(=O)NN2CCC(CCN3CCN(c4cc(OC)c(NC5CCC(=O)NC5=O)cc4F)CC3)CC2)ncc1Nc1ncc2c(n1)N(C1CCCC1)CC(F)(F)C(=O)N2C] |
| 48 | 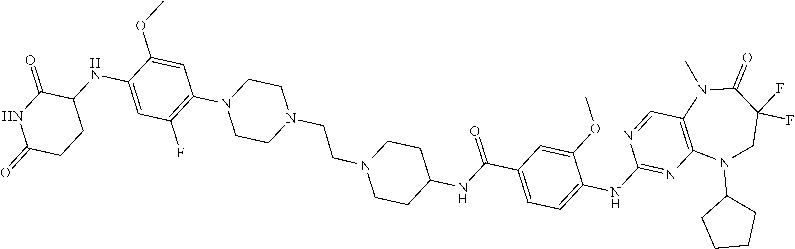 [SMILES: COc1cc(C(=O)NC2CCN(CCN3CCN(c4cc(OC)c(NC5CCC(=O)NC5=O)cc4F)CC3)CC2)ccc1Nc1ncc2c(n1)N(C1CCCC1)CC(F)(F)C(=O)N2C] |
| 49 | 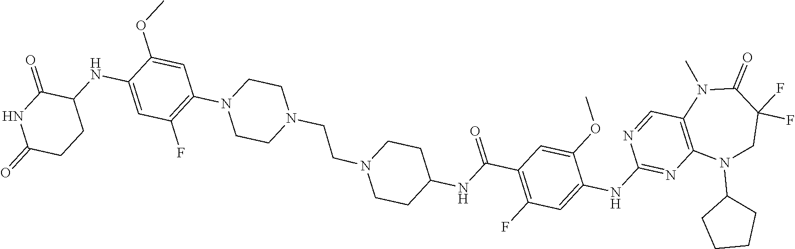 [SMILES: COc1cc(C(=O)NC2CCN(CCN3CCN(c4cc(OC)c(NC5CCC(=O)NC5=O)cc4F)CC3)CC2)c(F)cc1Nc1ncc2c(n1)N(C1CCCC1)CC(F)(F)C(=O)N2C] |
| 50 | 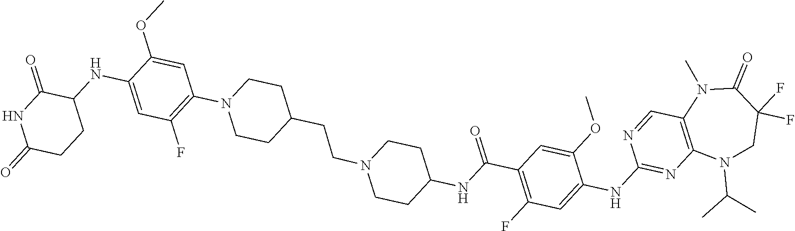 [SMILES: COc1cc(C(=O)NC2CCN(CCC3CCN(c4cc(OC)c(NC5CCC(=O)NC5=O)cc4F)CC3)CC2)c(F)cc1Nc1ncc2c(n1)N(C(C)C)CC(F)(F)C(=O)N2C] |
| 51 | 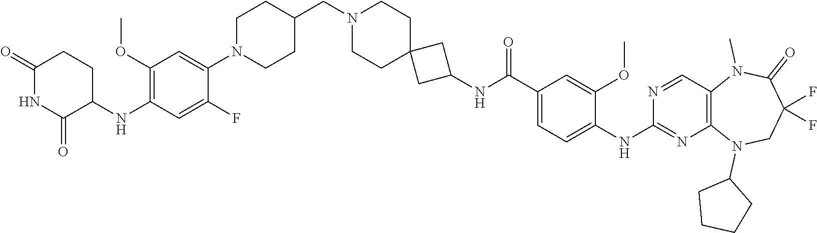 [SMILES: COc1cc(C(=O)NC2CC3(CCN(CC4CCN(c5cc(OC)c(NC6CCC(=O)NC6=O)cc5F)CC4)CC3)C2)ccc1Nc1ncc2c(n1)N(C1CCCC1)CC(F)(F)C(=O)N2C] |
| 52 | 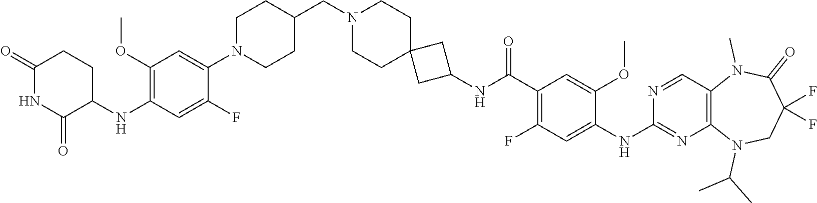 [SMILES: COc1cc(C(=O)NC2CC3(CCN(CC4CCN(c5cc(OC)c(NC6CCC(=O)NC6=O)cc5F)CC4)CC3)C2)c(F)cc1Nc1ncc2c(n1)N(C(C)C)CC(F)(F)C(=O)N2C] |
| 53 | 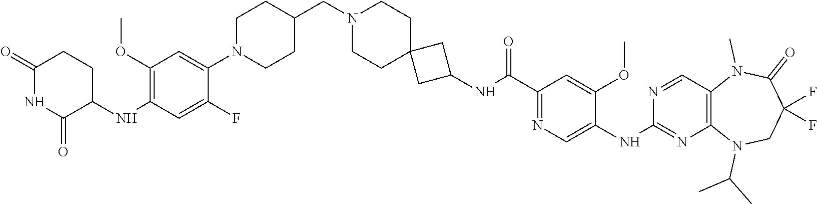 [SMILES: COc1cc(C(=O)NC2CC3(CCN(CC4CCN(c5cc(OC)c(NC6CCC(=O)NC6=O)cc5F)CC4)CC3)C2)ncc1Nc1ncc2c(n1)N(C(C)C)CC(F)(F)C(=O)N2C] |
| 54 | 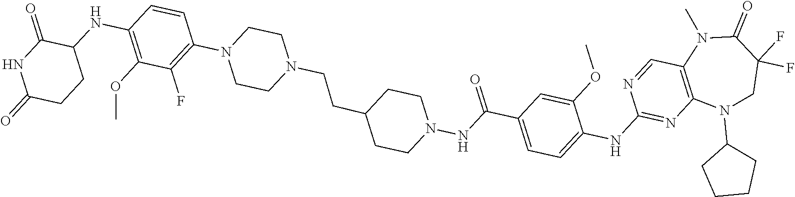 [SMILES: COc1cc(C(=O)NN2CCC(CCN3CCN(c4ccc(NC5CCC(=O)NC5=O)c(OC)c4F)CC3)CC2)ccc1Nc1ncc2c(n1)N(C1CCCC1)CC(F)(F)C(=O)N2C] |
| 55 | 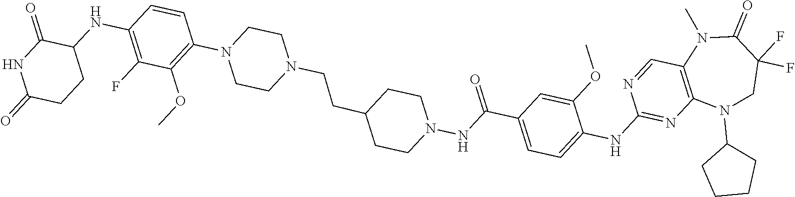 [SMILES: COc1cc(C(=O)NN2CCC(CCN3CCN(c4ccc(NC5CCC(=O)NC5=O)c(F)c4OC)CC3)CC2)ccc1Nc1ncc2c(n1)N(C1CCCC1)CC(F)(F)C(=O)N2C] |
| 56 | 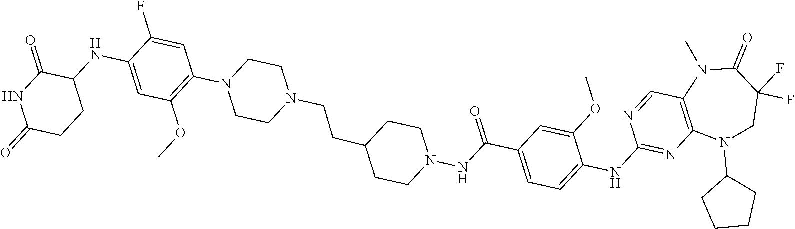 [SMILES: COc1cc(C(=O)NN2CCC(CCN3CCN(c4cc(F)c(NC5CCC(=O)NC5=O)cc4OC)CC3)CC2)ccc1Nc1ncc2c(n1)N(C1CCCC1)CC(F)(F)C(=O)N2C] |
| 57 | 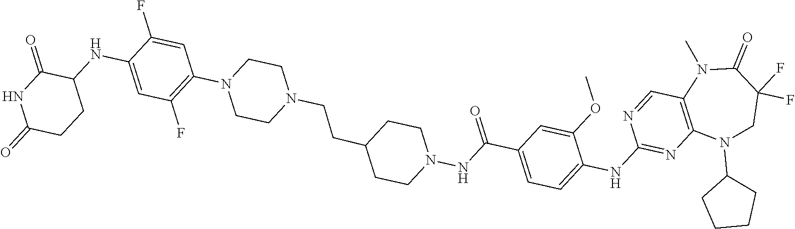 [SMILES: COc1cc(C(=O)NN2CCC(CCN3CCN(c4cc(F)c(NC5CCC(=O)NC5=O)cc4F)CC3)CC2)ccc1Nc1ncc2c(n1)N(C1CCCC1)CC(F)(F)C(=O)N2C] |
| 58 | 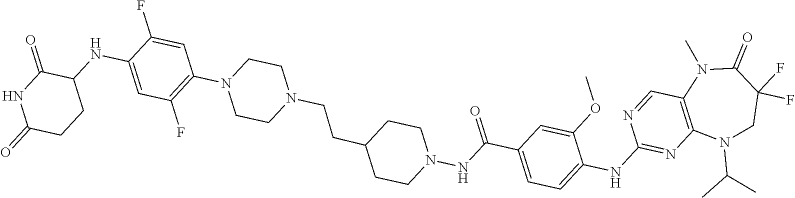 [SMILES: COc1cc(C(=O)NN2CCC(CCN3CCN(c4cc(F)c(NC5CCC(=O)NC5=O)cc4F)CC3)CC2)ccc1Nc1ncc2c(n1)N(C(C)C)CC(F)(F)C(=O)N2C] |
| 59 | 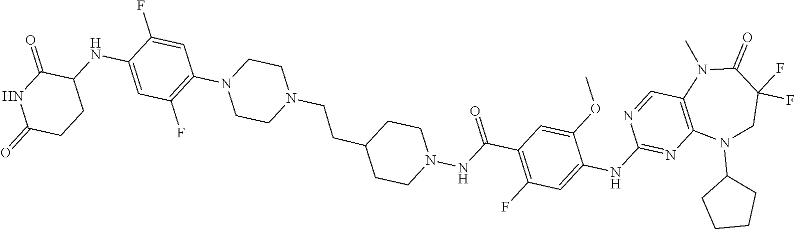 [SMILES: COc1cc(C(=O)NN2CCC(CCN3CCN(c4cc(F)c(NC5CCC(=O)NC5=O)cc4F)CC3)CC2)c(F)cc1Nc1ncc2c(n1)N(C1CCCC1)CC(F)(F)C(=O)N2C] |
| 60 | 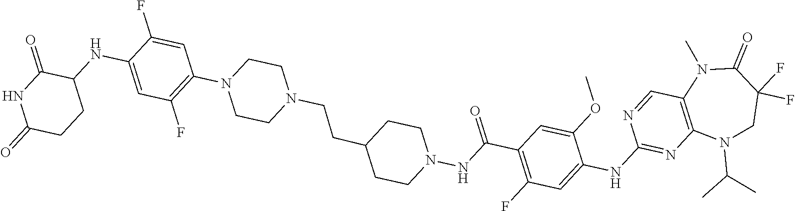 [SMILES: COc1cc(C(=O)NN2CCC(CCN3CCN(c4cc(F)c(NC5CCC(=O)NC5=O)cc4F)CC3)CC2)c(F)cc1Nc1ncc2c(n1)N(C(C)C)CC(F)(F)C(=O)N2C] |
| 61 | 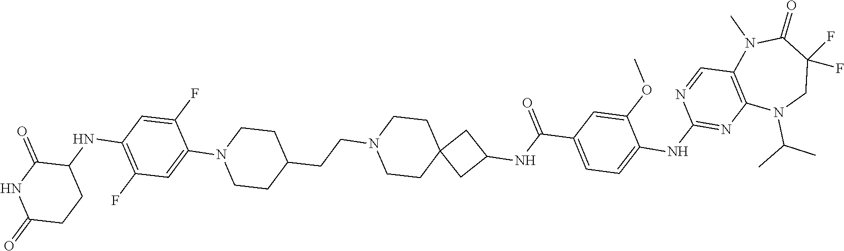 [SMILES: COc1cc(C(=O)NC2CC3(CCN(CCC4CCN(c5cc(F)c(NC6CCC(=O)NC6=O)cc5F)CC4)CC3)C2)ccc1Nc1ncc2c(n1)N(C(C)C)CC(F)(F)C(=O)N2C] |
| 62 | 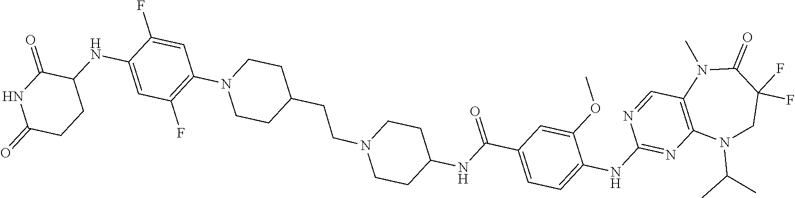 [SMILES: COc1cc(C(=O)NC2CCN(CCC3CCN(c4cc(F)c(NC5CCC(=O)NC5=O)cc4F)CC3)CC2)ccc1Nc1ncc2c(n1)N(C(C)C)CC(F)(F)C(=O)N2C] |
| 63 | 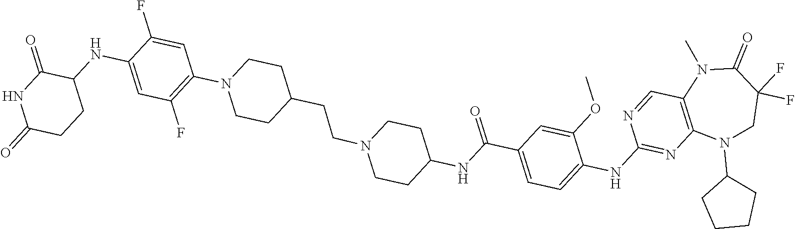 [SMILES: COc1cc(C(=O)NC2CCN(CCC3CCN(c4cc(F)c(NC5CCC(=O)NC5=O)cc4F)CC3)CC2)ccc1Nc1ncc2c(n1)N(C1CCCC1)CC(F)(F)C(=O)N2C] |
| 64 | 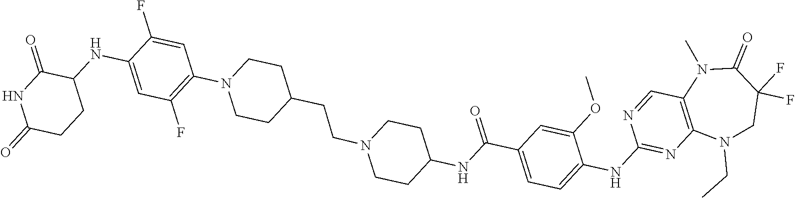 [SMILES: CCN1CC(F)(F)C(=O)N(C)c2cnc(Nc3ccc(C(=O)NC4CCN(CCC5CCN(c6cc(F)c(NC7CCC(=O)NC7=O)cc6F)CC5)CC4)cc3OC)nc21] |
| 65 | 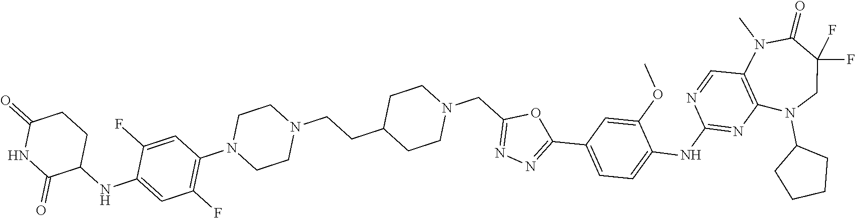 [SMILES: COc1cc(-c2nnc(CN3CCC(CCN4CCN(c5cc(F)c(NC6CCC(=O)NC6=O)cc5F)CC4)CC3)o2)ccc1Nc1ncc2c(n1)N(C1CCCC1)CC(F)(F)C(=O)N2C] |
| 66 | 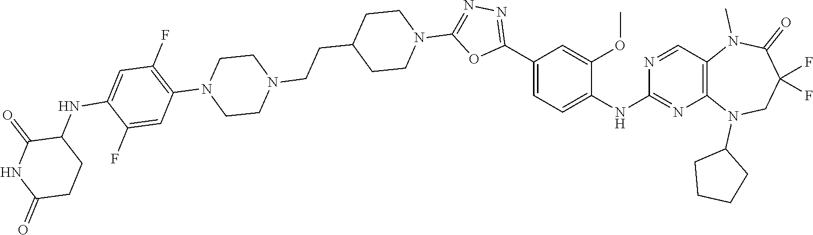 [SMILES: COc1cc(-c2nnc(N3CCC(CCN4CCN(c5cc(F)c(NC6CCC(=O)NC6=O)cc5F)CC4)CC3)o2)ccc1Nc1ncc2c(n1)N(C1CCCC1)CC(F)(F)C(=O)N2C] |
| 67 | 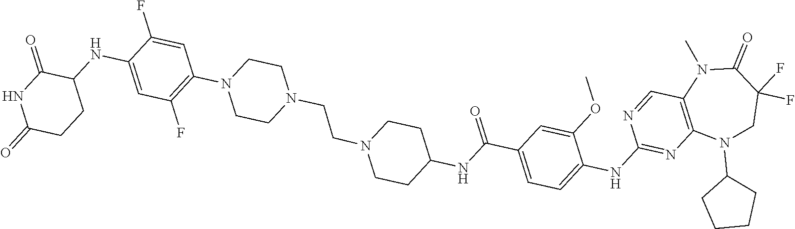 [SMILES: COc1cc(C(=O)NC2CCN(CCN3CCN(c4cc(F)c(NC5CCC(=O)NC5=O)cc4F)CC3)CC2)ccc1Nc1ncc2c(n1)N(C1CCCC1)CC(F)(F)C(=O)N2C] |
| 68 | 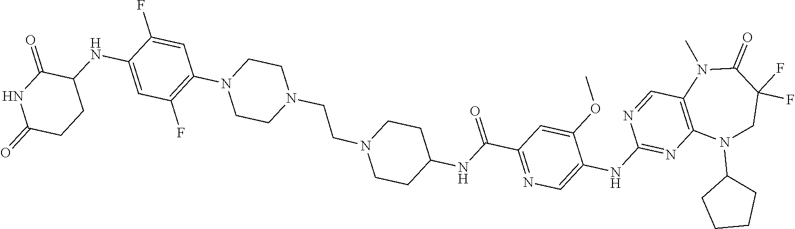 [SMILES: COc1cc(C(=O)NC2CCN(CCN3CCN(c4cc(F)c(NC5CCC(=O)NC5=O)cc4F)CC3)CC2)ncc1Nc1ncc2c(n1)N(C1CCCC1)CC(F)(F)C(=O)N2C] |
| 69 | 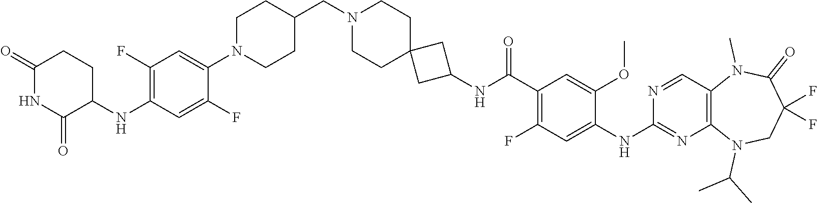 [SMILES: COc1cc(C(=O)NC2CC3(CCN(CC4CCN(c5cc(F)c(NC6CCC(=O)NC6=O)cc5F)CC4)CC3)C2)c(F)cc1Nc1ncc2c(n1)N(C(C)C)CC(F)(F)C(=O)N2C] |
| 70 | 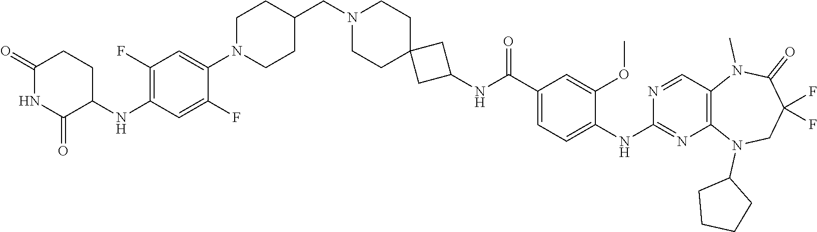 [SMILES: COc1cc(C(=O)NC2CC3(CCN(CC4CCN(c5cc(F)c(NC6CCC(=O)NC6=O)cc5F)CC4)CC3)C2)ccc1Nc1ncc2c(n1)N(C1CCCC1)CC(F)(F)C(=O)N2C] |
| 71 | 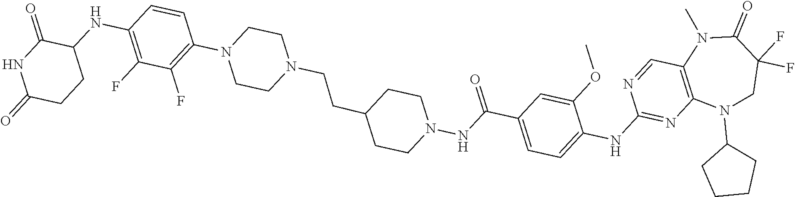 [SMILES: COc1cc(C(=O)NN2CCC(CCN3CCN(c4ccc(NC5CCC(=O)NC5=O)c(F)c4F)CC3)CC2)ccc1Nc1ncc2c(n1)N(C1CCCC1)CC(F)(F)C(=O)N2C] |
| 72 | 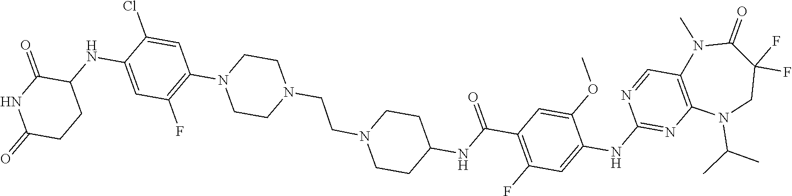 [SMILES: COc1cc(C(=O)NC2CCN(CCN3CCN(c4cc(Cl)c(NC5CCC(=O)NC5=O)cc4F)CC3)CC2)c(F)cc1Nc1ncc2c(n1)N(C(C)C)CC(F)(F)C(=O)N2C] |
| 73 | 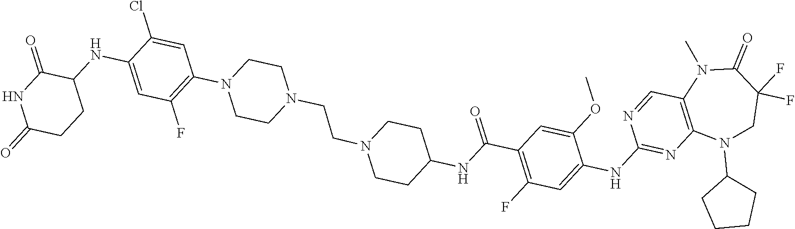 [SMILES: COc1cc(C(=O)NC2CCN(CCN3CCN(c4cc(Cl)c(NC5CCC(=O)NC5=O)cc4F)CC3)CC2)c(F)cc1Nc1ncc2c(n1)N(C1CCCC1)CC(F)(F)C(=O)N2C] |
| 74 | 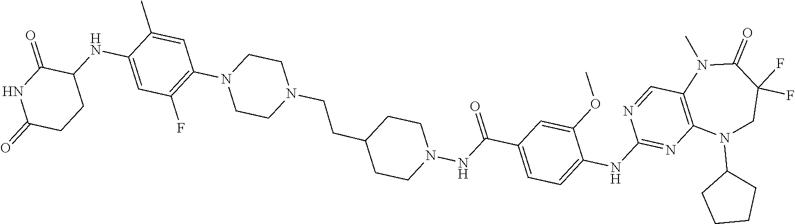 [SMILES: COc1cc(C(=O)NN2CCC(CCN3CCN(c4cc(C)c(NC5CCC(=O)NC5=O)cc4F)CC3)CC2)ccc1Nc1ncc2c(n1)N(C1CCCC1)CC(F)(F)C(=O)N2C] |
| 75 | 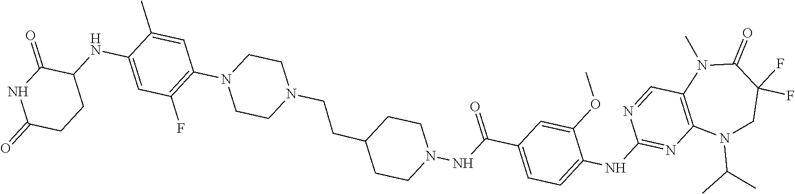 [SMILES: COc1cc(C(=O)NN2CCC(CCN3CCN(c4cc(C)c(NC5CCC(=O)NC5=O)cc4F)CC3)CC2)ccc1Nc1ncc2c(n1)N(C(C)C)CC(F)(F)C(=O)N2C] |
| 76 | 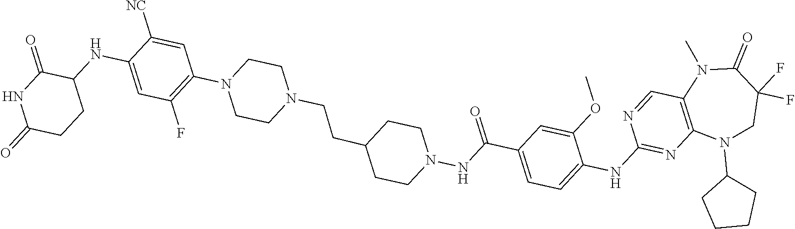 [SMILES: COc1cc(C(=O)NN2CCC(CCN3CCN(c4cc(C#N)c(NC5CCC(=O)NC5=O)cc4F)CC3)CC2)ccc1Nc1ncc2c(n1)N(C1CCCC1)CC(F)(F)C(=O)N2C] |
| 77 | 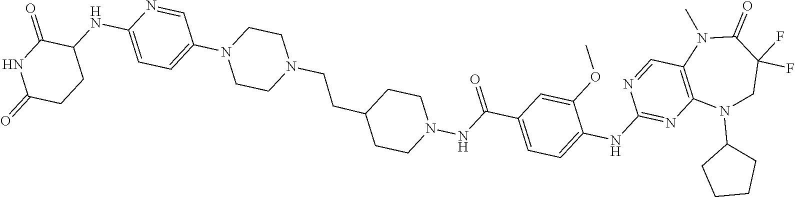 [SMILES: COc1cc(C(=O)NN2CCC(CCN3CCN(c4ccc(NC5CCC(=O)NC5=O)nc4)CC3)CC2)ccc1Nc1ncc2c(n1)N(C1CCCC1)CC(F)(F)C(=O)N2C] |

The compounds of the present invention were purified according to the following method and the structure was analyzed.

### Instruments

LCMS: Shimadzu LCMS-2020, Agilent 1200/G6110A, Agilent 1200/G1956A
HPLC: Agilent 1260 II LC, Agilent 1200/G6410B
NMR: BRUKER AVANCE III/400 MHz

### LCMS Analysis

LCMS data were recorded with Shimadzu LCMS-2020 or Agilent 1200/G6110A or Agilent 1200/G1956A equipped with an ESI (Electron Spray Ionization) device. 0.0375% TFA in water (solvent A) and 0.01875% TFA in ACN (solvent B) or 0.025% NH₃·H₂O in water (solvent A) and ACN (solvent B) were used as mobile phases. As a column, Kinetex EVO C18 (2.1×30 mm, 5 μm) or HALO C18 (3.0×30 mm, 2.7 μm) were used.

### HPLC Analysis

In HPLC analysis, Agilent 1260 II LC or Agilent 1200/G6410B were used. 0.0375% TFA in water (solvent A) and 0.01875% TFA in ACN (solvent B) were used as the mobile phase. As a column, Zobrax Eclipse Plus C18 (4.6×150 mm, 3.5 μm) or YMC ODS A (4.6×150 mm, 3 μm) were used.

### NMR Analysis

¹H NMR spectrum was recorded with Bruker AVANCE III 400 MHz/5 mm Probe (BBO).

### Example 1. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(3-(2,6-dioxopiperidin-3-yl)phenyl)-2,7-diazaspiro[3.5]nonan-2-yl)piperidin-1-yl)-3-methoxybenzamide (Compound 1)

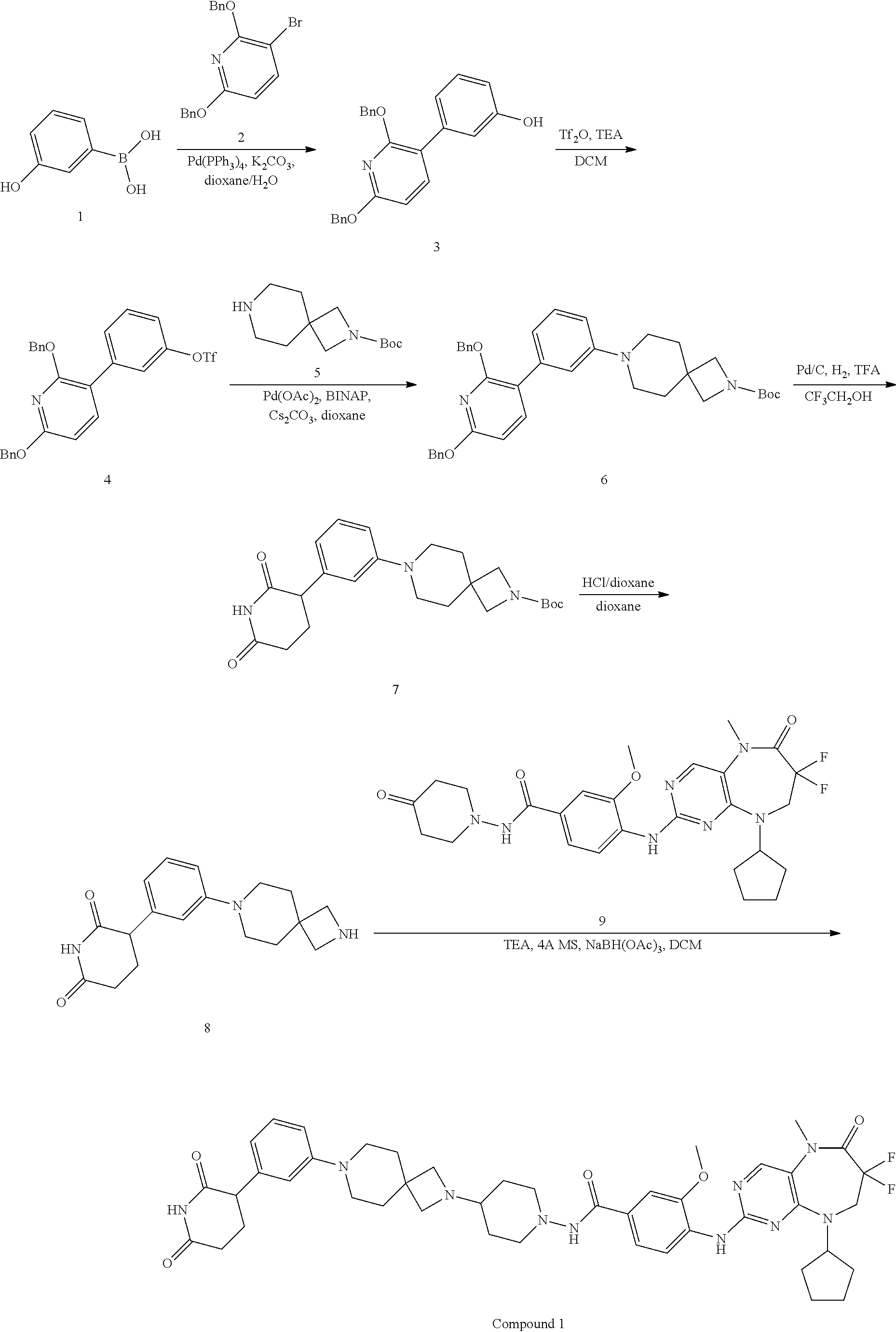

### Step 1. Synthesis of 3-(2,6-bis(benzyloxy)pyridin-3-yl)phenol (3)

To a solution of 2,6-bis(benzyloxy)-3-bromopyridine (1.9 g, 5.13 mmol) and 3-hydroxyphenylboronic acid (920.17 mg, 6.67 mmol) in dioxane (20 mL) and H₂O (5 mL) were added Pd(PPh₃)₄ (296.51 mg, 256.59 μmol) and K₂CO₃ (2.13 g, 15.40 mmol). The resulting mixture was stirred at 100° C. for 2 h under N₂. LCMS showed the starting material was consumed completely and a peak (59%) with desired mass. The reaction mixture was concentrated in vacuum. The residue was purified by flash silica gel chromatography (20 g SepaFlash® Silica Flash Column, Eluent of 0-12% EtOAc/Petroleum ether gradient @ 100 mL/min) to afford 3-(2,6-bis(benzyloxy)pyridin-3-yl)phenol (2 g, 5.11 mmol, 99.61% yield, 98% purity) as a yellow oil. MS(M+H)⁺=384.3.

### Step 2. Synthesis of 3-(2,6-bis(benzyloxy)pyridin-3-yl)phenyl trifluoromethanesulfonate (4)

To a solution of 3-(2,6-bis(benzyloxy)pyridin-3-yl)phenol (1 g, 2.61 mmol) in DCM (20 mL) were added TEA (1.06 g, 10.43 mmol, 1.45 mL) and Tf₂O (1.38 g, 4.88 mmol, 804.66 μL) at 0° C. The resulting mixture was stirred at 20° C. for 12 h. LCMS showed the starting material was consumed completely and a peak (87%) with desired mass. The reaction mixture was concentrated in vacuum. The residue was purified by flash silica gel chromatography (20 g SepaFlash® Silica Flash Column, Eluent of 0-1% EtOAc/Petroleum ether gradient @ 100 mL/min) to afford 3-(2,6-bis(benzyloxy)pyridin-3-yl)phenyl trifluoromethanesulfonate (448 mg, 842.99 mol, 32.32% yield, 97% purity) as a colorless oil. MS(M+H)⁺=516.1.

### Step 3. Synthesis of tert-butyl 7-(3-(2,6-bis(benzyloxy)pyridin-3-yl)phenyl)-2,7-diazaspiro[3.5]nonane-2-carboxylate (6)

To a solution of 3-(2,6-bis(benzyloxy)pyridin-3-yl)phenyl trifluoromethanesulfonate (395 mg, 766.25 μmol) and tert-butyl 2,7-diazaspiro[3.5]nonane-2-carboxylate (208.10 mg, 919.50 μmol) in dioxane (6 mL) were added BINAP (47.71 mg, 76.62 μmol), Pd(OAc)₂ (17.20 mg, 76.62 μmol) and Cs₂CO₃ (748.97 mg, 2.30 mmol). The resulting mixture was stirred at 100° C. for 12 h under N₂. LCMS showed the starting material was consumed completely and a peak (54%) with desired mass. The reaction mixture was concentrated in vacuum. The residue was purified by flash silica gel chromatography (10 g SepaFlash® Silica Flash Column, Eluent of 0-15% EtOAc/Petroleum ether gradient @ 100 mL/min) to afford tert-butyl 7-(3-(2,6-bis(benzyloxy)pyridin-3-yl)phenyl)-2,7-diazaspiro[3.5]nonane-2-carboxylate (396 mg, 669.22 μmol, 87.34% yield) as a yellow oil. MS(M+H)⁺=592.3.

### Step 4. Synthesis of tert-butyl 7-(3-(2,6-dioxopiperidin-3-yl)phenyl)-2,7-diazaspiro[3.5]nonane-2-carboxylate (7)

To a solution of tert-butyl 7-(3-(2,6-bis(benzyloxy)pyridin-3-yl)phenyl)-2,7-diazaspiro[3.5]nonane-2-carboxylate (396 mg, 669.22 μmol) in CF₃CH₂OH (10 mL) were added Pd/C (0.1 g, 10% purity) and TFA (76.31 mg, 669.22 μmol, 49.71 μL). The suspension was degassed and purged with H₂ for 3 times. The resulting mixture was stirred at 20° C. for 12 h under H₂ (15 Psi). LCMS showed the starting material was consumed completely and a main peak with desired mass. The reaction mixture was diluted with CF₃CH₂OH (30 mL) and filtered. The filtrate was adjusted to pH=7 using DIPEA. The resulting mixture was concentrated in vacuum. The residue was purified by flash silica gel chromatography (12 g SepaFlash® Silica Flash Column, Eluent of 0-80% EtOAc/Petroleum ether gradient @ 100 mL/min) to afford tert-butyl 7-(3-(2,6-dioxopiperidin-3-yl)phenyl)-2,7-diazaspiro[3.5]nonane-2-carboxylate (76 mg, 167.25 mol, 24.99% yield, 91% purity) as a yellow oil. MS(M+H)⁺=414.4.

### Step 5. Synthesis of 3-(3-(2,7-diazaspiro[3.5]nonan-7-yl)phenyl)piperidine-2,6-dione (8)

To a solution of tert-butyl 7-(3-(2,6-dioxopiperidin-3-yl)phenyl)-2,7-diazaspiro[3.5]nonane-2-carboxylate (76 mg, 183.79 mol) in dioxane (1 mL) was added HCl/dioxane (4 M, 2 mL). The resulting mixture was stirred at 20° C. for 1 h. LCMS showed the starting material was consumed completely and a main peak with desired mass. The reaction mixture was concentrated in vacuum to afford 3-(3-(2,7-diazaspiro[3.5]nonan-7-yl)phenyl)piperidine-2,6-dione (65 mg, crude, HCl salt) as a white solid. MS(M+H)⁺=314.3.

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(3-(2,6-dioxopiperidin-3-yl)phenyl)-2,7-diazaspiro[3.5]nonan-2-yl)piperidin-1-yl)-3-methoxybenzamide (Compound 1)

To a solution of 3-(3-(2,7-diazaspiro[3.5]nonan-7-yl)phenyl)piperidine-2,6-dione (61.79 mg, 176.61 μmol, HCl salt) in DCM (4 mL) were added TEA (44.68 mg, 441.53 μmol, 61.46 μL), 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(4-oxopiperidin-1-yl)benzamide (80 mg, 147.18 mol) and 4A MS (50 mg). The mixture was stirred at 20° C. for 0.5 h. NaBH(OAc)₃ (93.58 mg, 441.53 μmol) was slowly added to the reaction mixture. The resulting mixture was stirred at 20° C. for 12 h. LCMS showed the starting material was consumed completely and a peak (63%) with desired mass. The reaction mixture was filtered. The filtrate was concentrated in vacuum. The residue was purified by prep-TLC (SiO₂, DCM:MeOH=10:1) to afford a crude. The crude was dissolved in mix solution (20 mL, ACN:H₂O=1:3). The eluent was lyophilized to afford a residue (51 mg, 93% purity). The residue was re-purified by prep-HPLC (column: Waters Xbridge 150*25 mm*5 μm; mobile phase: [water (NH₄HCO₃)-ACN]; B %: 42%-72%, 8 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(3-(2,6-dioxopiperidin-3-yl)phenyl)-2,7-diazaspiro[3.5]nonan-2-yl)piperidin-1-yl)-3-methoxybenzamide (15.3 mg, 17.28 μmol, 11.74% yield, 95% purity) as a white solid. MS(M+H)⁺=841.4.

¹H NMR (400 MHz, DMSO-d₆) δ=10.79 (s, 1H), 9.32 (s, 1H), 8.31-8.20 (m, 2H), 7.95 (s, 1H), 7.49-7.37 (m, 2H), 7.13 (t, J=7.8 Hz, 1H), 6.86-6.76 (m, 2H), 6.58 (d, J=7.6 Hz, 1H), 4.83-4.70 (m, 1H), 4.11-3.98 (m, 2H), 3.92 (s, 3H), 3.75 (dd, J=4.8, 11.2 Hz, 1H), 3.33 (s, 3H), 3.12-3.03 (m, 4H), 3.02-2.88 (m, 6H), 2.79 (br t, J=8.9 Hz, 2H), 2.72-2.59 (m, 1H), 2.48-2.42 (m, 1H), 2.25-2.15 (m, 1H), 2.15-2.06 (m, 1H), 2.04-2.01 (m, 1H), 1.98-1.88 (m, 2H), 1.85-1.74 (m, 4H), 1.73-1.64 (m, 4H), 1.60-1.56 (m, 4H), 1.37-1.22 (m, 2H).

### Example 2. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(1-(4-(2,6-dioxopiperidin-3-yl)-2-fluorophenyl)pyrrolidin-3-yl)piperidin-1-yl)-3-methoxybenzamide (Compound 2)

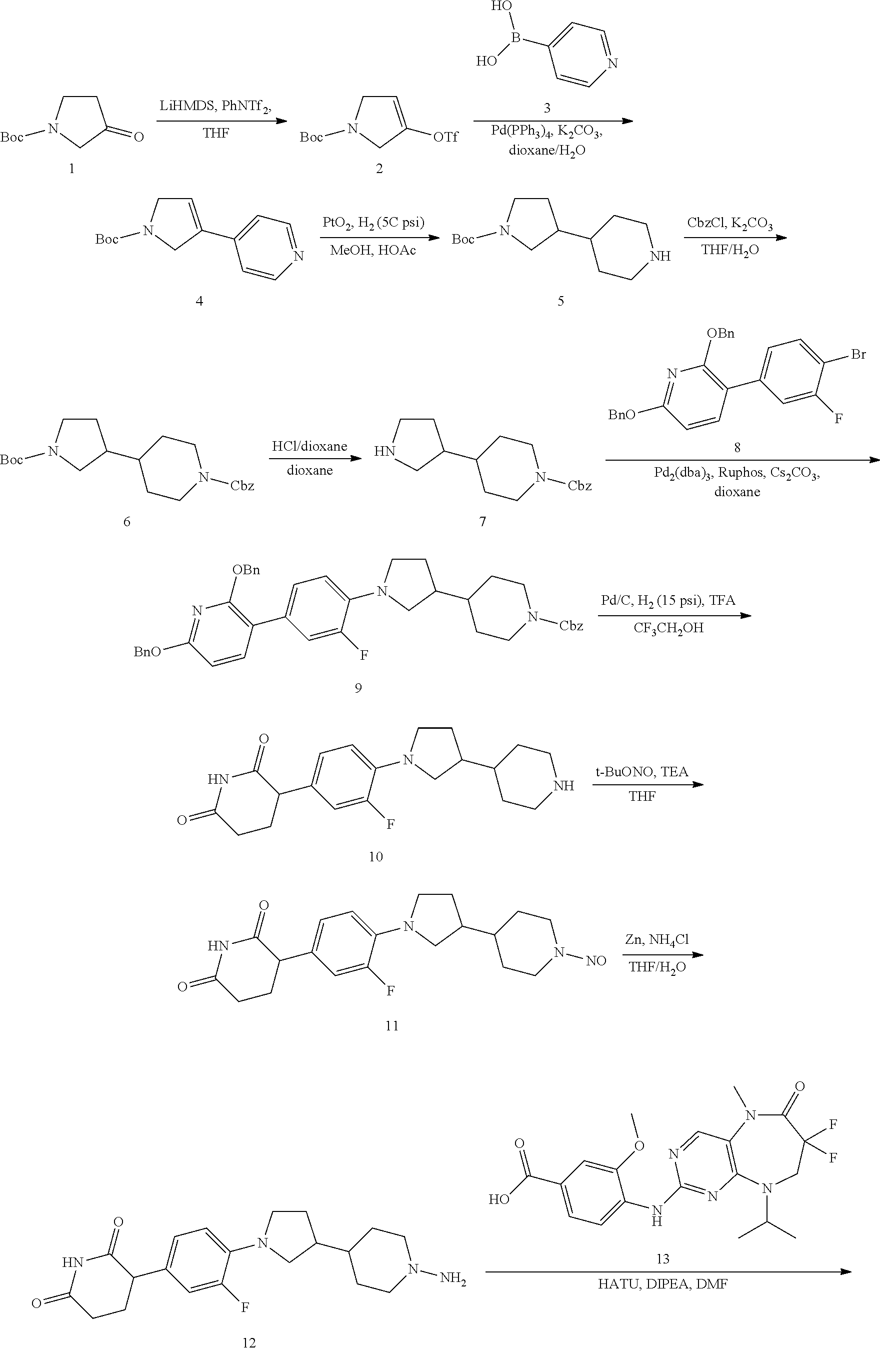

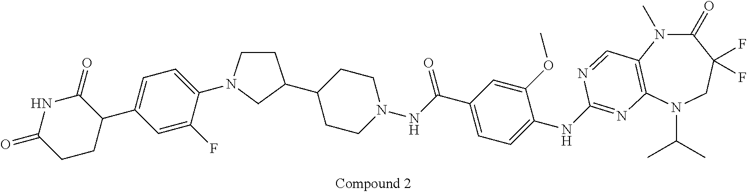 [SMILES: COc1cc(C(=O)NN2CCC(C3CCN(c4ccc(C5CCC(=O)NC5=O)cc4F)C3)CC2)ccc1Nc1ncc2c(n1)N(C(C)C)CC(F)(F)C(=O)N2C]

### Step 1. Synthesis of tert-butyl 3-(((trifluoromethyl)sulfonyl)oxy)-2,5-dihydro-1H-pyrrole-1-carboxylate (2)

To a solution of tert-butyl 3-oxopyrrolidine-1-carboxylate (15 g, 80.98 mmol) in THF (50 mL) was added LiHMDS (1 M, 97.18 mL) at −70° C. Then a solution of PhNTf₂ (31.83 g, 89.08 mmol) in THF (50 mL) was added dropwise after 0.5 h at −70° C. The resulting mixture was stirred at −70° C. for 2 h. TLC (petroleum ether:EtOAc=5:1) indicated several new spots formed. The reaction mixture was quenched with saturated NH₄Cl solution (200 mL) and extracted with EtOAc (100 mL×3). The combined organic layers were washed with saturated brine (150 mL), dried over Na₂SO₄, filtered. The filtrate was concentrated under reduced pressure to give tert-butyl 3-(((trifluoromethyl)sulfonyl)oxy)-2,5-dihydro-1H-pyrrole-1-carboxylate (30 g, crude) as a yellow oil. MS(M-t-Bu+H)⁺=261.7.

### Step 2. Synthesis of tert-butyl 3-(pyridin-4-yl)-2,5-dihydro-1H-pyrrole-1-carboxylate (4)

It was synthesized by similar method described in step 1 of Example 1 to obtain tert-butyl 3-(pyridin-4-yl)-2,5-dihydro-1H-pyrrole-1-carboxylate (4 g, 4.38 mmol, 9.27% yield, 27% purity) as a yellow oil. MS(M+H)⁺=247.1.

### Step 3. Synthesis of tert-butyl 3-(piperidin-4-yl)pyrrolidine-1-carboxylate (5)

To a solution of tert-butyl 3-(pyridin-4-yl)-2,5-dihydro-1H-pyrrole-1-carboxylate (3.8 g, 15.43 mmol) in MeOH (30 mL) was added PtO₂ (300 mg, 1.32 mmol) under N₂. The mixture was stirred at 50° C. for 12 h under H₂ (50 psi). LCMS showed tert-butyl 3-(pyridin-4-yl)-2,5-dihydro-1H-pyrrole-1-carboxylate was consumed completely and ~30% of intermediate state (m/z=249.2) was detected. AcOH (3.15 g, 52.45 mmol, 3 mL) was added to the mixture. The mixture was stirred at 50° C. for 12 h under H₂ (50 psi). LCMS showed a main peak with desired mass. The mixture was filtered to remove the catalyst. The filtrate was concentrated under reduced pressure to give a residue. The residue was dissolved into water (50 mL). The mixture was adjusted to pH=8 using saturated NaHCO₃ solution. The mixture was extracted with EtOAc (50 mL×8). The combined organic layers were washed with saturated brine (50 mL), dried over Na₂SO₄, filtered. The filtrate was concentrated under reduced pressure to afford tert-butyl 3-(piperidin-4-yl)pyrrolidine-1-carboxylate (4 g, crude) as a yellow oil. MS(M+H)⁺=255.1.

### Step 4. Synthesis of benzyl 4-(1-(tert-butoxycarbonyl)pyrrolidin-3-yl)piperidine-1-carboxylate (6)

To a solution of tert-butyl 3-(piperidin-4-yl)pyrrolidine-1-carboxylate (3.5 g, 13.76 mmol) in THF (10 mL) and H₂O (10 mL) were added K₂CO₃ (3.80 g, 27.52 mmol) and CbzCl (2.35 g, 13.76 mmol, 1.96 mL). The mixture was stirred at 25° C. for 2 h. LCMS showed a main peak with desired mass. The reaction mixture was diluted with water (80 mL) and extracted with EtOAc (50 mL×3). The combined organic layers were washed with saturated brine (100 mL), dried over Na₂SO₄, filtered. The filtrate and concentrated under reduced pressure to give a residue. The residue was purified by flash silica gel chromatography (ISCO®; 40 g SepaFlash® Silica Flash Column, Eluent of 0-20% petroleum ether:EtOAc gradient @ 80 mL/min) to give benzyl 4-(1-(tert-butoxycarbonyl)pyrrolidin-3-yl)piperidine-1-carboxylate (2.8 g, 7.21 mmol, 52.38% yield) as a yellow oil. MS(M−Boc+H)⁺=289.2.

### Step 5. Synthesis of benzyl 4-(pyrrolidin-3-yl)piperidine-1-carboxylate (7)

It was synthesized by similar method described in step 5 of Example i to obtain benzyl 4-(pyrrolidin-3-yl)piperidine-1-carboxylate (1.3 g, 3.64 mmol, 50.53% yield, 91% purity, HCl salt) as a gray solid. MS(M+H)⁺=288.9.

### Step 6. Synthesis of benzyl 4-(1-(4-(2,6-bis(benzyloxy)pyridin-3-yl)-2-fluorophenyl)pyrrolidin-3-yl)piperidine-1-carboxylate (9)

It was synthesized by similar method described in step 3 of Example 1, changed Pd(OAc)2 and BINAP to Pd₂(dba)₃ (56.38 mg, 61.57 μmol) and RuPhos (28.73 mg, 61.57 mol) to obtain benzyl 4-(1-(4-(2,6-bis(benzyloxy)pyridin-3-yl)-2-fluorophenyl)pyrrolidin-3-yl)piperidine-1-carboxylate (0.6 g, 844.00 mol, 27.42% yield, 94.5% purity) as a yellow oil. MS(M+H)⁺=672.5.

### Step 7. Synthesis of 3-(3-fluoro-4-(3-(piperidin-4-yl)pyrrolidin-1-yl)phenyl)piperidine-2,6-dione (10)

It was synthesized by similar method described in step 4 of Example i to obtain 3-(3-fluoro-4-(3-(piperidin-4-yl)pyrrolidin-1-yl)phenyl)piperidine-2,6-dione (420 mg, crude) as a yellow oil. MS(M+H)⁺=360.2.

### Step 8. Synthesis of 3-(3-fluoro-4-(3-(1-nitrosopiperidin-4-yl)pyrrolidin-1-yl)phenyl)piperidine-2,6-dione (11)

To a solution of 3-(3-fluoro-4-(3-(piperidin-4-yl)pyrrolidin-1-yl)phenyl)piperidine-2,6-dione (320 mg, 890.28 μmol) in THF (15 mL) were added t-BuONO (183.61 mg, 1.78 mmol, 211.78 μL) and TEA (315.30 mg, 3.12 mmol, 433.71 μL). The mixture was stirred at 60° C. for 12 h. LCMS showed ~54% of the desired mass. The reaction mixture was diluted with water (80 mL) and extracted with EtOAc (50 mL×3). The combined organic layers were washed with saturated brine (50 mL), dried over Na₂SO₄, filtered. The filtrate was concentrated under reduced pressure to give a residue. The residue was purified by flash silica gel chromatography (ISCO®; 20 g SepaFlash® Silica Flash Column, Eluent of 0-70% petroleum ether:EtOAc gradient @ 80 mL/min) to afford 3-(3-fluoro-4-(3-(1-nitrosopiperidin-4-yl)pyrrolidin-1-yl)phenyl)piperidine-2,6-dione (130 mg, 314.93 mol, 35.37% yield, 94.1% purity) as a yellow solid. MS(M+H)⁺=388.9.

### Step 9. Synthesis of 3-(4-(3-(1-aminopiperidin-4-yl)pyrrolidin-1-yl)-3-fluorophenyl)piperidine-2,6-dione (12)

To a solution of 3-(3-fluoro-4-(3-(1-nitrosopiperidin-4-yl)pyrrolidin-1-yl)phenyl)piperidine-2,6-dione (130 mg, 334.68 mol) in THF (8 mL) were added Zn (109.42 mg, 1.67 mmol) and a solution of NH₄Cl (53.71 mg, 1.00 mmol) in H₂O (2 mL) at 0° C. The mixture was stirred at 25° C. for 12 h. LCMS showed ~85% of desired mass. The mixture was filtered to remove the solid. The filter cake was washed with THF (50 mL). The filtrated was concentrated under reduced pressure. The residue was purified by prep-HPLC (column: Phenomenex luna C18 150×25 mm×10 μm; mobile phase: [water (FA)-ACN]; B %: 0%-25%, 10 min). The eluent was lyophilized to afford 3-(4-(3-(1-aminopiperidin-4-yl)pyrrolidin-1-yl)-3-fluorophenyl)piperidine-2,6-dione (70 mg, 186.94 mol, 55.86% yield, 100% purity) as a white solid. MS(M+H)⁺=375.0.

### Step 10. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(1-(4-(2,6-dioxopiperidin-3-yl)-2-fluorophenyl)pyrrolidin-3-yl)piperidin-1-yl)-3-methoxybenzamide (Compound 2)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (78.78 mg, 186.94 mol) in DMF (5 mL) were added DIPEA (72.48 mg, 560.82 μmol, 97.68 μL) and HATU (85.30 mg, 224.33 μmol). Then 3-(4-(3-(1-aminopiperidin-4-yl)pyrrolidin-1-yl)-3-fluorophenyl)piperidine-2,6-dione (70 mg, 186.94 mol) was added to the mixture after 0.5 h. The resulting mixture was stirred at 25° C. for 1 h. LCMS showed ~32% of desired mass. The reaction mixture was diluted with water (30 mL) and extracted with EtOAc (50 mL×3). The combined organic layers were washed with saturated brine (50 mL), dried over Na₂SO₄, filtered. The filtrate was concentrated under reduced pressure to give a residue. The residue was purified by flash silica gel chromatography (ISCO®; 20 g SepaFlash® Silica Flash Column, Eluent of 0-100% petroleum ether:EtOAc gradient @ 80 mL/min) to give a crude. The crude was purified by prep-HPLC (column: Waters xbridge 150×25 mm 10 μm; mobile phase: [water (NH₄HCO₃)-ACN]; B %: 32%-62%, 11 min) and repurified by prep-TLC (DCM:MeOH=10:1). The eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(4-(1-(4-(2,6-dioxopiperidin-3-yl)-2-fluorophenyl)pyrrolidin-3-yl)piperidin-1-yl)-3-methoxybenzamide (15.7 mg, 19.35 mol, 10.35% yield, 99.0% purity) as a white solid. MS(M+H)⁺=778.3.

¹H NMR (400 MHz, DMSO-d₆) δ=10.83-10.69 (m, 1H), 9.27 (s, 1H), 8.30 (d, J=8.1 Hz, 1H), 8.22 (s, 1H), 7.87 (s, 1H), 7.49-7.38 (m, 2H), 6.93 (d, J=15.4 Hz, 1H), 6.85 (d, J=8.4 Hz, 1H), 6.68 (t, J=8.9 Hz, 1H), 4.90-4.87 (m, 1H), 4.03 (t, J=13.6 Hz, 2H), 3.93 (s, 3H), 3.73 (dd, J=4.9, 11.6 Hz, 1H), 3.51-3.35 (m, 6H), 3.11-2.99 (m, 3H), 2.75 (t, J=11.1 Hz, 2H), 2.68-2.59 (m, 1H), 2.48-2.41 (m, 1H), 2.22-2.11 (m, 1H), 2.10-1.92 (m, 3H), 1.84-1.67 (m, 2H), 1.65-1.51 (m, 1H), 1.50-1.27 (m, 3H), 1.25-1.23 (m, J=6.6 Hz, 6H).

### Example 3. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(1-(4-(2,6-dioxopiperidin-3-yl)-2-fluorophenyl)azetidin-3-yl)piperidin-4-yl)-3-methoxybenzamide (Compound 3)

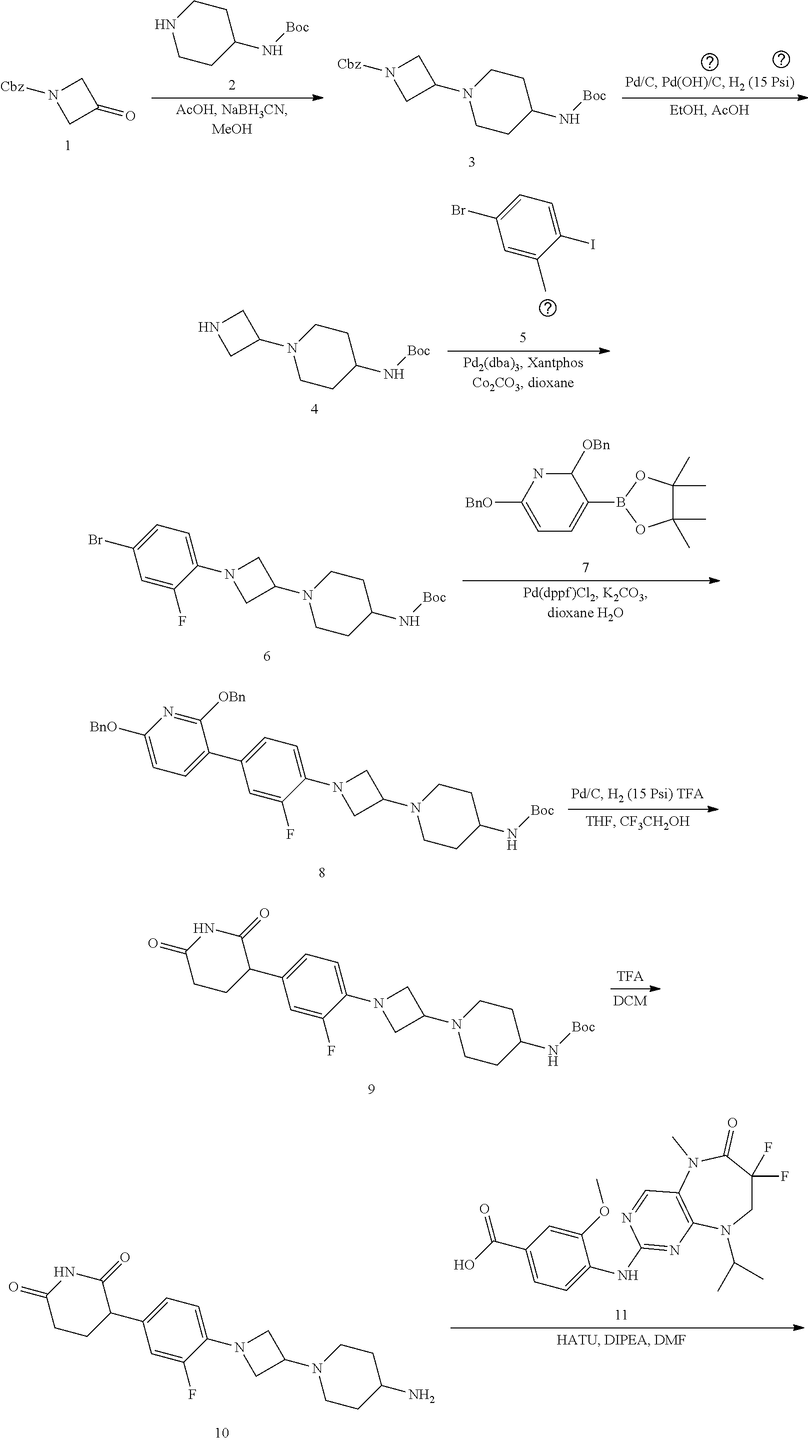

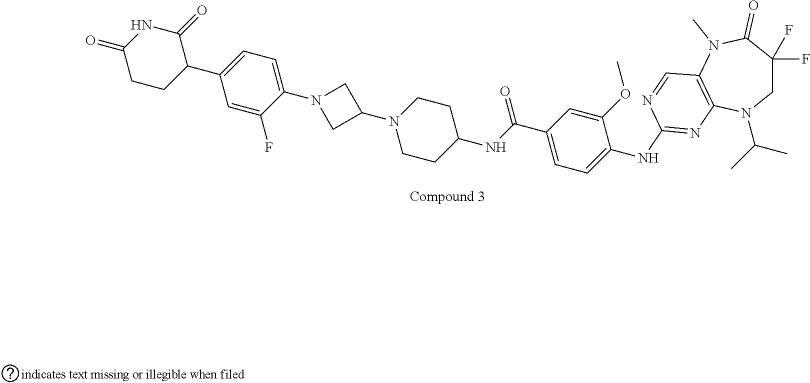 [SMILES: COc1cc(C(=O)NC2CCN(C3CN(c4ccc(C5CCC(=O)NC5=O)cc4F)C3)CC2)ccc1Nc1ncc2c(n1)N(C(C)C)CC(F)(F)C(=O)N2C]

### Step 1. Synthesis of benzyl 3-(4-((tert-butoxycarbonyl)amino)piperidin-1-yl)azetidine-1-carboxylate (3)

To a solution of benzyl 3-oxoazetidine-1-carboxylate (10 g, 48.73 mmol) and tert-butyl piperidin-4-ylcarbamate (9.76 g, 48.73 mmol) in MeOH (100 mL) was added AcOH (292.64 mg, 4.87 mmol, 278.97 μL). The mixture was stirred at 20° C. for 0.5 hr. Then NaBH₃CN (4.59 g, 73.10 mmol) was added and the resulting mixture was stirred at 20° C. for 16 hr. LCMS showed desired mass was detected. The mixture was concentrated under reduced pressure, the crude was diluted with water (100 mL) and extracted with EtOAc (100 mL×3). The combined organic layer was washed with NaHCO₃ (20 mL×2), dried over Na₂SO₄ and filtered. The filtrate was concentrated in vacuum. The residue was purified by flash silica gel chromatography (ISCO®; 120 g SepaFlash® Silica Flash Column, Eluent of 0~60% EtOAc/Petroleum ether gradient @100 mL/min) to afford 2 batches, batch 1: benzyl 3-(4-((tert-butoxycarbonyl)amino)piperidin-1-yl)azetidine-1-carboxylate (3.64 g, 9.35 mmol, 19.18% yield) as a white solid and batch 2: impure product, which was repurified by MPLC (0.1% FA condition; 330 g Flash Column; TELEDYNE ISCO CombiFlashRf₁₅₀ Welch Ultimate XB_C₁₉ 20-40 μm; 120 A; Solvent for sample dissolution about 4 grams of sample dissolved in 10 mL of DMF; Eluent of 5-50% ACN/H₂O gradient @ 100 mL/min, 35 min, 50% ACN/H₂O gradient @ 100 mL/min, 10 min), the eluent was freeze-dried to afford benzyl 3-(4-((tert-butoxycarbonyl)amino)piperidin-1-yl)azetidine-1-carboxylate (2.4 g, 6.16 mmol, 12.64% yield) as a white solid. MS(M+H)⁺=390.3.

### Step 2. Synthesis of tert-butyl (1-(azetidin-3-yl)piperidin-4-yl)carbamate (4)

To a solution of benzyl 3-(4-((tert-butoxycarbonyl)amino)piperidin-1-yl)azetidine-1-carboxylate (6.44 g, 16.53 mmol), AcOH (496.47 mg, 8.27 mmol, 473.28 μL) in EtOH (70 mL) were added Pd/C (600 mg, 10% purity) and Pd(OH)₂/C (600 mg, 20% purity) under N₂. The suspension was degassed and purged with H₂ for 3 times and stirred at 20° C. for 16 hr under H₂ (15 Psi). LCMS showed the starting material was consumed completely. The mixture was filtered. The filtrate was concentrated in vacuum to afford tert-butyl (1-(azetidin-3-yl)piperidin-4-yl)carbamate (5.6 g, crude) as a white solid, which was used directly. MS(M+H)⁺=256.2.

### Step 3. Synthesis of tert-butyl (1-(1-(4-bromo-2-fluorophenyl)azetidin-3-yl)piperidin-4-yl)carbamate (6)

It was synthesized by similar method described in step 6 of Example 2, changed RuPhos to Xantphos (91.73 mg, 158.53 μmol) to obtain tert-butyl (1-(1-(4-bromo-2-fluorophenyl)azetidin-3-yl)piperidin-4-yl)carbamate (540 mg, 1.26 mmol, 79.53% yield) as a yellow solid. MS(M+H)⁺=428.1.

### Step 4. Synthesis of tert-butyl (1-(1-(4-(2,6-bis(benzyloxy)pyridin-3-yl)-2-fluorophenyl)azetidin-3-yl)piperidin-4-yl)carbamate (8)

It was synthesized by similar method described in step 1 of Example 1, changed Pd(PPh₃)₄ to Pd(dppf)Cl₂ (68.33 mg, 93.38 μmol) to obtain tert-butyl (1-(1-(4-(2,6-bis(benzyloxy)pyridin-3-yl)-2-fluorophenyl)azetidin-3-yl)piperidin-4-yl) carbamate (586 mg, 917.39 μmol, 98.24% yield) as a yellow solid. MS(M+H)⁺=639.4.

### Step 5. Synthesis of tert-butyl (1-(1-(4-(2,6-dioxopiperidin-3-yl)-2-fluorophenyl)azetidin-3-yl)piperidin-4-yl)carb amate (9)

It was synthesized by similar method described in step 4 of Example 1, addition THF to obtain tert-butyl (1-(1-(4-(2,6-dioxopiperidin-3-yl)-2-fluorophenyl)azetidin-3-yl)piperidin-4-yl)carbamate (680 mg, crude) as a brown oil, which was used directly. MS(M+H)⁺=461.2.

### Step 6. Synthesis of 3-(4-(3-(4-aminopiperidin-1-yl)azetidin-1-yl)-3-fluorophenyl)piperidine-2,6-dione (10)

A mixture of tert-butyl (1-(1-(4-(2,6-dioxopiperidin-3-yl)-2-fluorophenyl)azetidin-3-yl)piperidin-4-yl)carbamate (340 mg, 738.26 μmol) and TFA (1 mL) in DCM (3 mL) was stirred at 20° C. for 1 hour. LCMS showed the starting material was consumed completely and 95% of desired mass was detected. The mixture was concentrated in vacuum to afford 3-(4-(3-(4-aminopiperidin-1-yl)azetidin-1-yl)-3-fluorophenyl)piperidine-2,6-dione (360 mg, TFA salt) as a brown gum, which was used directly. MS(M+H)⁺=361.2.

### Step 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(1-(4-(2,6-dioxopiperidin-3-yl)-2-fluorophenyl)azetidin-3-yl)piperidin-4-yl)-3-methoxybenzamide (Compound 3)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(1-(1-(4-(2,6-dioxopiperidin-3-yl)-2-fluorophenyl)azetidin-3-yl)piperidin-4-yl)-3-methoxybenzamide (211.5 mg, 204.71 mol, 47.92% yield, 96% purity, 2TFA salt) as a white solid. MS(M+H)⁺=764.3.

¹H NMR (400 MHz, CD₃OD) δ=8.16 (d, J=8.3 Hz, 1H), 8.13 (s, 1H), 7.65-7.49 (m, 2H), 6.98 (s, 1H), 6.97-6.93 (m, 1H), 6.63 (t, J=8.7 Hz, 1H), 5.11 (td, J=6.8, 13.5 Hz, 1H), 4.39-4.29 (m, 2H), 4.21 (br d, J=15.4 Hz, 2H), 4.17-4.07 (m, 4H), 4.01 (s, 3H), 3.80 (dd, J=5.5, 10.6 Hz, 1H), 3.76-3.58 (m, 2H), 3.39 (s, 3H), 3.27-3.03 (m, 2H), 2.81-2.57 (m, 2H), 2.42-2.25 (m, 2H), 2.25-2.13 (m, 2H), 2.11-1.83 (m, 2H), 1.32 (d, J=6.7 Hz, 6H).

### Example 4. Synthesis of N(7-((1-(4-(2,6-dioxopiperidin-3-yl)-2-fluorophenyl)piperidin-4-yl)methyl)-7-azaspiro[3.5]nonan-2-yl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (Compound 4)

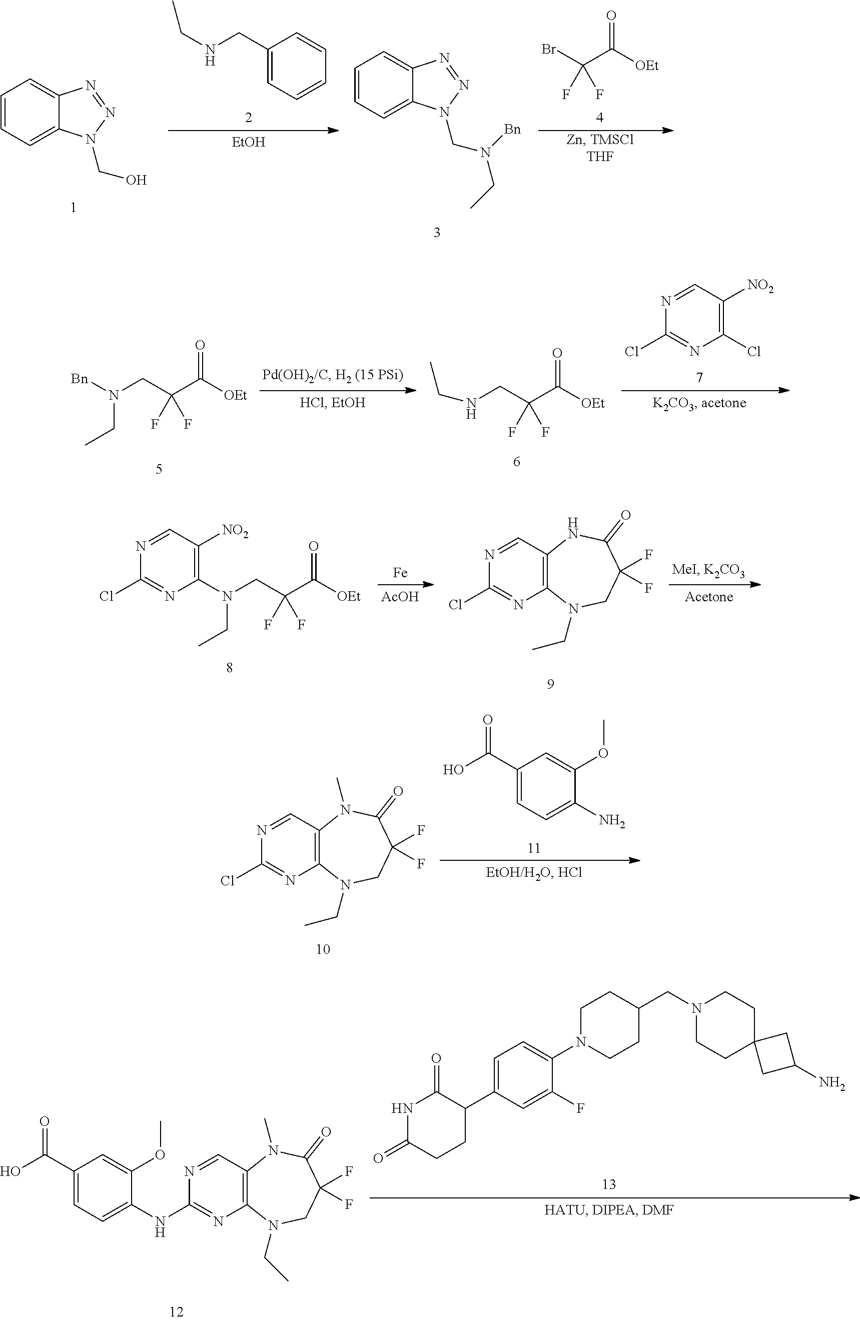

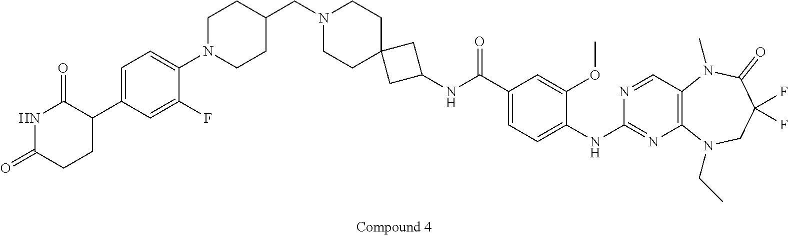 [SMILES: CCN1CC(F)(F)C(=O)N(C)c2cnc(Nc3ccc(C(=O)NC4CC5(CCN(CC6CCN(c7ccc(C8CCC(=O)NC8=O)cc7F)CC6)CC5)C4)cc3OC)nc21]

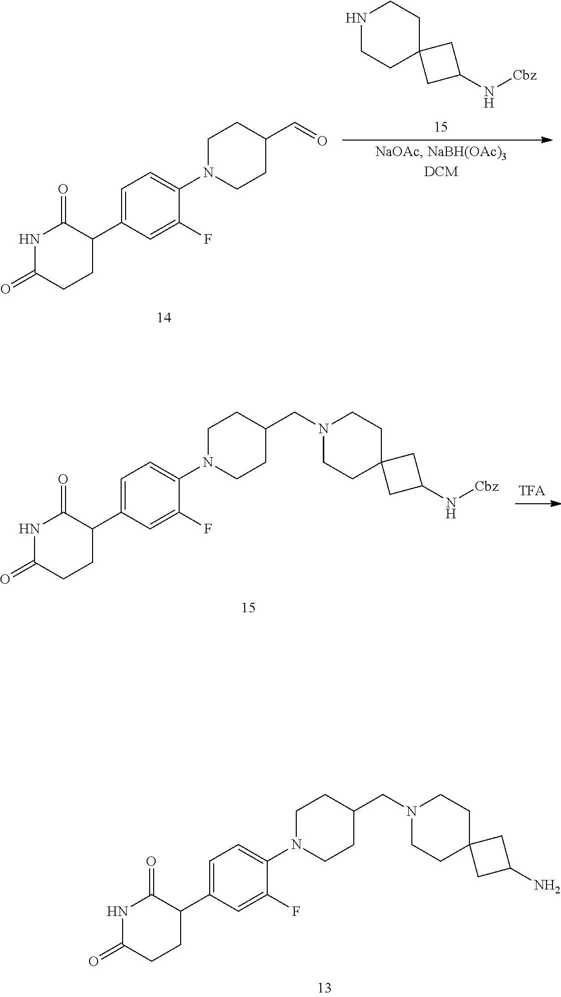 [SMILES: O=C(NC1CC2(CCNCC2)C1)OCc1ccccc1 ; O=CC1CCN(c2ccc(C3CCC(=O)NC3=O)cc2F)CC1 ; O=C1CCC(c2ccc(N3CCC(CN4CCC5(CC4)CC(NC(=O)OCc4ccccc4)C5)CC3)c(F)c2)C(=O)N1 ; NC1CC2(CCN(CC3CCN(c4ccc(C5CCC(=O)NC5=O)cc4F)CC3)CC2)C1]

### Step 1. Synthesis of benzyl (7-((1-(4-(2,6-dioxopiperidin-3-yl)-2-fluorophenyl)piperidin-4-yl)methyl)-7-azaspiro[3.5]nonan-2-yl)carbamate (16)

To a solution of 1-(4-(2,6-dioxopiperidin-3-yl)-2-fluorophenyl)piperidine-4-carbaldehyde (850 mg, 2.67 mmol), benzyl (7-azaspiro[3.5]nonan-2-yl)carbamate (829.91 mg, 2.67 mmol, HCl salt) in DCM (10 mL) was added NaOAc (438.05 mg, 5.34 mmol), the resulting mixture was stirred at 20° C. for 0.5 h. Then NaBH(OAc)₃ (1.70 g, 8.01 mmol) was added and the resulting mixture was stirred at 20° C. for another 16 h. LCMS showed a main peak with desired mass. The mixture was diluted with water (30 mL) extracted with EtOAc (30 mL×3). The combined organic layers were washed with brine (90 mL×2), dried over Na₂SO₄, filtered. The filtrate was concentrated in vacuum. The residue was purified by flash silica gel chromatography (Biotage; 20 g SepaFlash® Silica Flash Column, Eluent of 50-100% EtOAc/Petroleum ether to 10% MeOH/EtOAc gradient @ 100 mL/min) and re-purified by reversed-phase HPLC (column: Phenomenex luna C18 150*40 mm*15 μm; mobile phase: [water (TFA)-ACN]; gradient: 21%-51% B over 10 min) to afford benzyl (7-((1-(4-(2,6-dioxopiperidin-3-yl)-2-fluorophenyl)piperidin-4-yl)methyl)-7-azaspiro[3.5]nonan-2-yl)carbamate (500 mg, 723.88 μmol, 27.11% yield, TFA salt) as a yellow solid. MS(M+H)⁺=577.3

### Step 2. Synthesis of 3-(4-(4-((2-amino-7-azaspiro[3.5]nonan-7-yl)methyl)piperidin-1-yl)-3-fluorophenyl)piperidine-2,6-dione (13)

A mixture of benzyl (7-((1-(4-(2,6-dioxopiperidin-3-yl)-2-fluorophenyl)piperidin-4-yl)methyl)-7-azaspiro[3.5]nonan-2-yl)carbamate (200 mg, 346.80 μmol) in TFA (2 mL) was stirred at 60° C. for 16 h. LCMS showed a main peak with desired mass. The mixture was concentrated in vacuum to afford 3-(4-(4-((2-amino-7-azaspiro[3.5]nonan-7-yl)methyl)piperidin-1-yl)-3-fluorophenyl)piperidine-2,6-dione (200 mg, crude, TFA salt) as a white solid. MS(M+H)⁺=443.2.

### Step 3. Synthesis of N((1H-benzo[d][1,2,3]triazol-1-yl)methyl)-N-benzylethanamine (3)

A mixture of (1H-benzo[d][1,2,3]triazol-1-yl)methanol (2 g, 13.41 mmol) and N-benzylethanamine (1.81 g, 13.41 mmol, 1.99 mL) in EtOH (20 mL) was stirred at 15° C. for 12 h. TLC (Petroleum ether:EtOAc=3:1) indicated the starting material was consumed completely, and one major new spot was detected. The reaction mixture was concentrated under reduced pressure. The residue was purified by flash silica gel chromatography (Biotage; 10 g SepaFlash®Silica Flash Column, Eluent of 0~50% EtOAc/Petroleum ether gradient @ 80 mL/min) to afford N((1H-benzo[d][1,2,3]triazol-1-yl)methyl)-N-benzylethanamine (3.5 g, crude) as a yellow oil. MS(M+H)⁺=267.4.

### Step 4. Synthesis of ethyl 3-(benzyl(ethyl)amino)-2,2-difluoropropanoate (5)

To a suspension of Zn (1.33 g, 20.27 mmol) in THF (60 mL) was added TMSCl (2.08 g, 19.15 mmol, 2.43 mL) dropwise at 0° C. under N₂, during which the temperature was maintained below 5° C. After stirring 20 minutes, ethyl 2-bromo-2,2-difluoroacetate (3.66 g, 18.02 mmol, 2.32 mL) was added slowly at 0° C. under N₂, during which the temperature was maintained below 30° C. The mixture was stirred for 20 minutes and cooled to 0° C. A suspension of N((1H-benzo[d][1,2,3]triazol-1-yl)methyl)-N-benzylethanamine (3 g, 11.26 mmol) in THF (48 mL) was added at 0° C., during which the temperature was maintained below 10° C. After 20 minutes, the suspension was warmed to 15° C. and stirred for 3 hours. LCMS showed a main peak with desired mass. The reaction mixture was filtered and the filter cake was washed with EtOAc (100 mL) and THF (100 mL). The combined filtrates were concentrated in vacuum. The suspension was filtered and the filter cake was washed with the solution (200 mL, Petroleum ether:EtOAc=8:1), the filtrate was concentrated in vacuum to afford ethyl 3-(benzyl(ethyl)amino)-2,2-difluoropropanoate (6 g, crude) as a yellow gum. MS(M+H)⁺=272.3.

### Step 5. Synthesis of ethyl 3-(ethylamino)-2,2-difluoropropanoate (6)

To a solution of ethyl 3-(benzyl(ethyl)amino)-2,2-difluoropropanoate (5 g, 18.43 mmol) and HCl (12 M, 2.00 mL) in EtOH (100 mL) was added Pd(OH)₂ (500.00 mg, 712.08 μmol, 20% purity) under N₂ atmosphere, the suspension was degassed under vacuum and purged with H₂ several times. The mixture was stirred at 25° C. for 64 hours under H₂ (15 psi). LCMS showed a main peak with desired mass. The mixture was filtered through a pad of celite. The filtrate was concentrated in vacuum to afford ethyl 3-(ethylamino)-2,2-difluoropropanoate (5 g, crude, HCl salt) as a yellow oil. MS(M+H)⁺=182.1.

### Step 6. Synthesis of ethyl 3-((2-chloro-5-nitropyrimidin-4-yl)(ethyl)amino)-2,2-difluoropropanoate (8)

To a solution of ethyl 3-(ethylamino)-2,2-difluoropropanoate (5 g, 22.97 mmol, HCl salt) and 2,4-dichloro-5-nitropyrimidine (2.97 g, 15.32 mmol) in acetone (100 mL) was added K₂CO₃ (12.70 g, 91.89 mmol), during which the temperature was maintained 0° C. to 5° C. The suspension was stirred at 15° C. for 16 hours. TLC (Petroleum ether:EtOAc=5:1) showed the starting material was consumed completely. The reaction mixture was diluted with H₂O (100 mL), the resulting mixture was extracted with EtOAc (100 mL×5). The combined organic layers were concentrated in vacuum. The residue was purified by flash silica gel chromatography (Biotage; 20 g SepaFlash®Silica Flash Column, Eluent of 0~12% EtOAc/Petroleum ether gradient @ 100 mL/min) to afford ethyl 3-((2-chloro-5-nitropyrimidin-4-yl)(ethyl)amino)-2,2-difluoropropanoate (3 g, 8.86 mmol, 57.83% yield) as a yellow oil. MS(M+H)⁺=339.7.

### Step 7. Synthesis of 2-chloro-9-ethyl-7,7-difluoro-5,7,8,9-tetrahydro-6H-pyrimido[4,5-b][1,4]diazepin-6-one (9)

To a solution of ethyl 3-((2-chloro-5-nitropyrimidin-4-yl)(ethyl)amino)-2,2-difluoropropanoate (3 g, 8.86 mmol) in AcOH (30 mL) was added Fe (989.30 mg, 17.72 mmol), the mixture was stirred at 20° C. for 1 h, then the suspension was heated to 80° C. for further 16 hours. LCMS showed a main peak with desired mass. The reaction mixture was concentrated in vacuum to remove most of the solvent, the residue was diluted with H₂O (100 mL) and EtOAc (100 mL), the suspension was filtered and the filter cake was washed with EtOAc (200 mL), the organic phase was separated, the aqueous phase was extracted with EtOAc (100 mL×3). The combined organic layer was washed with saturated NaHCO₃ solution (300 mL×2), dried over Na₂SO₄, filtered. The filtrate was concentrated in vacuum. The residue was purified by flash silica gel chromatography (Biotage; 20 g SepaFlash® Silica Flash Column, Eluent of 0~50% EtOAc/Petroleum ether gradient @ 100 mL/min). The eluent was concentrated in vacuum to afford the residue. The residue was triturated with a mixture (Petroleum ether/EtOAc=1/1, 20 mL) at 15° C. for 10 min, the suspension was filtered. The filter cake was washed with a mixture (Petroleum ether/EtOAc=1/1, 100 mL). The filtrate was concentrated in vacuum to afford 2-chloro-9-ethyl-7,7-difluoro-5,7,8,9-tetrahydro-6H-pyrimido[4,5-b][1,4]diazepin-6-on e (1 g, 3.81 mmol, 42.99% yield) as a yellow solid. MS(M+H)⁺=263.0.

### Step 8. Synthesis of 2-chloro-9-ethyl-7,7-difluoro-5-methyl-5,7,8,9-tetrahydro-6H-pyrimido[4,5-b][1,4]diazepin-6-one (10)

To a solution of 2-chloro-9-ethyl-7,7-difluoro-5,7,8,9-tetrahydro-6H-pyrimido[4,5-b][1,4]diazepin-6-on e (900 mg, 3.43 mmol) in ACETONE (10 mL) were added K₂CO₃ (710.41 mg, 5.14 mmol) and Mel (486.39 mg, 3.43 mmol, 213.33 μL), the mixture was stirred at 15° C. for 16 h. LCMS showed a main peak with desired mass. The mixture was filtered through a pad of celite. The filter cake was washed with EtOAc (10 mL). The filtrate was concentrated in vacuum to remove most of the solvent. The residue was diluted with H₂O (20 mL) and extracted with EtOAc (20 mL×3). The combined organic layers were dried over Na₂SO₄, filtered. The filtrate was concentrated in vacuum to afford 2-chloro-9-ethyl-7,7-difluoro-5-methyl-5,7,8,9-tetrahydro-6H-pyrimido[4,5-b][1,4]diazepin-6-one (1 g, crude) as a gray solid. MS(M+H)⁺=277.0.

### Step 9. Synthesis of 4-((9-ethyl-7,7-difluoro-5-methyl-6-oxo-6,7,8,9-tetrahydro-5H-pyrimido[4,5-b][1,4]diazepin-2-yl)amino)-3-methoxybenzoic acid (12)

To a solution of 2-chloro-9-ethyl-7,7-difluoro-5-methyl-5,7,8,9-tetrahydro-6H-pyrimido[4,5-b][1,4]diazepin-6-one (300 mg, 1.08 mmol) and 4-amino-3-methoxybenzoic acid (181.26 mg, 1.08 mmol) in EtOH (2 mL) and H₂O (8 mL) was added HCl (12 M, 135.54 μL), the resulting mixture was stirred at 100° C. for 16 hours. LCMS showed a main peak with desired mass. The mixture was concentrated in vacuum to remove half of the solvent. The residue was filtered. The filter cake was washed with H₂O (10 mL). The filter cake was collected and concentrated in vacuum. The residue was triturated with EtOAc (10 mL) for 30 minutes. The suspension was filtered and the filter cake was washed with EtOAc (10 mL). The filter cake was collected and dried to afford 4-((9-ethyl-7,7-difluoro-5-methyl-6-oxo-6,7,8,9-tetrahydro-5H-pyrimido[4,5-b][1,4]diazepin-2-yl)amino)-3-methoxybenzoic acid (300 mg, 736.43 μmol, 67.92% yield) as a gray solid. MS(M+H)⁺=408.1.

### Step 10. Synthesis of N(7-((1-(4-(2,6-dioxopiperidin-3-yl)-2-fluorophenyl)piperidin-4-yl)methyl)-7-azaspiro[3.5]nonan-2-yl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (Compound 4)

It was synthesized by similar method described in step 10 of Example 2 to obtain N(7-((1-(4-(2,6-dioxopiperidin-3-yl)-2-fluorophenyl)piperidin-4-yl)methyl)-7-azaspiro[3.5]nonan-2-yl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (47.3 mg, 54.58 μmol, 31.76% yield, 96% purity) as a white solid. MS(M+H)⁺=832.4.

¹H NMR (400 MHz, DMSO-d₆) δ=10.81 (s, 1H), 8.48-8.39 (m, 1H), 8.31 (d, J=8.4 Hz, 1H), 8.23 (s, 1H), 7.90 (s, 1H), 7.53-7.44 (m, 2H), 7.05-6.92 (m, 3H), 4.49-4.34 (m, 1H), 4.17 (t, J=13.7 Hz, 2H), 3.94 (s, 3H), 3.84-3.75 (m, 1H), 3.70-3.59 (m, 2H), 3.50-3.33 (m, 5H), 2.72-2.56 (m, 6H), 2.49-2.42 (m, 2H), 2.26-2.10 (m, 5H), 2.04-1.94 (m, 2H), 1.88-1.74 (m, 5H), 1.66-1.56 (m, 2H), 1.35-1.15 (m, 6H).

### Example 5. Synthesis of 4-((7,7-difluoro-5,9-dimethyl-6-oxo-6,7,8,9-tetrahydro-5H-pyrimido[4,5-b][1,4]diazepin-2-yl)amino)-N-(7-((1-(4-(2,6-dioxopiperidin-3-yl)-2-fluorophenyl)piperidin-4-yl)methyl)-7-azaspiro[3.5]nonan-2-yl)-3-methoxybenzamide (Compound 5)

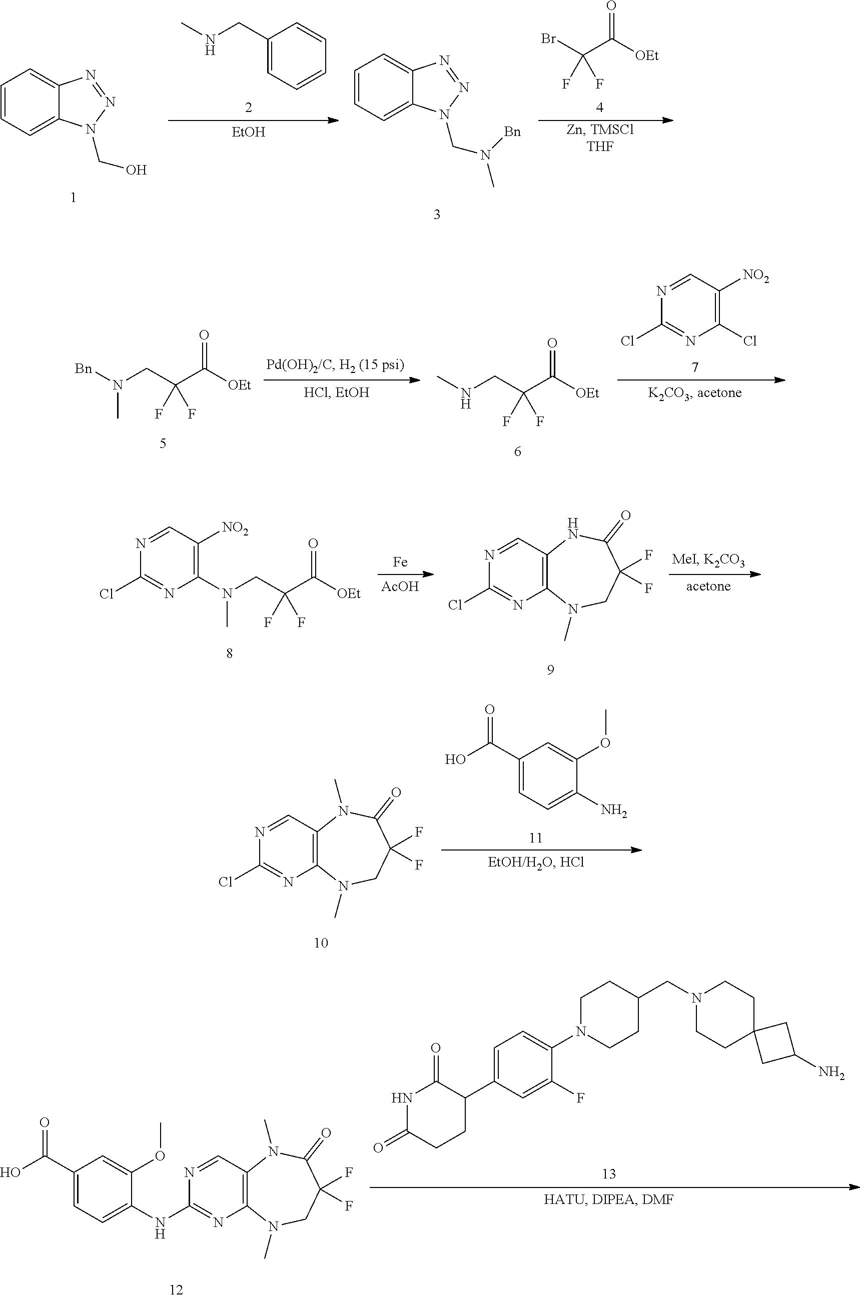

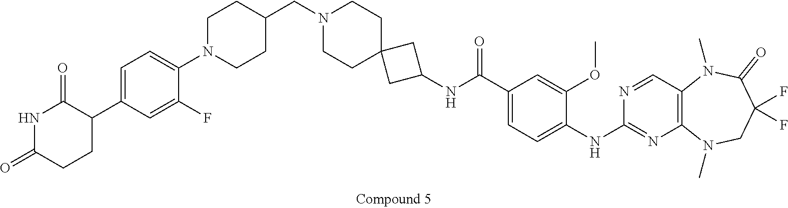 [SMILES: COc1cc(C(=O)NC2CC3(CCN(CC4CCN(c5ccc(C6CCC(=O)NC6=O)cc5F)CC4)CC3)C2)ccc1Nc1ncc2c(n1)N(C)CC(F)(F)C(=O)N2C]

It was synthesized by similar method described in Example 4 to obtain 4-((7,7-difluoro-5,9-dimethyl-6-oxo-6,7,8,9-tetrahydro-5H-pyrimido[4,5-b][1,4]diazepin-2-yl)amino)-N-(7-((1-(4-(2,6-dioxopiperidin-3-yl)-2-fluorophenyl)piperidin-4-yl)methyl)-7-azaspiro[3.5]nonan-2-yl)-3-methoxybenzamide (52.5 mg, 60.34 mol, 33.90% yield, 94% purity) as a white solid. MS(M+H)⁺=818.4.

¹H NMR (400 MHz, DMSO-d₆) δ=10.81 (s, 1H), 8.44 (d, J=7.2 Hz, 1H), 8.36 (d, J=8.4 Hz, 1H), 8.29 (s, 1H), 7.96 (s, 1H), 7.56-7.46 (m, 2H), 7.05-6.90 (m, 3H), 4.45-4.36 (m, 1H), 4.17 (t, J=14.4 Hz, 2H), 3.94 (s, 3H), 3.83-3.76 (m, 1H), 3.36-3.32 (m, 5H), 3.06 (s, 3H), 2.72-2.57 (m, 4H), 2.49-2.41 (m, 3H), 2.29-2.12 (m, 5H), 2.02-1.96 (m, 1H), 1.89-1.73 (m, 5H), 1.71-1.59 (m, 4H), 1.33-1.21 (m, 3H).

### Example 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(4-(2,6-dioxopiperidin-3-yl)-2-meth ylphenyl)piperidin-4-yl)methyl)(methyl)amino)cyclohexyl)-3-methoxybenzamide (Compound 6)

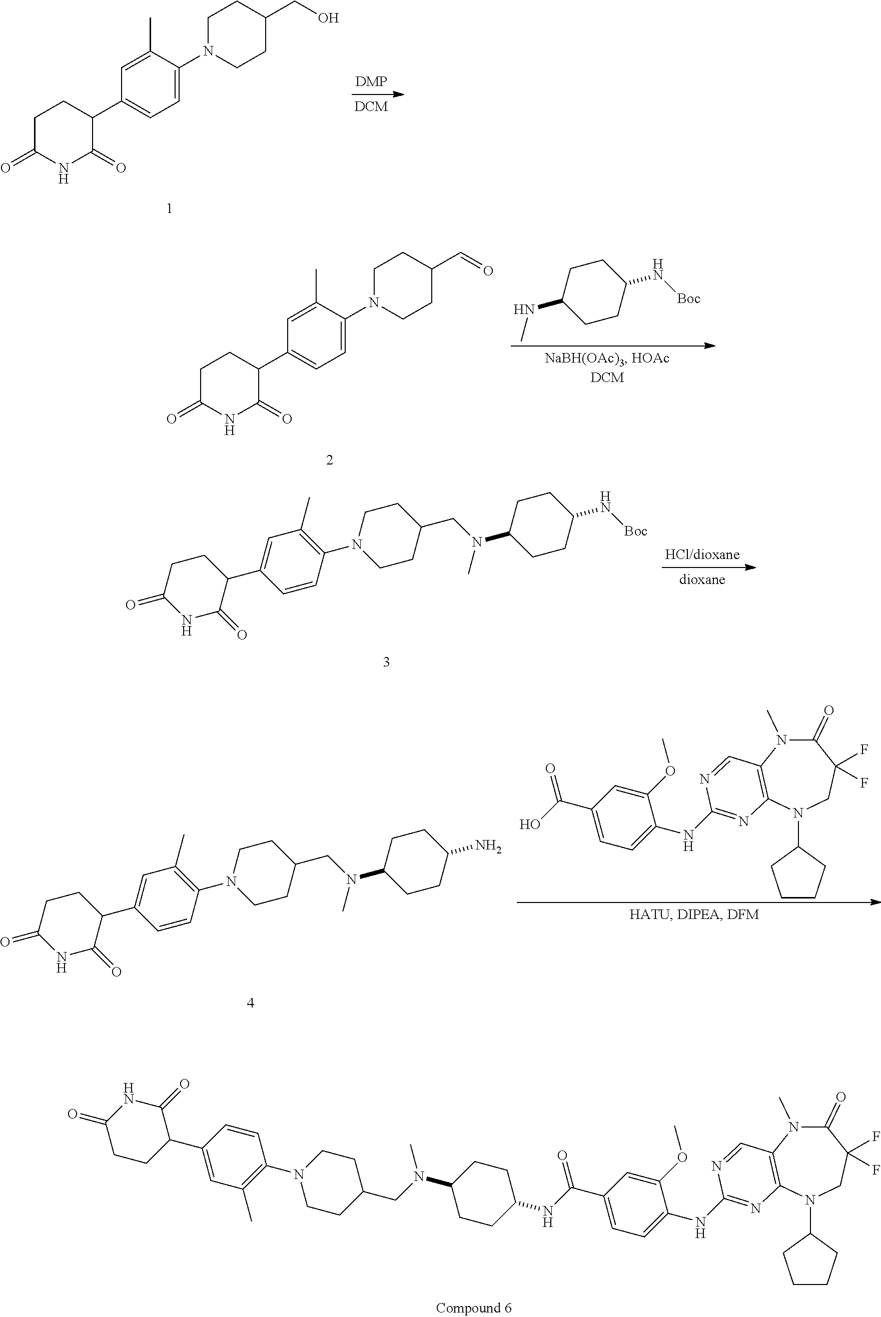

### Step 1. Synthesis of 1-(4-(2,6-dioxopiperidin-3-yl)-2-methylphenyl) piperidine-4-carbaldehyde (2)

To a solution of 3-(4-(4-(hydroxymethyl) piperidin-1-yl)-3-methylphenyl) piperidine-2,6-dione (0.5 g, 1.58 mmol) in DCM (30 mL) was added DMP (1.01 g, 2.37 mmol) and the mixture was stirred at 25° C. for 2 h. TLC (Petroleum ether:EtOAc=1:1) showed that 3-(4-(4-(hydroxymethyl) piperidin-1-yl)-3-methylphenyl) piperidine-2,6-dione was consumed completely and new spot was formed. The mixture was filtered and the filtrate was concentrated to afford 1-(4-(2,6-dioxopiperidin-3-yl)-2-methylphenyl)piperidine-4-carbaldehyde (0.5 g, crude) as a yellow oil. MS(M+H)⁺=315.1.

### Step 2. Synthesis of tert-butyl ((1r,4r)-4-(((1-(4-(2,6-dioxopiperidin-3-yl)-2-methylphenyl)piperidin-4-yl)methyl)(methyl)amino)cyclohexyl)carbamate (3)

To the solution of 1-(4-(2,6-dioxopiperidin-3-yl)-2-methylphenyl)piperidine-4-carbaldehyde (0.5 g, 1.59 mmol) and tert-butyl ((1r,4r)-4-(methylamino)cyclohexyl)carbamate (363.15 mg, 1.59 mmol) in DCM (10 mL) were added NaBH(OAc)₃ (505.62 mg, 2.39 mmol) and AcOH (105.06 mg, 1.75 mmol, 100.06 μL) and the resulting mixture was stirred at 25° C. for 12 h. LCMS showed a main peak (77%) with desired mass. The mixture was poured into water (20 mL) and extracted with DCM (20 mL×3). The combined organic layer was washed by brine (50 mL), dried over Na₂SO₄, filtered and concentrated. The residue was purified by flash silica gel chromatography (20 g SepaFlash® Silica Flash Column, Eluent of 0-30% MeOH/EtOAc gradient @ 80 mL/min) to afford tert-butyl ((1r,4r)-4-(((1-(4-(2,6-dioxopiperidin-3-yl)-2-methylphenyl)piperidin-4-yl)methyl)(methyl)amino)cyclohexyl)carbamate (0.27 g, 512.62 μmol, 32.23% yield) as a yellow solid. MS(M+H)⁺=527.5.

### Step 3. Synthesis of 3-(4-(4-((((1r,4r)-4-aminocyclohexyl)(methyl)amino)methyl)piperidin-1-yl)-3-meth ylphenyl)piperidine-2,6-dione (4)

It was synthesized by similar method described in step 5 of Example 1 to obtain 3-(4-(4-((((1r,4r)-4-aminocyclohexyl)(methyl)amino)methyl)piperidin-1-yl)-3-methylphenyl)piperidine-2,6-dione (0.2 g, crude, HCl salt) as a yellow oil. MS(M+H)⁺=427.3.

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(4-(2,6-dioxopiperidin-3-yl)-2-meth ylphenyl)piperidin-4-yl)methyl)(methyl)amino)cyclohexyl)-3-methoxybenzamide (Compound 6)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(4- (2,6-dioxopiperidin-3-yl)-2-methylphen yl)piperidin-4-yl)methyl)(methyl)amino)cyclohexyl)-3-methoxybenzamide (33 mg, 37.39 μmol, 17.32% yield, 97% purity) as a white solid. MS(M+H)⁺=856.4.

¹H NMR (400 MHz, DMSO-d₆) δ=10.79 (s, 1H), 8.30-8.24 (m, 2H), 8.09 (br d, J=8.1 Hz, 1H), 7.96 (s, 1H), 7.54-7.44 (m, 2H), 7.04-6.92 (m, 3H), 4.87-4.69 (m, 1H), 4.17-4.00 (m, 2H), 3.94 (s, 3H), 3.74 (br dd, J=5.1, 11.1 Hz, 2H), 3.55-3.37 (m, 1H), 3.33 (s, 3H), 3.10-3.02 (m, 2H), 2.70-2.53 (m, 4H), 2.47-2.42 (m, 1H), 2.38-2.26 (m, 3H), 2.28-2.19 (m, 4H), 2.15-2.01 (m, 2H), 2.01-1.89 (m, 5H), 1.87-1.66 (m, 6H), 1.65-1.57 (m, 4H), 1.48-1.39 (m, 4H), 1.34-1.26 (m, 2H).

### Example 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-(2,6-dioxopiperidin-3-yl)-2-fluoro-5-meth oxyphenyl)piperazin-1-yl)ethyl)piperidin-1-yl)-3-methoxybenzamide (Compound 7)

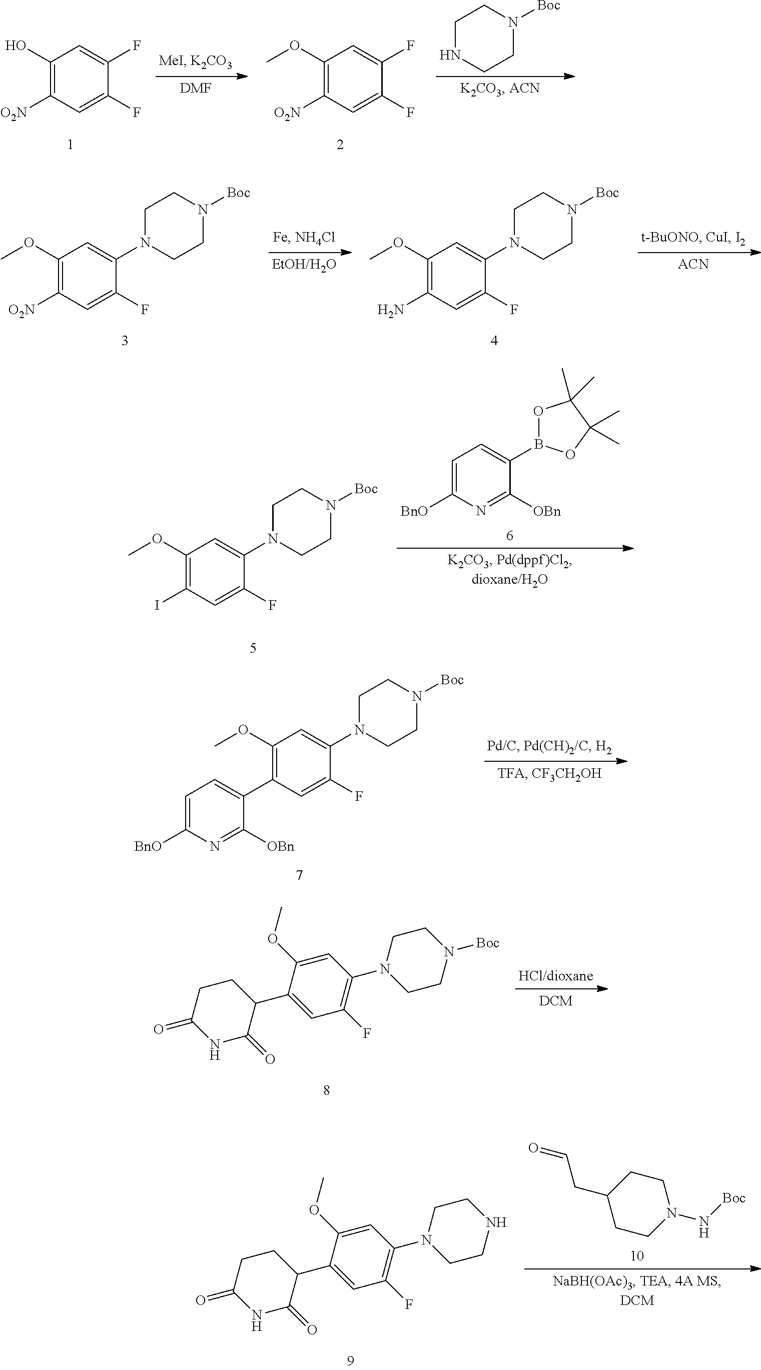

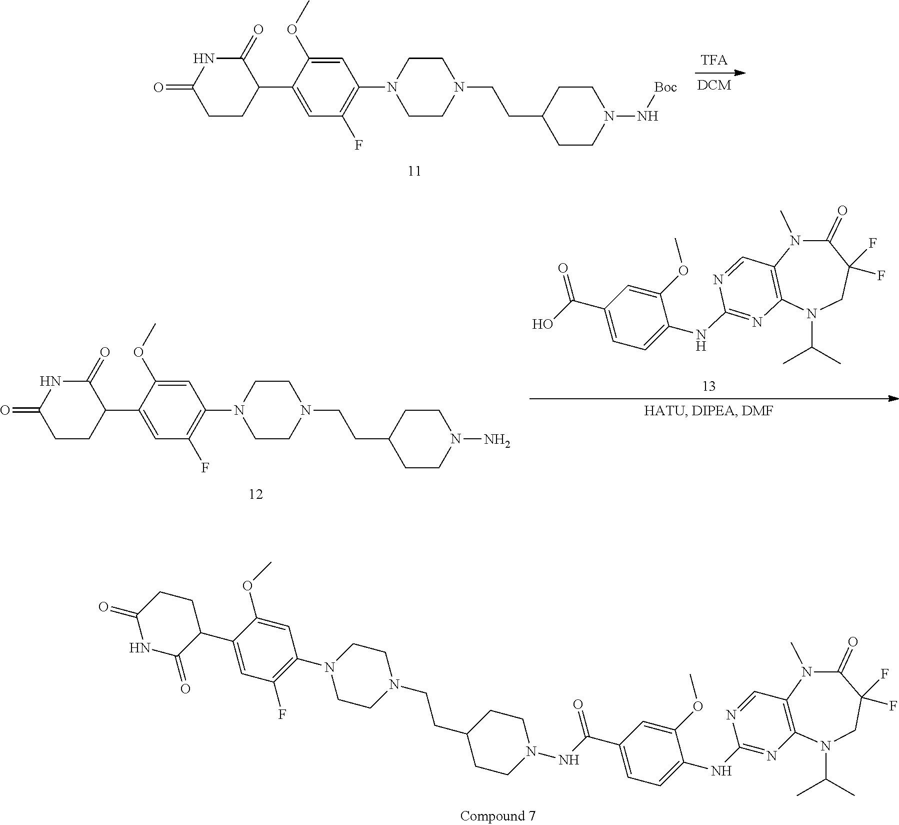 [SMILES: COc1cc(N2CCN(CCC3CCN(NC(=O)OC(C)(C)C)CC3)CC2)c(F)cc1C1CCC(=O)NC1=O ; COc1cc(N2CCN(CCC3CCN(N)CC3)CC2)c(F)cc1C1CCC(=O)NC1=O ; COc1cc(C(=O)O)ccc1Nc1ncc2c(n1)N(C(C)C)CC(F)(F)C(=O)N2C ; COc1cc(C(=O)NN2CCC(CCN3CCN(c4cc(OC)c(C5CCC(=O)NC5=O)cc4F)CC3)CC2)ccc1Nc1ncc2c(n1)N(C(C)C)CC(F)(F)C(=O)N2C]

### Step 1. Synthesis of 1,2-difluoro-4-methoxy-5-nitrobenzene (2)

It was synthesized by similar method described in step 8 of Example 4, changed Acetone to DMF (30 mL) to obtain 1,2-difluoro-4-methoxy-5-nitrobenzene (3.9 g, crude) as yellow oil. MS(M+H)⁺=190.2

### Step 2. Synthesis of tert-butyl 4-(2-fluoro-5-methoxy-4-nitrophenyl)piperazine-1-carboxylate (3)

It was synthesized by similar method described in step 6 of Example 4, changed Acetone to ACN to obtain tert-butyl 4-(2-fluoro-5-methoxy-4-nitrophenyl)piperazine-1-carboxylate (4.3 g, 12.10 mmol, 58.68% yield) as a yellow solid. MS(M-56+H)⁺=300.1

### Step 3. Synthesis of tert-butyl 4-(4-amino-2-fluoro-5-methoxyphenyl)piperazine-1-carboxylate (4)

It was synthesized by similar method described in step 9 of Example 2, changed Zn, THF to Fe(3.38 g, 60.50 mmol), EtOH (60 mL) to obtain tert-butyl 4-(4-amino-2-fluoro-5-methoxyphenyl)piperazine-1-carboxylate (4 g, 11.80 mmol, 97.53% yield, 96% purity) as a brown solid. MS(M+H)⁺=326.1

### Step 4. Synthesis of tert-butyl 4-(2-fluoro-4-iodo-5-methoxyphenyl)piperazine-1-carboxylate (5)

To the solution of tert-butyl nitrite (1.27 g, 12.29 mmol, 1.46 mL) in ACN (40 mL) were added tert-butyl 4-(4-amino-2-fluoro-5-methoxyphenyl)piperazine-1-carboxylate (2 g, 6.15 mmol), I₂ (3.12 g, 12.29 mmol) and CuI (2.34 g, 12.29 mmol) at 0° C. and the resulting mixture was stirred at 20° C. for 16 h. LCMS showed a peak (23%) with desired mass. The reaction was filtered, then the filtrate was concentrated. The residue was diluted with water (20 mL) and extracted with EtOAc (20 mL×3). The combined organic layers were washed with saturated brine (50 mL), dried over anhydrous Na₂SO 4, filtered and concentrated under reduced pressure. The residue was purified by flash silica gel chromatography (ISCO®; 10 g SepaFlash® Silica Flash Column, Eluent of 0~30% EtOAc/Petroleum ether gradient @ 80 mL/min) to afford tert-butyl 4-(2-fluoro-4-iodo-5-methoxyphenyl)piperazine-1-carboxylate (560 mg, 1.17 mmol, 19.00% yield, 91% purity) as yellow oil. MS(M+H)⁺=437.0

### Step 5. Synthesis of tert-butyl 4-(4-(2,6-bis(benzyloxy)pyridin-3-yl)-2-fluoro-5-methoxyphenyl)piperazine-1-carboxylate (7)

It was synthesized by similar method described in step 4 of Example 3 to obtain tert-butyl 4-(4-(2,6-bis(benzyloxy)pyridin-3-yl)-2-fluoro-5-methoxyphenyl)piperazine-1-carboxy late (570 mg, 845.94 μmol, 65.90% yield, 89% purity) as yellow oil. MS(M+H)⁺=600.3

### Step 6. Synthesis of tert-butyl 4-(4-(2,6-dioxopiperidin-3-yl)-2-fluoro-5-methoxyphenyl)piperazine-1-carboxylate (8)

It was synthesized by similar method described in step 2 of Example 3, changed EtOH, AcOH to CF₃CH₂OH (20 mL), TFA (47.53 mg, 416.88 μmol, 30.97 μL) to obtain tert-butyl 4-(4-(2,6-dioxopiperidin-3-yl)-2-fluoro-5-methoxyphenyl)piperazine-1-carboxylate (0.2 g, 373.49 μmol, 89.59% yield, TFA salt) as a brown solid. MS(M+H+Na)⁺=444.3

### Step 7. Synthesis of 3-(5-fluoro-2-methoxy-4-(piperazin-1-yl)phenyl)piperidine-2,6-dione (9)

It was synthesized by similar method described in step 5 of Example 1, changed dioxane to DCM (2 mL) to obtain 3-(5-fluoro-2-methoxy-4-(piperazin-1-yl)phenyl)piperidine-2,6-dione (0.17 g, crude, HCl) as a white solid. MS(M+H)⁺=322.2

### Step 8. Synthesis of tert-butyl (4-(2-(4-(4-(2,6-dioxopiperidin-3-yl)-2-fluoro-5-methoxyphenyl)piperazin-1-yl)eth yl)piperidin-1-yl)carbamate (11)

It was synthesized by similar method described in step 6 of Example 1 to obtain tert-butyl (4-(2-(4-(4-(2,6-dioxopiperidin-3-yl)-2-fluoro-5-methoxyphenyl)piperazin-1-yl)ethyl)piperidin-1-yl)carbamate (60 mg, 93.12 mol, 19.95% yield, 85% purity) as a yellow solid. MS(M+H)⁺=548.3

### Step 9. Synthesis of 3-(4-(4-(2-(1-aminopiperidin-4-yl)ethyl)piperazin-1-yl)-5-fluoro-2-methoxyphenyl) piperidine-2,6-dione (12)

It was synthesized by similar method described in step 6 of Example 3 to obtain 3-(4-(4-(2-(1-aminopiperidin-4-yl)ethyl)piperazin-1-yl)-5-fluoro-2-methoxyphenyl)piperidine-2,6-dione (50 mg, 89.04 mol, 97.52% yield, TFA) as brown oil. MS(M+H)⁺=448.3

### Step 10. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-(2,6-dioxopiperidin-3-yl)-2-fluoro-5-meth oxyphenyl)piperazin-1-yl)ethyl)piperidin-1-yl)-3-methoxybenzamide (Compound 7)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-(2,6-dioxopiperidin-3-yl)-2-fluoro-5-methoxyphenyl)piperazin-1-yl)ethyl)piperidin-1-yl)-3-methoxybenzamide (36.6 mg, 36.03 mol, 40.47% yield, 95% purity, TFA) as a white solid. MS(M+H)⁺=851.6.

¹H NMR (400 MHz, DMSO-d₆) δ=10.74 (s, 1H), 9.53 (br s, 1H), 8.28 (d, J=8.4 Hz, 1H), 8.23 (s, 1H), 8.16-8.03 (m, 1H), 7.50-7.37 (m, 2H), 7.04 (d, J=13.1 Hz, 1H), 6.67 (d, J=7.5 Hz, 1H), 4.99-4.81 (m, 1H), 4.08 (s, 2H), 3.94 (s, 3H), 3.95-3.86 (m, 4H), 3.63-3.57 (m, 4H), 3.33 (s, 3H), 3.28-3.18 (m, 4H), 3.14-3.04 (m, 4H), 2.87-2.78 (m, 2H), 2.62-2.57 (m, 2H), 2.28-2.12 (m, 1H), 1.92-1.83 (m, 1H), 1.81-1.73 (m, 2H), 1.71-1.62 (m, 2H), 1.45-1.36 (m, 3H), 1.25 (d, J=6.6 Hz, 6H).

### Example 8. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(3-(2,4-dioxotetrahydropyrimidin-1 (2H)-yl)phenyl)piperidin-4-yl)methyl)(methyl)amino)cyclohexyl)-3-methoxybenzamide (Compound 8)

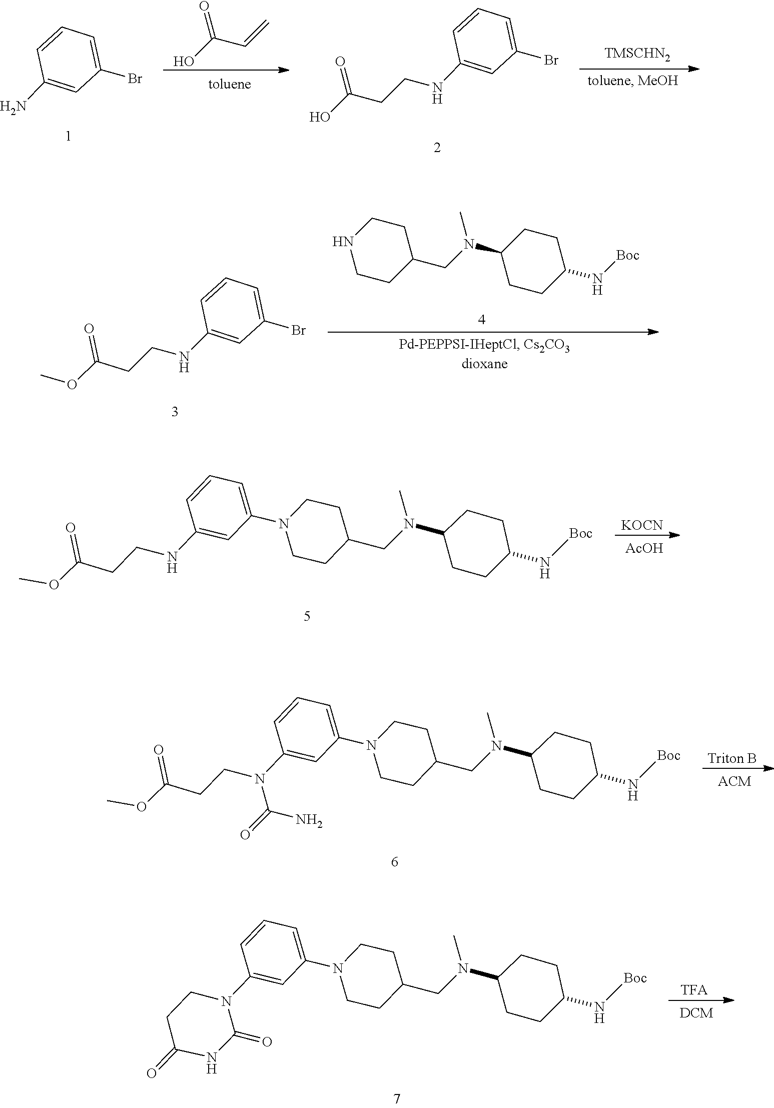

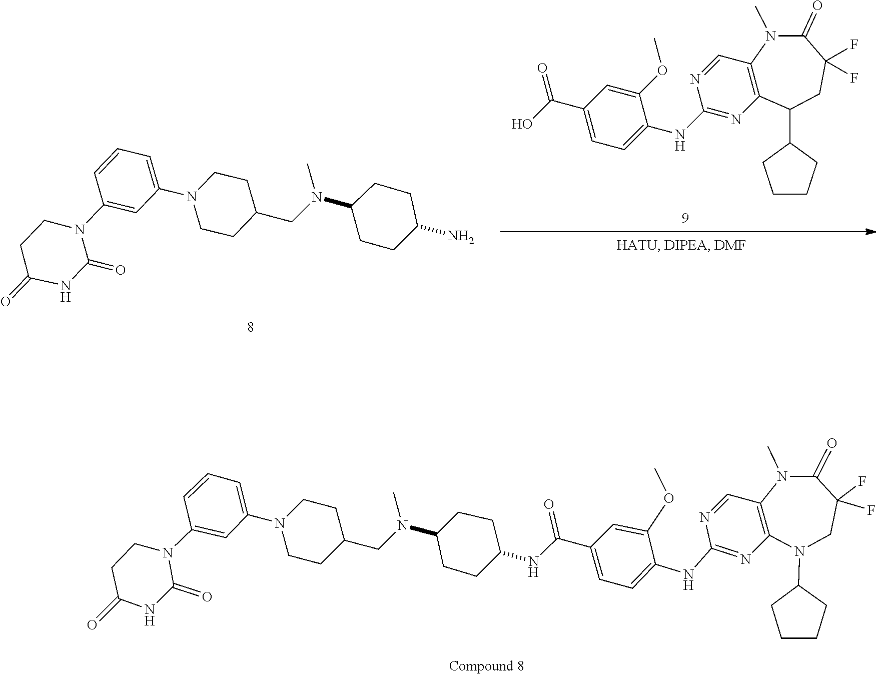 [SMILES: CN(CC1CCN(c2cccc(N3CCC(=O)NC3=O)c2)CC1)[C@H]1CC[C@H](N)CC1 ; COc1cc(C(=O)O)ccc1Nc1ncc2c(n1)C(C1CCCC1)CC(F)(F)C(=O)N2C ; COc1cc(C(=O)N[C@H]2CC[C@H](N(C)CC3CCN(c4cccc(N5CCC(=O)NC5=O)c4)CC3)CC2)ccc1Nc1ncc2c(n1)N(C1CCCC1)CC(F)(F)C(=O)N2C]

### Step 1. Synthesis of 3-((3-bromophenyl)amino)propanoic acid (2)

To a solution of 3-bromoaniline (1 g, 5.81 mmol, 632.91 μL) in toluene (10 mL) was added acrylic acid (630.00 mg, 8.74 mmol, 0.6 mL). The mixture was stirred at 80° C. for 14 h. LCMS showed 96% of the desired mass. The mixture was concentrated under reduced pressure. The residue was purified by flash silica gel chromatography (12 g SepaFlash® Silica Flash Column, Eluent of 20-40% EtOAc/Petroleum ether gradient @ 100 mL/min) to afford 3-((3-bromophenyl)amino)propanoic acid (890 mg, 3.50 mmol, 60.22% yield, 96% purity) as a yellow oil. MS(M+H)⁺=244.2.

### Step 2. Synthesis of methyl 3-((3-bromophenyl)amino)propanoate (3)

To a solution of 3-((3-bromophenyl)amino)propanoic acid (890 mg, 3.65 mmol) in toluene (8 mL) and MeOH (8 mL) was added TMSCHN₂ (2 M, 5.5 mL) slowly and the mixture was stirred at 25° C. for 14 h. LCMS showed 41% of the starting material remained and 34% of the desired mass. The mixture was concentrated under reduced pressure. The residue was purified by flash silica gel chromatography (12 g SepaFlash® Silica Flash Column, Eluent of 10-50% EtOAc/Petroleum ether gradient @ 100 mL/min) to afford methyl 3-((3-bromophenyl)amino)propanoate (340 mg, 1.28 mmol, 35.04% yield, 97% purity) as a yellow oil. MS(M+H)⁺=258.1.

### Step 3. Synthesis of methyl 3-((3-(4-((((1r,4r)-4-((tert-butoxycarbonyl)amino)cyclohexyl)(methyl)amino)meth yl)piperidin-1-yl)phenyl)amino)propanoate (5)

To a solution of tert-butyl ((1r,4r)-4-(methyl(piperidin-4-ylmethyl)amino)cyclohexyl)carbamate (396 mg, 1.22 mmol) and methyl 3-((3-bromophenyl)amino)propanoate (0.24 g, 929.83 μmol) in dioxane (5 mL) were added Cs₂CO₃ (607 mg, 1.86 mmol) and Pd-PEPPSI-IHeptCl (63.32 mg, 65.09 μmol) and the mixture was stirred at 100° C. for 14 h. LCMS showed the desired mass. The mixture was filtered and the filter cake was washed with EtOAc (20 mL). The filtrate was concentrated under reduced pressure. The residue was purified by flash silica gel chromatography (5 g SepaFlash® Silica Flash Column, Eluent of 80-100% EtOAc/Petroleum ether to 10% MeOH/EtOAc gradient @ 100 mL/min), then re-purified by prep-TLC (DCM:MeOH=10:1) to afford methyl 3-((3-(4-((((1r,4r)-4-((tert-butoxycarbonyl)amino)cyclohexyl)(methyl)amino)methyl)piperidin-1-yl)phenyl)amino)propanoate (150 mg, 298.40 mol, 32.09% yield) as a yellow oil. MS(M+H)⁺=503.5.

### Step 4. Synthesis of methyl 3-(1-(3-(4-((((1r,4r)-4-((tert-butoxycarbonyl)amino)cyclohexyl)(methyl)amino)methyl)piperidin-1-yl)phenyl)ureido)propanoate (6)

To a solution of methyl 3-((3-(4-((((1r,4r)-4-((tert-butoxycarbonyl)amino)cyclohexyl)(methyl)amino)methyl)piperidin-1-yl)phenyl)amino)propanoate (120 mg, 238.72 μmol) in AcOH (1 mL) was added KOCN (30.5 mg, 358.19 mol) and the mixture was stirred at 25° C. for 14 h. LCMS showed 80% of the desired mass. The mixture was concentrated under reduced pressure. The crude was diluted with MeCN (3 mL) and water (30 mL), then the suspension was lyophilized to afford methyl 3-(1-(3-(4-((((1r,4r)-4-((tert-butoxycarbonyl)amino)cyclohexyl)(methyl)amino)methyl) piperidin-1-yl)phenyl)ureido)propanoate (150 mg, crude, AcOH salt) as a yellow solid. MS(M+H)⁺=546.6.

### Step 5. Synthesis of tert-butyl ((1r,4r)-4-(((1-(3-(2,4-dioxotetrahydropyrimidin-1(2H)-yl)phenyl)piperidin-4-yl)methyl)(methyl)amino)cyclohexyl)carbamate (7)

To a solution of methyl 3-(1-(3-(4-((((1r,4r)-4-((tert-butoxycarbonyl)amino)cyclohexyl)(methyl)amino)methyl) piperidin-1-yl)phenyl)ureido)propanoate (120 mg, 198.10 mol, AcOH salt) in ACN (2.5 mL) was added Triton B (552.00 mg, 1.32 mmol, 600 μL, 40% purity) slowly and the mixture was stirred at 25° C. for 1 h. LCMS showed 61% of the desired mass. The mixture was diluted with water (10 mL) and extracted with EtOAc (10 mL×3), the combined organic layer was washed with water (10 mL), dried over Na₂SO₄ and filtered. The filtrate was concentrated under reduced pressure to afford tert-butyl ((1r,4r)-4-(((1-(3-(2,4-dioxotetrahydropyrimidin-1(2H)-yl)phenyl)piperidin-4-yl)methy 1)(methyl)amino)cyclohexyl)carbamate (90 mg, crude) as a yellow solid. MS(M+H)⁺=514.5.

### Step 6. Synthesis of 1-(3-(4-((((1r,4r)-4-aminocyclohexyl)(methyl)amino)methyl)piperidin-1-yl)phenyl) dihydropyrimidine-2,4(1H,3H)-dione (8)

It was synthesized by similar method described in step 6 of Example 3 to obtain 1-(3-(4-((((1r,4r)-4-aminocyclohexyl)(methyl)amino)methyl)piperidin-1-yl)phenyl)dihydropyrimidine-2,4(1H,3H)-dione (90 mg, crude, TFA) as a yellow oil. MS(M+H)⁺=414.4.

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(3-(2,4-dioxotetrahydropyrimidin-1 (2H)-yl)phenyl)piperidin-4-yl)methyl)(methyl)amino)cyclohexyl)-3-methoxybenzamide (Compound 8)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(3-(2,4-dioxotetrahydropyrimidin-1(2H)-yl) phenyl)piperidin-4-yl)methyl)(methyl)amino)cyclohexyl)-3-methoxybenzamide (48.8 mg, 56.73 μmol, 36.26% yield, 98% purity) as a white solid. MS(M+H)⁺=843.4.

¹H NMR (400 MHz, CD₃CN) δ=8.47 (d, J=8.3 Hz, 1H), 8.30-8.19 (m, 1H), 8.10 (s, 1H), 7.70 (s, 1H), 7.44-7.37 (m, 2H), 7.24 (t, J=8.1 Hz, 1H), 6.91-6.82 (m, 3H), 6.75-6.69 (m, 1H), 4.92-4.80 (m, 1H), 4.01-3.92 (m, 5H), 3.89-3.82 (m, 1H), 3.77 (t, J=6.7 Hz, 2H), 3.72 (br d, J=12.5 Hz, 2H), 3.33 (s, 3H), 3.11-3.02 (m, 1H), 2.86-2.81 (m, 2H), 2.78-2.73 (m, 2H), 2.72-2.69 (m, 2H), 2.64 (s, 3H), 2.15-2.08 (m, 2H), 2.06-1.99 (m, 3H), 1.89-1.82 (m, 2H), 1.79-1.73 (m, 2H), 1.69-1.57 (m, 7H), 1.52-1.40 (m, 2H), 1.40-1.26 (m, 3H).

### Example 9. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)pyridin-3-yl)piperidin-4-yl)(methyl)amino)methyl)cyclohexyl)-3-methoxybenzamide (Compound 9)

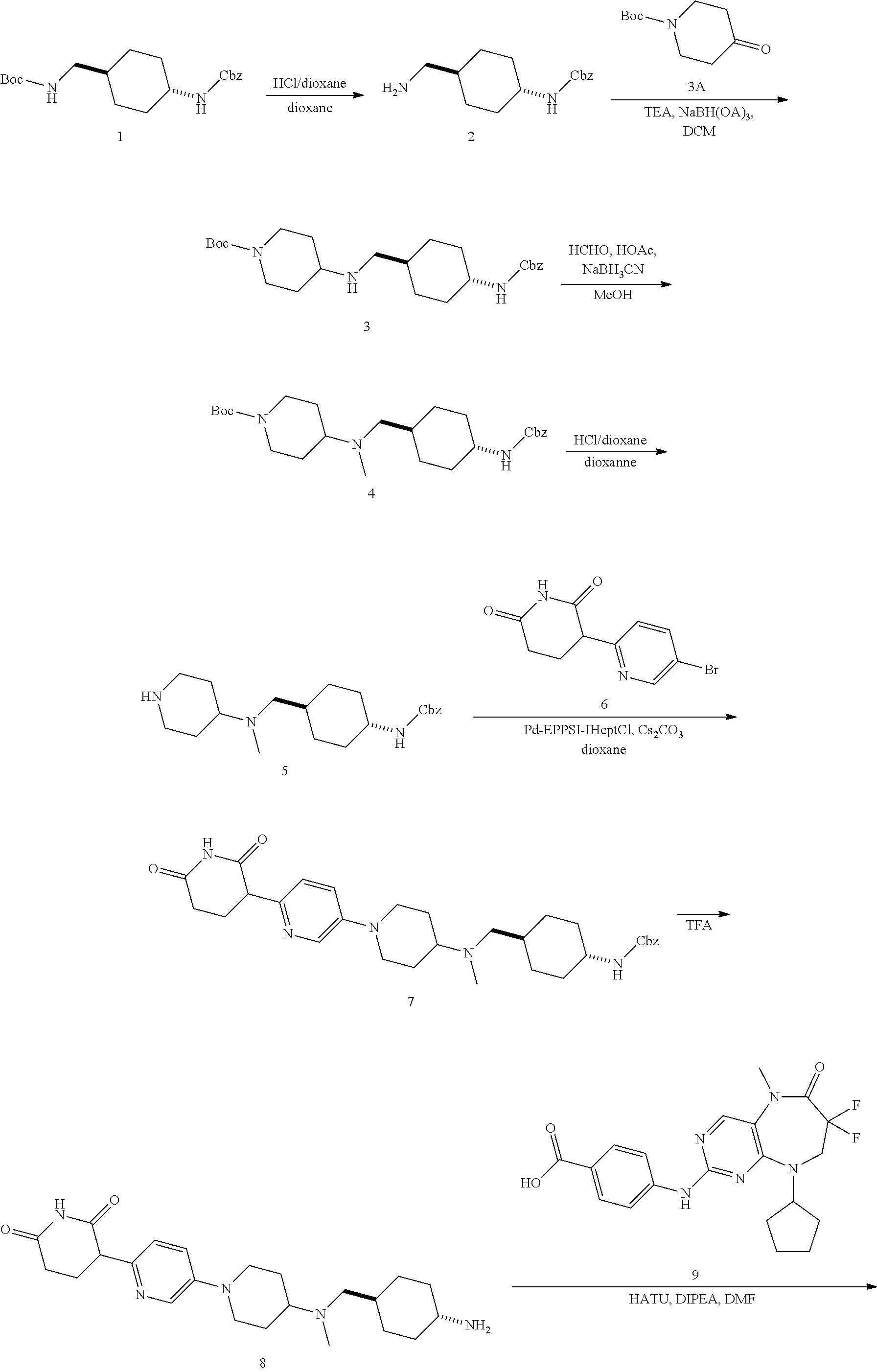

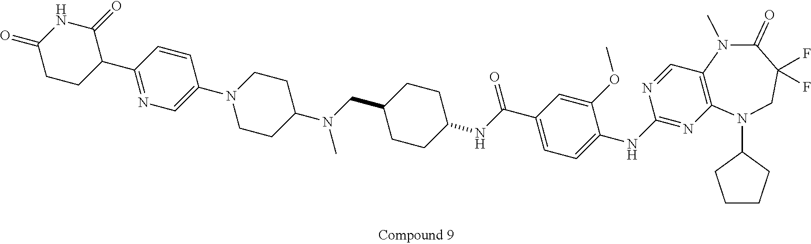 [SMILES: COc1cc(C(=O)N[C@H]2CC[C@H](CN(C)C3CCN(c4ccc(C5CCC(=O)NC5=O)nc4)CC3)CC2)ccc1Nc1ncc2c(n1)N(C1CCCC1)CC(F)(F)C(=O)N2C]

### Step 1. Synthesis of benzyl ((1r,4r)-4-(aminomethyl)cyclohexyl)carbamate (2)

It was synthesized by similar method described in step 5 of Example 1 to obtain benzyl ((1r,4r)-4-(aminomethyl)cyclohexyl)carbamate (410 mg, crude, HCl salt) as a light yellow solid. MS(M+H)⁺=263.2.

### Step 2. Synthesis of tert-butyl 4-((((1r,4r)-4-(((benzyloxy)carbonyl)amino)cyclohexyl)methyl)amino)piperidine-1-carboxylate (3)

It was synthesized by similar method described in step 6 of Example 1 to obtain tert-butyl 4-((((1r,4r)-4-(((benzyloxy)carbonyl)amino)cyclohexyl)methyl)amino)piperidine-1-carboxylate (670 mg, crude) as a yellow solid. MS(M+H)⁺=446.3.

### Step 3. Synthesis of tert-butyl 4-((((1r,4r)-4-(((benzyloxy)carbonyl)amino)cyclohexyl)methyl)(methyl)amino)piperidine-1-carboxylate (4)

To a solution of tert-butyl 4-((((1r,4r)-4-(((benzyloxy)carbonyl)amino)cyclohexyl)methyl)amino)piperidine-1-carboxylate (670 mg, 1.50 mmol, crude) in MeOH (30 mL) was added AcOH (90.29 mg, 1.50 mmol, 86.00 μL) and HCHO (1.22 g, 15.04 mmol, 1.12 mL, 37% purity). The mixture was stirred at 20° C. for 2 hr. Then NaBH₃CN (800 mg, 12.73 mmol) was added to the mixture at 20° C., the mixture was stirred at 20° C. for 14 hr. LCMS showed a peak (87%) with desired mass. To the reaction mixture was added H₂O (30 mL), then it was concentrated to remove MeOH, diluted with EtOAc (40 mL) and then saturated NaHCO₃ solution was added to the mixture until pH=8-9 at 0° C. The mixture was extracted with EtOAc (50 mL×2), then the combined organic layers were washed with brine (40 mL×2), dried over Na₂SO₄, filtered and the filtrate was concentrated under reduced pressure. The product was purified by prep-HPLC (column: Waters Xbridge C18 150×50 mm×10 μm; mobile phase: [water (NH₄HCO₃)-ACN]; B %: 54%-84%, 10 min, Column Temp: 30° C.) and the eluent was lyophilized to afford tert-butyl 4-((((1r,4r)-4-(((benzyloxy)carbonyl)amino)cyclohexyl)methyl)(methyl)amino)piperidi ne-1-carboxylate (300 mg, 652.71 μmol, 43.41% yield) as a light yellow solid. MS(M+Na)⁺=482.3.

### Step 4. Synthesis of benzyl ((1r,4r)-4-((methyl(piperidin-4-yl)amino)methyl)cyclohexyl)carbamate (5)

It was synthesized by similar method described in step 1 to obtain benzyl ((1r,4r)-4-((methyl(piperidin-4-yl)amino)methyl)cyclohexyl)carbamate (170 mg, crude, HCl salt) as a light yellow solid. MS(M+H)⁺=360.3.

### Step 5. Synthesis of benzyl ((1r,4r)-4-(((1-(6-(2,6-dioxopiperidin-3-yl)pyridin-3-yl)piperidin-4-yl)(methyl)amino)methyl)cyclohexyl)carbamate (7)

It was synthesized by similar method described in step 3 of Example 8 to obtain benzyl ((1r,4r)-4-(((1-(6-(2,6-dioxopiperidin-3-yl)pyridin-3-yl)piperidin-4-yl)(methyl)amino) methyl)cyclohexyl)carbamate (90 mg, 164.33 mol, 36.85% yield) as a light yellow solid. MS(M+H)⁺=548.3.

### Step 6. Synthesis of 3-(5-(4-((((1r,4r)-4-aminocyclohexyl)methyl)(methyl)amino)piperidin-1-yl)pyridin-2-yl)piperidine-2,6-dione (8)

It was synthesized by similar method described in step 2 of Example 4 to obtain 3-(5-(4-((((1r,4r)-4-aminocyclohexyl)methyl)(methyl)amino)piperidin-1-yl)pyridin-2-yl)piperidine-2,6-dione (77 mg, crude, TFA salt) as a light yellow oil. MS(M+H)⁺=414.3.

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)pyridin-3-yl)piperidin-4-yl)(methyl)amino)methyl)cyclohexyl)-3-methoxybenzamide (Compound 9)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)pyridin-3-yl)piperidin-4-yl)(methyl)amino)methyl)cyclohexyl)-3-methoxybenzamide (26.3 mg, 23.35 mol, 13.06% yield, 94.7% purity, 2TFA salt) as a white solid. MS(M+H)⁺=843.5.

¹H NMR (400 MHz, CD₃OD) δ=8.36 (d, J=2.8 Hz, 1H), 8.21 (d, J=8.6 Hz, 1H), 8.18-8.14 (m, 1H), 7.86 (dd, J=2.6, 9.2 Hz, 1H), 7.64-7.56 (m, 2H), 7.55-7.50 (m, 1H), 5.11-4.99 (m, 2H), 4.17-4.07 (m, 4H), 4.02 (s, 3H), 3.96-3.87 (m, 1H), 3.63-3.53 (m, 1H), 3.41 (s, 3H), 3.28-3.21 (m, 1H), 3.10-3.00 (m, 3H), 2.93 (s, 3H), 2.81-2.74 (m, 2H), 2.45-2.35 (m, 1H), 2.33-2.16 (m, 3H), 2.13-2.02 (m, 5H), 2.00-1.82 (m, 6H), 1.75-1.66 (m, 4H), 1.59-1.47 (m, 2H), 1.38-1.26 (m, 2H).

### Example 10. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)pyridin-3-yl)piperidin-4-yl)methyl)(methyl)amino)cyclohexyl)-3-methoxybenzamide (Compound 10)

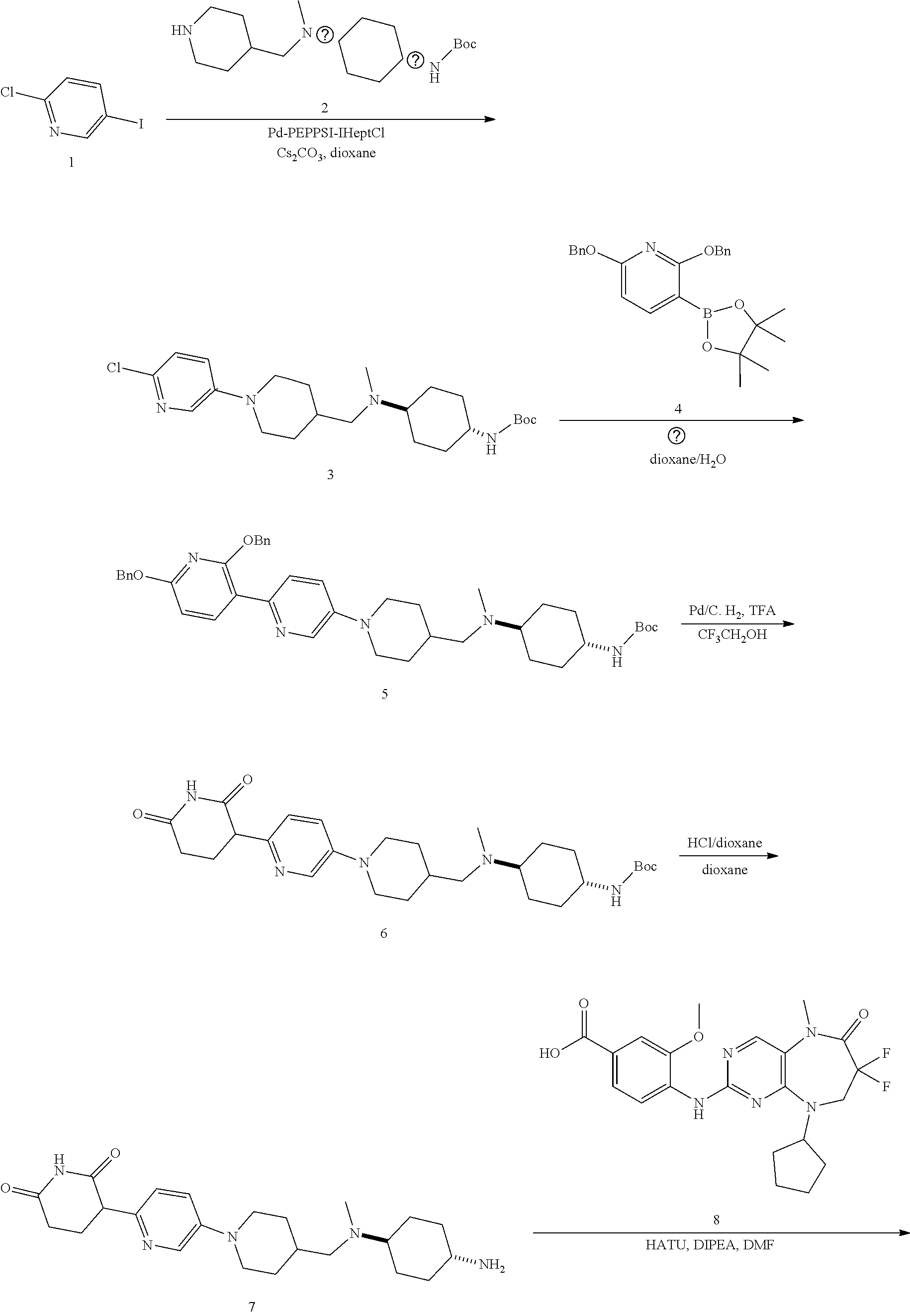

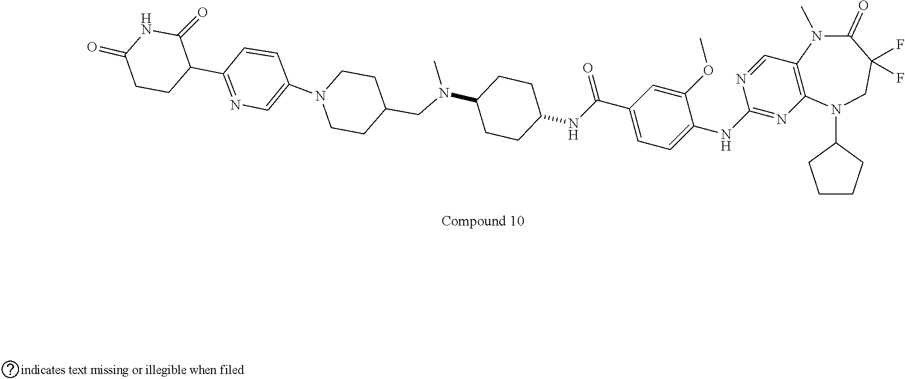

### Step 1. Synthesis of tert-butyl ((1r,4r)-4-(((1-(6-chloropyridin-3-yl)piperidin-4-yl)methyl)(methyl)amino)cyclohexyl)carbamate (3)

It was synthesized by similar method described in step 3 of Example 8 to obtain tert-butyl ((1r,4r)-4-(((1-(6-chloropyridin-3-yl)piperidin-4-yl)methyl)(methyl)amino)cyclohexyl) carbamate (550 mg, 1.26 mmol, 60.27% yield) as a light yellow solid. MS(M+H)⁺=437.2.

### Step 2. Synthesis of tert-butyl ((1r,4r)-4-(((1-(2′,6′-bis(benzyloxy)-[2,3′-bipyridin]-5-yl)piperidin-4-yl)methyl)(methyl)amino)cyclohexyl)carbamate (5)

A mixture of tert-butyl ((1r,4r)-4-(((1-(6-chloropyridin-3-yl)piperidin-4-yl)methyl)(methyl)amino)cyclohexyl) carbamate (400 mg, 915.30 μmol), 2,6-bis(benzyloxy)-3-(4,4,5,5-tetramethyl-1,3,2-dioxaborolan-2-yl)pyridine (666.67 mg, 1.60 mmol), K₃PO₄ (666.67 mg, 3.14 mmol) and CataCXium A Pd G2 (80 mg, 119.65 μmol) in dioxane (20 mL) and H₂O (5 mL) was degassed and purged with N₂ for 3 times. The resulting mixture was stirred at 80° C. for 16 h under N₂ atmosphere. LCMS showed tert-butyl ((1r,4r)-4-(((1-(6-chloropyridin-3-yl)piperidin-4-yl)methyl)(methyl)amino)cyclohexyl) carbamate was consumed completely and peak (41%) with desired mass. The reaction mixture was diluted with H₂O (15 mL), extracted with EtOAc (30 mL×2). The combined organic layers were washed with brine 90 mL (30 mL×3), dried over Na₂ SO₄, filtered. The filtrate was concentrated under reduced pressure. The residue was purified by flash silica gel chromatography (Biotage; 10 g SepaFlash® Silica Flash Column, Eluent of 2-10% DCM:MeOH gradient, 60 mL/min) to afford tert-butyl ((1r,4r)-4-(((1-(2′,6′-bis(benzyloxy)-[2,3′-bipyridin]-5-yl)piperidin-4-yl)methyl)(methyl)amino)cyclohexyl)carbamate (520 mg, 751.55 μmol, 82.11% yield) as a yellow solid. MS(M+H)⁺=692.5.

### Step 3. Synthesis of tert-butyl ((1r,4r)-4-(((1-(6-(2,6-dioxopiperidin-3-yl)pyridin-3-yl)piperidin-4-yl)methyl)(methyl)amino)cyclohexyl)carbamate (6)

It was synthesized by similar method described in step 4 of Example 1 to obtain tert-butyl ((1r,4r)-4-(((1-(6-(2,6-dioxopiperidin-3-yl)pyridin-3-yl)piperidin-4-yl)methyl)(methyl) amino)cyclohexyl)carbamate (230 mg, 447.76 mol, 96.81% yield) as a yellow oil. MS(M+H)⁺=514.3.

### Step 4. Synthesis of 3-(5-(4-((((1r,4r)-4-aminocyclohexyl)(methyl)amino)methyl)piperidin-1-yl)pyridin-2-yl)piperidine-2,6-dione (7)

It was synthesized by similar method described in step 5 of Example 1 to obtain 3-(5-(4-((((1r,4r)-4-aminocyclohexyl)(methyl)amino)methyl)piperidin-1-yl)pyridin-2-yl)piperidine-2,6-dione (156 mg, crude, HCl salt) as a light yellow solid. MS(M+H)⁺=414.3.

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)pyridin-3-yl)piperidin-4-yl)methyl)(methyl)amino)cyclohexyl)-3-methoxybenzamide (Compound 10)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)pyridin-3-yl)piperidin-4-yl)methyl)(methyl)amino)cyclohexyl)-3-methoxybenzamide (42.0 mg, 44.82 μmol, 13.37% yield, 89.95% purity) as a white solid. MS(M+H)⁺=843.5.

¹H NMR (400 MHz, DMSO-d₆) δ=10.77 (s, 1H), 8.31-8.23 (m, 2H), 8.22-8.16 (m, 1H), 8.06 (d, J=7.8 Hz, 1H), 7.95 (s, 1H), 7.53-7.44 (m, 2H), 7.35-7.27 (m, 1H), 7.15 (d, J=8.7 Hz, 1H), 4.82-4.70 (m, 1H), 4.09-3.99 (m, 2H), 3.93 (s, 3H), 3.91-3.84 (m, 1H), 3.79-3.63 (m, 3H), 2.74-2.63 (m, 2H), 2.60-2.51 (m, 2H), 2.50-2.48 (m, 3H), 2.36-2.29 (m, 1H), 2.28-2.23 (m, 2H), 2.20 (s, 3H), 2.18-2.04 (m, 2H), 1.98-1.86 (m, 4H), 1.82-1.67 (m, 6H), 1.64-1.52 (m, 5H), 1.43-1.29 (m, 4H), 1.23-1.11 (m, 2H).

### Example 11. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)pyridin-3-yl)piperidin-4-yl)methyl)(methyl)amino)cyclohexyl)-3-methoxybenzamide (Compound 11)

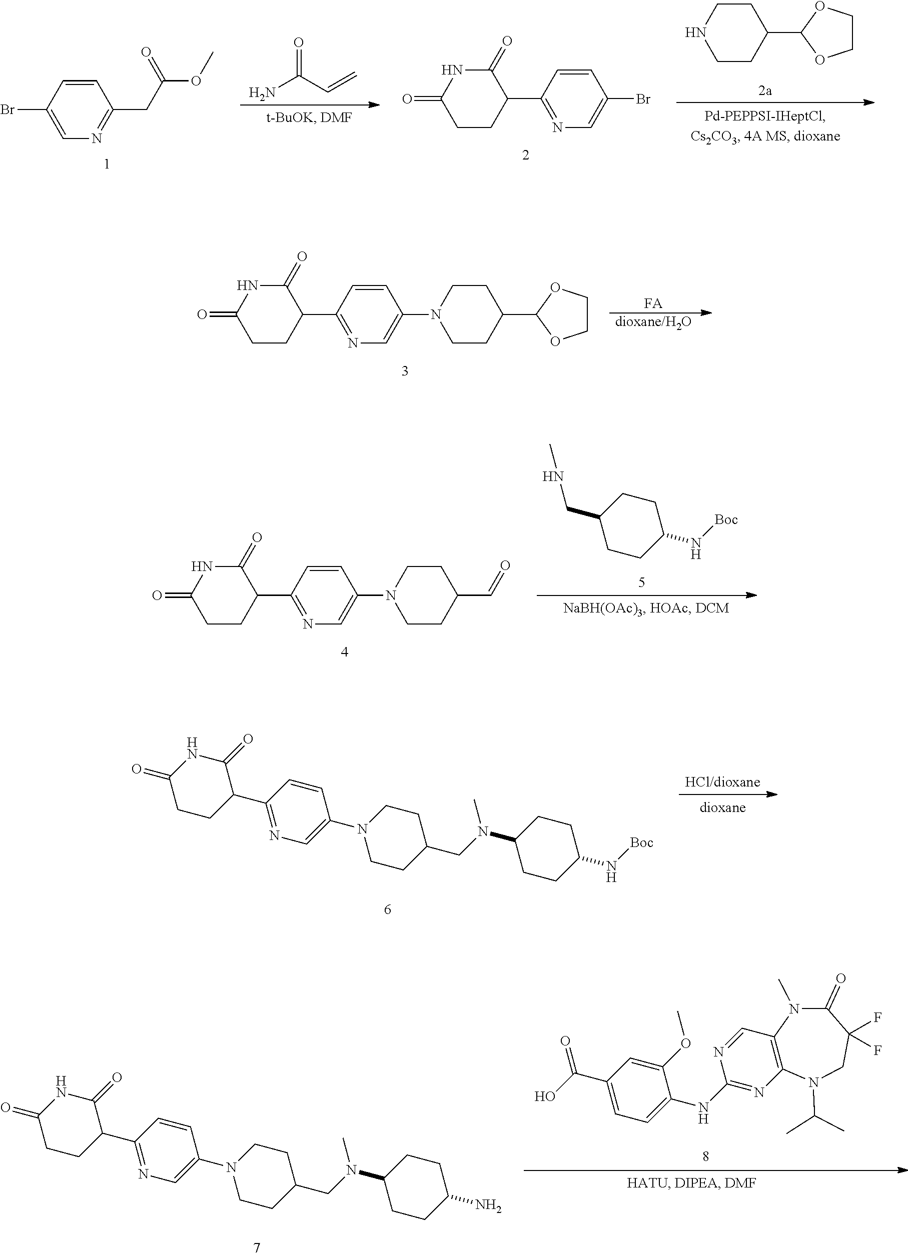

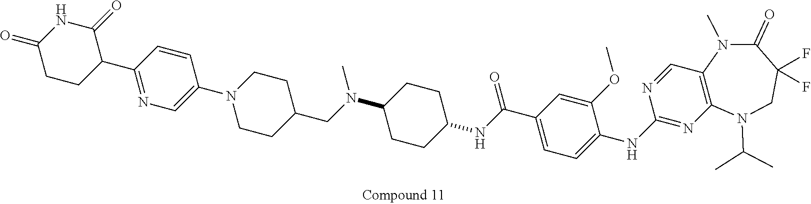

### Step 1. Synthesis of 3-(5-bromopyridin-2-yl)piperidine-2,6-dione (2)

To a solution of methyl 2-(5-bromopyridin-2-yl)acetate (2.0 g, 8.69 mmol) in DMF (15 mL) were added acrylamide (617.91 mg, 8.69 mmol, 599.91 μL) and t-BuOK (1 M, 8.69 mL) at 0° C. The mixture was stirred at 0° C. for 2 hr. TLC (Petroleum ether:EtOAc=1:1) indicated the starting material was consumed completely and one new spot formed. The reaction mixture was filtered and the filter cake was dried to give the crude product which was triturated with EtOAc:water=1:1 at 20° C. for 30 min to afford 3-(5-bromopyridin-2-yl)piperidine-2,6-dione (1.5 g, 5.57 mmol, 64.12% yield) as a white solid. MS(M+H)⁺=270.1.

¹H NMR (400 MHz, DMSO-d₆) δ=11.01-10.73 (m, 1H), 8.64 (d, J=2.0 Hz, 1H), 8.04 (dd, J=2.4, 8.3 Hz, 1H), 7.38 (d, J=8.3 Hz, 1H), 4.12-3.97 (m, 1H), 2.68-2.58 (m, 1H), 2.56-2.52 (m, 1H), 2.31-2.19 (m, 1H), 2.16-2.06 (m, 1H).

### Step 2. Synthesis of 3-(5-(4-(1,3-dioxolan-2-yl)piperidin-1-yl)pyridin-2-yl)piperidine-2,6-dione (3)

It was synthesized by similar method described in step 3 of Example 8 to obtain 3-(5-(4-(1,3-dioxolan-2-yl)-1-piperidyl)-2-pyridyl)piperidine-2,6-dione (1.1 g, 3.06 mmol, 54.85% yield, 96% purity) as a yellow solid. MS(M+H)⁺=346.2

¹H NMR (400 MHz, DMSO-d₆) δ=10.77 (s, 1H), 8.19 (d, J=2.8 Hz, 1H), 7.31 (dd, J=2.9, 8.7 Hz, 1H), 7.16 (d, J=8.7 Hz, 1H), 4.62 (d, J=4.9 Hz, 1H), 3.91-3.84 (m, 3H), 3.81-3.73 (m, 4H), 2.74-2.63 (m, 2H), 2.59-2.53 (m, 2H), 2.25-2.05 (m, 2H), 1.74 (d, J=13.8 Hz, 2H), 1.69-1.60 (m, 1H), 1.46-1.33 (m, 2H).

### Step 3. Synthesis of 1-(6-(2,6-dioxopiperidin-3-yl)pyridin-3-yl)piperidine-4-carbaldehyde (4)

To a solution of 3-(5-(4-(1,3-dioxolan-2-yl)piperidin-1-yl)pyridin-2-yl)piperidine-2,6-dione (600 mg, 1.74 mmol) in dioxane (2 mL) and H₂O (2 mL) was added FA (83.46 mg, 1.74 mmol, 6 mL). The mixture was stirred at 20° C. for 48 hours. LCMS showed 6% of the starting material remained, several new peaks were shown on LCMS and a peak (93%) with desired mass. The reaction mixture was diluted with aqueous sat. Na₂CO₃ (50 mL) at 0° C. to pH=7, and then extracted with EtOAc (100 mL×2). The combined organic layers were washed with brine (10 mL), dried over Na₂SO₄, filtered and concentrated under reduced pressure to afford 1-(6-(2,6-dioxopiperidin-3-yl)pyridin-3-yl)piperidine-4-carbaldehyde (520 mg, 1.55 mmol, 89.40% yield, 90% purity) as a yellow solid. MS(M+H+H₂O)⁺=320.2

### Step 4. Synthesis of tert-butyl ((1r,4r)-4-(((1-(6-(2,6-dioxopiperidin-3-yl)pyridin-3-yl)piperidin-4-yl)methyl)(methyl)amino)cyclohexyl)carbamate (6)

It was synthesized by similar method described in step 2 of Example 6 to obtain tert-butyl ((1r,4r)-4-(((1-(6-(2,6-dioxopiperidin-3-yl)pyridin-3-yl)piperidin-4-yl)methyl)(methyl) amino)cyclohexyl)carbamate (200 mg, 346.62 mol, 20.09% yield, 97% purity, FA) as a yellow solid. MS (M+H)⁺=514.4.

### Step 5. Synthesis of 3-(5-(4-((((1r,4r)-4-aminocyclohexyl)(methyl)amino)methyl)piperidin-1-yl)pyridin-2-yl)piperidine-2,6-dione (7)

It was synthesized by similar method described in step 5 of Example 1 to obtain 3-(5-(4-((((1r,4r)-4-aminocyclohexyl)(methyl)amino)methyl)piperidin-1-yl)pyridin-2-yl)piperidine-2,6-dione (110 mg, crude, HCl salt) as a white solid. MS(M+H)⁺=414.2

### Step 6. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)pyridin-3-yl)piperidin-4-yl)methyl)(methyl)amino)cyclohexyl)-3-methoxybenzamide (Compound 11)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)pyridin-3-yl)piperidin-4-yl)methyl)(methyl)amino)cyclohexyl)-3-methoxybenzamide (82.1 mg, 73.77 mol, 30.18% yield, 93.9% purity, 2TFA salt) as a white solid. MS(M+H)⁺=817.4.

¹H NMR (400 MHz, DMSO-d₆) δ=10.94 (s, 1H), 8.84 (dd, J=3.9, 8.9 Hz, 1H), 8.31 (d, J=2.3 Hz, 1H), 8.27-8.13 (m, 4H), 7.74-7.61 (m, 1H), 7.56-7.38 (m, 3H), 4.95-4.82 (m, 1H), 4.14-4.02 (m, 3H), 3.93 (s, 3H), 3.90-3.84 (m, 2H), 3.32 (s, 3H), 3.23-3.19 (m, 1H), 2.99-2.88 (m, 2H), 2.87-2.77 (m, 5H), 2.65-2.57 (m, 2H), 2.45-2.39 (m, 1H), 2.31-2.20 (m, 1H), 2.15-2.06 (m, 2H), 2.04-1.93 (m, 5H), 1.85-1.75 (m, 1H), 1.74-1.59 (m, 2H), 1.52-1.40 (m, 2H), 1.39-1.28 (m, 2H), 1.24 (d, J=6.7 Hz, 6H).

### Example 12. Synthesis of 4-((7,7-difluoro-5,9-dimethyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-methylpyridin-3-yl)piperidin-4-yl)methyl)(methyl)amino)cyclohexyl)-3-methoxybenzamide (Compound 12)

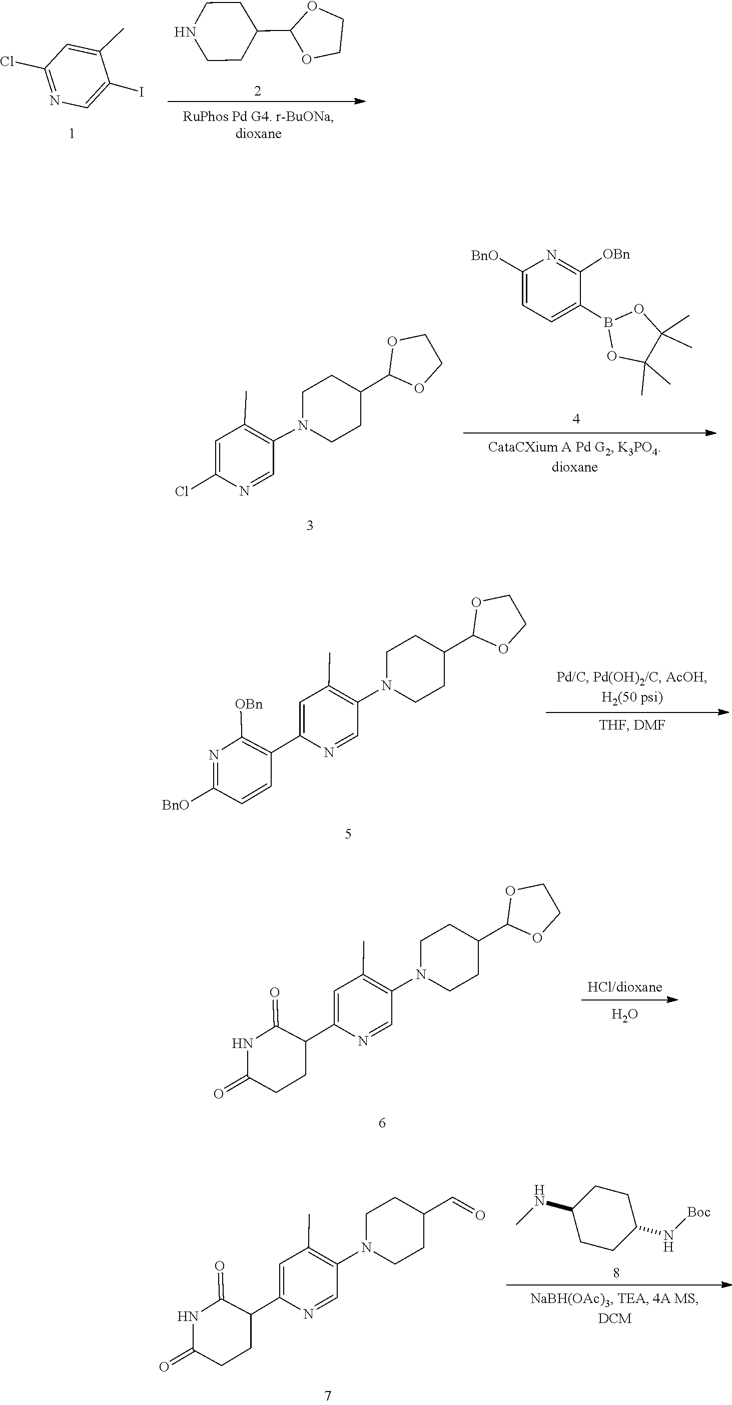

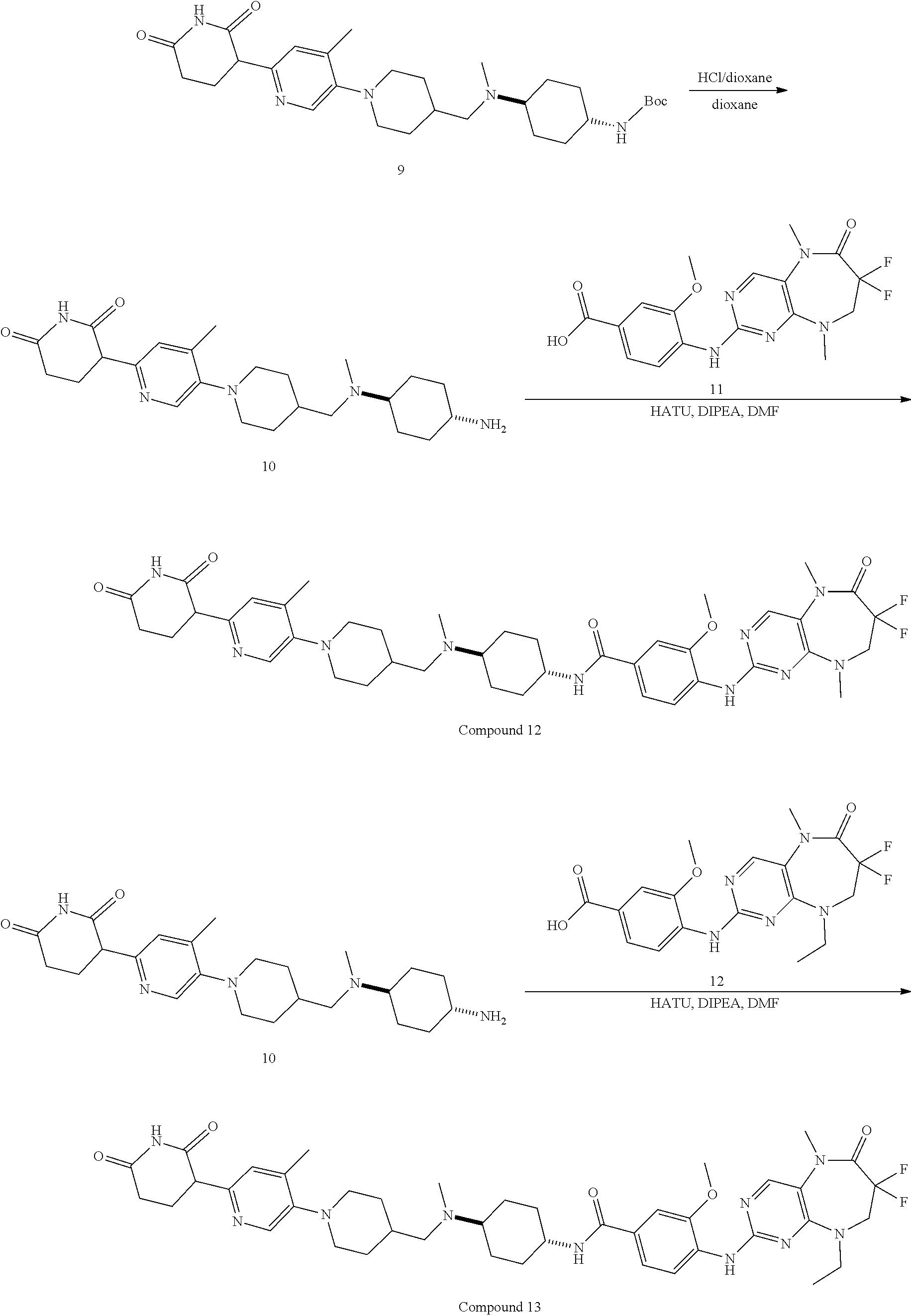

### Step 1. Synthesis of 5-(4-(1,3-dioxolan-2-yl)piperidin-1-yl)-2-chloro-4-methylpyridine (3)

A mixture of 2-chloro-5-iodo-4-methylpyridine (6.1 g, 24.07 mmol), 4-(1,3-dioxolan-2-yl)piperidine (3.78 g, 24.07 mmol), t-BuONa (6.94 g, 72.20 mmol) and RuPhos Pd G4 (409.31 mg, 481.32 μmol) in dioxane (70 mL) was stirred at 100° C. for 16 hours under N₂ atmosphere. LCMS showed the starting material was consumed completely and a peak (60%) with desired mass. The mixture was filtered and the filter cake was washed with THF (200 mL). The filtrate was concentrated in vacuum. The residue was purified by flash silica gel chromatography (40 g SepaFlash® Silica Flash Column, Eluent of 5-10% EtOAc/Petroleum ether gradient @ 200 mL/min) to afford 5-(4-(1,3-dioxolan-2-yl)piperidin-1-yl)-2-chloro-4-methylpyridine (4.35 g, 15.38 mmol, 63.92% yield) as a yellow solid. MS(M+H)⁺=283.1

### Step 2. Synthesis of 5-(4-(1,3-dioxolan-2-yl)piperidin-1-yl)-2′,6′-bis(benzyloxy)-4-methyl-2,3′-bipyridine (5)

It was synthesized by similar method described in step 2 of Example 10 to obtain 5-(4-(1,3-dioxolan-2-yl)piperidin-1-yl)-2′,6′-bis(benzyloxy)-4-methyl-2,3′-bipyridine (6 g, 11.16 mmol, 72.54% yield) as a yellow solid. MS(M+H)⁺=538.3.

### Step 3. Synthesis of 3-(5-(4-(1,3-dioxolan-2-yl)piperidin-1-yl)-4-methylpyridin-2-yl)piperidine-2,6-dione (6)

To a solution of 5-(4-(1,3-dioxolan-2-yl)piperidin-1-yl)-2′,6′-bis(benzyloxy)-4-methyl-2,3′-bipyridine (5 g, 9.30 mmol) in THF (200 mL) and DMF (40 mL) were added AcOH (20.98 g, 349.38 mmol, 20 mL), Pd/C (3 g, 2.82 mmol, 10% purity) and Pd(OH)₂/C (3 g, 4.27 mmol, 20% purity) at 30° C. The mixture was stirred at 30° C. under H₂ (50 Psi) for 70 h. LCMS showed a peak (81%) with desired mass. The mixture was filtered through a pad of celite. The filtrate was concentrated in vacuum. The mixture was diluted with water (120 mL), extracted with EtOAc (100 mL×3). The combined organic layers were washed with brine (500 mL×2), dried over Na₂SO₄, filtered. The filtrate was concentrated in vacuum. The crude product was triturated with EtOAc/Petroleum ether=1/1 (50 mL) at 20° C. for 30 min to afford 3-(5-(4-(1,3-dioxolan-2-yl)piperidin-1-yl)-4-methylpyridin-2-yl)piperidine-2,6-dione (2.13 g, 5.93 mmol, 63.72% yield) as a yellow solid. MS(M+H)⁺=360.2.

### Step 4. Synthesis of 1-(6-(2,6-dioxopiperidin-3-yl)-4-methylpyridin-3-yl)piperidine-4-carbaldehyde (7)

It was synthesized by similar method described in step 3 of Example 11, changed FA to HCl/dioxane (4 M, 20 mL) to obtain 1-(6-(2,6-dioxopiperidin-3-yl)-4-methylpyridin-3-yl)piperidine-4-carbaldehyde (240 mg, crude) as a yellow solid. MS(M+H)⁺=316.2.

### Step 5. Synthesis of tert-butyl ((1r,4r)-4-(((1-(6-(2,6-dioxopiperidin-3-yl)-4-methylpyridin-3-yl)piperidin-4-yl)methyl)(methyl)amino)cyclohexyl)carbamate (9)

It was synthesized by similar method described in step 6 of Example 1 to obtain tert-butyl ((1r,4r)-4-(((1-(6-(2,6-dioxopiperidin-3-yl)-4-methylpyridin-3-yl)piperidin-4-yl)methyl)(methyl)amino)cyclohexyl)carbamate (170 mg, 322.15 mol, 42.33% yield) as a yellow solid. MS(M+H)⁺=528.3.

### Step 6. Synthesis of 3-(5-(4-((((1r,4r)-4-aminocyclohexyl)(methyl)amino)methyl)piperidin-1-yl)-4-meth ylpyridin-2-yl)piperidine-2,6-dione (10)

It was synthesized by similar method described in step 5 of Example 1 to obtain 3-(5-(4-((((1r,4r)-4-aminocyclohexyl)(methyl)amino)methyl)piperidin-1-yl)-4-methylpyridin-2-yl)piperidine-2,6-dione (150 mg, HCl salt) as a white solid. MS(M+H)⁺=428.3.

### Step 7. Synthesis of 4-((7,7-difluoro-5,9-dimethyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-methylpyridin-3-yl)piperidin-4-yl)methyl)(methyl)amino)cyclohexyl)-3-methoxybenzamide (Compound 12)

It was synthesized by similar method described in step 10 of Example 2 to obtain 4-((7,7-difluoro-5,9-dimethyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-methylpyridin-3-yl)piperidin-4-yl)methyl)(methyl)amino)cyclohexyl)-3-methoxybenzamide (36.9 mg, 44.12 mol, 27.30% yield, 96% purity) as a white solid. MS(M+H)⁺=803.3.

¹H NMR (400 MHz, DMSO-d₆) δ=10.83-10.76 (m, 1H), 8.34 (d, J=8.3 Hz, 1H), 8.29 (s, 1H), 8.13 (s, 1H), 8.07 (d, J=7.8 Hz, 1H), 7.96 (s, 1H), 7.53-7.47 (m, 2H), 7.13 (s, 1H), 4.17 (t, J=14.4 Hz, 2H), 3.94 (s, 3H), 3.91-3.85 (m, 1H), 3.77-3.68 (m, 1H), 3.31 (s, 3H), 3.13-3.08 (m, 2H), 3.06 (s, 3H), 2.64-2.57 (m, 2H), 2.45-2.40 (m, 2H), 2.31-2.65 (m, 3H), 2.24 (s, 3H), 2.21 (s, 3H), 2.14-2.07 (m, 2H), 1.92-1.87 (m, 2H), 1.83-1.75 (m, 4H), 1.55-1.49 (m, 1H), 1.39-1.31 (m, 4H), 1.27-1.20 (m, 2H).

### Example 13. Synthesis of N((1r,4r)-4-(((1-(6-(2,6-dioxopiperidin-3-yl)-4-methylpyridin-3-yl)piperidin-4-yl)methyl)(methyl)amino)cyclohexyl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (Compound 13)

It was synthesized by similar method described in step 10 of Example 2 to obtain N((1r,4r)-4-(((1-(6-(2,6-dioxopiperidin-3-yl)-4-methylpyridin-3-yl)piperidin-4-yl)methyl)(methyl)amino)cyclohexyl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (19.3 mg, 22.44 μmol, 13.89% yield, 95% purity) as a white solid. MS(M+H)⁺=817.3.

¹H NMR (400 MHz, DMSO-d₆) δ=10.81 (s, 1H), 8.31 (d, J=8.3 Hz, 1H), 8.24 (s, 1H), 8.14 (s, 1H), 8.07 (d, J=8.2 Hz, 1H), 7.90 (s, 1H), 7.55-7.46 (m, 2H), 7.13 (s, 1H), 4.26-4.11 (m, 2H), 3.94 (s, 3H), 3.92-3.85 (m, 1H), 3.78-3.70 (m, 1H), 3.70-3.61 (m, 2H), 3.32 (s, 3H), 3.15-3.09 (m, 2H), 2.74-2.69 (m, 1H), 2.66-2.64 (m, 1H), 2.46-2.42 (m, 2H), 2.32-2.27 (m, 3H), 2.24 (s, 3H), 2.21 (s, 3H), 2.15-2.07 (m, 2H), 1.94-1.88 (m, 2H), 1.85-1.75 (m, 4H), 1.59-1.49 (m, 1H), 1.43-1.26 (m, 5H), 1.25-1.19 (m, 4H).

### Example 14. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-meth ylpyridin-3-yl)piperidin-4-yl)methyl)(methyl)amino)cyclohexyl)-3-methoxybenzamide (Compound 14)

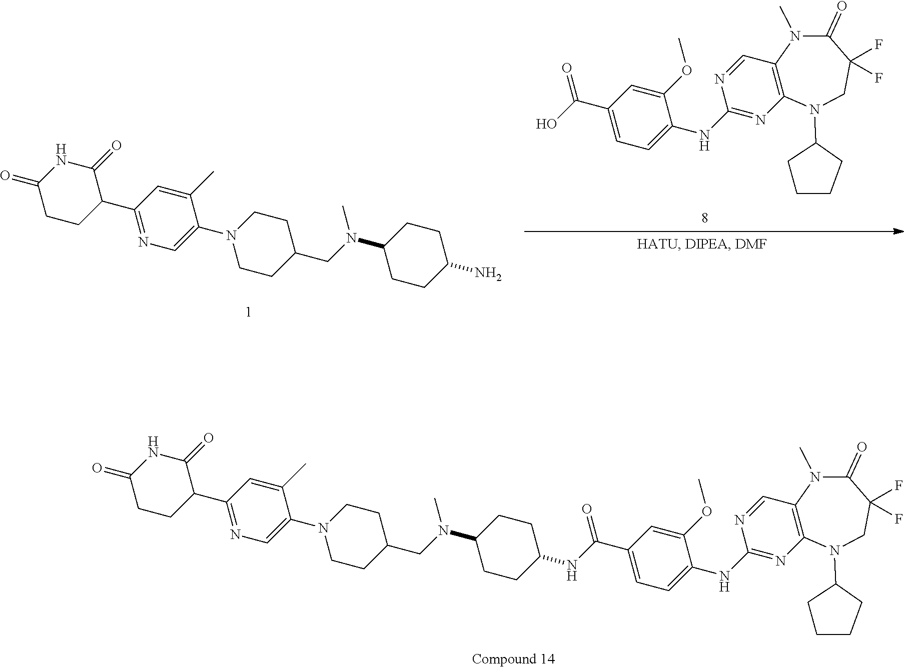

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-methylpyridin-3-yl)piperidin-4-yl)methyl)(methyl)amino)cyclohexyl)-3-methoxybenzamide (127.4 mg, 141.23 μmol, 22.57% yield, 95% purity) as a white solid. MS(M+H)⁺=857.3.

¹H NMR (400 MHz, DMSO-d₆) δ=10.79 (s, 1H), 8.32-8.18 (m, 2H), 8.17-8.12 (m, 1H), 8.07 (br dd, J=1.5, 6.1 Hz, 1H), 7.95 (s, 1H), 7.54-7.41 (m, 2H), 7.13 (s, 1H), 4.84-4.68 (m, 1H), 4.04 (br t, J=14.1 Hz, 2H), 3.93 (s, 3H), 3.88 (dd, J=5.3, 8.7 Hz, 1H), 3.75-3.70 (m, 1H), 3.36-3.55 (m, 3H), 3.17-3.07 (m, 2H), 2.67 (br dd, J=1.7, 3.5 Hz, 2H), 2.58-2.51 (m, 5H), 2.32-2.26 (m, 2H), 2.24 (s, 3H), 2.23-2.14 (m, 3H), 2.11 (br dd, J=6.4, 12.3 Hz, 1H), 1.92 (br dd, J=3.1, 10.2 Hz, 4H), 1.88-1.77 (m, 3H), 1.77-1.65 (m, 3H), 1.65-1.49 (m, 5H), 1.39 (br d, J=5.1 Hz, 4H), 1.27 (br s, 1H).

### Example 15. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-methylpyridin-3-yl)piperidin-4-yl)methyl)(methyl)amino)cyclohexyl)-3-methoxybenzamide (Compound 15)

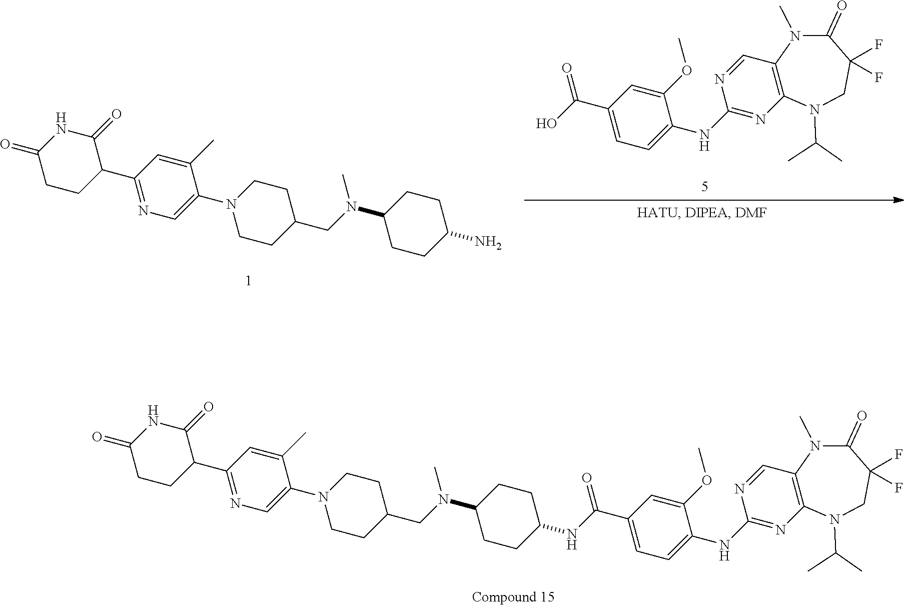

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-methylpyridin-3-yl)piperidin-4-yl)methyl)(methyl)amino)cyclohexyl)-3-methoxybenzamide (65.7 mg, 53.21 μmol, 17.25% yield, 95% purity, 3TFA salt) as a white solid. MS(M+H)⁺=831.3.

¹H NMR (400 MHz, CD₃OD) δ=8.42-8.31 (m, 1H), 8.12 (s, 1H), 8.10-7.98 (m, 1H), 7.83-7.72 (m, 1H), 7.58 (s, 1H), 7.56-7.51 (m, 1H), 5.21-5.04 (m, 1H), 4.83-4.62 (m, 1H), 4.16 (br t, J=11.8 Hz, 2H), 4.01 (s, 3H), 3.99-3.92 (m, 1H), 3.40 (s, 3H), 3.37 (br s, 3H), 3.34-3.27 (m, 1H), 3.12-3.03 (m, 1H), 2.94 (s, 5H), 2.85-2.73 (m, 2H), 2.63-2.51 (m, 3H), 2.50-2.38 (m, 1H), 2.37-2.28 (m, 1H), 2.28-2.13 (m, 4H), 2.12-2.00 (m, 2H), 1.99-1.92 (m, 1H), 1.89-1.71 (m, 2H), 1.69-1.49 (m, 4H), 1.32 (d, J=6.7 Hz, 6H).

### Example 16. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-methylpyridin-3-yl)piperidin-4-yl)(methyl)amino)methyl)cyclohexyl)-3-methoxybenzamide (Compound 16)

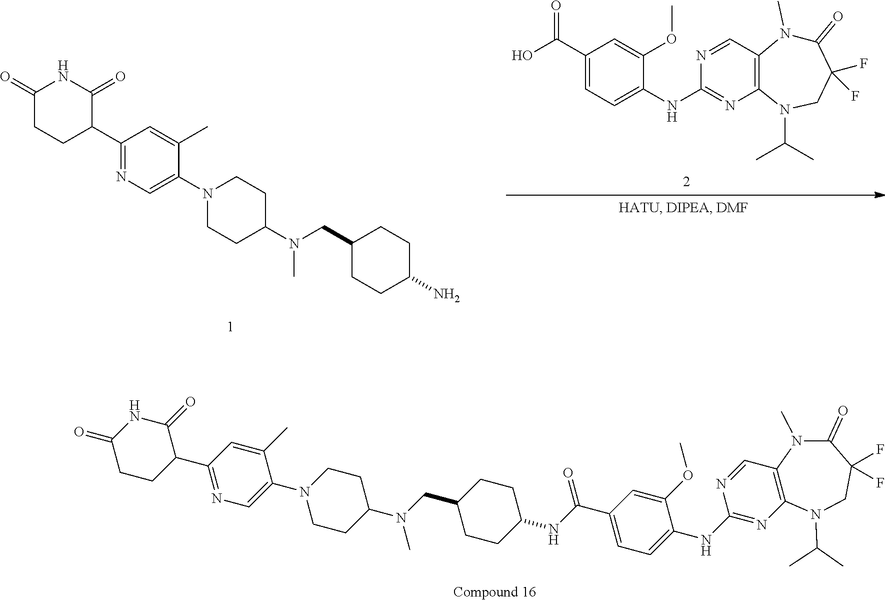

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-methylpyridin-3-yl)piperidin-4-yl)(methyl)amino)methyl)cyclohexyl)-3-methoxybenzamide (115.3 mg, 103.43 mol, 25.64% yield, 95% purity, 2TFA salt) as a white solid. MS(M+H)⁺=831.3.

¹H NMR (400 MHz, CD₃OD) δ=8.35 (br d, J=4.9 Hz, 1H), 8.14-8.05 (m, 2H), 7.75-7.65 (m, 1H), 7.58 (s, 1H), 7.54 (dd, J=1.4, 8.4 Hz, 1H), 5.15-5.08 (m, 1H), 4.84 (br d, J=1.0 Hz, 1H), 4.15 (br t, J=11.9 Hz, 2H), 4.01 (s, 3H), 3.91-3.80 (m, 1H), 3.57-3.43 (m, 3H), 3.40 (s, 3H), 3.29-3.21 (m, 1H), 3.10-2.96 (m, 3H), 2.95 (s, 3H), 2.84-2.72 (m, 2H), 2.56 (br d, J=3.3 Hz, 3H), 2.48-2.37 (m, 1H), 2.36-2.23 (m, 2H), 2.20 (br d, J=10.5 Hz, 1H), 2.15-1.99 (m, 5H), 1.98-1.83 (m, 2H), 1.62-1.45 (m, 2H), 1.32 (d, J=6.8 Hz, 8H).

### Example 17. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(6-(2,6-dioxopiperidin-3-yl)-4-methylpyridin-3-yl)piperidin-4-yl)methyl)-7-azaspiro[3.5]nonan-2-yl)-3-methoxybenzamide (Compound 17)

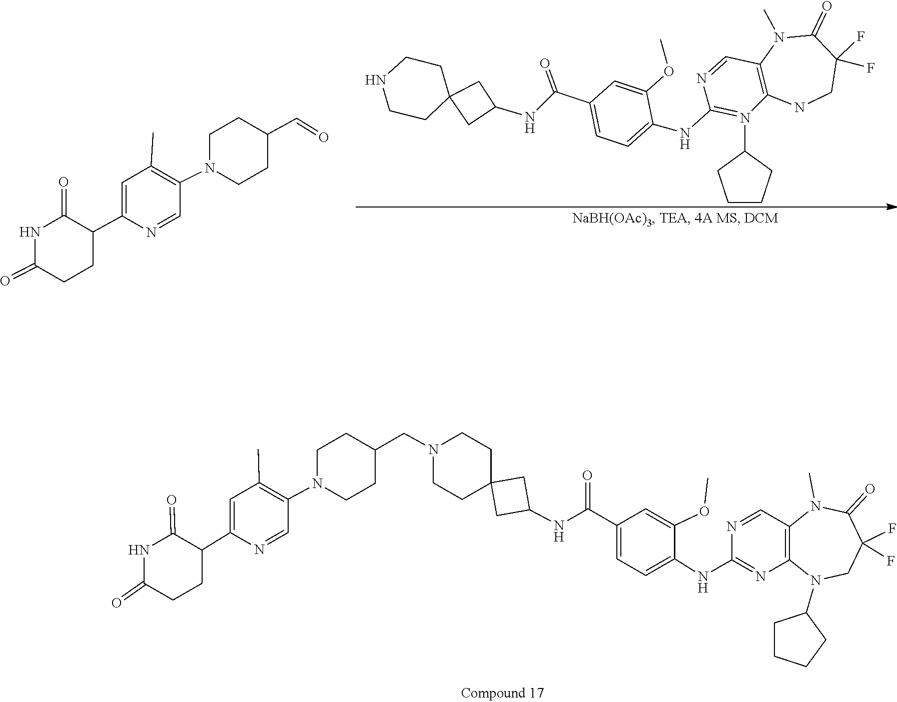

It was synthesized by similar method described in step 5 of Example 12 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(6-(2,6-dioxopiperidin-3-yl)-4-methylpyridin-3-yl)piperidin-4-yl)methyl)-7-azaspiro[3.5]nonan-2-yl)-3-methoxybenzamide (70.3 mg, 78.47 mol, 12.37% yield, 97% purity) as a white solid. MS(M+H)⁺=869.4.

¹H NMR (400 MHz, DMSO-d₆) δ=10.80 (s, 1H), 8.44 (br d, J=7.2 Hz, 1H), 8.34-8.23 (m, 2H), 8.13 (s, 1H), 7.97 (s, 1H), 7.57-7.43 (m, 2H), 7.13 (s, 1H), 4.86-4.69 (m, 1H), 4.49-4.31 (m, 1H), 4.05 (br t, J=14.1 Hz, 2H), 3.95 (s, 3H), 3.89 (dd, J=5.4, 8.7 Hz, 1H), 3.33-3.31 (m, 3H), 3.10 (br d, J=11.0 Hz, 2H), 2.68 (br t, J=11.4 Hz, 2H), 2.59-2.52 (m, 2H), 2.41-2.29 (m, 2H), 2.25-2.23 (m, 4H), 2.21-2.05 (m, 6H), 2.00-1.91 (m, 2H), 1.88-1.76 (m, 4H), 1.74-1.68 (m, 2H), 1.59 (br dd, J=4.8, 12.7 Hz, 9H), 1.34-1.18 (m, 3H).

### Example 18. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(6-(2,6-dioxopiperidin-3-yl)-2-methylpyridin-3-yl)piperidin-4-yl)methyl)-7-azaspiro[3.5]nonan-2-yl)-3-methoxybenzamide (Compound 18)

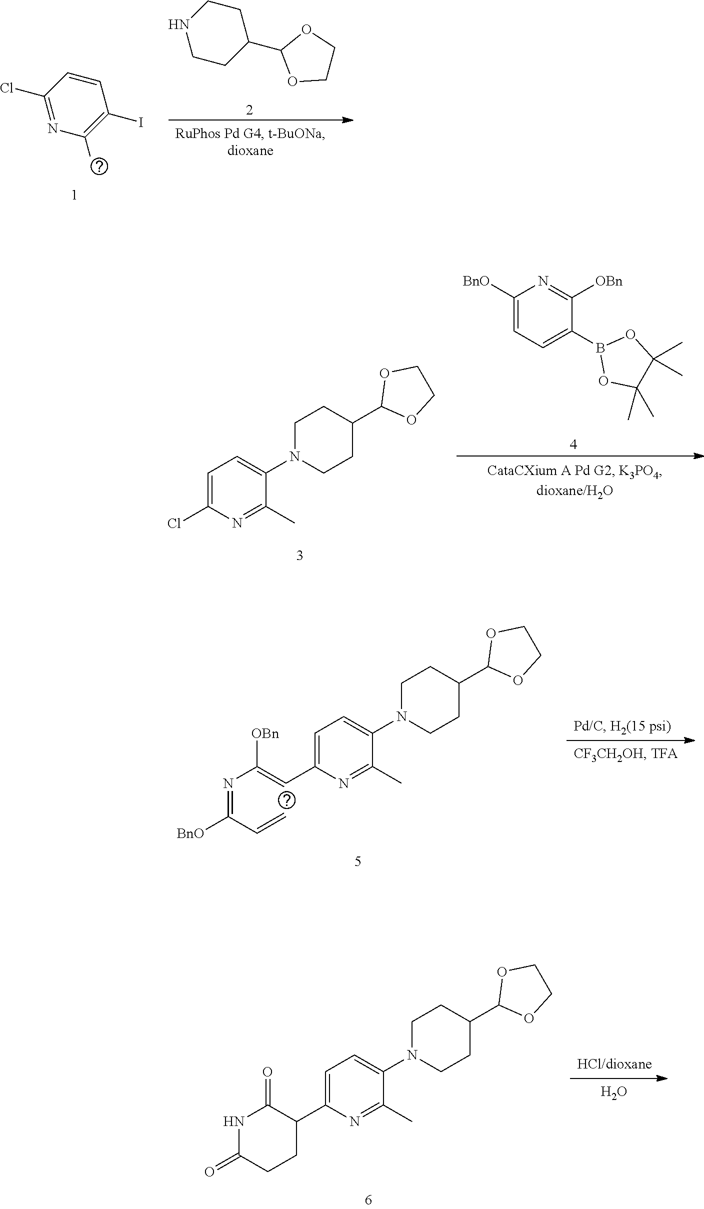

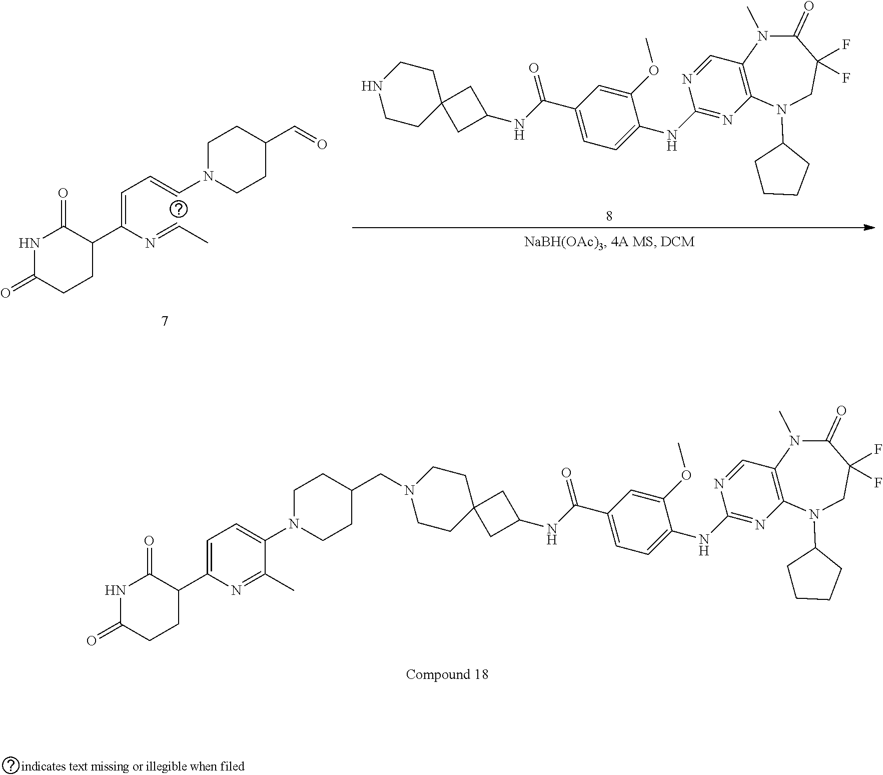 [SMILES: C/C=N/C(=C\C=C\N1CCC(C=O)CC1)C1CCC(=O)NC1=O ; COc1cc(C(=O)NC2CC3(CCNCC3)C2)ccc1Nc1ncc2c(n1)N(C1CCCC1)CC(F)(F)C(=O)N2C ; COc1cc(C(=O)NC2CC3(CCN(CC4CCN(c5ccc(C6CCC(=O)NC6=O)nc5C)CC4)CC3)C2)ccc1Nc1ncc2c(n1)N(C1CCCC1)CC(F)(F)C(=O)N2C]

### Step 1. Synthesis of 3-(4-(1,3-dioxolan-2-yl)piperidin-1-yl)-6-chloro-2-methylpyridine (3)

It was synthesized by similar method described in step 1 of Example 12 to obtain 3-(4-(1,3-dioxolan-2-yl)piperidin-1-yl)-6-chloro-2-methylpyridine (1.3 g, 4.60 mmol, 58.27% yield) as a yellow oil. MS(M+H)⁺=283.1.

### Step 2. Synthesis of 5-(4-(1,3-dioxolan-2-yl)piperidin-1-yl)-2′,6′-bis(benzyloxy)-6-methyl-2,3′-bipyridine (5)

It was synthesized by similar method described in step 2 of Example 12 to obtain 5-(4-(1,3-dioxolan-2-yl)piperidin-1-yl)-2′,6′-bis(benzyloxy)-6-methyl-2,3′-bipyridine (1.9 g, 3.53 mmol, 90.84% yield) as a light yellow solid. MS(M+H)⁺=538.3.

### Step 3. Synthesis of 3-(5-(4-(1,3-dioxolan-2-yl)piperidin-1-yl)-6-methylpyridin-2-yl)piperidine-2,6-dione (6)

It was synthesized by similar method described in step 4 of Example 1 to obtain 3-(5-(4-(1,3-dioxolan-2-yl)piperidin-1-yl)-6-methylpyridin-2-yl)piperidine-2,6-dione (1.0 g, crude) as a yellow oil. MS(M+H)⁺=360.2.

### Step 4. Synthesis of 1-(6-(2,6-dioxopiperidin-3-yl)-2-methylpyridin-3-yl)piperidine-4-carbaldehyde (7)

It was synthesized by similar method described in step 4 of Example 12 to obtain 1-(6-(2,6-dioxopiperidin-3-yl)-2-methylpyridin-3-yl)piperidine-4-carbaldehyde (450 mg, crude) as a yellow solid. MS(M+H)⁺=316.1.

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(6-(2,6-dioxopiperidin-3-yl)-2-methylpyridin-3-yl)piperidin-4-yl)methyl)-7-azaspiro[3.5]nonan-2-yl)-3-methoxybenzamide (Compound 18)

It was synthesized by similar method described in step 5 of Example 12 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(6-(2,6-dioxopiperidin-3-yl)-2-methylpyridin-3-yl)piperidin-4-yl)methyl)-7-azaspiro[3.5]nonan-2-yl)-3-methoxybenzamide (92.2 mg, 102.70 μmol, 16.19% yield, 96.8% purity) as a light yellow solid. MS(M+H)⁺=869.5.

¹H NMR (400 MHz, DMSO-d₆) δ=10.78 (s, 1H), 8.49-8.40 (m, 1H), 8.31-8.24 (m, 2H), 8.01-7.93 (m, 1H), 7.53-7.46 (m, 2H), 7.38-7.31 (m, 1H), 7.13-7.07 (m, 1H), 4.83-4.73 (m, 1H), 4.45-4.34 (m, 1H), 4.11-4.00 (m, 2H), 3.95 (s, 3H), 3.91-3.85 (m, 1H), 3.31-3.24 (m, 3H), 3.09-3.02 (m, 2H), 2.64-2.54 (m, 6H), 2.42-2.32 (m, 5H), 2.24-2.15 (m, 4H), 2.15-2.04 (m, 2H), 1.98-1.91 (m, 2H), 1.86-1.77 (m, 4H), 1.75-1.69 (m, 2H), 1.68-1.54 (m, 9H), 1.32-1.23 (m, 2H).

### Example 19. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(6-(2,6-dioxopiperidin-3-yl)-4-methylpyridin-3-yl)piperidin-4-yl)methyl)-7-azaspiro[3.5]nonan-2-yl)-3-methoxybenzamide (Compound 19)

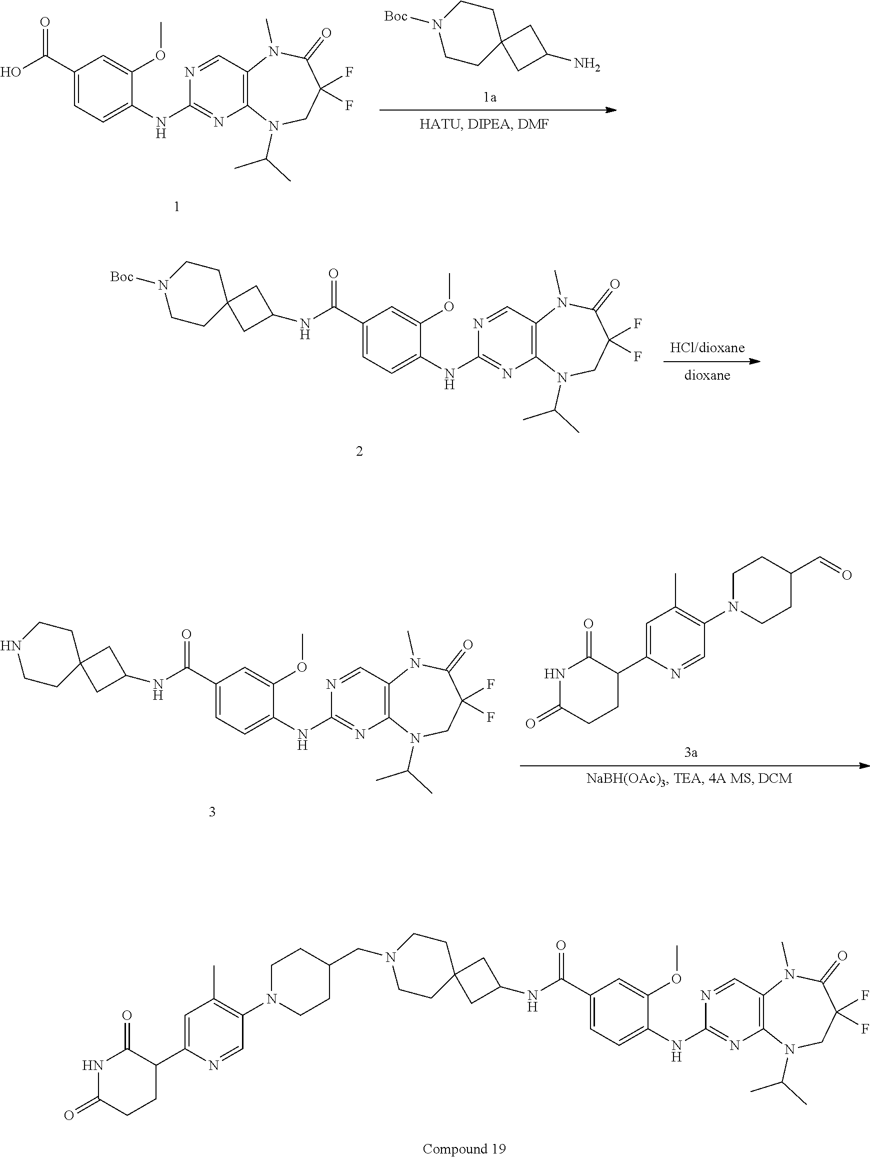

### Step 1. Synthesis of tert-butyl 2-(4-((7,7-difluoro-9-isopropyl-5-methyl-6-oxo-6,7,8,9-tetrahydro-5H-pyrimido[4, 5-b][1,4]diazepin-2-yl)amino)-3-methoxybenzamido)-7-azaspiro[3.5]nonane-7-carboxylate (2)

It was synthesized by similar method described in step 10 of Example 2 to obtain tert-butyl 2-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)-7-azaspiro[3.5]nonane-7-carboxylate (0.5 g, 706.83 mol, 59.57% yield, 91% purity) as a brown solid. MS(M+H)⁺=644.3.

### Step 2. Synthesis of 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-(7-azaspiro[3.5]nonan-2-yl)benzamide (3)

It was synthesized by similar method described in step 5 of Example 1 to obtain 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-(7-azaspiro[3.5]nonan-2-yl)benzamide (0.35 g, HCl salt) as a yellow solid, which was used for the next step directly. MS(M+H)⁺=544.3.

### Step 3. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(6-(2,6-dioxopiperidin-3-yl)-4-methylpyridin-3-yl)piperidin-4-yl)methyl)-7-azaspiro[3.5]nonan-2-yl)-3-methoxybenzamide (Compound 19)

It was synthesized by similar method described in step 5 of Example 12 to obtain 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(6-(2,6-dioxopiperidin-3-yl)-4-methylpyridin-3-yl)piperidin-4-yl)methyl)-7-azaspiro[3.5]nonan-2-yl)-3-methoxybenzamide (13.4 mg, 11.39 mol, 56.75% yield, 91% purity, 2TFA salt) as a white solid. MS(M+H)⁺=843.4

¹H NMR (400 MHz, CD₃OD) δ=8.30 (s, 1H), 8.19 (d, J=8.3 Hz, 1H), 8.12 (s, 1H), 7.67 (s, 1H), 7.57 (d, J=1.7 Hz, 1H), 7.54 (dd, J=1.9, 8.4 Hz, 1H), 5.15-5.05 (m, 1H), 4.84-4.81 (m, 1H), 4.62-4.50 (m, 1H), 4.12 (t, J=12.3 Hz, 2H), 4.01 (s, 3H), 3.59 (br d, J=13.3 Hz, 1H), 3.53 (br d, J=12.8 Hz, 1H), 3.40 (s, 3H), 3.36-3.33 (m, 2H), 3.18-3.12 (m, 2H), 3.11-3.04 (m, 1H), 3.03-2.95 (m, 1H), 2.95-2.85 (m, 2H), 2.85-2.73 (m, 2H), 2.61-2.55 (m, 1H), 2.53 (s, 3H), 2.48-2.39 (m, 1H), 2.38-2.26 (m, 2H), 2.19-2.05 (m, 3H), 2.05-1.89 (m, 6H), 1.66-1.50 (m, 2H), 1.33 (d, J=6.7 Hz, 6H).

### Example 20. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-fluoro pyridin-3-yl)piperidin-4-yl)methyl)(methyl)amino)cyclohexyl)-3-methoxybenzamide (Compound 20)

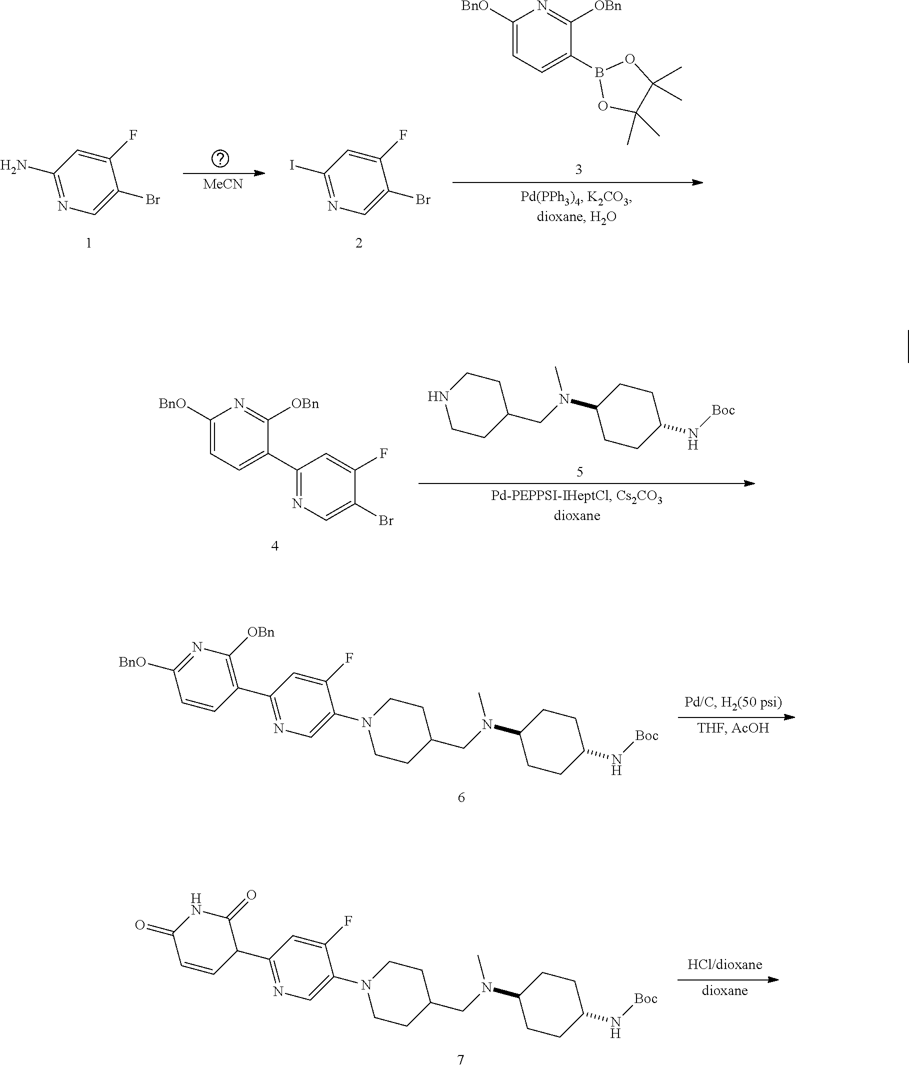

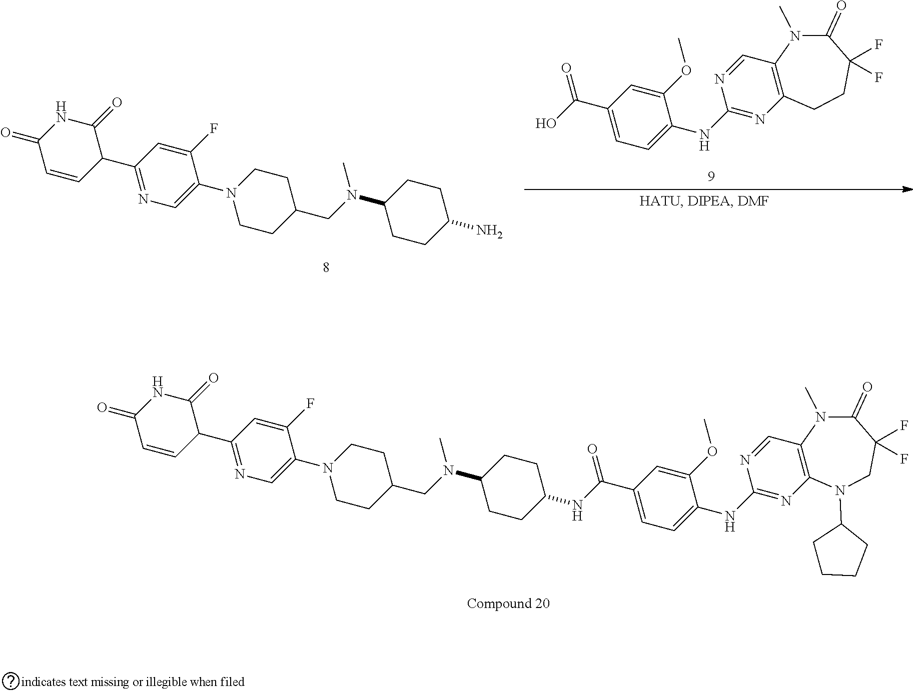

### Step 1. Synthesis of 5-bromo-4-fluoro-iodopyridin (2)

To a solution of 5-bromo-4-fluoropyridin-2-amine (1 g, 5.24 mmol) in MeCn (10 mL) were added t-BuONO (2.70 g, 26.18 mmol, 3.11 mL), CuI (9.97 g, 52.36 mmol) and I₂ (26.58 g, 104.71 mmol), the resulting mixture was stirred at 20° C. for 16 h. TLC (Petroleum ether:EtOAc 5:1) indicated the starting material was consumed completely and two major new spot with lower polarity. The reaction mixture was quenched by addition ice cold Na₂SO₃ (500 mL), extracted with EtOAc (100 mL×3). The combined organic layers were washed with brine (500 mL×2), dried over Na₂SO₄, filtered and the filtrate was concentrated in vacuum. The residue was purified by flash silica gel chromatography (Biotage; 12 g SepaFlash® Silica Flash Column, Eluent of Petroleum ether gradient @ 100 mL/min) to afford 5-bromo-4-fluoro-2-iodopyridine (397 mg, crude) as a yellow oil. MS(M+H)⁺=302.9.

### Step 2. Synthesis of 2′,6′-bis(benzyloxy)-5-bromo-4-fluoro-2,3′-bipyridine (4)

It was synthesized by similar method described in step 1 of Example 1 to obtain 2′,6′-bis(benzyloxy)-5-bromo-4-fluoro-2,3′-bipyridine (400 mg, 747.88 μmol, 60.53% yield) as a white solid. MS(M+H)⁺=465.1.

### Step 3. Synthesis of tert-butyl ((1r,4r)-4-(((1-(2′,6′-bis(benzyloxy)-4-fluoro-[2,3′-bipyridin]-5-yl)piperidin-4-yl)methyl)(methyl)amino)cyclohexyl)carbamate (6)

It was synthesized by similar method described in step 3 of Example 8 to obtain tert-butyl ((1r,4r)-4-(((1-(2′,6′-bis(benzyloxy)-4-fluoro-[2,3′-bipyridin]-5-yl)piperidin-4-yl)methy 1)(methyl)amino)cyclohexyl)carbamate (190 mg, 267.65 μmol, 41.51% yield) as a yellow solid. MS(M+H)⁺=710.5.

### Step 4. Synthesis of tert-butyl ((1r,4r)-4-(((1-(6-(2,6-dioxopiperidin-3-yl)-4-fluoropyridin-3-yl)piperidin-4-yl)methyl)(methyl)amino)cyclohexyl)carbamate (7)

A mixture of tert-butyl ((1r,4r)-4-(((1-(2′,6′-bis(benzyloxy)-4-fluoro-[2,3′-bipyridin]-5-yl)piperidin-4-yl)methy 1)(methyl)amino)cyclohexyl)carbamate (170 mg, 239.47 μmol) and Pd/C (50 mg, 46.98 μmol, 10% purity) in THF (20 mL) and AcOH (524.50 mg, 8.73 mmol, 0.5 mL) was degassed and purged with H₂ for 3 times, the resulting mixture was stirred at 30° C. for 36 h under H₂ (50 Psi) atmosphere. LCMS showed the starting material was consumed completely and a peak (50%) with desired mass. The mixture was filtered through a pad of celite. The filtrate was concentrated in vacuum. The residue was purified by prep-TLC (SiO₂, DCM:MeOH=8:1) to afford tert-butyl ((1r,4r)-4-(((1-(6-(2,6-dioxopiperidin-3-yl)-4-fluoropyridin-3-yl)piperidin-4-yl)methyl) (methyl)amino)cyclohexyl)carbamate (50 mg, 94.04 μmol, 39.27% yield) as a yellow solid. MS(M+H)⁺=532.3.

### Step 5. Synthesis of 3-(5-(4-((((1r,4r)-4-aminocyclohexyl)(methyl)amino)methyl)piperidin-1-yl)-4-fluoropyridin-2-yl)piperidine-2,6-dione (8)

It was synthesized by similar method described in step 5 of Example 1 to obtain 3-(5-(4-((((1r,4r)-4-aminocyclohexyl)(methyl)amino)methyl)piperidin-1-yl)-4-fluoropyridin-2-yl)piperidine-2,6-dione (35 mg, crude, HCl salt) as a white solid. MS(M+H)⁺=432.3.

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-fluoro pyridin-3-yl)piperidin-4-yl)methyl)(methyl)amino)cyclohexyl)-3-methoxybenzamide (Compound 20)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(((1-(6-(2,6-dioxopiperidin-3-yl)-4-fluoropyridin-3-yl)piperidin-4-yl)methyl)(methyl)amino)cyclohexyl)-3-methoxybenzamide (10.4 mg, 11.35 μmol, 15.18% yield, 94% purity) as a white solid. MS(M+H)⁺=861.4.

¹H NMR (400 MHz, DMSO-d₆) δ=10.84 (s, 1H), 8.30-8.17 (m, 3H), 8.06 (d, J=7.8 Hz, 1H), 7.95 (s, 1H), 7.52-7.44 (m, 2H), 7.23 (d, J=13.4 Hz, 1H), 4.82-4.71 (m, 1H), 4.10-3.99 (m, 2H), 3.98-3.91 (m, 4H), 3.80-3.67 (m, 1H), 3.45-3.37 (m, 2H), 3.32 (s, 3H), 2.80-2.69 (m, 2H), 2.65-2.53 (m, 2H), 2.48-2.41 (m, 1H), 2.39-2.23 (m, 4H), 2.20 (s, 3H), 2.15-2.07 (m, 1H), 1.99-1.88 (m, 4H), 1.83-1.70 (m, 5H), 1.62-1.54 (m, 4H), 1.44-1.30 (m, 4H), 1.29-1.17 (m, 3H).

### Example 21. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(6-(2,6-dioxopiperidin-3-yl)-4-fluoropyridin-3-yl)piperazin-1-yl)ethyl)piperidin-1-yl)-3-methoxybenzamide (Compound 21)

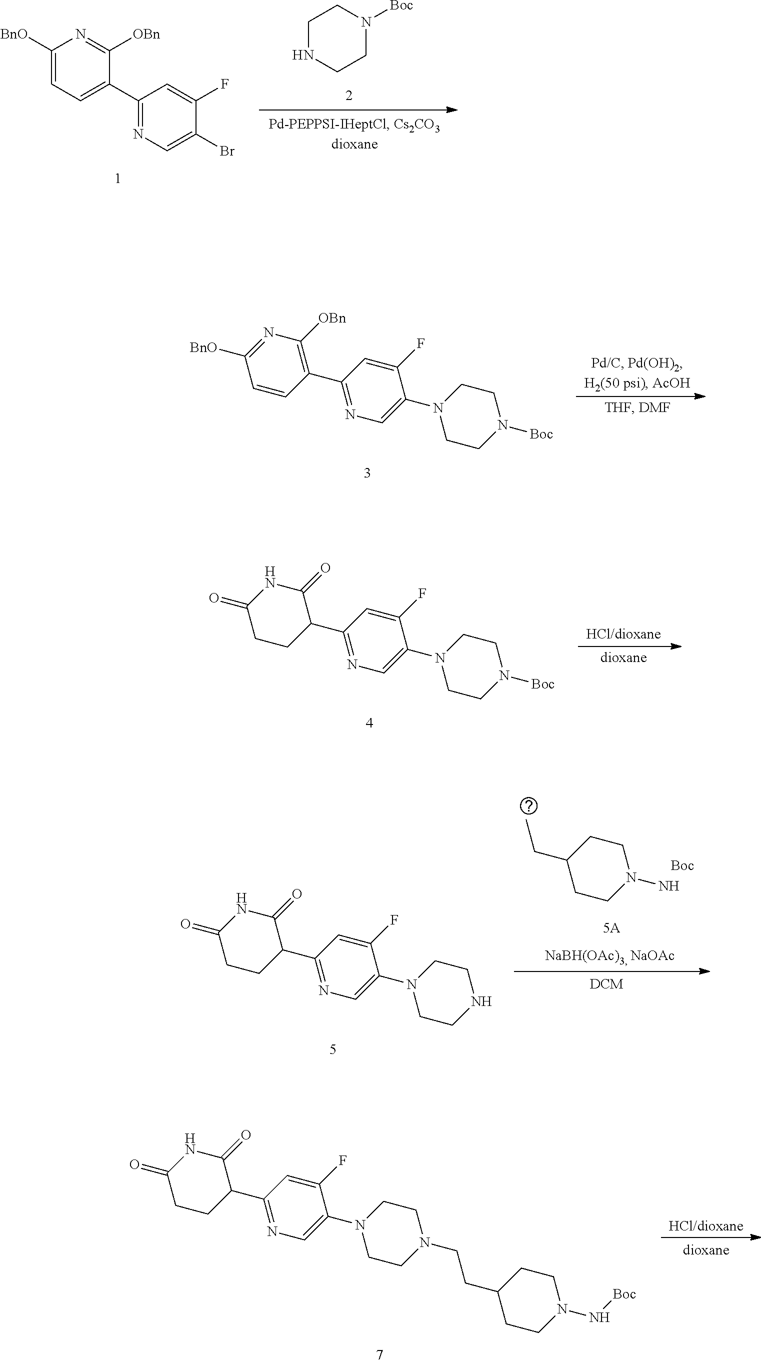

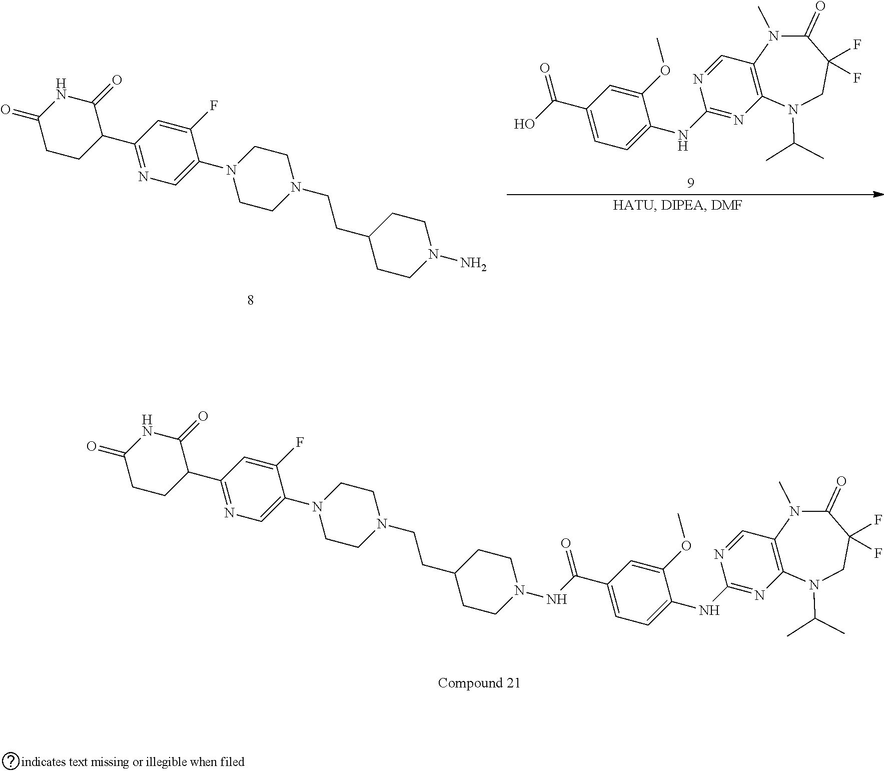 [SMILES: NN1CCC(CCN2CCN(c3cnc(C4CCC(=O)NC4=O)cc3F)CC2)CC1 ; COc1cc(C(=O)O)ccc1Nc1ncc2c(n1)N(C(C)C)CC(F)(F)C(=O)N2C ; COc1cc(C(=O)NN2CCC(CCN3CCN(c4cnc(C5CCC(=O)NC5=O)cc4F)CC3)CC2)ccc1Nc1ncc2c(n1)N(C(C)C)CC(F)(F)C(=O)N2C]

### Step 1. Synthesis of tert-butyl 4-(2′,6′-bis(benzyloxy)-4-fluoro-[2,3′-bipyridin]-5-yl)piperazine-1-carboxylate (3)

It was synthesized by similar method described in step 3 of Example 8 to obtain tert-butyl 4-(2′,6′-bis(benzyloxy)-4-fluoro-[2,3-bipyridin]-5-yl)piperazine-1-carboxylate (850 mg, 1.49 mmol, 69.31% yield) as a white solid. MS(M+H)⁺=9571.4.

### Step 2. Synthesis of tert-butyl 4-(6-(2,6-dioxopiperidin-3-yl)-4-fluoropyridin-3-yl)piperazine-1-carboxylate (4)

It was synthesized by similar method described in step 3 of Example 12 to obtain tert-butyl 4-(6-(2,6-dioxopiperidin-3-yl)-4-fluoropyridin-3-yl)piperazine-1-carboxylate (350 mg) as a white solid. MS(M+H)⁺393.4.

### Step 3. Synthesis of 3-(4-fluoro-5-(piperazin-1-yl)pyridin-2-yl)piperidine-2,6-dione (5)

It was synthesized by similar method described in step 5 of Example 1 to obtain 3-(4-fluoro-5-(piperazin-1-yl)pyridin-2-yl)piperidine-2,6-dione (300 mg, HCl salt) as a white solid which was used into the next step directly. MS(M+H)⁺=293.3.

### Step 4. Synthesis of tert-butyl (4-(2-(4-(6-(2,6-dioxopiperidin-3-yl)-4-fluoropyridin-3-yl)piperazin-1-yl)ethyl)piperidin-1-yl)carbamate (7)

It was synthesized by similar method described in step 1 of Example 4 to obtain tert-butyl (4-(2-(4-(6-(2,6-dioxopiperidin-3-yl)-4-fluoropyridin-3-yl)piperazin-1-yl)ethyl)piperidin-1-yl)carbamate (350 mg, crude) as a yellow oil. MS(M+H)⁺=519.4.

### Step 5. Synthesis of 3-(5-(4-(2-(1-aminopiperidin-4-yl)ethyl)piperazin-1-yl)-4-fluoropyridin-2-yl)piperidine-2,6-dione (8)

It was synthesized by similar method described in step 3 to obtain 3-(5-(4-(2-(1-aminopiperidin-4-yl)ethyl)piperazin-1-yl)-4-fluoropyridin-2-yl)piperidine-2,6-dione (250 mg, 549.49 mol, 94.99% yield, HCl salt) as a yellow oil. MS(M+H)⁺=419.2.

### Step 6. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(6-(2,6-dioxopiperidin-3-yl)-4-fluoropyridin-3-yl)piperazin-1-yl)ethyl)piperidin-1-yl)-3-methoxybenzamide (Compound 21)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(6-(2,6-dioxopiperidin-3-yl)-4-fluoropyridin-3-yl)piperazin-1-yl)ethyl)piperidin-1-yl)-3-methoxybenzamide (36.9 mg, 43.10 mol, 7.84% yield, 96% purity) as a white solid. MS(M+H)⁺=822.5.

¹H NMR (400 MHz, DMSO-d₆) δ=10.84 (s, 1H), 9.23 (s, 1H), 8.29 (d, J=8.3 Hz, 1H), 8.24-8.18 (m, 2H), 7.87 (s, 1H), 7.48-7.39 (m, 2H), 7.25 (d, J=13.4 Hz, 1H), 4.98-4.78 (m, 1H), 4.10-4.00 (m, 2H), 3.99-3.84 (m, 4H), 3.32-3.31 (m, 3H), 3.14-3.04 (m, 4H), 3.03-2.94 (m, 2H), 2.80-2.69 (m, 2H), 2.65-2.52 (m, 6H), 2.39-2.32 (m, 2H), 2.29-2.19 (m, 1H), 2.14-2.06 (m, 1H), 1.80-1.65 (m, 2H), 1.48-1.38 (m, 2H), 1.36-1.27 (m, 3H), 1.24 (d, J=6.8 Hz, 6H).

### Example 22. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-3-((1-(4-((2,6-dioxopiperidin-3-yl)amino) phenyl)piperidin-4-yl)methyl)-3-azabicyclo[3.1.0]hexan-6-yl)-3-methoxybenzamide (Compound 22)

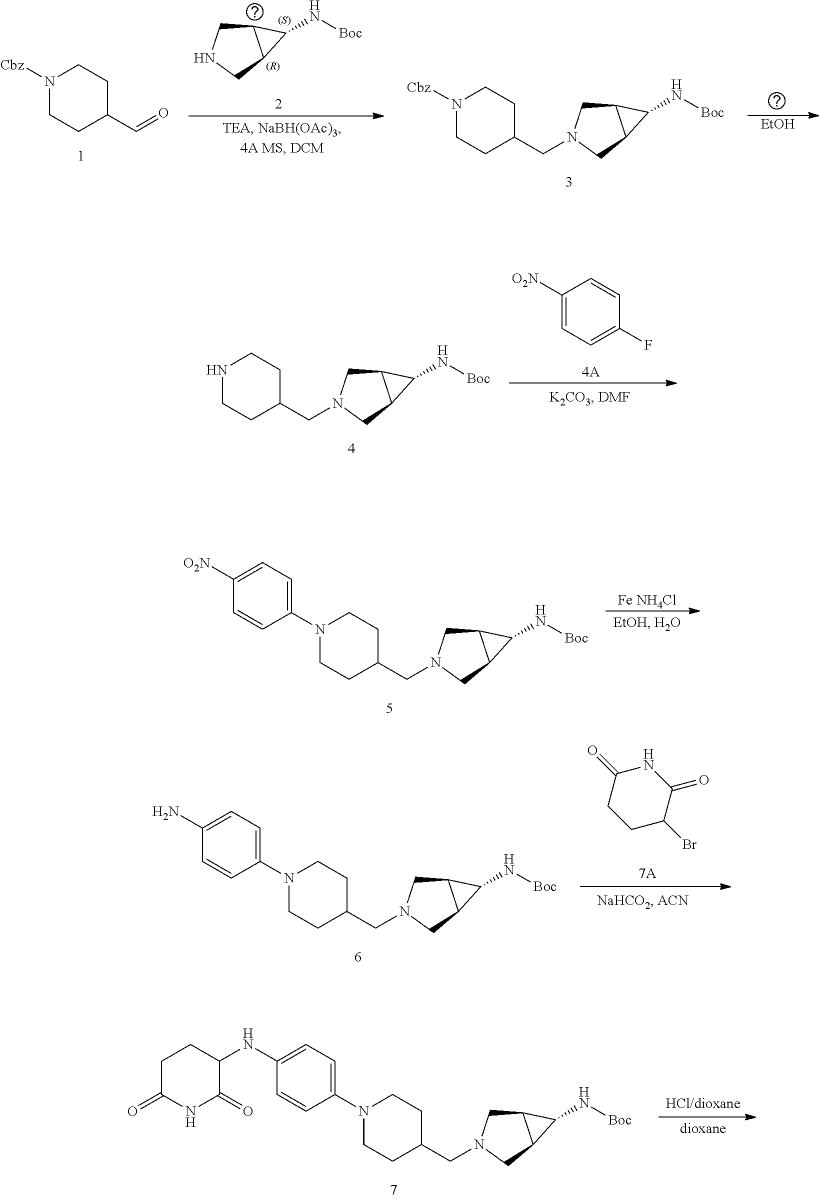

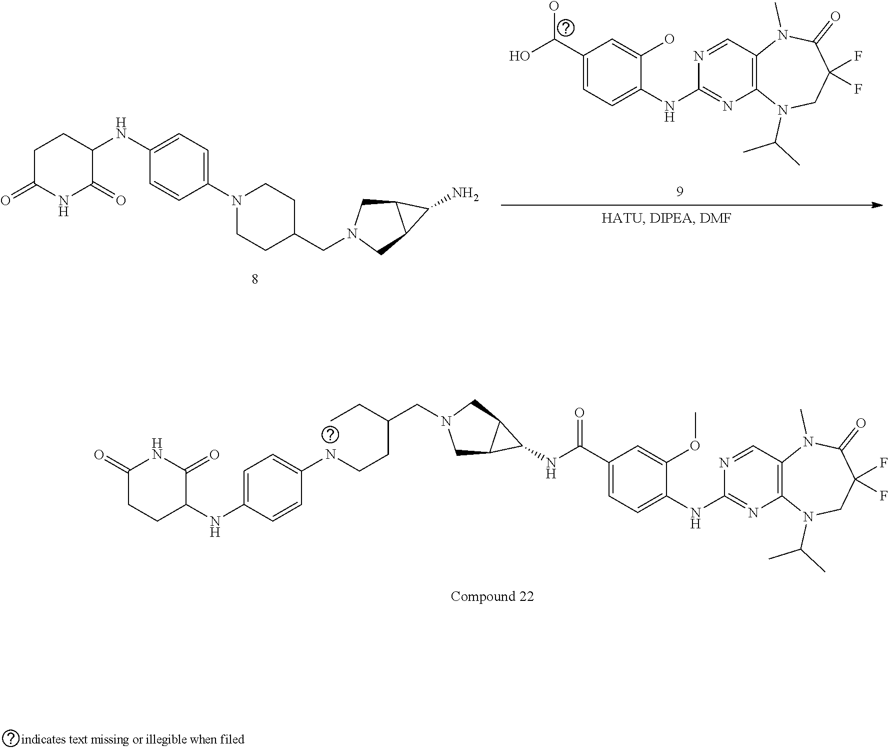 [SMILES: N[C@@H]1[C@H]2CN(CC3CCN(c4ccc(NC5CCC(=O)NC5=O)cc4)CC3)C[C@@H]12 ; CC(C)N1CC(F)(F)C(=O)N(C)c2cnc(Nc3ccc(C(O)O)cc3O)nc21 ; CCC(CCNc1ccc(NC2CCC(=O)NC2=O)cc1)CN1C[C@@H]2[C@H](C1)[C@H]2NC(=O)c1ccc(Nc2ncc3c(n2)N(C(C)C)CC(F)(F)C(=O)N3C)c(OC)c1]

### Step 1. Synthesis of benzyl 4-(((1R,5S,6s)-6-((tert-butoxycarbonyl)amino)-3-azabicyclo[3.1.0]hexan-3-yl)meth yl)piperidine-1-carboxylate (3)

To a solution of benzyl 4-formylpiperidine-1-carboxylate (1.15 g, 4.65 mmol) in DCM (20 mL) were added TEA (1.41 g, 13.95 mmol, 1.94 mL), 4A MS (50 mg) and tert-butyl ((1R,5S,6s)-3-azabicyclo[3.1.0]hexan-6-yl)carbamate (919.00 mg, 4.64 mmol) at 20° C. After stirring 10 min, NaBH(OAc)₃ (2.96 g, 13.95 mmol) was slowly added at 20° C. The mixture was stirred at 20° C. for 16 h. LCMS showed the starting material was consumed completely and 68% peak with desired mass. The reaction mixture was filtered. The filter cake was washed with MeOH (40 mL). The filtrate was concentrated in vacuum to afford crude product. The crude product was triturated with EtOAc (10 mL) at 20° C. for 2 h and filtered. The filtrate was purified by flash silica gel chromatography (40 g SepaFlash® Silica Flash Column, Eluent of 0-100% EtOAc/Petroleum ether to 0-30% DCM:MeOH gradient @ 100 mL/min) to afford benzyl 4-(((1R,5S,6s)-6-((tert-butoxycarbonyl)amino)-3-azabicyclo[3.1.0]hexan-3-yl)methyl) piperidine-1-carboxylate (1.72 g, 3.80 mmol, 81.80% yield, 95% purity) as a colorless oil. MS(M+H)⁺=430.2

### Step 2. Synthesis of tert-butyl ((1R,5S,6s)-3-(piperidin-4-ylmethyl)-3-azabicyclo[3.1.0]hexan-6-yl)carbamate (4)

To a solution of benzyl 4-(((1R,5S,6s)-6-((tert-butoxycarbonyl)amino)-3-azabicyclo[3.1.0]hexan-3-yl)methyl) piperidine-1-carboxylate (1.72 g, 4.00 mmol) in EtOH (20 mL) was added Pd/C (0.8 g, 10% purity) under N₂ atmosphere. The suspension was degassed and purged with H₂ for 3 times. The mixture was stirred under H₂ (15 Psi) at 20° C. for 12 h. LCMS showed the starting material was consumed completely and desired mass was detected. The reaction mixture was filtered. The filter cake was washed with EtOH (30 ml×3). The filtrate was concentrated in vacuum to afford tert-butyl ((1R,5S,6s)-3-(piperidin-4-ylmethyl)-3-azabicyclo[3.1.0]hexan-6-yl)carbamate (1.15 g, crude) as a gray solid. MS(M+H)⁺=296.3

### Step 3. Synthesis of tert-butyl ((1R,5S,6s)-3-((1-(4-nitrophenyl)piperidin-4-yl)methyl)-3-azabicyclo[3.1.0]hexan-6-yl)carbamate (5)

It was synthesized by similar method described in step 5 of Example 4, changed Acetone to DMF (20 mL) to obtain tert-butyl ((1R,5S,6s)-3-((1-(4-nitrophenyl)piperidin-4-yl)methyl)-3-azabicyclo[3.1.0]hexan-6-yl)carbamate (1.2 g, 2.65 mmol, 68.09% yield, 92% purity) as a yellow solid. MS(M+H)⁺=417.3

### Step 4. Synthesis of tert-butyl ((1R,5S,6s)-3-((1-(4-aminophenyl)piperidin-4-yl)methyl)-3-azabicyclo[3.1.0]hexan-6-yl)carbamate (6)

To a solution of tert-butyl ((1R,5S,6s)-3-((1-(4-nitrophenyl)piperidin-4-yl)methyl)-3-azabicyclo[3.1.0]hexan-6-yl)carbamate (1.2 g, 2.88 mmol) in EtOH (10 mL) and H₂O (10 mL) were added Fe (804.47 mg, 14.41 mmol) and NH₄Cl (462.34 mg, 8.64 mmol) at 20° C. The mixture was stirred at 80° C. for 2 h. LCMS showed the starting material was consumed completely and desired mass (Rₜ=0.200 min) was detected. The reaction mixture was concentrated in vacuum to give a residue. The residue was diluted with H₂O (50 mL) and adjusted to pH=10 with Na₂CO₃, extracted with EtOAc (20 mL×3). The combined organic layers were dried over Na₂SO₄ and concentrated in vacuum to afford tert-butyl ((1R,5S,6s)-3-((1-(4-aminophenyl)piperidin-4-yl)methyl)-3-azabicyclo[3.1.0]hexan-6-yl)carbamate (950 mg, 2.33 mmol, 81.04% yield, 95% purity) as a yellow solid. MS(M+H)⁺=387.3

### Step 5. Synthesis of tert-butyl ((1R,5S,6s)-3-((1-(4-((2,6-dioxopiperidin-3-yl)amino)phenyl)piperidin-4-yl)methyl)-3-azabicyclo[3.1.0]hexan-6-yl)carbamate (7)

To a solution of tert-butyl ((1R,5S,6s)-3-((1-(4-aminophenyl)piperidin-4-yl)methyl)-3-azabicyclo[3.1.0]hexan-6-yl)carbamate (900 mg, 2.33 mmol) and 3-bromopiperidine-2,6-dione (625.91 mg, 3.26 mmol) in ACN (4 mL) was added NaHCO₃ (978.01 mg, 11.64 mmol, 452.99 μL) at 20° C. The mixture was stirred at 80° C. for 16 h. LCMS showed tert-butyl ((1R,5S,6s)-3-((1-(4-aminophenyl)piperidin-4-yl)methyl)-3-azabicyclo[3.1.0]hexan-6-yl)carbamate was consumed completely and desired mass was detected. The reaction mixture was filtered. The filter cake was washed by mixture solution (40 mL, MeOH:EtOAc=1:1). The filtrate was concentrated in vacuum to give a residue. The residue was purified by flash silica gel chromatography (20 g SepaFlash® Silica Flash Column, Eluent of 0-100% EtOAc/Petroleum ether to 0-50% DCM:MeOH gradient @ 80 mL/min) and re-purified by prep-HPLC (column: Phenomenex luna C18 150*40 mm*15 μm; mobile phase: [water (TFA)-ACN]; gradient: 0%-30% B over 10 min) followed by lyophilization to afford tert-butyl ((1R,5S,6s)-3-((1-(4-((2,6-dioxopiperidin-3-yl)amino)phenyl)piperidin-4-yl)methyl)-3-azabicyclo[3.1.0]hexan-6-yl)carbamate (220 mg, 397.89 μmol, 17.09% yield, 90% purity) as a brown solid. MS(M+H)⁺=498.2

### Step 6. Synthesis of 3-((4-(4-(((1R,5S,6s)-6-amino-3-azabicyclo[3.1.0]hexan-3-yl)methyl)piperidin-1-yl) phenyl)amino)piperidine-2,6-dione (8)

It was synthesized by similar method described in step 5 of Example 1 to obtain 3-((4-(4-(((1R,5S,6s)-6-amino-3-azabicyclo[3.1.0]hexan-3-yl)methyl)piperidin-1-yl)phenyl)amino)piperidine-2,6-dione (200 mg, crude, HCl) as a yellow solid. MS(M+H)⁺=398.2

### Step 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-3-((1-(4-((2,6-dioxopiperidin-3-yl)amino) phenyl)piperidin-4-yl)methyl)-3-azabicyclo[3.1.0]hexan-6-yl)-3-methoxybenzamide (Compound 22)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-3-((1-(4-((2,6-dioxopiperidin-3-yl)amino)phenyl)piperidin-4-yl)methyl)-3-azabicyclo[3.1.0]hexan-6-yl)-3-methoxybenzamide (68.7 mg, 60.49 μmol, 21.88% yield, 90.6% purity, 2TFA) as a gray solid. MS(M+H)⁺=801.2

¹H NMR (400 MHz, DMSO-d₆) δ=10.85 (s, 1H), 9.57-9.27 (m, 1H), 8.55-8.45 (m, 1H), 8.29 (d, J=8.2 Hz, 1H), 8.23 (s, 1H), 8.06 (s, 1H), 7.50-7.46 (m, 2H), 7.43-7.21 (m, 2H), 6.80-6.73 (m, 2H), 4.91-4.84 (m, 1H), 4.43-4.34 (m, 2H), 4.06 (t, J=13.3 Hz, 2H), 3.93 (s, 3H), 3.89-3.83 (m, 2H), 3.57-3.52 (m, 2H), 3.49-3.43 (m, 2H), 3.32 (s, 3H), 3.22-3.17 (m, 1H), 3.13-3.01 (m, 1H), 2.99-2.89 (m, 1H), 2.81-2.69 (m, 1H), 2.63-2.55 (m, 1H), 2.41-2.37 (m, 1H), 2.16-2.15 (m, 1H), 2.12-2.03 (m, 5H), 1.97-1.88 (m, 2H), 1.63-1.57 (m, 1H), 1.24 (d, J=6.6 Hz, 6H).

### Example 23. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-3-(2-(4-(4-((2,6-dioxopiperidin-3-yl)amino)phenyl)piperazin-1-yl)ethyl)-3-azabicyclo[3.1.0]hexan-6-yl)-3-methoxybenzamide (Compound 23)

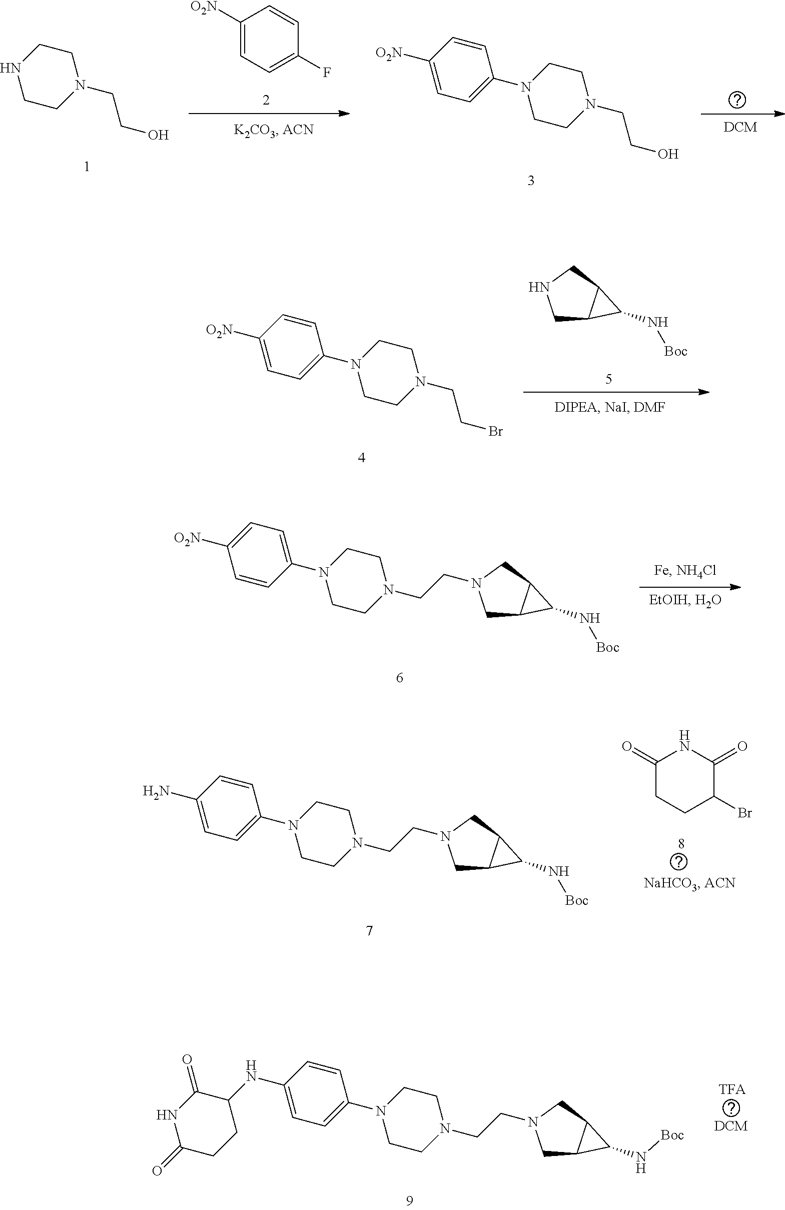

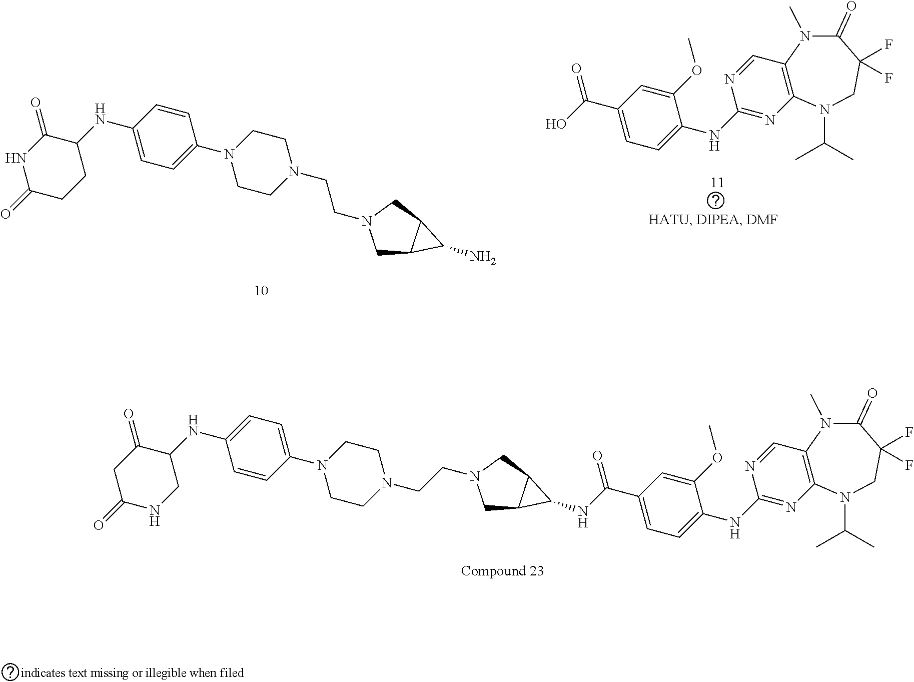 [SMILES: N[C@@H]1[C@H]2CN(CCN3CCN(c4ccc(NC5CCC(=O)NC5=O)cc4)CC3)C[C@@H]12 ; COc1cc(C(=O)O)ccc1Nc1ncc2c(n1)N(C(C)C)CC(F)(F)C(=O)N2C ; COc1cc(C(=O)N[C@H]2[C@@H]3CN(CCN4CCN(c5ccc(NC6CNC(=O)CC6=O)cc5)CC4)C[C@@H]32)ccc1Nc1ncc2c(n1)N(C(C)C)CC(F)(F)C(=O)N2C]

### Step 1. Synthesis of 2-(4-(4-nitrophenyl)piperazin-1-yl)ethan-1-ol (3)

It was synthesized by similar method described in step 6 of Example 4, changed Acetone to ACN to obtain 2-(4-(4-nitrophenyl)piperazin-1-yl)ethan-1-ol (1 g, 3.98 mmol, 34.54% yield, 100% purity) as a yellow solid. MS(M+H)⁺=252.2.

### Step 2. Synthesis of 1-(2-bromoethyl)-4-(4-nitrophenyl)piperazine (4)

To a solution of 2-(4-(4-nitrophenyl)piperazin-1-yl)ethan-1-ol (1 g, 3.98 mmol) in DCM (20 mL) were added PPh₃ (1.36 g, 5.17 mmol) and CBr₄ (1.72 g, 5.17 mmol) at 0° C. and the mixture was stirred at 25° C. for 16 h under N₂ atmosphere. LCMS showed a peak (32%) with desired mass. The mixture was filtered and concentrated. The residue was purified by flash silica gel chromatography (ISCO®; 40 g SepaFlash® Silica Flash Column, Eluent of 0-50% EtOAc/Petroleum ether gradient @ 80 mL/min) to afford 1-(2-bromoethyl)-4-(4-nitrophenyl)piperazine (0.6 g, 1.81 mmol, 45.59% yield, 95% purity) as a yellow solid. MS(M+H)⁺=314.2.

### Step 3. Synthesis of tert-butyl ((1R,5S,6s)-3-(2-(4-(4-nitrophenyl)piperazin-1-yl)ethyl)-3-azabicyclo[3.1.0]hexan-6-yl)carbamate (6)

To a solution of 1-(2-bromoethyl)-4-(4-nitrophenyl)piperazine (522.94 mg, 1.66 mmol) in DMF (10 mL) were added tert-butyl ((1R,5S,6s)-3-azabicyclo[3.1.0]hexan-6-yl)carbamate (300 mg, 1.51 mmol), DIPEA (667.78 mg, 5.17 mmol, 899.98 μL) and NaI (45.36 mg, 302.63 μmol), then the mixture was stirred at 20° C. for 16 h under N₂ atmosphere. LCMS showed a peak (81%) with desired mass. The reaction was diluted with water (20 mL) and extracted with EtOAc (20 mL×3). The combined organic layers were washed with saturated brine (50 mL), dried over anhydrous Na₂SO₄, filtered and concentrated under reduced pressure. The residue was purified by flash silica gel chromatography (ISCO®; 10 g SepaFlash® Silica Flash Column, Eluent of 0-100% EtOAc/Petroleum ether gradient @ 80 mL/min) to afford tert-butyl ((1R,5S,6s)-3-(2-(4-(4-nitrophenyl)piperazin-1-yl)ethyl)-3-azabicyclo[3.1.0]hexan-6-yl)carbamate (0.5 g, 1.16 mmol, 76.57% yield, 100% purity) as a yellow solid. MS(M+H)⁺=432.3.

### Step 4. Synthesis of tert-butyl ((1R,5S,6s)-3-(2-(4-(4-aminophenyl)piperazin-1-yl)ethyl)-3-azabicyclo[3.1.0]hexan-6-yl)carbamate (7)

It was synthesized by similar method described in step 4 of Example 22 to obtain tert-butyl ((1R,5S,6s)-3-(2-(4-(4-aminophenyl)piperazin-1-yl)ethyl)-3-azabicyclo[3.1.0]hexan-6-yl)carbamate (0.25 g, 579.01 μmol, 49.97% yield, 93% purity) as yellow oil. MS(M+H)⁺=402.3.

### Step 5. Synthesis of tert-butyl ((1R,5S,6s)-3-(2-(4-(4-((2,6-dioxopiperidin-3-yl)amino)phenyl)piperazin-1-yl)ethyl)-3-azabicyclo[3.1.0]hexan-6-yl)carbamate (9)

It was synthesized by similar method described in step 5 of Example 22 to obtain tert-butyl ((1R,5S,6s)-3-(2-(4-(4-((2,6-dioxopiperidin-3-yl)amino)phenyl)piperazin-1-yl)ethyl)-3-azabicyclo[3.1.0]hexan-6-yl)carbamate (120 mg, 215.35 μmol, 34.59% yield, 92% purity) as a black solid. MS(M+H)⁺=513.4.

### Step 6. Synthesis of 3-((4-(4-(2-((1R,5S,6s)-6-amino-3-azabicyclo[3.1.0]hexan-3-yl)ethyl)piperazin-1-yl)phenyl)amino)piperidine-2,6-dione (10)

It was synthesized by similar method described in step 6 of Example 3 to obtain 3-((4-(4-(2-((1R,5S,6s)-6-amino-3-azabicyclo[3.1.0]hexan-3-yl)ethyl)piperazin-1-yl)phenyl)amino)piperidine-2,6-dione (0.1 g, 189.92 μmol, 97.36% yield, TFA) as a red solid. MS(M+H)⁺=413.2

### Step 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-3-(2-(4-(4-((2,6-dioxopiperidin-3-yl)amino)phenyl)piperazin-1-yl)ethyl)-3-azabicyclo[3.1.0]hexan-6-yl)-3-methoxybenzamide (Compound 23)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-3-(2-(4-(4-((2,6-dioxopiperidin-3-yl)amino)phenyl)piperazin-1-yl)ethyl)-3-azabicyclo[3.1.0]hexan-6-yl)-3-methoxybenzamide (55.6 mg, 64.74 μmol, 34.09% yield, 95% purity) as a white solid. MS(M+H)⁺=816.4.

¹H NMR (400 MHz, DMSO-d₆) δ=10.76 (s, 1H), 8.29 (d, J=8.9 Hz, 1H), 8.26-8.20 (m, 2H), 7.88 (s, 1H), 7.50-7.43 (m, 2H), 6.75 (d, J=8.9 Hz, 2H), 6.61 (d, J=8.9 Hz, 2H), 5.37 (d, J=7.3 Hz, 1H), 4.93-4.82 (m, 1H), 4.25-4.14 (m, 1H), 4.04 (br t, J=13.6 Hz, 2H), 3.93 (s, 3H), 3.31 (br s, 3H), 3.12 (d, J=8.8 Hz, 2H), 3.06-3.00 (m, 1H), 2.99-2.90 (m, 4H), 2.79-2.66 (m, 2H), 2.64-2.56 (m, 4H), 2.46-2.40 (m, 3H), 2.39-2.30 (m, 3H), 2.17-2.05 (m, 1H), 1.92-1.76 (m, 1H), 1.68-1.61 (m, 2H), 1.24 (d, J=6.8 Hz, 6H).

### Example 24. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-3-(2-(1-(4-((2,6-dioxopiperidin-3-yl)amino)phenyl)piperidin-4-yl)ethyl)-3-azabicyclo[3.1.0]hexan-6-yl)-3-methoxybenzamide (Compound 24)

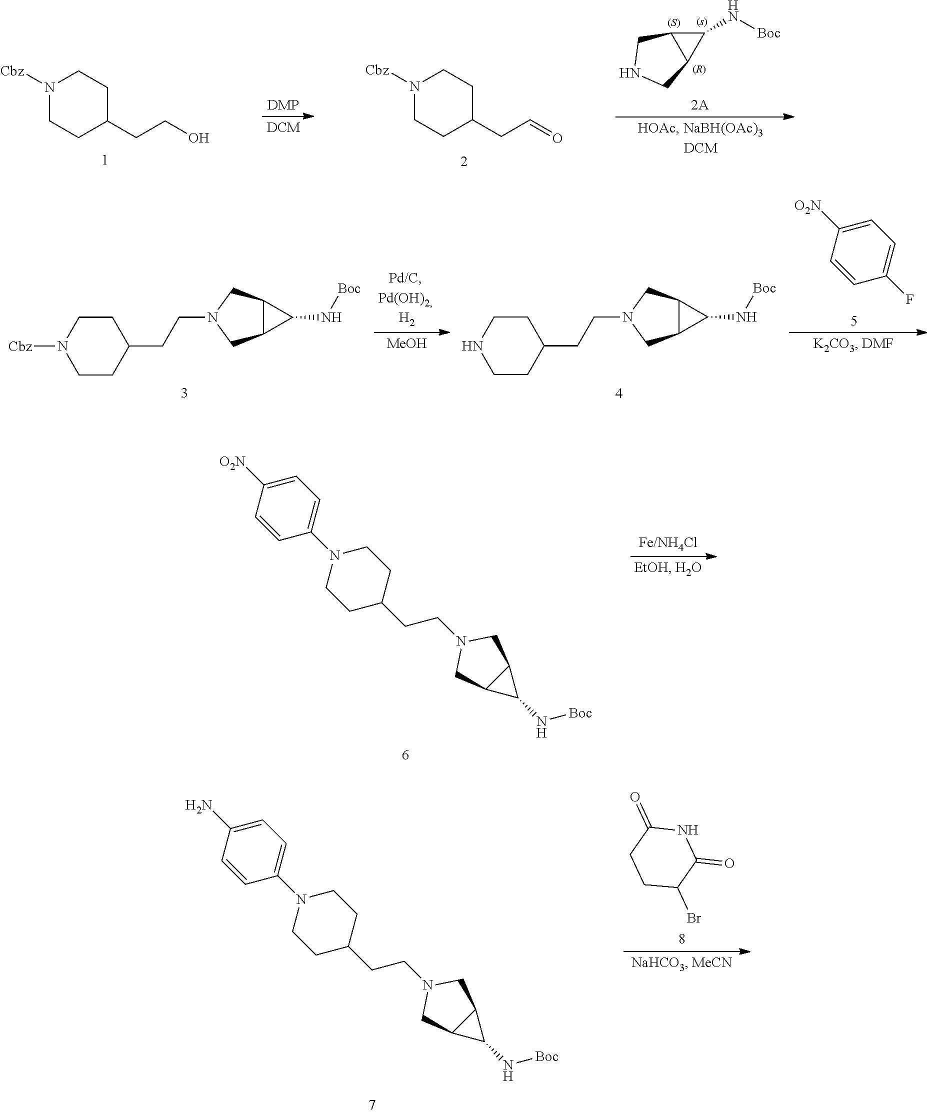

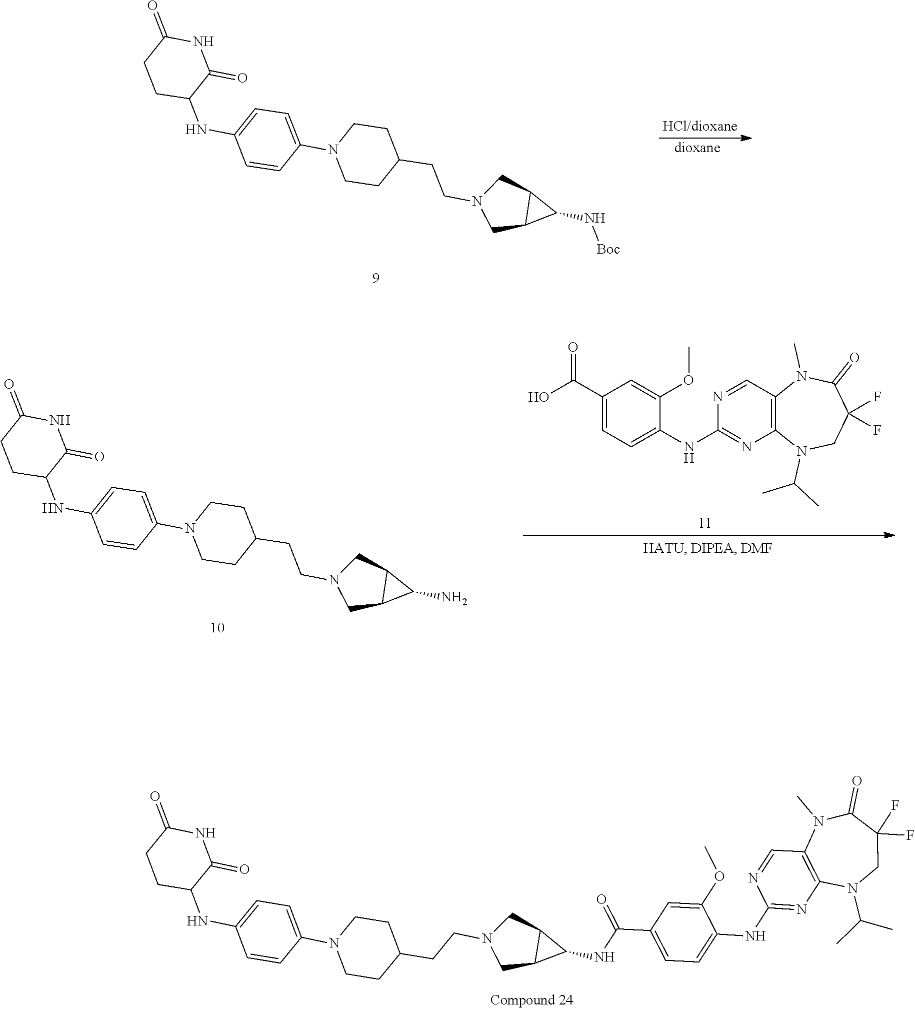 [SMILES: CC(C)(C)OC(=O)N[C@H]1[C@@H]2CN(CCC3CCN(c4ccc(NC5CCC(=O)NC5=O)cc4)CC3)C[C@@H]21 ; N[C@@H]1[C@H]2CN(CCC3CCN(c4ccc(NC5CCC(=O)NC5=O)cc4)CC3)C[C@@H]12 ; COc1cc(C(=O)O)ccc1Nc1ncc2c(n1)N(C(C)C)CC(F)(F)C(=O)N2C ; COc1cc(C(=O)N[C@H]2[C@@H]3CN(CCC4CCN(c5ccc(NC6CCC(=O)NC6=O)cc5)CC4)C[C@@H]32)ccc1Nc1ncc2c(n1)N(C(C)C)CC(F)(F)C(=O)N2C]

### Step 1. Synthesis of benzyl 4-(2-oxoethyl)piperidine-1-carboxylate (2)

It was synthesized by similar method described in step 1 of Example 6 to obtain benzyl 4-(2-oxoethyl)piperidine-1-carboxylate (1.94 g, 3.71 mmol, 97.75% yield, 50% purity) as a white solid. MS(M+H)⁺=262.2.

### Step 2. Synthesis of benzyl 4-(2-((1R,5S,6s)-6-((tert-butoxycarbonyl)amino)-3-azabicyclo[3.1.0]hexan-3-yl)eth yl)piperidine-1-carboxylate (3)

It was synthesized by similar method described in step 1 of Example 22 to obtain benzyl 4-(2-((1R,5S,6s)-6-((tert-butoxycarbonyl)amino)-3-azabicyclo[3.1.0]hexan-3-yl)ethyl) piperidine-1-carboxylate (1.4 g, crude) as a yellow oil. MS(M+H)⁺=444.3.

### Step 3. Synthesis of tert-butyl ((1R,5S,6s)-3-(2-(piperidin-4-yl)ethyl)-3-azabicyclo[3.1.0]hexan-6-yl)carbamate (4)

It was synthesized by similar method described in step 2 of Example 22 to obtain tert-butyl ((1R,5S,6s)-3-(2-(piperidin-4-yl)ethyl)-3-azabicyclo[3.1.0]hexan-6-yl)carbamate (1.1 g, crude) as a light yellow oil. MS(M+H)⁺=310.2.

### Step 4. Synthesis of tert-butyl ((1R,5S,6s)-3-(2-(1-(4-nitrophenyl)piperidin-4-yl)ethyl)-3-azabicyclo[3.1.0]hexan-6-yl)carbamate (6)

It was synthesized by similar method described in step 3 of Example 22 to obtain tert-butyl ((1R,5S,6s)-3-(2-(1-(4-nitrophenyl)piperidin-4-yl)ethyl)-3-azabicyclo[3.1.0]hexan-6-yl)carbamate (650 mg, 1.51 mmol, 42.47% yield) as a yellow solid. MS(M+H)⁺=431.2.

### Step 5. Synthesis of tert-butyl ((1R,5S,6s)-3-(2-(1-(4-aminophenyl)piperidin-4-yl)ethyl)-3-azabicyclo[3.1.0]hexan-6-yl)carbamate (7)

It was synthesized by similar method described in step 4 of Example 22 to obtain tert-butyl ((1R,5S,6s)-3-(2-(1-(4-aminophenyl)piperidin-4-yl)ethyl)-3-azabicyclo[3.1.0]hexan-6-yl)carbamate (650 mg, 1.62 mmol, 88.44% yield) as a gray solid. MS(M+H)⁺=401.3.

### Step 6. Synthesis of tert-butyl ((1R,5S,6s)-3-(2-(1-(4-((2,6-dioxopiperidin-3-yl)amino)phenyl)piperidin-4-yl)ethyl)-3-azabicyclo[3.1.0]hexan-6-yl)carbamate (9)

It was synthesized by similar method described in step 5 of Example 22 to obtain tert-butyl ((1R,5S,6s)-3-(2-(1-(4-((2,6-dioxopiperidin-3-yl)amino)phenyl)piperidin-4-yl)ethyl)-3-azabicyclo[3.1.0]hexan-6-yl)carbamate (240 mg, 469.07 μmol, 93.94% yield) as a gray solid. MS(M+H)⁺=512.4.

### Step 7. Synthesis of 3-((4-(4-(2-((1R,5S,6s)-6-amino-3-azabicyclo[3.1.0]hexan-3-yl)ethyl)piperidin-1-yl) phenyl)amino)piperidine-2,6-dione (10)

It was synthesized by similar method described in step 6 of Example 22 to obtain 3-((4-(4-(2-((1R,5S,6s)-6-amino-3-azabicyclo[3.1.0]hexan-3-yl)ethyl)piperidin-1-yl)phenyl)amino)piperidine-2,6-dione (200 mg, crude, HCl salt) as a blue solid. MS(M+H)⁺=412.3.

### Step 8. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-3-(2-(1-(4-((2,6-dioxopiperidin-3-yl)amino)phenyl)piperidin-4-yl)ethyl)-3-azabicyclo[3.1.0]hexan-6-yl)-3-methoxybenzamide (Compound 24)

It was synthesized by similar method described in step 7 of Example 22 to obtain 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-3-(2-(1-(4-((2,6-dioxopiperidin-3-yl)amino)phenyl)piperidin-4-yl)ethyl)-3-azabicyclo[3.1.0]hexan-6-yl)-3-methoxybenzamide (78.9 mg, 91.30 μmol, 24.66% yield, 94.3% purity) as a gray solid. MS(M+H)⁺=815.5.

¹H NMR (400 MHz, DMSO-d₆) δ=10.75 (s, 1H), 8.30-8.27 (m, 1H), 8.25-8.22 (m, 1H), 8.21 (s, 1H), 7.87 (s, 1H), 7.47-7.44 (m, 2H), 6.74 (d, J=8.9 Hz, 2H), 6.59 (d, J=8.9 Hz, 2H), 5.35 (d, J=7.3 Hz, 1H), 4.91-4.82 (m, 1H), 4.22-4.14 (m, 1H), 4.03 (t, J=13.6 Hz, 2H), 3.92 (s, 3H), 3.36 (s, 3H), 3.07 (d, J=8.8 Hz, 2H), 3.01-2.98 (m, 1H), 2.78-2.69 (m, 1H), 2.61-2.57 (m, 1H), 2.56-2.54 (m, 1H), 2.47-2.44 (m, 2H), 2.44-2.39 (m, 3H), 2.29 (d, J=7.9 Hz, 2H), 2.14-2.07 (m, 1H), 1.86-1.80 (m, 1H), 1.72 (d, J=11.3 Hz, 2H), 1.64-1.58 (m, 2H), 1.42-1.34 (m, 3H), 1.34-1.26 (m, 2H), 1.23 (d, J=6.8 Hz, 6H).

### Example 25. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-6-(2-(4-(4-((2,6-dioxopiperidin-3-yl)amino)phenyl)piperazin-1-yl)ethyl)-3-azabicyclo[3.1.0]hexan-3-yl)-3-methoxybenzamide (Compound 25)

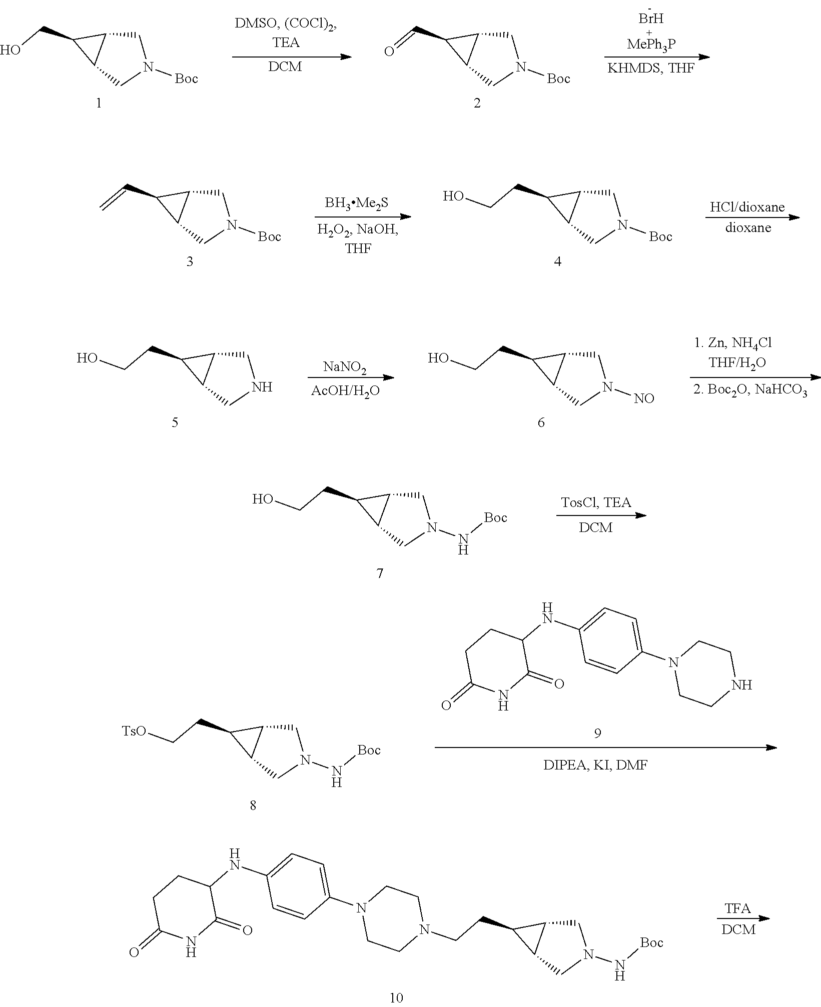

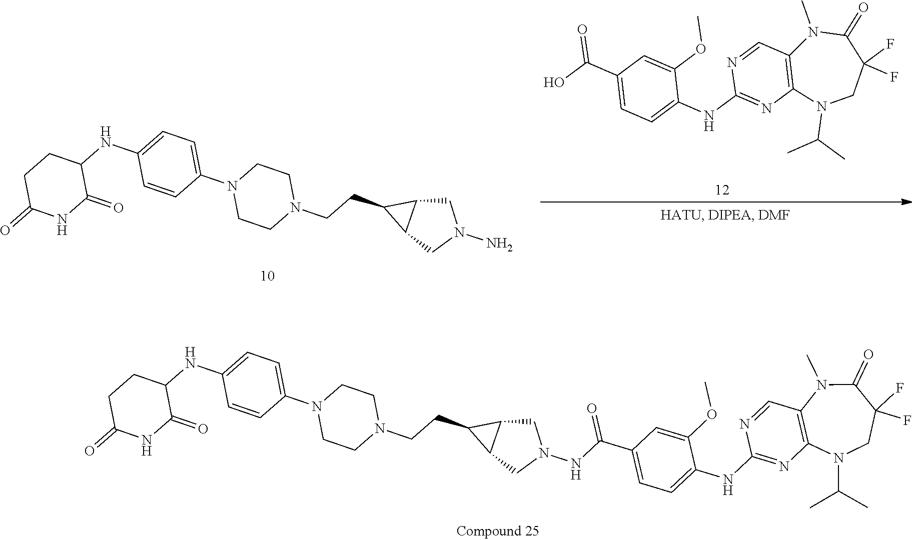 [SMILES: NN1C[C@@H]2[C@@H](CCN3CCN(c4ccc(NC5CCC(=O)NC5=O)cc4)CC3)[C@@H]2C1 ; COc1cc(C(=O)O)ccc1Nc1ncc2c(n1)N(C(C)C)CC(F)(F)C(=O)N2C ; COc1cc(C(=O)NN2C[C@@H]3[C@@H](CCN4CCN(c5ccc(NC6CCC(=O)NC6=O)cc5)CC4)[C@@H]3C2)ccc1Nc1ncc2c(n1)N(C(C)C)CC(F)(F)C(=O)N2C]

### Step 1. Synthesis of tert-butyl (1R,5S,6r)-6-formyl-3-azabicyclo[3.1.0]hexane-3-carboxylate (2)

To a solution of DMSO (5.50 g, 70.33 mmol, 5.50 mL) in DCM (50 mL) was slowly added (COCl)₂ (3.57 g, 28.13 mmol, 2.46 mL) at −70° C. and the mixture was stirred at −70° C. for 0.5 h. Then a solution of tert-butyl (1R,5S,6r)-6-(hydroxymethyl)-3-azabicyclo[3.1.0]hexane-3-carboxylate (3 g, 14.07 mmol) in DCM (5 mL) was added and the mixture was stirred at −70° C. for 0.5 h. TEA (7.12 g, 70.33 mmol, 9.79 mL) was added and the mixture was stirred at −70° C. for 0.5 h. TLC (Petroleum ether/EtOAc=2/1) showed that starting material was consumed and new spot formed. The residue was diluted with sat. aq. Na₂SO₃ (100 mL) and extracted with DCM (50 mL×3), the combined organic layers were washed with brine (100 mL), dried over anhydrous Na₂SO₄, filtered and concentrated under reduced pressure. The residue was purified by flash silica gel chromatography (40 g SepaFlash® Silica Flash Column, Eluent of 0-30% EtOAc/Petroleum ether gradient @80 mL/min) to afford tert-butyl (1R,5S,6r)-6-formyl-3-azabicyclo[3.1.0]hexane-3-carboxylate (2.7 g, 12.78 mmol, 90.86% yield) as yellow oil. MS(M+H)⁺=212.3.

### Step 2. Synthesis of tert-butyl (1R,5S,6s)-6-vinyl-3-azabicyclo[3.1.0]hexane-3-carboxylate (3)

To the suspension of methyl(triphenyl)phosphonium; bromide (6.85 g, 19.17 mmol) in THF (50 mL) was added KHMDS (1 M, 19.17 mL) at 0° C. under N₂, the mixture was stirred at 20° C. for 1 h, a solution of tert-butyl (1R,5S,6r)-6-formyl-3-azabicyclo[3.1.0]hexane-3-carboxylate (2.7 g, 12.78 mmol) in THF (2 mL) was added at −70° C. under N₂. The mixture was warmed to −10° C. and stirred at −10° C. for 1 h. TLC (Petroleum ether/EtOAc=2/1) showed that starting material was consumed and new spot was formed. The residue was diluted with sat. aq. NH₄Cl (50 mL) and extracted with EtOAc (30 mL×3), the combined organic layers were washed with brine (50 mL), dried over anhydrous Na₂SO₄, filtered and concentrated under reduced pressure. The residue was purified by flash silica gel chromatography (40 g SepaFlash® Silica Flash Column, Eluent of 0-20% EtOAc/Petroleum ether gradient @ 80 mL/min) to afford tert-butyl (1R,5S,6s)-6-vinyl-3-azabicyclo[3.1.0]hexane-3-carboxylate (2.4 g, 11.47 mmol, 89.73% yield) as yellow oil. MS(M+H)⁺=210.3.

### Step 3. Synthesis of tert-butyl (1R,5S,6s)-6-(2-hydroxyethyl)-3-azabicyclo[3.1.0]hexane-3-carboxylate (4)

To a solution of tert-butyl (1R,5S,6s)-6-vinyl-3-azabicyclo[3.1.0]hexane-3-carboxylate (2.4 g, 11.47 mmol) in THF (50 mL) cooled at 0° C. was added a solution of BH₃-Me₂S (10 M, 1.72 mL) under N₂, the mixture was stirred for 30 minutes at 0° C. The reaction was allowed to warm to 20° C. and stirred for 1 h. The mixture was treated carefully with NaOH (3 M, 9.60 mL) followed by the addition of H₂O₂ (6.80 g, 59.95 mmol, 5.76 mL, 30% purity) at 0° C. The resulting mixture was stirred at 65° C. for 1 h. TLC (Petroleum ether/EtOAc=2/1) showed starting material was consumed and new spot was formed. The mixture was quenched with the addition of saturated aqueous Na₂SO₃ (100 mL), adjusted pH=8 with 1N HCl, the resulting solution was extracted with EtOAc (50 mL×3), washed with brine (100 mL), dried over anhydrous Na₂SO₄ and concentrated under vacuum. The residue was purified by flash silica gel chromatography (40 g SepaFlash® Silica Flash Column, Eluent of 0-30% EtOAc/Petroleum ether gradient @ 100 mL/min) to afford tert-butyl (1R,5S,6s)-6-(2-hydroxyethyl)-3-azabicyclo[3.1.0]hexane-3-carboxylate (1.2 g, 5.28 mmol, 46.04% yield) as a white solid. MS(M+H)⁺=228.3.

### Step 4. Synthesis of 2-((1R,5S,6s)-3-azabicyclo[3.1.0]hexan-6-yl)ethan-1-ol (5)

It was synthesized by similar method described in step 5 of Example 1 to obtain 2-((1R,5S,6s)-3-azabicyclo[3.1.0]hexan-6-yl)ethan-1-ol (0.9 g, crude, HCl) as colorless oil. MS(M+H)⁺=128.2.

¹H NMR (400 MHz, CD₃OD) δ=3.64 (t, J=6.4 Hz, 2H), 3.42-3.37 (m, 4H), 1.71-1.62 (m, 2H), 1.53 (q, J=6.5 Hz, 2H), 0.97-0.87 (m, 1H).

### Step 5. Synthesis of 2-((1R,5S,6s)-3-nitroso-3-azabicyclo[3.1.0]hexan-6-yl)ethan-1-ol (6)

To the solution of 2-((1R,5S,6s)-3-azabicyclo[3.1.0]hexan-6-yl)ethan-1-ol (0.9 g, 5.50 mmol, HCl) in H₂O (20 mL) were added NaNO₂ (1.14 g, 16.50 mmol) and AcOH (1.32 g, 22.00 mmol, 1.26 mL) and the resulting mixture was stirred at 20° C. for 12 h. TLC (EtOAc) showed that starting material was consumed and new spot was formed. The residue was diluted with water (50 mL) and extracted with EtOAc (30 mL×3), the combined organic layers were washed with brine (50 mL), dried over anhydrous Na₂SO₄, filtered and concentrated under reduced pressure to afford 2-((1R,5S,6s)-3-nitroso-3-azabicyclo[3.1.0]hexan-6-yl)ethan-1-ol (1.2 g, crude) as yellow oil. MS(M+H)⁺=157.2.

### Step 6. Synthesis of tert-butyl ((1R,5S,6s)-6-(2-hydroxyethyl)-3-azabicyclo[3.1.0]hexan-3-yl)carbamate (7)

To a solution of 2-((1R,5S,6s)-3-nitroso-3-azabicyclo[3.1.0]hexan-6-yl)ethan-1-ol (0.6 g, 3.84 mmol) in THF (10 mL) and H₂O (2 mL) were added NH₄Cl (821.98 mg, 15.37 mmol) and Zn (1.02 g, 15.53 mmol) portion wise. The mixture was stirred at 20° C. for 2 h. The reaction mixture was filtered and filter cake was washed with THF (10 mL) and H₂O (10 mL). To the filtrate were added Boc₂O (2.52 g, 11.53 mmol, 2.65 mL) and NaHCO₃ (968.18 mg, 11.53 mmol) and the mixture was stirred at 20° C. for 12 h. TLC (EtOAc) showed that new spot was formed. The residue was diluted with water (30 mL) and extracted with EtOAc (20 mL×3), the combined organic layers were washed with brine (50 mL), dried over anhydrous Na₂SO₄, filtered and concentrated under reduced pressure. The residue was purified by flash silica gel chromatography (ISCO®; 5 g SepaFlash® Silica Flash Column, Eluent of 0-60% EtOAc/Petroleum ether gradient @ 50 mL/min) to afford tert-butyl ((1R,5S,6s)-6-(2-hydroxyethyl)-3-azabicyclo[3.1.0]hexan-3-yl)carbamate (0.2 g, 825.38 mol, 21.48% yield) as a white solid. MS(M+H)⁺=243.3.

¹H NMR (400 MHz, CDCl₃) δ=5.37 (br s, 1H), 3.74-3.65 (m, 3H), 3.20 (d, J=8.3 Hz, 2H), 2.89 (d, J=8.6 Hz, 2H), 1.49-1.36 (m, 11H), 1.26-1.18 (m, 3H).

### Step 7. Synthesis of 2-((1R,5S,6s)-3-((tert-butoxycarbonyl)amino)-3-azabicyclo[3.1.0]hexan-6-yl)ethyl 4-methylbenzenesulfonate (8)

To a solution of tert-butyl ((1R,5S,6s)-6-(2-hydroxyethyl)-3-azabicyclo[3.1.0]hexan-3-yl)carbamate (150 mg, 619.03 mol) in DCM (5 mL) were added TEA (187.92 mg, 1.86 mmol, 258.48 μL) and TosCl (141.62 mg, 742.84 μmol) at 20° C. and the mixture was stirred at 20° C. for 16 h. LCMS showed a peak (80%) with desired mass. The residue was filtered and concentrated under pressure. The residue was purified by flash silica gel chromatography (5 g SepaFlash® Silica Flash Column, Eluent of 0-40% EtOAc/Petroleum ether gradient @ 60 mL/min) to afford 2-((1R,5S,6s)-3-((tert-butoxycarbonyl)amino)-3-azabicyclo[3.1.0]hexan-6-yl)ethyl 4-methylbenzenesulfonate (130 mg, 327.87 μmol, 52.96% yield) as yellow oil. MS(M+Na)⁺=419.2

### Step 8. Synthesis of tert-butyl ((1R,5S,6s)-6-(2-(4-(4-((2,6-dioxopiperidin-3-yl)amino)phenyl)piperazin-1-yl)ethyl)-3-azabicyclo[3.1.0]hexan-3-yl)carbamate (10)

To a solution of 2-((1R,5S,6s)-3-((tert-butoxycarbonyl)amino)-3-azabicyclo[3.1.0]hexan-6-yl)ethyl 4-methylbenzenesulfonate (130 mg, 327.87 μmol) and 3-((4-(piperazin-1-yl)phenyl)amino)piperidine-2,6-dione (130.00 mg, 400.24 mol, HCl) in DMF (2 mL) were added KI (10.89 mg, 65.57 μmol) and DIPEA (127.12 mg, 983.61 μmol, 171.33 μL) and the mixture was stirred at 80° C. for 2 h. LCMS showed a main peak with desired mass. The reaction mixture was diluted with water (20 mL) and extracted with EtOAc (10 mL×3). The combined organic layers were washed with saturated brine (20 mL), dried over anhydrous Na₂SO₄, filtered and concentrated under reduced pressure. The residue was purified by flash silica gel chromatography (5 g SepaFlash® Silica Flash Column, Eluent of 0-10% MeOH/EtOAc gradient @ 60 mL/min) to afford tert-butyl ((1R,5S,6s)-6-(2-(4-(4-((2,6-dioxopiperidin-3-yl)amino)phenyl)piperazin-1-yl)ethyl)-3-azabicyclo[3.1.0]hexan-3-yl)carbamate (80 mg, 131.09 mol, 39.98% yield, 84% purity) as yellow oil. MS(M+H)⁺=513.4

### Step 9. Synthesis of 3-((4-(4-(2-((1R,5S,6s)-3-amino-3-azabicyclo[3.1.0]hexan-6-yl)ethyl)piperazin-1-yl)phenyl)amino)piperidine-2,6-dione (11)

It was synthesized by similar method described in step 6 of Example 3 to obtain 3-((4-(4-(2-((1R,5S,6s)-3-amino-3-azabicyclo[3.1.0]hexan-6-yl)ethyl)piperazin-1-yl)phenyl)amino)piperidine-2,6-dione (80 mg, 151.93 mol, 97.36% yield, TFA) as yellow oil. MS(M+H)⁺=413.3

### Step 10. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-6-(2-(4-(4-((2,6-dioxopiperidin-3-yl)amino)phenyl)piperazin-1-yl)ethyl)-3-azabicyclo[3.1.0]hexan-3-yl)-3-methoxybenzamide (Compound 25)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-((1R,5S,6s)-6-(2-(4-(4-((2,6-dioxopiperidin-3-yl)amino)phenyl)piperazin-1-yl)ethyl)-3-azabicyclo[3.1.0]hexan-3-yl)-3-methoxybenzamide (13.7 mg, 15.28 mol, 10.06% yield, 91% purity) as a brown solid. MS(M+H)⁺=816.4.

¹H NMR (400 MHz, DMSO-d₆) δ=10.81 (s, 1H), 9.33 (s, 1H), 8.35 (d, J=9.0 Hz, 1H), 8.28 (s, 1H), 7.93 (s, 1H), 7.51-7.41 (m, 2H), 6.82 (d, J=8.8 Hz, 2H), 6.67 (d, J=8.8 Hz, 2H), 5.44 (d, J=6.5 Hz, 1H), 5.01-4.83 (m, 1H), 4.30-4.21 (m, 1H), 4.09 (t, J=13.7 Hz, 2H), 3.98 (s, 3H), 3.51-3.42 (m, 5H), 3.39-3.31 (m, 3H), 3.31-2.56 (m, 2H), 3.19-3.06 (m, 2H), 3.12-3.03 (m, 4H), 2.85-2.75 (m, 1H), 2.69-2.61 (m, 2H), 2.21-2.12 (m, 1H), 1.95-1.82 (m, 1H), 1.47-1.40 (m, 2H), 1.35-1.29 (m, 8H), 1.18-1.12 (m, 1H).

### Example 26. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(4-((2,6-dioxopiperidin-3-yl)amino)-2-fluorophenyl)piperidin-4-yl)methyl)(methyl)amino)cyclohexyl)-3-methoxybenzamide (Compound 26)

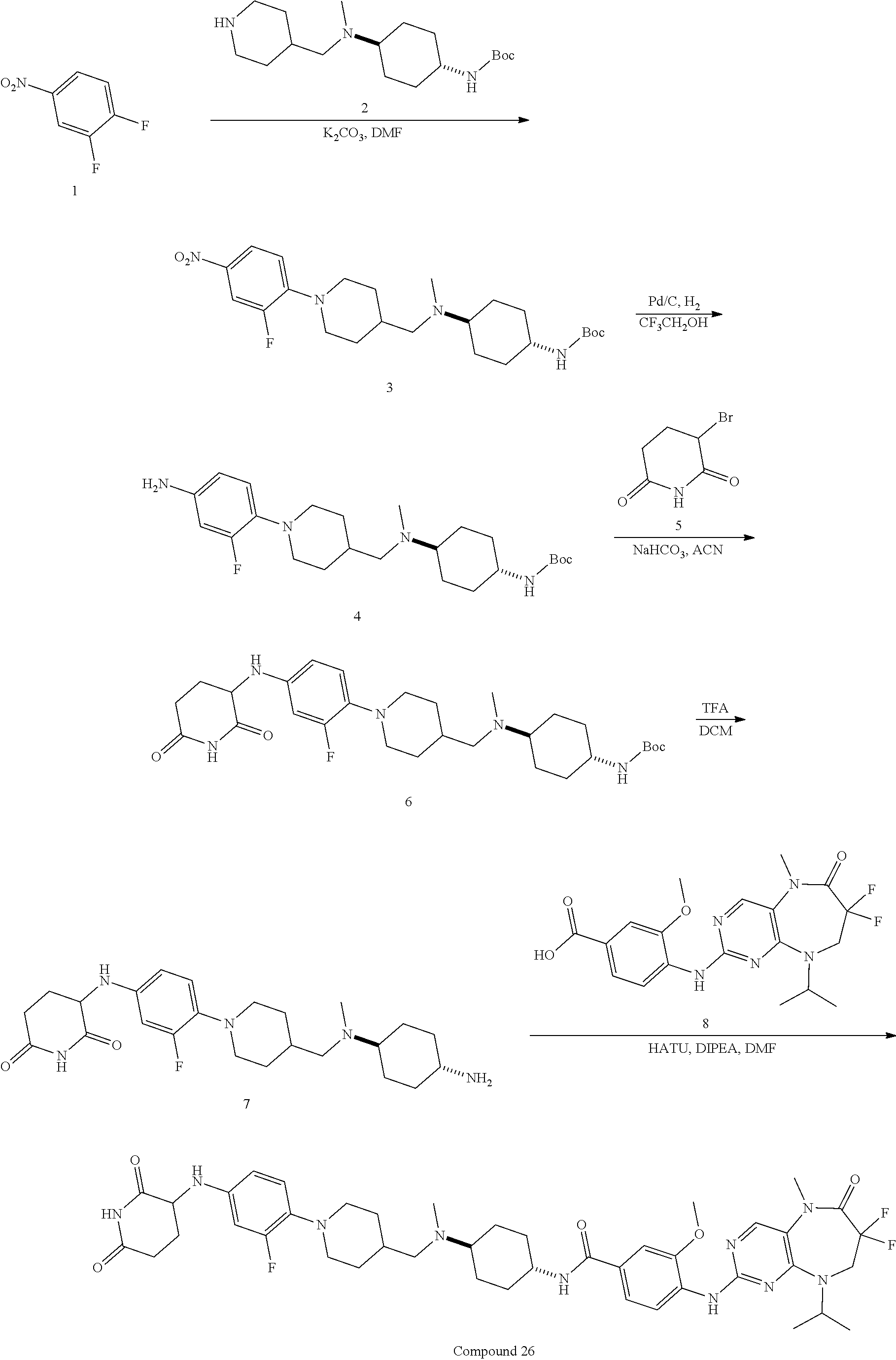

### Step 1. Synthesis of tert-butyl ((1r,4r)-4-(((1-(2-fluoro-4-nitrophenyl)piperidin-4-yl)methyl)(methyl)amino)cyclo hexyl)carbamate (3)

It was synthesized by similar method described in step 6 of Example 4, changed Acetone to DMF (50 mL) to obtain tert-butyl ((1r,4r)-4-(((1-(2-fluoro-4-nitrophenyl)piperidin-4-yl)methyl)(methyl)amino)cyclohex yl)carbamate (3 g, 6.07 mmol, 79.03% yield, 94% purity) as a yellow solid. MS(M+H)⁺=465.4.

### Step 2. Synthesis of tert-butyl ((1r,4r)-4-(((1-(4-amino-2-fluorophenyl)piperidin-4-yl)methyl)(methyl)amino)cyclohexyl)carbamate (4)

To a solution of tert-butyl ((1r,4r)-4-(((1-(2-fluoro-4-nitrophenyl)piperidin-4-yl)methyl)(methyl)amino)cyclohex yl)carbamate (3 g, 6.46 mmol) in CF₃CH₂OH (100 mL) was added Pd/C (500 mg, 10% purity) slowly under N₂ and the suspension was degassed and purged H₂ for 3 times, then the mixture was stirred at 25° C. for 14 h under H₂ (15 Psi). TLC (DCM:MeOH=10:1) showed the starting material was consumed and the new spot was detected. The mixture was filtered and the filter cake was washed with EtOH (100 mL) and THF (100 mL). The filtrate was concentrated under reduced pressure to afford tert-butyl ((1r,4r)-4-(((1-(4-amino-2-fluorophenyl)piperidin-4-yl)methyl)(methyl)amino)cyclohexyl)carbamate (2.8 g, crude) as a yellow oil. MS(M+H)⁺=435.4.

### Step 3. Synthesis of tert-butyl ((1r,4r)-4-(((1-(4-((2,6-dioxopiperidin-3-yl)amino)-2-fluorophenyl)piperidin-4-yl) methyl)(methyl)amino)cyclohexyl)carbamate (6)

It was synthesized by similar method described in step 5 of Example 22 to obtain tert-butyl ((1r,4r)-4-(((1-(4-((2,6-dioxopiperidin-3-yl)amino)-2-fluorophenyl)piperidin-4-yl)meth yl)(methyl)amino)cyclohexyl)carbamate (540 mg, 761.97 mol, 33.11% yield, 77% purity) as a blue solid and tert-butyl ((1r,4r)-4-(((1-(4-((2,6-dioxopiperidin-3-yl)amino)-2-fluorophenyl)piperidin-4-yl)meth yl)(methyl)amino)cyclohexyl)carbamate (420 mg, 677.31 mol, 29.44% yield, 88% purity) as a blue solid. MS(M+H)⁺=546.5.

### Step 4. Synthesis of 3-((4-(4-((((1r,4r)-4-aminocyclohexyl)(methyl)amino)methyl)piperidin-1-yl)-3-fluorophenyl)amino)piperidine-2,6-dione (7)

It was synthesized by similar method described in step 6 of Example 3 to obtain 3-((4-(4-((((1r,4r)-4-aminocyclohexyl)(methyl)amino)methyl)piperidin-1-yl)-3-fluorophenyl)amino)piperidine-2,6-dione (150 mg, crude, TFA salt) as a black brown oil. MS(M+H)⁺=446.5.

### Step 5. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(4-((2,6-dioxopiperidin-3-yl)amino)-2-fluorophenyl)piperidin-4-yl)methyl)(methyl)amino)cyclohexyl)-3-methoxybenzamide (Compound 26)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(4-((2,6-dioxopiperidin-3-yl)amino)-2-fluoro phenyl)piperidin-4-yl)methyl)(methyl)amino)cyclohexyl)-3-methoxybenzamide (29.8 mg, 34.05 μmol, 14.35% yield, 97% purity) as a gray solid. MS(M+H)⁺=849.4.

¹H NMR (400 MHz, DMSO-d₆) δ=10.78 (s, 1H), 8.30 (d, J=8.3 Hz, 1H), 8.22 (s, 1H), 8.12-8.07 (m, 1H), 7.88 (s, 1H), 7.52-7.46 (m, 2H), 6.83 (t, J=9.3 Hz, 1H), 6.53-6.46 (m, 1H), 6.43-6.38 (m, 1H), 5.82-5.75 (m, 1H), 4.92-4.83 (m, 1H), 4.29-4.20 (m, 1H), 4.09-3.99 (m, 2H), 3.93 (s, 3H), 3.81-3.70 (m, 1H), 3.34 (s, 3H), 3.15-3.07 (m, 2H), 2.78-2.68 (m, 1H), 2.60-2.52 (m, 9H), 2.11-2.04 (m, 1H), 1.96-1.89 (m, 3H), 1.86-1.72 (m, 4H), 1.47-1.34 (m, 4H), 1.29-1.17 (m, 9H).

### Example 27. Synthesis of N((1r,4r)-4-(((1-(4-((2,6-dioxopiperidin-3-yl)amino)-2-fluorophenyl)piperidin-4-yl) methyl)(methyl)amino)cyclohexyl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (Compound 27)

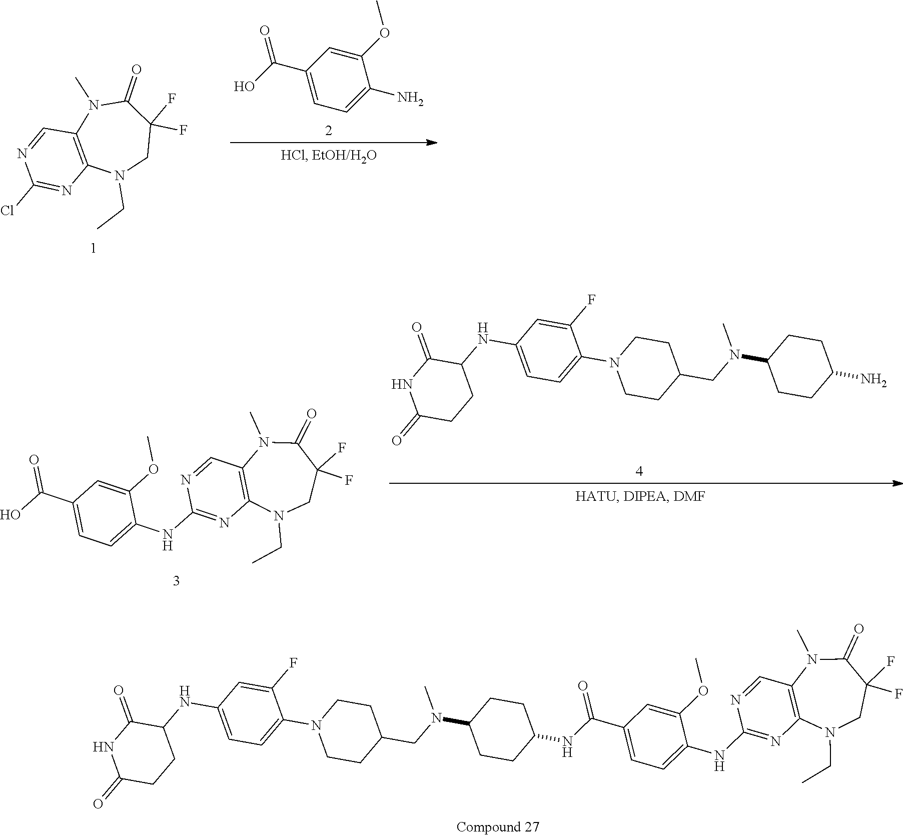

### Step 1. Synthesis of 4-((9-ethyl-7,7-difluoro-5-methyl-6-oxo-8H-pyrimido[4,5-b][1,4]diazepin-2-yl)amino)-3-methoxy-benzoic acid (3)

To a solution of 2-chloro-9-ethyl-7,7-difluoro-5-methyl-8H-pyrimido[4,5-b][1,4]diazepin-6-one (650 mg, 2.35 mmol) and 4-amino-3-methoxy-benzoic acid (400.58 mg, 2.40 mmol) in H₂O (16 mL) and EtOH (4 mL) was added HCl (12 M, 293.67 μL), the mixture was stirred at 100° C. for 16 h. LCMS showed a peak (79%) with desired mass. The reaction was filtered at 50° C. and filter cake was collected to afford 4-((9-ethyl-7,7-difluoro-5-methyl-6-oxo-8H-pyrimido[4,5-b][1,4]diazepin-2-yl)amino)-3-methoxy-benzoic acid (0.6 g, 1.44 mmol, 61.44% yield, 98% purity) as a brown solid. MS(M+H)⁺=408.1.

### Step 2. Synthesis of N((1r,4r)-4-(((1-(4-((2,6-dioxopiperidin-3-yl)amino)-2-fluorophenyl)piperidin-4-yl) methyl)(methyl)amino)cyclohexyl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (Compound 27)

It was synthesized by similar method described in step 10 of Example 2 to obtain N((1r,4r)-4-(((1-(4-((2,6-dioxopiperidin-3-yl)amino)-2-fluorophenyl)piperidin-4-yl)meth yl)(methyl)amino)cyclohexyl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (48.6 mg, 39.64 mol, 5.89% yield, 96% purity, 3TFA) as gray solid. MS(M+H)⁺=835.4.

¹H NMR (400 MHz, DMSO-d₆) δ=10.79 (s, 1H), 8.92-8.75 (m, 1H), 8.31-8.14 (m, 4H), 7.56-7.45 (m, 2H), 7.01-6.86 (m, 1H), 6.54 (d, J=15.7 Hz, 1H), 6.46 (d, J=7.9 Hz, 1H), 4.28-4.25 (m, 2H), 4.22-4.21 (m, 1H), 4.18-4.17 (m, 1H), 3.94 (s, 3H), 3.87-3.80 (m, 1H), 3.66 (q, J=6.9 Hz, 2H), 3.33 (s, 3H), 3.29-3.16 (m, 4H), 3.01-2.91 (m, 1H), 2.83-2.78 (m, 3H), 2.78-2.69 (m, 2H), 2.13-1.75 (m, 10H), 1.74-1.60 (m, 2H), 1.55-1.36 (m, 4H), 1.21 (t, J=7.0 Hz, 3H).

### Example 28. Synthesis of N(4-(2-(4-(4-((2,6-dioxopiperidin-3-yl)amino)-2-fluorophenyl)piperazin-1-yl)ethyl)piperidin-1-yl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (Compound 28)

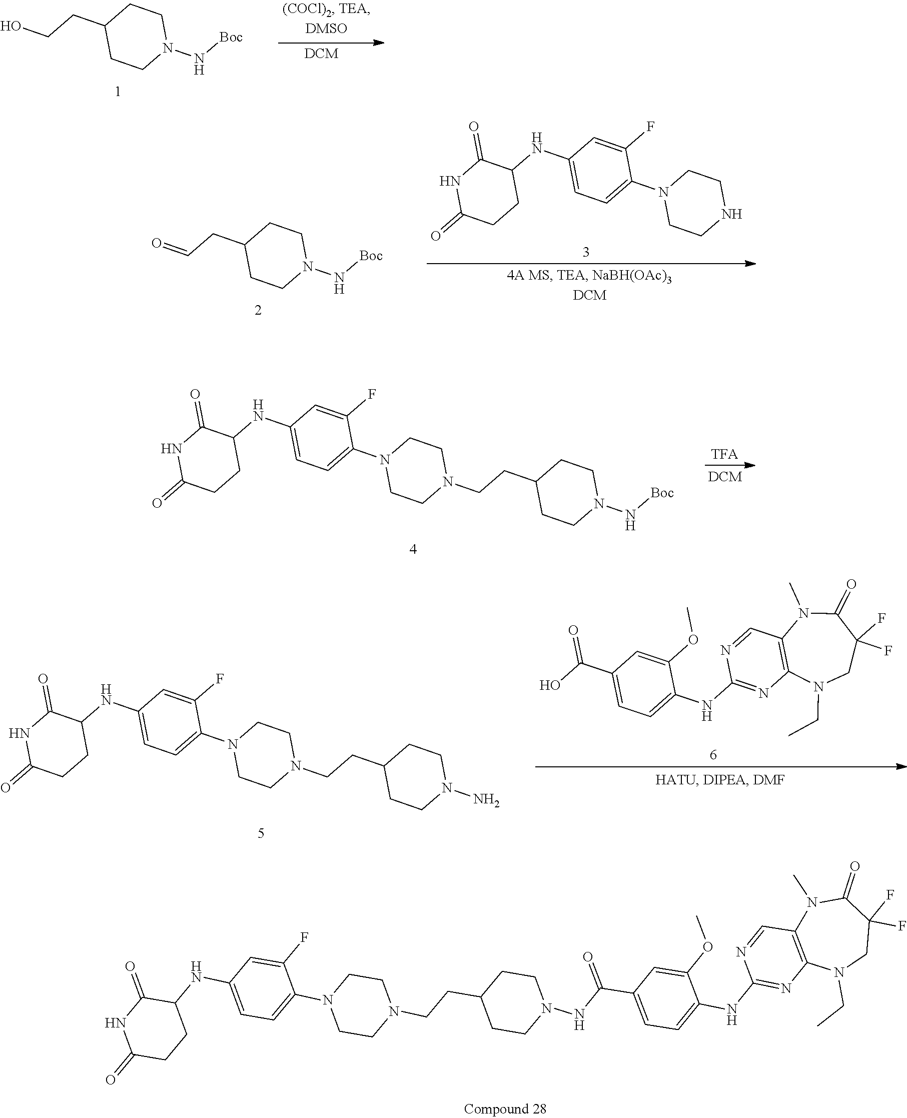

### Step 1. Synthesis of tert-butyl (4-(2-oxoethyl)piperidin-1-yl)carbamate (2)

It was synthesized by similar method described in step 1 of Example 25 to obtain tert-butyl (4-(2-oxoethyl)piperidin-1-yl)carbamate (2.5 g, crude) as yellow oil. MS(M+H)⁺=243.3

### Step 2. Synthesis of tert-butyl (4-(2-(4-(4-((2,6-dioxopiperidin-3-yl)amino)-2-fluorophenyl)piperazin-1-yl)ethyl)piperidin-1-yl)carbamate (4)

It was synthesized by similar method described in step 6 of Example 1 to obtain tert-butyl (4-(2-(4-(4-((2,6-dioxopiperidin-3-yl)amino)-2-fluorophenyl)piperazin-1-yl)ethyl)piperidin-1-yl)carbamate (0.2 g, 360.46 μmol, 56.17% yield, 96% purity) as a blue solid. MS(M+H)⁺=533.4

### Step 3. Synthesis of 3-((4-(4-(2-(1-aminopiperidin-4-yl)ethyl)piperazin-1-yl)-3-fluorophenyl)amino)piperidine-2,6-dione (5)

It was synthesized by similar method described in step 6 of Example 3 to obtain 3-((4-(4-(2-(1-aminopiperidin-4-yl)ethyl)piperazin-1-yl)-3-fluorophenyl)amino)piperidine-2,6-dione (150 mg, 274.45 mol, 97.46% yield, TFA) as yellow oil. MS(M+H)⁺=433.4

### Step 4. Synthesis of N(4-(2-(4-(4-((2,6-dioxopiperidin-3-yl)amino)-2-fluorophenyl)piperazin-1-yl)ethyl)piperidin-1-yl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (Compound 28)

It was synthesized by similar method described in step 10 of Example 2 to obtain N(4-(2-(4-(4-((2,6-dioxopiperidin-3-yl)amino)-2-fluorophenyl)piperazin-1-yl)ethyl)piperidin-1-yl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (105.3 mg, 110.26 μmol, 40.18% yield, 98% purity, TFA) as a white solid. MS(M+H)⁺=822.4.

¹H NMR (400 MHz, DMSO-d₆) δ=10.80 (s, 1H), 9.90-9.61 (m, 1H), 9.58-9.37 (m, 1H), 8.39-8.15 (m, 3H), 7.56-7.38 (m, 2H), 6.90 (t, J=9.3 Hz, 1H), 6.56 (dd, J=2.1, 14.9 Hz, 1H), 6.49-6.42 (m, 1H), 4.29-4.18 (m, 3H), 3.95 (s, 3H), 3.69-2.61 (m, 2H), 3.61-3.54 (m, 2H), 3.33 (s, 3H), 3.34-3.26 (m, 2H), 3.25-3.11 (m, 6H), 3.00-2.91 (m, 2H), 2.92-2.85 (m, 2H), 2.79-2.69 (m, 1H), 2.67-2.61 (m, 1H), 2.12-2.04 (m, 1H), 1.94-1.83 (m, 1H), 1.81-1.74 (m, 2H), 1.70-1.61 (m, 2H), 1.45-1.36 (m, 3H), 1.22 (t, J=7.0 Hz, 3H).

### Example 29. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro phenyl)piperazin-1-yl)ethyl)piperidin-1-yl)-3-methoxybenzamide (Compound 29)

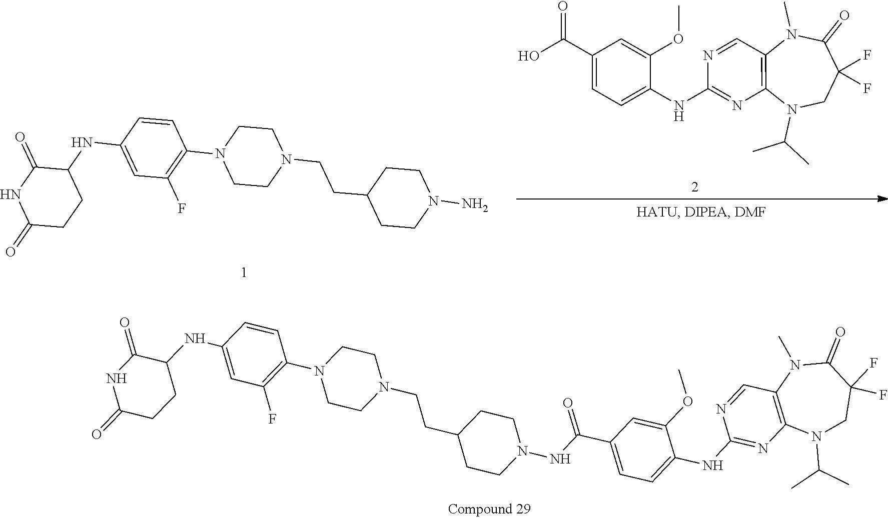 [SMILES: NN1CCC(CCN2CCN(c3ccc(NC4CCC(=O)NC4=O)cc3F)CC2)CC1 ; COc1cc(C(=O)O)ccc1Nc1ncc2c(n1)N(C(C)C)CC(F)(F)C(=O)N2C ; COc1cc(C(=O)NN2CCC(CCN3CCN(c4ccc(NC5CCC(=O)NC5=O)cc4F)CC3)CC2)ccc1Nc1ncc2c(n1)N(C(C)C)CC(F)(F)C(=O)N2C]

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluorophenyl)piperazin-1-yl)ethyl)piperidin-1-yl)-3-methoxybenzamide (82 mg, 67.52 mol, 31.62% yield, 97% purity, 3TFA salt) as a white solid. MS(M+H)⁺=836.3.

¹H NMR (400 MHz, DMSO-d₆) δ=10.79 (s, 1H), 9.81-9.61 (m, 1H), 9.58-9.34 (m, 1H), 8.35-8.12 (m, 3H), 7.54-7.36 (m, 2H), 6.90 (t, J=9.4 Hz, 1H), 6.55 (dd, J=2.3, 14.9 Hz, 1H), 6.50-6.38 (m, 1H), 4.94-4.83 (m, 1H), 4.28 (br dd, J=4.9, 11.6 Hz, 1H), 4.08 (br s, 2H), 3.94 (s, 3H), 3.57 (br d, J=11.5 Hz, 2H), 3.32 (s, 3H), 3.28 (br d, J=11.4 Hz, 2H), 3.20 (br d, J=8.8 Hz, 3H), 3.17-3.10 (m, 3H), 3.00-2.91 (m, 2H), 2.90-2.79 (m, 2H), 2.78-2.67 (m, 1H), 2.63-2.54 (m, 1H), 2.13-2.02 (m, 1H), 1.93-1.82 (m, 1H), 1.81-1.71 (m, 2H), 1.70-1.55 (m, 2H), 1.49-1.30 (m, 3H), 1.24 (d, J=6.7 Hz, 6H).

### Example 30. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2-fluorophenyl)piperidin-4-yl)ethyl)piperidin-4-yl)-3-methoxybenzamide (Compound 30)

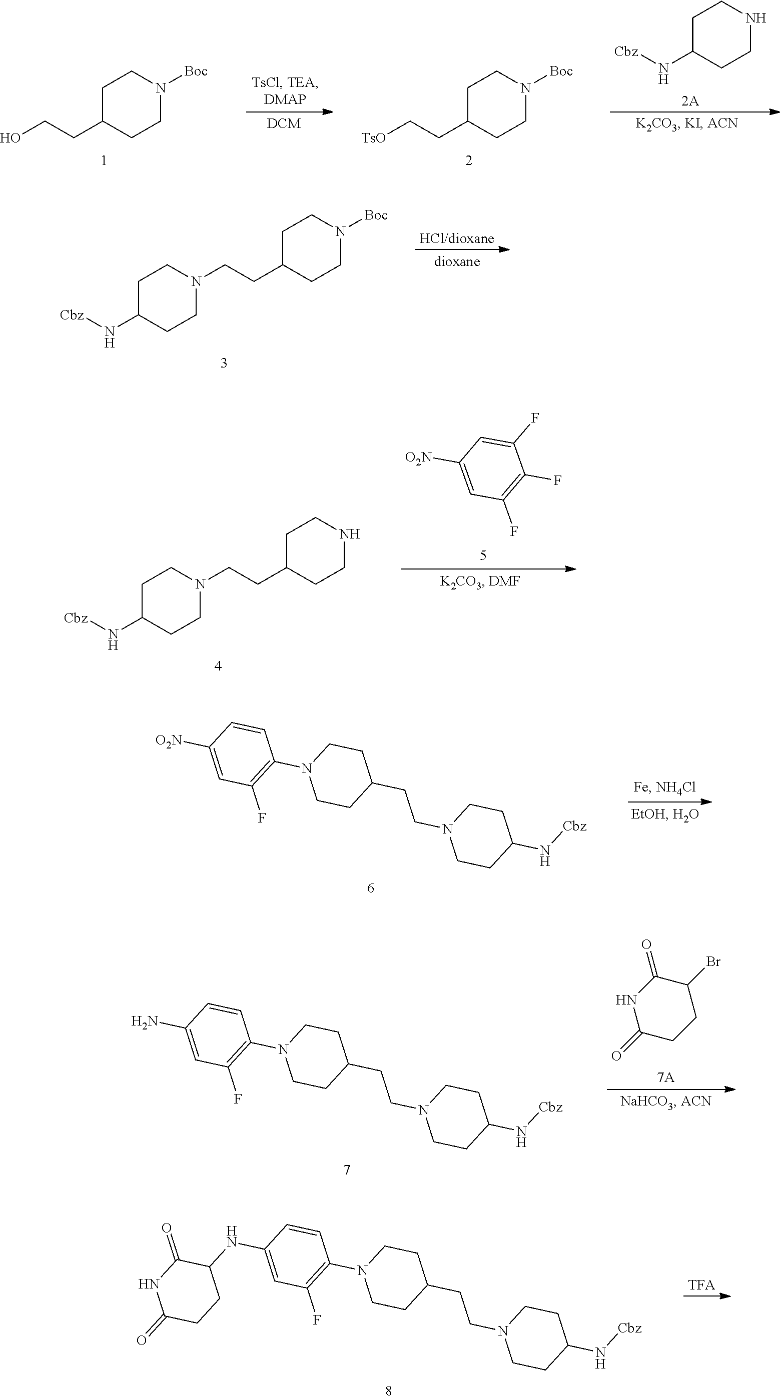

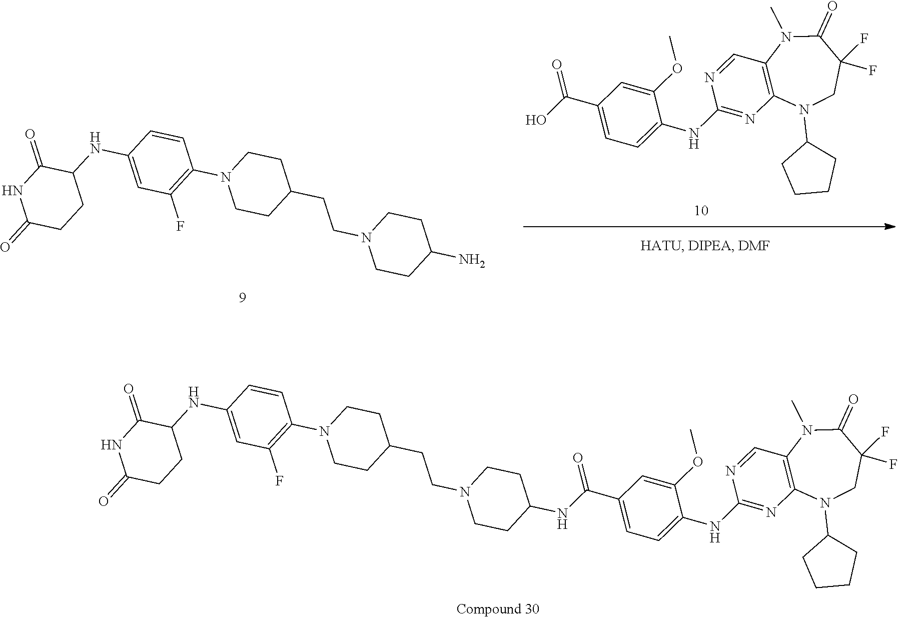 [SMILES: NC1CCN(CCC2CCN(c3ccc(NC4CCC(=O)NC4=O)cc3F)CC2)CC1 ; COc1cc(C(=O)O)ccc1Nc1ncc2c(n1)N(C1CCCC1)CC(F)(F)C(=O)N2C ; COc1cc(C(=O)NC2CCN(CCC3CCN(c4ccc(NC5CCC(=O)NC5=O)cc4F)CC3)CC2)ccc1Nc1ncc2c(n1)N(C1CCCC1)CC(F)(F)C(=O)N2C]

### Step 1. Synthesis of tert-butyl 4-(2-(tosyloxy)ethyl)piperidine-1-carboxylate (2)

It was synthesized by similar method described in step 7 of Example 25, addition DMAP (26.64 mg, 218.04 μmol) to obtain tert-butyl 4-(2-(tosyloxy)ethyl)piperidine-1-carboxylate (8.56 g, 19.64 mmol, 90.08% yield, 88% purity) as a light yellow oil. MS(M−100+H)⁺=284.1.

### Step 2. Synthesis of tert-butyl 4-(2-(4-(((benzyloxy)carbonyl)amino)piperidin-1-yl)ethyl)piperidine-1-carboxylate (3)

To a solution of tert-butyl 4-(2-(tosyloxy)ethyl)piperidine-1-carboxylate (5 g, 13.04 mmol) and benzyl piperidin-4-ylcarbamate (3.1 g, 13.23 mmol) in ACN (80 mL) were added K₂CO₃ (3.60 g, 26.08 mmol) and KI (43.29 mg, 260.76 mol), the mixture was stirred at 60° C. for 16 hours under N₂ atmosphere. LCMS showed tert-butyl 4-(2-(tosyloxy)ethyl)piperidine-1-carboxylate was consumed completely and 98% of desired mass. The reaction mixture was filtered and the filter cake was washed with EtOAc (50 mL), the filtrate was concentrated in vacuum. The residue was purified by flash silica gel chromatography (20 g SepaFlash® Silica Flash Column, Eluent of 25-100% EtOAc/Petroleum ether to 10% MeOH/EtOAc gradient @ 200 mL/min) to afford tert-butyl 4-(2-(4-(((benzyloxy)carbonyl)amino)piperidin-1-yl)ethyl)piperidine-1-carboxylate (5.53 g, 12.41 mmol, 95.19% yield) as a light brown solid. MS(M+H)⁺=446.4.

### Step 3. Synthesis of benzyl (1-(2-(piperidin-4-yl)ethyl)piperidin-4-yl)carbamate (4)

It was synthesized by similar method described in step 5 of Example 1 to obtain benzyl (1-(2-(piperidin-4-yl)ethyl)piperidin-4-yl)carbamate (6.7 g, HCl salt) as a light brown solid which was used directly. MS(M+H)⁺=346.2.

### Step 4. Synthesis of benzyl (1-(2-(1-(2-fluoro-4-nitrophenyl)piperidin-4-yl)ethyl)piperidin-4-yl)carbamate (6)

A mixture of benzyl (1-(2-(piperidin-4-yl)ethyl)piperidin-4-yl)carbamate (6.7 g, 17.54 mmol, HCl salt), 1,2-difluoro-4-nitrobenzene (5.58 g, 35.08 mmol, 3.88 mL) and K₂CO₃ (7.27 g, 52.63 mmol) in DMF (70 mL) was stirred at 20° C. for 16 hours. LCMS showed benzyl (1-(2-(piperidin-4-yl)ethyl)piperidin-4-yl)carbamate was consumed completely and 39% of desired mass. The mixture was diluted with H₂O (600 mL) and extracted with a mixture (EtOAc:MeOH=10:1, 200 mL×3). The combined organic layers were washed with brine (300 mL×5), dried over MgSO₄, filtered and concentrated in vacuum. The residue was triturated with a mixture solution (Petroleum ether/EtOAc=5/1, 40 mL) for 10 minutes, the suspension was filtered and the filter cake was washed with a mixture solution (Petroleum ether/EtOAc=5/1, 40 mL). The filter cake was collected and dried in vacuum to afford benzyl (1-(2-(1-(2-fluoro-4-nitrophenyl)piperidin-4-yl)ethyl)piperidin-4-yl)carbamate (5.33 g, 11.00 mmol, 62.70% yield) as a yellow solid. MS(M+H)⁺=485.2.

### Step 5. Synthesis of benzyl (1-(2-(1-(4-amino-2-fluorophenyl)piperidin-4-yl)ethyl)piperidin-4-yl)carbamate (7)

It was synthesized by similar method described in step 4 of Example 22 to obtain benzyl (1-(2-(1-(4-amino-2-fluorophenyl)piperidin-4-yl)ethyl)piperidin-4-yl)carbamate (4.9 g, 10.78 mmol, 98.55% yield) as a red solid. MS(M+H)⁺=455.2.

### Step 6. Synthesis of benzyl (1-(2-(1-(4-((2,6-dioxopiperidin-3-yl)amino)-2-fluorophenyl)piperidin-4-yl)ethyl)piperidin-4-yl)carbamate (8)

It was synthesized by similar method described in step 5 of Example 22 to obtain benzyl (1-(2-(1-(4-((2,6-dioxopiperidin-3-yl)amino)-2-fluorophenyl)piperidin-4-yl)ethyl)piperidin-4-yl)carbamate (480 mg, 610.95 mol, 34.72% yield, 72% purity) as a light gray solid. MS(M+H)⁺=566.5.

### Step 7. Synthesis of 3-((4-(4-(2-(4-aminopiperidin-1-yl)ethyl)piperidin-1-yl)-3-fluorophenyl)amino)piperidine-2,6-dione (9)

It was synthesized by similar method described in step 2 of Example 4 to obtain 3-((4-(4-(2-(4-aminopiperidin-1-yl)ethyl)piperidin-1-yl)-3-fluorophenyl)amino)piperidine-2,6-dione (195 mg, TFA salt) as a green oil, which was used directly. MS(M+H)⁺=432.2.

### Step 8. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2-fluorophenyl)piperidin-4-yl)ethyl)piperidin-4-yl)-3-methoxybenzamide (Compound 30)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2-fluorophen yl)piperidin-4-yl)ethyl)piperidin-4-yl)-3-methoxybenzamide (105.7 mg, 85.23 mol, 23.83% yield, 97% purity, 3TFA salt) as a light gray solid. MS(M+H)⁺=861.3.

¹H NMR (400 MHz, DMSO-d₆) δ=10.82 (s, 1H), 9.59-9.32 (m, 1H), 8.47-8.35 (m, 1H), 8.33-8.25 (m, 2H), 8.23 (d, J=8.7 Hz, 1H), 7.60-7.44 (m, 2H), 7.23-7.01 (m, 1H), 6.68-6.55 (m, 1H), 6.50 (d, J=8.1 Hz, 1H), 4.82-4.74 (m, 1H), 4.38-4.25 (m, 1H), 4.22-3.98 (m, 3H), 3.97-3.84 (m, 3H), 3.58 (d, J=11.1 Hz, 1H), 3.45-1.13 (m, 5H), 3.20-2.87 (m, 4H), 2.07-1.99 (m, 4H), 1.99-1.76 (m, 8H), 1.75-1.62 (m, 6H), 1.61-1.40 (m, 8H).

### Example 31. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-(2-(1-(4-((2,6-dioxopiperidin-3-yl)amino)-2-fluoro phenyl)piperidin-4-yl)ethyl)-7-azaspiro[3.5]nonan-2-yl)-3-methoxybenzamide (Compound 31)

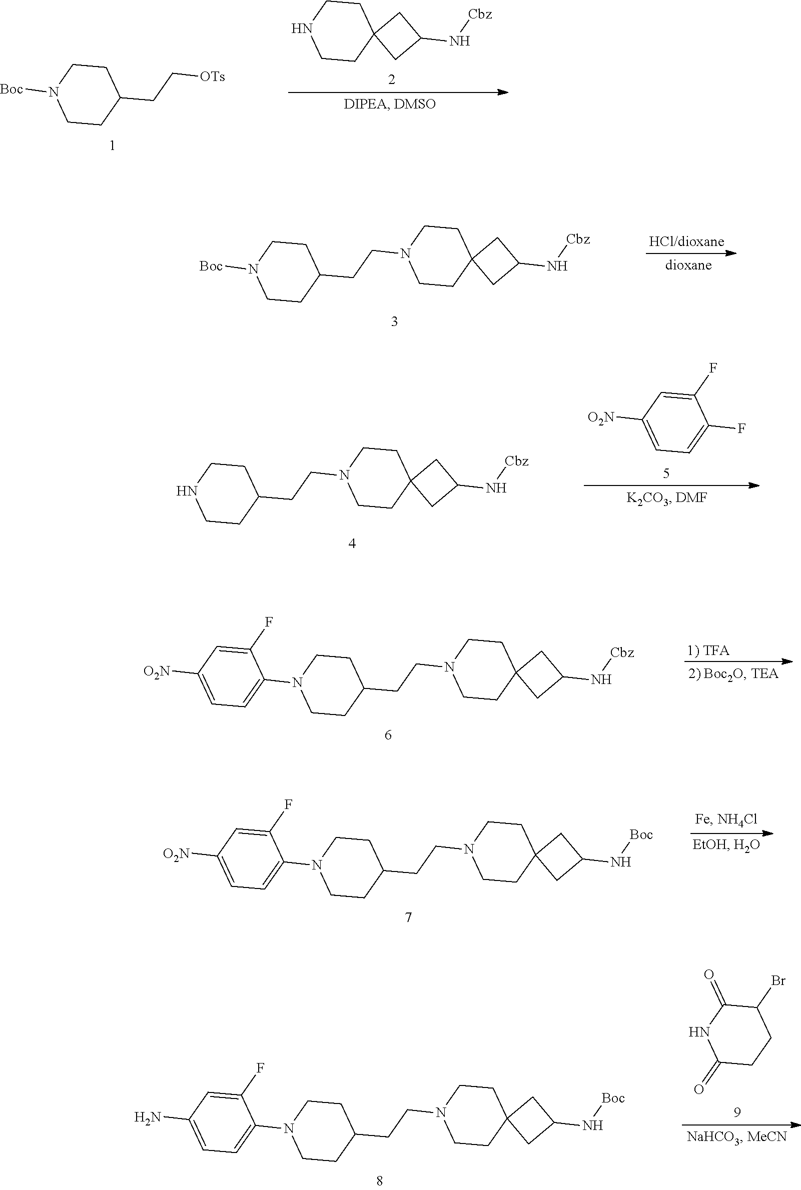

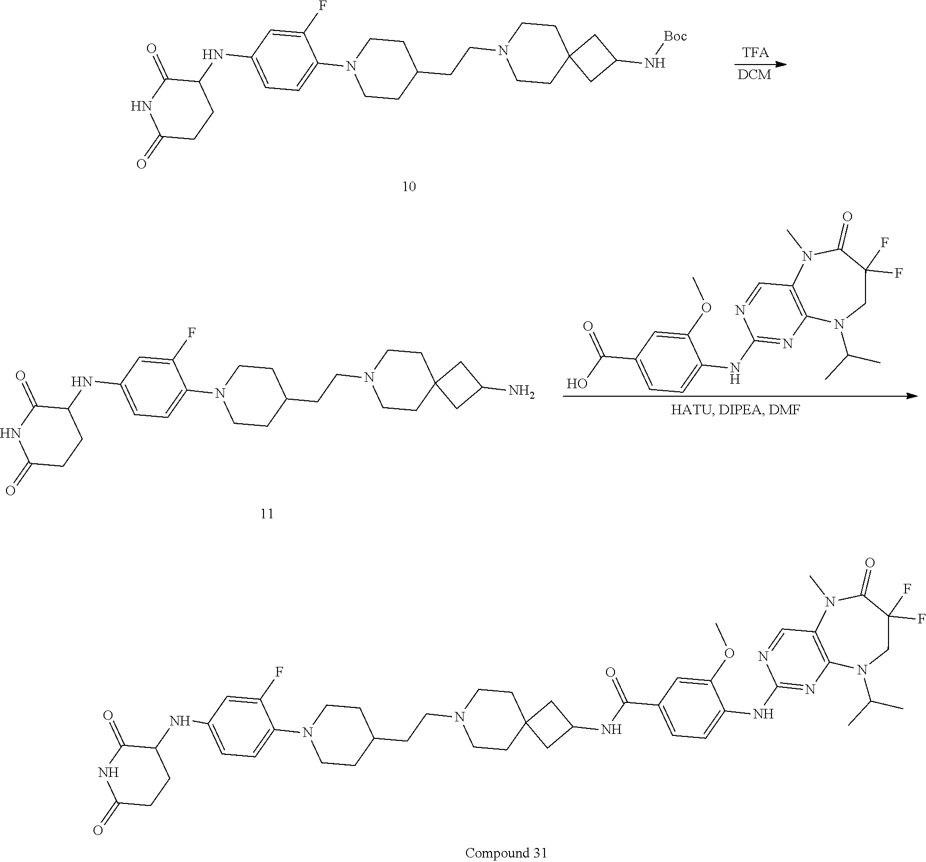 [SMILES: CC(C)(C)OC(=O)NC1CC2(CCN(CCC3CCN(c4ccc(NC5CCC(=O)NC5=O)cc4F)CC3)CC2)C1 ; NC1CC2(CCN(CCC3CCN(c4ccc(NC5CCC(=O)NC5=O)cc4F)CC3)CC2)C1 ; COc1cc(C(=O)O)ccc1Nc1ncc2c(n1)N(C(C)C)CC(F)(F)C(=O)N2C ; COc1cc(C(=O)NC2CC3(CCN(CCC4CCN(c5ccc(NC6CCC(=O)NC6=O)cc5F)CC4)CC3)C2)ccc1Nc1ncc2c(n1)N(C(C)C)CC(F)(F)C(=O)N2C]

### Step 1. Synthesis of tert-butyl 4-(2-(2-(((benzyloxy)carbonyl)amino)-7-azaspiro[3.5]nonan-7-yl)ethyl)piperidine-1-carboxylate (3)

To the solution of benzyl (7-azaspiro[3.5]nonan-2-yl)carbamate (2 g, 6.43 mmol, HCl) and tert-butyl 4-(2-(tosyloxy)ethyl)piperidine-1-carboxylate (3 g, 7.82 mmol) in DMSO (50 mL) was added DIPEA (2.49 g, 19.30 mmol, 3.36 mL) and the resulting mixture was stirred at 80° C. for 16 h. LCMS showed a peak (56%) with desired mass. The resulting mixture was poured into water (200 mL) and extracted with EtOAc (80 mL×3), the combined organic layers were washed with saturated brine (200 mL), dried over anhydrous Na₂SO₄, filtered and concentrated under reduced pressure. The residue was purified by flash silica gel chromatography (50 g SepaFlash® Silica Flash Column, Eluent of 0-100% EtOAc/Petroleum ether gradient @ 100 mL/min) to afford tert-butyl 4-(2-(2-(((benzyloxy)carbonyl)amino)-7-azaspiro[3.5]nonan-7-yl)ethyl)piperidine-1-carboxylate (2 g, 4.12 mmol, 64.00% yield, 100% purity) as yellow oil. MS(M+H)⁺=486.3

### Step 2. Synthesis of benzyl (7-(2-(piperidin-4-yl)ethyl)-7-azaspiro[3.5]nonan-2-yl)carbamate (4)

It was synthesized by similar method described in step 5 of Example 1 to obtain benzyl (7-(2-(piperidin-4-yl)ethyl)-7-azaspiro[3.5]nonan-2-yl)carbamate (2.0 g, crude, HCl) as a yellow solid. MS(M+H)⁺=386.3

### Step 3. Synthesis of benzyl (7-(2-(1-(2-fluoro-4-nitrophenyl)piperidin-4-yl)ethyl)-7-azaspiro[3.5]nonan-2-yl)carbamate (6)

It was synthesized by similar method described in step 4 of Example 30 to obtain benzyl (7-(2-(1-(2-fluoro-4-nitrophenyl)piperidin-4-yl)ethyl)-7-azaspiro[3.5]nonan-2-yl)carba mate (1.2 g, 2.24 mmol, 63.06% yield, 98% purity) as yellow oil. MS(M+H)⁺=525.4

### Step 4. Synthesis of tert-butyl (7-(2-(1-(2-fluoro-4-nitrophenyl)piperidin-4-yl)ethyl)-7-azaspiro[3.5]nonan-2-yl)carbamate (7)

A mixture of benzyl (7-(2-(1-(2-fluoro-4-nitrophenyl)piperidin-4-yl)ethyl)-7-azaspiro[3.5]nonan-2-yl)carba mate (1.2 g, 2.29 mmol) in TFA (18.42 g, 161.55 mmol, 12.00 mL) was stirred at 40° C. for 2 h. The mixture was concentrated, the residue was diluted with THF (20 mL), then Boc₂O (748.81 mg, 3.43 mmol, 788.22 μL) and TEA (694.36 mg, 6.86 mmol) were added, the resulting mixture was stirred at 20° C. for 2 h. LCMS showed a peak (47%) with desired mass. The mixture was diluted with H₂O (50 mL) and extracted with EtOAc (30 mL×3). The combined organic layers were dried over MgSO₄, filtered and concentrated in vacuum. The residue was purified by flash silica gel chromatography (20 g SepaFlash® Silica Flash Column, Eluent of 0-100% EtOAc/Petroleum ether gradient @ 80 mL/min) to afford tert-butyl (7-(2-(1-(2-fluoro-4-nitrophenyl)piperidin-4-yl)ethyl)-7-azaspiro[3.5]nonan-2-yl)carba mate (1.2 g, 2.20 mmol, 96.24% yield, 90% purity) as yellow oil. MS(M+H)⁺=491.2

### Step 5. Synthesis of tert-butyl (7-(2-(1-(4-amino-2-fluorophenyl)piperidin-4-yl)ethyl)-7-azaspiro[3.5]nonan-2-yl) carbamate (8)

It was synthesized by similar method described in step 4 of Example 22 to obtain tert-butyl (7-(2-(1-(4-amino-2-fluorophenyl)piperidin-4-yl)ethyl)-7-azaspiro[3.5]nonan-2-yl)carbamate (0.82 g, 1.48 mmol, 60.41% yield, 83% purity) as yellow oil. MS(M+H)⁺=461.4

### Step 6. Synthesis of tert-butyl (7-(2-(1-(4-((2,6-dioxopiperidin-3-yl)amino)-2-fluorophenyl)piperidin-4-yl)ethyl)-7-azaspiro[3.5]nonan-2-yl)carbamate (10)

It was synthesized by similar method described in step 5 of Example 22 to obtain tert-butyl (7-(2-(1-(4-((2,6-dioxopiperidin-3-yl)amino)-2-fluorophenyl)piperidin-4-yl)ethyl)-7-azaspiro[3.5]nonan-2-yl)carbamate (1.5 g, crude) as a brown solid. MS(M+H)⁺=572.5

### Step 7. Synthesis of 3-((4-(4-(2-(2-amino-7-azaspiro[3.5]nonan-7-yl)ethyl)piperidin-1-yl)-3-fluorophen yl)amino)piperidine-2,6-dione (11)

It was synthesized by similar method described in step 6 of Example 3 to obtain 3-((4-(4-(2-(2-amino-7-azaspiro[3.5]nonan-7-yl)ethyl)piperidin-1-yl)-3-fluorophenyl)amino)piperidine-2,6-dione (1 g, crude, TFA) as black oil. MS(M+H)⁺=472.3

### Step 8. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-(2-(1-(4-((2,6-dioxopiperidin-3-yl)amino)-2-fluoro phenyl)piperidin-4-yl)ethyl)-7-azaspiro[3.5]nonan-2-yl)-3-methoxybenzamide (Compound 31)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(7-(2-(1-(4-((2,6-dioxopiperidin-3-yl)amino)-2-fluorophenyl)piperidin-4-yl)ethyl)-7-azaspiro[3.5]nonan-2-yl)-3-methoxybenzamide (440.4 mg, 440.84 μmol, 25.82% yield, 99% purity, TFA) as a white solid. MS(M+H)⁺=875.4.

¹H NMR (400 MHz, DMSO-d₆) δ=10.81 (s, 1H), 9.46-9.19 (m, 1H), 8.54 (d, J=6.9 Hz, 1H), 8.50-8.37 (m, 1H), 8.25 (s, 1H), 8.22 (d, J=8.3 Hz, 1H), 7.57-7.49 (m, 2H), 7.22-7.07 (m, 1H), 6.66-6.56 (m, 1H), 6.52 (d, J=8.0 Hz, 1H), 4.96-4.84 (m, 1H), 4.51-4.39 (m, 1H), 4.37-4.30 (m, 1H), 4.11 (t, J=13.3 Hz, 2H), 4.03-3.85 (m, 4H), 3.49-3.32 (m, 6H), 3.19-2.86 (m, 5H), 2.85-2.59 (m, 3H), 2.43-2.34 (m, 1H), 2.24-1.93 (m, 5H), 1.92-1.75 (m, 6H), 1.69-1.60 (m, 2H), 1.58-1.49 (m, 3H), 1.25 (d, J=6.6 Hz, 6H).

### Example 32. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-3-fluorophenyl)piperazin-1-yl)ethyl)piperidin-4-yl)-3-methoxybenzamide (Compound 32)

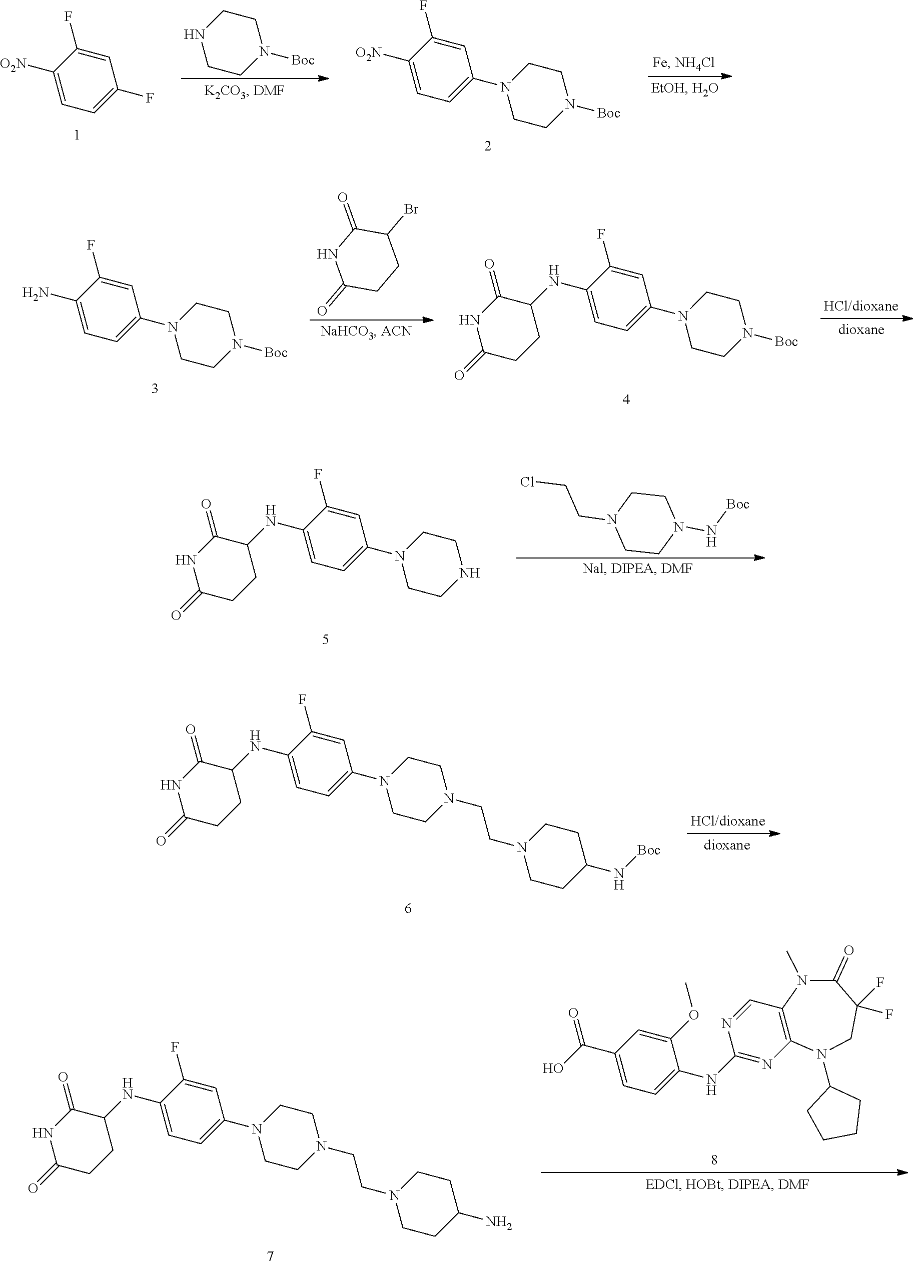

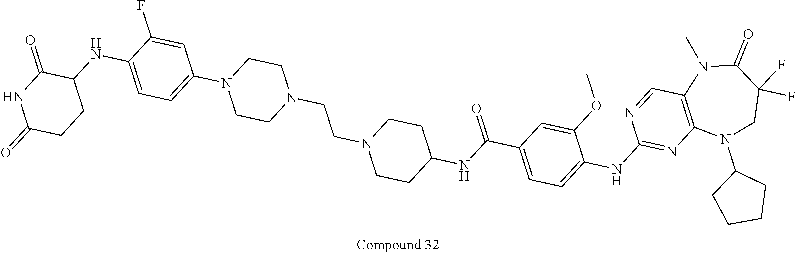 [SMILES: COc1cc(C(=O)NC2CCN(CCN3CCN(c4ccc(NC5CCC(=O)NC5=O)c(F)c4)CC3)CC2)ccc1Nc1ncc2c(n1)N(C1CCCC1)CC(F)(F)C(=O)N2C]

### Step 1. Synthesis of tert-butyl 4-(3-fluoro-4-nitrophenyl)piperazine-1-carboxylate (2)

It was synthesized by similar method described in step 4 of Example 30 to obtain tert-butyl 4-(3-fluoro-4-nitrophenyl)piperazine-1-carboxylate (21 g, 64.55 mmol, 30.06% yield) as a yellow solid. MS(M+H)⁺=325.9.

¹H NMR (400 MHz, CDCl₃) δ=8.02 (t, J=9.1 Hz, 1H), 6.69-6.42 (m, 2H), 3.70-3.54 (m, 4H), 3.48-3.33 (m, 4H), 1.50 (s, 9H)

### Step 2. Synthesis of tert-butyl 4-(4-amino-3-fluorophenyl)piperazine-1-carboxylate (3)

It was synthesized by similar method described in step 4 of Example 22 to obtain tert-butyl 4-(4-amino-3-fluorophenyl)piperazine-1-carboxylate (19 g, crude) as a brown solid. MS(M+H)⁺=296.1.

### Step 3. Synthesis of tert-butyl 4-(4-((2,6-dioxopiperidin-3-yl)amino)-3-fluorophenyl)piperazine-1-carboxylate (4)

It was synthesized by similar method described in step 5 of Example 22 to obtain tert-butyl 4-(4-((2,6-dioxopiperidin-3-yl)amino)-3-fluorophenyl)piperazine-1-carboxylate (25 g, 61.51 mmol, 95.61% yield) as a green solid. MS(M+H)⁺=407.1.

¹H NMR (400 MHz, CDCl₃) δ=8.45 (s, 1H), 6.75-6.57 (m, 3H), 4.59-4.45 (m, 1H), 4.06-3.97 (m, 1H), 3.62-3.51 (m, 4H), 3.04-2.92 (m, 4H), 2.88-2.79 (m, 1H), 2.78-2.66 (m, 1H), 2.51-2.43 (m, 1H), 2.00-1.88 (m, 1H), 1.48 (s, 9H)

### Step 4. Synthesis of 3-((2-fluoro-4-(piperazin-1-yl)phenyl)amino)piperidine-2,6-dione (5)

It was synthesized by similar method described in step 5 of Example 1 to obtain 3-((2-fluoro-4-(piperazin-1-yl)phenyl)amino)piperidine-2,6-dione (21 g, crude, HCl) as a green solid. MS(M+H)⁺=306.9.

### Step 5. Synthesis of tert-butyl (1-(2-(4-(4-((2,6-dioxopiperidin-3-yl)amino)-3-fluorophenyl)piperazin-1-yl)ethyl)piperidin-4-yl)carbamate (6)

It was synthesized by similar method described in step 3 of Example 23 to obtain tert-butyl (1-(2-(4-(4-((2,6-dioxopiperidin-3-yl)amino)-3-fluorophenyl)piperazin-1-yl)ethyl)piperidin-4-yl)carbamate (2.9 g, 5.44 mmol, 37.33% yield) as a brown solid. MS(M+H)⁺=533.3.

### Step 6. Synthesis of 3-((4-(4-(2-(4-aminopiperidin-1-yl)ethyl)piperazin-1-yl)-2-fluorophenyl)amino)piperidine-2,6-dione (7)

It was synthesized by similar method described in step 4 to obtain 3-((4-(4-(2-(4-aminopiperidin-1-yl)ethyl)piperazin-1-yl)-2-fluorophenyl)amino)piperidine-2,6-dione (2.4 g, crude, HCl) as a green solid. MS(M+H)⁺=433.2.

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-3-fluorophenyl)piperazin-1-yl)ethyl)piperidin-4-yl)-3-methoxybenzamide (Compound 32)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-3-fluorophen yl)piperazin-1-yl)ethyl)piperidin-4-yl)-3-methoxybenzamide (2.43 g, 2.75 mmol, 61.43% yield, 97.4% purity) as an off-white solid. MS(M+H)⁺=862.5.

¹H NMR (400 MHz, DMSO-d₆) δ=10.81 (s, 1H), 8.34-8.24 (m, 2H), 8.12 (d, J=7.8 Hz, 1H), 7.96 (s, 1H), 7.55-7.46 (m, 2H), 6.81-6.69 (m, 2H), 6.63-6.55 (m, 1H), 5.12-5.02 (m, 1H), 4.82-4.71 (m, 1H), 4.32-4.21 (m, 1H), 4.05 (t, J=14.1 Hz, 2H), 3.94 (s, 3H), 3.84-3.71 (m, 1H), 3.37-3.34 (m, 2H), 3.04-2.85 (m, 6H), 2.80-2.70 (m, 1H), 2.62-2.45 (m, 10H), 2.14-1.88 (m, 6H), 1.84-1.67 (m, 4H), 1.67-1.52 (m, 6H).

### Example 33. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-3-fluoro phenyl)piperazin-1-yl)ethyl)piperidin-1-yl)-3-methoxybenzamide (Compound 33)

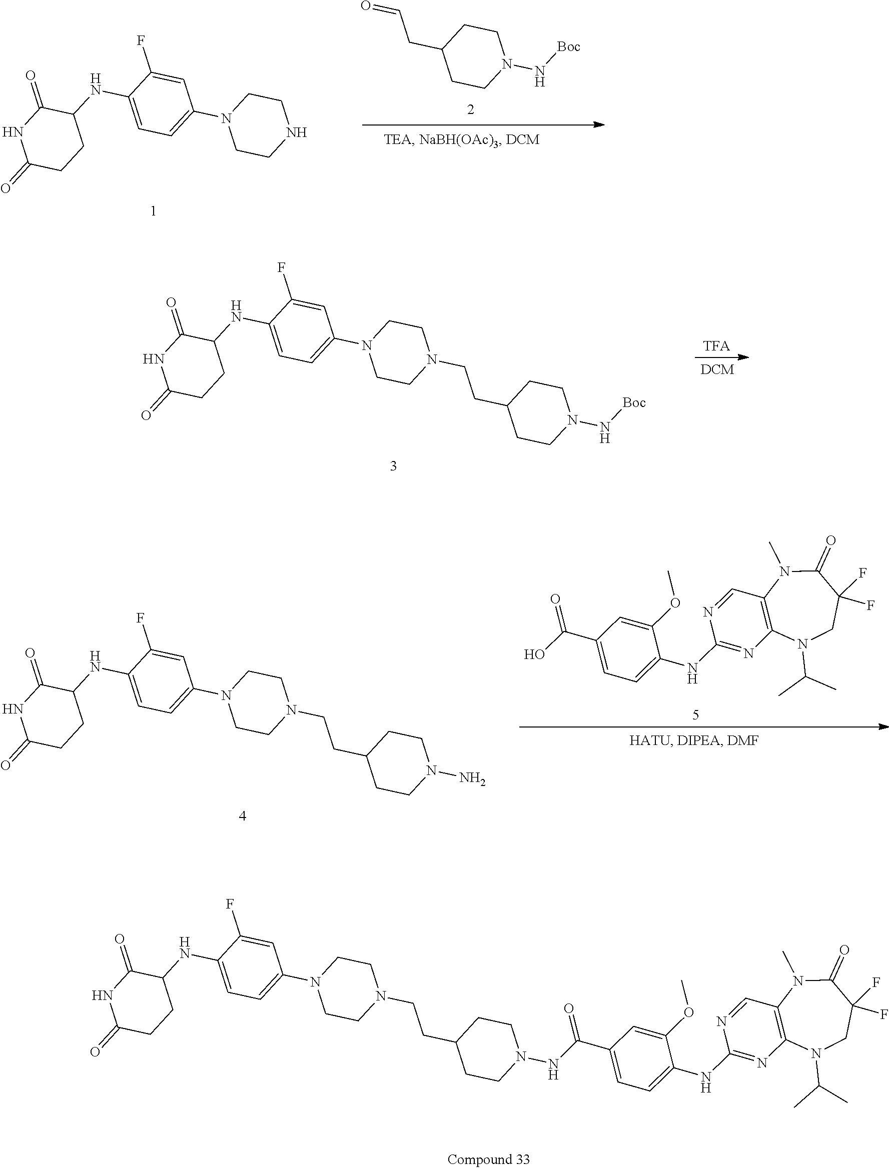 [SMILES: O=C1CCC(Nc2ccc(N3CCNCC3)cc2F)C(=O)N1 ; CC(C)(C)OC(=O)NN1CCC(CC=O)CC1 ; CC(C)(C)OC(=O)NN1CCC(CCN2CCN(c3ccc(NC4CCC(=O)NC4=O)c(F)c3)CC2)CC1 ; NN1CCC(CCN2CCN(c3ccc(NC4CCC(=O)NC4=O)c(F)c3)CC2)CC1 ; COc1cc(C(=O)O)ccc1Nc1ncc2c(n1)N(C(C)C)CC(F)(F)C(=O)N2C ; COc1cc(C(=O)NN2CCC(CCN3CCN(c4ccc(NC5CCC(=O)NC5=O)c(F)c4)CC3)CC2)ccc1Nc1ncc2c(n1)N(C(C)C)CC(F)(F)C(=O)N2C]

### Step 1. Synthesis of tert-butyl (4-(2-(4-(4-((2,6-dioxopiperidin-3-yl)amino)-3-fluorophenyl)piperazin-1-yl)ethyl)piperidin-1-yl)carbamate (3)

It was synthesized by similar method described in step 6 of Example 1 to obtain tert-butyl (4-(2-(4-(4-((2,6-dioxopiperidin-3-yl)amino)-3-fluorophenyl)piperazin-1-yl)ethyl)piperidin-1-yl)carbamate (7.8 g, 14.64 mmol, 55.78% yield) as a gray solid. MS(M+H)⁺=533.3.

### Step 2. Synthesis of 3-((4-(4-(2-(1-aminopiperidin-4-yl)ethyl)piperazin-1-yl)-2-fluorophenyl)amino)piperidine-2,6-dione (4)

It was synthesized by similar method described in step 6 of Example 3 to obtain 3-((4-(4-(2-(1-aminopiperidin-4-yl)ethyl)piperazin-1-yl)-2-fluorophenyl)amino)piperidine-2,6-dione (2.1 g, crude, TFA) as a green oil. MS(M+H)⁺=433.2.

### Step 3. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-3-fluoro phenyl)piperazin-1-yl)ethyl)piperidin-1-yl)-3-methoxybenzamide (Compound 33)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-3-fluorophenyl)piperazin-1-yl)ethyl)piperidin-1-yl)-3-methoxybenzamide (1.30 g, 1.52 mmol, 49.40% yield, 98.0% purity) as an off-white solid. MS(M+H)⁺=836.5.

¹H NMR (400 MHz, DMSO-d₆) δ=10.86-10.77 (m, 1H), 9.25 (s, 1H), 8.35-8.28 (m, 1H), 8.22 (s, 1H), 7.91-7.84 (m, 1H), 7.47-7.39 (m, 2H), 6.79-6.69 (m, 2H), 6.62-6.54 (m, 1H), 5.11-5.03 (m, 1H), 4.88 (t, J=13.2 Hz, 1H), 4.32-4.22 (m, 1H), 4.08-4.00 (m, 2H), 3.93 (s, 3H), 3.34-3.31 (m, 4H), 3.04-2.93 (m, 6H), 2.79-2.68 (m, 3H), 2.61-2.51 (m, 2H), 2.47-2.43 (m, 2H), 2.37-2.29 (m, 2H), 2.13-2.06 (m, 1H), 2.00-1.90 (m, 1H), 1.76-1.66 (m, 2H), 1.46-1.38 (m, 2H), 1.34-1.27 (m, 3H), 1.25 (d, J=6.5 Hz, 6H)

### Example 34. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-3-fluorophenyl)piperazin-1-yl)ethyl)piperidin-1-yl)-3-methoxybenzamide (Compound 34)

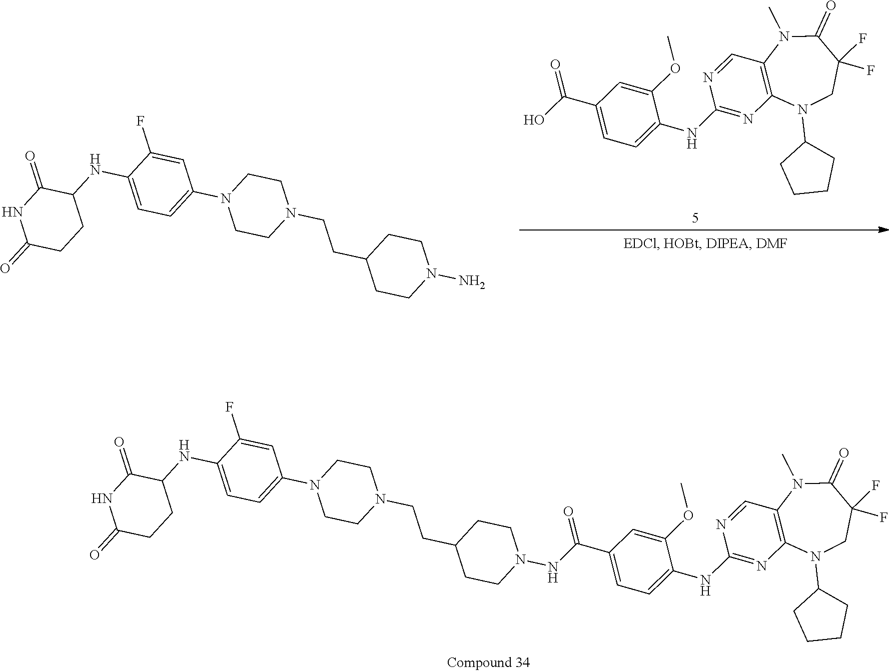 [SMILES: NN1CCC(CCN2CCN(c3ccc(NC4CCC(=O)NC4=O)c(F)c3)CC2)CC1 ; COc1cc(C(=O)O)ccc1Nc1ncc2c(n1)N(C1CCCC1)CC(F)(F)C(=O)N2C ; COc1cc(C(=O)NN2CCC(CCN3CCN(c4ccc(NC5CCC(=O)NC5=O)c(F)c4)CC3)CC2)ccc1Nc1ncc2c(n1)N(C1CCCC1)CC(F)(F)C(=O)N2C]

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (1.2 g, 2.68 mmol) in DMF (16 mL) were added EDCI (960.00 mg, 5.01 mmol), HOBt (660.00 mg, 4.88 mmol), DIPEA (2.23 g, 17.22 mmol, 3.00 mL) and 3-((4-(4-(2-(1-aminopiperidin-4-yl)ethyl)piperazin-1-yl)-2-fluorophenyl)amino)piperidine-2,6-dione (2.30 g, 4.21 mmol, TFA) at 20° C. The mixture was stirred at 20° C. for 16 h under N₂ atmosphere. LCMS showed a peak (48%) with the desired mass. The reaction mixture was diluted with H₂O (30 mL), the mixture was extracted with EtOAc (60 mL×2). The combined organic layers were washed with brine (50 mL×3), dried over Na₂SO₄, filtered and concentrated under reduced pressure. The residue was purified by flash silica gel chromatography (Biotage; 20 g SepaFlash® Silica Flash Column, Eluent of 0~10% MeOH:DCM gradient, 60 mL/min). The product was repurified by prep-HPLC (column: Phenomene x luna C18 150×25 mmx 10 um; mobile phase: [water (TFA)-ACN]; gradient: 21%-51% B over 10 min, Column Temp: 30° C.). The eluent was lyophilized and diluted with HCl (1M, 40 mL), and the mixture was added dropwise to the Na₂CO₃ (1M, 70 mL) at 0° C., and the suspension was filtered and the filter cake was washed with H₂O (80 mL×3) and then lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-3-fluorophen yl)piperazin-1-yl)ethyl)piperidin-1-yl)-3-methoxybenzamide (656.9 mg, 740.01 mol, 27.59% yield, 97.1% purity) as an off-white solid. MS(M+H)⁺=862.5.

¹H NMR (400 MHz, DMSO-d₆) δ=10.86-10.76 (m, 1H), 9.28 (s, 1H), 8.35-8.22 (m, 2H), 7.97 (s, 1H), 7.49-7.35 (m, 2H), 6.81-6.68 (m, 2H), 6.62-6.56 (m, 1H), 5.10-5.03 (m, 1H), 4.82-4.72 (m, 1H), 4.31-4.22 (m, 1H), 4.11-4.00 (m, 2H), 3.93 (s, 3H), 3.37-3.33 (m, 4H), 3.04-2.87 (m, 6H), 2.83-2.69 (m, 3H), 2.62-2.51 (m, 2H), 2.49-2.44 (m, 2H), 2.40-2.30 (m, 2H), 2.13-2.06 (m, 1H), 2.01-1.89 (m, 3H), 1.77-1.66 (m, 4H), 1.65-1.55 (m, 4H), 1.47-1.37 (m, 2H), 1.36-1.23 (m, 3H).

### Example 35. Synthesis of N(4-(2-(4-(4-((2,6-dioxopiperidin-3-yl)amino)-3-fluorophenyl)piperazin-1-yl)ethyl)piperidin-1-yl)-4-((9-ethyl-7,7-difluoro-5-methyl-6-oxo-6,7,8,9-tetrahydro-5H-pyrimido[4,5-b][1,4]diazepin-2-yl)amino)-2-fluoro-5-methoxybenzamide (Compound 35)

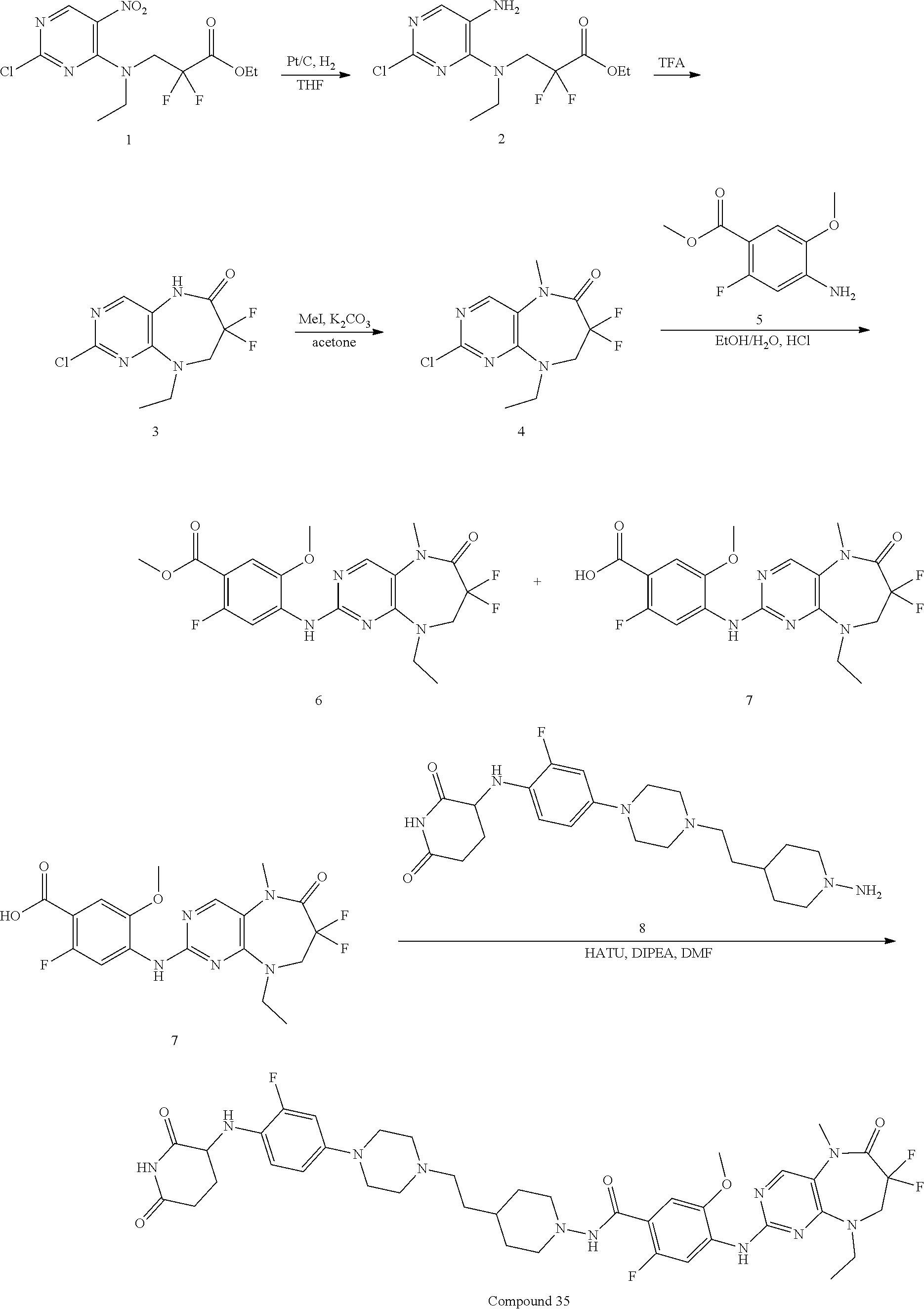

### Step 1. Synthesis of ethyl 3-((5-amino-2-chloropyrimidin-4-yl)(ethyl)amino)-2,2-difluoropropanoate (2)

A solution of ethyl 3-((2-chloro-5-nitropyrimidin-4-yl)(ethyl)amino)-2,2-difluoropropanoate (44.00 g, 111.72 mmol) in THF (900 mL) was stirred at 20° C. until becoming clear solution. Adjust the H₂ back pressure regulator to 1 MPa, the flow rate of H₂ to 95 ml/min, heated the fixed bed Pt/C (30 g, 1% purity) to 50° C. Then the solution was pumped into the reactor at a flow rate of 3 mL/min. After the reaction was finished, washing the tubing with THF (100 mL), collected all the reaction solution. LCMS showed mass. The reaction mixture was filtered and the filtrate was concentrated under reduced pressure to afford ethyl 3-((5-amino-2-chloropyrimidin-4-yl)(ethyl)amino)-2,2-difluoropropanoate (42.00 g, crude) as a pale yellow oil. MS(M+H)⁺=309.1.

### Step 2. Synthesis of 2-chloro-9-ethyl-7,7-difluoro-5,7,8,9-tetrahydro-6H-pyrimido[4,5-b][1,4]diazepin-6-one (3)

A mixture of ethyl 3-((5-amino-2-chloropyrimidin-4-yl)(ethyl)amino)-2,2-difluoropropanoate (42.00 g, 136.05 mmol) in TFA (50 mL) was stirred at 50° C. for 4 hours. LCMS showed a main peak (90%) with desired mass. The mixture was concentrated, the residue was diluted with EtOAc (500 mL), then washed with NaHCO₃ (100 mL×2) and brine (100 mL×2), dried over Na₂SO₄, filtered and concentrated under reduced pressure. The residue was triturated with MTBE (100 mL) at 25° C. for 10 min, filtered. The filter cake was collected and dried to afford 2-chloro-9-ethyl-7,7-difluoro-5,7,8,9-tetrahydro-6H-pyrimido[4,5-b][1,4]diazepin-6-one (26.00 g, 98.00 mmol, 72.04% yield, 99% purity) as a pale yellow solid. MS(M+H)⁺=263.1.

¹H NMR (400 MHz, DMSO-d₆) δ=7.92 (s, 1H), 7.90-7.84 (m, 1H), 3.93 (t, J=10.4 Hz, 2H), 3.89-3.81 (m, 2H), 1.31 (t, J=6.8 Hz, 3H).

### Step 3. Synthesis of 2-chloro-9-ethyl-7,7-difluoro-5-methyl-5,7,8,9-tetrahydro-6H-pyrimido[4,5-b][1,4]diazepin-6-one (4)

It was synthesized by similar method described in step 8 of Example 4 to obtain 2-chloro-9-ethyl-7,7-difluoro-5-methyl-5,7,8,9-tetrahydro-6H-pyrimido[4,5-b][1,4]diazepin-6-one (25.00 g, 85.84 mmol, 86.72% yield, 95% purity) as a white solid. MS(M+H)⁺=277.1.

¹H NMR (400 MHz, DMSO-d₆) δ=8.31 (s, 1H), 4.26 (t, J=12.8 Hz, 2H), 3.69-3.58 (m, 2H), 3.33 (s, 3H), 1.16 (t, J=7.2 Hz, 3H).

### Step 4. Synthesis of methyl 4-((9-ethyl-7,7-difluoro-5-methyl-6-oxo-6,7,8,9-tetrahydro-5H-pyrimido[4,5-b][1,4]diazepin-2-yl)amino)-2-fluoro-5-methoxybenzoate and 4-((9-ethyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (6 & 7)

It was synthesized by similar method described in step 9 of Example 4 to obtain a mixture of 4-((9-ethyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (3.4 g, 5.68 mmol, 19.63% yield, 71% purity) as a pale yellow solid and methyl 4-((9-ethyl-7,7-difluoro-5-methyl-6-oxo-6,7,8,9-tetrahydro-5H-pyrimido[4,5-b][1,4]diazepin-2-yl)amino)-2-fluoro-5-methoxybenzoate (7 g, 10.83 mmol, 37.47% yield, 68% purity) as a pale yellow solid. MS(M+H)⁺=426.2, 440.3.

### Step 5. Synthesis of N(4-(2-(4-(4-((2,6-dioxopiperidin-3-yl)amino)-3-fluorophenyl)piperazin-1-yl)ethyl)piperidin-1-yl)-4-((9-ethyl-7,7-difluoro-5-methyl-6-oxo-6,7,8,9-tetrahydro-5H-pyrimido[4,5-b][1,4]diazepin-2-yl)amino)-2-fluoro-5-methoxybenzamide (Compound 35)

It was synthesized by similar method described in step 10 of Example 2 to obtain N(4-(2-(4-(4-((2,6-dioxopiperidin-3-yl)amino)-3-fluorophenyl)piperazin-1-yl)ethyl)piperidin-1-yl)-4-((9-ethyl-7,7-difluoro-5-methyl-6-oxo-6,7,8,9-tetrahydro-5H-pyrimido[4,5-b][1,4]diazepin-2-yl)amino)-2-fluoro-5-methoxybenzamide (1.40 g, 1.64 mmol, 36.42% yield, 98.1% purity) as a gray solid. MS(M+H)⁺=840.4.

¹H NMR (400 MHz, DMSO-d₆) δ=8.38 (d, J=14.4 Hz, 1H), 8.07 (s, 1H), 7.93 7.86 (m, 1H), 7.81 (s, 1H), 7.56 (d, J=9.2 Hz, 1H), 7.47 (d, J=16.8 Hz, 1H), 6.75 6.60 (m, 3H), 4.53-4.44 (m, 1H), 4.06-3.95 (m, 6H), 3.75-3.66 (m, 2H), 3.42 (s, 3H), 3.33-3.23 (m, 2H), 3.14-3.27 (m, 4H), 2.93-2.83 (m, 1H), 2.80-2.67 (m, 1H), 2.66-2.56 (m, 6H), 2.54-2.41 (m, 3H), 2.05-1.92 (m, 1H), 1.82-1.74 (m, 2H), 1.57-1.45 (m, 4H), 1.43-1.38 (m, 1H), 1.36 (d, J=6.8 Hz, 3H).

### Example 36. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-3-fluoro phenyl)piperidin-4-yl)methyl)-7-azaspiro[3.5]nonan-2-yl)-3-methoxybenzamide (Compound 36)

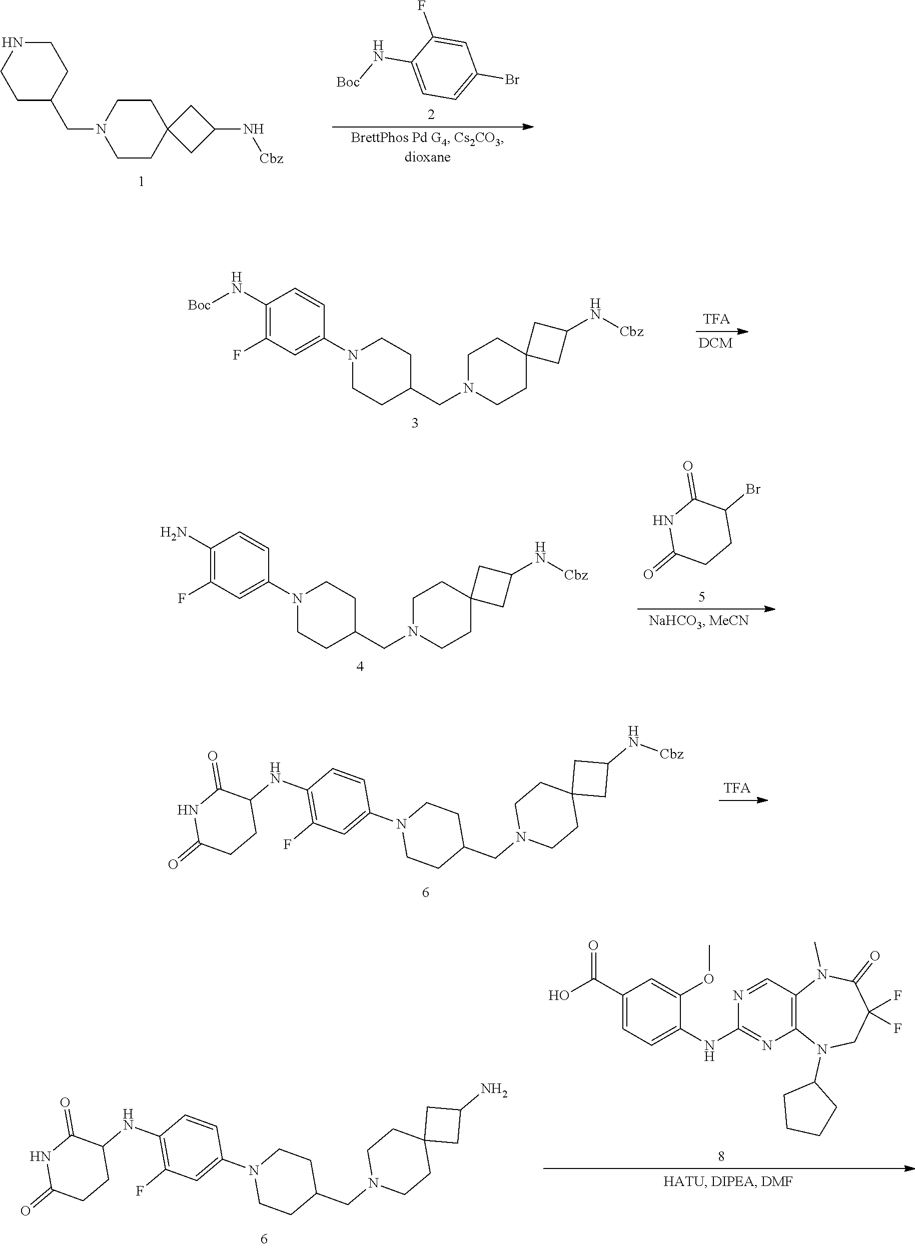

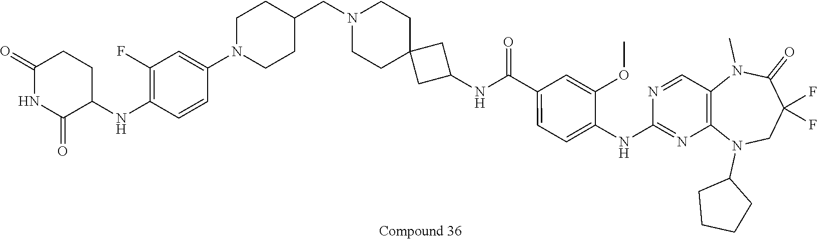 [SMILES: COc1cc(C(=O)NC2CC3(CCN(CC4CCN(c5ccc(NC6CCC(=O)NC6=O)c(F)c5)CC4)CC3)C2)ccc1Nc1ncc2c(n1)N(C1CCCC1)CC(F)(F)C(=O)N2C]

### Step 1. Synthesis of benzyl (7-((1-(4-((tert-butoxycarbonyl)amino)-3-fluorophenyl)piperidin-4-yl)methyl)-7-azaspiro[3.5]nonan-2-yl)carbamate (3)

A mixture of tert-butyl (4-bromo-2-fluorophenyl)carbamate (7 g, 24.13 mmol), benzyl (7-(piperidin-4-ylmethyl)-7-azaspiro[3.5]nonan-2-yl)carbamate (8.37 g, 20.51 mmol, HCl salt), Cs₂CO₃ (23.58 g, 72.38 mmol) and BrettPhos Pd G₃ (1.09 g, 1.21 mmol) in dioxane (100 mL) was degassed and purged with N₂ for 3 times, then the mixture was stirred at 100° C. for 16 hours under N₂ atmosphere. LCMS showed 38% of benzyl (7-(piperidin-4-ylmethyl)-7-azaspiro[3.5]nonan-2-yl)carbamate remained, a peak (30%) with desired mass. The reaction mixture was filtered and the filtrate was concentrated to give the residue. The residue was purified by flash silica gel chromatography (ISCO®; 20 g SepaFlash® Silica Flash Column, Eluent of 20-80% EtOAc/Petroleum ether gradient @ 80 mL/min) to afford benzyl (7-((1-(4-((tert-butoxycarbonyl)amino)-3-fluorophenyl)piperidin-4-yl)methyl)-7-azaspiro[3.5]nonan-2-yl)carbamate (2.5 g, 3.87 mmol, 16.06% yield, 90% purity) as a yellow solid. MS(M+H)⁺=581.5.

### Step 2. Synthesis of benzyl (7-((1-(4-amino-3-fluorophenyl)piperidin-4-yl)methyl)-7-azaspiro[3.5]nonan-2-yl) carbamate (4)

It was synthesized by similar method described in step 6 of Example 3 to obtain benzyl (7-((1-(4-amino-3-fluorophenyl)piperidin-4-yl)methyl)-7-azaspiro[3.5]nonan-2-yl)carb amate (2.2 g, 3.66 mmol, 85.06% yield, 80% purity) as a yellow solid. MS(M+H)⁺=481.3.

### Step 3. Synthesis of benzyl (7-((1-(4-((2,6-dioxopiperidin-3-yl)amino)-3-fluorophenyl)piperidin-4-yl)methyl)-7-azaspiro[3.5]nonan-2-yl)carbamate (6)

It was synthesized by similar method described in step 5 of Example 22 to obtain benzyl (7-((1-(4-((2,6-dioxopiperidin-3-yl)amino)-3-fluorophenyl)piperidin-4-yl)methyl)-7-azaspiro[3.5]nonan-2-yl)carbamate (2.3 g, 3.65 mmol, 99.78% yield, 94% purity) as a brown solid. MS(M+H)⁺=592.5.

### Step 4. Synthesis of 3-((4-(4-((2-amino-7-azaspiro[3.5]nonan-7-yl)methyl)piperidin-1-yl)-2-fluorophen yl)amino)piperidine-2,6-dione (7)

It was synthesized by similar method described in step 2 of Example 4 to obtain 3-((4-(4-((2-amino-7-azaspiro[3.5]nonan-7-yl)methyl)piperidin-1-yl)-2-fluorophenyl)amino)piperidine-2,6-dione (1.2 g, crude, TFA salt) as brown oil. MS(M+H)⁺=458.3.

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-3-fluoro phenyl)piperidin-4-yl)methyl)-7-azaspiro[3.5]nonan-2-yl)-3-methoxybenzamide (Compound 36)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-3-fluorophenyl)piperidin-4-yl)methyl)-7-azaspiro[3.5]nonan-2-yl)-3-methoxybenzamide (467.50 mg, 374.28 μmol, 41.50% yield, 98.5% purity, 3TFA) as gray solid. MS(M+H)⁺=887.6.

¹H NMR (400 MHz, DMSO-d₆) δ=10.83 (s, 1H), 9.15-8.98 (m, 1H), 8.54-8.48 (m, 1H), 8.29-8.13 (m, 3H), 7.53-7.47 (m, 2H), 7.24-6.79 (m, 3H), 4.83-4.73 (m, 1H), 4.47-4.36 (m, 2H), 4.12-4.03 (m, 2H), 3.94 (s, 3H), 3.59-3.51 (m, 2H), 3.47-3.36 (m, 2H), 3.33 (s, 3H), 3.10-3.04 (m, 2H), 3.00-2.91 (m, 2H), 2.89-2.70 (m, 2H), 2.62-2.54 (m, 1H), 2.41-2.34 (m, 1H), 2.21-2.13 (m, 1H), 2.09-1.67 (m, 16H), 1.65-1.43 (m, 6H).

### Example 37. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-3-fluoro phenyl)piperidin-4-yl)ethyl)piperidin-4-yl)-2-fluoro-5-methoxybenzamide (Compound 37)

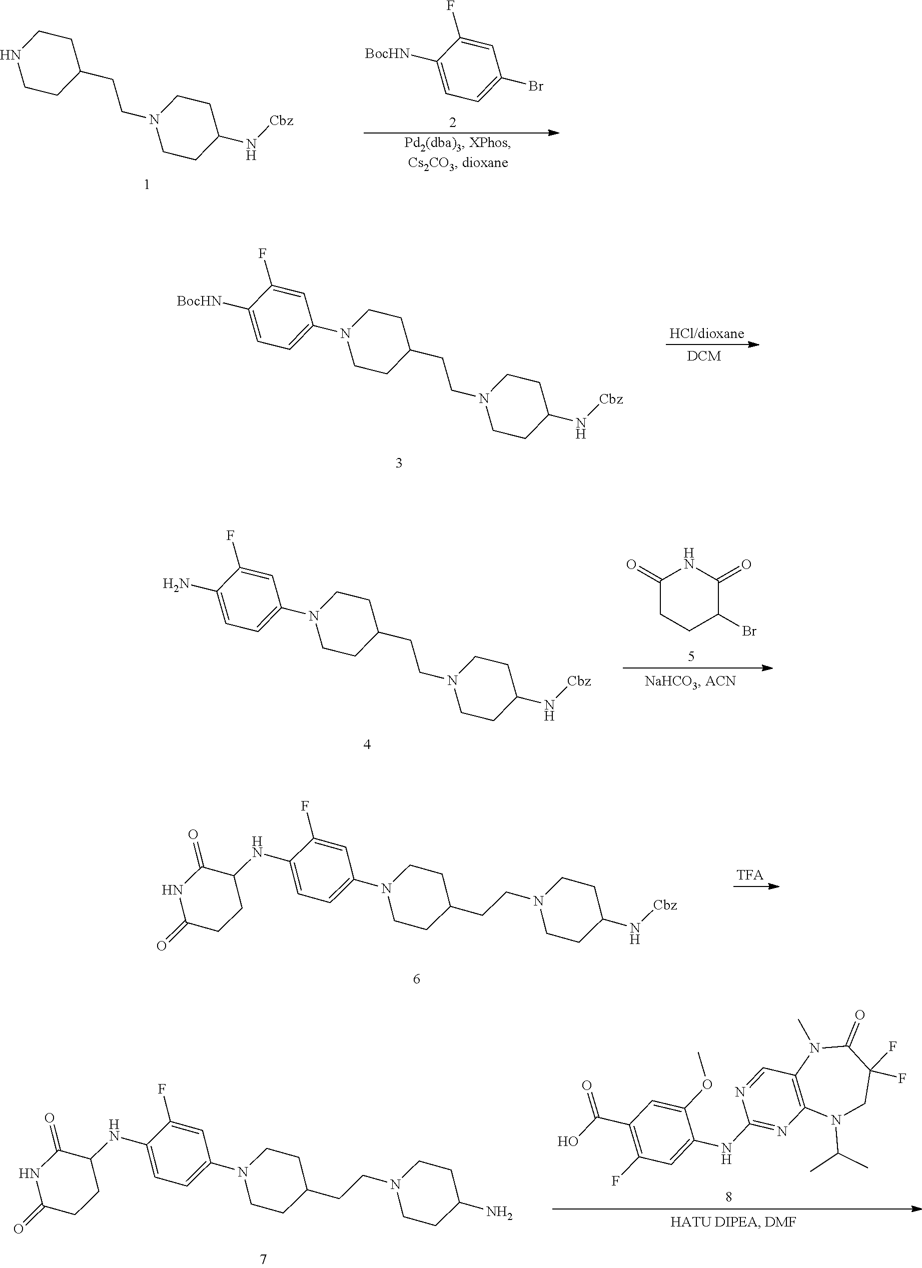

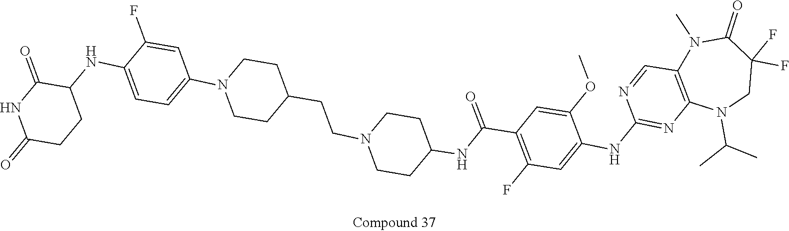 [SMILES: COc1cc(C(=O)NC2CCN(CCC3CCN(c4ccc(NC5CCC(=O)NC5=O)c(F)c4)CC3)CC2)c(F)cc1Nc1ncc2c(n1)N(C(C)C)CC(F)(F)C(=O)N2C]

### Step 1. Synthesis of tert-butyl (4-(4-(2-(4-(((benzyloxy)carbonyl)amino)piperidin-1-yl)ethyl)piperidin-1-yl)-2-fluorophenyl)carbamate (3)

It was synthesized by similar method described in step 3 of Example 3 to obtain tert-butyl (4-(4-(2-(4-(((benzyloxy)carbonyl)amino)piperidin-1-yl)ethyl)piperidin-1-yl)-2-fluoro phenyl)carbamate (3.3 g, 5.95 mmol, 64.92% yield) as a yellow solid. MS(M+H)⁺=555.4.

### Step 2. Synthesis of benzyl (1-(2-(1-(4-amino-3-fluorophenyl)piperidin-4-yl)ethyl)piperidin-4-yl)carbamate (4)

It was synthesized by similar method described in step 7 of Example 7 to obtain benzyl (1-(2-(1-(4-amino-3-fluorophenyl)piperidin-4-yl)ethyl)piperidin-4-yl)carbamate (3 g, crude, HCl) as a yellow solid. MS(M+H)⁺=455.2.

### Step 3. Synthesis of benzyl (1-(2-(1-(4-((2,6-dioxopiperidin-3-yl)amino)-3-fluorophenyl)piperidin-4-yl)ethyl)piperidin-4-yl)carbamate (6)

It was synthesized by similar method described in step 3 of Example 36 to obtain benzyl (1-(2-(1-(4-((2,6-dioxopiperidin-3-yl)amino)-3-fluorophenyl)piperidin-4-yl)ethyl)piperidin-4-yl)carbamate (1.5 g, 2.65 mmol, 52.08% yield, 100% purity) as a blue solid. MS(M+H)⁺=566.3.

### Step 4. Synthesis of 3-((4-(4-(2-(4-aminopiperidin-1-yl)ethyl)piperidin-1-yl)-2-fluorophenyl)amino)piperidine-2,6-dione (7)

It was synthesized by similar method described in step 4 of Example 36 to obtain 3-((4-(4-(2-(4-aminopiperidin-1-yl)ethyl)piperidin-1-yl)-2-fluorophenyl)amino)piperidine-2,6-dione (1.35 g, crude, TFA) as brown oil. MS(M+H)⁺=432.3.

### Step 5. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-3-fluoro phenyl)piperidin-4-yl)ethyl)piperidin-4-yl)-2-fluoro-5-methoxybenzamide (Compound 37)

It was synthesized by similar method described in step 5 of Example 36 to obtain 4-((7,7-difluoro-9-isopropyl-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-3-fluorophenyl)piperidin-4-yl)ethyl)piperidin-4-yl)-2-fluoro-5-methoxybenzamide (403.3 mg, 468.12 mol, 18.92% yield, 99% purity) as a white solid. MS(M+H)⁺=853.5.

¹H NMR (400 MHz, DMSO-d₆) δ=10.80 (s, 1H), 8.34-8.22 (m, 2H), 7.98 (s, 1H), 7.20 (d, J=6.6 Hz, 1H), 6.79-6.69 (m, 2H), 6.63-6.55 (m, 1H), 5.05 (d, J=6.5 Hz, 1H), 4.95-4.80 (m, 1H), 4.32-4.24 (m, 1H), 4.07 (t, J=13.4 Hz, 2H), 3.92 (s, 3H), 3.49-3.37 (m, 2H), 3.35-3.34 (m, 3H), 2.98-2.64 (m, 3H), 2.64-2.53 (m, 6H), 2.53-2.46 (m, 2H), 2.15-2.00 (m, 2H), 2.00-1.82 (m, 3H), 1.79-1.70 (m, 3H), 1.64-1.34 (m, 4H), 1.32-1.25 (m, 8H).

### Example 38. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)(methyl)amino)-2-fluorophenyl)piperazin-1-yl)ethyl)piperidin-1-yl)-3-methoxybenzamide (Compound 38)

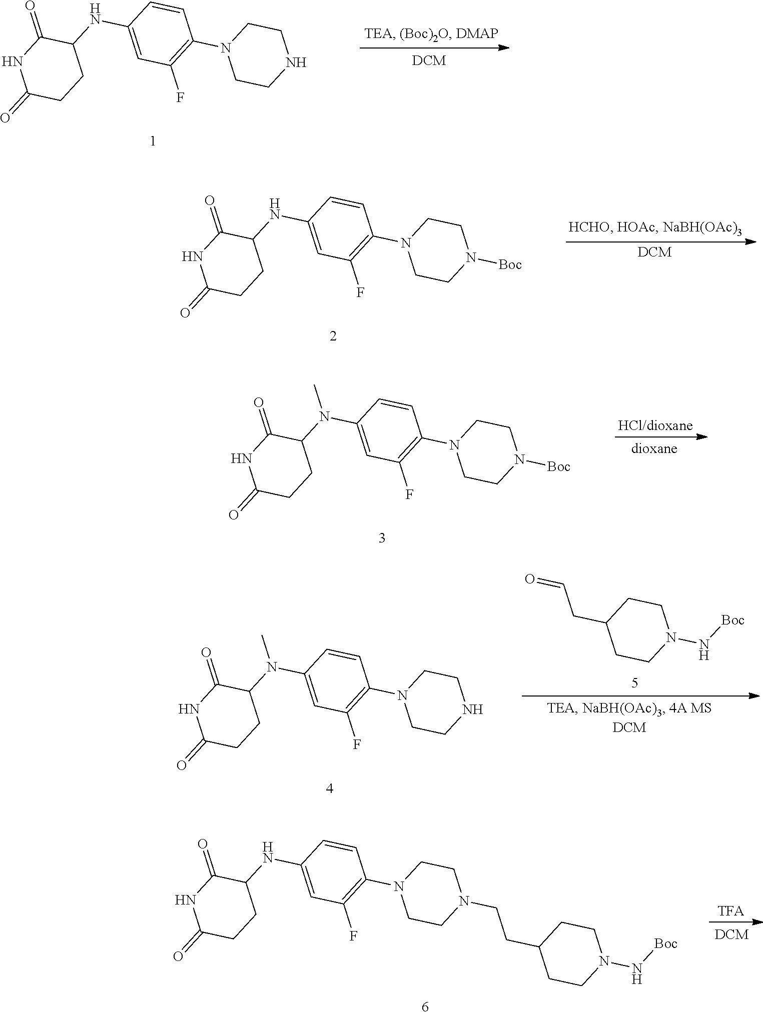 [SMILES: O=C1CCC(Nc2ccc(N3CCNCC3)c(F)c2)C(=O)N1 ; CC(C)(C)OC(=O)N1CCN(c2ccc(NC3CCC(=O)NC3=O)cc2F)CC1 ; CN(c1ccc(N2CCN(C(=O)OC(C)(C)C)CC2)c(F)c1)C1CCC(=O)NC1=O ; CN(c1ccc(N2CCNCC2)c(F)c1)C1CCC(=O)NC1=O ; CC(C)(C)OC(=O)NN1CCC(CC=O)CC1 ; CC(C)(C)OC(=O)NN1CCC(CCN2CCN(c3ccc(NC4CCC(=O)NC4=O)cc3F)CC2)CC1]

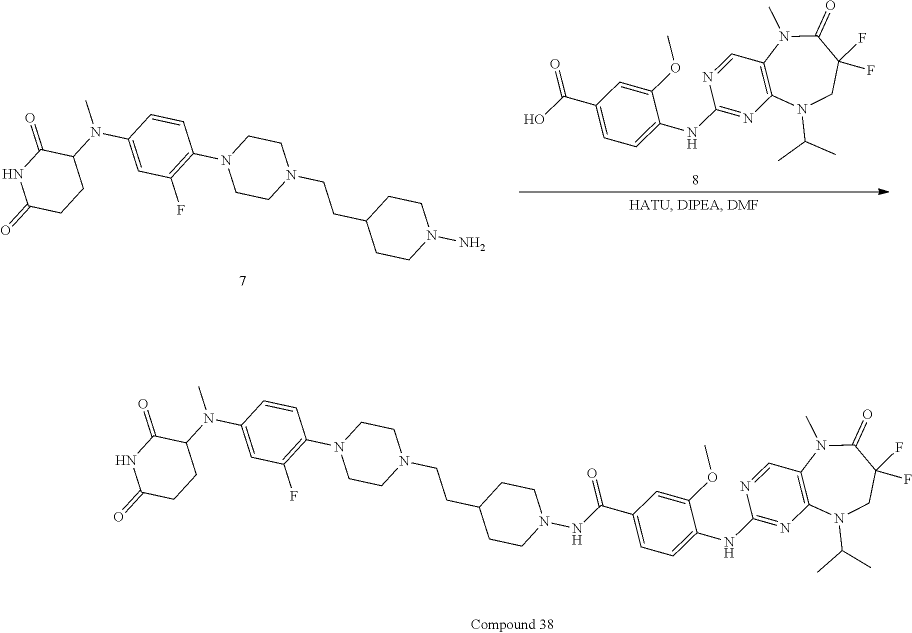 [SMILES: CN(c1ccc(N2CCN(CCC3CCN(N)CC3)CC2)c(F)c1)C1CCC(=O)NC1=O ; COc1cc(C(=O)O)ccc1Nc1ncc2c(n1)N(C(C)C)CC(F)(F)C(=O)N2C ; COc1cc(C(=O)NN2CCC(CCN3CCN(c4ccc(N(C)C5CCC(=O)NC5=O)cc4F)CC3)CC2)ccc1Nc1ncc2c(n1)N(C(C)C)CC(F)(F)C(=O)N2C]

### Step 1. Synthesis of tert-butyl 4-(4-((2,6-dioxopiperidin-3-yl)amino)-2-fluorophenyl)piperazine-1-carboxylate (2)

To a solution of 3-((3-fluoro-4-(piperazin-1-yl)phenyl)amino)piperidine-2,6-dione (2 g, 5.83 mmol, HCl salt) in DCM (30 mL) was added TEA (1.77 g, 17.50 mmol, 2.44 mL) and DMAP (71.28 mg, 583.44 μmol) and (Boc)₂O (1.53 g, 7.00 mmol, 1.61 mL). The mixture was stirred at 20° C. for 1.5 hr. LCMS showed 3-((3-fluoro-4-(piperazin-1-yl)phenyl)amino)piperidine-2,6-dione was consumed completely and one main peak with desired mass. The residue was diluted with water (30 mL) and extracted with DCM (30 mL×3). The combined organic layers were washed with water (30 mL), dried over Na₂SO₄, filtered and concentrated under reduced pressure to give a residue. The residue was purified by flash silica gel chromatography (25 g SepaFlash® Silica Flash Column, Eluent of 0-50% EtOAc/Petroleum ether gradient @ 100 mL/min) to afford tert-butyl 4-(4-((2,6-dioxopiperidin-3-yl)amino)-2-fluorophenyl)piperazine-1-carboxylate (1.8 g, 4.43 mmol, 75.91% yield) as a blue solid. MS(M+H)⁺=407.3.

### Step 2. Synthesis of tert-butyl 4-(4-((2,6-dioxopiperidin-3-yl)(methyl)amino)-2-fluorophenyl)piperazine-1-carboxylate (3)

To a solution of tert-butyl 4-(4-((2,6-dioxopiperidin-3-yl)amino)-2-fluorophenyl)piperazine-1-carboxylate (700 mg, 1.72 mmol) in DCM (10 mL) were added HCHO (698.90 mg, 8.61 mmol, 641.19 μL, 37% purity) and AcOH (103.42 mg, 1.72 mmol, 98.59 μL). After half an hour, NaBH(OAc)₃ (1.10 g, 5.17 mmol) was added to the reaction mixture. The mixture was stirred at 20° C. for 16 h. LCMS showed ~20% of tert-butyl 4-(4-((2,6-dioxopiperidin-3-yl)amino)-2-fluorophenyl)piperazine-1-carboxylate remained, several new peaks were shown on LCMS and ~73% of desired compound. HCHO (279.56 mg, 3.44 mmol, 256.48 μL, 37% purity) and NaBH(Oac)₃ (1.10 g, 5.17 mmol) were added to the reaction mixture. The mixture was stirred at 20° C. for 3 hr. LCMS showed tert-butyl 4-(4-((2,6-dioxopiperidin-3-yl)amino)-2-fluorophenyl)piperazine-1-carboxylate was consumed completely and one main peak with desired mas. The residue was diluted with NaHCO₃ solution (30 mL) and extracted with DCM (20 mL×3). The combined organic layers were washed with water (20 mL), dried over Na₂SO₄, filtered and concentrated under reduced pressure to give a residue. The residue was purified by flash silica gel chromatography (20 g SepaFlash® Silica Flash Column, Eluent of 0-50% EtOAc/Petroleum ether gradient @ 80 mL/min) to afford tert-butyl 4-(4-((2,6-dioxopiperidin-3-yl)(methyl)amino)-2-fluorophenyl)piperazine-1-carboxylate (720 mg, 1.71 mmol, 99.43% yield) was obtained as a blue solid. MS(M+H)⁺=421.2.

### Step 3. Synthesis of 3-((3-fluoro-4-(piperazin-1-yl)phenyl)(methyl)amino)piperidine-2,6-dione (4)

It was synthesized by similar method described in step 5 of Example 1 to obtain 3-((3-fluoro-4-(piperazin-1-yl)phenyl)(methyl)amino)piperidine-2,6-dione (305 mg, 854.77 mol, 99.84% yield, HCl salt) as a blue solid. MS(M+H)⁺=321.2.

### Step 4. Synthesis of tert-butyl (4-(2-(4-(4-((2,6-dioxopiperidin-3-yl)(methyl)amino)-2-fluorophenyl)piperazin-1-yl)ethyl)piperidin-1-yl)carbamate (6)

It was synthesized by similar method described in step 6 of Example 1 to obtain tert-butyl (4-(2-(4-(4-((2,6-dioxopiperidin-3-yl)(methyl)amino)-2-fluorophenyl)piperazin-1-yl)ethyl)piperidin-1-yl)carbamate (270 mg, 414.87 mol, 58.05% yield, 84% purity) as a light yellow oil. MS(M+H)⁺=547.5.

### Step 5. Synthesis of 3-((4-(4-(2-(1-aminopiperidin-4-yl)ethyl)piperazin-1-yl)-3-fluorophenyl)(methyl)amino)piperidine-2,6-dione (7)

It was synthesized by similar method described in step 6 of Example 3 to obtain 3-((4-(4-(2-(1-aminopiperidin-4-yl)ethyl)piperazin-1-yl)-3-fluorophenyl)(methyl)amin o)piperidine-2,6-dione (138 mg, 246.17 mol, 99.69% yield, TFA salt) as a yellow oil. MS(M+H)⁺=447.3.

### Step 6. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)(methyl)amino)-2-fluorophenyl)piperazin-1-yl)ethyl)piperidin-1-yl)-3-methoxybenzamide (Compound 38)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)(methyl)amino)-2-fluorophenyl)piperazin-1-yl)ethyl)piperidin-1-yl)-3-methoxybenzamide (32.85 mg, 37.88 μmol, 23.13% yield, 98% purity) as a white solid. MS(M+H)⁺=850.5.

¹H NMR (400 MHz, DMSO-d₆) δ=10.79 (s, 1H), 9.26 (s, 1H), 8.30 (d, J=8.1 Hz, 1H), 8.22 (s, 1H), 7.88 (s, 1H), 7.47-7.38 (m, 2H), 6.98-6.84 (m, 1H), 6.75-6.62 (m, 1H), 6.60-6.48 (m, 1H), 4.93-4.85 (m, 1H), 4.84-4.76 (m, 1H), 4.04 (t, J=13.6 Hz, 2H), 3.93 (s, 3H), 3.32-3.31 (m, 3H), 3.06-2.95 (m, 3H), 2.95-2.84 (m, 3H), 2.84-2.69 (m, 4H), 2.69 (s, 3H), 2.59-2.52 (m, 2H), 2.46-2.21 (m, 5H), 1.90-1.82 (m, 1H), 1.77-1.67 (m, 2H), 1.54-1.35 (m, 2H), 1.35-1.26 (m, 3H), 1.24 (d, J=6.7 Hz, 6H).

### Example 39. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-3-methoxybenzamide (Compound 39)

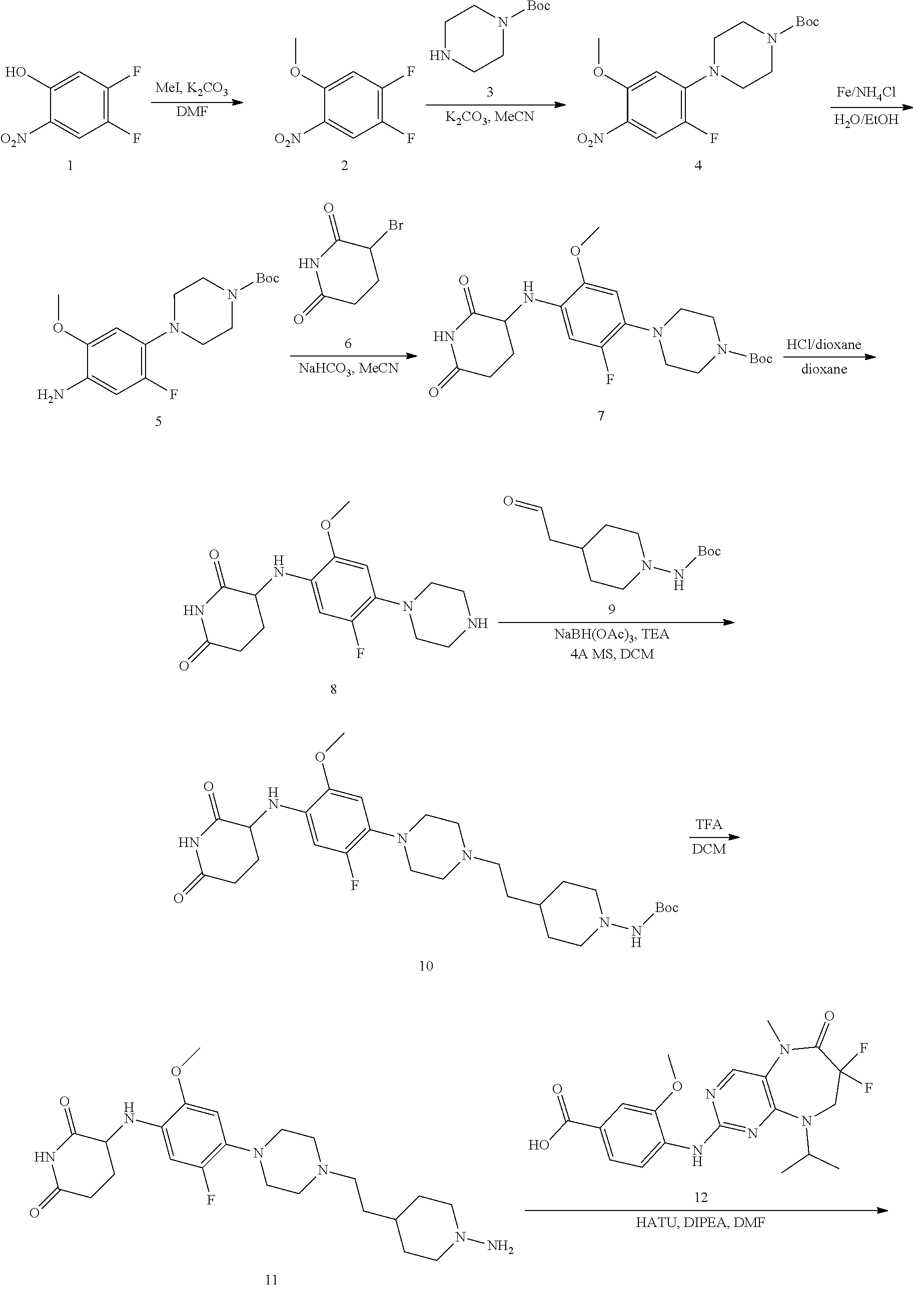

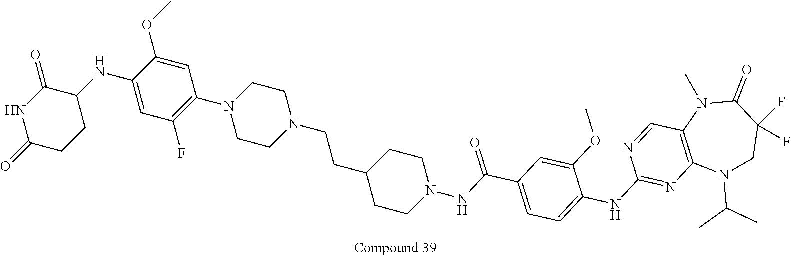 [SMILES: COc1cc(C(=O)NN2CCC(CCN3CCN(c4cc(OC)c(NC5CCC(=O)NC5=O)cc4F)CC3)CC2)ccc1Nc1ncc2c(n1)N(C(C)C)CC(F)(F)C(=O)N2C]

### Step 1. Synthesis of 1,2-difluoro-4-methoxy-5-nitrobenzene (2)

It was synthesized by similar method described in step 1 of Example 7 to obtain 1,2-difluoro-4-methoxy-5-nitrobenzene (2.3 g, 12.16 mmol, 85.18% yield) as a yellow solid. MS(M+H)⁺=190.1

### Step 2. Synthesis of tert-butyl 4-(2-fluoro-5-methoxy-4-nitrophenyl)piperazine-1-carboxylate (4)

It was synthesized by similar method described in step 4 of Example 30, changed DMF to MeCN (30 mL) to obtain tert-butyl 4-(2-fluoro-5-methoxy-4-nitrophenyl)piperazine-1-carboxylate (2.2 g, 6.19 mmol, 50.90% yield) as a yellow solid. MS(M+H)⁺=356.4

### Step 3. Synthesis of tert-butyl 4-(4-amino-2-fluoro-5-methoxyphenyl)piperazine-1-carboxylate (5)

It was synthesized by similar method described in step 4 of Example 22 to obtain tert-butyl 4-(4-amino-2-fluoro-5-methoxyphenyl)piperazine-1-carboxylate (1 g, 3.07 mmol, 99.29% yield) as a purple solid. MS(M+H)⁺=326.4

### Step 4. Synthesis of tert-butyl 4-(4-((2,6-dioxopiperidin-3-yl)amino)-2-fluoro-5-methoxyphenyl)piperazine-1-carboxylate (7)

It was synthesized by similar method described in step 5 of Example 22 to obtain tert-butyl 4-(4-((2,6-dioxopiperidin-3-yl)amino)-2-fluoro-5-methoxyphenyl)piperazine-1-carboxylate (1.02 g, 2.34 mmol, 95.05% yield) as a green solid. MS(M+H)⁺=437.2.

### Step 5. Synthesis of 3-((5-fluoro-2-methoxy-4-(piperazin-1-yl)phenyl)amino)piperidine-2,6-dione (8)

It was synthesized by similar method described in step 5 of Example 1 to obtain 3-((5-fluoro-2-methoxy-4-(piperazin-1-yl)phenyl)amino)piperidine-2,6-dione (550 mg, crude, HCl salt) as a blue solid. MS(M+H)⁺=337.1.

### Step 6. Synthesis of tert-butyl (4-(2-(4-(4-((2,6-dioxopiperidin-3-yl)amino)-2-fluoro-5-methoxyphenyl)piperazin-1-yl)ethyl)piperidin-1-yl)carbamate (10)

It was synthesized by similar method described in step 6 of Example 1 to obtain tert-butyl (4-(2-(4-(4-((2,6-dioxopiperidin-3-yl)amino)-2-fluoro-5-methoxyphenyl)piperazin-1-yl)ethyl)piperidin-1-yl)carbamate (340 mg, 604.26 mol, 45.06% yield) as a light yellow solid. MS(M+H)⁺=563.3.

### Step 7. Synthesis of 3-((4-(4-(2-(1-aminopiperidin-4-yl)ethyl)piperazin-1-yl)-5-fluoro-2-methoxyphenyl)amino)piperidine-2,6-dione (11)

It was synthesized by similar method described in step 6 of Example 3 to obtain 3-((4-(4-(2-(1-aminopiperidin-4-yl)ethyl)piperazin-1-yl)-5-fluoro-2-methoxyphenyl)amino)piperidine-2,6-dione (350 mg, crude, TFA salt) as a brown oil. MS(M+H)⁺=463.3.

### Step 8. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-3-methoxybenzamide (Compound 39)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-3-methoxybenzamide (120.4 mg, 133.48 μmol, 33.09% yield, 96% purity) as a white solid. MS(M+H)⁺=866.4.

¹H NMR (400 MHz, DMSO-d₆) δ=10.85 (s, 1H), 9.24 (s, 1H), 8.30 (d, J=7.8 Hz, 1H), 8.22 (s, 1H), 7.87 (s, 1H), 7.49-7.37 (m, 2H), 6.61 (d, J=7.9 Hz, 1H), 6.54 (d, J=14.5 Hz, 1H), 5.08 (d, J=6.4 Hz, 1H), 4.93-4.82 (m, 1H), 4.31-4.20 (m, 1H), 4.03 (t, J=13.5 Hz, 2H), 3.93 (s, 3H), 3.79 (s, 3H), 3.30 (s, 3H), 3.08-2.94 (m, 3H), 2.94-2.86 (m, 4H), 2.85-2.69 (m, 4H), 2.42-2.26 (m, 4H), 2.16-2.09 (m, 1H), 1.97-1.88 (m, 1H), 1.76-1.68 (m, 2H), 1.46-1.38 (m, 2H), 1.37-1.27 (m, 4H), 1.24 (d, J=6.7 Hz, 6H).

### Example 40. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-3-methoxybenzamide (Compound 40)

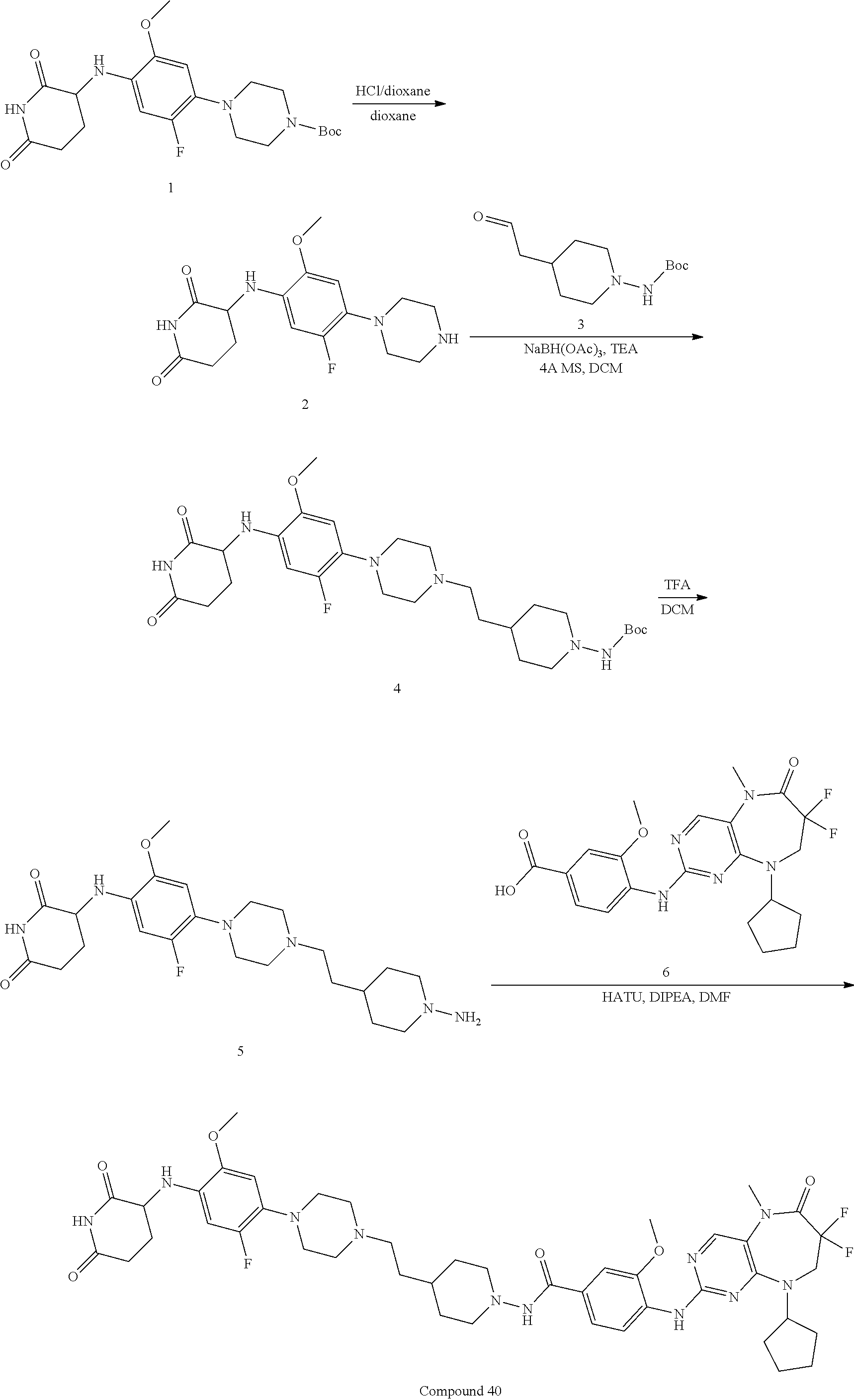

### Step 1. Synthesis of 3-((5-fluoro-2-methoxy-4-(piperazin-1-yl)phenyl)amino)piperidine-2,6-dione (2)

To a solution of tert-butyl 4-(4-((2,6-dioxopiperidin-3-yl)amino)-2-fluoro-5-methoxyphenyl)piperazine-1-carboxylate (500 mg, 1.15 mmol) in dioxane (1 mL) was added HCl/dioxane (4 M, 5 mL). The mixture was stirred at 20° C. for 3 hr. LCMS showed tert-butyl 4-(4-((2,6-dioxopiperidin-3-yl)amino)-2-fluoro-5-methoxyphenyl)piperazine-1-carboxylate was consumed completely. The reaction mixture was concentrated under reduced pressure to remove solvent to afford 3-((5-fluoro-2-methoxy-4-(piperazin-1-yl)phenyl)amino)piperidine-2,6-dione (500 mg, crude, HCl salt) as a blue solid. MS(M+H)⁺=337.1.

### Step 2. Synthesis of tert-butyl (4-(2-(4-(4-((2,6-dioxopiperidin-3-yl)amino)-2-fluoro-5-methoxyphenyl)piperazin-1-yl)ethyl)piperidin-1-yl)carbamate (4)

To a solution of 3-((5-fluoro-2-methoxy-4-(piperazin-1-yl)phenyl)amino)piperidine-2,6-dione (500 mg, 1.34 mmol, HCl salt) and tert-butyl (4-(2-oxoethyl)piperidin-1-yl)carbamate (487.46 mg, 2.01 mmol) in DCM (10 mL) was added TEA (135.71 mg, 1.34 mmol, 186.67 μL) and 4A MS (50 mg, 134.11 μmol). After half an hour, NaBH(OAc)₃ (852.72 mg, 4.02 mmol) was added to the reaction mixture. The mixture was stirred at 20° C. for 16 hr. LCMS showed 3-((5-fluoro-2-methoxy-4-(piperazin-1-yl)phenyl)amino)piperidine-2,6-dione was consumed completely and one main peak with desired mass. The residue was diluted with NaHCO₃ solution (30 mL) and extracted with DCM (20 mL×3). The combined organic layers were washed with water (20 mL), dried over Na₂SO₄, filtered and concentrated under reduced pressure to give a residue. The residue was purified by flash silica gel chromatography (20 g SepaFlash® Silica Flash Column, Eluent of 10-20% MeOH/EtOAc ether gradient @ 80 mL/min) to afford tert-butyl (4-(2-(4-(4-((2,6-dioxopiperidin-3-yl)amino)-2-fluoro-5-methoxyphenyl)piperazin-1-yl)ethyl)piperidin-1-yl)carbamate (390 mg, 693.12 μmol, 51.68% yield) as a light yellow solid. MS(M+H)⁺=563.3.

### Step 3. Synthesis of 3-((4-(4-(2-(1-aminopiperidin-4-yl)ethyl)piperazin-1-yl)-5-fluoro-2-methoxyphenyl)amino)piperidine-2,6-dione (5)

To a solution of tert-butyl (4-(2-(4-(4-((2,6-dioxopiperidin-3-yl)amino)-2-fluoro-5-methoxyphenyl)piperazin-1-yl)ethyl)piperidin-1-yl)carbamate (390 mg, 693.12 mol) in DCM (6 mL) was added TFA (3.07 g, 26.93 mmol, 2 mL). The mixture was stirred at 20° C. for 1 hr. LCMS showed ~3% of tert-butyl (4-(2-(4-(4-((2,6-dioxopiperidin-3-yl)amino)-2-fluoro-5-methoxyphenyl)piperazin-1-yl)ethyl)piperidin-1-yl)carbamate remained and ~75% of desired mass. The reaction mixture was concentrated under reduced pressure to remove solvent to afford 3-((4-(4-(2-(1-aminopiperidin-4-yl)ethyl)piperazin-1-yl)-5-fluoro-2-methoxyphenyl)amino)piperidine-2,6-dione (410 mg, crude, TFA salt) as a brown oil. MS(M+H)⁺=463.2.

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-3-methoxybenzamide (Compound 40)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (210 mg, 469.34 μmol) in DMF (4 mL) were added HATU (267.69 mg, 704.01 mol), DIPEA (303.29 mg, 2.35 mmol, 408.75 μL) and 3-((4-(4-(2-(1-aminopiperidin-4-yl)ethyl)piperazin-1-yl)-5-fluoro-2-methoxyphenyl)amino)piperidine-2,6-dione (405.92 mg, 704.01 mol, TFA salt). The mixture was stirred at 20° C. for 2 hr. LCMS showed 3-((4-(4-(2-(1-aminopiperidin-4-yl)ethyl)piperazin-1-yl)-5-fluoro-2-methoxyphenyl)amino)piperidine-2,6-dione was consumed completely and one main peak with desired mass. The mixture was diluted with water (30 mL) and extracted with EtOAc (30 mL×3). The combined organic layers were washed with brine (30 mL×3), dried over Na₂SO₄, filtered and concentrated under reduced pressure to give a residue. The residue was purified by flash silica gel chromatography (ISCO®; 20 g SepaFlash® Silica Flash Column, Eluent of 10-20% MeOH/EtOAc gradient @ 80 mL/min) and repurified by prep-HPLC (column: Waters Xbridge C18 150×50 mm×10 μm; mobile phase: [water (NH₄HCO₃)-ACN]; gradient: 34%-64% B over 10 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-m ethoxyphenyl)piperazin-1-yl)ethyl)piperidin-1-yl)-3-methoxybenzamide (111.7 mg, 123.47 mol, 26.31% yield, 98.6% purity) as a pink solid. MS(M+H)⁺=892.5.

¹H NMR (400 MHz, DMSO-d₆) δ=10.96-10.73 (m, 1H), 9.28 (s, 1H), 8.30-8.21 (m, 2H), 7.96 (s, 1H), 7.45-7.37 (m, 2H), 6.60 (d, J=8.2 Hz, 1H), 6.54 (d, J=14.4 Hz, 1H), 5.09 (d, J=6.5 Hz, 1H), 4.83-4.69 (m, 1H), 4.30-4.19 (m, 1H), 4.05 (t, J=14.0 Hz, 2H), 3.93 (s, 3H), 3.79 (s, 3H), 3.33-3.32 (m, 3H), 3.04-2.96 (m, 2H), 2.95-2.86 (m, 4H), 2.85-2.72 (m, 3H), 2.62-2.51 (m, 4H), 2.40-2.30 (m, 3H), 2.16-2.09 (m, 1H), 1.99-1.88 (m, 3H), 1.75-1.66 (m, 4H), 1.64-1.55 (m, 4H), 1.47-1.38 (m, 2H), 1.36-1.24 (m, 3H).

### Example 41. Synthesis of N(4-(2-(4-(4-((2,6-dioxopiperidin-3-yl)amino)-2-fluoro-5-methoxyphenyl)piperazin-1-yl)ethyl)piperidin-1-yl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (Compound 41)

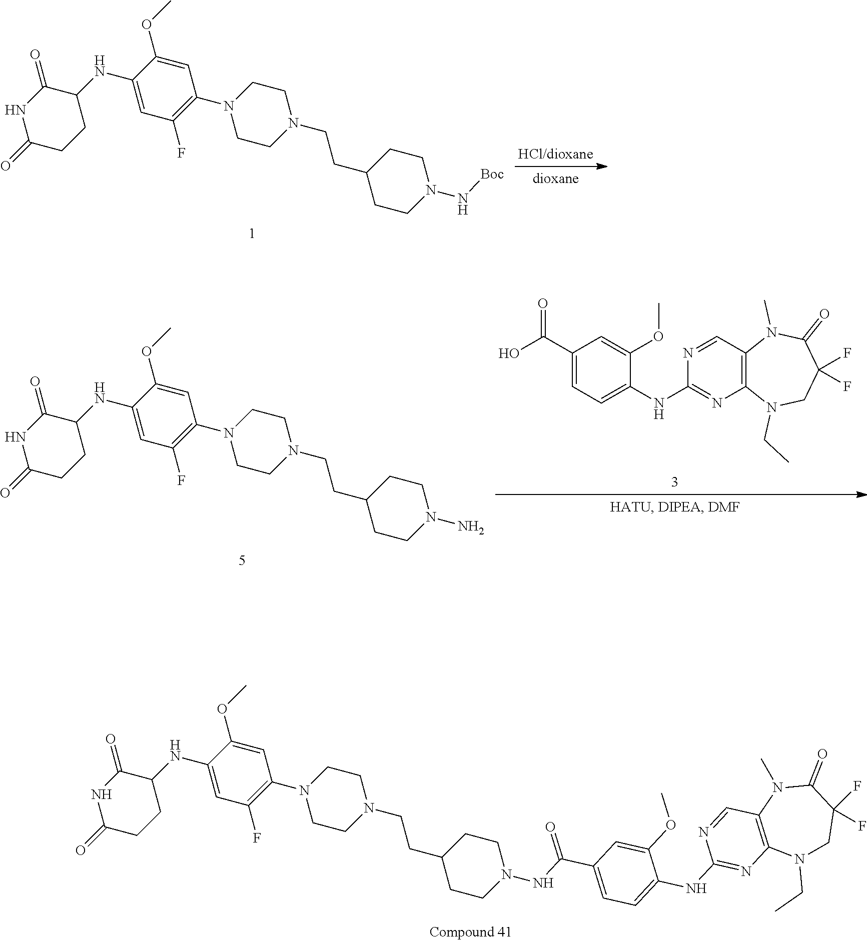 [SMILES: COc1cc(N2CCN(CCC3CCN(NC(=O)OC(C)(C)C)CC3)CC2)c(F)cc1NC1CCC(=O)NC1=O ; COc1cc(N2CCN(CCC3CCN(N)CC3)CC2)c(F)cc1NC1CCC(=O)NC1=O ; CCN1CC(F)(F)C(=O)N(C)c2cnc(Nc3ccc(C(=O)O)cc3OC)nc21 ; CCN1CC(F)(F)C(=O)N(C)c2cnc(Nc3ccc(C(=O)NN4CCC(CCN5CCN(c6cc(OC)c(NC7CCC(=O)NC7=O)cc6F)CC5)CC4)cc3OC)nc21]

### Step 1. Synthesis of 3-((4-(4-(2-(1-aminopiperidin-4-yl)ethyl)piperazin-1-yl)-5-fluoro-2-methoxyphenyl)amino)piperidine-2,6-dione (2)

It was synthesized by similar method described in step 5 of Example 1 to obtain 3-((4-(4-(2-(1-aminopiperidin-4-yl)ethyl)piperazin-1-yl)-5-fluoro-2-methoxyphenyl)amino)piperidine-2,6-dione (1.15 g, HCl salt) as a light brown solid which was used directly. MS(M+H)⁺=463.2.

### Step 2. Synthesis of N(4-(2-(4-(4-((2,6-dioxopiperidin-3-yl)amino)-2-fluoro-5-methoxyphenyl)piperazin-1-yl)ethyl)piperidin-1-yl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (Compound 41)

It was synthesized by similar method described in step 10 of Example 2 to obtain N(4-(2-(4-(4-((2,6-dioxopiperidin-3-yl)amino)-2-fluoro-5-methoxyphenyl)piperazin-1-yl)ethyl)piperidin-1-yl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (78.7 mg, 64.53 mol, 13.14% yield, 97.9% purity, 3TFA salt) as an off-white solid. MS(M+H)⁺=852.3.

¹H NMR (400 MHz, DMSO-d₆) δ=10.49 (s, 1H), 9.92-9.69 (m, 1H), 9.66-9.36 (m, 1H), 8.42-8.30 (m, 1H), 8.28-8.12 (m, 2H), 7.51-7.33 (m, 2H), 6.69-6.45 (m, 2H), 4.31-4.19 (m, 3H), 3.94 (s, 3H), 3.81 (s, 3H), 3.68-3.64 (m, 2H), 3.57 (d, J=11.4 Hz, 2H), 3.35-3.30 (m, 5H), 3.25-3.11 (m, 6H), 3.08-2.98 (m, 2H), 2.87-2.84 (m, 2H), 2.82-2.73 (m, 1H), 2.59-2.56 (m, 1H), 2.15-2.06 (m, 1H), 2.01-1.89 (m, 1H), 1.85-1.75 (m, 2H), 1.70-1.60 (m, 2H), 1.45-1.30 (m, 3H), 1.21 (t, J=7.0 Hz, 3H).

### Example 42. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-2-fluoro-5-methoxybenz amide (Compound 42)

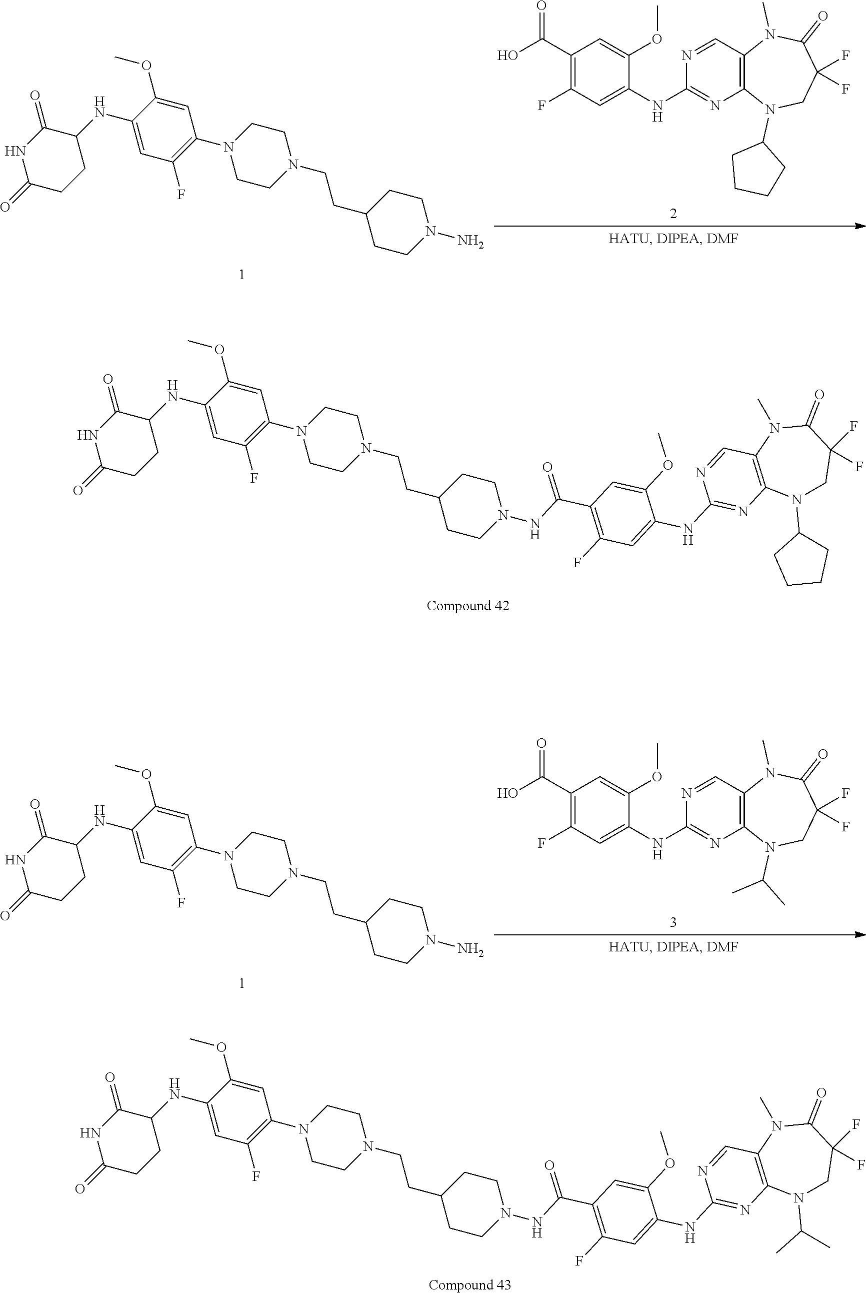 [SMILES: COc1cc(N2CCN(CCC3CCN(N)CC3)CC2)c(F)cc1NC1CCC(=O)NC1=O ; COc1cc(C(=O)O)c(F)cc1Nc1ncc2c(n1)N(C1CCCC1)CC(F)(F)C(=O)N2C ; COc1cc(C(=O)NN2CCC(CCN3CCN(c4cc(OC)c(NC5CCC(=O)NC5=O)cc4F)CC3)CC2)c(F)cc1Nc1ncc2c(n1)N(C1CCCC1)CC(F)(F)C(=O)N2C ; COc1cc(N2CCN(CCC3CCN(N)CC3)CC2)c(F)cc1NC1CCC(=O)NC1=O ; COc1cc(C(=O)O)c(F)cc1Nc1ncc2c(n1)N(C(C)C)CC(F)(F)C(=O)N2C ; COc1cc(C(=O)NN2CCC(CCN3CCN(c4cc(OC)c(NC5CCC(=O)NC5=O)cc4F)CC3)CC2)c(F)cc1Nc1ncc2c(n1)N(C(C)C)CC(F)(F)C(=O)N2C]

To the solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (1.61 g, 3.47 mmol) in DMF (30 mL) were added HATU (1.58 g, 4.16 mmol) and DIPEA (1.34 g, 10.41 mmol, 1.81 mL) and the resulting mixture was stirred at 20° C. for 1 h, then 3-((4-(4-(2-(1-aminopiperidin-4-yl)ethyl)piperazin-1-yl)-5-fluoro-2-methoxyphenyl)amino)piperidine-2,6-dione (2 g, 3.47 mmol, TFA salt) was added the resulting mixture was stirred at 20° C. for 2 h. LCMS showed a peak (66%) with desired mass. The reaction mixture was diluted with water (100 mL) and extracted with EtOAc (50 mL×3). The combined organic layers were washed with saturated brine (100 mL), dried over anhydrous Na₂SO₄, filtered and concentrated under reduced pressure. The residue was purified by flash silica gel chromatography (ISCO®; 40 g SepaFlash® Silica Flash Column, Eluent of 0-10% MeOH/EtOAc gradient @ 100 mL/min) to afford 1.7 g impure product, which was triturated with ACN (50 mL) and filtered. The filter cake was collected to afford 1.6 g of desired product as a brown solid. The product was re-purified by prep-HPLC (column: Phenomenex luna C18 150*40 mm*15 um; mobile phase: [water (TFA)−ACN]; gradient: 25%-55% B over 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-m ethoxyphenyl)piperazin-1-yl)ethyl)piperidin-1-yl)-2-fluoro-5-methoxybenzamide (1.16 g, 1.12 mmol, 32.20% yield, 99% purity, TFA salt) as a white solid. MS(M+H)⁺=910.4.

¹H NMR (400 MHz, DMSO-d₆) δ=10.86 (s, 1H), 9.61-9.49 (m, 1H), 9.25 (br s, 1H), 8.31 (s, 1H), 8.24 (d, J=13.0 Hz, 1H), 8.15 (s, 1H), 7.16 (d, J=6.5 Hz, 1H), 6.66-6.56 (m, 2H), 4.87-4.80 (m, 1H), 4.29 (br dd, J=4.8, 11.9 Hz, 1H), 4.10 (br t, J=13.9 Hz, 2H), 3.92 (s, 3H), 3.82 (s, 3H), 3.57 (br d, J=11.1 Hz, 2H), 3.38-3.31 (m, 5H), 3.23-3.03 (m, 8H), 2.87-2.68 (m, 3H), 2.61-2.54 (m, 1H), 2.16-2.07 (m, 1H), 2.02-1.91 (m, 3H), 1.82-1.60 (m, 10H), 1.44-1.25 (m, 3H).

### Example 43. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-2-fluoro-5-methoxybenzamide (Compound 43)

It was synthesized by similar method described in step 1 of Example 42 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-2-fluoro-5-methoxybenzamide (803 mg, 796.60 μmol, 20.88% yield, 99% purity, TFA salt) as a yellow solid. MS(M+H)⁺=884.4.

¹H NMR (400 MHz, DMSO-d₆) δ=10.86 (s, 1H), 9.66-9.48 (m, 1H), 9.23 (br s, 1H), 8.34-8.20 (m, 2H), 8.11 (s, 1H), 7.16 (d, J=6.6 Hz, 1H), 6.70-6.50 (m, 2H), 4.93-4.88 (m, 1H), 4.29 (br dd, J=5.1, 11.9 Hz, 1H), 4.09 (br t, J=13.3 Hz, 2H), 3.92 (s, 3H), 3.82 (s, 3H), 3.57 (br d, J=11.6 Hz, 2H), 3.34 (s, 5H), 3.26-2.99 (m, 8H), 2.85-2.66 (m, 3H), 2.57 (br s, 1H), 2.16-2.06 (m, 1H), 2.02-1.87 (m, 1H), 1.82-1.57 (m, 4H), 1.45-1.30 (m, 3H), 1.27 (d, J=6.6 Hz, 6H).

### Example 44. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-2-fluoro-3-methoxybenz amide (Compound 44)

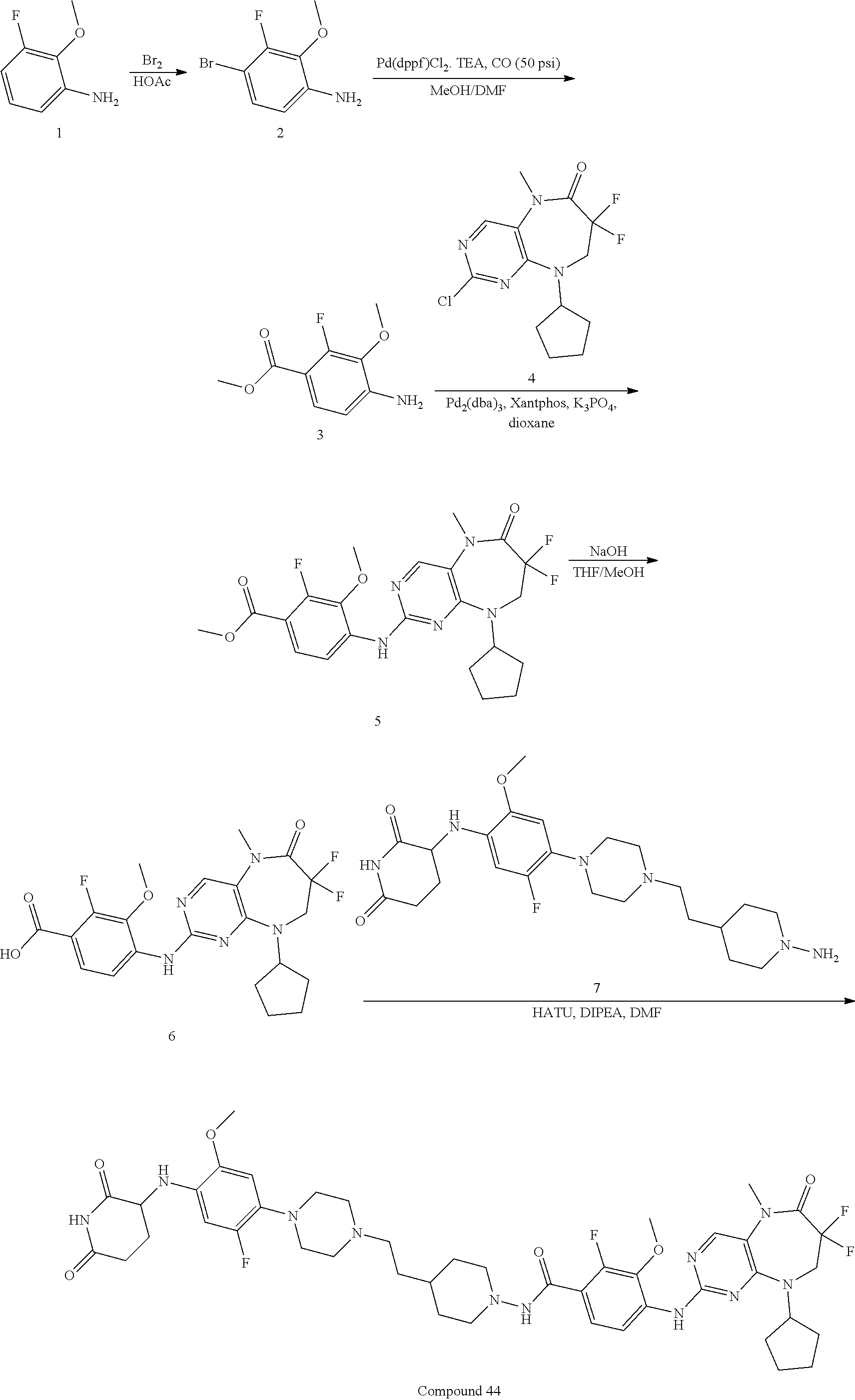

### Step 1. Synthesis of 4-bromo-3-fluoro-2-methoxyaniline (2)

To a solution of 3-fluoro-2-methoxyaniline (8 g, 56.68 mmol) in AcOH (30 mL) was added a mixture of Br₂ (7.25 g, 45.34 mmol, 2.34 mL) in AcOH (30 mL) dropwise, the resulting mixture was stirred at 20° C. for 0.5 hrs. TLC (SiO₂, Petroleum ether:EtOAc=5:1) indicated trace of the starting material remained and a main spot with larger polarity. The suspension was filtered and the filter cake was washed with Petroleum ether (50 mL) and a mixture (Petroleum ether:EtOAc=3:1, 20 mL), the filter cake was collected and dried in vacuum and was purified by flash silica gel chromatography (20 g SepaFlash® Silica Flash Column, Eluent of 3-10% EtOAc/Petroleum ether gradient @ 200 mL/min), the eluent was concentrated in vacuum to afford 4-bromo-3-fluoro-2-methoxyaniline (8 g, 29.09 mmol, 51.32% yield, 80% purity) as an off-white solid. MS(M+H)⁺=220.0.

### Step 2. Synthesis of methyl 4-amino-2-fluoro-3-methoxybenzoate (3)

To a solution of 4-bromo-3-fluoro-2-methoxyaniline (8 g, 29.09 mmol) and TEA (23.99 g, 237.09 mmol, 33 mL) in MeOH (100 mL) and DMF (50 mL) was added Pd(dppf)Cl₂·CH₂Cl₂ (2.7 g, 3.31 mmol) under N₂ atmosphere. The suspension was degassed under vacuum and purged with CO several times. The mixture was stirred under CO (50 psi) atmosphere at 80° C. for 120 hrs. LCMS showed the starting material was consumed completely and 40% of desired mass. The mixture was filtered and the filter cake was washed with MeOH (300 mL), the filtrate was concentrated in vacuum to remove the MeOH, the residue was diluted with brine (200 mL) and extracted with EtOAc (100 mL×3). The combined organic layers were washed with brine (200 mL×4), dried over Na₂SO₄, filtered and concentrated in vacuum. The residue was purified by flash silica gel chromatography (80 g SepaFlash® Silica Flash Column, Eluent of 10~12% EtOAc/Petroleum ether gradient @ 200 mL/min) to afford methyl 4-amino-2-fluoro-3-methoxybenzoate (3.13 g, 15.71 mmol, 54.03% yield) as an off-white solid. MS(M+H)⁺=200.4.

¹H NMR (400 MHz, DMSO-d₆) δ=7.36 (t, J=8.3 Hz, 1H), 6.49 (dd, J=1.1, 8.8 Hz, 1H), 6.09 (s, 2H), 3.74 (d, J=5.4 Hz, 6H).

### Step 3. Synthesis of methyl 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-3-methoxybenzoate (5)

A mixture of 2-chloro-9-cyclopentyl-7,7-difluoro-5-methyl-5,7,8,9-tetrahydro-6H-pyrimido[4,5-b][1,4]diazepin-6-one (6 g, 18.94 mmol), methyl 4-amino-2-fluoro-3-methoxybenzoate (3.30 g, 16.57 mmol), Pd₂(dba)₃ (1 g, 1.09 mmol), Xantphos (1 g, 1.73 mmol) and K₃ PO₄ (12.00 g, 56.53 mmol) in dioxane (100 mL) was stirred at 100° C. for 16 hours under N₂ atmosphere. LCMS showed 2-chloro-9-cyclopentyl-7,7-difluoro-5-methyl-5,7,8,9-tetrahydro-6H-pyrimido[4,5-b][1,4]diazepin-6-one was consumed completely and 64% of desired mass. The mixture was diluted with brine (300 mL) and extracted with EtOAc (150 mL×3). The combined organic layers were dried over Na₂SO₄, filtered and concentrated in vacuum. The residue was purified by flash silica gel chromatography (20 g SepaFlash® Silica Flash Column, Eluent of 15-35% EtOAc/Petroleum ether gradient @ 200 mL/min) to afford methyl 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-3-methoxybenzoate (6.17 g, 12.87 mmol, 67.93% yield) as a light brown solid. MS(M+H)⁺=480.3.

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-3-methoxybenzoic acid (6)

A mixture of methyl 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-3-methoxybenzoate (6.17 g, 12.87 mmol) and NaOH (2 M, 30 mL) in MeOH (30 mL) and THF (30 mL) was stirred at 20° C. for 12 hrs. LCMS showed the starting material was consumed completely and desired mass. The reaction mixture was concentrated in vacuum to remove the organic solvent, to the residue was added HCl solution (12 M) to adjust pH<2. To the suspension was added ACN (20 mL) and EtOAc (20 mL), the resulting mixture was stirred at 15° C. for 10 minutes, the suspension was filtered and the filter cake was washed with EtOAc (20 mL), the filter cake was collected and dried. The filter cake was diluted with HCl solution (12 M, 30 mL) and concentrated in vacuum at 75° C. The residue was triturated with a mixture (MeCN:EtOAc=1:3, 60 mL) for 10 minutes, the suspension was filtered and the filter cake was washed with a mixture (MeCN:EtOAc=1:3, 40 mL), the filter cake was collected and dried in vacuum to afford 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-3-methoxybenzoic acid (5.64 g, 12.12 mmol, 94.16% yield) as an off-white solid. MS(M+H)⁺=466.3.

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-2-fluoro-3-methoxybenz amide (Compound 44)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-m ethoxyphenyl)piperazin-1-yl)ethyl)piperidin-1-yl)-2-fluoro-3-methoxybenzamide (522.2 mg, 567.84 μmol, 61.82% yield, 98.95% purity) as a white solid. MS(M+H)⁺=910.5.

¹H NMR (400 MHz, DMSO-d₆) δ=10.58 (s, 1H), 8.92-8.62 (m, 1H), 8.27 (s, 1H), 8.16-7.95 (m, 2H), 7.34-6.98 (m, 1H), 6.64 (d, J=8.1 Hz, 1H), 6.55 (d, J=14.1 Hz, 1H), 5.01 (d, J=6.4 Hz, 1H), 4.87-4.67 (m, 1H), 4.29-4.17 (m, 1H), 4.02 (t, J=14.0 Hz, 2H), 3.95 (s, 3H), 3.82 (s, 3H), 3.36 (s, 3H), 3.04-2.88 (m, 6H), 2.86-2.77 (m, 1H), 2.76-2.63 (m, 2H), 2.63-2.57 (m, 2H), 2.56 (s, 3H), 2.45-1.99 (m, 2H), 2.25-2.16 (m, 1H), 1.98 (dd, J=4.7, 10.9 Hz, 2H), 1.95-1.87 (m, 1H), 1.82-1.68 (m, 4H), 1.67-1.57 (m, 4H), 1.53-1.41 (m, 2H), 1.41-1.27 (m, 3H).

### Example 45. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-5-fluoro-2-methoxybenzamide (Compound 45)

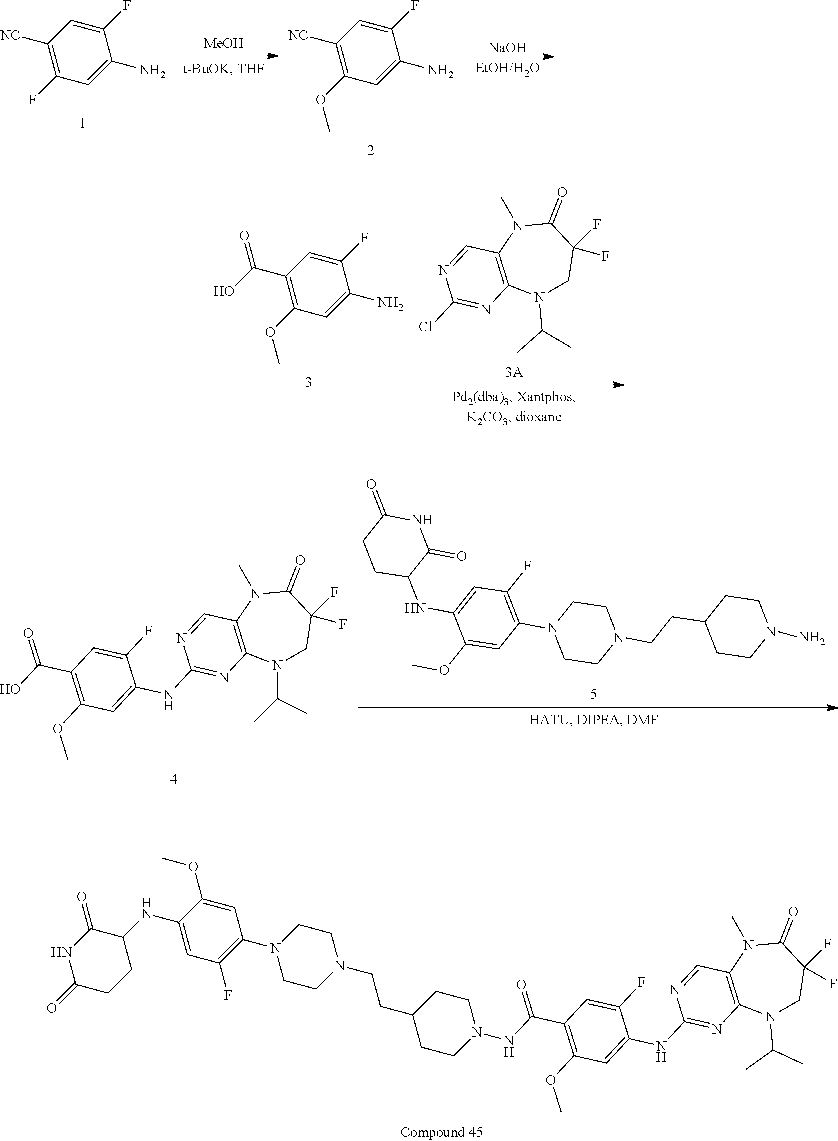

### Step 1. Synthesis of 4-amino-5-fluoro-2-methoxybenzonitrile (2)

To a solution of MeOH (23.75 g, 741.35 mmol, 30.00 mL) in THF (100 mL) was added t-BuOK (1 M, 160.00 mL) dropwise at 0° C. under N₂ atmosphere, the mixture was stirred at 20° C. for 0.5 hours. Then 4-amino-2,5-difluorobenzonitrile (10 g, 64.89 mmol) was added, the resulting mixture was stirred at 70° C. for 3 hours. LCMS showed the starting material was consumed completely and 99% of desired mass. The mixture was concentrated in vacuum to remove most of the solvent. The residue was diluted with H₂O (300 mL), extracted with MTBE (150 mL×4). The combined organic layers were dried over Na₂SO₄, filtered and concentrated in vacuum. The residue was purified by flash silica gel chromatography (40 g SepaFlash® Silica Flash Column, Eluent of 8-28% EtOAc/Petroleum ether gradient @ 200 mL/min) to afford 4-amino-5-fluoro-2-methoxybenzonitrile (9.85 g, 59.28 mmol, 91.37% yield) as a gray solid. MS(M+H)⁺=167.5.

### Step 2. Synthesis of 4-amino-5-fluoro-2-methoxybenzoic acid (3)

A mixture of 4-amino-5-fluoro-2-methoxybenzonitrile (2.98 g, 17.94 mmol), NaOH (8 M, 16 mL) and EtOH (15 mL) in a smothered can was stirred at 110° C. for 20 hrs. LCMS showed the starting material was consumed completely and desired mass was detected. After cooling to room temperature, 3 batches of the mixture were combined and concentrated in vacuum to remove most of the solvent, the residue was partitioned between water (100 mL) and EtOAc (100 mL). The aqueous layer was acidified by HCl solution (6 M) until pH=4, the resulting mixture was extracted with a mixture (EtOAc:MeOH=20:1, 200 mL×5), dried over Na₂SO₄, filtered and concentrated in vacuum. The residue was purified by column (DCM:MeOH=10:1) to afford 4-amino-5-fluoro-2-methoxybenzoic acid (10.73 g) as a light brown solid, which was used directly. MS(M+H)⁺=186.1.

### Step 3. Synthesis of 4-((7,7-difluoro-9-isopropyl-5-methyl-6-oxo-6,7,8,9-tetrahydro-5H-pyrimido[4,5-b][1,4]diazepin-2-yl)amino)-5-fluoro-2-methoxybenzoic acid (4)

It was synthesized by similar method described in step 3 of Example 44, changed K₃ PO₄ to K₂CO₃ (4.00 g, 28.94 mmol) to obtain 4-((7,7-difluoro-9-isopropyl-5-methyl-6-oxo-6,7,8,9-tetrahydro-5H-pyrimido[4,5-b][1,4]diazepin-2-yl)amino)-5-fluoro-2-methoxybenzoic acid (3.15 g, 7.17 mmol, 52.10% yield) as a gray solid. MS(M+H)⁺=440.3.

¹H NMR (400 MHz, DMSO-d₆) δ=9.64 (s, 1H), 8.30 (s, 1H), 7.71 (d, J=6.6 Hz, 1H), 7.59 (d, J=11.1 Hz, 1H), 5.04-4.93 (m, 1H), 4.17 (t, J=12.8 Hz, 2H), 3.85 (s, 3H), 3.37 (s, 3H), 1.21 (d, J=6.7 Hz, 6H).

### Step 4. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-5-fluoro-2-methoxybenzamide (Compound 45)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-5-fluoro-2-methoxybenzamide (660.01 mg, 727.64 μmol, 53.42% yield, 97.45% purity) as a light pink solid. MS(M+H)⁺=884.5.

¹H NMR (400 MHz, DMSO-d₆) δ=10.69-10.44 (m, 1H), 8.79-8.67 (m, 1H), 8.64-8.40 (m, 1H), 8.20 (s, 1H), 7.79 (d, J=6.4 Hz, 1H), 7.56-7.41 (m, 1H), 6.63 (d, J=8.1 Hz, 1H), 6.54 (d, J=14.3 Hz, 1H), 5.00 (d, J=6.4 Hz, 1H), 4.97-4.80 (m, 1H), 4.29-4.16 (m, 1H), 3.98 (t, J=13.4 Hz, 2H), 3.90 (s, 3H), 3.80 (s, 3H), 3.34 (s, 3H), 3.01 (d, J=1.1 Hz, 2H), 2.99-2.88 (m, 4H), 2.86-2.76 (m, 1H), 2.76-2.63 (m, 2H), 2.62-2.53 (m, 2H), 2.54-2.50 (m, 3H), 2.43-2.35 (m, 2H), 2.24-2.15 (m, 1H), 1.96-1.85 (m, 1H), 1.79-1.64 (m, 2H), 1.50-1.40 (m, 2H), 1.40-1.26 (m, 3H), 1.20 (d, J=6.6 Hz, 6H).

### Example 46. Synthesis of 5-((7,7-difluoro-9-isopropyl-5-methyl-6-oxo-8H-pyrimido[4,5-b][1,4]diazepin-2-yl) amino)-N-(4-(2-(4-(4-((2,6-dioxo-3-piperidyl)amino)-2-fluoro-5-methoxy-phenyl)piperazin-1-yl)ethyl)-1-piperidyl)-4-methoxy-pyridine-2-carboxamide (Compound 46)

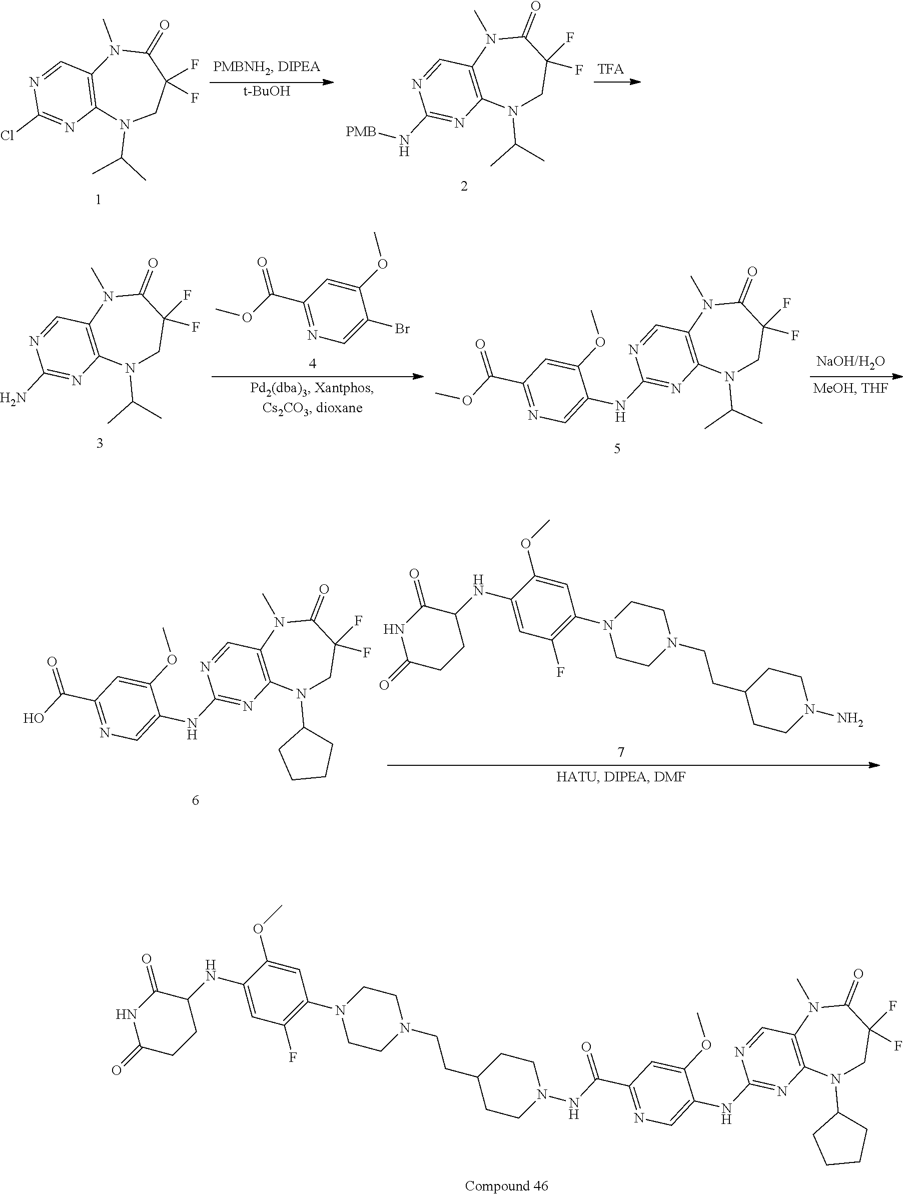

### Step 1. Synthesis of 7,7-difluoro-9-isopropyl-2-((4-methoxybenzyl)amino)-5-methyl-5,7,8,9-tetrahydro-6H-pyrimido[4,5-b][1,4]diazepin-6-one (2)

To a solution of 2-chloro-7,7-difluoro-9-isopropyl-5-methyl-8H-pyrimido[4,5-b][1,4]diazepin-6-one (5.00 g, 17.20 mmol) and (4-methoxyphenyl)methanamine (3.54 g, 25.80 mmol) in t-BuOH (5 mL) was added DIPEA (4.45 g, 34.40 mmol). The mixture was stirred at 100° C. for 26 hours. LCMS showed ~74% of desired compound. The reaction mixture was concentrated under reduced pressure to remove solvent. The residue was diluted with water (60 mL) then extracted with EtOAc (50 mL×3). The combined organic layers were washed with brine (20 mL×3), dried over Na₂SO₄, filtered and concentrated under reduced pressure to give a residue. The residue was triturated with MTBE (30 mL) at 25° C. for 10 min to afford 7,7-difluoro-9-isopropyl-2-((4-methoxybenzyl)amino)-5-methyl-5,7,8,9-tetrahydro-6H-pyrimido[4,5-b][1,4]diazepin-6-one (5.20 g, 7.97 mmol, 46.34% yield, 60% purity). MS(M+H)⁺=392.2.

### Step 2. Synthesis of 2-amino-7,7-difluoro-9-isopropyl-5-methyl-8H-pyrimido[4,5-b][1,4]diazepin-6-one (3)

A mixture of 7,7-difluoro-9-isopropyl-2-((4-methoxybenzyl)amino)-5-methyl-5,7,8,9-tetrahydro-6H-pyrimido[4,5-b][1,4]diazepin-6-one (5.20 g, 7.97 mmol) in TFA (50 mL) was stirred at 25° C. for 16 hours. LCMS showed ~51% of desired compound. The reaction mixture was concentrated under reduced pressure to remove TFA. Then the residue was diluted with EtOAc (200 mL) and washed with saturated NaHCO₃ solution (30 mL×2), dried over Na₂SO₄, filtered and concentrated under reduced pressure to give a residue. The residue was triturated with MTBE (30 mL) at 25° C. for 10 min to afford 2-amino-7,7-difluoro-9-isopropyl-5-methyl-8H-pyrimido[4,5-b][1,4]diazepin-6-one (2.30 g, 5.94 mmol, 74.46% yield, 70% purity) as a pale yellow solid. MS(M+H)⁺=272.1.

### Step 3. Synthesis of methyl 5-((7,7-difluoro-9-isopropyl-5-methyl-6-oxo-8H-pyrimido[4,5-b][1,4]diazepin-2-yl) amino)-4-methoxy-pyridine-2-carboxylate (5)

It was synthesized by similar method described in step 3 of Example 3 to obtain methyl 5-((7,7-difluoro-9-isopropyl-5-methyl-6-oxo-8H-pyrimido[4,5-b][1,4]diazepin-2-yl)amino)-4-methoxy-pyridine-2-carboxylate (1.25 g, 2.72 mmol, 62.03% yield, 95% purity) as a pale yellow solid. MS(M+H)⁺=437.2.

### Step 4. Synthesis of 5-((7,7-difluoro-9-isopropyl-5-methyl-6-oxo-8H-pyrimido[4,5-b][1,4]diazepin-2-yl) amino)-4-methoxy-pyridine-2-carboxylic acid (6)

To a solution of methyl 5-((7,7-difluoro-9-isopropyl-5-methyl-6-oxo-8H-pyrimido[4,5-b][1,4]diazepin-2-yl)amino)-4-methoxy-pyridine-2-carboxylate (1.25 g, 2.72 mmol) in MeOH (10 mL) and THF (10 mL) was added NaOH/H₂O (2 M, 10 mL). The mixture was stirred at 25° C. for 16 hours. LCMS showed ~91% of desired compound. The mixture was adjust pH=2 with HCl/dioxane (2 M), then concentrated to afford 5-((7,7-difluoro-9-isopropyl-5-methyl-6-oxo-8H-pyrimido[4,5-b][1,4]diazepin-2-yl)amino)-4-methoxy-pyridine-2-carboxylic acid (2 g, crude) as a yellow solid. The crude product was used directly without further purification. MS(M+H)⁺=423.2.

### Step 5. Synthesis of 5-((7,7-difluoro-9-isopropyl-5-methyl-6-oxo-8H-pyrimido[4,5-b][1,4]diazepin-2-yl) amino)-N-(4-(2-(4-(4-((2,6-dioxo-3-piperidyl)amino)-2-fluoro-5-methoxy-phenyl)piperazin-1-yl)ethyl)-1-piperidyl)-4-methoxy-pyridine-2-carboxamide (Compound 46)

It was synthesized by similar method described in step 10 of Example 2 to obtain 5-((7,7-difluoro-9-isopropyl-5-methyl-6-oxo-8H-pyrimido[4,5-b][1,4]diazepin-2-yl)amino)-N-(4-(2-(4-(4-((2,6-dioxo-3-piperidyl)amino)-2-fluoro-5-methoxy-phenyl)piperazin-I-yl)ethyl)-1-piperidyl)-4-methoxy-pyridine-2-carboxamide (702 mg, 795.18 μmol, 27.99% yield, 98.2% purity) as a pale yellow solid. MS(M+H)⁺=867.4.

¹H NMR (400 MHz, DMSO-d₆) δ=10.85 (s, 1H), 9.35 (s, 1H), 9.10 (s, 1H), 8.21-8.16 (m, 2H), 7.62 (s, 1H), 6.10 (d, J=8.0 Hz, 1H), 6.54 (d, J=14.4 Hz, w1H), 5.13-5.05 (m, 1H), 4.85-4.74 (m, 1H), 4.30-4.20 (m, 1H), 4.08-3.95 (m, 5H), 3.79 (s, 3H), 3.32-3.29 (m, 5H), 3.00-2.85 (m, 6H), 2.85-2.75 (m, 1H), 2.75-2.65 (m, 2H), 2.59-2.54 (m, 1H), 2.40-2.30 (m, 2H), 2.17-2.08 (m, 1H), 1.98-1.84 (m, 1H), 1.77-1.65 (m, 2H), 1.47-1.37 (m, 2H), 1.35-1.31 (m, 3H), 1.29-1.23 (m, 1H), 1.19 (d, J=6.4 Hz, 6H).

### Example 47. Synthesis of 5-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-4-methoxypicolinamide (Compound 47)

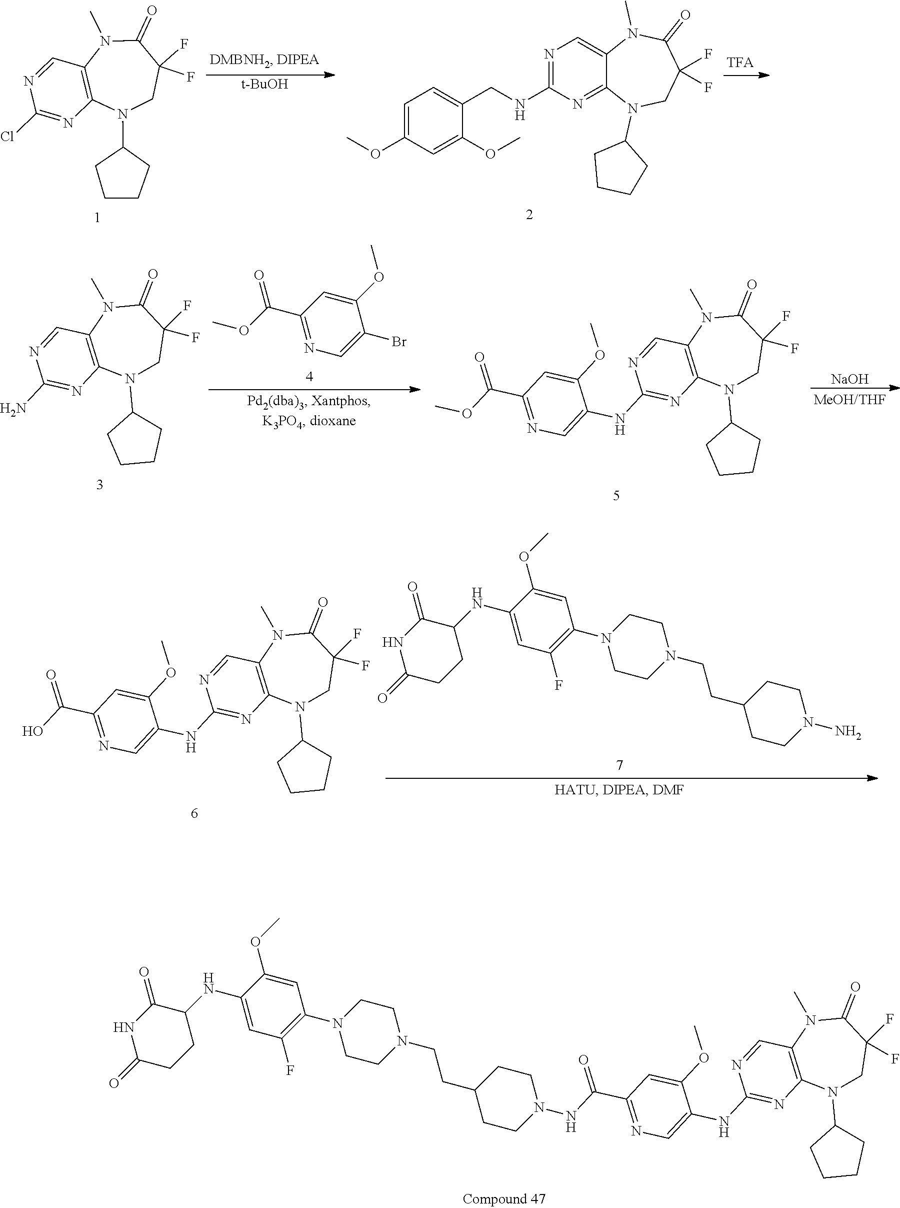

### Step 1. Synthesis of 9-cyclopentyl-2-((2,4-dimethoxybenzyl)amino)-7,7-difluoro-5-methyl-5,7,8,9-tetra hydro-6H-pyrimido[4,5-b][1,4]diazepin-6-one (2)

It was synthesized by similar method described in step 1 of Example 46, changed PMBNH₂ to DMBNH₂ (13.36 g, 79.88 mmol, 12.00 mL) to obtain 9-cyclopentyl-2-((2,4-dimethoxybenzyl)amino)-7,7-difluoro-5-methyl-5,7,8,9-tetrahydro-6H-pyrimido[4,5-b][1,4]diazepin-6-one (13 g, crude) as a yellow solid. MS(M+H)⁺=448.3.

### Step 2. Synthesis of 2-amino-9-cyclopentyl-7,7-difluoro-5-methyl-5,7,8,9-tetrahydro-6H-pyrimido[4,5-b][1,4]diazepin-6-one (3)

It was synthesized by similar method described in step 2 of Example 46 to obtain 2-amino-9-cyclopentyl-7,7-difluoro-5-methyl-5,7,8,9-tetrahydro-6H-pyrimido[4,5-b][1,4]diazepin-6-one (6.13 g, 20.21 mmol, 69.55% yield, 98% purity) as a yellow solid. MS(M+H)⁺=298.2.

### Step 3. Synthesis of methyl 5-((9-cyclopentyl-7,7-difluoro-5-methyl-6-oxo-6,7,8,9-tetrahydro-5H-pyrimido[4,5-b][1,4]diazepin-2-yl)amino)-4-methoxypicolinate (5)

It was synthesized by similar method described in step 3 of Example 46, changed Cs₂ CO₃ to K₃PO₄ (12.96 g, 61.08 mmol) to obtain methyl 5-((9-cyclopentyl-7,7-difluoro-5-methyl-6-oxo-6,7,8,9-tetrahydro-5H-pyrimido[4,5-b][1,4]diazepin-2-yl)amino)-4-methoxypicolinate (7.61 g, 16.13 mmol, 79.51% yield, 98% purity) as a yellow solid. MS(M+H)⁺=463.2.

### Step 4. Synthesis of 5-((9-cyclopentyl-7,7-difluoro-5-methyl-6-oxo-6,7,8,9-tetrahydro-5H-pyrimido[4,5-b][1,4]diazepin-2-yl)amino)-4-methoxypicolinic acid (6)

It was synthesized by similar method described in step 4 of Example 46 to obtain 5-((9-cyclopentyl-7,7-difluoro-5-methyl-6-oxo-6,7,8,9-tetrahydro-5H-pyrimido[4,5-b][1,4]diazepin-2-yl)amino)-4-methoxypicolinic acid (6.46 g, 13.11 mmol, 79.66% yield, 91% purity) as a white solid. MS(M+H)⁺=449.3.

### Step 5. Synthesis of 5-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methoxyphenyl)piperazin-1-yl)ethyl)piperidin-1-yl)-4-methoxypicolinamide (Compound 47)

It was synthesized by similar method described in step 5 of Example 46 to obtain 5-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-m ethoxyphenyl)piperazin-1-yl)ethyl)piperidin-1-yl)-4-methoxypicolinamide (149.8 mg, 166.41 μmol, 4.97% yield, 99% purity) as a yellow solid. MS(M+H)⁺=893.4.

¹H NMR (400 MHz, DMSO-d₆) δ=10.84 (s, 1H), 9.33 (s, 1H), 9.01 (s, 1H), 8.36 (s, 1H), 8.22 (s, 1H), 7.62 (s, 1H), 6.61 (d, J=8.1 Hz, 1H), 6.54 (d, J=14.5 Hz, 1H), 5.08 (d, J=6.4 Hz, 1H), 4.70-4.58 (m, 1H), 4.30-4.20 (m, 1H), 4.06-3.93 (m, 5H), 3.79 (s, 3H), 3.32 (s, 3H), 3.31-3.25 (m, 4H), 3.01-2.87 (m, 6H), 2.84-2.75 (m, 1H), 2.72-264 (m, 2H), 2.58-2.53 (m, 1H), 2.35 (t, J=7.1 Hz, 2H), 2.17-2.08 (m, 1H), 1.96-1.80 (m, 3H), 1.75-1.61 (m, 4H), 1.59-1.47 (m, 4H), 1.45-1.38 (m, 2H), 1.36-1.26 (m, 3H).

### Example 48. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2-fluoro-5-methoxyphenyl)piperazin-1-yl)ethyl)piperidin-4-yl)-3-methoxybenzamide (Compound 48)

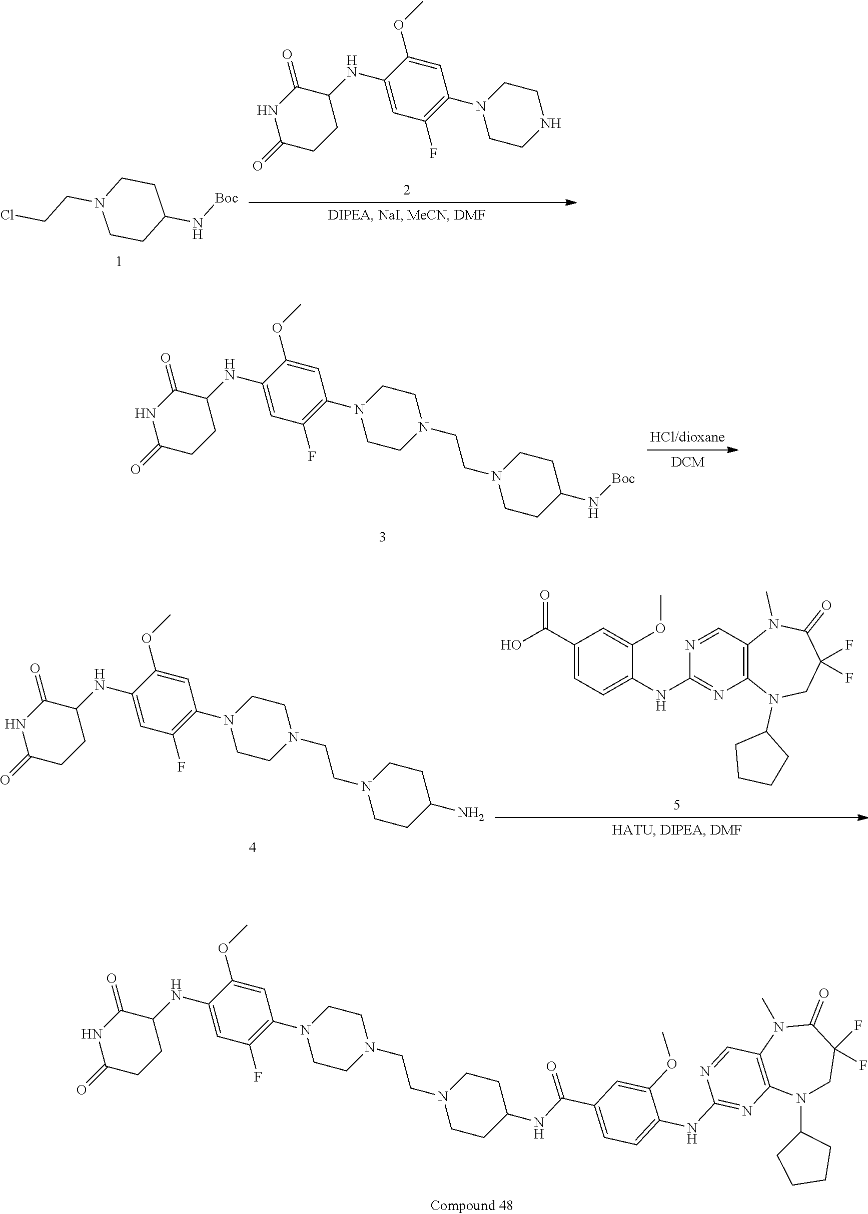 [SMILES: COc1cc(N2CCNCC2)c(F)cc1NC1CCC(=O)NC1=O ; CC(C)(C)OC(=O)NC1CCN(CCCl)CC1 ; COc1cc(N2CCN(CCN3CCC(NC(=O)OC(C)(C)C)CC3)CC2)c(F)cc1NC1CCC(=O)NC1=O ; COc1cc(N2CCN(CCN3CCC(N)CC3)CC2)c(F)cc1NC1CCC(=O)NC1=O ; COc1cc(C(=O)O)ccc1Nc1ncc2c(n1)N(C1CCCC1)CC(F)(F)C(=O)N2C ; COc1cc(C(=O)NC2CCN(CCN3CCN(c4cc(OC)c(NC5CCC(=O)NC5=O)cc4F)CC3)CC2)ccc1Nc1ncc2c(n1)N(C1CCCC1)CC(F)(F)C(=O)N2C]

### Step 1. Synthesis of tert-butyl (1-(2-(4-(4-((2,6-dioxopiperidin-3-yl)amino)-2-fluoro-5-methoxyphenyl)piperazin-1-yl)ethyl)piperidin-4-yl)carbamate (3)

It was synthesized by similar method described in step 3 of Example 23, addition MeCN (24 mL) to obtain tert-butyl (1-(2-(4-(4-((2,6-dioxopiperidin-3-yl)amino)-2-fluoro-5-methoxyphenyl)piperazin-1-yl)ethyl)piperidin-4-yl)carbamate (1.59 g, 2.77 mmol, 34.41% yield, 98% purity) as a gray solid. MS(M+H)⁺=563.4.

### Step 2. Synthesis of 3-((4-(4-(2-(4-aminopiperidin-1-yl)ethyl)piperazin-1-yl)-5-fluoro-2-methoxyphenyl)amino)piperidine-2,6-dione (4)

It was synthesized by similar method described in step 5 of Example 1, changed dioxane to DCM (10 mL) to obtain 3-((4-(4-(2-(4-aminopiperidin-1-yl)ethyl)piperazin-1-yl)-5-fluoro-2-methoxyphenyl)amino)piperidine-2,6-dione (1.4 g, crude, HCl) as a gray solid. MS(M+H)⁺=463.3.

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2-fluoro-5-methoxyphenyl)piperazin-1-yl)ethyl)piperidin-4-yl)-3-methoxybenzamide (Compound 48)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2-fluoro-5-m ethoxyphenyl)piperazin-1-yl)ethyl)piperidin-4-yl)-3-methoxybenzamide (1.65 g, 1.84 mmol, 68.49% yield, 99.4% purity) as a yellow solid. MS(M+H)⁺=892.6.

¹H NMR (400 MHz, DMSO-d₆) δ=10.84 (s, 1H), 8.31-8.22 (m, 2H), 8.18-8.06 (m, 1H), 7.96 (s, 1H), 7.53-7.43 (m, 2H), 6.64-6.49 (m, 2H), 5.09 (d, J=6.7 Hz, 1H), 4.82-4.69 (m, 1H), 4.29-4.21 (m, 1H), 4.04 (t, J=13.9 Hz, 2H), 3.94 (s, 3H), 3.84-3.72 (m, 4H), 3.34-3.33 (m, 6H), 2.96-2.86 (m, 6H), 2.83-2.73 (m, 1H), 2.64-2.52 (m, 6H), 2.16-1.86 (m, 6H), 1.84-1.51 (m, 10H).

### Example 49. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2-fluoro-5-methoxyphenyl)piperazin-1-yl)ethyl)piperidin-4-yl)-2-fluoro-5-methoxybenz amide (Compound 49)

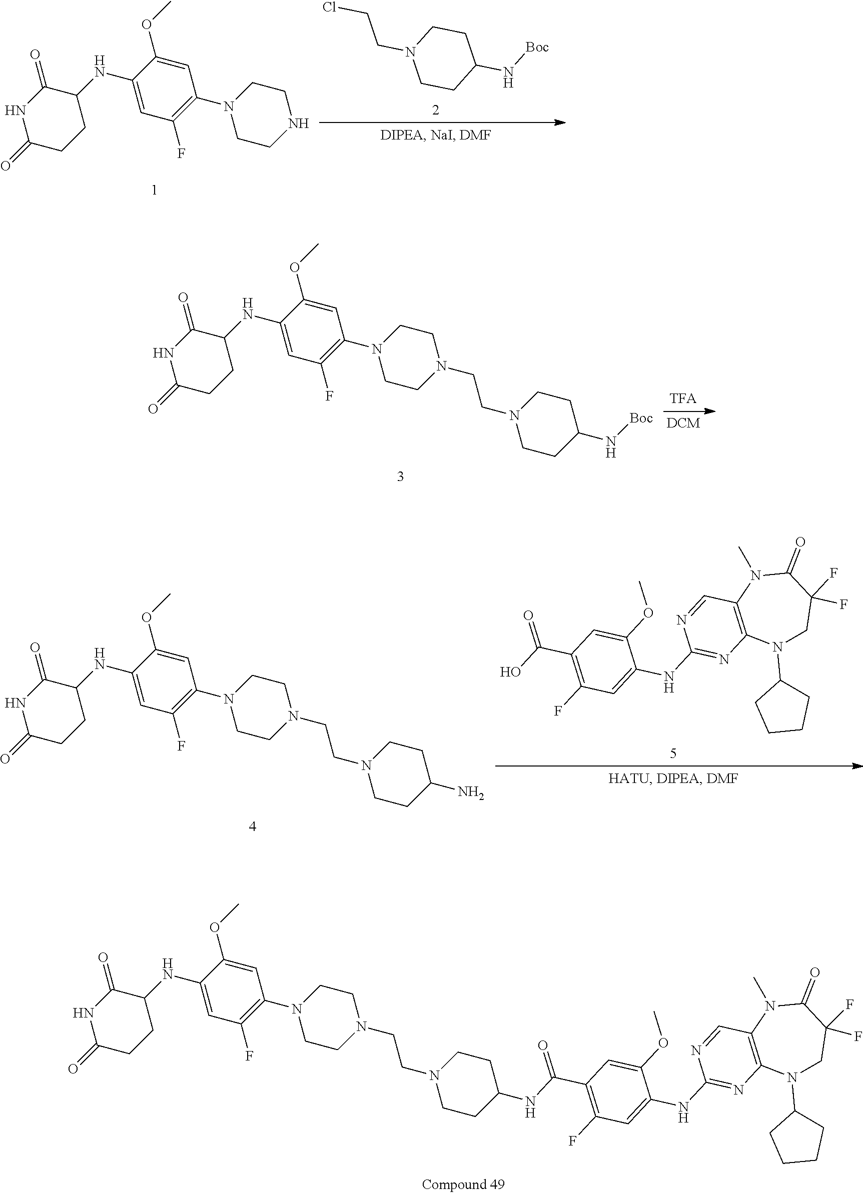 [SMILES: COc1cc(N2CCNCC2)c(F)cc1NC1CCC(=O)NC1=O ; CC(C)(C)OC(=O)NC1CCN(CCCl)CC1 ; COc1cc(N2CCN(CCN3CCC(NC(=O)OC(C)(C)C)CC3)CC2)c(F)cc1NC1CCC(=O)NC1=O ; COc1cc(N2CCN(CCN3CCC(N)CC3)CC2)c(F)cc1NC1CCC(=O)NC1=O ; COc1cc(C(=O)O)c(F)cc1Nc1ncc2c(n1)N(C1CCCC1)CC(F)(F)C(=O)N2C ; COc1cc(C(=O)NC2CCN(CCN3CCN(c4cc(OC)c(NC5CCC(=O)NC5=O)cc4F)CC3)CC2)c(F)cc1Nc1ncc2c(n1)N(C1CCCC1)CC(F)(F)C(=O)N2C]

### Step 1. Synthesis of tert-butyl (1-(2-(4-(4-((2,6-dioxopiperidin-3-yl)amino)-2-fluoro-5-methoxyphenyl)piperazin-1-yl)ethyl)piperidin-4-yl)carbamate (3)

It was synthesized by similar method described in step 3 of Example 23 to obtain tert-butyl (1-(2-(4-(4-((2,6-dioxopiperidin-3-yl)amino)-2-fluoro-5-methoxyphenyl)piperazin-1-yl)ethyl)piperidin-4-yl)carbamate (4.5 g, 8.00 mmol, 49.69% yield) as a blue solid. MS(M+H)⁺=563.3.

### Step 2. Synthesis of 3-((4-(4-(2-(4-aminopiperidin-1-yl)ethyl)piperazin-1-yl)-5-fluoro-2-methoxyphenyl)amino)piperidine-2,6-dione (4)

It was synthesized by similar method described in step 3 of Example 3 to obtain 3-((4-(4-(2-(4-aminopiperidin-1-yl)ethyl)piperazin-1-yl)-5-fluoro-2-methoxyphenyl)amino)piperidine-2,6-dione (2 g, 3.47 mmol, 97.59% yield, TFA salt) as brown oil. MS(M+H)⁺=463.3.

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2-fluoro-5-methoxyphenyl)piperazin-1-yl)ethyl)piperidin-4-yl)-2-fluoro-5-methoxybenz amide (Compound 49)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2-fluoro-5-m ethoxyphenyl)piperazin-1-yl)ethyl)piperidin-4-yl)-2-fluoro-5-methoxybenzamide (1.67 g, 1.81 mmol, 52.30% yield, 99% purity) as a blue solid. MS(M+H)⁺=910.4.

¹H NMR (400 MHz, DMSO-d₆) δ=10.85 (br s, 1H), 8.30 (s, 1H), 8.25 (d, J=13.3 Hz, 1H), 8.04 (s, 1H), 7.89 (br dd, J=2.8, 7.3 Hz, 1H), 7.20 (d, J=6.6 Hz, 1H), 6.60 (d, J=8.1 Hz, 1H), 6.55 (d, J=14.3 Hz, 1H), 5.09 (br d, J=6.4 Hz, 1H), 4.83 (br t, J=8.1 Hz, 1H), 4.32-4.18 (m, 1H), 4.08 (br t, J=13.9 Hz, 2H), 3.92 (s, 3H), 3.83-3.72 (m, 4H), 3.31 (br s, 2H), 2.93-2.76 (m, 7H), 2.59-2.51 (m, 6H), 2.49-2.42 (m, 4H), 2.16-1.90 (m, 6H), 1.84-1.70 (m, 4H), 1.67-1.51 (m, 6H).

### Example 50. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2-fluoro-5-methoxyphenyl)piperidin-4-yl)ethyl)piperidin-4-yl)-2-fluoro-5-methoxybenzamide (Compound 50)

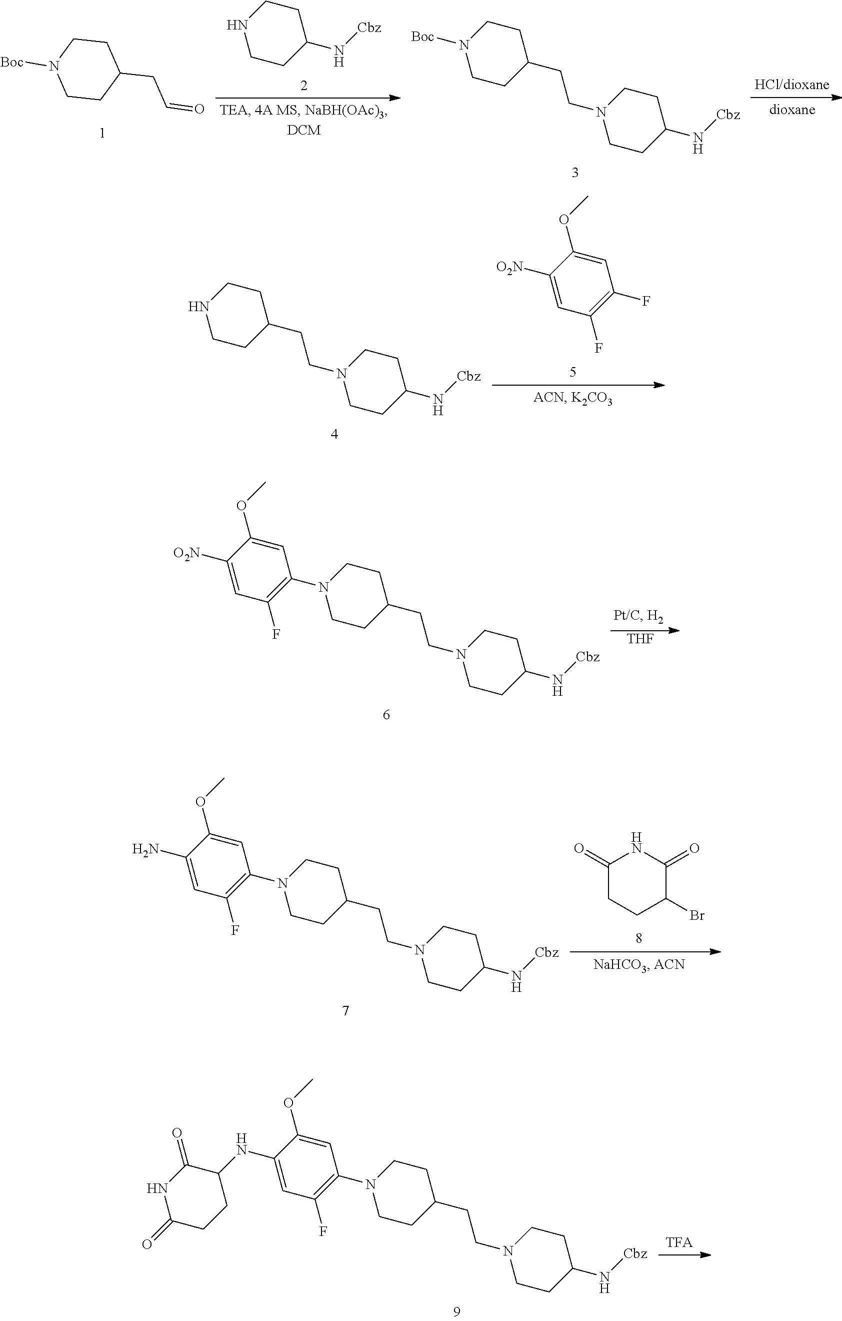

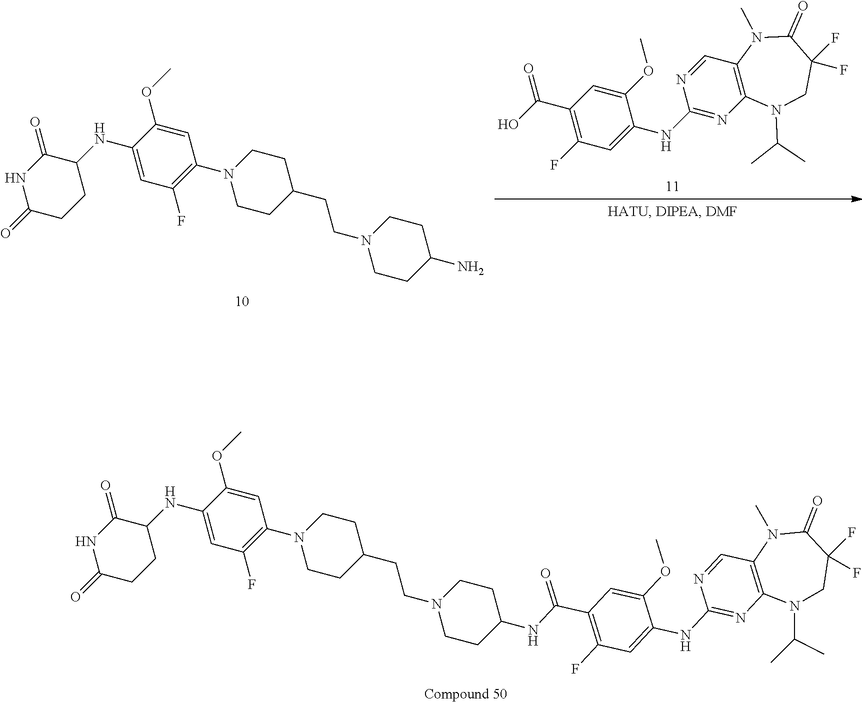 [SMILES: COc1cc(N2CCC(CCN3CCC(N)CC3)CC2)c(F)cc1NC1CCC(=O)NC1=O ; COc1cc(C(=O)O)c(F)cc1Nc1ncc2c(n1)N(C(C)C)CC(F)(F)C(=O)N2C ; COc1cc(C(=O)NC2CCN(CCC3CCN(c4cc(OC)c(NC5CCC(=O)NC5=O)cc4F)CC3)CC2)c(F)cc1Nc1ncc2c(n1)N(C(C)C)CC(F)(F)C(=O)N2C]

### Step 1. Synthesis of tert-butyl 4-(2-(4-(((benzyloxy)carbonyl)amino)piperidin-1-yl)ethyl)piperidine-1-carboxylate (3)

It was synthesized by similar method described in step 6 of Example 1 to obtain tert-butyl 4-(2-(4-(((benzyloxy)carbonyl)amino)piperidin-1-yl)ethyl)piperidine-1-carboxylate (7.7 g, 16.93 mmol, 81.90% yield, 98% purity) as a white solid. MS(M+H)⁺=446.3.

### Step 2. Synthesis of benzyl (1-(2-(piperidin-4-yl)ethyl)piperidin-4-yl)carbamate (4)

It was synthesized by similar method described in step 5 of Example 1 to obtain benzyl (1-(2-(piperidin-4-yl)ethyl)piperidin-4-yl)carbamate (6.5 g, crude, HCl) as a white solid. MS(M+H)⁺=346.1.

¹H NMR (400 MHz, CD₃OD) δ=7.44-7.19 (m, 5H), 5.22-4.96 (m, 2H), 3.73-3.62 (m, 4H), 3.47-3.36 (m, 2H), 3.25-3.14 (m, 2H), 3.14-2.89 (m, 4H), 2.19-1.93 (m, 4H), 1.90-1.66 (m, 4H), 1.56-1.37 (m, 2H).

### Step 3. Synthesis of benzyl (1-(2-(1-(2-fluoro-5-methoxy-4-nitrophenyl)piperidin-4-yl)ethyl)piperidin-4-yl)carbamate (6)

It was synthesized by similar method described in step 2 of Example 7 to obtain benzyl (1-(2-(1-(2-fluoro-5-methoxy-4-nitrophenyl)piperidin-4-yl)ethyl)piperidin-4-yl)carbamate (3 g, 5.83 mmol, 34.26% yield, 100% purity) as a yellow solid. MS(M+H)⁺=515.3.

### Step 4. Synthesis of benzyl (1-(2-(1-(4-amino-2-fluoro-5-methoxyphenyl)piperidin-4-yl)ethyl)piperidin-4-yl)carbamate (7)

It was synthesized by similar method described in step 1 of Example 35 to obtain benzyl (1-(2-(1-(4-amino-2-fluoro-5-methoxyphenyl)piperidin-4-yl)ethyl)piperidin-4-yl)carba mate (5.6 g, 10.75 mmol, 80.15% yield, 93% purity) as a brown solid. MS(M+H)⁺=485.2.

### Step 5. Synthesis of benzyl (1-(2-(1-(4-((2,6-dioxopiperidin-3-yl)amino)-2-fluoro-5-methoxyphenyl)piperidin-4-yl)ethyl)piperidin-4-yl)carbamate (9)

It was synthesized by similar method described in step 5 of Example 22 to obtain benzyl (1-(2-(1-(4-((2,6-dioxopiperidin-3-yl)amino)-2-fluoro-5-methoxyphenyl)piperidin-4-yl)ethyl)piperidin-4-yl)carbamate (3 g, 4.63 mmol, 74.84% yield, 92% purity) as a brown solid. MS(M+H)⁺=596.2.

### Step 6. Synthesis of 3-((4-(4-(2-(4-aminopiperidin-1-yl)ethyl)piperidin-1-yl)-5-fluoro-2-methoxyphenyl)amino)piperidine-2,6-dione (10)

It was synthesized by similar method described in step 2 of Example 4 to obtain 3-((4-(4-(2-(4-aminopiperidin-1-yl)ethyl)piperidin-1-yl)-5-fluoro-2-methoxyphenyl)amino)piperidine-2,6-dione (2.5 g, crude, TFA) as brown oil. MS(M+H)⁺=462.3.

### Step 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2-fluoro-5-methoxyphenyl)piperidin-4-yl)ethyl)piperidin-4-yl)-2-fluoro-5-methoxybenzamide (Compound 50)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2-fluoro-5-methoxyphenyl)piperidin-4-yl)ethyl)piperidin-4-yl)-2-fluoro-5-methoxybenzamide (507.3 mg, 561.92 μmol, 41.34% yield, 97.8% purity) as a purple solid. MS(M+H)⁺=883.4.

¹H NMR (400 MHz, DMSO-d₆) δ=10.85 (s, 1H), 8.34-8.24 (m, 2H), 7.97 (s, 1H), 7.93-7.80 (m, 1H), 7.20 (d, J=6.8 Hz, 1H), 6.60 (d, J=8.1 Hz, 1H), 6.53 (d, J=14.3 Hz, 1H), 5.08 (d, J=6.8 Hz, 1H), 4.95-4.85 (m, 1H), 4.29-4.20 (m, 1H), 4.07 (t, J=13.6 Hz, 2H), 3.92 (s, 3H), 3.78 (s, 3H), 3.34 (s, 3H), 3.18-3.11 (m, 2H), 2.86-2.76 (m, 2H), 2.66-2.53 (m, 4H), 2.40-2.26 (m, 2H), 2.16-2.08 (m, 1H), 2.04-1.89 (m, 3H), 1.85-1.72 (m, 4H), 1.63-1.51 (m, 2H), 1.44-1.24 (m, 12H).

### Example 51. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2-fluoro-5-methoxyphenyl)piperidin-4-yl)methyl)-7-azaspiro[3.5]nonan-2-yl)-3-methoxybenzamide (Compound 51)

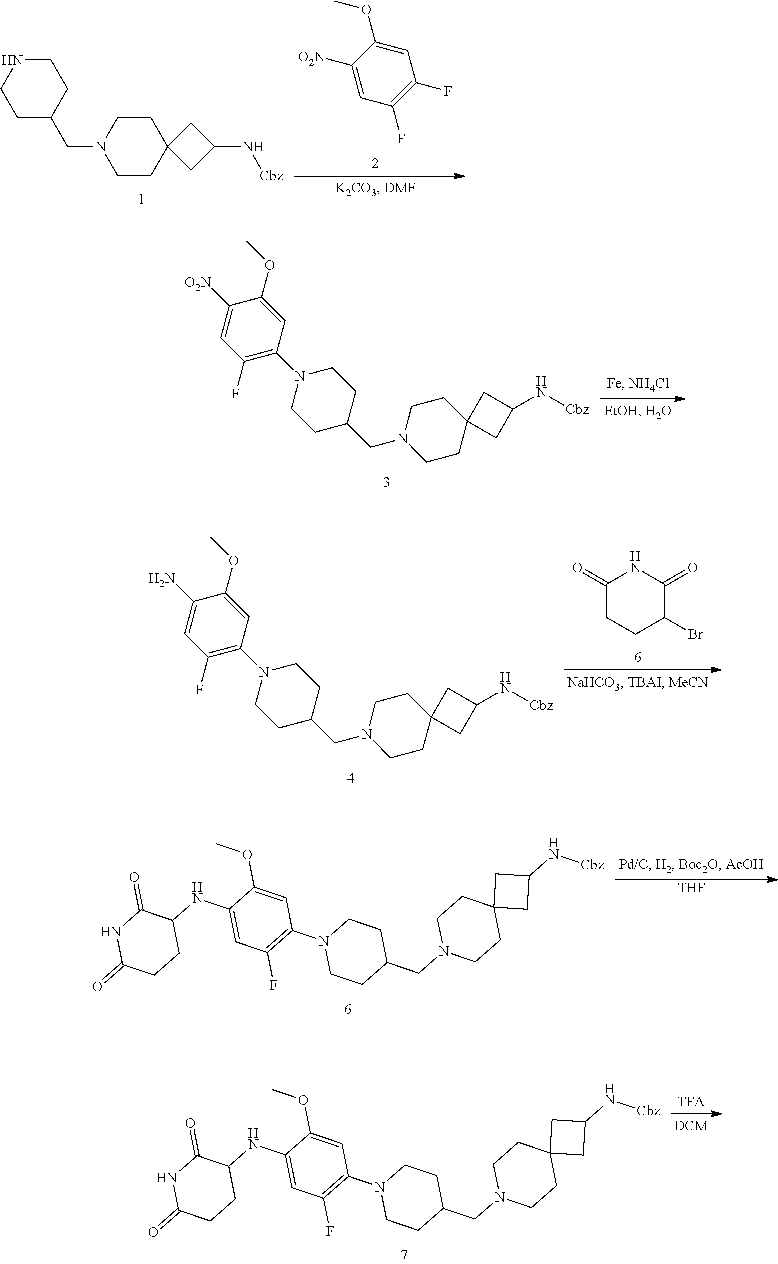

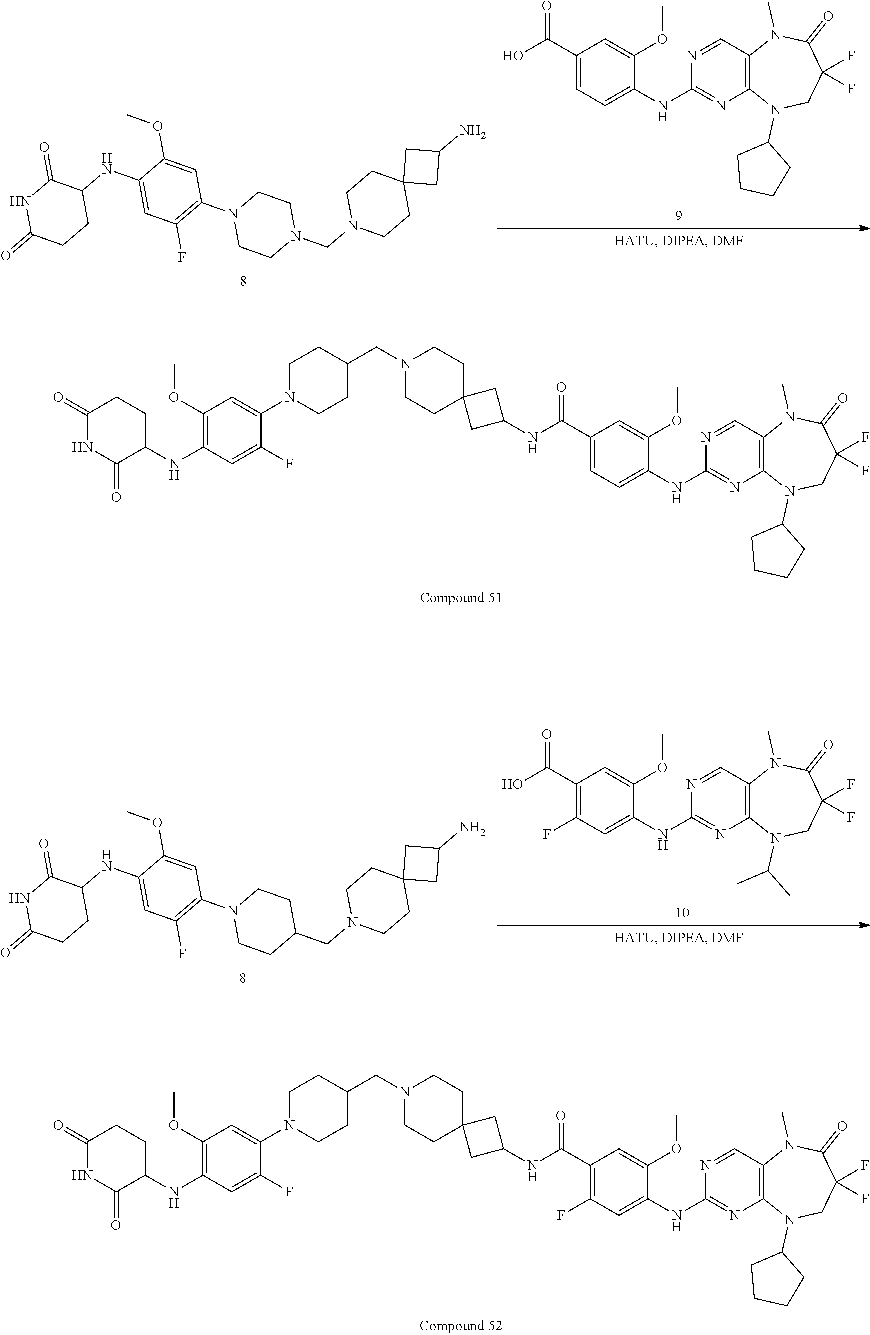 [SMILES: COc1cc(N2CCN(CN3CCC4(CC3)CC(N)C4)CC2)c(F)cc1NC1CCC(=O)NC1=O ; COc1cc(C(=O)O)ccc1Nc1ncc2c(n1)N(C1CCCC1)CC(F)(F)C(=O)N2C ; COc1cc(C(=O)NC2CC3(CCN(CC4CCN(c5cc(OC)c(NC6CCC(=O)NC6=O)cc5F)CC4)CC3)C2)ccc1Nc1ncc2c(n1)N(C1CCCC1)CC(F)(F)C(=O)N2C ; COc1cc(N2CCC(CN3CCC4(CC3)CC(N)C4)CC2)c(F)cc1NC1CCC(=O)NC1=O ; COc1cc(C(=O)O)c(F)cc1Nc1ncc2c(n1)N(C(C)C)CC(F)(F)C(=O)N2C ; COc1cc(C(=O)NC2CC3(CCN(CC4CCN(c5cc(OC)c(NC6CCC(=O)NC6=O)cc5F)CC4)CC3)C2)c(F)cc1Nc1ncc2c(n1)N(C1CCCC1)CC(F)(F)C(=O)N2C]

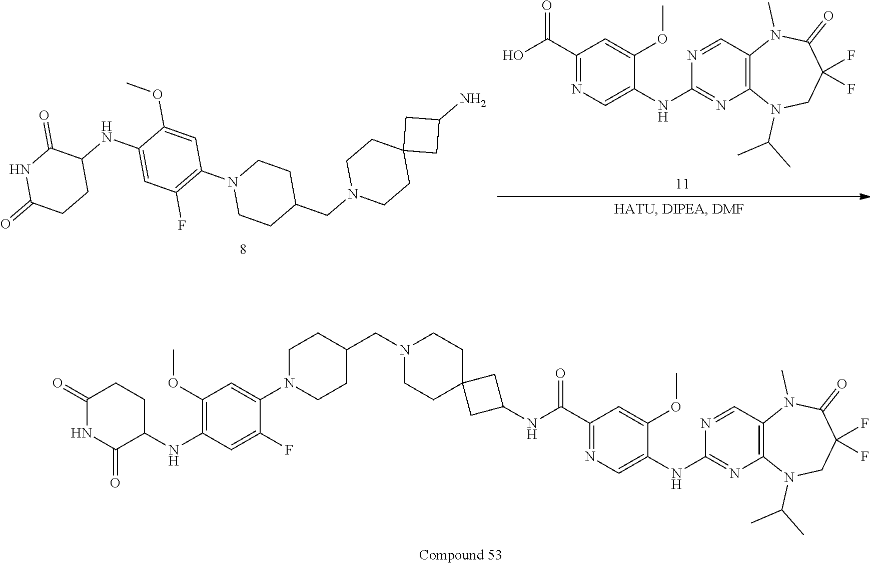 [SMILES: COc1cc(N2CCC(CN3CCC4(CC3)CC(N)C4)CC2)c(F)cc1NC1CCC(=O)NC1=O ; COc1cc(C(=O)O)ncc1Nc1ncc2c(n1)N(C(C)C)CC(F)(F)C(=O)N2C ; COc1cc(C(=O)NC2CC3(CCN(CC4CCN(c5cc(OC)c(NC6CCC(=O)NC6=O)cc5F)CC4)CC3)C2)ncc1Nc1ncc2c(n1)N(C(C)C)CC(F)(F)C(=O)N2C]

### Step 1. Synthesis of benzyl (7-((1-(2-fluoro-5-methoxy-4-nitrophenyl)piperidin-4-yl)methyl)-7-azaspiro[3.5]nonan-2-yl)carbamate (3)

It was synthesized by similar method described in step 4 of Example 30 to obtain benzyl (7-((1-(2-fluoro-5-methoxy-4-nitrophenyl)piperidin-4-yl)methyl)-7-azaspiro[3.5]nonan-2-yl)carbamate (11.26 g, 19.16 mmol, 90.33% yield, 92% purity) as a yellow oil. MS(M+H)⁺=541.4.

### Step 2. Synthesis of benzyl (7-((1-(4-amino-2-fluoro-5-methoxyphenyl)piperidin-4-yl)methyl)-7-azaspiro[3.5]nonan-2-yl)carbamate (4)

It was synthesized by similar method described in step 4 of Example 22 to obtain benzyl (7-((1-(4-amino-2-fluoro-5-methoxyphenyl)piperidin-4-yl)methyl)-7-azaspiro[3.5]nonan-2-yl)carbamate (9.14 g, crude) as a red oil. MS(M+H)⁺=511.3.

### Step 3. Synthesis of benzyl (7-((1-(4-((2,6-dioxopiperidin-3-yl)amino)-2-fluoro-5-methoxyphenyl)piperidin-4-yl)methyl)-7-azaspiro[3.5]nonan-2-yl)carbamate (6)

It was synthesized by similar method described in step 5 of Example 22, addition TBAI (1.87 g, 5.08 mmol) to obtain benzyl (7-((1-(4-((2,6-dioxopiperidin-3-yl)amino)-2-fluoro-5-methoxyphenyl)piperidin-4-yl) methyl)-7-azaspiro[3.5]nonan-2-yl)carbamate (1.8 g, 2.78 mmol, 16.43% yield, 96% purity) as a gray solid. MS(M+H)⁺=622.3.

### Step 4. Synthesis of tert-butyl (7-((1-(4-((2,6-dioxopiperidin-3-yl)amino)-2-fluoro-5-methoxyphenyl)piperidin-4-yl)methyl)-7-azaspiro[3.5]nonan-2-yl)carbamate (7)

To a solution of Pd/C (1 g, 10% purity) in THF (60 mL) was added a solution of Boc₂O (3.58 g, 16.41 mmol, 3.77 mL), AcOH (340 mg, 5.66 mmol, 324.12 μL) and benzyl (7-((1-(4-((2,6-dioxopiperidin-3-yl)amino)-2-fluoro-5-methoxyphenyl)piperidin-4-yl) methyl)-7-azaspiro[3.5]nonan-2-yl)carbamate (6.8 g, 10.94 mmol) in THF (100 mL) under N₂ atmosphere. The mixture was stirred at 25° C. under H₂ (45 psi) atmosphere for 28 h. LCMS showed the desired mass was detected. The mixture was filtered and the filter cake was washed with THF (500 mL). The filtrate was concentrated under reduced pressure (25° C.). The residue was diluted with EtOAc (100 mL) and saturated aq. NaHCO₃ (50 mL), extracted with EtOAc (50 mL×3), the combined organic layer was washed with saturated aq. NaHCO₃ (50 mL) and water (50 mL), dried over Na₂SO 4 and filtered. The filtrate was concentrated under reduced pressure and then diluted with MTBE/EtOH=4/1 (65 mL) and the mixture was stirred at 25° C. for 14 h. The filter cake was collected and triturated with MTBE/EtOH=4/1 (30 mL, twice) and then filtered. The filter cake was collected and dried to afford tert-butyl (7-((1-(4-((2,6-dioxopiperidin-3-yl)amino)-2-fluoro-5-methoxyphenyl)piperidin-4-yl) methyl)-7-azaspiro[3.5]nonan-2-yl)carbamate (4.74 g, 8.07 mmol, 73.74% yield) as a blue solid. The filtrate was concentrated under reduced pressure and triturated with MTBE/EtOH=4/1 (20 mL) at 25° C. for 14 h. The mixture was filtered and the filter cake was washed with MTBE/EtOH=4/1 (10 mL) and the filter cake was collected to afford tert-butyl (7-((1-(4-((2,6-dioxopiperidin-3-yl)amino)-2-fluoro-5-methoxyphenyl)piperidin-4-yl) methyl)-7-azaspiro[3.5]nonan-2-yl)carbamate (680 mg, 1.16 mmol, 10.58% yield, 100% purity) as a blue solid. MS(M+H)⁺=588.3.

### Step 5. Synthesis of 3-((4-(4-((2-amino-7-azaspiro[3.5]nonan-7-yl)methyl)piperidin-1-yl)-5-fluoro-2-methoxyphenyl)amino)piperidine-2,6-dione (8)

It was synthesized by similar method described in step 6 of Example 3 to obtain 3-((4-(4-((2-amino-7-azaspiro[3.5]nonan-7-yl)methyl)piperidin-1-yl)-5-fluoro-2-methoxyphenyl)amino)piperidine-2,6-dione (1.83 g, crude, TFA) as a yellow oil. MS(M+H)⁺=488.2.

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2-fluoro-5-methoxyphenyl)piperidin-4-yl)methyl)-7-azaspiro[3.5]nonan-2-yl)-3-methoxybenzamide (Compound 51)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2-fluoro-5-methoxyphenyl)piperidin-4-yl)methyl)-7-azaspiro[3.5]nonan-2-yl)-3-methoxybenzamide (1.18 g, 1.27 mmol, 51.66% yield, 98.7% purity) as a yellow solid. MS(M+H)⁺=917.6.

¹H NMR (400 MHz, DMSO-d₆) δ=10.84 (s, 1H), 8.42 (d, J=7.3 Hz, 1H), 8.29-8.25 (m, 2H), 7.96 (s, 1H), 7.51-7.46 (m, 2H), 6.60 (d, J=8.2 Hz, 1H), 6.52 (d, J=14.2 Hz, 1H), 5.07 (d, J=7.0 Hz, 1H), 4.82-4.71 (m, 1H), 4.44-4.35 (m, 1H), 4.29-4.20 (m, 1H), 4.04 (t, J=14.0 Hz, 2H), 3.94 (s, 3H), 3.77 (s, 3H), 3.33 (s, 3H), 3.17-3.10 (m, 2H), 2.84-2.75 (m, 1H), 2.64-2.54 (m, 3H), 2.35-2.09 (m, 9H), 1.99-1.88 (m, 3H), 1.85-1.78 (m, 2H), 1.77-1.67 (m, 4H), 1.65-1.52 (m, 9H), 1.29-1.17 (m, 2H).

### Example 52. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2-fluoro-5-methoxyphenyl)piperidin-4-yl)methyl)-7-azaspiro[3.5]nonan-2-yl)-2-fluoro-5-methoxybenzamide (Compound 52)

It was synthesized by similar method described in Example 51 to obtain 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2-fluoro-5-meth oxyphenyl)piperidin-4-yl)methyl)-7-azaspiro[3.5]nonan-2-yl)-2-fluoro-5-methoxybenz amide (905.6 mg, 968.38 mol, 38.68% yield, 97.2% purity) as a yellow solid. MS(M+H)⁺=909.5.

¹H NMR (400 MHz, DMSO-d₆) δ=10.90-10.77 (m, 1H), 8.31-8.16 (m, 3H), 7.96 (s, 1H), 7.19 (d, J=6.7 Hz, 1H), 6.59 (d, J=8.2 Hz, 1H), 6.52 (d, J=14.3 Hz, 1H), 5.06 (d, J=6.2 Hz, 1H), 4.94-4.85 (m, 1H), 4.43-4.31 (m, 1H), 4.29-4.21 (m, 1H), 4.11-4.01 (m, 2H), 3.91 (s, 3H), 3.77 (s, 3H), 3.31-3.27 (m, 3H), 3.17-3.09 (m, 2H), 2.86-2.75 (m, 1H), 2.64-2.53 (m, 3H), 2.36-2.08 (m, 9H), 1.97-1.84 (m, 1H), 1.83-1.71 (m, 4H), 1.66-1.49 (m, 5H), 1.33-1.15 (m, 8H).

### Example 53. Synthesis of 5-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2-fluoro-5-methoxyphenyl)piperidin-4-yl)methyl)-7-azaspiro[3.5]nonan-2-yl)-4-methoxypicolinamide (Compound 53)

To a solution of 5-((7,7-difluoro-9-isopropyl-5-methyl-6-oxo-6,7,8,9-tetrahydro-5H-pyrimido[4,5-b][1,4]diazepin-2-yl)amino)-4-methoxypicolinic acid (1.5 g, 3.55 mmol) and HATU (1.62 g, 4.26 mmol) in DMF (20 mL) was added DIPEA (4.59 g, 35.51 mmol, 6.19 mL) and the mixture was stirred at 25° C. for 15 min. Then a solution of 3-((4-(4-((2-amino-7-azaspiro[3.5]nonan-7-yl)methyl)piperidin-1-yl)-5-fluoro-2-methoxyphenyl)amino)piperidine-2,6-dione (2.4 g, 3.99 mmol, TFA) in DMF (20 mL) was added and the mixture was stirred at 25° C. for 3 h. LCMS showed 53% the desired mass and 27% 3-((4-(4-((2-amino-7-azaspiro[3.5]nonan-7-yl)methyl)piperidin-1-yl)-5-fluoro-2-methoxyphenyl)amino)piperidine-2,6-dione remained. Then a solution of 5-((7,7-difluoro-9-isopropyl-5-methyl-6-oxo-6,7,8,9-tetrahydro-5H-pyrimido[4,5-b][1,4]diazepin-2-yl)amino)-4-methoxypicolinic acid (450 mg, 1.07 mmol), HATU (419 mg, 1.10 mmol) and DIPEA (371.00 mg, 2.87 mmol, 0.5 mL) in DMF (5 mL) was added and the mixture was stirred at 25° C. for 1 h. LCMS showed 77% the desired mass. The mixture was diluted with EtOAc (50 mL) and saturated aq. NaHCO₃ (100 mL) and then extracted with EtOAc (20 mL×3), the combined organic layer was washed with brine (30 mL×2), dried over Na₂SO₄ and filtered. The filtrate was concentrated under reduced pressure which was purified by flash silica gel chromatography (20 g SepaFlash® Silica Flash Column, Eluent of 7-13% MeOH/DCM gradient @ 100 mL/min), the product was diluted with 1 N HCl (60 mL) and the mixture was stirred at 25° C. for 10 min. The mixture was washed with EtOAc (30 mL×3), the aqueous phase was slowly added to saturated aq. NaHCO₃ (200 mL). Then the mixture was filtered and the filter cake was washed with deionized water (30 mL×3). The filter cake was collected and the solvent was removed under reduced pressure to afford 5-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2-fluoro-5-meth oxyphenyl)piperidin-4-yl)methyl)-7-azaspiro[3.5]nonan-2-yl)-4-methoxypicolinamide (2.08 g, 2.30 mmol, 64.74% yield, 98.6% purity) as a white solid. MS(M+H)⁺=892.6.

¹H NMR (400 MHz, DMSO-d₆) δ=10.84 (s, 1H), 9.12 (s, 1H), 8.83-8.71 (m, 1H), 8.23-8.14 (m, 2H), 7.64 (s, 1H), 6.59 (d, J=8.1 Hz, 1H), 6.53 (d, J=14.1 Hz, 1H), 5.11-5.04 (m, 1H), 4.85-4.75 (m, 1H), 4.49-4.35 (m, 1H), 4.30-4.21 (m, 1H), 4.08-3.95 (m, 5H), 3.78 (s, 3H), 3.32 (s, 3H), 3.18-3.10 (m, 2H), 2.86-2.73 (m, 1H), 2.66-2.55 (m, 4H), 2.30-2.05 (m, 7H), 1.99-1.70 (m, 7H), 1.65-1.48 (m, 4H), 1.29-1.14 (m, 8H).

### Example 54. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-3-methoxyphenyl)piperazin-1-yl)ethyl)piperidin-1-yl)-3-methoxybenzamide (Compound 54)

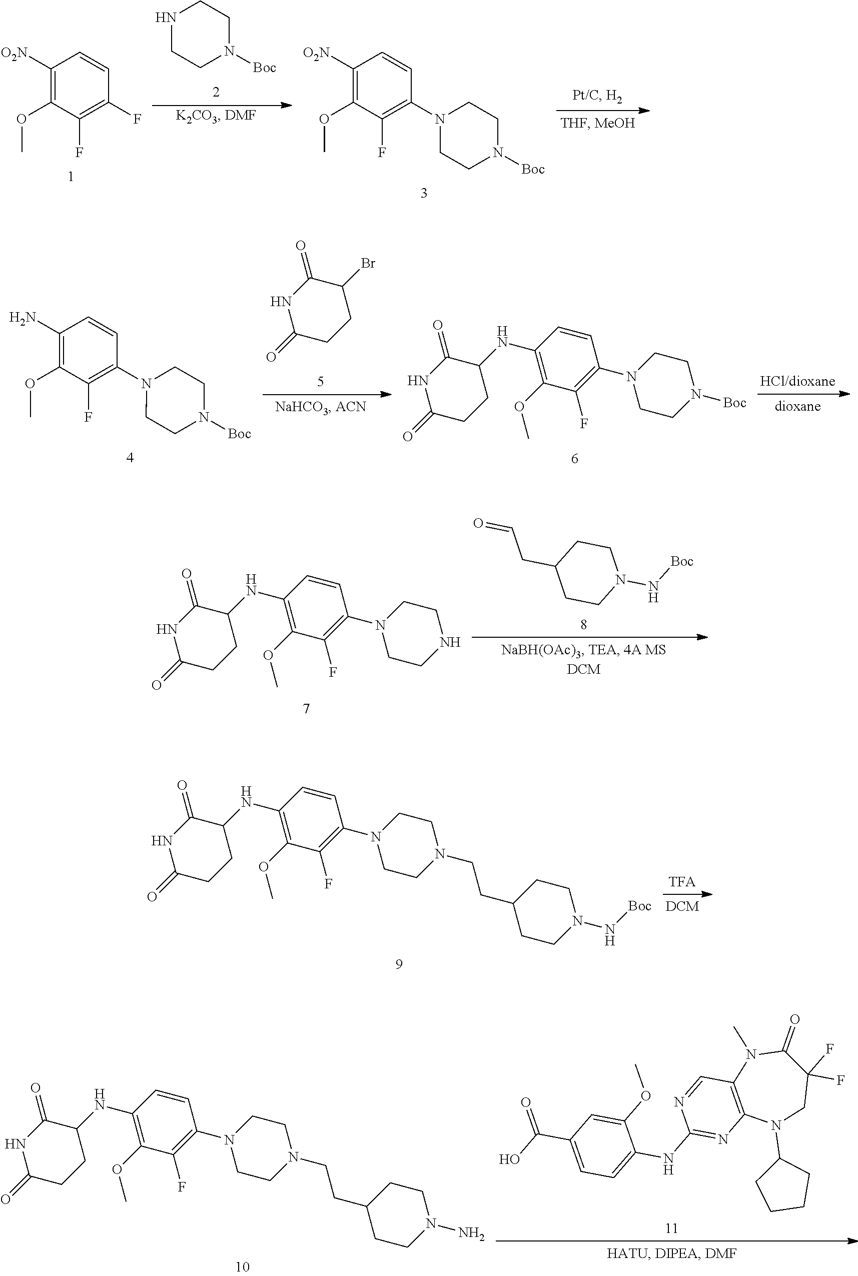

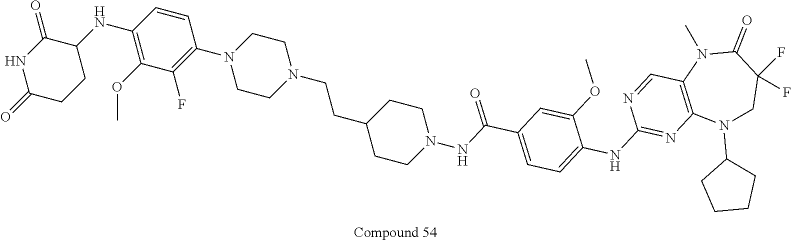 [SMILES: COc1cc(C(=O)NN2CCC(CCN3CCN(c4ccc(NC5CCC(=O)NC5=O)c(OC)c4F)CC3)CC2)ccc1Nc1ncc2c(n1)N(C1CCCC1)CC(F)(F)C(=O)N2C]

### Step 1. Synthesis of tert-butyl 4-(2-fluoro-3-methoxy-4-nitrophenyl)piperazine-1-carboxylate (3)

It was synthesized by similar method described in step 4 of Example 30 to obtain tert-butyl 4-(2-fluoro-3-methoxy-4-nitrophenyl)piperazine-1-carboxylate (16.3 g, 43.58 mmol, 82.41% yield, 95% purity) as an yellow solid. MS(M-t-Bu+H)⁺=300.1.

### Step 2. Synthesis of tert-butyl 4-(4-amino-2-fluoro-3-methoxyphenyl)piperazine-1-carboxylate (4)

It was synthesized by similar method described in step 1 of Example 35, addition MeOH (160 mL) to obtain tert-butyl 4-(4-amino-2-fluoro-3-methoxyphenyl)piperazine-1-carboxylate (14 g, 43.03 mmol, 95.56% yield) as a red solid. MS(M+H)⁺=326.1.

### Step 3. Synthesis of tert-butyl 4-(4-((2,6-dioxopiperidin-3-yl)amino)-2-fluoro-3-methoxyphenyl)piperazine-1-carboxylate (6)

It was synthesized by similar method described in step 5 of Example 22 to obtain tert-butyl 4-(4-((2,6-dioxopiperidin-3-yl)amino)-2-fluoro-3-methoxyphenyl)piperazine-1-carboxylate (5.3 g, 12.02 mmol, 78.23% yield, 99% purity) as a blue solid. MS(M+H)⁺=437.2.

### Step 4. Synthesis of 3-((3-fluoro-2-methoxy-4-(piperazin-1-yl)phenyl)amino)piperidine-2,6-dione (7)

It was synthesized by similar method described in step 5 of Example 1 to obtain 3-((3-fluoro-2-methoxy-4-(piperazin-1-yl)phenyl)amino)piperidine-2,6-dione (5 g, crude, HCl) as a blue solid. MS(M+H)⁺=337.1.

### Step 5. Synthesis of tert-butyl (4-(2-(4-(4-((2,6-dioxopiperidin-3-yl)amino)-2-fluoro-3-methoxyphenyl)piperazin-1-yl)ethyl)piperidin-1-yl)carbamate (9)

It was synthesized by similar method described in step 6 of Example 1 to obtain tert-butyl (4-(2-(4-(4-((2,6-dioxopiperidin-3-yl)amino)-2-fluoro-3-methoxyphenyl)piperazin-1-yl)ethyl)piperidin-1-yl)carbamate (1.4 g, 2.39 mmol, 44.53% yield, 96% purity) as a blue solid. MS(M+H)⁺=563.3.

### Step 6. Synthesis of 3-((4-(4-(2-(1-aminopiperidin-4-yl)ethyl)piperazin-1-yl)-3-fluoro-2-methoxyphenyl)amino)piperidine-2,6-dione (10)

It was synthesized by similar method described in step 6 of Example 3 to obtain 3-((4-(4-(2-(1-aminopiperidin-4-yl)ethyl)piperazin-1-yl)-3-fluoro-2-methoxyphenyl)amino)piperidine-2,6-dione (1.3 g, 2.25 mmol, 97.59% yield, TFA) as brown oil. MS(M+H)⁺=463.4.

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-3-methoxyphenyl)piperazin-1-yl)ethyl)piperidin-1-yl)-3-methoxybenzamide (Compound 54)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-3-m ethoxyphenyl)piperazin-1-yl)ethyl)piperidin-1-yl)-3-methoxybenzamide (481.7 mg, 524.37 μmol, 23.26% yield, 97.1% purity) as a white solid. MS(M+H)⁺=892.4.

¹H NMR (400 MHz, DMSO-d₆) δ=10.85 (s, 1H), 9.29 (s, 1H), 8.31-8.19 (m, 2H), 7.97 (s, 1H), 7.49-7.37 (m, 2H), 6.64 (t, J=8.3 Hz, 1H), 6.46 (d, J=8.5 Hz, 1H), 5.26 (s, 1H), 4.82-4.72 (m, 1H), 4.36-4.24 (m, 1H), 4.05 (t, J=13.9 Hz, 2H), 3.93 (s, 3H), 3.78 (s, 3H), 3.33 (s, 3H), 3.09-2.97 (m, 3H), 2.95-2.86 (m, 3H), 2.84-2.72 (m, 4H), 2.61-2.52 (m, 3H), 2.49-2.25 (m, 3H), 2.16-2.07 (m, 1H), 2.05-1.87 (m, 3H), 1.79-1.68 (m, 4H), 1.65-1.55 (m, 4H), 1.53-1.39 (m, 2H), 1.38-1.23 (m, 3H).

### Example 55. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-3-fluoro-2-methoxyphenyl)piperazin-1-yl)ethyl)piperidin-1-yl)-3-methoxybenzamide (Compound 55)

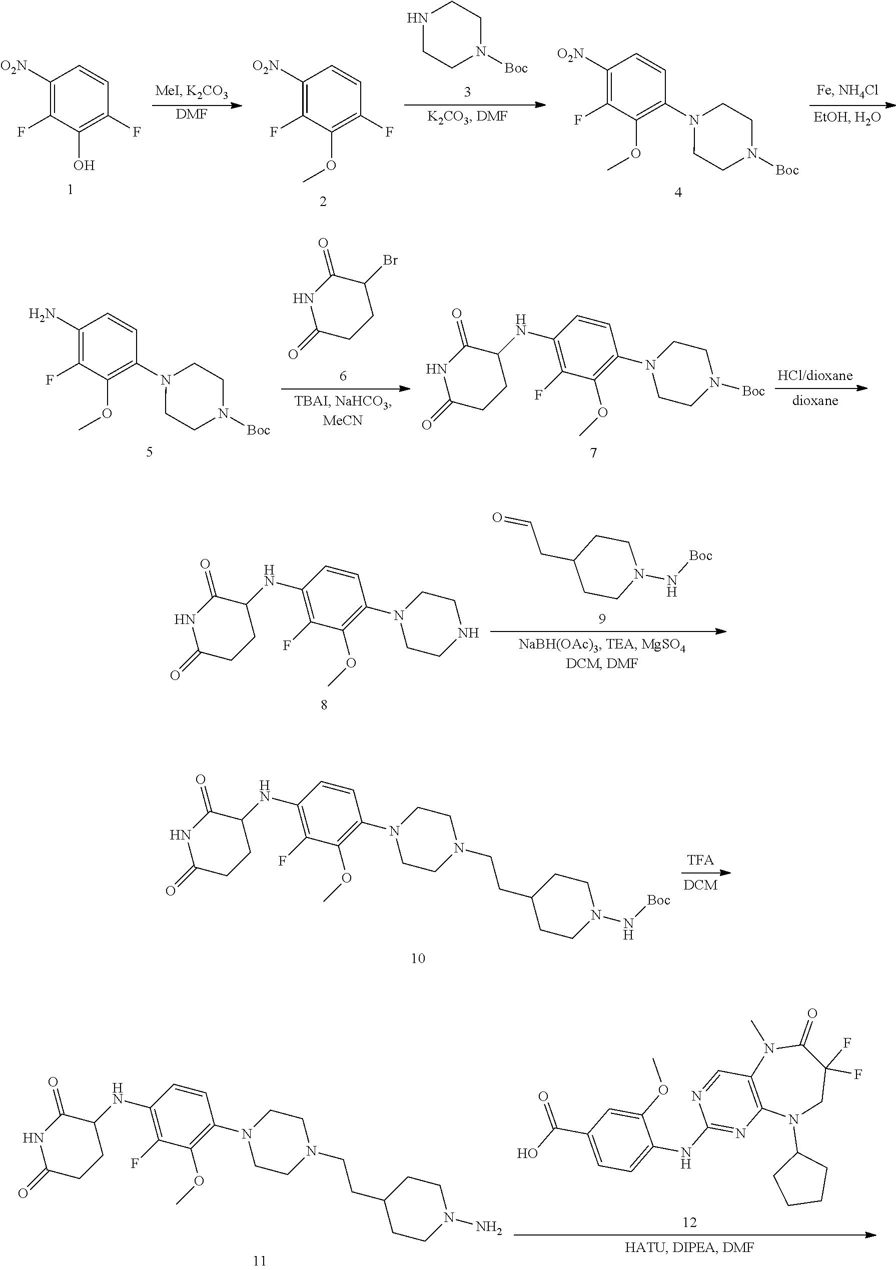

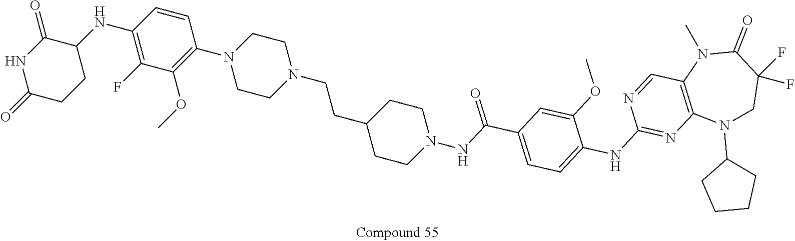 [SMILES: COc1cc(C(=O)NN2CCC(CCN3CCN(c4ccc(NC5CCC(=O)NC5=O)c(F)c4OC)CC3)CC2)ccc1Nc1ncc2c(n1)N(C1CCCC1)CC(F)(F)C(=O)N2C]

### Step 1. Synthesis of 1,3-difluoro-2-methoxy-4-nitrobenzene (2)

To a solution of 2,6-difluoro-3-nitrophenol (5 g, 28.56 mmol) in DMF (70 mL) were added K₂CO₃ (7.89 g, 57.11 mmol) and Mel (6.16 g, 43.37 mmol, 2.7 mL) and the mixture was stirred at 25° C. for 14 h. TLC (Petroleum ether:EtOAc=1:0) showed the starting material was consumed and new spot was detected. The mixture was diluted with EtOAc (50 mL) and brine (100 mL) and then extracted with EtOAc (20 mL×3), the combined organic layer was washed with brine (50 mL×2), dried over Na₂SO₄ and filtered. The filtrate was concentrated under reduced pressure to afford 1,3-difluoro-2-methoxy-4-nitrobenzene (6 g, crude) as a yellow oil.

### Step 2. Synthesis of tert-butyl 4-(3-fluoro-2-methoxy-4-nitrophenyl)piperazine-1-carboxylate (4)

To a solution of 1,3-difluoro-2-methoxy-4-nitrobenzene (6 g, 31.73 mmol) and K₂CO 3 (5.26 g, 38.07 mmol) in DMF (200 mL) was added tert-butyl piperazine-1-carboxylate (5.4 g, 28.99 mmol) and the mixture was stirred at 100° C. for 1 h. LCMS showed the desired mass. The mixture was diluted with water (200 mL) and extracted with EtOAc (100 mL×3), the combined organic layer was washed with brine (50 mL×3), dried over Na₂SO₄ and filtered. The filtrate was concentrated under reduced pressure. The residue was triturated with MTBE (50 mL) at 25° C. for 14 h. The mixture was filtered and the filter cake was washed with MTBE (10 mL). The filter cake was collected and the solvent was removed under reduced pressure to afford 4.74 g of product 4.74 g with 97% purity. The filtrate was concentrated under reduced pressure and then purified by flash silica gel chromatography (120 g SepaFlash® Silica Flash Column, Eluent of 10-20% EtOAc/Petroleum ether gradient @ 100 mL/min) to afford 3.46 g of product. The two batches of products were combined to afford tert-butyl 4-(3-fluoro-2-methoxy-4-nitrophenyl)piperazine-1-carboxylate (8.2 g, 22.38 mmol, 70.55% yield, 97% purity) as a yellow solid. MS(M+H)⁺=356.2.

¹H NMR (400 MHz, DMSO-d₆) δ=7.85 (dd, J=8.5, 9.4 Hz, 1H), 6.88 (dd, J=1.5, 9.5 Hz, 1H), 3.83 (s, 3H), 3.53-3.46 (m, 4H), 3.31-3.27 (m, 4H), 1.42 (s, 9H).

### Step 3. Synthesis of tert-butyl 4-(4-amino-3-fluoro-2-methoxyphenyl)piperazine-1-carboxylate (5)

To a solution of tert-butyl 4-(3-fluoro-2-methoxy-4-nitrophenyl)piperazine-1-carboxylate (8.2 g, 23.08 mmol) and NH₄Cl (3.70 g, 69.22 mmol) in EtOH (150 mL) and H₂O (50 mL) was added Fe (3.87 g, 69.23 mmol) and the mixture was stirred at 80° C. for 1 h. LCMS showed 84% of the desired mass. The mixture (80° C.) was filtered and the filter cake was washed with EtOAc (200 mL). The filtrate was concentrated under reduced pressure and then adjusted the pH=8 with saturated aq. NaHCO₃. The mixture was extracted with EtOAc (50 mL×3), the combined organic layer was washed with water (50 mL), dried over Na₂SO₄ and filtered. The filtrate was concentrated under reduced pressure to afford tert-butyl 4-(4-amino-3-fluoro-2-methoxyphenyl)piperazine-1-carboxylate (7.6 g, crude) as a yellow solid. MS(M+H)⁺=326.2.

### Step 4. Synthesis of tert-butyl 4-(4-((2,6-dioxopiperidin-3-yl)amino)-3-fluoro-2-methoxyphenyl)piperazine-1-carboxylate (7)

A solution of tert-butyl 4-(4-amino-3-fluoro-2-methoxyphenyl)piperazine-1-carboxylate (8.5 g, 26.12 mmol), 3-bromopiperidine-2,6-dione (10.03 g, 52.25 mmol), TBAI (964.92 mg, 2.61 mmol) and NaHCO₃ (8.78 g, 104.49 mmol, 4.07 mL) in MeCN (20 mL) was stirred at 80° C. for 28 h. LCMS showed 84% of the desired mass. The mixture was diluted with EtOAc (200 mL) and then filtered. The filter cake was washed with EtOAc (30 mL) and the filtrate was concentrated under reduced pressure. The residue was purified by flash silica gel chromatography (80 g SepaFlash® Silica Flash Column, Eluent of 40~80% EtOAc/Petroleum ether gradient @ 100 mL/min) to afford tert-butyl 4-(4-((2,6-dioxopiperidin-3-yl)amino)-3-fluoro-2-methoxyphenyl)piperazine-1-carboxylate (8.73 g, 17.40 mmol, 66.61% yield, 87% purity) as a blue solid. MS(M+H)⁺=437.3.

### Step 5. Synthesis of 3-((2-fluoro-3-methoxy-4-(piperazin-1-yl)phenyl)amino)piperidine-2,6-dione (8)

To a solution of tert-butyl 4-(4-((2,6-dioxopiperidin-3-yl)amino)-3-fluoro-2-methoxyphenyl)piperazine-1-carboxylate (4 g, 7.97 mmol) in DCM (50 mL) was added HCl/dioxane (2 M, 100 mL) and the mixture was stirred at 25° C. for 3 h. The mixture was concentrated under reduced pressure (25° C.) to afford 3-((2-fluoro-3-methoxy-4-(piperazin-1-yl)phenyl)amino)piperidine-2,6-dione (3.8 g, crude, HCl) as a blue solid. MS(M+H)⁺=337.2.

### Step 6. Synthesis of tert-butyl (4-(2-(4-(4-((2,6-dioxopiperidin-3-yl)amino)-3-fluoro-2-methoxyphenyl)piperazin-1-yl)ethyl)piperidin-1-yl)carbamate (10)

To a solution of 3-((2-fluoro-3-methoxy-4-(piperazin-1-yl)phenyl)amino)piperidine-2,6-dione (3.6 g, 9.66 mmol, HCl) in DMF (60 mL) and DCM (60 mL) were added TEA (3.14 g, 31.04 mmol, 4.32 mL) and MgSO₄ (2.32 g, 19.31 mmol) and followed by tert-butyl (4-(2-oxoethyl)piperidin-1-yl)carbamate (3.04 g, 12.55 mmol) and the mixture was stirred at 25° C. for 0.5 h. NaBH(OAc)₃ (4.09 g, 19.31 mmol) was added and the mixture was stirred at 25° C. for 14 h. tert-butyl (4-(2-oxoethyl)piperidin-1-yl)carbamate (1.1 g, 4.54 mmol) was added and the mixture was stirred at 25° C. for 1 h. LCMS showed 70% the desired mass. The mixture was diluted with water (100 mL) and extracted with EtOAc (30 mL×3), the combined organic layer was washed with brine (50 mL×2), dried over Na₂SO₄ and filtered. The filtrate was concentrated under reduced pressure. The residue was purified by flash silica gel chromatography (40 g SepaFlash® Silica Flash Column, Eluent of 10-20% MeOH/EtOAc gradient @ 100 mL/min) and repurified by reversed-phase HPLC (0.1% TFA condition, Column 330 g Flash Column Welch Ultimate XB_C₁₈ 20-40 μm; 120 ASolvent for sample dissolution; about 3.00 grams of sample dissolved in 100 mL of MeCN; Flow rate: 100 mL/min; Mobile phase: MeCN/H₂O; Gradient B %: 30-50% 45 min;% min Instrument ISCO), the eluent was lyophilized to afford tert-butyl (4-(2-(4-(4-((2,6-dioxopiperidin-3-yl)amino)-3-fluoro-2-methoxyphenyl)piperazin-1-yl)ethyl)piperidin-1-yl)carbamate (2.7 g, 3.83 mmol, 39.67% yield, 96% purity, TFA) as a blue solid. MS(M+H)⁺=563.4.

### Step 7. Synthesis of 3-((4-(4-(2-(1-aminopiperidin-4-yl)ethyl)piperazin-1-yl)-2-fluoro-3-methoxyphenyl)amino)piperidine-2,6-dione (11)

To a solution of tert-butyl (4-(2-(4-(4-((2,6-dioxopiperidin-3-yl)amino)-3-fluoro-2-methoxyphenyl)piperazin-1-yl)ethyl)piperidin-1-yl)carbamate (2.7 g, 4.80 mmol) in DCM (10 mL) was added TFA (8.29 g, 72.70 mmol, 5.4 mL) and the mixture was stirred at 25° C. for 4 h. LCMS showed 93% of the desired mass. The mixture was concentrated under reduced pressure to afford 3-((4-(4-(2-(1-aminopiperidin-4-yl)ethyl)piperazin-1-yl)-2-fluoro-3-methoxyphenyl)amino)piperidine-2,6-dione (2.78 g, crude, TFA) as a blue oil. MS(M+H)⁺=463.4.

### Step 8. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-3-fluoro-2-methoxyphenyl)piperazin-1-yl)ethyl)piperidin-1-yl)-3-methoxybenzamide (Compound 55)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (1.6 g, 3.58 mmol) and HATU (1.63 g, 4.29 mmol) in DMF (10 mL) was added DIPEA (4.62 g, 35.76 mmol, 6.23 mL) and the mixture was stirred at 15° C. for 0.5 h. Then a solution of 3-((4-(4-(2-(1-aminopiperidin-4-yl)ethyl)piperazin-1-yl)-2-fluoro-3-methoxyphenyl)amino)piperidine-2,6-dione (2.78 g, 4.83 mmol, TFA) in DMF (10 mL) was added at 0° C. and the mixture was stirred at 15° C. for 1 h. LCMS showed 55% of the desired mass. The mixture was diluted with water (10 mL) and saturated aq. NaHCO₃ (30 mL) and then extracted with EtOAc (30 mL×3), the combined organic layer was washed with brine (20 mL×3), dried over Na₂SO₄ and filtered. The filtrate was concentrated under reduced pressure to afford 3 g of the product. The product (1 g) was purified by prep-HPLC (column: Phenomenex luna C18 150*25 mm*10 um; mobile phase: [water (TFA)-ACN]; gradient: 15%-45% B over 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-3-fluoro-2-m ethoxyphenyl)piperazin-1-yl)ethyl)piperidin-1-yl)-3-methoxybenzamide (312.40 mg, 273.34 μmol, 7.64% yield, 98% purity, 2TFA) as a yellow solid. MS(M+H)⁺=892.6.

¹H NMR (400 MHz, DMSO-d₆) δ=10.81 (s, 1H), 9.79-9.31 (m, 2H), 8.30-8.12 (m, 3H), 7.53-7.38 (m, 2H), 6.66-6.60 (m, 1H), 6.55-6.47 (m, 1H), 4.84-4.75 (m, 1H), 4.37-4.30 (m, 1H), 4.13-4.04 (m, 2H), 3.93 (s, 3H), 3.86 (s, 3H), 3.61-3.55 (m, 2H), 3.45-3.37 (m, 2H), 3.33 (s, 3H), 3.26-3.09 (m, 6H), 2.95-2.83 (m, 4H), 2.80-2.70 (m, 1H), 2.62-2.56 (m, 1H), 2.09-1.88 (m, 4H), 1.81-1.54 (m, 10H), 1.44-1.31 (m, 3H).

The product (2 g) was purified by flash silica gel chromatography (20 g SepaFlash® Silica Flash Column, Eluent of 10% MeOH/EtOAc to 10% MeOH/DCM gradient @100 mL/min). The product was triturated with MTBE/EtOH=2/1 (30 mL) at 15° C. for 14 h. The mixture was filtered and the filter cake was washed with MTBE/EtOH=2:1 (10 mL). The filter cake was collected and then diluted with 1 N HCl (30 mL). The mixture was slowly added to saturated aq. NaHCO₃ (200 mL) and a large quantity of white precipitate was formed. The suspension was filtered and the filter cake was washed with water (50 mL). The filter cake was collected and the solvent was lyophilized removed under reduced pressure. The product was triturated with water (50 mL) at 15° C. for 0.5 h. The suspension was filtered and the filter cake was washed with water (20 mL). The filter cake was collected and the solvent was lyophilized removed under reduced pressure to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-3-fluoro-2-m ethoxyphenyl)piperazin-1-yl)ethyl)piperidin-1-yl)-3-methoxybenzamide (328 mg, 349.34 mol, 9.77% yield, 95% purity) as a yellow solid. MS(M+H)⁺=892.6.

¹H NMR (400 MHz, DMSO-d₆) δ=10.81 (s, 1H), 9.27 (s, 1H), 8.30-8.23 (m, 2H), 7.96 (s, 1H), 7.45-7.39 (m, 2H), 6.60-6.55 (m, 1H), 6.52-6.44 (m, 1H), 5.27 (d, J=6.6 Hz, 1H), 4.82-4.71 (m, 1H), 4.35-4.25 (m, 1H), 4.04 (t, J=13.8 Hz, 2H), 3.93 (s, 3H), 3.82 (s, 3H), 3.35-3.15 (m, 5H), 3.04-2.97 (m, 2H), 2.94-2.82 (m, 4H), 2.81-2.69 (m, 3H), 2.61-2.53 (m, 3H), 2.39-2.30 (m, 2H), 2.12-2.01 (m, 1H), 2.01-1.88 (m, 3H), 1.79-1.66 (m, 4H), 1.65-1.52 (m, 4H), 1.48-1.37 (m, 2H), 1.35-1.24 (m, 3H).

### Example 56. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-5-fluoro-2-methoxyphenyl)piperazin-1-yl)ethyl)piperidin-1-yl)-3-methoxybenzamide (Compound 56)

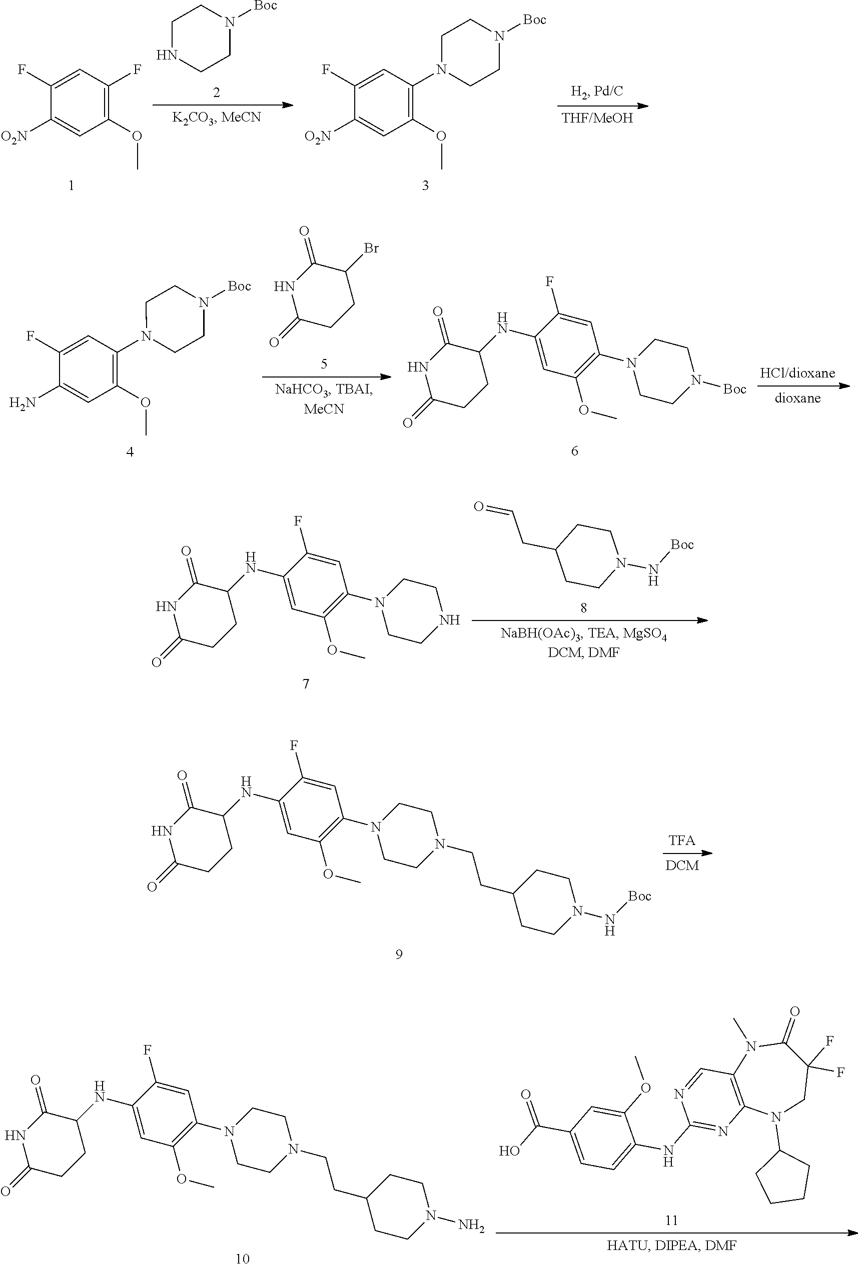

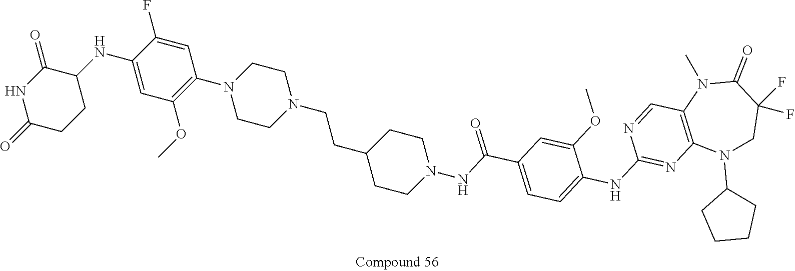 [SMILES: COc1cc(C(=O)NN2CCC(CCN3CCN(c4cc(F)c(NC5CCC(=O)NC5=O)cc4OC)CC3)CC2)ccc1Nc1ncc2c(n1)N(C1CCCC1)CC(F)(F)C(=O)N2C]

### Step 1. Synthesis of tert-butyl 4-(5-fluoro-2-methoxy-4-nitrophenyl)piperazine-1-carboxylate (3)

It was synthesized by similar method described in step 2 of Example 7 to obtain tert-butyl 4-(5-fluoro-2-methoxy-4-nitrophenyl)piperazine-1-carboxylate (7.95 g, 22.37 mmol, 42.74% yield) as a yellow solid. MS(M+H)⁺=356.3.

¹H NMR (400 MHz, CD₃OD) δ=7.61 (d, J=7.2 Hz, 1H), 6.83 (d, J=13.6 Hz, 1H), 3.92 (s, 3H), 3.66-3.50 (m, 4H), 3.26-3.15 (m, 4H), 1.48 (s, 9H).

### Step 2. Synthesis of tert-butyl 4-(4-amino-5-fluoro-2-methoxyphenyl)piperazine-1-carboxylate (4)

To a mixture of Pd/C (2 g, 10% purity) in THF (60 mL) and MeOH (60 mL) was added tert-butyl 4-(5-fluoro-2-methoxy-4-nitrophenyl)piperazine-1-carboxylate (7.95 g, 22.37 mmol) under N₂ atmosphere. The suspension was degassed under vacuum and purged with H₂ several times. The mixture was stirred under H₂ (50 psi) at 25° C. for 16 hours. LCMS showed the starting material was consumed completely and 76% of desired mass. The mixture was filtered, the filter cake was washed with MeOH (200 mL) and THF (200 mL). The filtrate was concentrated in vacuum. The residue was triturated with a mixture (EtOAc:Petroleum ether=1:15, 80 mL) for 10 minutes, the suspension was filtered and the filter cake was washed with Petroleum ether (30 mL), the filter cake was collected and dried to afford tert-butyl 4-(4-amino-5-fluoro-2-methoxyphenyl)piperazine-1-carboxylate (6.56 g, 20.16 mmol, 90.12% yield) as a purple solid. MS(M+H)⁺=326.4.

¹H NMR (400 MHz, DMSO-d₆) δ=6.66 (d, J=12.9 Hz, 1H), 6.46 (d, J=8.6 Hz, 1H), 4.79 (s, 2H), 3.72 (s, 3H), 3.52-3.40 (m, 4H), 2.84-2.73 (m, 4H), 1.45 (s, 9H).

### Step 3. Synthesis of tert-butyl 4-(4-((2,6-dioxopiperidin-3-yl)amino)-5-fluoro-2-methoxyphenyl)piperazine-1-carboxylate (6)

It was synthesized by similar method described in step 3 of Example 51 to obtain tert-butyl 4-(4-((2,6-dioxopiperidin-3-yl)amino)-5-fluoro-2-methoxyphenyl)piperazine-1-carboxylate (2.9 g, 6.64 mmol, 32.95% yield) as a gray solid. MS(M+H)⁺=437.3.

### Step 4. Synthesis of 3-((2-fluoro-5-methoxy-4-(piperazin-1-yl)phenyl)amino)piperidine-2,6-dione (7)

It was synthesized by similar method described in step 5 of Example 1 to obtain 3-((2-fluoro-5-methoxy-4-(piperazin-1-yl)phenyl)amino)piperidine-2,6-dione (3.65 g, HCl salt) as a purple solid, which was used directly. MS(M+H)⁺=337.2.

### Step 5. Synthesis of tert-butyl (4-(2-(4-(4-((2,6-dioxopiperidin-3-yl)amino)-5-fluoro-2-methoxyphenyl)piperazin-1-yl)ethyl)piperidin-1-yl)carbamate (9)

A mixture of 3-((2-fluoro-5-methoxy-4-(piperazin-1-yl)phenyl)amino)piperidine-2,6-dione (3.55 g, 9.52 mmol, HCl salt), TEA (5.45 g, 53.88 mmol, 7.5 mL), tert-butyl (4-(2-oxoethyl)piperidin-1-yl)carbamate (4 g, 16.51 mmol) and MgSO₄ (4 g, 33.23 mmol) in DCM (120 mL) and DMF (120 mL) was stirred at 20° C. for 16 hours, then NaBH(OAc)₃ (6.5 g, 30.67 mmol) was added in portions, the resulting mixture was stirred at 20° C. for 4 hrs. LCMS showed 37% of 3-((2-fluoro-5-methoxy-4-(piperazin-1-yl)phenyl)amino)piperidine-2,6-dione remained and 54% of desired mass. The mixture was stirred at 20° C. for further 12 hours, LCMS showed 3-((2-fluoro-5-methoxy-4-(piperazin-1-yl)phenyl)amino)piperidine-2,6-dione was consumed completely and 92% of desired mass. The mixture was quenched by addition H₂O (400 mL), treated with NaHCO₃ solution to adjust pH>7, and extracted with a mixture (DCM:MeOH=10:1, 200 mL×3). The combined organic layers were washed with brine (300 mL×5), dried over MgSO₄, filtered and concentrated in vacuum. The residue was purified by flash silica gel chromatography (20 g SepaFlash® Silica Flash Column, Eluent of 100% EtOAc to 6% MeOH/DCM gradient @ 150 mL/min) and re-purified by flash silica gel chromatography (20 g SepaFlash® Silica Flash Column, Eluent of 100% EtOAc to 6% MeOH/DCM gradient @ 150 mL/min) to afford tert-butyl (4-(2-(4-(4-((2,6-dioxopiperidin-3-yl)amino)-5-fluoro-2-methoxyphenyl)piperazin-1-yl)ethyl)piperidin-1-yl)carbamate (1.62 g, 2.88 mmol, 30.24% yield) as a blue solid. MS(M+H)⁺=563.5.

### Step 6. Synthesis of 3-((4-(4-(2-(1-aminopiperidin-4-yl)ethyl)piperazin-1-yl)-2-fluoro-5-methoxyphenyl)amino)piperidine-2,6-dione (10)

It was synthesized by similar method described in step 6 of Example 3 to obtain 3-((4-(4-(2-(1-aminopiperidin-4-yl)ethyl)piperazin-1-yl)-2-fluoro-5-methoxyphenyl)amino)piperidine-2,6-dione (2.32 g, 3TFA salt) as a green gum which was used directly. MS(M+H)⁺=463.3.

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-5-fluoro-2-methoxyphenyl)piperazin-1-yl)ethyl)piperidin-1-yl)-3-methoxybenzamide (Compound 56)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-5-fluoro-2-m ethoxyphenyl)piperazin-1-yl)ethyl)piperidin-1-yl)-3-methoxybenzamide (710.60 mg, 783.83 μmol, 62.71% yield, 98.39% purity) as an off-white solid. MS(M+H)⁺=892.5.

¹H NMR (400 MHz, DMSO-d₆) δ=10.78 (s, 1H), 9.31-9.14 (m, 1H), 8.33-8.18 (m, 2H), 7.96 (s, 1H), 7.50-7.30 (m, 2H), 6.67 (d, J=13.4 Hz, 1H), 6.48 (d, J=8.6 Hz, 1H), 5.26-5.09 (m, 1H), 4.82-4.69 (m, 1H), 4.45-4.33 (m, 1H), 4.13-3.99 (m, 2H), 3.93 (s, 3H), 3.72 (s, 3H), 3.32 (s, 3H), 3.00 (d, J=9.9 Hz, 2H), 2.90-2.78 (m, 4H), 2.78-2.67 (m, 3H), 2.60-2.54 (m, 1H), 2.39-2.28 (m, 3H), 2.14-1.95 (m, 3H), 1.95-1.83 (m, 2H), 1.76-1.67 (m, 4H), 1.67-1.49 (m, 5H), 1.45-1.37 (m, 2H), 1.37-1.04 (m, 4H).

### Example 57. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,5-difluorophenyl)piperazin-1-yl)ethyl)piperidin-1-yl)-3-methoxybenzamide (Compound 57)

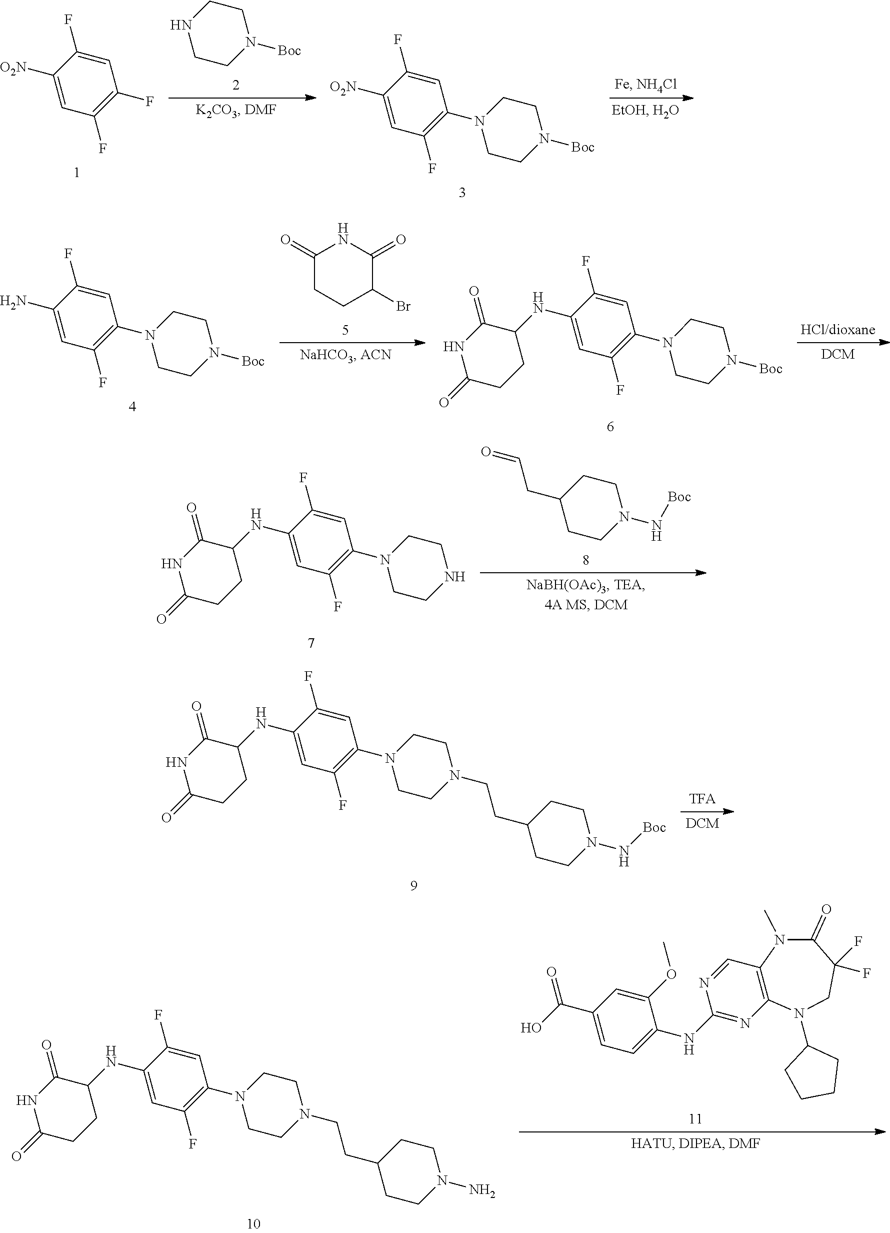

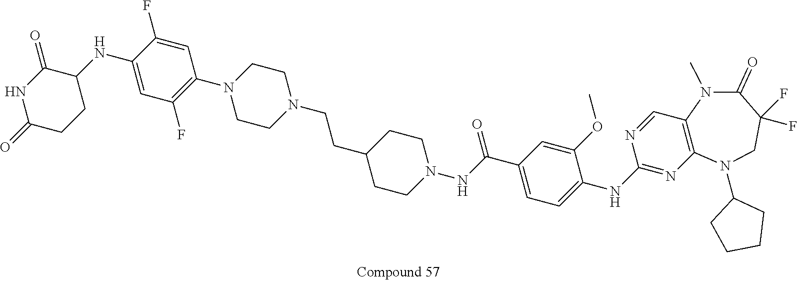 [SMILES: COc1cc(C(=O)NN2CCC(CCN3CCN(c4cc(F)c(NC5CCC(=O)NC5=O)cc4F)CC3)CC2)ccc1Nc1ncc2c(n1)N(C1CCCC1)CC(F)(F)C(=O)N2C]

### Step 1. Synthesis of tert-butyl 4-(2,5-difluoro-4-nitrophenyl)piperazine-1-carboxylate (3)

It was synthesized by similar method described in step 4 of Example 30 to obtain tert-butyl 4-(2,5-difluoro-4-nitrophenyl)piperazine-1-carboxylate (1.3 g, 3.75 mmol, 66.38% yield, 99% purity) as a yellow solid. MS(M+H-Boc)⁺=244.2

¹H NMR (400 MHz, CDCl₃) δ=7.83 (dd, J=7.1, 12.8 Hz, 1H), 6.64 (dd, J=7.2, 13.0 Hz, 1H), 3.71-3.51 (m, 4H), 3.35-3.16 (m, 4H), 1.49 (s, 9H).

### Step 2. Synthesis of tert-butyl 4-(4-amino-2,5-difluorophenyl)piperazine-1-carboxylate (4)

It was synthesized by similar method described in step 4 of Example 22 to obtain tert-butyl 4-(4-amino-2,5-difluorophenyl)piperazine-1-carboxylate (1.1 g, 3.16 mmol, 83.44% yield, 90% purity) as yellow oil. MS(M+H-Boc)⁺=214.0

### Step 3. Synthesis of tert-butyl 4-(4-((2,6-dioxopiperidin-3-yl)amino)-2,5-difluorophenyl)piperazine-1-carboxylate (6)

It was synthesized by similar method described in step 5 of Example 22 to obtain tert-butyl 4-(4-((2,6-dioxopiperidin-3-yl)amino)-2,5-difluorophenyl)piperazine-1-carboxylate (1.2 g, 2.43 mmol, 69.26% yield, 86% purity) as a brown solid. MS(M+H)⁺=425.3

### Step 4. Synthesis of 3-((2,5-difluoro-4-(piperazin-1-yl)phenyl)amino)piperidine-2,6-dione (7)

It was synthesized by similar method described in step 2 of Example 48 to obtain 3-((2,5-difluoro-4-(piperazin-1-yl)phenyl)amino)piperidine-2,6-dione (0.8 g, crude, HCl) as a blue solid. MS(M+H)⁺=325.2

### Step 5. Synthesis of tert-butyl (4-(2-(4-(4-((2,6-dioxopiperidin-3-yl)amino)-2,5-difluorophenyl)piperazin-1-yl)eth yl)piperidin-1-yl)carbamate (9)

It was synthesized by similar method described in step 6 of Example 1 to obtain tert-butyl (4-(2-(4-(4-((2,6-dioxopiperidin-3-yl)amino)-2,5-difluorophenyl)piperazin-1-yl)ethyl)piperidin-1-yl)carbamate (0.23 g, 396.81 mol, 16.09% yield, 95% purity) as a gray solid. MS(M+H)⁺=551.3

### Step 6. Synthesis of 3-((4-(4-(2-(1-aminopiperidin-4-yl)ethyl)piperazin-1-yl)-2,5-difluorophenyl)amino)piperidine-2,6-dione (10)

It was synthesized by similar method described in step 6 of Example 3 to obtain 3-((4-(4-(2-(1-aminopiperidin-4-yl)ethyl)piperazin-1-yl)-2,5-difluorophenyl)amino)piperidine-2,6-dione (0.17 g, 301.13 mol, 97.54% yield, TFA) as yellow oil. MS(M+H)⁺=451.2

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,5-difluorophenyl)piperazin-1-yl)ethyl)piperidin-1-yl)-3-methoxybenzamide (Compound 57)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,5-difluoro phenyl)piperazin-1-yl)ethyl)piperidin-1-yl)-3-methoxybenzamide (72.6 mg, 72.31 mol, 24.01% yield, 99% purity, TFA) as a white solid. MS(M+H)⁺=880.4.

¹H NMR (400 MHz, DMSO-d₆) δ=10.81 (s, 1H), 9.82-9.61 (m, 1H), 9.58-9.39 (m, 1H), 8.33-8.11 (m, 3H), 7.53-7.38 (m, 2H), 7.08-6.89 (m, 1H), 6.79 (br dd, J=8.2, 14.3 Hz, 1H), 4.82-4.76 (m, 1H), 4.39 (br t, J=8.2 Hz, 1H), 4.09 (br t, J=13.9 Hz, 2H), 3.94 (s, 3H), 3.62-3.54 (m 2H), 3.33 (s, 5H), 3.26-3.09 (m, 6H), 3.02-2.81 (m, 4H), 2.79-2.69 (m, 1H), 2.07-1.99 (m, 2H), 1.99-1.90 (m, 2H), 1.84-1.54 (m, 11H), 1.42-1.36 (m, 3H).

### Example 58. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,5-difluorophenyl)piperazin-1-yl)ethyl)piperidin-1-yl)-3-methoxybenzamide (Compound 58)

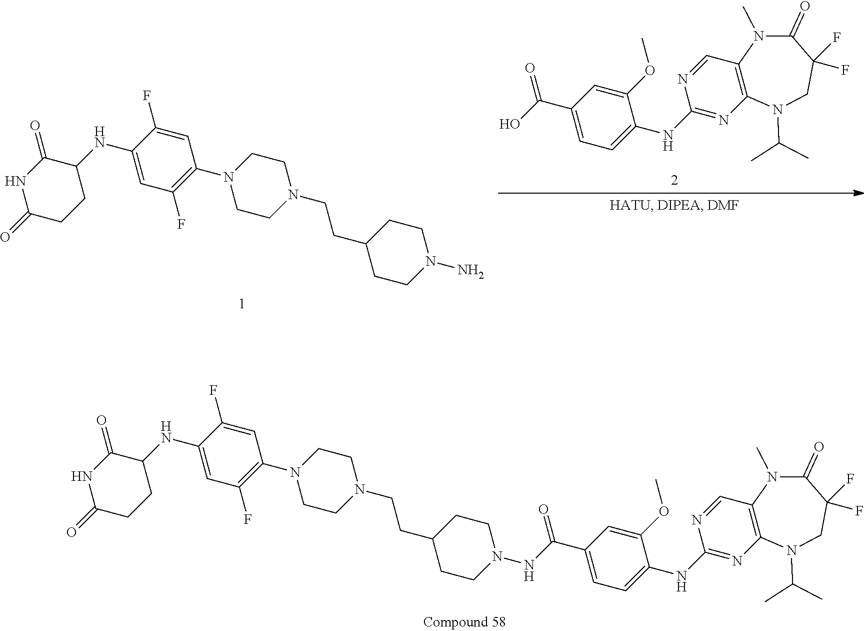 [SMILES: NN1CCC(CCN2CCN(c3cc(F)c(NC4CCC(=O)NC4=O)cc3F)CC2)CC1 ; COc1cc(C(=O)O)ccc1Nc1ncc2c(n1)N(C(C)C)CC(F)(F)C(=O)N2C ; COc1cc(C(=O)NN2CCC(CCN3CCN(c4cc(F)c(NC5CCC(=O)NC5=O)cc4F)CC3)CC2)ccc1Nc1ncc2c(n1)N(C(C)C)CC(F)(F)C(=O)N2C]

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,5-difluorophenyl)piperazin-1-yl)ethyl)piperidin-1-yl)-3-methoxybenzamide (110.4 mg, 97.65 μmol, 40.84% yield, 95.7% purity, 2TFA) as a white solid. MS(M+H)⁺=854.4

¹H NMR (400 MHz, DMSO-d₆) δ=10.81 (s, 1H), 9.69-9.32 (m, 2H), 8.27 (br d, J=8.2 Hz, 1H), 8.23 (s, 1H), 8.17-8.10 (m, 1H), 7.50-7.41 (m, 2H), 6.97 (dd, J=8.3, 13.0 Hz, 1H), 6.78 (dd, J=8.4, 14.6 Hz, 1H), 5.75-5.51 (m, 1H), 4.92-4.85 (m, 1H), 4.40-4.36 (m, 1H), 4.10-4.04 (m, 2H), 3.93 (s, 3H), 3.61-3.52 (m, 2H), 3.37-3.29 (m, 5H), 3.24-3.07 (m, 6H), 3.00-2.90 (m, 2H), 2.87-2.74 (m, 2H), 2.73-2.54 (m, 1H), 2.07-1.99 (m, 2H), 1.80-1.70 (m, 2H), 1.64 (br s, 2H), 1.45-1.30 (m, 3H), 1.24 (d, J=6.7 Hz, 6H).

### Example 59. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,5-difluorophenyl)piperazin-1-yl)ethyl)piperidin-1-yl)-2-fluoro-5-methoxybenzamide (Compound 59)

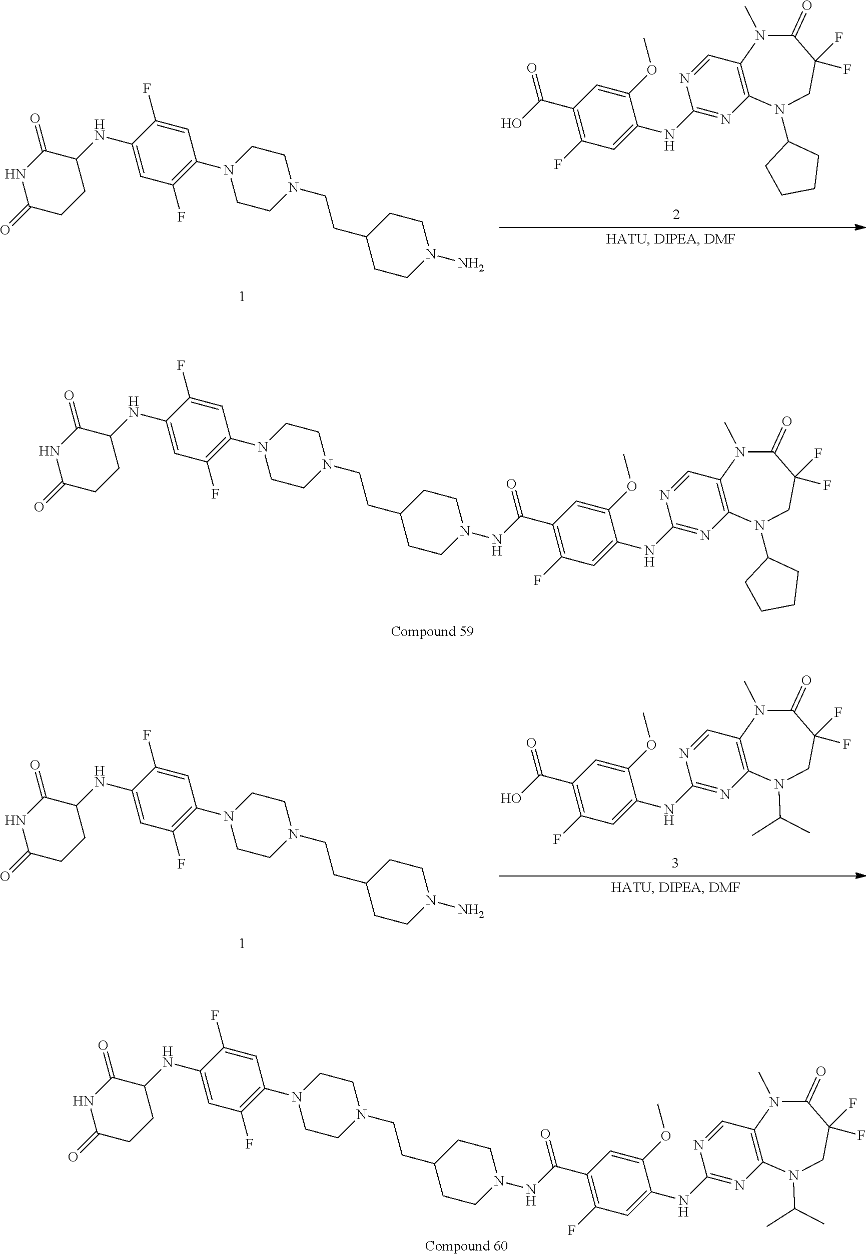 [SMILES: NN1CCC(CCN2CCN(c3cc(F)c(NC4CCC(=O)NC4=O)cc3F)CC2)CC1 ; COc1cc(C(=O)O)c(F)cc1Nc1ncc2c(n1)N(C1CCCC1)CC(F)(F)C(=O)N2C ; COc1cc(C(=O)NN2CCC(CCN3CCN(c4cc(F)c(NC5CCC(=O)NC5=O)cc4F)CC3)CC2)c(F)cc1Nc1ncc2c(n1)N(C1CCCC1)CC(F)(F)C(=O)N2C ; NN1CCC(CCN2CCN(c3cc(F)c(NC4CCC(=O)NC4=O)cc3F)CC2)CC1 ; COc1cc(C(=O)O)c(F)cc1Nc1ncc2c(n1)N(C(C)C)CC(F)(F)C(=O)N2C ; COc1cc(C(=O)NN2CCC(CCN3CCN(c4cc(F)c(NC5CCC(=O)NC5=O)cc4F)CC3)CC2)c(F)cc1Nc1ncc2c(n1)N(C(C)C)CC(F)(F)C(=O)N2C]

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,5-difluoro phenyl)piperazin-1-yl)ethyl)piperidin-1-yl)-2-fluoro-5-methoxybenzamide (1.52 g, 1.66 mmol, 35.97% yield, 98% purity) as a yellow solid. MS(M+H)⁺=898.4.

¹H NMR (400 MHz, DMSO-d₆) δ=10.80 (s, 1H), 9.00 (s, 1H), 8.30 (s, 1H), 8.28-8.21 (m, 1H), 8.04 (s, 1H), 7.14 (d, J=6.4 Hz, 1H), 6.84 (br dd, J=8.1, 13.0 Hz, 1H), 6.73 (br dd, J=8.2, 14.6 Hz, 1H), 5.49 (br d, J=8.4 Hz, 1H), 4.88-4.77 (m, 1H), 4.38-4.31 (m, 1H), 4.08 (br t, J=13.9 Hz, 2H), 3.91 (s, 3H), 3.33 (s, 3H), 3.09-2.98 (m 2H), 2.95-2.84 (m, 5H), 2.77-2.62 (m, 5H), 2.39-2.30 (m, 2H), 2.09-1.92 (m, 5H), 1.77-1.59 (m, 9H), 1.44-1.27 (m, 5H).

### Example 60. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,5-difluorophenyl)piperazin-1-yl)ethyl)piperidin-1-yl)-2-fluoro-5-methoxybenzamide (Compound 60)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,5-difluorophenyl)piperazin-1-yl)ethyl)piperidin-1-yl)-2-fluoro-5-methoxybenzamide (1.60 g, 1.79 mmol, 65.04% yield, 97.4% purity) as a yellow solid. MS(M+H)⁺=872.4.

¹H NMR (400 MHz, DMSO-d₆) δ=10.80 (s, 1H), 8.98 (d, J=1.8 Hz, 1H), 8.33-8.16 (m, 2H), 7.97 (s, 1H), 7.14 (d, J=6.5 Hz, 1H), 6.84 (dd, J=8.1, 13.1 Hz, 1H), 6.78-6.68 (m, 1H), 5.49 (br d, J=8.1 Hz, 1H), 4.90 (td, J=6.6, 13.4 Hz, 1H), 4.42-4.28 (m, 1H), 4.06 (br t, J=13.4 Hz, 2H), 3.91 (s, 3H), 3.39-3.31 (m, 5H), 3.10-3.01 (m, 2H), 2.91-2.81 (m, 4H), 2.80-2.56 (m, 4H), 2.48-2.41 (m, 2H), 2.39-2.28 (m, 2H), 2.12-1.96 (m, 2H), 1.78-1.70 (m, 2H), 1.47-1.38 (m, 2H), 1.36-1.21 (m, 9H).

### Example 61. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-(2-(1-(4-((2,6-dioxopiperidin-3-yl)amino)-2,5-difluorophenyl)piperidin-4-yl)ethyl)-7-azaspiro[3.5]nonan-2-yl)-3-methoxybenzamide (Compound 61)

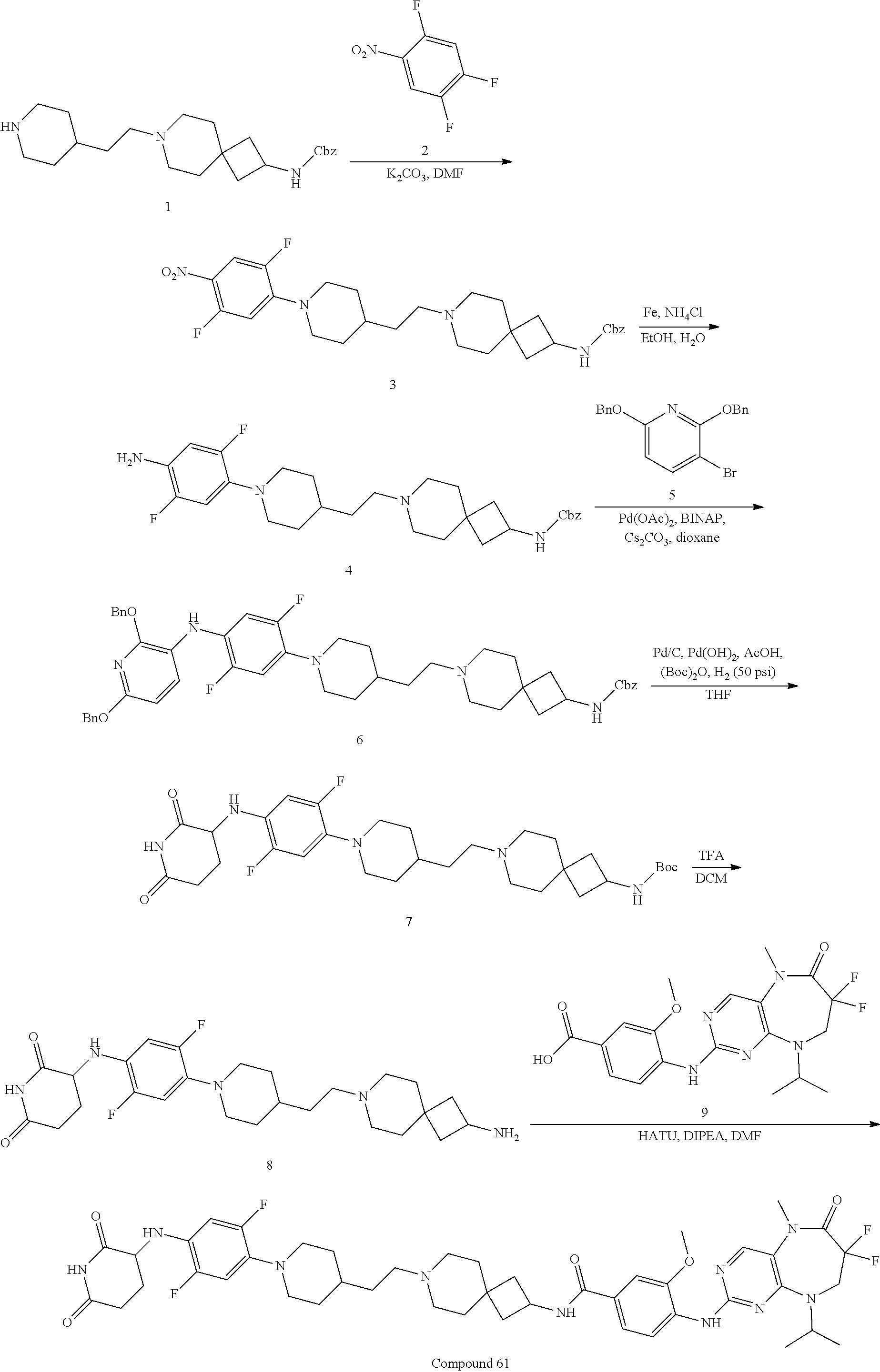

### Step 1. Synthesis of benzyl (7-(2-(1-(2,5-difluoro-4-nitrophenyl)piperidin-4-yl)ethyl)-7-azaspiro[3.5]nonan-2-yl)carbamate (3)

It was synthesized by similar method described in step 4 of Example 30 to obtain benzyl (7-(2-(1-(2,5-difluoro-4-nitrophenyl)piperidin-4-yl)ethyl)-7-azaspiro[3.5]nonan-2-yl)carbamate (3.9 g, 7.19 mmol, 97.84% yield) as a yellow oil. MS(M+H)⁺=543.3.

### Step 2. Synthesis of benzyl (7-(2-(1-(4-amino-2,5-difluorophenyl)piperidin-4-yl)ethyl)-7-azaspiro[3.5]nonan-2-yl)carbamate (4)

It was synthesized by similar method described in step 4 of Example 22 to obtain benzyl (7-(2-(1-(4-amino-2,5-difluorophenyl)piperidin-4-yl)ethyl)-7-azaspiro[3.5]nonan-2-yl) carbamate (3.6 g, 7.02 mmol, 97.71% yield) as a brown oil. MS(M+H)⁺=513.3.

### Step 3. Synthesis of benzyl (7-(2-(1-(4-((2,6-bis(benzyloxy)pyridine-3-yl)amino)-2,5-difluorophenyl)piperidin-4-yl)ethyl)-7-azaspiro[3.5]nonan-2-yl)carbamate (6)

It was synthesized by similar method described in step 3 of Example 1 to obtain benzyl (7-(2-(1-(4-((2,6-bis(benzyloxy)pyridin-3-yl)amino)-2,5-difluorophenyl)piperidin-4-yl) ethyl)-7-azaspiro[3.5]nonan-2-yl)carbamate (1 g, 1.25 mmol, 35.51% yield) as a brown oil. MS(M+H)⁺=802.2.

### Step 4. Synthesis of tert-butyl (7-(2-(1-(4-((2,6-dioxopiperidin-3-yl)amino)-2,5-difluorophenyl)piperidin-4-yl)eth yl)-7-azaspiro[3.5]nonan-2-yl)carbamate (7)

It was synthesized by similar method described in step 4 of Example 51, addition Pd(OH)₂ (0.5 g, 712.07 μmol, 20% purity) to obtain tert-butyl (7-(2-(1-(4-((2,6-dioxopiperidin-3-yl)amino)-2,5-difluorophenyl)piperidin-4-yl)ethyl)-7-azaspiro[3.5]nonan-2-yl)carbamate (400 mg, 678.29 mol, 77.71% yield) as a blue solid. MS(M+H)⁺=590.4.

### Step 5. Synthesis of 3-((4-(4-(2-(2-amino-7-azaspiro[3.5]nonan-7-yl)ethyl)piperidin-1-yl)-2,5-difluorophenyl)amino)piperidine-2,6-dione (8)

It was synthesized by similar method described in step 6 of Example 3 to obtain 3-((4-(4-(2-(2-amino-7-azaspiro[3.5]nonan-7-yl)ethyl)piperidin-1-yl)-2,5-difluorophenyl)amino)piperidine-2,6-dione (500 mg, 828.33 mol, 97.70% yield, TFA salt) as a brown oil. MS(M+H)⁺=490.3.

### Step 6. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-(2-(1-(4-((2,6-dioxopiperidin-3-yl)amino)-2,5-difluorophenyl)piperidin-4-yl)ethyl)-7-azaspiro[3.5]nonan-2-yl)-3-methoxybenzamide (Compound 61)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(7-(2-(1-(4-((2,6-dioxopiperidin-3-yl)amino)-2,5-difluorophenyl)piperidin-4-yl)ethyl)-7-azaspiro[3.5]nonan-2-yl)-3-methoxybenzamide (299.4 mg, 325.22 μmol, 39.26% yield, 97% purity) as a white solid. MS(M+H)⁺=893.4.

¹H NMR (400 MHz, DMSO-d₆) δ=10.79 (s, 1H), 8.45-8.38 (m, 1H), 8.30 (d, J=8.4 Hz, 1H), 8.22 (s, 1H), 7.88 (s, 1H), 7.54-7.46 (m, 2H), 6.87-6.78 (m, 1H), 6.76-6.66 (m, 1H), 5.49-5.42 (m, 1H), 4.95-4.82 (m, 1H), 4.46-4.29 (m, 2H), 4.09-3.99 (m, 2H), 3.94 (s, 3H), 3.33-3.32 (m, 3H), 3.18-3.05 (m, 2H), 2.80-2.65 (m, 1H), 2.60-2.51 (m, 3H), 2.34-2.11 (m, 8H), 2.08-1.97 (m, 2H), 1.84-1.77 (m, 2H), 1.75-1.68 (m, 2H), 1.61-1.51 (m, 4H), 1.42-1.35 (m, 2H), 1.34-1.27 (m, 2H), 1.24 (d, J=6.7 Hz, 7H).

### Example 62. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2,5-difluorophenyl)piperidin-4-yl)ethyl)piperidin-4-yl)-3-methoxybenzamide (Compound 62)

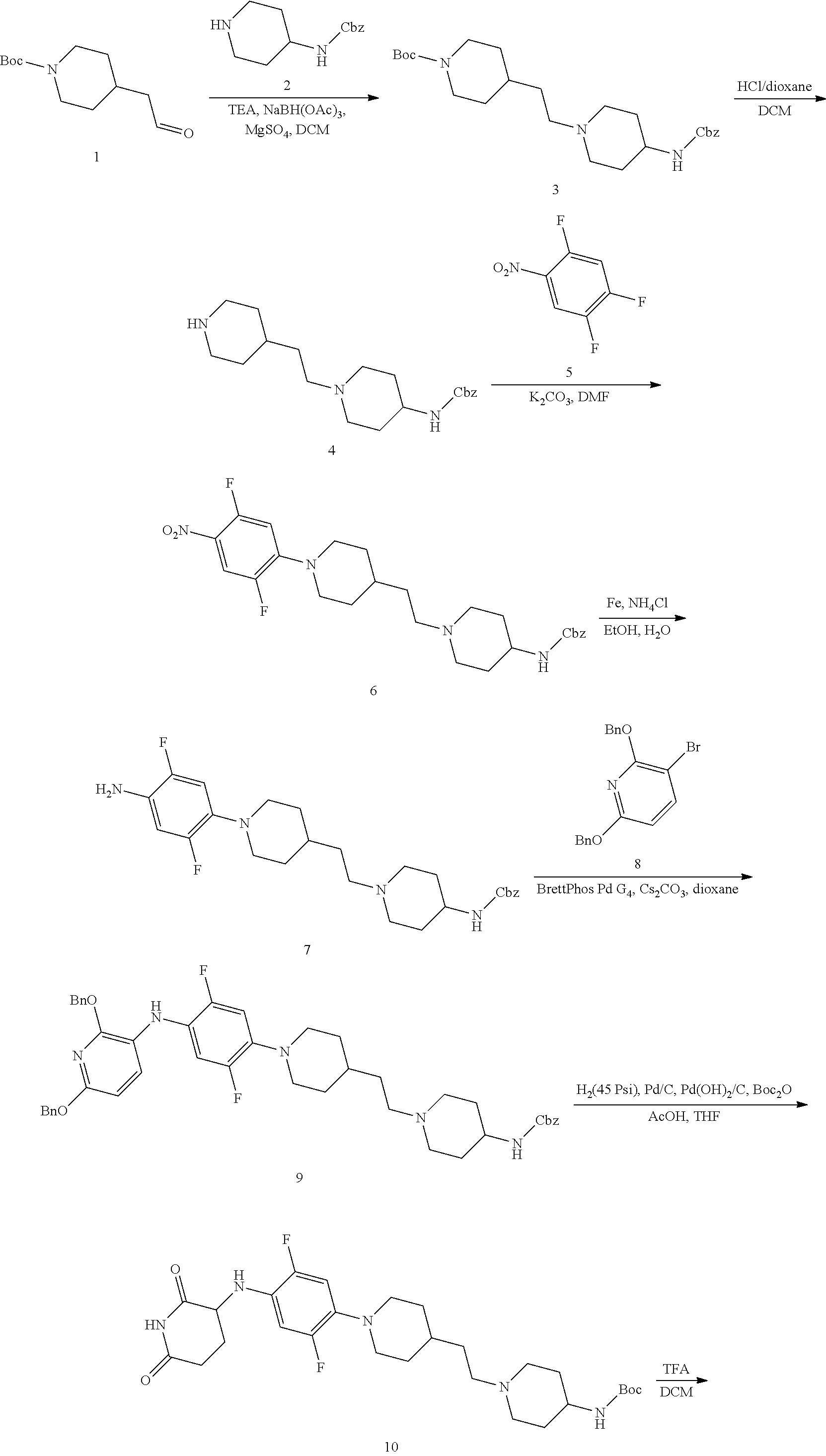

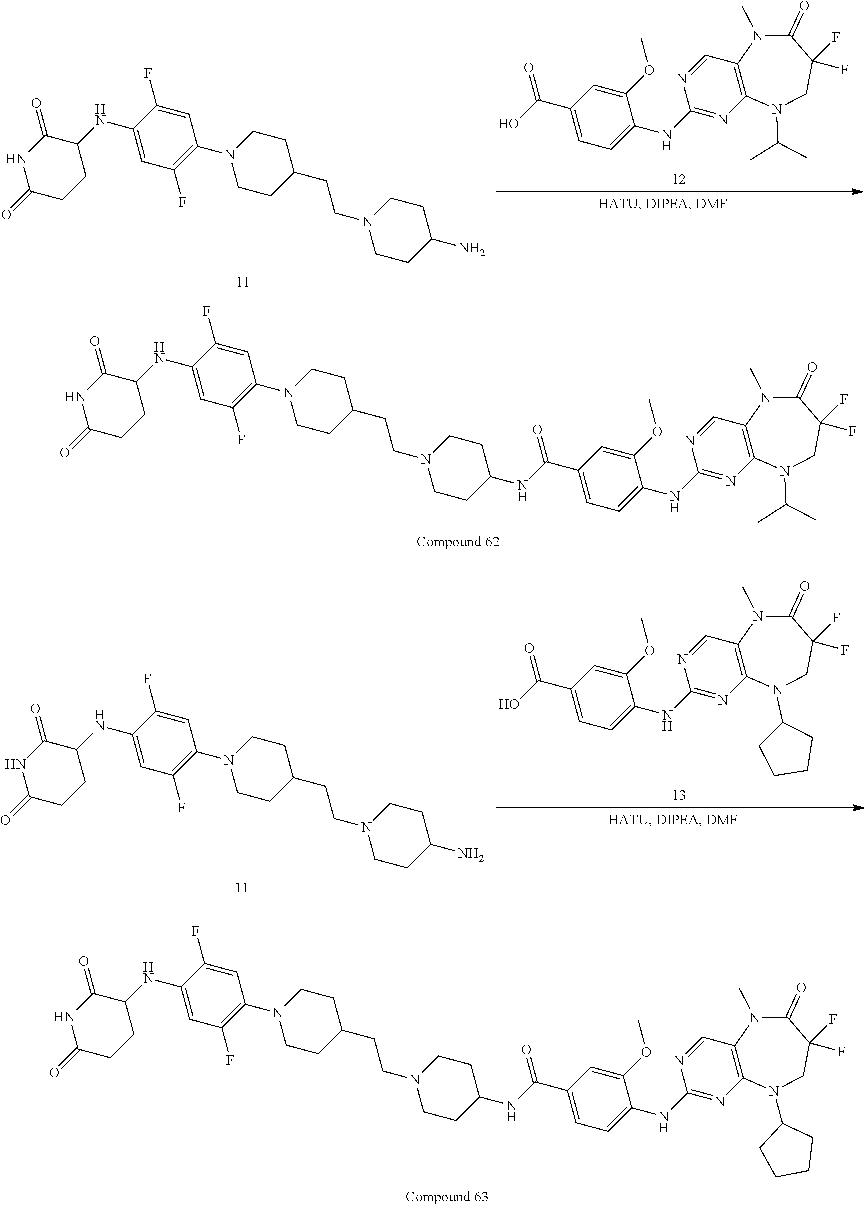 [SMILES: NC1CCN(CCC2CCN(c3cc(F)c(NC4CCC(=O)NC4=O)cc3F)CC2)CC1 ; COc1cc(C(=O)O)ccc1Nc1ncc2c(n1)N(C(C)C)CC(F)(F)C(=O)N2C ; COc1cc(C(=O)NC2CCN(CCC3CCN(c4cc(F)c(NC5CCC(=O)NC5=O)cc4F)CC3)CC2)ccc1Nc1ncc2c(n1)N(C(C)C)CC(F)(F)C(=O)N2C ; NC1CCN(CCC2CCN(c3cc(F)c(NC4CCC(=O)NC4=O)cc3F)CC2)CC1 ; COc1cc(C(=O)O)ccc1Nc1ncc2c(n1)N(C1CCCC1)CC(F)(F)C(=O)N2C ; COc1cc(C(=O)NC2CCN(CCC3CCN(c4cc(F)c(NC5CCC(=O)NC5=O)cc4F)CC3)CC2)ccc1Nc1ncc2c(n1)N(C1CCCC1)CC(F)(F)C(=O)N2C]

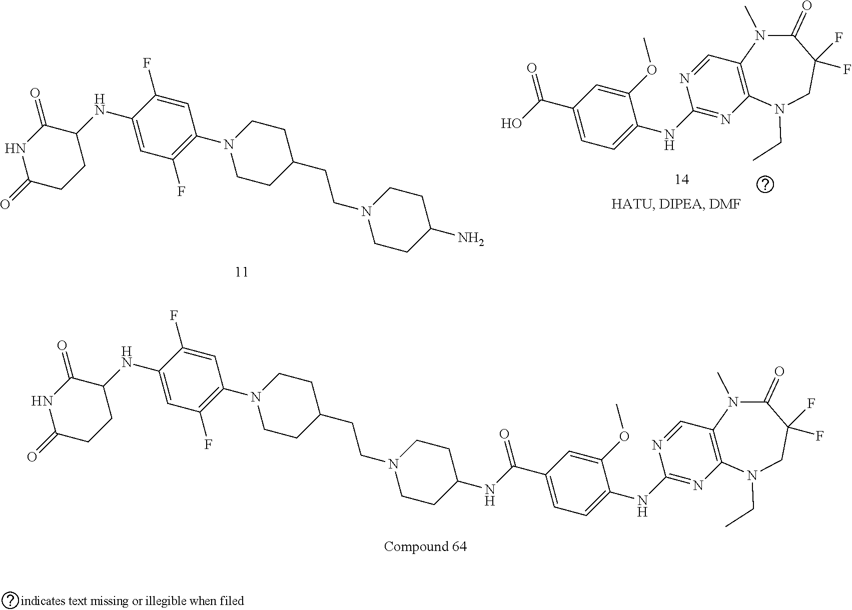 [SMILES: NC1CCN(CCC2CCN(c3cc(F)c(NC4CCC(=O)NC4=O)cc3F)CC2)CC1 ; CCN1CC(F)(F)C(=O)N(C)c2cnc(Nc3ccc(C(=O)O)cc3OC)nc21 ; CCN1CC(F)(F)C(=O)N(C)c2cnc(Nc3ccc(C(=O)NC4CCN(CCC5CCN(c6cc(F)c(NC7CCC(=O)NC7=O)cc6F)CC5)CC4)cc3OC)nc21]

### Step 1. Synthesis of tert-butyl 4-(2-(4-(((benzyloxy)carbonyl)amino)piperidin-1-yl)ethyl)piperidine-1-carboxylate (3)

It was synthesized similar method described in step 6 of Example 1 to obtain tert-butyl 4-(2-(4-(((benzyloxy)carbonyl)amino)piperidin-1-yl)ethyl)piperidine-1l-carboxylate (16 g, crude) as a white solid. MS(M+H)⁺=446.4

### Step 2. Synthesis of benzyl (1-(2-(piperidin-4-yl)ethyl)piperidin-4-yl)carbamate (4)

It was synthesized similar method described in step 2 of Example 48 to obtain benzyl (1-(2-(piperidin-4-yl)ethyl)piperidin-4-yl)carbamate (10 g, crude, HCl) as a white solid. MS(M+H)⁺=346.3

### Step 3. Synthesis of benzyl (1-(2-(1-(2,5-difluoro-4-nitrophenyl)piperidin-4-yl)ethyl)piperidin-4-yl)carbamate (6)

It was synthesized similar method described in step 4 of Example 30 to obtain benzyl (1-(2-(1-(2,5-difluoro-4-nitrophenyl)piperidin-4-yl)ethyl)piperidin-4-yl)carbamate (5 g, 9.45 mmol, 72.20% yield, 95% purity) as a yellow solid. MS(M+H)⁺=503.3

### Step 4. Synthesis of benzyl (1-(2-(1-(4-amino-2,5-difluorophenyl)piperidin-4-yl)ethyl)piperidin-4-yl)carbamate (7)

It was synthesized similar method described in step 4 of Example 22 to obtain benzyl (1-(2-(1-(4-amino-2,5-difluorophenyl)piperidin-4-yl)ethyl)piperidin-4-yl)carbamate (1.48 g, 3.13 mmol, 62.96% yield, 100% purity) as a yellow solid. MS(M+H)⁺=473.3

### Step 5. Synthesis of benzyl (1-(2-(1-(4-((2,6-bis(benzyloxy)pyridin-3-yl)amino)-2,5-difluorophenyl)piperidin-4-yl)ethyl)piperidin-4-yl)carbamate (9)

It was synthesized similar method described in step 1 of Example 36 to obtain benzyl (1-(2-(1-(4-((2,6-bis(benzyloxy)pyridin-3-yl)amino)-2,5-difluorophenyl)piperidin-4-yl) ethyl)piperidin-4-yl)carbamate (1.26 g, 1.65 mmol, 78.15% yield) as a yellow solid. MS(M+H)⁺=762.4

### Step 6. Synthesis of tert-butyl (1-(2-(1-(4-((2,6-dioxopiperidin-3-yl)amino)-2,5-difluorophenyl)piperidin-4-yl)eth yl)piperidin-4-yl)carbamate (10)

It was synthesized similar method described in step 4 of Example 61 to obtain tert-butyl (1-(2-(1-(4-((2,6-dioxopiperidin-3-yl)amino)-2,5-difluorophenyl)piperidin-4-yl)ethyl)piperidin-4-yl)carbamate (440 mg, 800.51 mol, 57.54% yield, 100% purity) as a blue solid. MS(M+H)⁺=550.4

### Step 7. Synthesis of 3-((4-(4-(2-(4-aminopiperidin-1-yl)ethyl)piperidin-1-yl)-2,5-difluorophenyl)amino) piperidine-2,6-dione (11)

It was synthesized similar method described in step 6 of Example 3 to obtain 3-((4-(4-(2-(4-aminopiperidin-1-yl)ethyl)piperidin-1-yl)-2,5-difluorophenyl)amino)piperidine-2,6-dione (450 mg, crude, TFA) as a blue oil. MS(M+H)⁺=450.4

### Step 8. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2,5-difluorophenyl)piperidin-4-yl)ethyl)piperidin-4-yl)-3-methoxybenzamide (Compound 62)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2,5-difluorophenyl)piperidin-4-yl)ethyl)piperidin-4-yl)-3-methoxybenzamide (237.1 mg, 272.43 μmol, 35.88% yield, 98% purity) as a white solid. MS(M+H)⁺=853.5

¹H NMR (400 MHz, DMSO-d₆) δ=10.79 (s, 1H), 8.30 (d, J=8.2 Hz, 1H), 8.22 (s, 1H), 8.10 (d, J=7.7 Hz, 1H), 7.87 (s, 1H), 7.54-7.45 (m, 2H), 6.83 (dd, J=8.1, 13.1 Hz, 1H), 6.71 (dd, J=8.4, 14.3 Hz, 1H), 5.49-5.42 (m, 1H), 4.94-4.81 (m, 1H), 4.39-4.29 (m, 1H), 4.03 (t, J=13.6 Hz, 2H), 3.93 (s, 3H), 3.83-3.71 (m, 1H), 3.26 (s, 3H), 3.16-3.08 (m, 2H), 2.93-2.85 (m, 2H), 2.78-2.66 (m, 1H), 2.59-2.52 (m, 3H), 2.35-2.28 (m, 2H), 2.07-2.00 (m, 2H), 1.98-1.88 (m, 2H), 1.82-1.69 (m, 4H), 1.63-1.51 (m, 2H), 1.45-1.28 (m, 5H), 1.24 (d, J=6.7 Hz, 6H).

### Example 63. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2,5-difluorophenyl)piperidin-4-yl)ethyl)piperidin-4-yl)-3-methoxybenzamide (Compound 63)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(4-((2,6-dioxopiperidin-3-yl)amino)-2,5-difluoro phenyl)piperidin-4-yl)ethyl)piperidin-4-yl)-3-methoxybenzamide (130.7 mg, 145.73 mol, 17.16% yield, 98% purity) as gray solid. MS(M+H)⁺=879.5

¹H NMR (400 MHz, DMSO-d₆) δ=10.79 (s, 1H), 8.30-8.23 (m, 2H), 8.11 (d, J=7.7 Hz, 1H), 7.96 (s, 1H), 7.53-7.44 (m, 2H), 6.83 (dd, J=8.1, 13.2 Hz, 1H), 6.71 (dd, J=8.3, 14.3 Hz, 1H), 5.48-5.43 (m, 1H), 4.83-4.70 (m, 1H), 4.38-4.28 (m, 1H), 4.04 (t, J=14.1 Hz, 2H), 3.93 (s, 3H), 3.84-3.70 (m, 1H), 3.32 (s, 3H), 3.17-3.08 (m, 2H), 2.93-2.85 (m, 2H), 2.79-2.66 (m, 1H), 2.60-2.53 (m, 3H), 2.36-2.29 (m, 2H), 2.09-1.86 (m, 6H), 1.82-1.67 (m, 6H), 1.65-1.53 (m, 6H), 1.45-1.22 (m, 5H)

### Example 64. Synthesis of N(1-(2-(1-(4-((2,6-dioxopiperidin-3-yl)amino)-2,5-difluorophenyl)piperidin-4-yl)eth yl)piperidin-4-yl)-4-((9-ethyl-7,7-difluoro-5-methyl-6-oxo-6,7,8,9-tetrahydro-5H-pyrimido[4,5-b][1,4]diazepin-2-yl)amino)-3-methoxybenzamide (Compound 64)

It was synthesized by similar method described in step 10 of Example 2 to obtain N(1-(2-(1-(4-((2,6-dioxopiperidin-3-yl)amino)-2,5-difluorophenyl)piperidin-4-yl)ethyl)piperidin-4-yl)-4-((9-ethyl-7,7-difluoro-5-methyl-6-oxo-6,7,8,9-tetrahydro-5H-pyrimido [4,5-b][1,4]diazepin-2-yl)amino)-3-methoxybenzamide (258.3 mg, 304.83 μmol, 35.48% yield, 99% purity) as a gray solid. MS(M+H)⁺=839.4

¹H NMR (400 MHz, DMSO-d₆) δ=10.79 (s, 1H), 8.31 (d, J=8.3 Hz, 1H), 8.23 (s, 1H), 8.10 (d, J=7.7 Hz, 1H), 7.89 (s, 1H), 7.55-7.47 (m, 2H), 6.83 (dd, J=8.1, 13.3 Hz, 1H), 6.72 (dd, J=8.4, 14.4 Hz, 1H), 5.49-5.43 (m, 1H), 4.38-4.30 (m, 1H), 4.17 (t, J=13.4 Hz, 2H), 3.94 (s, 3H), 3.82-3.72 (m, 1H), 3.69-3.60 (m, 2H), 3.33 (s, 3H), 3.17-3.09 (m, 2H), 2.94-2.85 (m, 2H), 2.79-2.68 (m, 1H), 2.60-2.53 (m, 3H), 2.36-2.27 (m, 2H), 2.07-1.89 (m, 4H), 1.83-1.70 (m, 4H), 1.64-1.52 (m, 2H), 1.46-1.25 (m, 5H), 1.22 (t, J=7.0 Hz, 3H).

### Example 65. Synthesis of 3-((4-(4-(2-(1-((5-(4-((9-cyclopentyl-7,7-difluoro-5-methyl-6-oxo-6,7,8,9-tetrahydro-5H-pyrimido[4,5-b][1,4]diazepin-2-yl)amino)-3-methoxyphenyl)-1,3,4-oxadiazol-2-yl)methyl)piperidin-4-yl)ethyl)piperazin-1-yl)-2,5-difluorophenyl)amino)piperidine-2,6-dione (Compound 65)

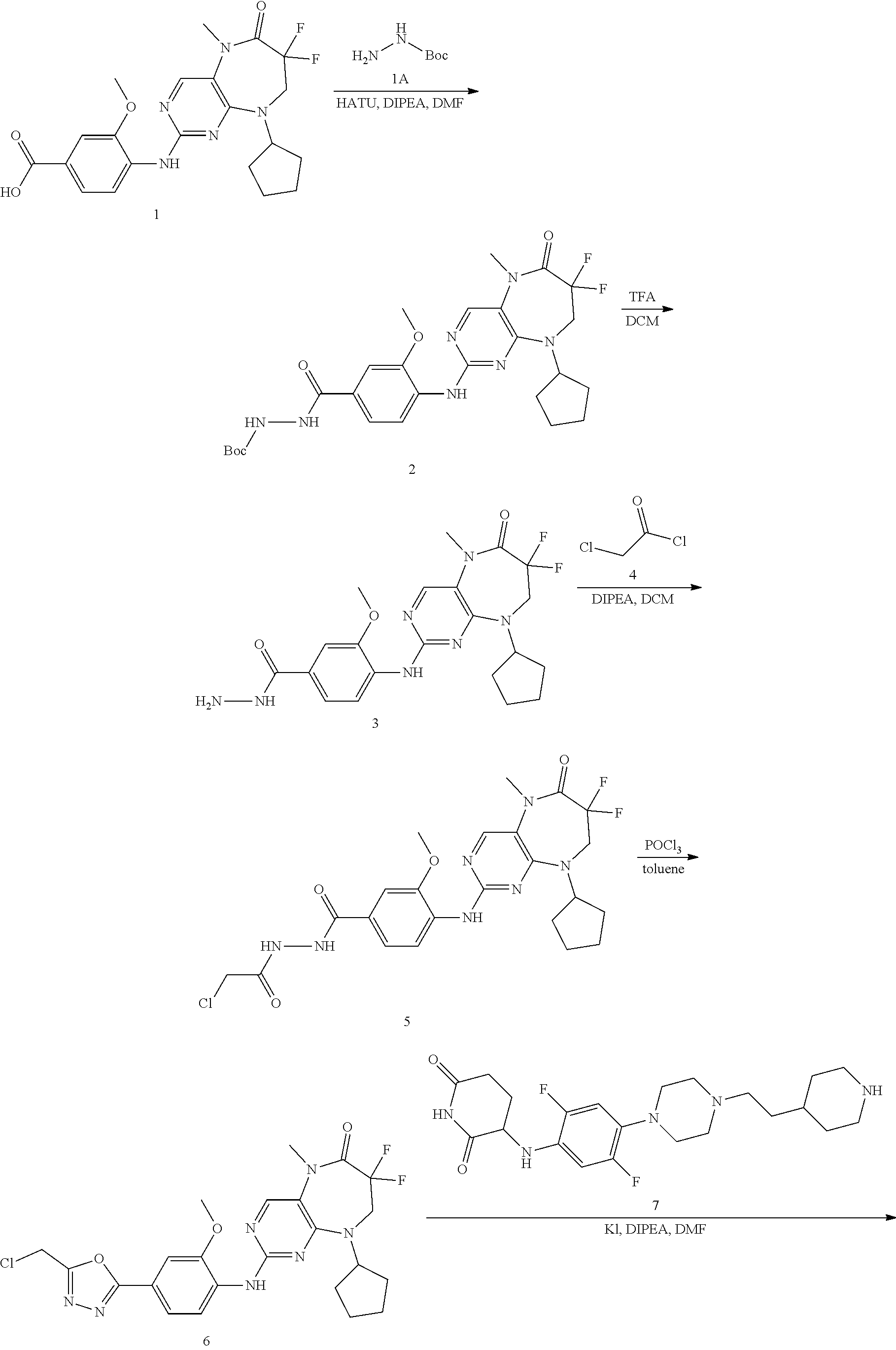

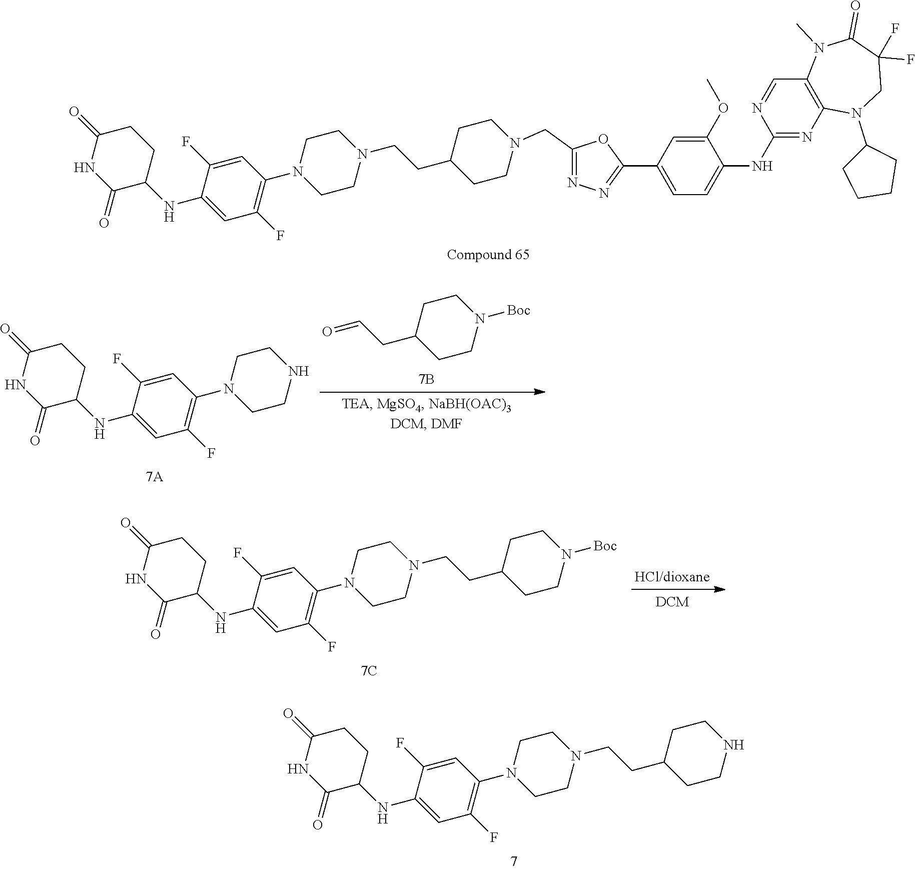 [SMILES: COc1cc(-c2nnc(CN3CCC(CCN4CCN(c5cc(F)c(NC6CCC(=O)NC6=O)cc5F)CC4)CC3)o2)ccc1Nc1ncc2c(n1)N(C1CCCC1)CC(F)(F)C(=O)N2C ; O=C1CCC(Nc2cc(F)c(N3CCNCC3)cc2F)C(=O)N1 ; CC(C)(C)OC(=O)N1CCC(CC=O)CC1 ; CC(C)(C)OC(=O)N1CCC(CCN2CCN(c3cc(F)c(NC4CCC(=O)NC4=O)cc3F)CC2)CC1 ; O=C1CCC(Nc2cc(F)c(N3CCN(CCC4CCNCC4)CC3)cc2F)C(=O)N1]

### Step 1. Synthesis of tert-butyl 2-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)hydrazine-1-carboxylate (2)

It was synthesized by similar method described in step 10 of Example 2 to obtain tert-butyl 2-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)hydrazine-1-carboxylate (7.6 g, 13.40 mmol, 99.91% yield, 99% purity) as a yellow solid. MS(M+H)⁺=562.3

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzohydrazide (3)

It was synthesized by similar method described in step 6 of Example 3 to obtain 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzohydrazide (3 g, 6.50 mmol, 48.04% yield, 100% purity) as a yellow solid. MS(M+H)⁺=462.2

### Step 3. Synthesis of N′-(2-chloroacetyl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzohydrazide (5)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzohydrazide (2.5 g, 5.42 mmol) in DCM (100 mL) was added DIPEA (1.39 g, 10.74 mmol, 1.87 mL) at 0° C., then a solution of 2-chloroacetyl chloride (490 mg, 4.34 mmol, 345.56 μL) in DCM (5 mL) was added slowly at 0° C. and the mixture was stirred at 0° C. for 0.5 h. LCMS showed 21% of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzohydrazide remained and 72% of the desired mass. Then a solution of 2-chloroacetyl chloride (95 mg, 841.14 μmol, 67.00 μL) in DCM (1 mL) was added and the mixture was stirred at 0° C. for another 0.5 h. LCMS showed 8% of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzohydrazide remained and 84% of the desired mass. Then a solution of 2-chloroacetyl chloride (110 mg, 973.95 μmol, 77.57 μL) in DCM (1 mL) was added and the mixture was stirred at 0° C. for additional 0.5 h. The mixture was diluted with water (50 mL) and extracted with DCM (20 mL). The organic layer was washed with water (20 mL), dried over Na₂SO₄ and filtered. The filtrate was concentrated under reduced pressure to give a residue, which was purified by flash silica gel chromatography (20 g SepaFlash® Silica Flash Column, Eluent of 80-100% EtOAc/Petroleum ether gradient @ 100 mL/min) to afford N′-(2-chloroacetyl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzohydrazide (2.5 g, 4.23 mmol, 78.06% yield, 91% purity) as a yellow solid. MS(M+H)⁺=538.2

### Step 4. Synthesis of 2-((4-(5-(chloromethyl)-1,3,4-oxadiazol-2-yl)-2-methoxyphenyl)amino)-9-cyclopentyl-7,7-difluoro-5-methyl-5,7,8,9-tetrahydro-6H-pyrimido[4,5-b][1,4]diazepin-6-one (6)

To a solution of N′-(2-chloroacetyl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzohydrazide (2.4 g, 4.46 mmol) in toluene (50 mL) was added POCl₃ (13.16 g, 85.83 mmol, 8 mL) slowly at 25° C. and the mixture was stirred at 80° C. under N₂ for 14 h. LCMS showed the starting material remained. Additional POCl₃ (13.16 g, 85.83 mmol, 8 mL) was added at 25° C. and the mixture was stirred at 80° C. under N₂ for another 38 h. The suspension clarified. The mixture was combined with another batch (0.1 g scale), then concentrated under reduced pressure. The crude was diluted with DCM (100 mL), then poured into saturated NaHCO₃ solution (100 mL) slowly. The mixture was extracted with EtOAc (50 mL×3). The combined organic layer was washed with saturated NaHCO₃ solution (20 mL) and water (50 mL), dried over Na₂SO₄ and filtered. The filtrate was concentrated under reduced pressure to afford 2-((4-(5-(chloromethyl)-1,3,4-oxadiazol-2-yl)-2-methoxyphenyl)amino)-9-cyclopentyl-7,7-difluoro-5-methyl-5,7,8,9-tetrahydro-6H-pyrimido[4,5-b][1,4]diazepin-6-one (2.16 g, crude) as a red solid. MS(M+H)⁺=520.2

### Step 5. Synthesis of tert-butyl 4-(2-(4-(4-((2,6-dioxopiperidin-3-yl)amino)-2,5-difluorophenyl)piperazin-1-yl)ethylpiperidine-1-carboxylate (7C)

It was synthesized by similar method described in step 5 of Example 56 to obtain tert-butyl 4-(2-(4-(4-((2,6-dioxopiperidin-3-yl)amino)-2,5-difluorophenyl)piperazin-1-yl)ethyl)piperidine-1-carboxylate (8.6 g, 16.06 mmol, 57.93% yield, 100% purity) as a gray solid. MS(M+H)⁺=536.5

### Step 6. Synthesis of 3-((2,5-difluoro-4-(4-(2-(piperidin-4-yl)ethyl)piperazin-1-yl)phenyl)amino)piperidine-2,6-dione (7)

It was synthesized by similar method described in step 2 of Example 48 to obtain 3-((2,5-difluoro-4-(4-(2-(piperidin-4-yl)ethyl)piperazin-1-yl)phenyl)amino)piperidine-2,6-dione (8 g, crude, HCl salt) as a gray solid. MS(M+H)⁺=436.3

### Step 7. Synthesis of 3-((4-(4-(2-(1-((5-(4-((9-cyclopentyl-7,7-difluoro-5-methyl-6-oxo-6,7,8,9-tetrahydro-5H-pyrimido[4,5-b][1,4]diazepin-2-yl)amino)-3-methoxyphenyl)-1,3,4-oxadiazol-2-yl)methyl)piperidin-4-yl)ethyl)piperazin-1-yl)-2,5-difluorophenyl)amino)piperidine-2,6-dione (Compound 65)

It was synthesized by similar method described in step 8 of Example 25 to obtain two batches of product. Batch 1: 3-((4-(4-(2-(1-((5-(4-((9-cyclopentyl-7,7-difluoro-5-methyl-6-oxo-6,7,8,9-tetrahydro-5 H-pyrimido[4,5-b][1,4]diazepin-2-yl)amino)-3-methoxyphenyl)-1,3,4-oxadiazol-2-yl) methyl)piperidin-4-yl)ethyl)piperazin-1-yl)-2,5-difluorophenyl)amino)piperidine-2,6-dione (1.81 g, 1.96 mmol, 59.91% yield, 99.3% purity) as a yellow solid. Batch 2: 3-((4-(4-(2-(1-((5-(4-((9-cyclopentyl-7,7-difluoro-5-methyl-6-oxo-6,7,8,9-tetrahydro-5 H-pyrimido[4,5-b][1,4]diazepin-2-yl)amino)-3-methoxyphenyl)-1,3,4-oxadiazol-2-yl) methyl)piperidin-4-yl)ethyl)piperazin-1-yl)-2,5-difluorophenyl)amino)piperidine-2,6-dione (628 mg, 670.38 mol, 20.54% yield, 98% purity) as a yellow solid. MS(M+H)⁺=919.7

¹H NMR (400 MHz, DMSO-d₆) δ=10.78 (s, 1H), 8.43 (d, J=8.4 Hz, 1H), 8.28 (s, 1H), 8.11 (s, 1H), 7.60-7.49 (m, 2H), 6.81 (br dd, J=7.8, 13.2 Hz, 1H), 6.71 (br dd, J=8.1, 14.7 Hz, 1H), 5.49-5.44 (m, 1H), 4.83-4.72 (m, 1H), 4.37-4.27 (m, 1H), 4.04 (br t, J=13.8 Hz, 2H), 3.98 (s, 3H), 3.82 (s, 2H), 3.37 (s, 3H), 2.90-2.78 (m, 6H), 2.76-2.65 (m, 1H), 2.58-2.52 (m, 1H), 2.47-2.41 (m, 3H), 2.33-2.26 (m, 2H), 2.14-2.05 (m, 2H), 2.04-1.89 (m, 4H), 1.72-1.53 (m, 9H), 1.39-1.31 (m, 2H), 1.28-1.10 (m, 3H).

### Example 66. Synthesis of 3-((4-(4-(2-(1-(5-(4-((9-cyclopentyl-7,7-difluoro-5-methyl-6-oxo-6,7,8,9-tetrahydro-5H-pyrimido[4,5-b][1,4]diazepin-2-yl)amino)-3-methoxyphenyl)-1,3,4-oxadiazol-2-yl)piperidin-4-yl)ethyl)piperazin-1-yl)-2,5-difluorophenyl)amino)piperidine-2,6-dione (Compound 66)

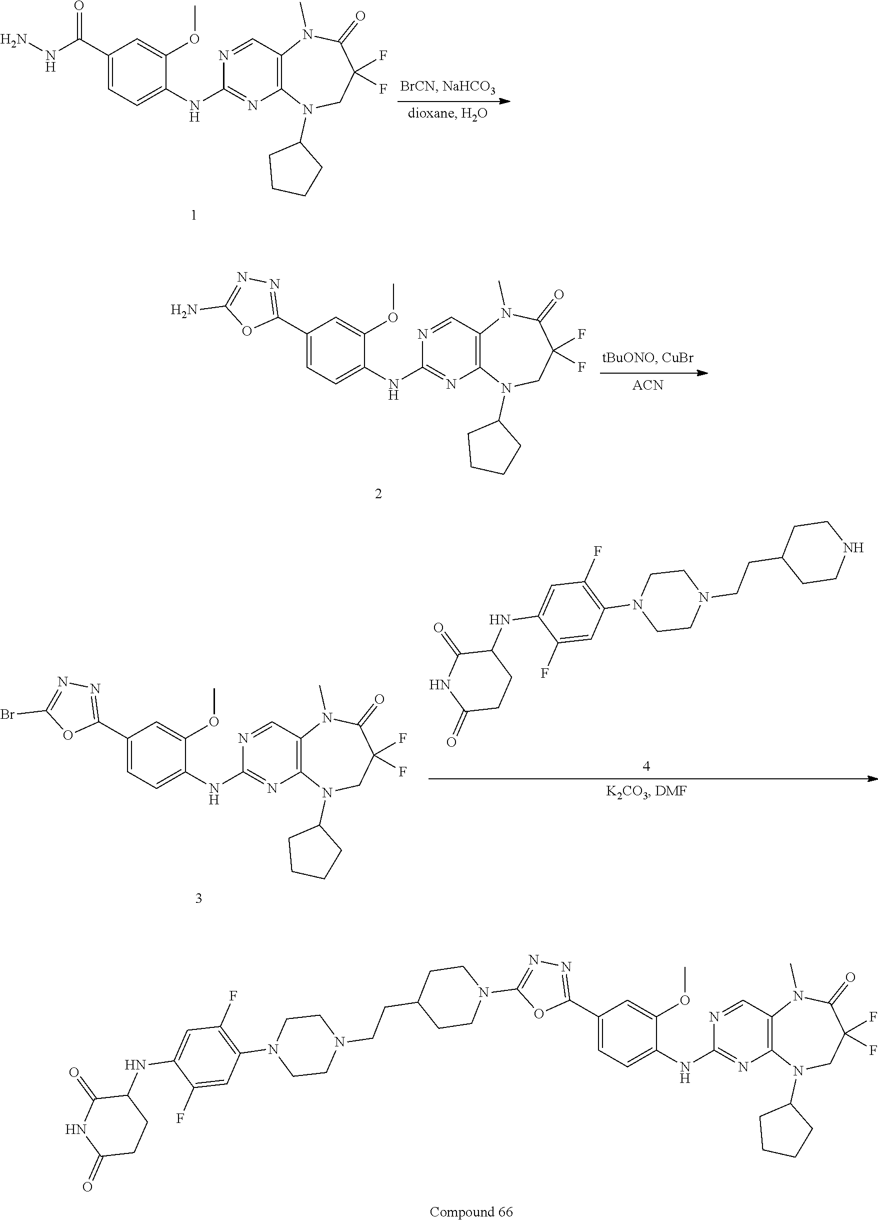

### Step 1. Synthesis of 2-((4-(5-amino-1,3,4-oxadiazol-2-yl)-2-methoxyphenyl)amino)-9-cyclopentyl-7,7-difluoro-5-methyl-5,7,8,9-tetrahydro-6H-pyrimido[4,5-b][1,4]diazepin-6-one (2)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzohydrazide (2.1 g, 4.55 mmol) in dioxane (40 mL) was added a solution of NaHCO₃ (1.15 g, 13.66 mmol, 531.45 μL) in H₂O (20 mL), then BrCN (1.05 g, 9.91 mmol, 727.65 μL) was added and the mixture was stirred at 25° C. for 13 h under N₂. The mixture was filtered and the filter cake was washed with MTBE (30 mL) and water (30 mL). The filter cake was collected and dried under reduced pressure to afford 2-((4-(5-amino-1,3,4-oxadiazol-2-yl)-2-methoxyphenyl)amino)-9-cyclopentyl-7,7-difluoro-5-methyl-5,7,8,9-tetrahydro-6H-pyrimido[4,5-b][1,4]diazepin-6-one (1.81 g, 3.53 mmol, 77.67% yield, 95% purity) as a yellow solid. The filtrate was extracted with EtOAc (30 mL×3). The combined organic layer was washed with brine (10 mL×2), dried over Na₂SO₄ and filtered. The filtrate was concentrated under reduced pressure to give a residue, which was diluted with MTBE (10 mL) and stirred at 25° C. for 0.5 h. The suspension was filtered and the filter cake was washed with MTBE (10 mL) and water (10 mL). The filter cake was collected and dried under reduced pressure to afford 2-((4-(5-amino-1,3,4-oxadiazol-2-yl)-2-methoxyphenyl)amino)-9-cyclopentyl-7,7-difluoro-5-methyl-5,7,8,9-tetrahydro-6H-pyrimido[4,5-b][1,4]diazepin-6-one (380 mg, crude) as a yellow solid. MS(M+H)⁺=487.3

### Step 2. Synthesis of 2-((4-(5-bromo-1,3,4-oxadiazol-2-yl)-2-methoxyphenyl)amino)-9-cyclopentyl-7,7-difluoro-5-methyl-5,7,8,9-tetrahydro-6H-pyrimido[4,5-b][1,4]diazepin-6-one (3)

To a solution of CuBr (1.80 g, 12.55 mmol, 382.17 μL) in CAN (20 mL) was added t-BuONO (1.28 g, 12.41 mmol, 1.48 mL), then a solution of 2-((4-(5-amino-1,3,4-oxadiazol-2-yl)-2-methoxyphenyl)amino)-9-cyclopentyl-7,7-difluoro-5-methyl-5,7,8,9-tetrahydro-6H-pyrimido[4,5-b][1,4]diazepin-6-one (2 g, 4.11 mmol) in CAN (90 mL) was added and the resulting mixture was stirred at 60° C. for 1 h. LCMS showed the desired mass. The mixture was cooled to 25° C., then adjusted the pH-8 with saturated NaHCO₃. The mixture was filtered and the filter cake was washed with THF (100 mL) and EtOAc (50 mL). The filtrate was concentrated under reduced pressure. The crude was diluted with water (30 mL), then extracted with EtOAc (10 mL×3). The combined organic layer was washed with brine (20 mL), dried over Na₂ SO₄ and filtered. The filtrate was concentrated under reduced pressure to give a residue, which was purified by flash silica gel chromatography (12 g SepaFlash® Silica Flash Column, Eluent of 0-10% MeOH/EtOAc gradient @ 100 mL/min) to afford 2-((4-(5-bromo-1,3,4-oxadiazol-2-yl)-2-methoxyphenyl)amino)-9-cyclopentyl-7,7-difluoro-5-methyl-5,7,8,9-tetrahydro-6H-pyrimido[4,5-b][1,4]diazepin-6-one (0.6 g, 1.09 mmol, 26.52% yield) as a yellow solid. MS(M+H)⁺=550.3

### Step 3. Synthesis of 3-((4-(4-(2-(1-(5-(4-((9-cyclopentyl-7,7-difluoro-5-methyl-6-oxo-6,7,8,9-tetrahydro-5H-pyrimido[4,5-b][1,4]diazepin-2-yl)amino)-3-methoxyphenyl)-1,3,4-oxadiazol-2-yl)piperidin-4-yl)ethyl)piperazin-1-yl)-2,5-difluorophenyl)amino)piperidine-2,6-dione (Compound 66)

To a solution of 3-((2,5-difluoro-4-(4-(2-(piperidin-4-yl)ethyl)piperazin-1-yl)phenyl)amino)piperidine-2,6-dione (1 g, 2.12 mmol, HCl salt) and K₂CO₃ (603.33 mg, 4.37 mmol, 279.45 μL) in DMF (10 mL) was added 2-((4-(5-bromo-1,3,4-oxadiazol-2-yl)-2-methoxyphenyl)amino)-9-cyclopentyl-7,7-difluoro-5-methyl-5,7,8,9-tetrahydro-6H-pyrimido[4,5-b][1,4]diazepin-6-one (0.6 g, 1.09 mmol) and the resulting mixture was stirred at 50° C. for 14 h. LCMS showed a peak (82%) with desired mass. The mixture was diluted with EtOAc (10 mL) and water (30 mL). The mixture was extracted with EtOAc (10 mL×3). The combined organic layer was washed with brine (20 mL), dried over Na₂SO₄ and filtered. The filtrate was concentrated under reduced pressure. The residue was purified by flash silica gel chromatography (5 g SepaFlash® Silica Flash Column, Eluent of 10-20% MeOH/DCM gradient @ 50 mL/min) followed by prep-TLC (EtOAc/MeOH=10/1). The impure product was re-purified by prep-HPLC (column: Phenomenex luna C18 150*40 mm*15 μm; mobile phase: [water (TFA)-ACN]; gradient: 25%-55% B over 10 min) and the eluent was lyophilized to afford 3-((4-(4-(2-(1-(5-(4-((9-cyclopentyl-7,7-difluoro-5-methyl-6-oxo-6,7,8,9-tetrahydro-5 H-pyrimido[4,5-b][1,4]diazepin-2-yl)amino)-3-methoxyphenyl)-1,3,4-oxadiazol-2-yl)piperidin-4-yl)ethyl)piperazin-1-yl)-2,5-difluorophenyl)amino)piperidine-2,6-dione (568.5 mg, 544.52 mol, 49.95% yield, 97.6% purity, TFA) as a blue solid. MS(M+H)⁺=905.6

¹H NMR (400 MHz, CD₃OD) δ=8.33 (d, J=8.4 Hz, 1H), 8.16 (s, 1H), 7.58-7.56 (m, 1H), 7.52 (dd, J=1.8, 8.5 Hz, 1H), 6.89 (dd, J=7.7, 12.6 Hz, 1H), 6.72 (dd, J=7.9, 13.8 Hz, 1H), 4.99-4.93 (m, 1H), 4.31 (dd, J=5.0, 12.4 Hz, 1H), 4.14-4.05 (m, 4H), 4.03 (s, 3H), 3.66 (br d, J=12.4 Hz, 2H), 3.46-3.39 (m, 5H), 3.30-3.24 (m, 4H), 3.22-3.14 (m, 2H), 3.11-3.02 (m, 2H), 2.87-2.78 (m, 1H), 2.75-2.68 (m, 1H), 2.33-2.25 (m, 1H), 2.10-1.99 (m, 3H), 1.94-1.87 (m, 2H), 1.84-1.75 (m, 4H), 1.74-1.64 (m, 5H), 1.50-1.38 (m, 2H).

### Example 67. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2,5-difluorophenyl)piperazin-1-yl)ethyl)piperidin-4-yl)-3-methoxybenzamide (Compound 67)

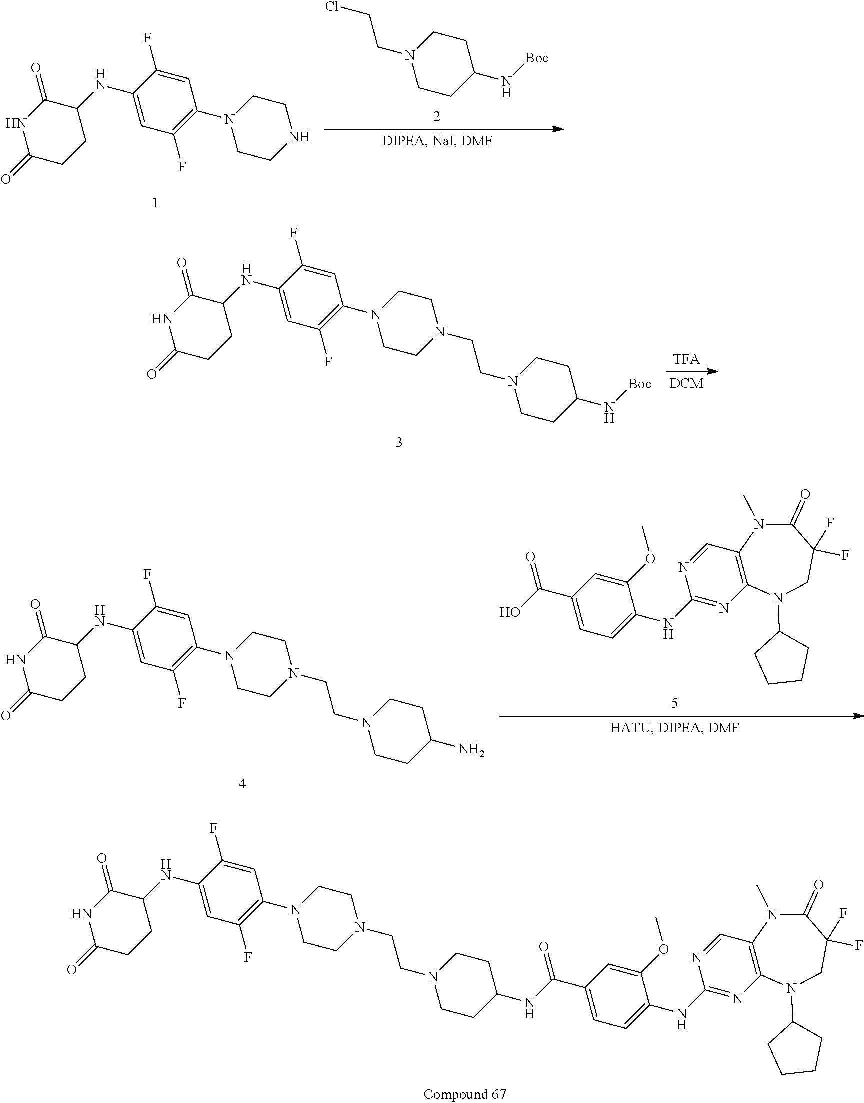 [SMILES: O=C1CCC(Nc2cc(F)c(N3CCNCC3)cc2F)C(=O)N1 ; CC(C)(C)OC(=O)NC1CCN(CCCl)CC1 ; CC(C)(C)OC(=O)NC1CCN(CCN2CCN(c3cc(F)c(NC4CCC(=O)NC4=O)cc3F)CC2)CC1 ; NC1CCN(CCN2CCN(c3cc(F)c(NC4CCC(=O)NC4=O)cc3F)CC2)CC1 ; COc1cc(C(=O)O)ccc1Nc1ncc2c(n1)N(C1CCCC1)CC(F)(F)C(=O)N2C ; COc1cc(C(=O)NC2CCN(CCN3CCN(c4cc(F)c(NC5CCC(=O)NC5=O)cc4F)CC3)CC2)ccc1Nc1ncc2c(n1)N(C1CCCC1)CC(F)(F)C(=O)N2C]

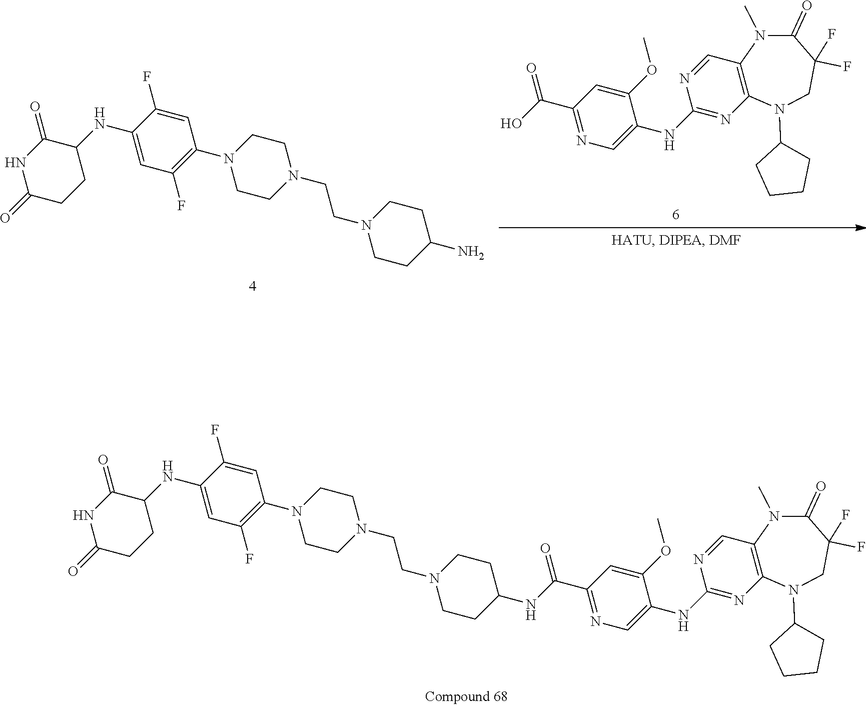 [SMILES: NC1CCN(CCN2CCN(c3cc(F)c(NC4CCC(=O)NC4=O)cc3F)CC2)CC1 ; COc1cc(C(=O)O)ncc1Nc1ncc2c(n1)N(C1CCCC1)CC(F)(F)C(=O)N2C ; COc1cc(C(=O)NC2CCN(CCN3CCN(c4cc(F)c(NC5CCC(=O)NC5=O)cc4F)CC3)CC2)ncc1Nc1ncc2c(n1)N(C1CCCC1)CC(F)(F)C(=O)N2C]

### Step 1. Synthesis of tert-butyl (1-(2-(4-(4-((2,6-dioxopiperidin-3-yl)amino)-2,5-difluorophenyl)piperazin-1-yl)eth yl)piperidin-4-yl)carbamate (3)

It was synthesized by similar method described in step 3 of Example 23 to obtain tert-butyl (1-(2-(4-(4-((2,6-dioxopiperidin-3-yl)amino)-2,5-difluorophenyl)piperazin-1-yl)ethyl) piperidin-4-yl)carbamate (4 g, 7.12 mmol, 39.51% yield, 98% purity) as a white solid. MS(M+H)⁺=551.5.

### Step 2. Synthesis of 3-((4-(4-(2-(4-aminopiperidin-1-yl)ethyl)piperazin-1-yl)-2,5-difluorophenyl)amino)piperidine-2,6-dione (4)

It was synthesized by similar method described in step 3 of Example 3 to obtain 3-((4-(4-(2-(4-aminopiperidin-1-yl)ethyl)piperazin-1-yl)-2,5-difluorophenyl)amino)piperidine-2,6-dione (3 g, 5.31 mmol, 97.54% yield, TFA salt) as yellow oil. MS(M+H)⁺=451.3.

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2,5-difluorophenyl)piperazin-1-yl)ethyl)piperidin-4-yl)-3-methoxybenzamide (Compound 67)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2,5-difluoro phenyl)piperazin-1-yl)ethyl)piperidin-4-yl)-3-methoxybenzamide (1.81 g, 2.04 mmol, 38.37% yield, 99% purity) as a white solid. MS(M+H)⁺=880.4.

¹H NMR (400 MHz, CD₃OD) δ=8.50 (d, J=8.2 Hz, 1H), 8.20 (s, 1H), 7.60-7.49 (m, 2H), 6.85 (dd, J=7.7, 13.0 Hz, 1H), 6.78-6.64 (m, 1H), 4.30 (dd, J=4.8, 12.5 Hz, 1H), 4.20-4.09 (m, 1H), 4.08-3.98 (m, 5H), 3.61-3.49 (m, 1H), 3.42 (s, 3H), 3.24-3.18 (m, 2H), 3.12-3.00 (m, 6H), 2.93-2.72 (m, 9H), 2.36-2.29 (m, 1H), 2.26-2.17 (m, 2H), 2.14-2.06 (m, 2H), 2.05-1.92 (m, 3H), 1.88-1.81 (m, 2H), 1.79-1.59 (m, 5H).

### Example 68. Synthesis of 5-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2,5-difluorophenyl)piperazin-1-yl)ethyl)piperidin-4-yl)-4-methoxypicolinamide (Compound 68)

It was synthesized by similar method described in step 10 of Example 2 to obtain 5-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(4-((2,6-dioxopiperidin-3-yl)amino)-2,5-difluoro phenyl)piperazin-1-yl)ethyl)piperidin-4-yl)-4-methoxypicolinamide (0.7637 g, 858.25 mol, 40.38% yield, 99% purity) as a yellow solid. MS(M+H)⁺=881.4.

¹H NMR (400 MHz, DMSO-d₆) δ=10.81 (s, 1H), 9.05 (s, 1H), 8.81 (br d, J=7.9 Hz, 1H), 8.62 (br s, 1H), 8.24 (s, 1H), 7.71 (s, 1H), 6.97 (dd, J=8.0, 12.9 Hz, 1H), 6.79 (dd, J=8.2, 14.4 Hz, 1H), 5.84-5.59 (m, 1H), 4.73-4.61 (m, 1H), 4.39 (br t, J=8.6 Hz, 1H), 4.22-3.91 (m, 6H), 3.64-3.43 (m, 6H), 3.43-3.28 (m, 6H), 3.28-2.92 (m, 7H), 2.79-2.66 (m, 1H), 2.62-2.54 (m, 1H), 2.12-1.92 (m, 6H), 1.89-1.81 (m, 2H), 1.66 (br s, 2H), 1.61-1.47 (m, 4H).

### Example 69. Synthesis of 4-((7,7-difluoro-9-isopropyl-5-methyl-6-oxo-6,7,8,9-tetrahydro-5H-pyrimido[4,5-b ][1,4]diazepin-2-yl)amino)-N-(7-((1-(4-((2,6-dioxopiperidin-3-yl)amino)-2,5-difluorophenyl)piperidin-4-yl)methyl)-7-azaspiro[3.5]nonan-2-yl)-2-fluoro-5-methoxybenzamide (Compound 69)

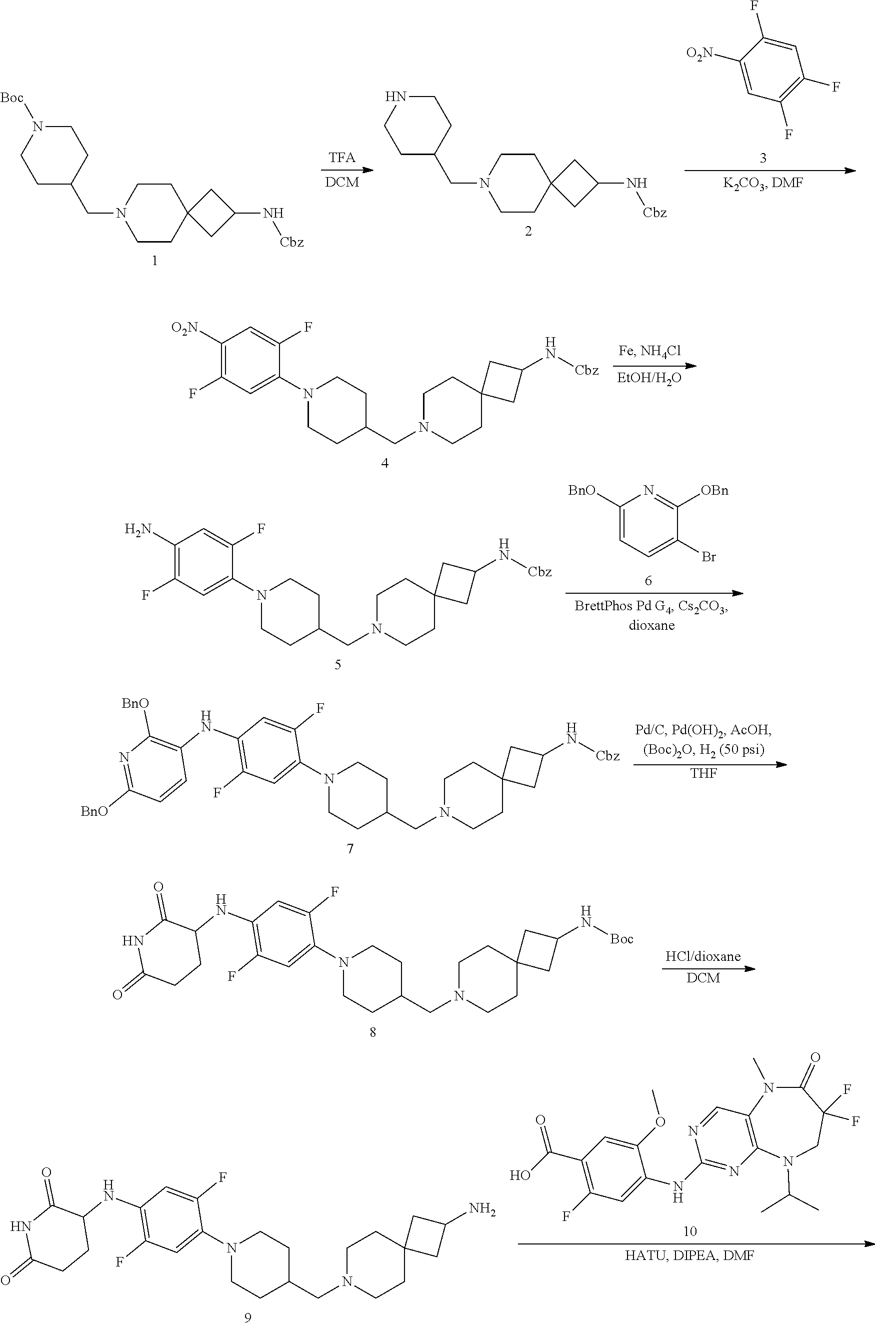

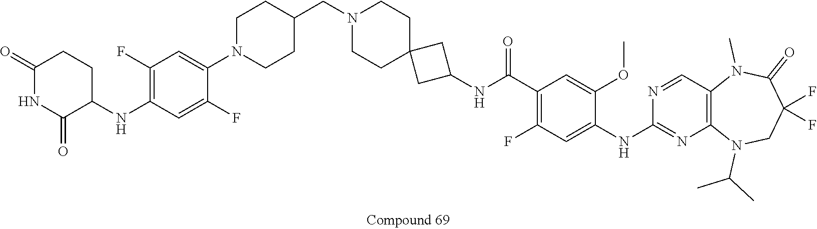 [SMILES: COc1cc(C(=O)NC2CC3(CCN(CC4CCN(c5cc(F)c(NC6CCC(=O)NC6=O)cc5F)CC4)CC3)C2)c(F)cc1Nc1ncc2c(n1)N(C(C)C)CC(F)(F)C(=O)N2C]

### Step 1. Synthesis of benzyl (7-(piperidin-4-ylmethyl)-7-azaspiro[3.5]nonan-2-yl)carbamate (2)

It was synthesized by similar method described in step 6 of Example 3 to obtain benzyl (7-(piperidin-4-ylmethyl)-7-azaspiro[3.5]nonan-2-yl)carbamate (23 g, crude, TFA salt) as a colorless oil. MS(M+H)⁺=372.2.

### Step 2. Synthesis of benzyl (7-((1-(2,5-difluoro-4-nitrophenyl)piperidin-4-yl)methyl)-7-azaspiro[3.5]nonan-2-yl)carbamate (4)

It was synthesized by similar method described in step 3 of Example 62 to obtain benzyl (7-((1-(2,5-difluoro-4-nitrophenyl)piperidin-4-yl)methyl)-7-azaspiro[3.5]nonan-2-yl)carbamate (20 g, 37.84 mmol, 79.87% yield) as a yellow solid. MS(M+H)⁺=529.3.

### Step 3. Synthesis of benzyl (7-((1-(4-amino-2,5-difluorophenyl)piperidin-4-yl)methyl)-7-azaspiro[3.5]nonan-2-yl)carbamate (5)

It was synthesized by similar method described in step 4 of Example 62 to obtain benzyl (7-((1-(4-amino-2,5-difluorophenyl)piperidin-4-yl)methyl)-7-azaspiro[3.5]nonan-2-yl) carbamate (20 g) as a yellow solid. MS(M+H)⁺=499.3.

### Step 4. Synthesis of benzyl (7-((1-(4-((2,6-bis(benzyloxy)pyridin-3-yl)amino)-2,5-difluorophenyl)piperidin-4-yl)methyl)-7-azaspiro[3.5]nonan-2-yl)carbamate (7)

It was synthesized by similar method described in step 5 of Example 62 to obtain benzyl (7-((1-(4-((2,6-bis(benzyloxy)pyridin-3-yl)amino)-2,5-difluorophenyl)piperidin-4-yl)methyl)-7-azaspiro[3.5]nonan-2-yl)carbamate (16 g, 20.31 mmol, 72.32% yield) as a brown oil. MS(M+H)⁺=788.4.

### Step 5. Synthesis of tert-butyl (7-((1-(4-((2,6-dioxopiperidin-3-yl)amino)-2,5-difluorophenyl)piperidin-4-yl)meth yl)-7-azaspiro[3.5]nonan-2-yl)carbamate (8)

It was synthesized by similar method described in step 6 of Example 62 to obtain tert-butyl (7-((1-(4-((2,6-dioxopiperidin-3-yl)amino)-2,5-difluorophenyl)piperidin-4-yl)methyl)-7-azaspiro[3.5]nonan-2-yl)carbamate (2 g) as a blue solid. MS(M+H)⁺=576.3.

### Step 6. Synthesis of 3-((4-(4-((2-amino-7-azaspiro[3.5]nonan-7-yl)methyl)piperidin-1-yl)-2,5-difluorophenyl)amino)piperidine-2,6-dione (9)

It was synthesized by similar method described in step 2 of Example 48 to obtain 3-((4-(4-((2-amino-7-azaspiro[3.5]nonan-7-yl)methyl)piperidin-1-yl)-2,5-difluorophenyl)amino)piperidine-2,6-dione (1.78 g, 3.48 mmol, 100.00% yield, HCl salt) as a blue solid. MS(M+H)⁺=476.2.

### Step 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2,5-difluorophenyl)piperidin-4-yl)methyl)-7-azaspiro[3.5]nonan-2-yl)-2-fluoro-5-methoxybenzamide (Compound 69)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2,5-difluorophenyl)piperidin-4-yl)methyl)-7-azaspiro[3.5]nonan-2-yl)-2-fluoro-5-methoxybenzamide (233.6 mg, 252.63 μmol, 22.66% yield, 97% purity) as a yellow solid. MS(M+H)⁺=897.2.

¹H NMR (400 MHz, DMSO-d₆) δ=10.80 (s, 1H), 8.36-8.21 (m, 3H), 7.98 (s, 1H), 7.21 (d, J=6.6 Hz, 1H), 6.90-6.79 (m, 1H), 6.78-6.67 (m, 1H), 5.50 (d, J=8.0 Hz, 1H), 4.95-4.86 (m, 1H), 4.49-4.30 (m, 2H), 4.07 (t, J=13.4 Hz, 2H), 3.92 (s, 3H), 3.33-3.32 (m, 3H), 3.18-3.13 (m, 2H), 3.06-2.84 (m, 2H), 2.79-2.65 (m, 2H), 2.63-2.52 (m, 5H), 2.35-2.10 (m, 3H), 2.09-1.96 (m, 3H), 1.95-1.59 (m, 9H), 1.47-1.22 (m, 8H).

### Example 70. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2,5-difluorophenyl)piperidin-4-yl)methyl)-7-azaspiro[3.5]nonan-2-yl)-3-methoxybenzamide (Compound 70)

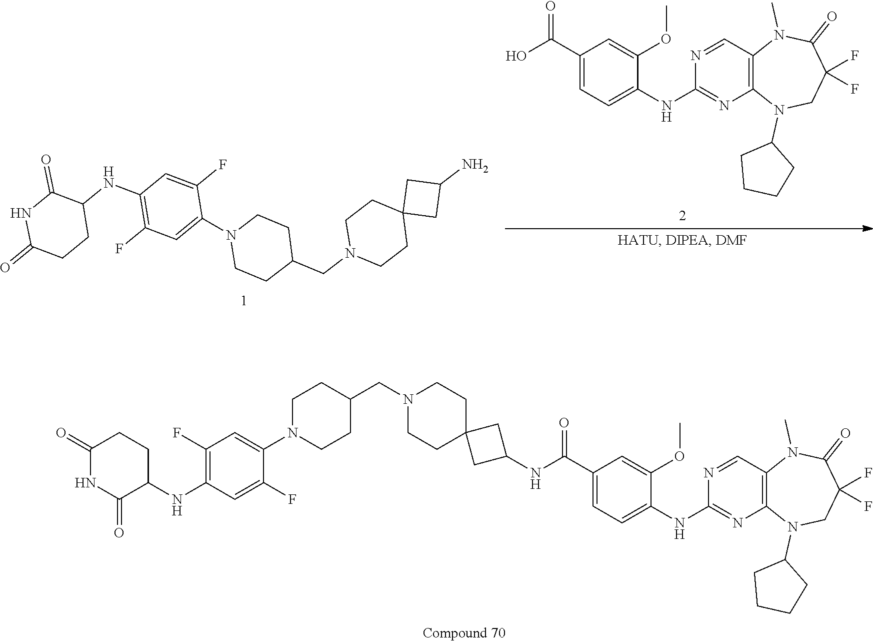 [SMILES: NC1CC2(CCN(CC3CCN(c4cc(F)c(NC5CCC(=O)NC5=O)cc4F)CC3)CC2)C1 ; COc1cc(C(=O)O)ccc1Nc1ncc2c(n1)N(C1CCCC1)CC(F)(F)C(=O)N2C ; COc1cc(C(=O)NC2CC3(CCN(CC4CCN(c5cc(F)c(NC6CCC(=O)NC6=O)cc5F)CC4)CC3)C2)ccc1Nc1ncc2c(n1)N(C1CCCC1)CC(F)(F)C(=O)N2C]

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(4-((2,6-dioxopiperidin-3-yl)amino)-2,5-difluorophenyl)piperidin-4-yl)methyl)-7-azaspiro[3.5]nonan-2-yl)-3-methoxybenzamide (274 mg, 296.71 μmol, 11.69% yield, 98% purity) as a white solid. MS(M+H)⁺=905.5.

¹H NMR (400 MHz, DMSO-d₆) δ=10.79 (s, 1H), 8.42 (br d, J=6.9 Hz, 1H), 8.30-8.23 (m, 2H), 7.96 (s, 1H), 7.51-7.44 (m, 2H), 6.87-6.66 (m, 2H), 5.46 (br d, J=8.6 Hz, 1H), 4.82-4.71 (m, 1H), 4.46-4.28 (m, 2H), 4.04 (br t, J=13.9 Hz, 2H), 3.94 (s, 3H), 3.31 (s, 3H), 3.18-3.08 (m, 2H), 2.80-2.66 (m, 1H), 2.61-2.52 (m, 3H), 2.36-2.09 (m, 7H), 2.08-1.90 (m, 5H), 1.87-1.68 (m, 6H), 1.66-1.48 (m, 9H), 1.31-1.14 (m, 2H).

### Example 71. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,3-difluorophenyl)piperazin-1-yl)ethyl)piperidin-1-yl)-3-methoxybenzamide (Compound 71)

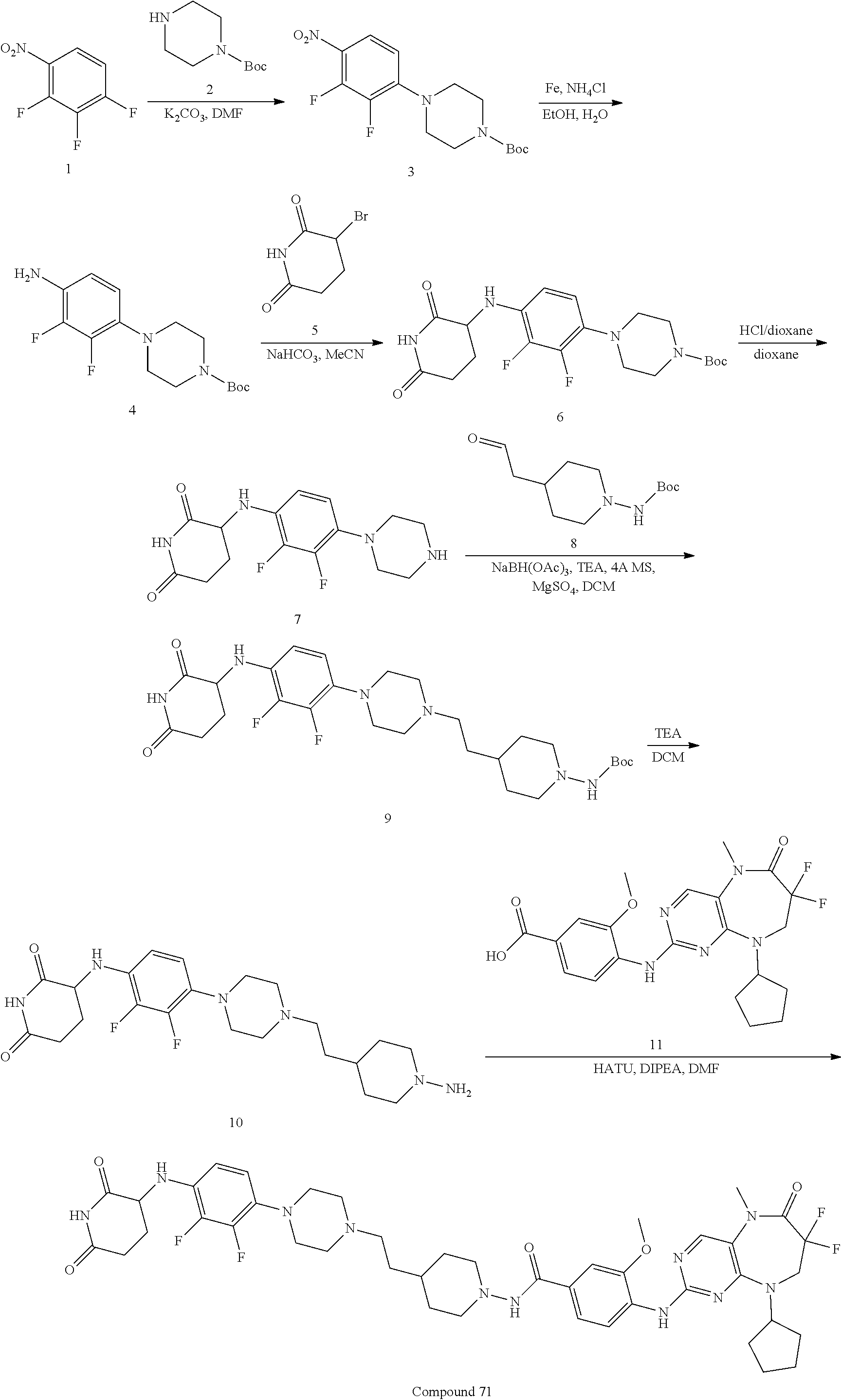

### Step 1. Synthesis of tert-butyl 4-(2,3-difluoro-4-nitrophenyl)piperazine-1-carboxylate (3)

It was synthesized by similar method described in step 4 of Example 30 to obtain tert-butyl 4-(2,3-difluoro-4-nitrophenyl)piperazine-1-carboxylate (4.8 g, 13.56 mmol, 48.03% yield, 97% purity) as a white solid. MS(M+H-55)⁺=288.0.

¹H NMR (400 MHz, CDCl₃) δ=7.91-7.77 (m, 1H), 6.66 (br t, J=8.4 Hz, 1H), 3.61-3.59 (m, 4H), 3.30-3.28 (m, 4H), 1.49-1.46 (m, 9H).

### Step 2. Synthesis of tert-butyl 4-(4-amino-2,3-difluorophenyl)piperazine-1-carboxylate (4)

It was synthesized by similar method described in step 4 of Example 22 to obtain tert-butyl 4-(4-amino-2,3-difluorophenyl)piperazine-1-carboxylate (4.6 g, 12.48 mmol, 89.25% yield, 85% purity) as a brown solid. MS(M+H)⁺=314.0.

### Step 3. Synthesis of tert-butyl 4-(4-((2,6-dioxopiperidin-3-yl)amino)-2,3-difluorophenyl)piperazine-1-carboxylate (6)

It was synthesized by similar method described in step 5 of Example 22 to obtain tert-butyl 4-(4-((2,6-dioxopiperidin-3-yl)amino)-2,3-difluorophenyl)piperazine-1-carboxylate (2.7 g, 5.85 mmol, 39.87% yield, 92% purity) as a blue solid. MS(M+H)⁺=425.2.

¹H NMR (400 MHz, CDCl₃) δ=8.37 (br s, 1H), 6.61 (br t, J=7.9 Hz, 1H), 6.40 (br t, J=8.1 Hz, 1H), 4.75-4.57 (m, 1H), 4.10-4.06 (m, 1H), 3.59 (br d, J=4.4 Hz, 4H), 2.93 (br d, J=4.4 Hz, 4H), 2.94-2.76 (m, 1H), 2.65-2.27 (m, 2H), 2.05-1.91 (m, 1H), 1.50 (s, 9H).

### Step 4. Synthesis of 3-((2,3-difluoro-4-(piperazin-1-yl)phenyl)amino)piperidine-2,6-dione (7)

It was synthesized by similar method described in step 5 of Example 1 to obtain 3-((2,3-difluoro-4-(piperazin-1-yl)phenyl)amino)piperidine-2,6-dione (2.3 g, crude, HCl salt) as a blue solid. MS(M+H)⁺=325.1.

### Step 5. Synthesis of tert-butyl (4-(2-(4-(4-((2,6-dioxopiperidin-3-yl)amino)-2,3-difluorophenyl)piperazin-1-yl)eth yl)piperidin-1-yl)carbamate (9)

It was synthesized by similar method described in step 6 of Example 1 to obtain tert-butyl (4-(2-(4-(4-((2,6-dioxopiperidin-3-yl)amino)-2,3-difluorophenyl)piperazin-1-yl)ethyl)piperidin-1-yl)carbamate (1.05 g, 1.72 mmol, 26.92% yield, 90% purity) as a yellow solid. MS(M+H)⁺=551.3.

### Step 6. Synthesis of 3-((4-(4-(2-(1-aminopiperidin-4-yl)ethyl)piperazin-1-yl)-2,3-difluorophenyl)amino)piperidine-2,6-dione (10)

It was synthesized by similar method described in step 6 of Example 3 to obtain 3-((4-(4-(2-(1-aminopiperidin-4-yl)ethyl)piperazin-1-yl)-2,3-difluorophenyl)amino)piperidine-2,6-dione (1.08 g, crude, TFA salt) as brown oil. MS(M+H)⁺=451.2.

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,3-difluorophenyl)piperazin-1-yl)ethyl)piperidin-1-yl)-3-methoxybenzamide (Compound 71)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2,3-difluoro phenyl)piperazin-1-yl)ethyl)piperidin-1-yl)-3-methoxybenzamide (899 mg, 980.79 mol, 51.27% yield, 96% purity) as a white solid. MS(M+H)⁺=880.4.

¹H NMR (400 MHz, DMSO-d₆) δ=10.82 (s, 1H), 9.90-9.56 (m, 2H), 8.34-8.13 (m, 3H), 7.54-7.37 (m, 2H), 6.80-6.68 (m, 1H), 6.66-6.53 (m, 1H), 4.81-4.76 (m, 1H), 4.43-4.38 (m, 1H), 4.08 (br t, J=13.6 Hz, 2H), 3.94 (s, 3H), 3.60-3.52 (m, 2H), 3.40-3.29 (m, 5H), 3.28-3.09 (m, 6H), 3.07-2.82 (m, 4H), 2.81-2.65 (m, 1H), 2.63-2.54 (m, 1H), 2.10-1.87 (m, 5H), 1.83-1.52 (m, 10H), 1.39 (br s, 2H).

### Example 72. Synthesis of N(1-(2-(4-(5-chloro-4-((2,6-dioxopiperidin-3-yl)amino)-2-fluorophenyl)piperazin-1-yl)ethyl)piperidin-4-yl)-4-((7,7-difluoro-9-isopropyl-5-methyl-6-oxo-6,7,8,9-tetrahydro-5H-pyrimido[4,5-b][1,4]diazepin-2-yl)amino)-2-fluoro-5-methoxybenzamide (Compound 72)

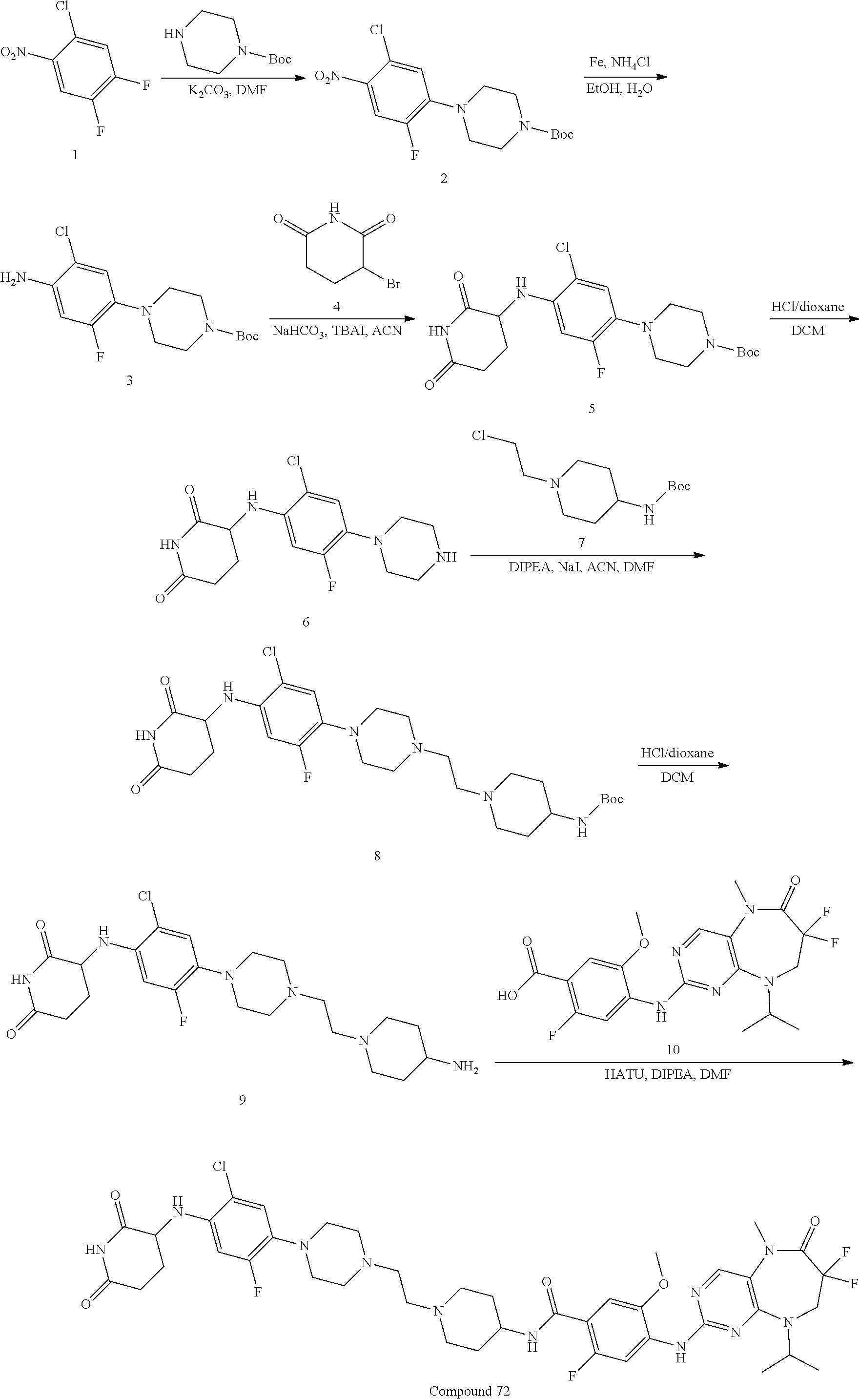

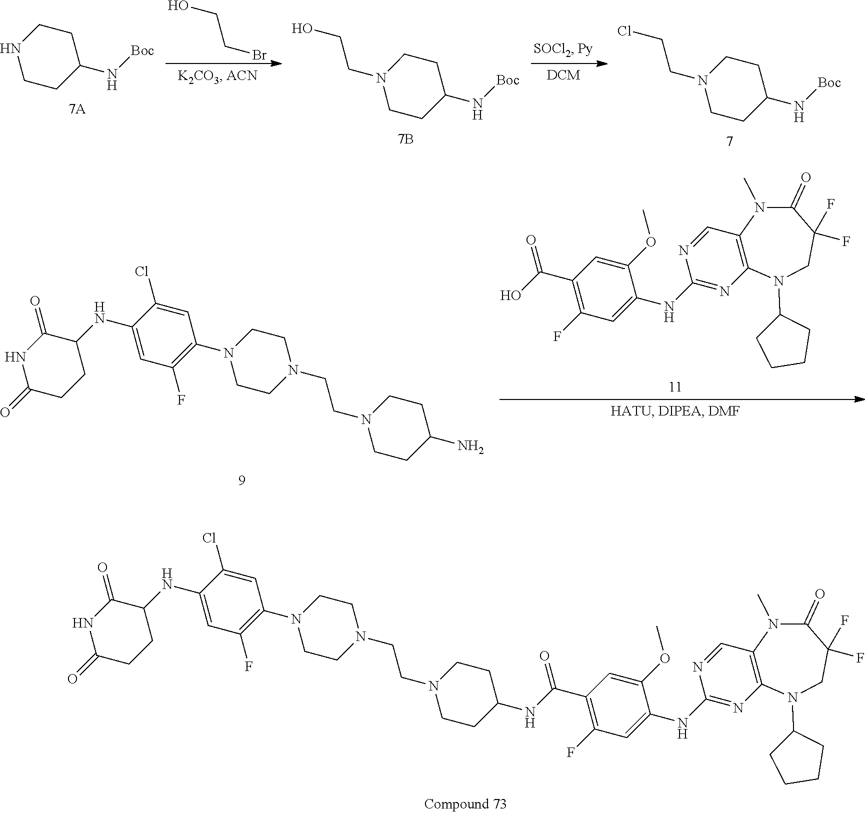

### Step 1. Synthesis of tert-butyl 4-(5-chloro-2-fluoro-4-nitrophenyl)piperazine-1-carboxylate (2)

It was synthesized by similar method described in step 4 of Example 30 to obtain tert-butyl 4-(5-chloro-2-fluoro-4-nitrophenyl)piperazine-1-carboxylate (19 g, crude) as a yellow solid. MS(M-100+H)⁺=260.2

### Step 2. Synthesis of tert-butyl 4-(4-amino-5-chloro-2-fluorophenyl)piperazine-1-carboxylate (3)

It was synthesized by similar method described in step 4 of Example 22 to obtain tert-butyl 4-(4-amino-5-chloro-2-fluorophenyl)piperazine-1-carboxylate (16.66 g, crude) as a yellow oil. MS(M+H)⁺=330.2

### Step 3. Synthesis of tert-butyl 4-(5-chloro-4-((2,6-dioxopiperidin-3-yl)amino)-2-fluorophenyl)piperazine-1-carboxylate (5)

It was synthesized by similar method described in step 3 of Example 51 to obtain tert-butyl 4-(5-chloro-4-((2,6-dioxopiperidin-3-yl)amino)-2-fluorophenyl)piperazine-1-carboxylate (15 g, 30.62 mmol, 60.61% yield, 90% purity) as a yellow solid. MS(M+H)⁺=441.2

### Step 4. Synthesis of 3-((2-chloro-5-fluoro-4-(piperazin-1-yl)phenyl)amino)piperidine-2,6-dione (6)

It was synthesized by similar method described in step 2 of Example 48 to obtain 3-((2-chloro-5-fluoro-4-(piperazin-1-yl)phenyl)amino)piperidine-2,6-dione (11.6 g, crude, HCl) as a blue solid. MS(M+H)⁺=341.3

### Step 5. Synthesis of tert-butyl (1-(2-hydroxyethyl)piperidin-4-yl)carbamate (7B)

It was synthesized by similar method described in step 2 of Example 7 to obtain tert-butyl (1-(2-hydroxyethyl)piperidin-4-yl)carbamate (25 g, 102.32 mmol, 81.97% yield) as a white solid. MS(M+H)⁺=245.3

### Step 6. Synthesis of tert-butyl (1-(2-chloroethyl)piperidin-4-yl)carbamate (7)

To a solution of tert-butyl (1-(2-hydroxyethyl)piperidin-4-yl)carbamate (3 g, 12.28 mmol) in DCM (30 mL) was added Py (1.94 g, 24.56 mmol, 1.98 mL) followed by SOCl₂ (2.19 g, 18.42 mmol, 1.34 mL) at 0° C. The mixture was stirred at 20° C. for 2 hr. TLC (DCM/MeOH=10/1) showed the starting material was consumed and new spots were formed. The reaction mixture was diluted with DCM (150 mL) and H₂O (150 mL), then adjust pH-8 with saturated NaHCO₃ at 0° C., the DCM layer was separated, dried over Na₂SO₄, filtered. The filtrate was concentrated in vacuo. The residue was diluted with hexane (100 mL), then concentrated in vacuo at 40~50° C. to remove pyridine to afford tert-butyl (1-(2-chloroethyl)piperidin-4-yl)carbamate (3.3 g, crude) as a yellow solid. MS(M+H)⁺=263.8

### Step 7. Synthesis of tert-butyl (1-(2-(4-(5-chloro-4-((2,6-dioxopiperidin-3-yl)amino)-2-fluorophenyl)piperazin-1-yl)ethyl)piperidin-4-yl)carbamate (8)

It was synthesized by similar method described in step 1 of Example 48 to obtain tert-butyl (1-(2-(4-(5-chloro-4-((2,6-dioxopiperidin-3-yl)amino)-2-fluorophenyl)piperazin-1-yl)ethyl)piperidin-4-yl)carbamate (2.55 g, 4.50 mmol, 33.93% yield) as a black blue solid. MS(M+H)⁺=567.3

### Step 8. Synthesis of 3-((4-(4-(2-(4-aminopiperidin-1-yl)ethyl)piperazin-1-yl)-2-chloro-5-fluorophenyl)amino)piperidine-2,6-dione (9)

It was synthesized by similar method described in step 4 to obtain 3-((4-(4-(2-(4-aminopiperidin-1-yl)ethyl)piperazin-1-yl)-2-chloro-5-fluorophenyl)amin o)piperidine-2,6-dione (3.4 g, crude, HCl) as a gray solid. MS(M+H)⁺=467.3

### Step 9. Synthesis of N(1-(2-(4-(5-chloro-4-((2,6-dioxopiperidin-3-yl)amino)-2-fluorophenyl)piperazin-1-yl)ethyl)piperidin-4-yl)-4-((7,7-difluoro-9-isopropyl-5-methyl-6-oxo-6,7,8,9-tetrahydro-5H-pyrimido[4,5-b][1,4]diazepin-2-yl)amino)-2-fluoro-5-methoxybenzamide (Compound 72)

It was synthesized by similar method described in step 10 of Example 2 to obtain N(1-(2-(4-(5-chloro-4-((2,6-dioxopiperidin-3-yl)amino)-2-fluorophenyl)piperazin-1-yl)ethyl)piperidin-4-yl)-4-((7,7-difluoro-9-isopropyl-5-methyl-6-oxo-6,7,8,9-tetrahydro-5 H-pyrimido[4,5-b][1,4]diazepin-2-yl)amino)-2-fluoro-5-methoxybenzamide (1.33 g, 1.47 mmol, 49.59% yield, 98% purity) as a yellow solid. MS(M+H)⁺=888.4

¹H NMR (400 MHz, DMSO-d₆) δ=10.87 (s, 1H), 8.32-8.24 (m, 2H), 8.15-7.80 (m, 2H), 7.19 (d, J=6.6 Hz, 1H), 7.02-6.95 (m, 1H), 6.77 (d, J=14.8 Hz, 1H), 5.43-5.37 (m, 1H), 4.95-4.85 (m, 1H), 4.45-4.36 (m, 1H), 4.06 (t, J=13.4 Hz, 2H), 3.92 (s, 3H), 3.41-3.36 (m, 3H), 3.33 (s, 3H), 2.96-2.85 (m, 4H), 2.82-2.71 (m, 1H), 2.64-2.53 (m, 11H), 2.11-1.99 (m, 3H), 1.88-1.71 (m, 2H), 1.67-1.47 (m, 1H), 1.26 (d, J=6.7 Hz, 6H).

### Example 73. Synthesis of N(1-(2-(4-(5-chloro-4-((2,6-dioxopiperidin-3-yl)amino)-2-fluorophenyl)piperazin-1-yl)ethyl)piperidin-4-yl)-4-((9-cyclopentyl-7,7-difluoro-5-methyl-6-oxo-6,7,8,9-tetra hydro-5H-pyrimido[4,5-b][1,4]diazepin-2-yl)amino)-2-fluoro-5-methoxybenzamide (Compound 73)

It was synthesized by similar method described in step 10 of Example 2 to obtain N(1-(2-(4-(5-chloro-4-((2,6-dioxopiperidin-3-yl)amino)-2-fluorophenyl)piperazin-1-yl)ethyl)piperidin-4-yl)-4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamide (1.53 g, 1.64 mmol, 54.51% yield, 98% purity) as a yellow solid. MS(M+H)⁺=914.5

¹H NMR (400 MHz, DMSO-d₆) δ=10.87 (s, 1H), 8.35-8.21 (m, 2H), 8.13-7.92 (m, 2H), 7.19 (d, J=6.8 Hz, 1H), 6.99 (d, J=8.8 Hz, 1H), 6.77 (d, J=14.8 Hz, 1H), 5.44-5.37 (m, 1H), 4.88-4.77 (m, 1H), 4.46-4.36 (m, 1H), 4.08 (t, J=13.8 Hz, 2H), 3.92 (s, 3H), 3.55-3.48 (m, 4H), 3.33 (s, 3H), 2.95-2.87 (m, 4H), 2.83-2.73 (m, 1H), 2.69-2.51 (m, 11H), 2.12-1.88 (m, 6H), 1.80-1.55 (m, 7H).

### Example 74. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methylphenyl)piperazin-1-yl)ethyl)piperidin-1-yl)-3-methoxybenzamide (Compound 74)

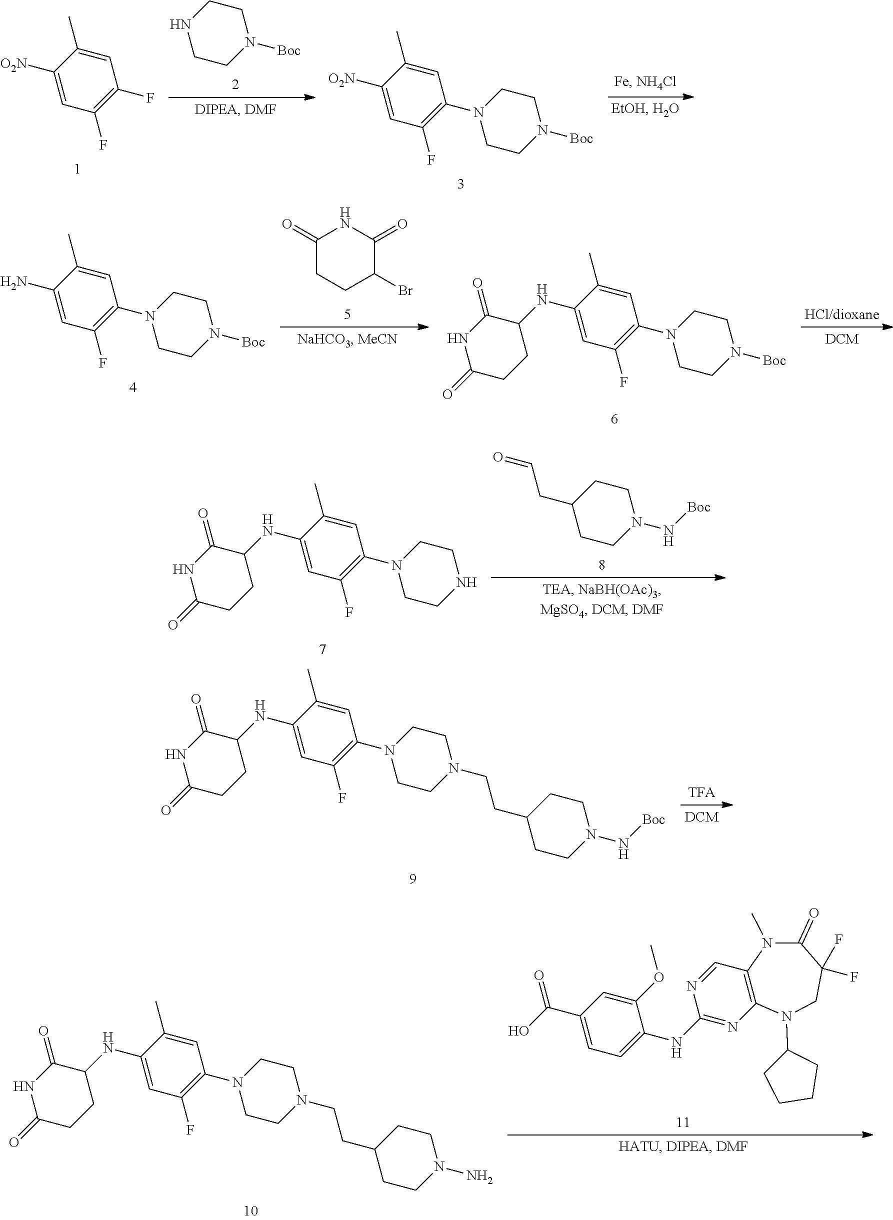

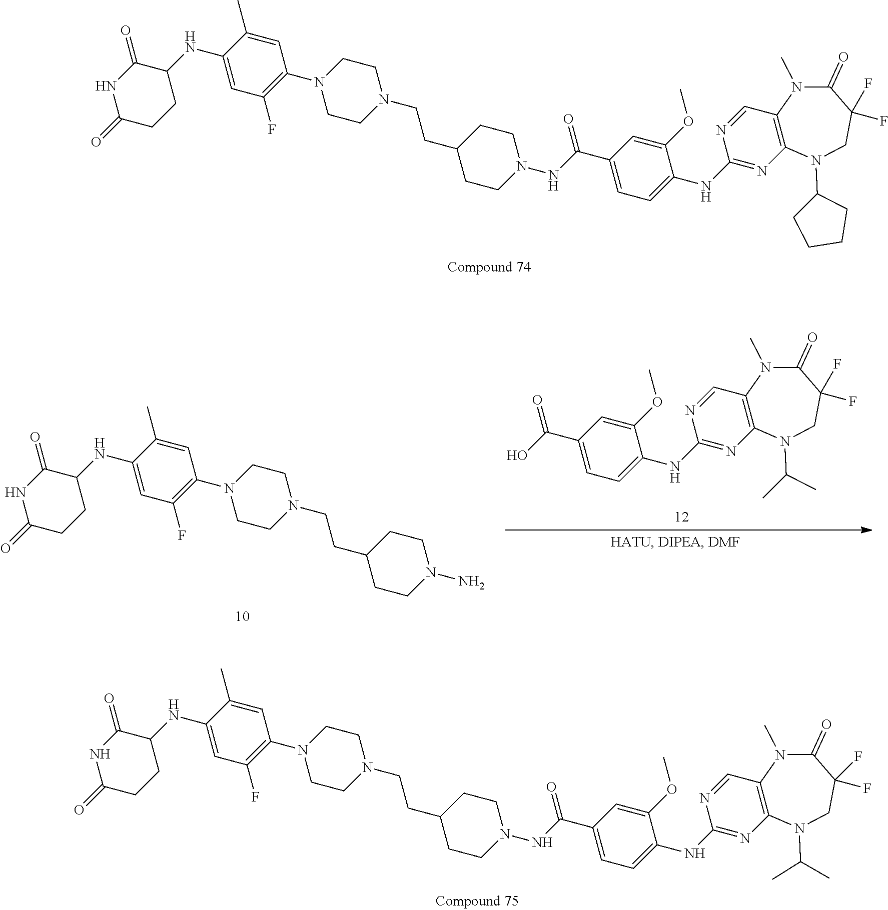 [SMILES: COc1cc(C(=O)NN2CCC(CCN3CCN(c4cc(C)c(NC5CCC(=O)NC5=O)cc4F)CC3)CC2)ccc1Nc1ncc2c(n1)N(C1CCCC1)CC(F)(F)C(=O)N2C ; Cc1cc(N2CCN(CCC3CCN(N)CC3)CC2)c(F)cc1NC1CCC(=O)NC1=O ; COc1cc(C(=O)O)ccc1Nc1ncc2c(n1)N(C(C)C)CC(F)(F)C(=O)N2C ; COc1cc(C(=O)NN2CCC(CCN3CCN(c4cc(C)c(NC5CCC(=O)NC5=O)cc4F)CC3)CC2)ccc1Nc1ncc2c(n1)N(C(C)C)CC(F)(F)C(=O)N2C]

### Step 1. Synthesis of tert-butyl 4-(2-fluoro-5-methyl-4-nitrophenyl)piperazine-1-carboxylate (3)

To a solution of 1,2-difluoro-4-methyl-5-nitrobenzene (2 g, 11.55 mmol) and tert-butyl piperazine-1-carboxylate (2.15 g, 11.55 mmol) in DMF (40 mL) was added DIPEA (4.48 g, 34.66 mmol, 6.04 mL) and the mixture was stirred at 80° C. for 3 h. LCMS showed a peak (86%) with desired mass. The mixture was diluted with water (60 mL), then extracted with EtOAc (30 mL×2). The combined organic layer was washed with brine (20 mL×2), dried over Na₂SO₄ and filtered. The filtrate was concentrated under reduced pressure. The residue was purified by flash silica gel chromatography (20 g SepaFlash® Silica Flash Column, Eluent of 20-50% EtOAc/Petroleum ether gradient @ 200 mL/min) to afford tert-butyl 4-(2-fluoro-5-methyl-4-nitrophenyl)piperazine-1-carboxylate (3.36 g, 9.31 mmol, 80.56% yield, 94% purity) as a yellow solid. MS(M-56+H)⁺=284.2

### Step 2. Synthesis of tert-butyl 4-(4-amino-2-fluoro-5-methylphenyl)piperazine-1-carboxylate (4)

It was synthesized by similar method described in step 4 of Example 22 to obtain tert-butyl 4-(4-amino-2-fluoro-5-methylphenyl)piperazine-1-carboxylate (960 mg, crude) as a yellow oil. MS(M+H)⁺=310.1

### Step 3. Synthesis of tert-butyl 4-(4-((2,6-dioxopiperidin-3-yl)amino)-2-fluoro-5-methylphenyl)piperazine-1-carboxylate (6)

It was synthesized by similar method described in step 5 of Example 22 to obtain tert-butyl 4-(4-((2,6-dioxopiperidin-3-yl)amino)-2-fluoro-5-methylphenyl)piperazine-1-carboxylate (910 mg, 2.16 mmol, 77.86% yield) as a blue solid. MS(M+H)⁺=421.3

### Step 4. Synthesis of 3-((5-fluoro-2-methyl-4-(piperazin-1-yl)phenyl)amino)piperidine-2,6-dione (7)

It was synthesized by similar method described in step 2 of Example 48 to obtain 3-((5-fluoro-2-methyl-4-(piperazin-1-yl)phenyl)amino)piperidine-2,6-dione (860 mg, crude, 2HCl) as a blue solid. MS(M+H)⁺=321.2

### Step 5. Synthesis of tert-butyl (4-(2-(4-(4-((2,6-dioxopiperidin-3-yl)amino)-2-fluoro-5-methylphenyl)piperazin-1-yl)ethyl)piperidin-1-yl)carbamate (9)

It was synthesized by similar method described in step 5 of Example 56 to obtain tert-butyl (4-(2-(4-(4-((2,6-dioxopiperidin-3-yl)amino)-2-fluoro-5-methylphenyl)piperazin-1-yl)ethyl)piperidin-1-yl)carbamate (340 mg, 572.19 mol, 46.88% yield, 92% purity) as a yellow solid. MS(M+H)⁺=547.4

### Step 6. Synthesis of 3-((4-(4-(2-(1-aminopiperidin-4-yl)ethyl)piperazin-1-yl)-5-fluoro-2-methylphenyl) amino)piperidine-2,6-dione (10)

It was synthesized by similar method described in step 6 of Example 3 to obtain 3-((4-(4-(2-(1-aminopiperidin-4-yl)ethyl)piperazin-1-yl)-5-fluoro-2-methylphenyl)amino)piperidine-2,6-dione (350 mg, crude, TFA salt) as a yellow oil. MS(M+H)⁺=447.4

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methylphenyl)piperazin-1-yl)ethyl)piperidin-1-yl)-3-methoxybenzamide (Compound 74)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-m ethylphenyl)piperazin-1-yl)ethyl)piperidin-1-yl)-3-methoxybenzamide (77.9 mg, 62.04 mol, 10.28% yield, 97% purity, 3TFA) as a yellow solid. MS(M+H)⁺=876.4

¹H NMR (400 MHz, DMSO-d₆) δ=10.83 (s, 1H), 9.75-9.32 (m, 2H), 8.29-8.11 (m, 3H), 7.47-7.40 (m, 2H), 6.85-6.78 (m, 1H), 6.55 (d, J=15.4 Hz, 1H), 4.83-4.74 (m, 1H), 4.35 (br dd, J=5.7, 11.7 Hz, 1H), 4.12-4.03 (m, 2H), 3.93 (s, 3H), 3.59-3.53 (m, 2H), 3.33 (s, 3H), 3.31-3.26 (m, 1H), 3.23-3.16 (m, 3H), 3.14-3.08 (m, 2H), 3.01-2.93 (m, 2H), 2.88-2.80 (m, 2H), 2.79-2.72 (m, 1H), 2.08 (s, 3H), 2.06-1.96 (m, 2H), 1.96-1.88 (m, 2H), 1.80-1.53 (m, 13H), 1.42-1.29 (m, 3H).

### Example 75. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methylphenyl)piperazin-1-yl)ethyl)piperidin-1-yl)-3-methoxybenzamide (Compound 75)

It was synthesized by similar method described in step 10 of Example 2 to obtain 4-((7,7-difluoro-9-isopropyl-5-methyl-6-oxo-6,7,8,9-tetrahydro-5H-pyrimido[4,5-b][1,4]diazepin-2-yl)amino)-N-(4-(2-(4-(4-((2,6-dioxopiperidin-3-yl)amino)-2-fluoro-5-methylphenyl)piperazin-1-yl)ethyl)piperidin-1-yl)-3-methoxybenzamide (43.6 mg, 39.76 mol, 11.97% yield, 98.3% purity, 2TFA) as a white solid. MS(M+H)⁺=850.5

¹H NMR (400 MHz, DMSO-d₆) δ=10.82 (s, 1H), 9.64 (br s, 1H), 9.55-9.43 (m, 1H), 8.30-8.16 (m, 3H), 7.49-7.42 (m, 2H), 6.81 (d, J=9.7 Hz, 1H), 6.55 (d, J=15.3 Hz, 1H), 4.94-4.84 (m, 1H), 4.35 (dd, J=5.1, 11.7 Hz, 1H), 4.08 (t, J=13.3 Hz, 2H), 3.93 (s, 3H), 3.35-3.26 (m, 7H), 3.23-3.08 (m, 6H), 3.02-2.92 (m, 2H), 2.88-2.73 (m, 3H), 2.61-2.53 (m, 1H), 2.12-1.95 (m, 5H), 1.82-1.70 (m, 2H), 1.69-1.57 (m, 2H), 1.44-1.32 (m, 3H), 1.24 (d, J=6.7 Hz, 6H).

### Example 76. Synthesis of N(4-(2-(4-(5-cyano-4-((2,6-dioxopiperidin-3-yl)amino)-2-fluorophenyl)piperazin-1-yl)ethyl)piperidin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (Compound 76)

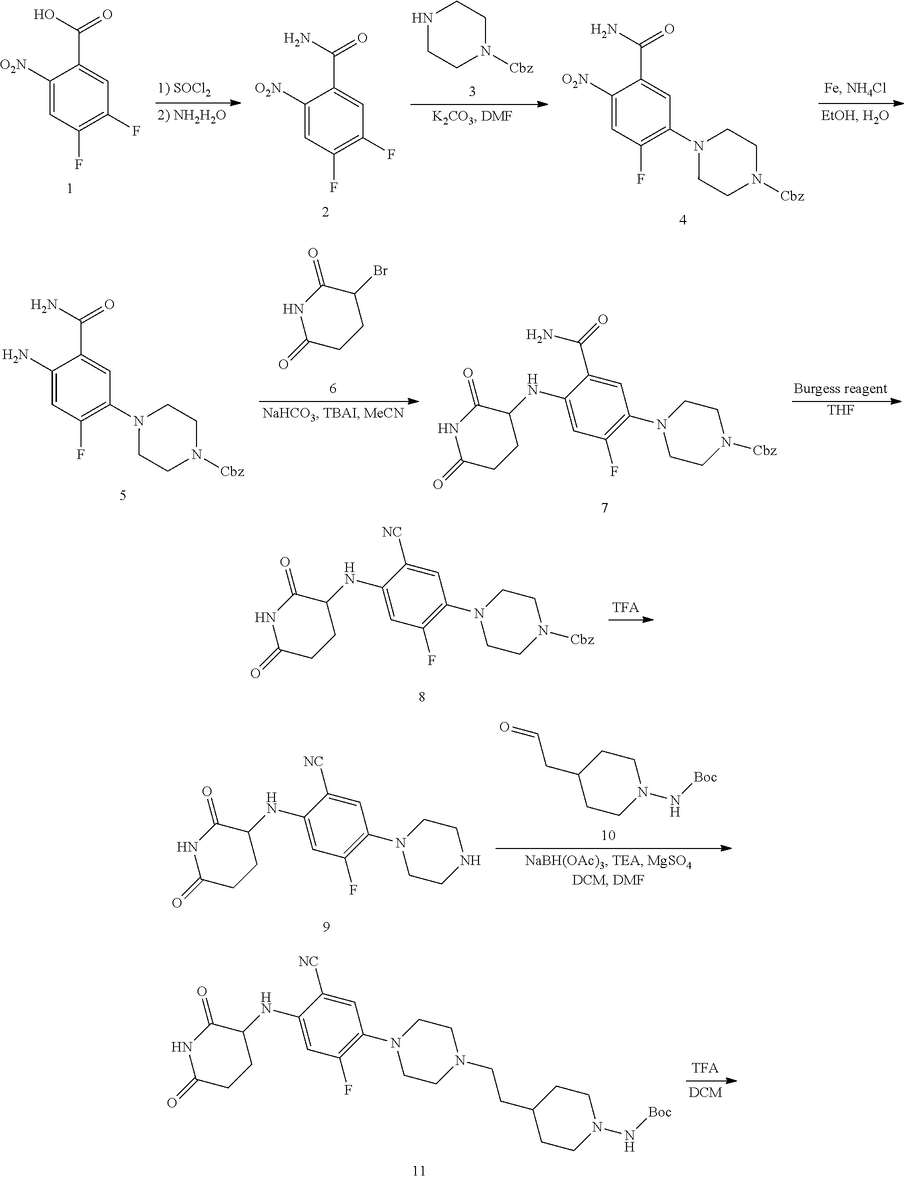

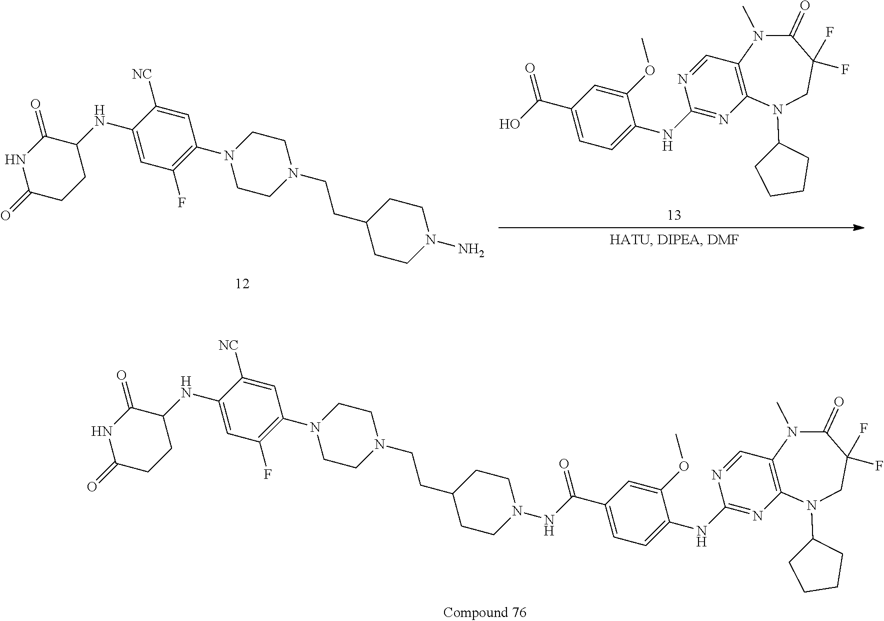 [SMILES: N#Cc1cc(N2CCN(CCC3CCN(N)CC3)CC2)c(F)cc1NC1CCC(=O)NC1=O ; COc1cc(C(=O)O)ccc1Nc1ncc2c(n1)N(C1CCCC1)CC(F)(F)C(=O)N2C ; COc1cc(C(=O)NN2CCC(CCN3CCN(c4cc(C#N)c(NC5CCC(=O)NC5=O)cc4F)CC3)CC2)ccc1Nc1ncc2c(n1)N(C1CCCC1)CC(F)(F)C(=O)N2C]

### Step 1. Synthesis of 4,5-difluoro-2-nitrobenzamide (2)

To a solution of SOCl₂ (65.52 g, 550.73 mmol, 40.00 mL) was slowly added 4,5-difluoro-2-nitrobenzoic acid (10 g, 49.24 mmol) and the mixture was stirred at 70° C. for 14 h. TLC (Petroleum ether:EtOAc=10:1, quenched with MeOH) showed the starting material was consumed and the new spot formed. The mixture was concentrated under reduced pressure (50° C.) to afford 4,5-difluoro-2-nitrobenzoyl chloride (12 g, crude). To a solution of NH₃—H₂O (36.40 g, 311.59 mmol, 40 mL, 30% purity) was slowly added 4,5-difluoro-2-nitrobenzoyl chloride (12 g, crude) at 0° C. and the mixture was stirred at 20° C. for 1 h. A large quantity of white precipitate was formed. The mixture was diluted with water (100 mL) and extracted with EtOAc (50 mL×3), the combined organic layer was washed with water (50 mL×2), dried over Na₂SO₄ and filtered. The filtrate was concentrated under reduced pressure to afford 4,5-difluoro-2-nitrobenzamide (8.6 g, crude) as a white solid.

¹H NMR (400 MHz, DMSO-d₆) δ=8.35-8.29 (m, 1H), 8.17 (s, 1H), 7.88-7.80 (m, 2H)

### Step 2. Synthesis of benzyl 4-(5-carbamoyl-2-fluoro-4-nitrophenyl)piperazine-1-carboxylate (4)

To a solution of 4,5-difluoro-2-nitrobenzamide (4.3 g, 21.28 mmol) and benzyl piperazine-1-carboxylate (5.6 g, 25.42 mmol, 4.90 mL) in DMF (100 mL) was added K₂CO₃ (8.82 g, 63.83 mmol) and the mixture was stirred at 25° C. for 14 h. LCMS showed 94% of the desired mass. The mixture was combined with other batch (0.5 g scales) and then diluted with water (100 mL). The mixture was filtered and the filter cake was washed with MTBE (100 mL) and water (50 mL). The filter cake was collected and the solvent was removed under reduced pressure to afford benzyl 4-(5-carbamoyl-2-fluoro-4-nitrophenyl)piperazine-1-carboxylate (9.7 g, crude) as a yellow solid.

¹H NMR (400 MHz, DMSO-d₆) δ=8.02-7.90 (m, 2H), 7.64 (s, 1H), 7.41-7.36 (m, 4H), 7.36-7.30 (m, 1H), 7.06 (d, J=8.6 Hz, 1H), 5.12 (s, 2H), 3.62-3.53 (m, 4H), 3.31-3.26 (m, 4H)

### Step 3. Synthesis of benzyl 4-(4-amino-5-carbamoyl-2-fluorophenyl)piperazine-1-carboxylate (5)

It was synthesized by similar method described in step 4 of Example 22 to obtain benzyl 4-(4-amino-5-carbamoyl-2-fluorophenyl)piperazine-1-carboxylate (3.52 g, crude) as a yellow solid. MS(M+H)⁺=373.3

### Step 4. Synthesis of benzyl 4-(5-carbamoyl-4-((2,6-dioxopiperidin-3-yl)amino)-2-fluorophenyl)piperazine-1-carboxylate (7)

It was synthesized by similar method described in step 3 of Example 51 to obtain benzyl 4-(5-carbamoyl-4-((2,6-dioxopiperidin-3-yl)amino)-2-fluorophenyl)piperazine-1-carboxylate (1.88 g, 3.77 mmol, 39.90% yield, 97% purity) as a yellow solid. MS(M+H)⁺=484.3.

### Step 5. Synthesis of benzyl 4-(5-cyano-4-((2,6-dioxopiperidin-3-yl)amino)-2-fluorophenyl)piperazine-1-carboxylate (8)

To a solution of benzyl 4-(5-carbamoyl-4-((2,6-dioxopiperidin-3-yl)amino)-2-fluorophenyl)piperazine-1-carboxylate (1.44 g, 2.98 mmol) in THF (60 mL) was added Burgess reagent (710 mg, 2.98 mmol) at 0° C. and the mixture was stirred at 0° C. for 30 min. LCMS showed 55% of the starting material remained and 41% of the desired mass. Burgess reagent (568 mg, 2.38 mmol) was added and the mixture was stirred at 0° C. for 30 min. LCMS showed 74% of the desired mass. The mixture was quenched with saturated aq. NaHCO₃ (20 mL) and then the mixture (pH=8) was extracted with DCM (50 mL×3), the combined organic layer was washed with water (20 mL), dried over Na₂SO₄ and filtered. The filtrate was concentrated under reduced pressure. The residue was purified by flash silica gel chromatography (12 g SepaFlash® Silica Flash Column, Eluent of 50-100% EtOAc/Petroleum ether gradient @ 100 mL/min) to afford benzyl 4-(5-cyano-4-((2,6-dioxopiperidin-3-yl)amino)-2-fluorophenyl)piperazine-1-carboxylate (1.1 g, 2.36 mmol, 79.35% yield) as a yellow solid. MS(M+H)⁺=466.3.

### Step 6. Synthesis of 2-((2,6-dioxopiperidin-3-yl)amino)-4-fluoro-5-(piperazin-1-yl)benzonitrile (9)

It was synthesized by similar method described in step 2 of Example 4 to obtain 2-((2,6-dioxopiperidin-3-yl)amino)-4-fluoro-5-(piperazin-1-yl)benzonitrile (730 mg, 1.46 mmol, 61.73% yield, 89% purity, TFA) as a white solid. MS(M+H)⁺=332.1

### Step 7. Synthesis of tert-butyl (4-(2-(4-(5-cyano-4-((2,6-dioxopiperidin-3-yl)amino)-2-fluorophenyl)piperazin-1-yl)ethyl)piperidin-1-yl)carbamate (11)

It was synthesized by similar method described in step 5 of Example 56 to obtain tert-butyl (4-(2-(4-(5-cyano-4-((2,6-dioxopiperidin-3-yl)amino)-2-fluorophenyl)piperazin-1-yl)ethyl)piperidin-1-yl)carbamate (560 mg, 984.11 mol, 67.46% yield, 98% purity) as a yellow solid. MS(M+H)⁺=558.5

### Step 8. Synthesis of 5-(4-(2-(1-aminopiperidin-4-yl)ethyl)piperazin-1-yl)-2-((2,6-dioxopiperidin-3-yl)amino)-4-fluorobenzonitrile (12)

It was synthesized by similar method described in step 6 of Example 3 to obtain 5-(4-(2-(1-aminopiperidin-4-yl)ethyl)piperazin-1-yl)-2-((2,6-dioxopiperidin-3-yl)amin o)-4-fluorobenzonitrile (580 mg, crude, TFA) as a yellow oil. MS(M+H)⁺=458.3

### Step 9. Synthesis of N(4-(2-(4-(5-cyano-4-((2,6-dioxopiperidin-3-yl)amino)-2-fluorophenyl)piperazin-1-yl)ethyl)piperidin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (Compound 76)

It was synthesized by similar method described in step 10 of Example 2 to obtain N(4-(2-(4-(5-cyano-4-((2,6-dioxopiperidin-3-yl)amino)-2-fluorophenyl)piperazin-1-yl)ethyl)piperidin-1-yl)-4-((9-cyclopentyl-7,7-difluoro-5-methyl-6-oxo-6,7,8,9-tetrahydro-5 H-pyrimido[4,5-b][1,4]diazepin-2-yl)amino)-3-methoxybenzamide (641.17 mg, 701.20 μmol, 69.10% yield, 97% purity) as an off-white solid. MS(M+H)⁺=887.5.

¹H NMR (400 MHz, DMSO-d₆) δ=10.86 (s, 1H), 9.27 (s, 1H), 8.31-8.21 (m, 2H), 7.96 (s, 1H), 7.48-7.38 (m, 2H), 7.18 (d, J=9.3 Hz, 1H), 6.82 (d, J=15.4 Hz, 1H), 6.12 (d, J=7.8 Hz, 1H), 4.84-4.69 (m, 1H), 4.56-4.45 (m, 1H), 4.09-3.99 (m, 2H), 3.92 (s, 3H), 3.34-3.30 (m, 6H), 3.03-2.96 (m, 2H), 2.92-2.88 (m, 4H), 2.80-2.68 (m, 3H), 2.60-2.52 (m, 2H), 2.38-2.30 (m, 2H), 2.15-2.01 (m, 2H), 1.99-1.88 (m, 2H), 1.77-1.66 (m, 4H), 1.65-1.23 (m, 4H), 1.47-1.37 (m, 2H), 1.35-1.23 (m, 3H).

### Example 77. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(6-((2,6-dioxopiperidin-3-yl)amino)pyridin-3-yl)piperazin-1-yl)ethyl)piperidin-1-yl)-3-methoxybenzamide (Compound 77)

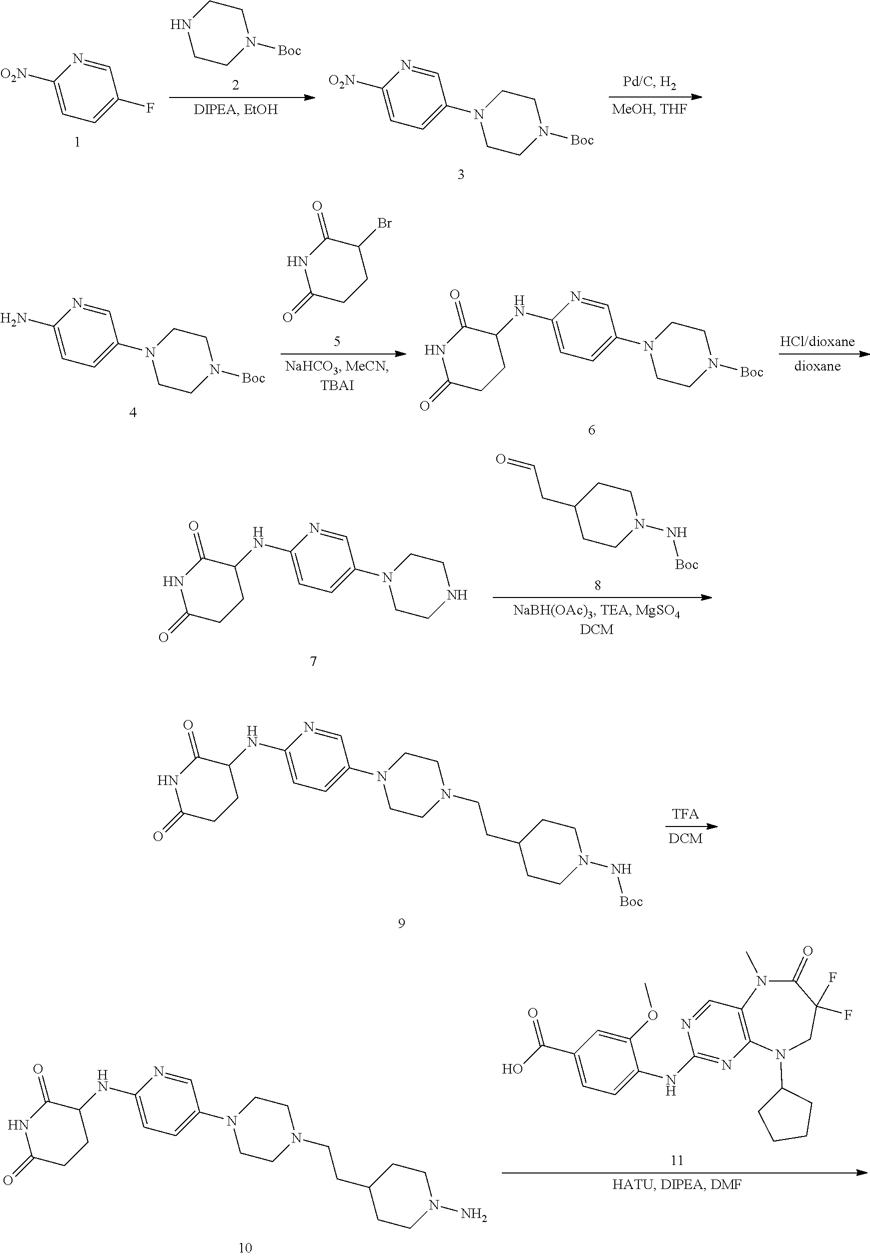

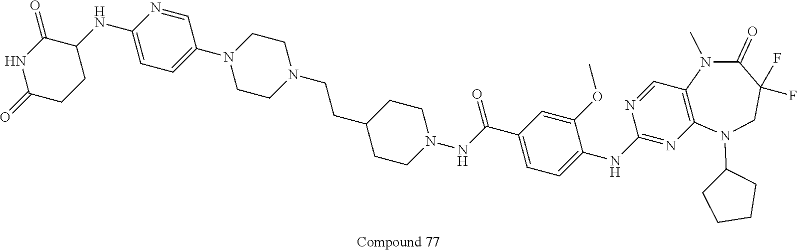 [SMILES: COc1cc(C(=O)NN2CCC(CCN3CCN(c4ccc(NC5CCC(=O)NC5=O)nc4)CC3)CC2)ccc1Nc1ncc2c(n1)N(C1CCCC1)CC(F)(F)C(=O)N2C]

### Step 1. Synthesis of tert-butyl 4-(6-nitro-pyrid-3-yl)piperazine-1-carboxylate (3)

To a solution of 5-fluoro-2-nitro-pyridine (15.00 g, 105.57 mmol) in EtOH (200 mL) were added tert-butyl piperazine-1-carboxylate (23.59 g, 126.68 mmol) and DIPEA (27.29 g, 211.14 mmol, 36.78 mL). The mixture was stirred at 80° C. for 16 hours. LCMS showed a main peak (~99%) with desired mass. The reaction mixture was concentrated under reduced pressure to remove solvent. The residue was diluted with water (300 mL), then extracted with EtOAc (300 mL×3). The combined organic layers were washed with brine (100 mL×3), dried over Na₂SO₄, filtered and concentrated under reduced pressure to afford tert-butyl 4-(6-nitro-3-pyridyl)piperazine-1-carboxylate (31.00 g, 98.53 mmol, 93.33% yield). MS(M+H)⁺=309.2.

¹H NMR (400 MHz, DMSO-d₆) δ=8.23 (d, J=2.8 Hz, 1H), 8.16 (d, J=9.2 Hz, 1H), 7.46 (dd, J=9.2, 2.8 Hz, 1H), 3.54-3.50 (m, 4H), 3.50-3.46 (m, 4H), 1.42 (s, 9H).

### Step 2. Synthesis of tert-butyl 4-(6-amino-pyrid-3-yl)piperazine-1-carboxylate (4)

To a solution of tert-butyl 4-(6-nitro-3-pyridyl)piperazine-1-carboxylate (17.00 g, 54.03 mmol) in MeOH (300 mL) and THF (300 mL) was added wet Pd/C (1.70 g, 10% purity) under N₂ atmosphere. The suspension was degassed under vacuum and purged with H₂ several times. The mixture was stirred at 25° C. for 16 hours under H₂ (15 psi) atmosphere. TLC indicated one major new spot with larger polarity was formed. The reaction mixture was filtrated and the filtrate was concentrated to afford tert-butyl 4-(6-amino-3-pyridyl)piperazine-1-carboxylate (15.00 g, 53.89 mmol, 99.73% yield) as a brown solid. MS(M+H)⁺=279.2.

### Step 3. Synthesis of tert-butyl 4-(6-((2,6-dioxo-piperid-3-yl)amino)-3-pyridyl)piperazine-1-carboxylate (6)

It was synthesized by similar method described in step 3 of Example 51 to obtain tert-butyl 4-(6-((2,6-dioxo-3-piperidyl)amino)-3-pyridyl)piperazine-1-carboxylate (6.20 g, 15.76 mmol, 29.25% yield) as a pale yellow solid. MS(M+H)⁺=390.2.

¹H NMR (400 MHz, CDCl₃) δ=7.97 (s, 1H), 7.79 (d, J=2.8 Hz, 1H), 7.21 (dd, J=8.8, 2.8 Hz, 1H), 6.55 (d, J=8.8 Hz, 1H), 5.10 (d, J=4.0 Hz, 1H), 4.69-4.59 (m, 1H), 3.64-3.53 (m, 4H), 3.01-2.93 (m, 4H), 2.85-2.80 (m, 2H), 2.78-2.70 (m, 1H), 1.96-1.81 (m, 1H), 1.42 (s, 9H).

### Step 4. Synthesis of 3-((5-piperazin-1-yl-pyrid-2-yl)amino)piperidine-2,6-dione (7)

It was synthesized by similar method described in step 5 of Example i to obtain 3-((5-piperazin-1-yl-2-pyridyl)amino)piperidine-2,6-dione (3.31 g, crude, HCl salt) as an off-white solid. MS(M+H)⁺=290.1.

### Step 5. Synthesis of tert-butyl (4-(2-(4-(6-((2,6-dioxopiperidin-3-yl)amino)pyridin-3-yl)piperazin-1-yl)ethyl)piperidin-1-yl)carbamate (9)

It was synthesized by similar method described in step 6 of Example 1 to obtain tert-butyl (4-(2-(4-(6-((2,6-dioxopiperidin-3-yl)amino)pyridin-3-yl)piperazin-1-yl)ethyl)piperidin-1-yl)carbamate (3.30 g, 6.14 mmol, 75.53% yield) as a pale yellow solid. MS(M+H)⁺=516.4.

### Step 6. Synthesis of 3-((5-(4-(2-(1-aminopiperidin-4-yl)ethyl)piperazin-1-yl)pyridin-2-yl)amino)piperidine-2,6-dione (10)

It was synthesized by similar method described in step 6 of Example 3 to obtain 3-((5-(4-(2-(1-aminopiperidin-4-yl)ethyl)piperazin-1-yl)pyridin-2-yl)amino)piperidine-2,6-dione (3.25 g, crude, TFA) as a pale yellow oil. MS(M+H)⁺=416.3.

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(6-((2,6-dioxopiperidin-3-yl)amino)pyridin-3-yl)piperazin-1-yl)ethyl)piperidin-1-yl)-3-methoxybenzamide (Compound 77)

It was synthesized by similar method described in step 10 of Example 2 to obtain 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(6-((2,6-dioxopiperidin-3-yl)amino)pyridin-3-yl) piperazin-1-yl)ethyl)piperidin-1-yl)-3-methoxybenzamide (2.05 g, 2.36 mmol, 38.52% yield, 97.4% purity) as a white solid. MS(M+H)⁺=845.4.

¹H NMR (400 MHz, DMSO-d₆) δ=10.74 (s, 1H), 9.27 (s, 1H), 8.33-8.16 (m, 2H), 7.96 (s, 1H), 7.68-7.58 (m, 1H), 7.48-7.37 (m, 2H), 7.22 (dd, J=9.2, 2.8 Hz, 1H), 6.55 (d, J=8.8 Hz, 1H), 6.40 (d, J=7.6 Hz, 1H), 4.83-4.71 (m, 1H), 4.69-4.61 (m, 1H), 4.04 (t, J=14.0 Hz, 2H), 3.93 (s, 3H), 3.37-3.32 (m, 3H), 3.32-3.30 (m, 3H), 3.04-2.97 (m, 2H), 2.97-2.86 (m, 4H), 2.83-2.69 (m, 3H), 2.57-2.51 (m, 2H), 2.39-2.30 (m, 2H), 2.13-2.04 (m, 1H), 2.04-1.87 (m, 3H), 1.77-1.66 (m, 4H), 1.66-1.54 (m, 4H), 1.47-1.38 (m, 2H), 1.36-1.22 (m, 3H).

### EXPERIMENTAL EXAMPLES

### 1. Western Blot Assay for PLK1

### (1) Culture of HeLa Cell Line

The HeLa cell line was purchased from Korea Cell Line Bank (KCLB), Seoul, Korea. The passage in cell culture was maintained at P115 to P125.

For cell counting, cell counter (Thermo Fisher Scientific Inc., Cat. No. AMQAX1000) and 0.4% trypan blue solution (DYNEBIO, Cat. No. CBT3710; Lot. No. 20231017) were used.

For cell culture, DMEM (Gibco, Cat. No. 11995-65; Lot. No. 2740317), FBS (Gibco, Cat. No. 16000-044; Lot. No. 2716756P), Penicillin/Streptomycin (PS) (Gibco, Cat. No. 15140-122; Lot. No. 2585636), 100 mm² cell culture dish (SPL, Cat. No. 20100), 150 mm² cell culture dish (SPL, Cat. No. 20150), 12-well culture plate (SPL, Cat. No. 30012), PBS pH 7.4 (Gibco, Cat. No. 10010-023; Lot. No. 2085080), TrypLE™ Express (Gibco, Cat. No. 12605-010; Lot No. 2070638), Counting Chamber (Hematocytometer) (Hirschmann, Cat. No. 8100204), and 0.4% Trypan Blue Solution were used.

### (2) Treatment of Compounds of the Present Invention

2×10⁵ cells were seeded for each well of a 12-well plate (SPL), and the cells were cultured in the culture medium in a total volume of 2 mL.

The compounds of Examples were completely dissolved in DMSO(Sigma-Aldrich Cat. No. D2438, Lot. No. RNBJ9566) and used in the experiment, and thymidine was completely dissolved in DW and used in the experiment. For thymidine block, the products were treated with 2 mM of thymidine (Sigma-Aldrich Cat. No. T9250-5G) and then incubated for 24 hours.

For release and chemical treatment, the medium was suctioned and washed 3 times with 1×PBS. Complete media was added, followed by incubation for 4 hours in a CO₂ incubator (Thermo Fisher Scientific, Cat. No. 4111). Each compound was diluted three folds from the highest concentration of 3 μM to the lowest concentration and then incubated for 6 hours again.

### (3) Western Blotting

For SDS-PAGE and Western blotting, 1×RIPA lysis buffer (Rockland, Cat. No. MB-030-0050; Lot. No. 49402), 100× Protease Inhibitor Cocktail (Quartett, Cat. No. PPI1015; Lot. No. PCO50039472), Pierce™ BCA protein assay kit (ThermoScientific, Cat. No. 23225; Lot. No. XL357438), albumin standard (ThermoScientific, Cat. No. 23209; Lot. No. UI389317), 10× Tris/Glycine/SDS buffer (Biosesang, Cat. No. TR2015-000-00; Lot. No. HT1522124D); 10×TBS with Tween20 (Biosesang, Cat. No. TR2007-100-74; Lot. No. P07122X22C), 4× Laemmli sample buffer (Bio-rad, Cat. No. 1610747; Lot. No. 64548487), P-mercaptoethanol (Bio-rad, Cat. No. 1610710; Lot. No. 64408840), 1 M sodium azide solution (Sigma-Aldrich, Cat. No. 08591-1 mL-F; Lot. No. BCBV4989), a-Rabbit pAb to Ms IgG (abcam, Cat. No. ab97046; Lot. No. GR3252115-1), a-Goat pAb to Rb IgG (CST, Cat. No. 7074S; Lot. No. 28), α-GAPDH (abeam, Cat. No. ab8245; Lot. No. GR3275542-2), α-PLK1 (CST, Cat. No. 208G4), NICSROWEST™ ECL PICO substrate (BIONICS, Cat. No. BNROW-008; Lot. No. W08-23MH1018), Ponceau S solution (Sigma-Aldrich, Cat. No. P7170; Lot. No. SLCL7892), Difco™ Skim milk (BD, Cat. No. 232100; Lot. No. 2339387), and mini-protean TGX precast gel 4-15% (Bio-rad, Cat. No. 4561086; Lot. No. 64567916) were used.

For cell harvesting, the cells were first separated from the plate using trypsin and then washed with the medium and PBS. Specifically, the medium was suctioned off and washed with 1 mL of PBS, and PBS was suctioned off. The cells were treated with 0.5 mL TrypLE™ Express at 37° C. for 7 minutes to separate the cells, and then 0.5 mL of complete medium was added to collect 1 mL of cell culture solution. Then, 1 mL of the cell collection solution was centrifuged at 8,000 rpm for 120 seconds, and the supernatant was removed. After washing with 0.2 mL of PBS, the PBS was removed.

For cell lysis, a lysis buffer was added and cell debris was removed to obtain a cell lysate. Specifically, the cells were treated with 70 μL of 1×RIPA buffer containing a protease inhibitor and incubated for 30 minutes on ice. Then, the cells were centrifuged at 4° C. and 15,000 rpm for 10 minutes to obtain a cell lysate.

Then, a standard curve was obtained using the BCA assay, and the protein mass in the lysate was quantified by substituting the curve equation. The mixture was incubated at 37° C. for 30 minutes using 20 μL of standard or sample solution, and 200 μL of BCA or Bradford solution, and measured at 562 nm absorbance. Samples were prepared by adding 4× sample buffer so that the quantity of protein added to each well was 15 g.

Sodium dodecyl sulfate polyacrylamide gel electrophoresis (SDS-PAGE) was performed by setting a running time of 100 minutes at 120 V on a mini-protean TGX precast gel 4-15%. Transferring was performed on Tetra blotting module of the wet blotting system. After staining using Ponceau S solution, blocking was performed for 1 hour with a blocking buffer (Thermo). After washing with 1×TBS containing 0.05% Tween 20, the product was reacted at 4° C. for 16 hours with anti-PLK1 (CST) antibody (1:500) or anti-GAPDH (abeam) antibody (1:10,000) in 1×TBS-T as a primary antibody. After washing three times for 10 minutes with 1×TBS containing 0.05% Tween20, the product was reacted at room temperature for 1 hour with antimouse antibody (abcam) (1:10000) or anti-rabbit antibody (CST) (1:5000) in 1×TBST as a secondary antibody. Then, after washing three times for 10 minutes with 1×TBS containing 0.05% Tween 20, the product was detected with an ECL working solution (1:1).

To analyze the results, an image analyzer (GE) was used to obtain final blot data. Asa result, it was confirmed that all of the compounds of the present invention degraded PLK1 protein significantly.

### 2. Luciferase Assay for PLK1

### (1) Preparation and Culture of HeLa LgBit (Plk1-HiBit KI) Cell Line

A HeLa cell line in which the LgBit vector was transfected and expressed stably was prepared. Then, after constructing gRNA and donor to express the HiBit amino acid sequence behind the C-terminal of the PLK1 gene, which was inherent in the cell, it was inserted into the cell together with a vector capable of expressing CRISPR/Cas9. Only the cells in which the insertion was completed and knock-in had progressed were selected, sub-cultured and used.

For cell culture, DMEM (Gibco), FBS (Gibco), PS (Gibco), 100 nm² cell culture dish (SPL), 150 nm² cell culture dish (SPL), 96-well culture plate (SPL), PBS pH 7.4 (Gibco), TrypLE™ Express (Gibco), Cell counter (Thermo Fisher Scientific) and 0.4% Trypan Blue Solution (DYNEBIO) were used.

### (2) Treatment of Compounds of the Present Invention and Method of Luciferase Assay

The compounds of Examples were completely dissolved in DMSO (Sigma-Aldrich) and used in the experiment.

In the case of HeLa LgBit (Plk1-HiBit KI), the compounds were treated after being released after thymidine block, and the process was as follows. Thymidine (Sigma-Aldrich) was completely dissolved in ddH₂O and used in the experiment. For thymidine block, the products were treated with 2 mM of thymidine and then incubated for 24 hours. For release and chemical treatment, the medium was suctioned and washed with 1×PBS. TrypLE™ (Gibco) was added and incubated in 37° C. CO₂ incubator (Thermo Fisher Scientific) for 5 mins. Cells neutralized by adding complete media were counted through a counter. For each well of a 96-well culture plate (SPL), 3.3×10⁴ cells and a total medium volume of 150 μl were seeded and incubated in a CO₂ incubator.

Each cell line was incubated in a CO₂ incubator for 18 hours, and Endurazine (Promega, Cat. No. N257B) was added to each well to make up 4% of the total volume. After adding compounds of Examples in a 96-well white plate (SPL) to a concentration of 300 nM, the wavelength of the plate reader (BMG Labtech, CLARIOstar Plus) was set to 470-480 nm, and then the luminescence was tracked in real time. After 9 hours, the luminescence value was obtained and displayed as a bar graph through an Excel program.

The results are shown in Table 2 below.

TABLE 2

| Exemplary | | Exemplary | |
|---|---|---|---|
| Compound No. | Activity | Compound No. | Activity |
| 1 | ++ | 2 | ++ |
| 3 | ++ | 4 | ++ |
| 5 | ++ | 6 | +++ |
| 7 | ++ | 8 | ++ |
| 9 | ++ | 10 | +++ |
| 11 | ++ | 12 | +++ |
| 13 | +++ | 14 | +++ |
| 15 | +++ | 16 | +++ |
| 17 | +++ | 18 | ++ |
| 19 | ++ | 20 | +++ |
| 21 | +++ | 22 | +++ |
| 23 | ++ | 24 | ++ |
| 25 | ++ | 26 | ++ |
| 27 | +++ | 28 | +++ |
| 29 | +++ | 30 | +++ |
| 31 | +++ | 32 | ++ |
| 33 | ++ | 34 | ++ |
| 35 | ++ | 36 | +++ |
| 37 | ++ | 38 | +++ |
| 39 | ++ | 40 | +++ |
| 41 | ++ | 42 | ++ |
| 43 | ++ | 44 | +++ |
| 45 | ++ | 46 | ++ |
| 47 | ++ | 48 | ++ |
| 49 | ++ | 50 | ++ |
| 51 | +++ | 52 | ++ |
| 53 | ++ | 54 | +++ |
| 55 | +++ | 56 | ++ |
| 57 | +++ | 58 | +++ |
| 59 | ++ | 60 | ++ |
| 61 | ++ | 62 | ++ |
| 63 | +++ | 64 | ++ |
| 65 | ++ | 66 | ++ |
| 67 | ++ | 68 | ++ |
| 69 | ++ | 70 | +++ |
| 71 | +++ | 72 | ++ |
| 73 | ++ | 74 | +++ |
| 75 | +++ | 76 | ++ |
| 77 | ++ | | |

In Table 2, Activity represents the ratio of the luminescence value of each Exemplary Compound treatment group to DMSO treatment group (+++: <0.3, ++: <0.5, +: <0.7).

### 3. Cell Viability Assay for NCI-H69, NCI-H526 Cell Lines

### (1) Culture of NCI-H69, NCI-H526 Cell Lines

The NCI-H69 (hereafter H69) and NCI-H526 (hereafter H526) cell lines were purchased from KCLB. For cell culture, RPMI 1640 (Gibco, Cat. No. 22400-089; Lot. No. 2537146), FBS (Gibco), PS (Gibco), 75T cell culture flask (SPL, Cat. No. 71075), 175T cell culture flask (SPL, Cat. No. 71175), 96-well cell culture plate (SPL), PBS pH 7.4 (Gibco), TrypLE™ Express (Gibco), Cell Counter (Thermo Fisher Scientific), and 0.4% Trypan Blue Solution (DYNBIO) were used.

### (2) Treatment of Compounds of the Present Invention and Method of Cell Viability Assay

The compounds of Examples were completely dissolved in DMSO (Sigma Aldrich) and used in the experiment. 3×10⁴ cells were seeded for each well of a 96-well plate (SPL), and the cells were cultured in total volume of 150 g. Each compound was diluted 3-folds from the highest concentration of 3000 nM to the lowest concentration of 0.46 nM. After treating the compound to each well to make the total volume of 200 μl, it was cultured in a CO₂ incubator (Thermo Fisher Scientific) for 5 days. Then, after treating EZ-Cytox (DOGEN, Cat. No. EZ-3000, Lot. No. DLS2308) 20 g in each well, it was cultured in CO₂ incubator for 4 hours. The absorbance of the completely cultured sample was measured by setting the wavelength of a plate reader (BMG Labtech) to 450 nM, and was measured after shaking for 3 minutes in a plate reader before measurement. The final measured value was arranged with Excel program, a graph was displayed through Prism-GraphPad program, and the IC₅₀ value was measured.

The results of cell viability for H526 and H69 cell line are shown in Table 3 and Table 4, respectively.

TABLE 3
Cell Viability Assay for H526 cell line

| Exemplary | | Exemplary | |
|---|---|---|---|
| Compound No. | Activity | Compound No. | Activity |
| 1 | A | 2 | B |
| 3 | B | 4 | A |
| 5 | C | 6 | C |
| 7 | B | 8 | A |
| 9 | A | 10 | A |
| 11 | A | 12 | D |
| 13 | A | 14 | A |
| 15 | A | 16 | A |
| 17 | A | 18 | A |
| 19 | A | 20 | A |
| 21 | A | 22 | B |
| 23 | A | 24 | B |
| 25 | A | 26 | A |
| 27 | A | 28 | A |
| 29 | A | 30 | A |
| 31 | A | 32 | A |
| 33 | A | 34 | A |
| 35 | A | 36 | A |
| 37 | B | 38 | A |
| 39 | A | 40 | A |
| 41 | A | 42 | A |
| 43 | A | 44 | B |
| 45 | D | 46 | B |
| 47 | B | 48 | A |
| 49 | A | 50 | B |
| 51 | A | 52 | A |
| 53 | A | 54 | A |
| 55 | A | 56 | A |
| 57 | A | 58 | A |
| 59 | A | 60 | B |
| 61 | A | 62 | A |
| 63 | A | 64 | A |
| 65 | C | 66 | D |
| 67 | A | 68 | B |
| 69 | A | 70 | A |
| 71 | A | 72 | B |
| 73 | B | 74 | A |
| 75 | A | 76 | A |
| 77 | A | | |

TABLE 4
Cell Viability Assay for H69 cell line

| Exemplary | | Exemplary | |
|---|---|---|---|
| Compound No. | Activity | Compound No. | Activity |
| 1 | B | 2 | C |
| 3 | C | 4 | A |
| 5 | C | 6 | B |
| 7 | B | 8 | A |
| 9 | A | 10 | A |
| 11 | A | 12 | D |
| 13 | A | 14 | A |
| 15 | A | 16 | A |
| 17 | A | 18 | A |
| 19 | A | 20 | A |
| 21 | A | 22 | B |
| 23 | B | 24 | C |
| 25 | C | 26 | A |
| 27 | A | 28 | A |
| 29 | A | 30 | A |
| 31 | A | 32 | A |
| 33 | A | 34 | A |
| 35 | B | 36 | B |
| 37 | C | 38 | A |
| 39 | A | 40 | A |
| 41 | A | 42 | B |
| 43 | A | 44 | C |
| 45 | D | 46 | C |
| 47 | B | 48 | B |
| 49 | B | 50 | C |
| 51 | A | 52 | A |
| 53 | B | 54 | A |
| 55 | A | 56 | A |
| 57 | A | 58 | A |
| 59 | B | 60 | B |
| 61 | A | 62 | A |
| 63 | B | 64 | A |
| 65 | D | 66 | D |
| 67 | A | 68 | C |
| 69 | B | 70 | A |
| 71 | A | 72 | C |
| 73 | C | 74 | A |
| 75 | A | 76 | A |
| 77 | A | | |

In Table 3 and Table 4, Activity represents IC₅₀ value of each Exemplary Compound treatment group to H526 and H69 cell line, respectively (A: <30 nM, B: <50 nM, C: <100 nM, D: <200 nM, E: <400 nM).

### 4. Cell Viability Assay for MRC-5 Cell Line

### (1) Culture of MRC-5 Cell Line

The MRC-5 cell line was purchased from KCLB. Passage of cultured cells was maintained within P15.

For cell culture, MEM/EBSS (Hyclone, Cat. No. SH30024.01; Lot. No. AG29722826), FBS (Gibco), PS (Gibco), 175T cell culture flask (SPL), 96-well cell culture plate (SPL), PBS pH 7.4 (Gibco), TrypLE™ Express (Gibco), Counting Chamber (Thermo Fisher Scientific), and 0.4% Trypan Blue Solution (DYNEBIO) were used.

### (2) Treatment of Compounds of the Present Invention

MRC-5 cell line cultured in 175T cell culture flask was isolated using TrypLE™ Express (Gibco). 6×10³ cells were seeded for each well of a 96-well plate (SPL), and the cells were cultured in total volume of 150 μL.

The compounds of Examples were completely dissolved in DMSO (Sigma-Aldrich) and used in the experiment. Each compound was diluted 3-folds from the highest concentration of 10000 nM to the lowest concentration of 1.52 nM. Each well was mixed with a medium and treated, and the volume was set to 50 μL, so that the total volume of each well was 200 μL. Then, it was cultured in 37° C. CO₂ incubator (Thermo Fisher Scientific) for 5 days.

The following compounds were used as comparative examples, and the cell viability assay was performed in the same manner as in the compounds of Examples.

### Comparative Example 1. Exemplary Compound Described in Mu et al. BBRC, 2020, 521(4): 833 (Comparative Compound 1)

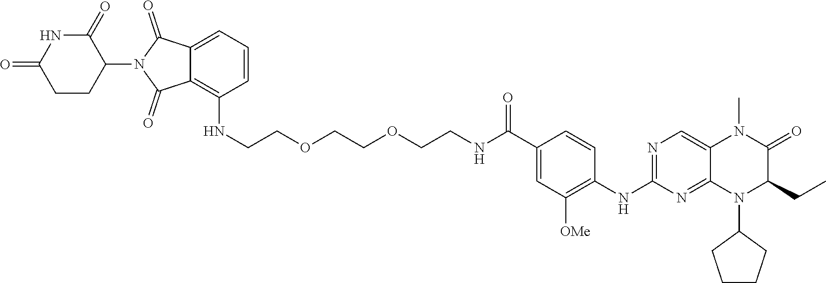 [SMILES: CC[C@@H]1C(=O)N(C)c2cnc(Nc3ccc(C(=O)NCCOCCOCCNc4cccc5c4C(=O)N(C4CCC(=O)NC4=O)C5=O)cc3OC)nc2N1C1CCCC1]

### Comparative Example 2. BI2536 (Comparative Compound 2)

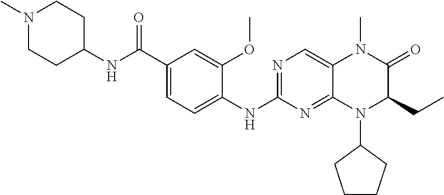 [SMILES: CC[C@@H]1C(=O)N(C)c2cnc(Nc3ccc(C(=O)NC4CCN(C)CC4)cc3OC)nc2N1C1CCCC1]

### Comparative Example 3. Volasertib (Comparative Compound 3)

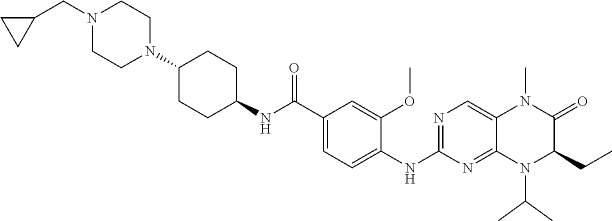 [SMILES: CC[C@@H]1C(=O)N(C)c2cnc(Nc3ccc(C(=O)N[C@H]4CC[C@H](N5CCN(CC6CC6)CC5)CC4)cc3OC)nc2N1C(C)C]

### Comparative Example 4. TAK960 (Comparative Compound 4)

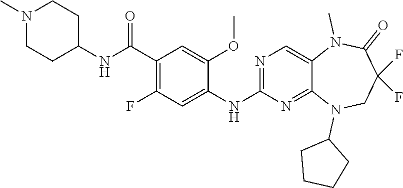 [SMILES: COc1cc(C(=O)NC2CCN(C)CC2)c(F)cc1Nc1ncc2c(n1)N(C1CCCC1)CC(F)(F)C(=O)N2C]

### (3) Cytotoxicity Experiment

After treating EZ-Cytox (DOGEN) 20 μL in each well of completely cultured plate, it was cultured in 37° C. CO₂ incubator for 4 hours. The 96-well plate was placed in a plate reader (BMG Labtech), mixed for 2 minutes, and absorbance was measured at 450 nm wavelength. The data were converted into graphs using the Prism (ver. 9) program.

The results are shown in Table 5 below.

TABLE 5
Cell Viability Assay for MRC-5 cell line

| Exemplary | | Exemplary | |
|---|---|---|---|
| Compound No. | Activity | Compound No. | Activity |
| 1 | N.D. | 2 | N.D. |
| 3 | N.D. | 4 | N.D. |
| 5 | N.D. | 6 | N.D. |
| 7 | N.D. | 8 | N.D. |
| 9 | N.D. | 10 | N.D. |
| 11 | N.D. | 12 | N.D. |
| 13 | N.D. | 14 | N.D. |
| 15 | N.D. | 16 | N.D. |
| 17 | N.D. | 18 | N.D. |
| 19 | N.D. | 20 | N.D. |
| 21 | N.D. | 22 | N.D. |
| 23 | N.D. | 24 | N.D. |
| 25 | N.D. | 26 | N.D. |
| 27 | N.D. | 28 | N.D. |
| 29 | N.D. | 30 | N.D. |
| 32 | N.D. | 33 | N.D. |
| 34 | N.D. | 35 | N.D. |
| 36 | N.D. | 37 | N.D. |
| 38 | N.D. | 39 | N.D. |
| 40 | N.D. | 41 | N.D. |
| 42 | N.D. | 43 | N.D. |
| 44 | N.D. | 45 | N.D. |
| 46 | N.D. | 47 | N.D. |
| 48 | N.D. | 49 | N.D. |
| 50 | N.D. | 51 | N.D. |
| 52 | N.D. | 53 | N.D. |
| 54 | N.D. | 55 | N.D. |
| 56 | N.D. | 57 | N.D. |
| 58 | N.D. | 59 | N.D. |
| 60 | N.D. | 63 | N.D. |
| 64 | N.D. | 65 | N.D. |
| 66 | N.D. | 67 | N.D. |
| 68 | N.D. | 69 | N.D. |
| 71 | N.D. | 72 | N.D. |
| 73 | N.D. | 74 | N.D. |
| 75 | N.D. | 76 | N.D. |
| 77 | N.D. | | |

In Table 5, Activity represents IC₅₀ value (nM) of each Exemplary Compound treatment group to MRC-5 cell line. N.D. (not determined) means that cytotoxicity did not appear until 10 μM As a result, it was confirmed that all of the compounds of the present invention specifically exhibited a high level of cytotoxicity in cancer cell lines rather than normal cell lines.

TABLE 6
Cell Viability Assay for MRC-5 cell line

| Comparative Compound | Activity |
|---|---|
| Comparative Compound 1 | 106.6 |
| Comparative Compound 2 | 3085.4 |
| Comparative Compound 3 | 2939.3 |
| Comparative Compound 4 | 9152.5 |

In Table 6, Activity represents IC₅₀ value (nM) of each Comparative Compound treatment group to MRC-5 cell line. In particular, it was found that Comparative Compound 1, a known PROTAC compound, exhibited a high level of cytotoxicity in normal cell line, unlike the Exemplary Compounds of the present invention.

1. A compound represented by the following Formula I, a stereoisomer thereof or a pharmaceutically acceptable salt thereof: 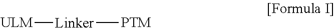
wherein in the Formula I above,
ULM is a moiety represented by the following Formula 1; 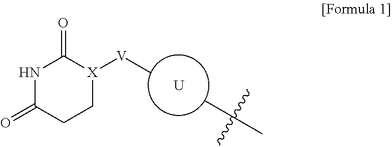
PTM is a moiety represented by the following Formula 2; 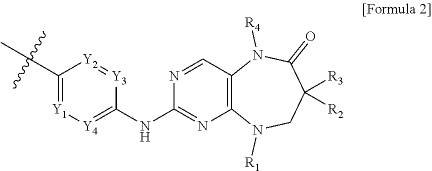
Linker is a group that chemically links ULM and PTM;
X is CH or N;
V is —NRᵥ— or a single bond;
R_{V} is —H or —CH₃;
ring U is phenyl or 5- to 6-membered heteroaryl {wherein at least one H of the phenyl or 5- to 6-membered heteroaryl ring may be substituted with Ru};
R_{U} is —C₁₋₃alkyl, —C₁₋₃hydroxyalkyl, —C₁₋₃aminoalkyl, —C₁₋₃haloalkyl, —C₁₋₃alkoxy, —CN or -halo;
Y₁ is CR_{Y} or N;
Y₂ to Y₄ are each independently CR_{Y};
R_{Y} is —H, —C₁₋₃alkyl, —C₁₋₃alkoxy or -halo;
R₁ is —C₁₋₄alkyl or 5- to 6-membered cycloalkyl;
R₂ and R₃ are each independently —H, —C₁₋₃alkyl or -halo; and
R₄ is —C₁₋₃alkyl.

2. The compound, the stereoisomer thereof or the pharmaceutically acceptable salt thereof according to claim 1, wherein
ULM is a moiety represented by following Formula 1-1, Formula 1-2 or Formula 1-3; 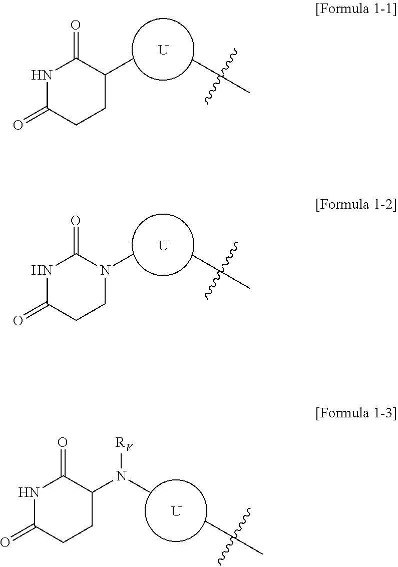
R_{V} is —H or —CH₃,
ring U is phenyl or pyridinyl {wherein at least one H of the phenyl or pyridinyl ring may be substituted with Ru}; and
R_{U} is —C₁₋₃alkyl, —C₁₋₃alkoxy, —CN or -halo.

3. The compound, the stereoisomer thereof or the pharmaceutically acceptable salt thereof according to claim 1, wherein
ULM is 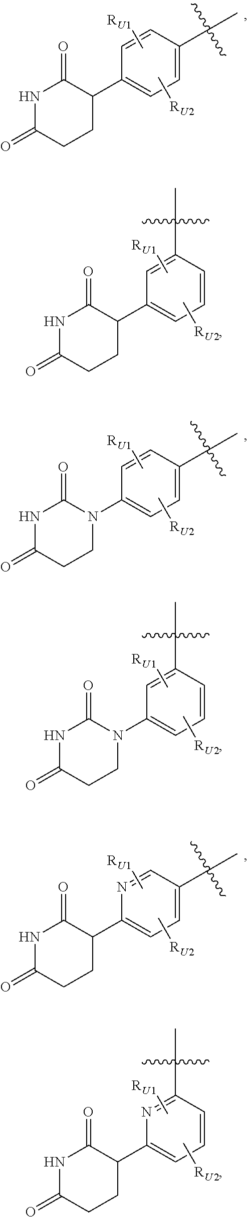 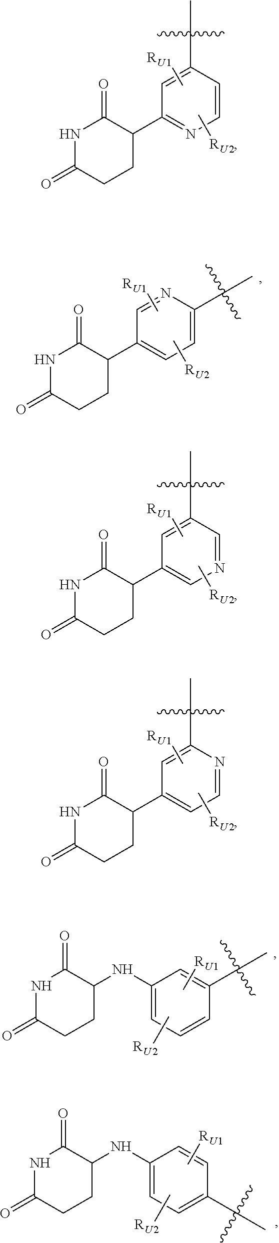 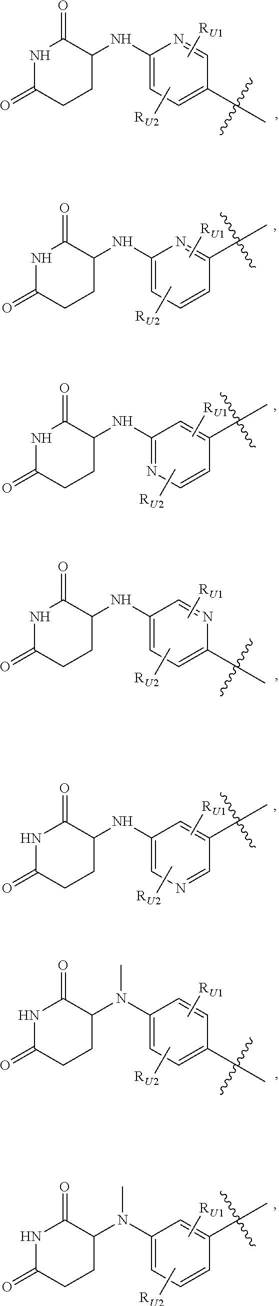 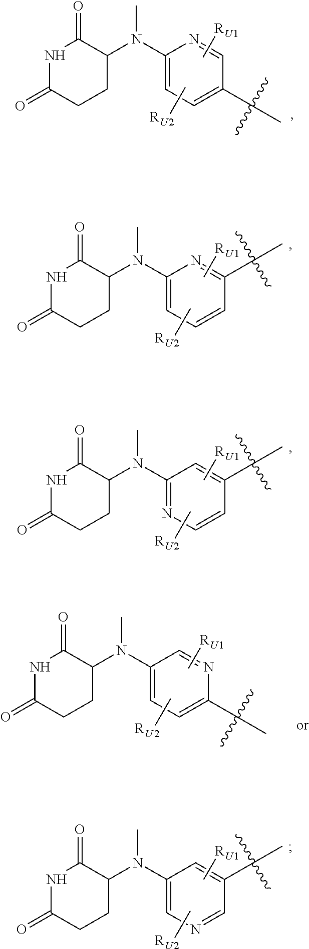
and
R_{U1} and R_{U2} are each independently —C₁₋₃alkyl, —C₁₋₃alkoxy, —CN or -halo.

4. The compound, the stereoisomer thereof or the pharmaceutically acceptable salt thereof according to claim 3, wherein
ULM is 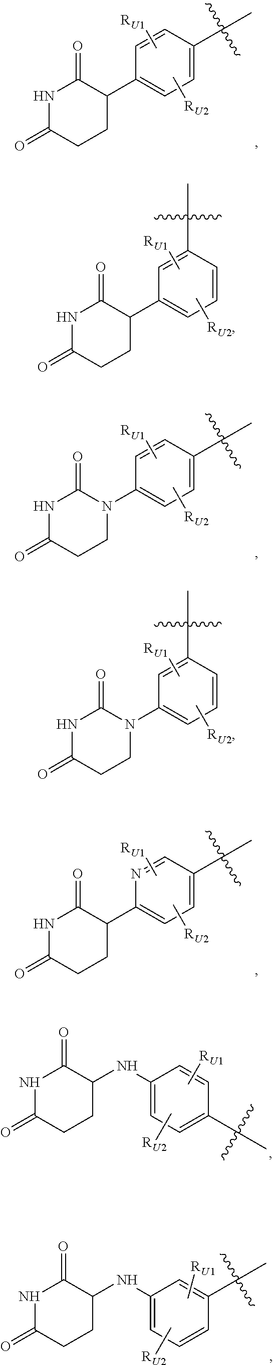 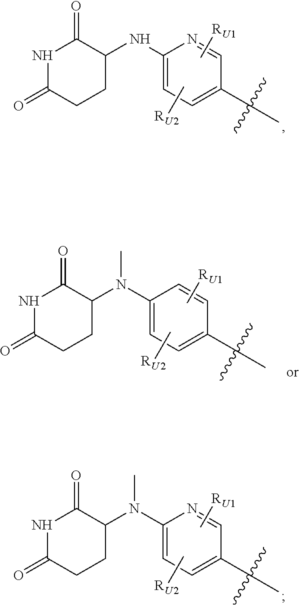
and
R_{U1} and R_{U2} are each independently —C₁₋₃alkyl, —C₁₋₃alkoxy, —CN or -halo.

5. The compound, the stereoisomer thereof or the pharmaceutically acceptable salt thereof according to claim 1, wherein
PTM is 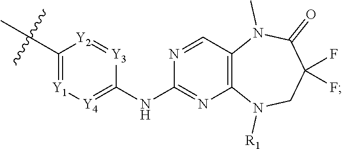
Y₁ is CR_{Y} or N;
Y₂ to Y₄ are each independently CR_{Y};
R_{Y} is —H, —C₁₋₃alkoxy or -halo;
R₁ is —C₁₋₄alkyl or 5- to 6-membered cycloalkyl.

6. The compound, the stereoisomer thereof or the pharmaceutically acceptable salt thereof according to claim 1, wherein
Linker is -L_{U}-L₁-L₂-L_{P}-;
L_{U} is heterocycloalkyl {wherein the heterocycloalkyl contains at least one N atom in the ring, and at least one H of the heterocycloalkyl ring may be substituted with —C₁₋₄alkyl, —C₁₋₄alkoxy or -halo};
L₁ is —(CH₂)_{X1}—, —(CH₂)_{X2}—NH—(CH₂)_{X3}—, —(CH₂)_{X2}—N(C₁₋₄alkyl)-(CH₂)_{X3}— or a single bond {wherein the X1 to X3 are each independently 0, 1, 2, 3 or 4};
L₂ is cycloalkyl or heterocycloalkyl {wherein the heterocycloalkyl contains at least one N atom in the ring, and at least one H of the cycloalkyl or heterocycloalkyl ring may be substituted with —C₁₋₄alkyl, —C₁₋₄alkoxy or -halo}; and
L_{P} is —NH—C(═O)— or —(CH₂)p-heteroaryl {wherein —C(═O)— of the —NH—C(═O)— or heteroaryl of the —(CH₂)p-heteroaryl is linked with PTM, and the heteroaryl contains N and O atoms in the ring, and the p is 0, 1, 2 or 3}.

7. The compound, the stereoisomer thereof or the pharmaceutically acceptable salt thereof according to claim 6, wherein
L_{U} is 4- to 12-membered heterocycloalkyl {wherein the 4- to 12-membered heterocycloalkyl is single ring, bridged bicyclic ring or spiro ring, the 4- to 12-membered heterocycloalkyl contains at least one N atom in the ring, the N atom is directly linked with ULM, and at least one H of the 4- to 12-membered heterocycloalkyl ring may be substituted with —C₁₋₄alkyl or -halo};
L_{U} is —(CH₂)_{X1}—, —(CH₂)_{X2}—N(C₁₋₄alkyl)-(CH₂)_{X3}— or a single bond {wherein the X1 to X3 are each independently 0, 1, 2 or 3}
L₂ is 4- to 6-membered cycloalkyl or 4- to 12-membered heterocycloalkyl {wherein the 4- to 12-membered heterocycloalkyl is single ring, bridged bicyclic ring or spiro ring, the 4- to 12-membered heterocycloalkyl contains at least one N atom in the ring, and at least one H of the 4- to 6-membered cycloalkyl or 4- to 12-membered heterocycloalkyl ring may be substituted with —C₁₋₄alkyl or -halo}; and
L_{P} is —NH—C(═O)— or —(CH₂)p-1,3,4-oxadiazol {wherein —C(═O)— of the —NH—C(═O)— or 1,3,4-oxadiazol of the —(CH₂)p-1,3,4-oxadiazol is linked with PTM, and the p is 0 or 1}.

8. The compound, the stereoisomer thereof or the pharmaceutically acceptable salt thereof according to claim 1, wherein the compound represented by the Formula I is selected from:

| Compound | Structure |
|---|---|
| 1 | 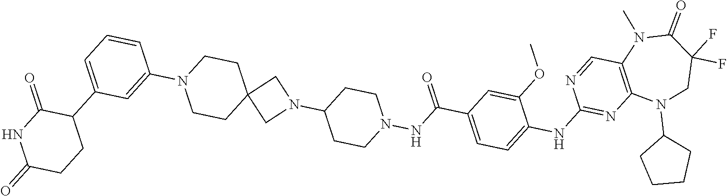 [SMILES: COc1cc(C(=O)NN2CCC(N3CC4(CCN(c5cccc(C6CCC(=O)NC6=O)c5)CC4)C3)CC2)ccc1Nc1ncc2c(n1)N(C1CCCC1)CC(F)(F)C(=O)N2C] |
| 2 | 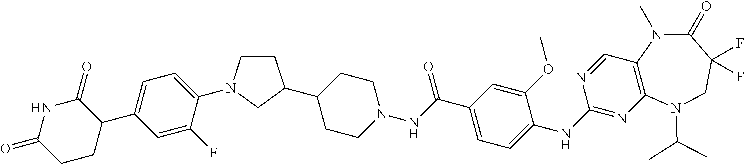 [SMILES: COc1cc(C(=O)NN2CCC(C3CCN(c4ccc(C5CCC(=O)NC5=O)cc4F)C3)CC2)ccc1Nc1ncc2c(n1)N(C(C)C)CC(F)(F)C(=O)N2C] |
| 3 | 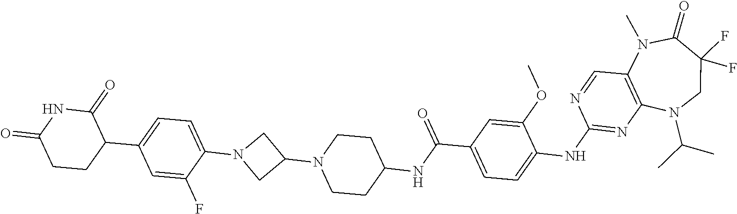 [SMILES: COc1cc(C(=O)NC2CCN(C3CN(c4ccc(C5CCC(=O)NC5=O)cc4F)C3)CC2)ccc1Nc1ncc2c(n1)N(C(C)C)CC(F)(F)C(=O)N2C] |
| 4 | 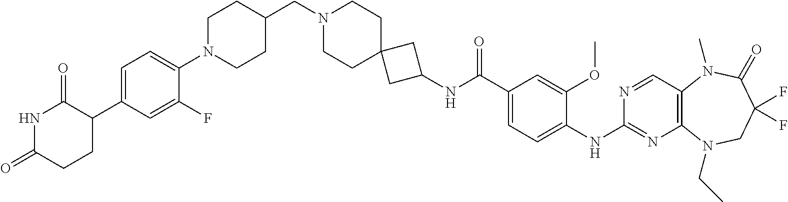 [SMILES: CCN1CC(F)(F)C(=O)N(C)c2cnc(Nc3ccc(C(=O)NC4CC5(CCN(CC6CCN(c7ccc(C8CCC(=O)NC8=O)cc7F)CC6)CC5)C4)cc3OC)nc21] |
| 5 | 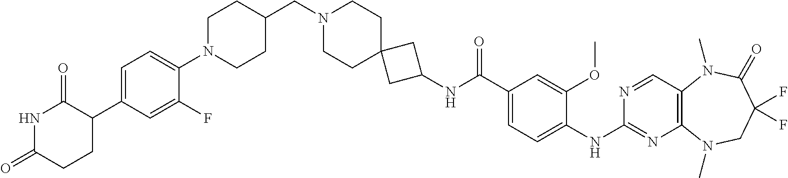 [SMILES: COc1cc(C(=O)NC2CC3(CCN(CC4CCN(c5ccc(C6CCC(=O)NC6=O)cc5F)CC4)CC3)C2)ccc1Nc1ncc2c(n1)N(C)CC(F)(F)C(=O)N2C] |
| 6 | 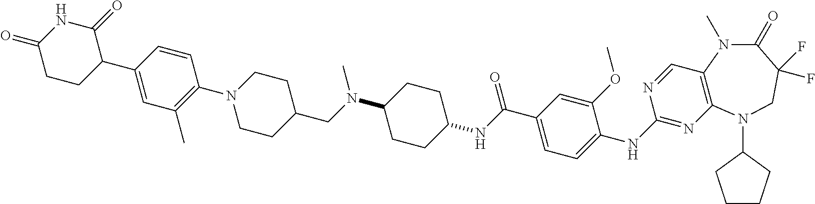 |
| 7 | 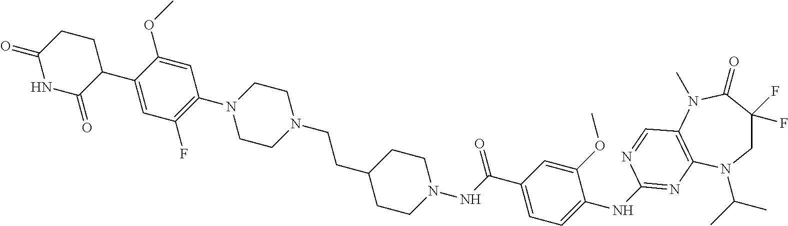 [SMILES: COc1cc(C(=O)NN2CCC(CCN3CCN(c4cc(OC)c(C5CCC(=O)NC5=O)cc4F)CC3)CC2)ccc1Nc1ncc2c(n1)N(C(C)C)CC(F)(F)C(=O)N2C] |
| 8 | 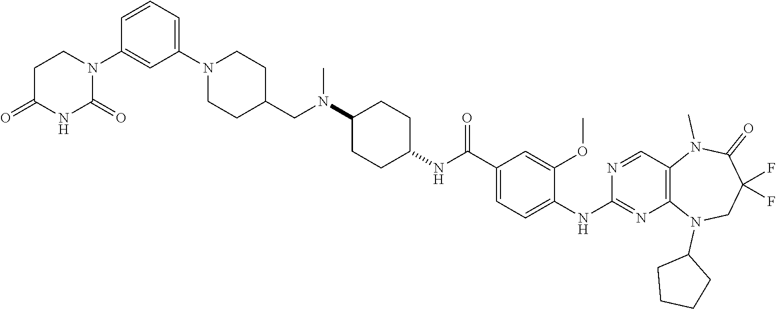 [SMILES: COc1cc(C(=O)N[C@H]2CC[C@H](N(C)CC3CCN(c4cccc(N5CCC(=O)NC5=O)c4)CC3)CC2)ccc1Nc1ncc2c(n1)N(C1CCCC1)CC(F)(F)C(=O)N2C] |
| 9 | 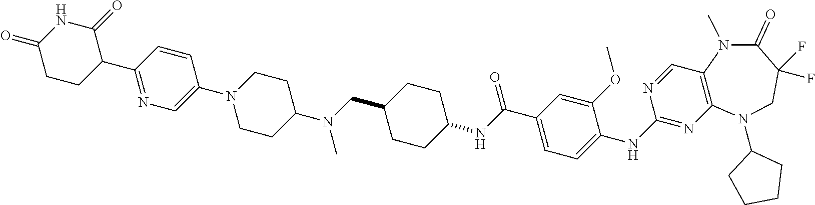 [SMILES: COc1cc(C(=O)N[C@H]2CC[C@H](CN(C)C3CCN(c4ccc(C5CCC(=O)NC5=O)nc4)CC3)CC2)ccc1Nc1ncc2c(n1)N(C1CCCC1)CC(F)(F)C(=O)N2C] |
| 10 | 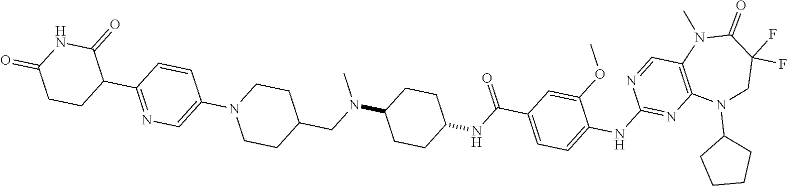 |
| 11 | 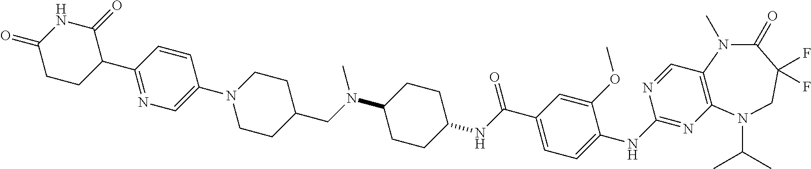 |
| 12 | 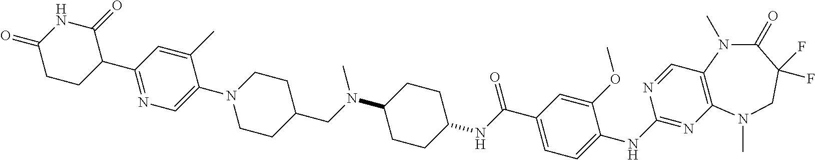 |
| 13 | 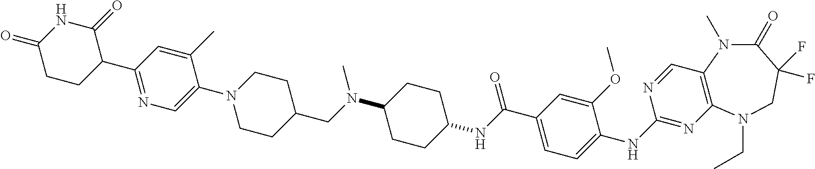 |
| 14 | 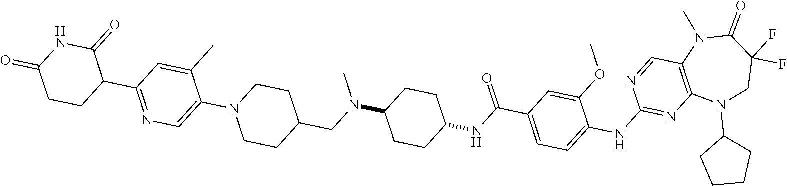 |
| 15 | 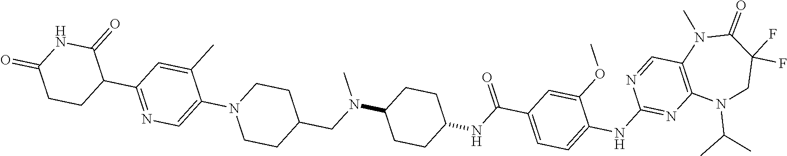 |
| 16 | 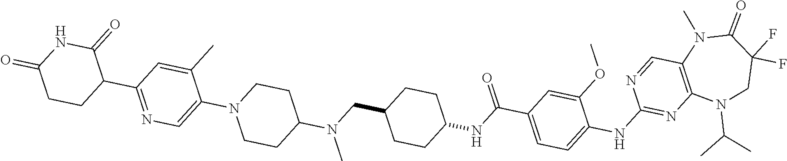 |
| 17 | 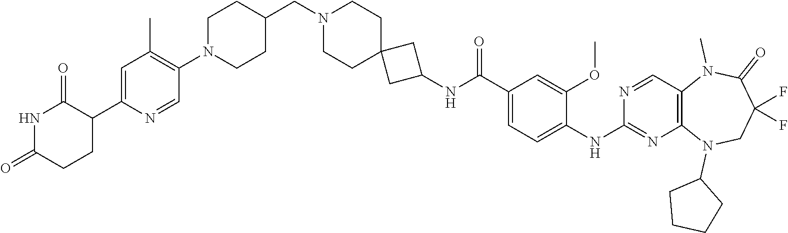 [SMILES: COc1cc(C(=O)NC2CC3(CCN(CC4CCN(c5cnc(C6CCC(=O)NC6=O)cc5C)CC4)CC3)C2)ccc1Nc1ncc2c(n1)N(C1CCCC1)CC(F)(F)C(=O)N2C] |
| 18 | 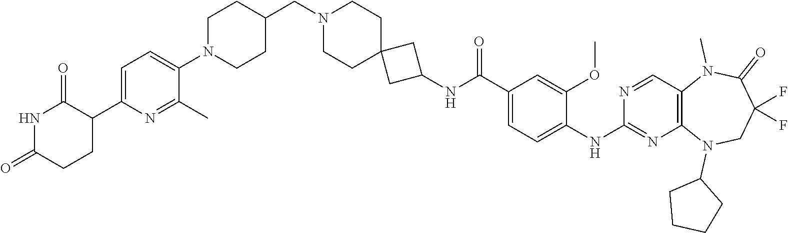 [SMILES: COc1cc(C(=O)NC2CC3(CCN(CC4CCN(c5ccc(C6CCC(=O)NC6=O)nc5C)CC4)CC3)C2)ccc1Nc1ncc2c(n1)N(C1CCCC1)CC(F)(F)C(=O)N2C] |
| 19 | 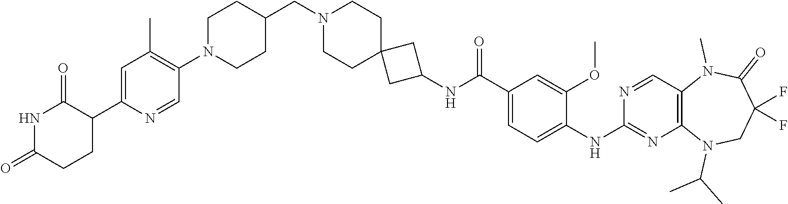 [SMILES: COc1cc(C(=O)NC2CC3(CCN(CC4CCN(c5cnc(C6CCC(=O)NC6=O)cc5C)CC4)CC3)C2)ccc1Nc1ncc2c(n1)N(C(C)C)CC(F)(F)C(=O)N2C] |
| 20 | 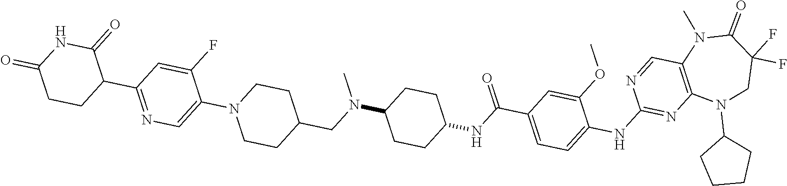 |
| 21 | 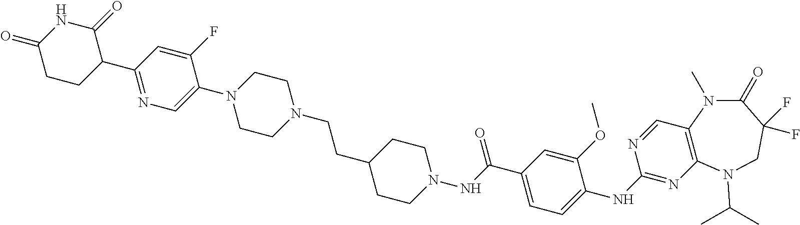 [SMILES: COc1cc(C(=O)NN2CCC(CCN3CCN(c4cnc(C5CCC(=O)NC5=O)cc4F)CC3)CC2)ccc1Nc1ncc2c(n1)N(C(C)C)CC(F)(F)C(=O)N2C] |
| 22 | 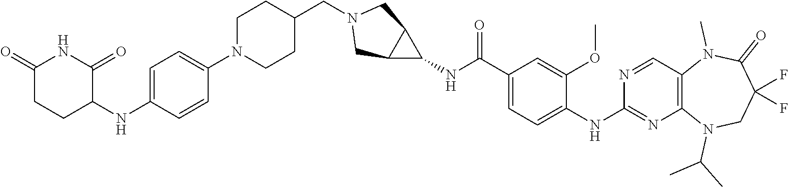 [SMILES: COc1cc(C(=O)N[C@H]2[C@@H]3CN(CC4CCN(c5ccc(NC6CCC(=O)NC6=O)cc5)CC4)C[C@@H]32)ccc1Nc1ncc2c(n1)N(C(C)C)CC(F)(F)C(=O)N2C] |
| 23 | 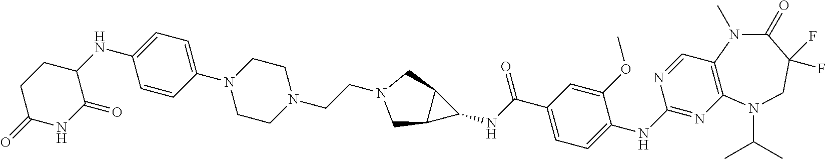 [SMILES: COc1cc(C(=O)N[C@H]2[C@@H]3CN(CCN4CCN(c5ccc(NC6CCC(=O)NC6=O)cc5)CC4)C[C@@H]32)ccc1Nc1ncc2c(n1)N(C(C)C)CC(F)(F)C(=O)N2C] |
| 24 | 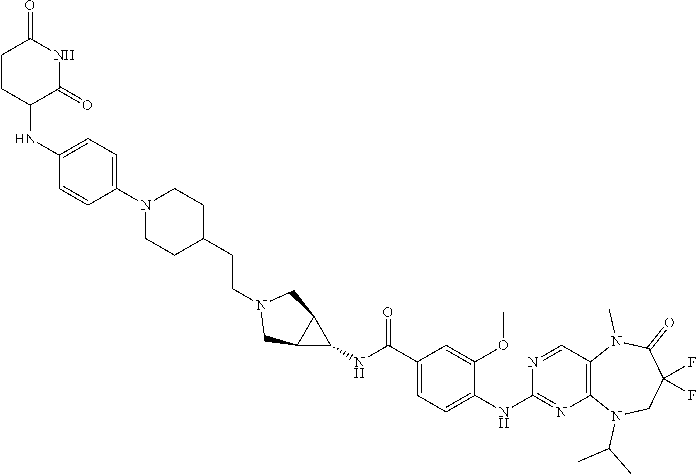 [SMILES: COc1cc(C(=O)N[C@H]2[C@@H]3CN(CCC4CCN(c5ccc(NC6CCC(=O)NC6=O)cc5)CC4)C[C@@H]32)ccc1Nc1ncc2c(n1)N(C(C)C)CC(F)(F)C(=O)N2C] |
| 25 | 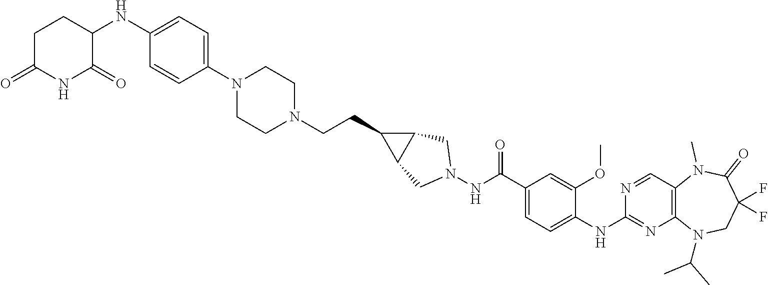 [SMILES: COc1cc(C(=O)NN2C[C@@H]3[C@@H](CCN4CCN(c5ccc(NC6CCC(=O)NC6=O)cc5)CC4)[C@@H]3C2)ccc1Nc1ncc2c(n1)N(C(C)C)CC(F)(F)C(=O)N2C] |
| 26 | 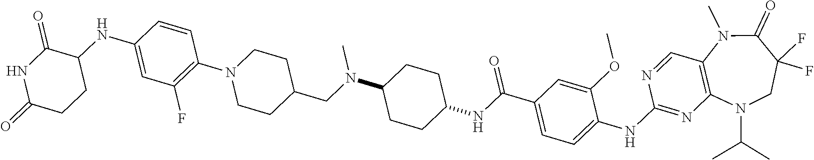 |
| 27 | 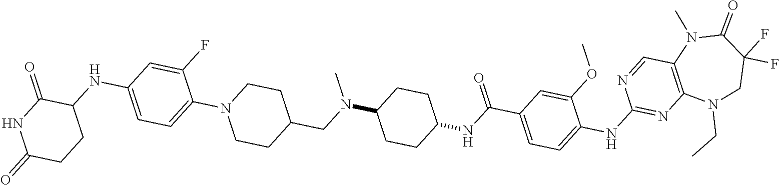 |
| 28 | 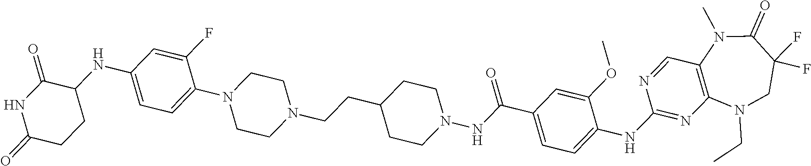 [SMILES: CCN1CC(F)(F)C(=O)N(C)c2cnc(Nc3ccc(C(=O)NN4CCC(CCN5CCN(c6ccc(NC7CCC(=O)NC7=O)cc6F)CC5)CC4)cc3OC)nc21] |
| 29 | 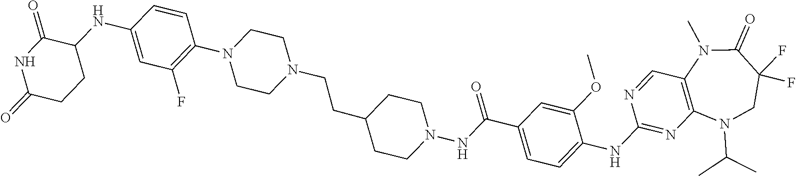 [SMILES: COc1cc(C(=O)NN2CCC(CCN3CCN(c4ccc(NC5CCC(=O)NC5=O)cc4F)CC3)CC2)ccc1Nc1ncc2c(n1)N(C(C)C)CC(F)(F)C(=O)N2C] |
| 30 | 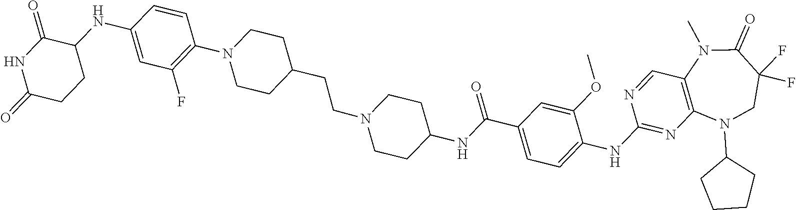 [SMILES: COc1cc(C(=O)NC2CCN(CCC3CCN(c4ccc(NC5CCC(=O)NC5=O)cc4F)CC3)CC2)ccc1Nc1ncc2c(n1)N(C1CCCC1)CC(F)(F)C(=O)N2C] |
| 31 | 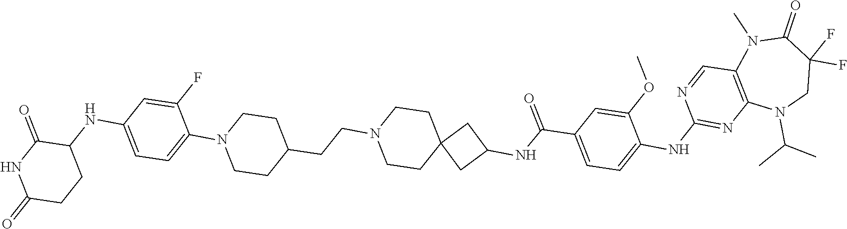 [SMILES: COc1cc(C(=O)NC2CC3(CCN(CCC4CCN(c5ccc(NC6CCC(=O)NC6=O)cc5F)CC4)CC3)C2)ccc1Nc1ncc2c(n1)N(C(C)C)CC(F)(F)C(=O)N2C] |
| 32 | 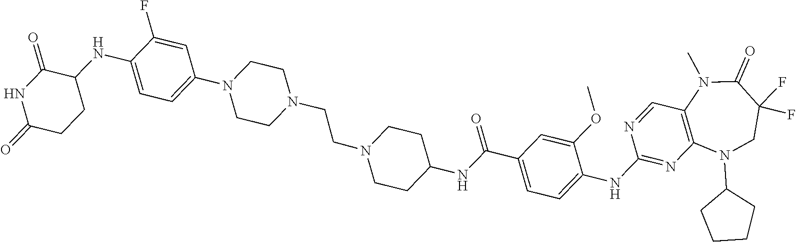 [SMILES: COc1cc(C(=O)NC2CCN(CCN3CCN(c4ccc(NC5CCC(=O)NC5=O)c(F)c4)CC3)CC2)ccc1Nc1ncc2c(n1)N(C1CCCC1)CC(F)(F)C(=O)N2C] |
| 33 | 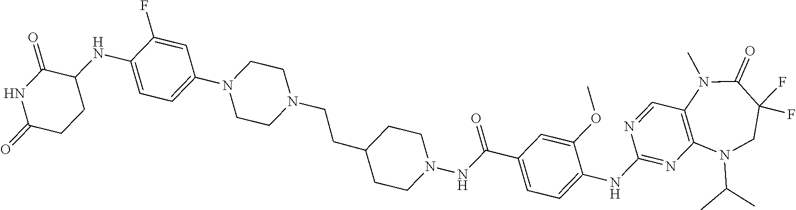 [SMILES: COc1cc(C(=O)NN2CCC(CCN3CCN(c4ccc(NC5CCC(=O)NC5=O)c(F)c4)CC3)CC2)ccc1Nc1ncc2c(n1)N(C(C)C)CC(F)(F)C(=O)N2C] |
| 34 | 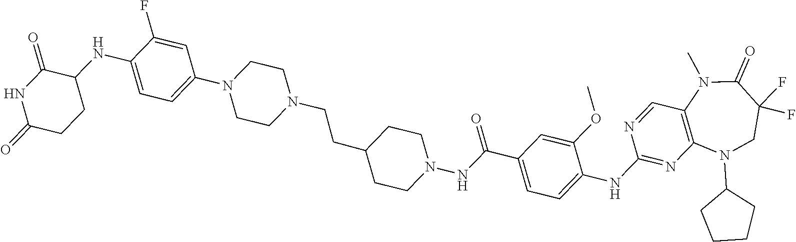 [SMILES: COc1cc(C(=O)NN2CCC(CCN3CCN(c4ccc(NC5CCC(=O)NC5=O)c(F)c4)CC3)CC2)ccc1Nc1ncc2c(n1)N(C1CCCC1)CC(F)(F)C(=O)N2C] |
| 35 | 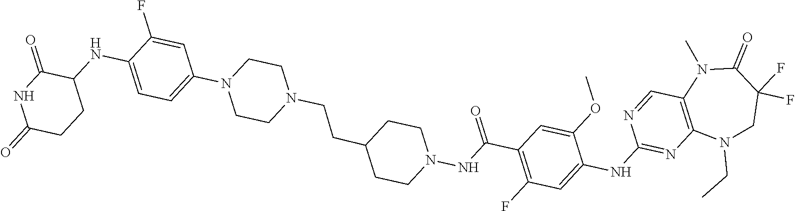 [SMILES: CCN1CC(F)(F)C(=O)N(C)c2cnc(Nc3cc(F)c(C(=O)NN4CCC(CCN5CCN(c6ccc(NC7CCC(=O)NC7=O)c(F)c6)CC5)CC4)cc3OC)nc21] |
| 36 | 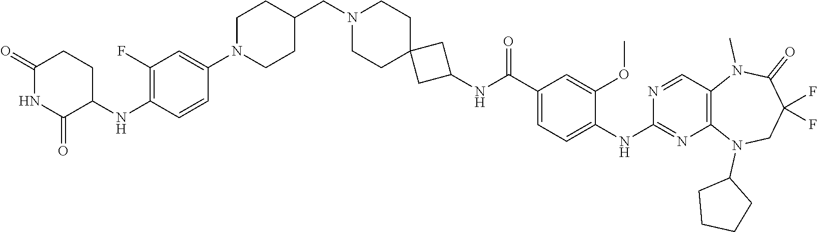 [SMILES: COc1cc(C(=O)NC2CC3(CCN(CC4CCN(c5ccc(NC6CCC(=O)NC6=O)c(F)c5)CC4)CC3)C2)ccc1Nc1ncc2c(n1)N(C1CCCC1)CC(F)(F)C(=O)N2C] |
| 37 | 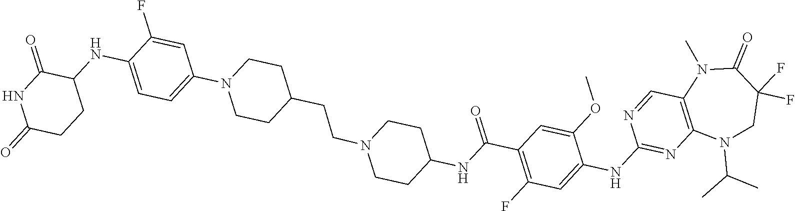 [SMILES: COc1cc(C(=O)NC2CCN(CCC3CCN(c4ccc(NC5CCC(=O)NC5=O)c(F)c4)CC3)CC2)c(F)cc1Nc1ncc2c(n1)N(C(C)C)CC(F)(F)C(=O)N2C] |
| 38 | 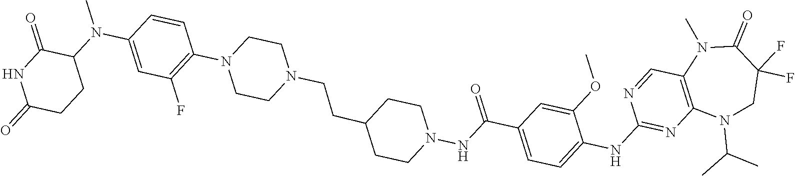 [SMILES: COc1cc(C(=O)NN2CCC(CCN3CCN(c4ccc(N(C)C5CCC(=O)NC5=O)cc4F)CC3)CC2)ccc1Nc1ncc2c(n1)N(C(C)C)CC(F)(F)C(=O)N2C] |
| 39 | 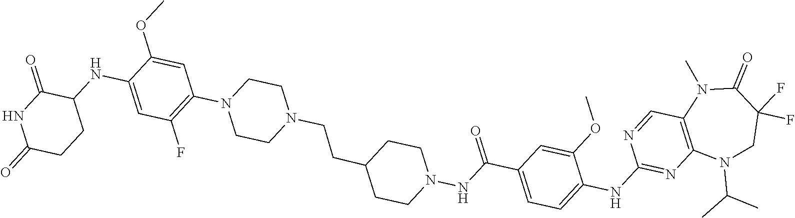 [SMILES: COc1cc(C(=O)NN2CCC(CCN3CCN(c4cc(OC)c(NC5CCC(=O)NC5=O)cc4F)CC3)CC2)ccc1Nc1ncc2c(n1)N(C(C)C)CC(F)(F)C(=O)N2C] |
| 40 | 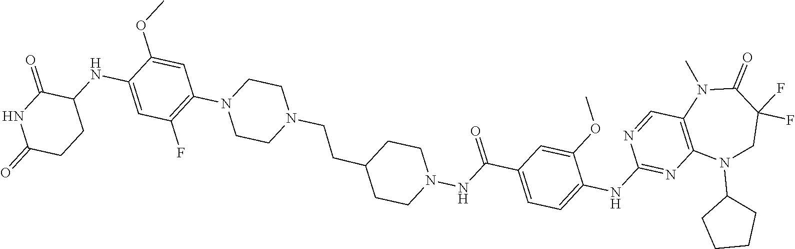 [SMILES: COc1cc(C(=O)NN2CCC(CCN3CCN(c4cc(OC)c(NC5CCC(=O)NC5=O)cc4F)CC3)CC2)ccc1Nc1ncc2c(n1)N(C1CCCC1)CC(F)(F)C(=O)N2C] |
| 41 | 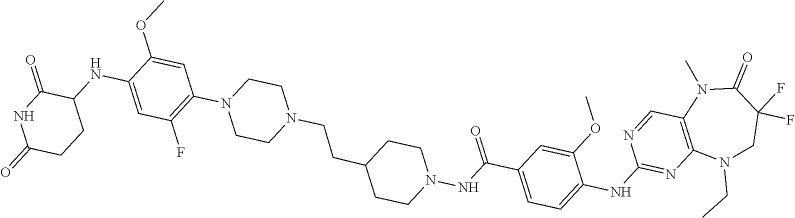 [SMILES: CCN1CC(F)(F)C(=O)N(C)c2cnc(Nc3ccc(C(=O)NN4CCC(CCN5CCN(c6cc(OC)c(NC7CCC(=O)NC7=O)cc6F)CC5)CC4)cc3OC)nc21] |
| 42 | 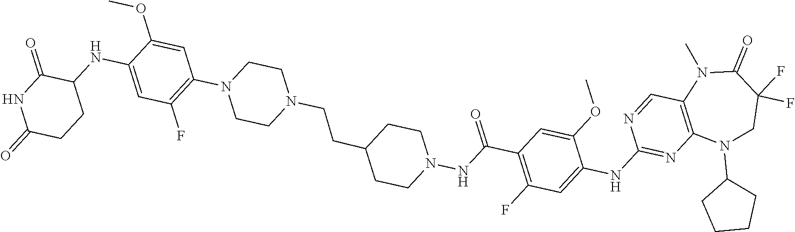 [SMILES: COc1cc(C(=O)NN2CCC(CCN3CCN(c4cc(OC)c(NC5CCC(=O)NC5=O)cc4F)CC3)CC2)c(F)cc1Nc1ncc2c(n1)N(C1CCCC1)CC(F)(F)C(=O)N2C] |
| 43 | 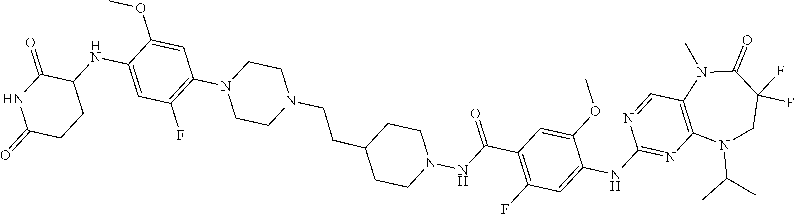 [SMILES: COc1cc(C(=O)NN2CCC(CCN3CCN(c4cc(OC)c(NC5CCC(=O)NC5=O)cc4F)CC3)CC2)c(F)cc1Nc1ncc2c(n1)N(C(C)C)CC(F)(F)C(=O)N2C] |
| 44 | 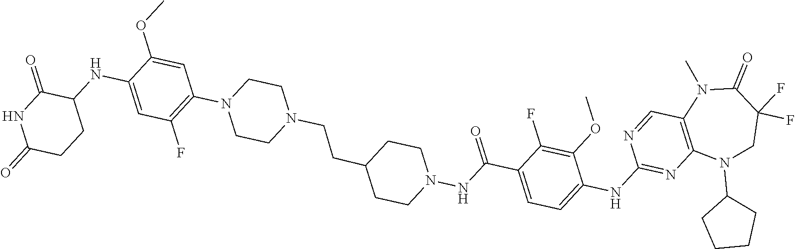 [SMILES: COc1cc(N2CCN(CCC3CCN(NC(=O)c4ccc(Nc5ncc6c(n5)N(C5CCCC5)CC(F)(F)C(=O)N6C)c(OC)c4F)CC3)CC2)c(F)cc1NC1CCC(=O)NC1=O] |
| 45 | 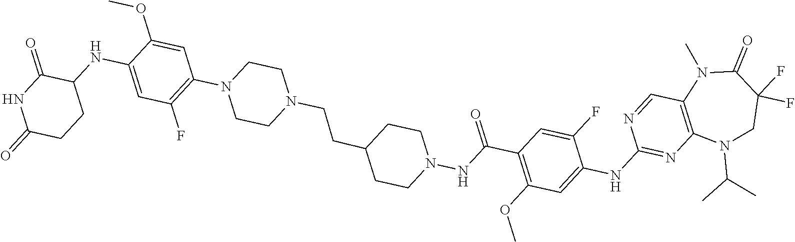 [SMILES: COc1cc(N2CCN(CCC3CCN(NC(=O)c4cc(F)c(Nc5ncc6c(n5)N(C(C)C)CC(F)(F)C(=O)N6C)cc4OC)CC3)CC2)c(F)cc1NC1CCC(=O)NC1=O] |
| 46 | 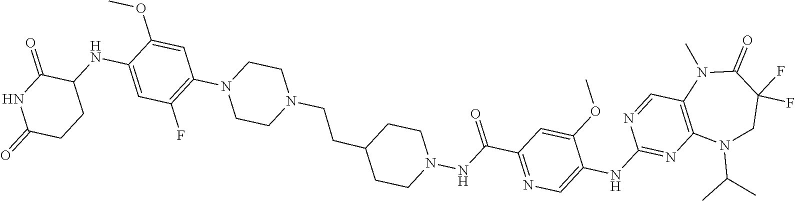 [SMILES: COc1cc(C(=O)NN2CCC(CCN3CCN(c4cc(OC)c(NC5CCC(=O)NC5=O)cc4F)CC3)CC2)ncc1Nc1ncc2c(n1)N(C(C)C)CC(F)(F)C(=O)N2C] |
| 47 | 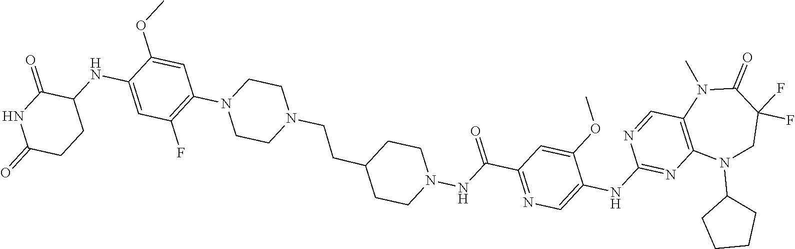 [SMILES: COc1cc(C(=O)NN2CCC(CCN3CCN(c4cc(OC)c(NC5CCC(=O)NC5=O)cc4F)CC3)CC2)ncc1Nc1ncc2c(n1)N(C1CCCC1)CC(F)(F)C(=O)N2C] |
| 48 | 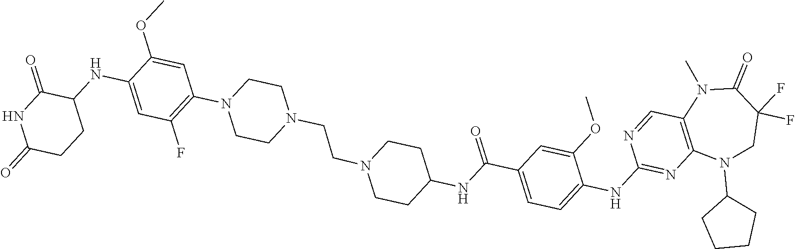 [SMILES: COc1cc(C(=O)NC2CCN(CCN3CCN(c4cc(OC)c(NC5CCC(=O)NC5=O)cc4F)CC3)CC2)ccc1Nc1ncc2c(n1)N(C1CCCC1)CC(F)(F)C(=O)N2C] |
| 49 | 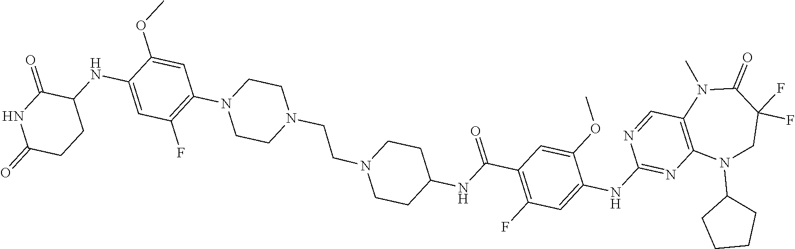 [SMILES: COc1cc(C(=O)NC2CCN(CCN3CCN(c4cc(OC)c(NC5CCC(=O)NC5=O)cc4F)CC3)CC2)c(F)cc1Nc1ncc2c(n1)N(C1CCCC1)CC(F)(F)C(=O)N2C] |
| 50 | 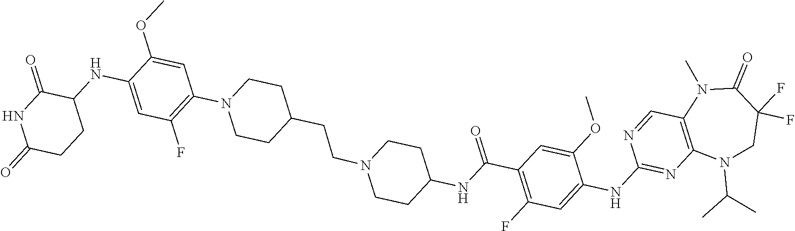 [SMILES: COc1cc(C(=O)NC2CCN(CCC3CCN(c4cc(OC)c(NC5CCC(=O)NC5=O)cc4F)CC3)CC2)c(F)cc1Nc1ncc2c(n1)N(C(C)C)CC(F)(F)C(=O)N2C] |
| 51 | 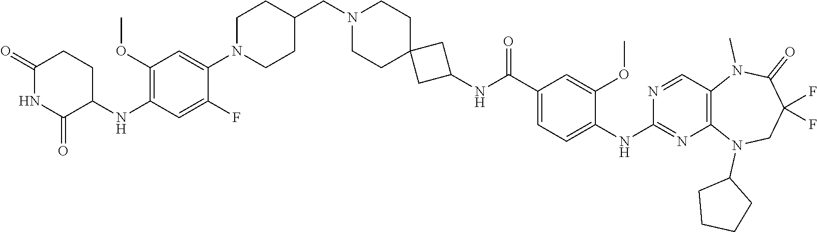 [SMILES: COc1cc(C(=O)NC2CC3(CCN(CC4CCN(c5cc(OC)c(NC6CCC(=O)NC6=O)cc5F)CC4)CC3)C2)ccc1Nc1ncc2c(n1)N(C1CCCC1)CC(F)(F)C(=O)N2C] |
| 52 | 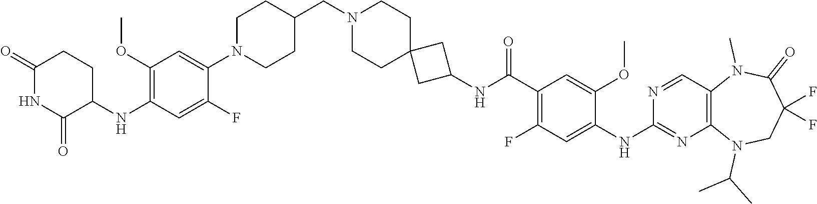 [SMILES: COc1cc(C(=O)NC2CC3(CCN(CC4CCN(c5cc(OC)c(NC6CCC(=O)NC6=O)cc5F)CC4)CC3)C2)c(F)cc1Nc1ncc2c(n1)N(C(C)C)CC(F)(F)C(=O)N2C] |
| 53 | 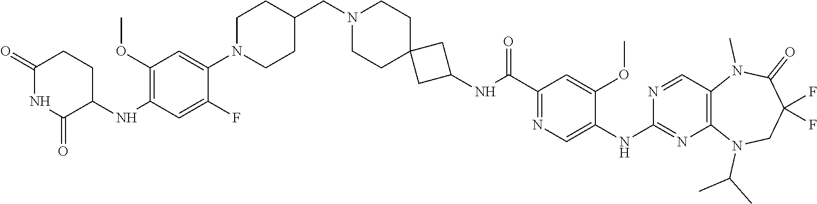 [SMILES: COc1cc(C(=O)NC2CC3(CCN(CC4CCN(c5cc(OC)c(NC6CCC(=O)NC6=O)cc5F)CC4)CC3)C2)ncc1Nc1ncc2c(n1)N(C(C)C)CC(F)(F)C(=O)N2C] |
| 54 | 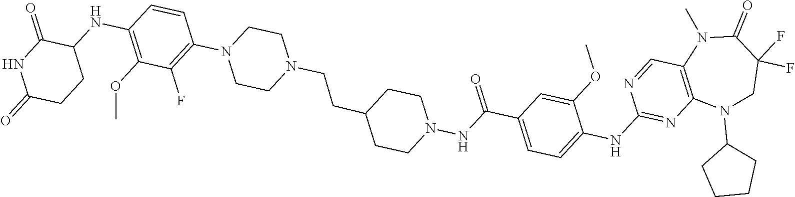 [SMILES: COc1cc(C(=O)NN2CCC(CCN3CCN(c4ccc(NC5CCC(=O)NC5=O)c(OC)c4F)CC3)CC2)ccc1Nc1ncc2c(n1)N(C1CCCC1)CC(F)(F)C(=O)N2C] |
| 55 | 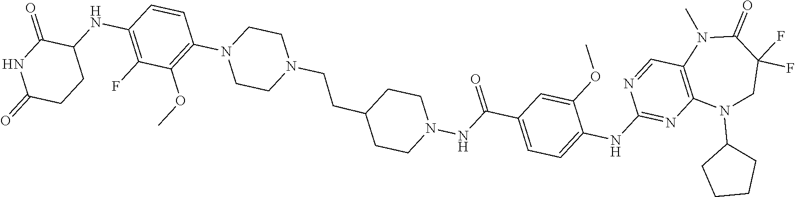 [SMILES: COc1cc(C(=O)NN2CCC(CCN3CCN(c4ccc(NC5CCC(=O)NC5=O)c(F)c4OC)CC3)CC2)ccc1Nc1ncc2c(n1)N(C1CCCC1)CC(F)(F)C(=O)N2C] |
| 56 | 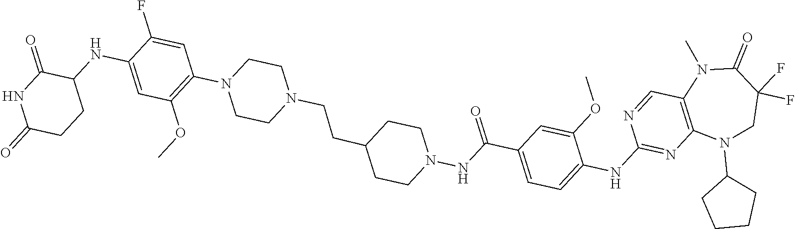 [SMILES: COc1cc(C(=O)NN2CCC(CCN3CCN(c4cc(F)c(NC5CCC(=O)NC5=O)cc4OC)CC3)CC2)ccc1Nc1ncc2c(n1)N(C1CCCC1)CC(F)(F)C(=O)N2C] |
| 57 | 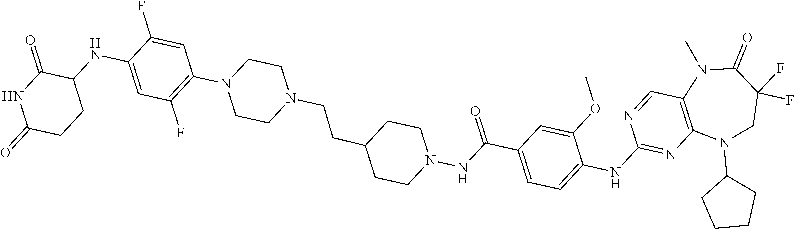 [SMILES: COc1cc(C(=O)NN2CCC(CCN3CCN(c4cc(F)c(NC5CCC(=O)NC5=O)cc4F)CC3)CC2)ccc1Nc1ncc2c(n1)N(C1CCCC1)CC(F)(F)C(=O)N2C] |
| 58 | 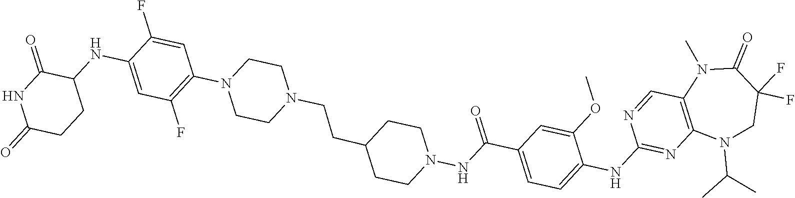 [SMILES: COc1cc(C(=O)NN2CCC(CCN3CCN(c4cc(F)c(NC5CCC(=O)NC5=O)cc4F)CC3)CC2)ccc1Nc1ncc2c(n1)N(C(C)C)CC(F)(F)C(=O)N2C] |
| 59 | 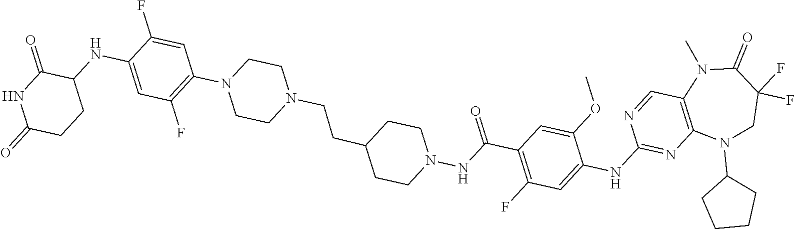 [SMILES: COc1cc(C(=O)NN2CCC(CCN3CCN(c4cc(F)c(NC5CCC(=O)NC5=O)cc4F)CC3)CC2)c(F)cc1Nc1ncc2c(n1)N(C1CCCC1)CC(F)(F)C(=O)N2C] |
| 60 | 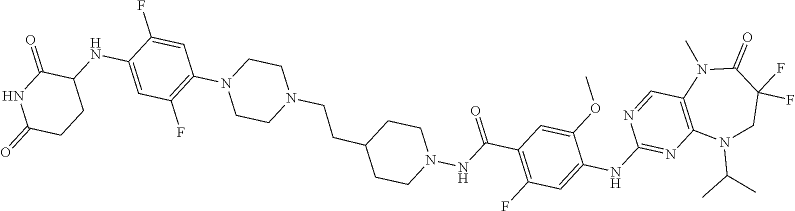 [SMILES: COc1cc(C(=O)NN2CCC(CCN3CCN(c4cc(F)c(NC5CCC(=O)NC5=O)cc4F)CC3)CC2)c(F)cc1Nc1ncc2c(n1)N(C(C)C)CC(F)(F)C(=O)N2C] |
| 61 | 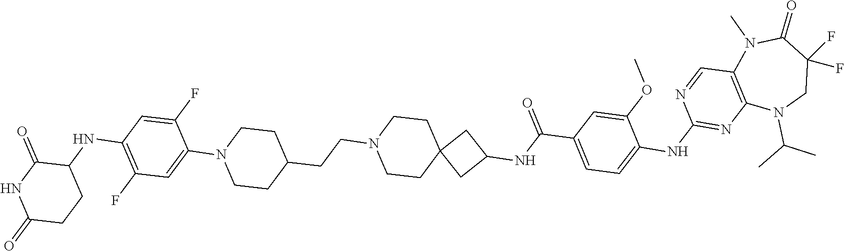 [SMILES: COc1cc(C(=O)NC2CC3(CCN(CCC4CCN(c5cc(F)c(NC6CCC(=O)NC6=O)cc5F)CC4)CC3)C2)ccc1Nc1ncc2c(n1)N(C(C)C)CC(F)(F)C(=O)N2C] |
| 62 | 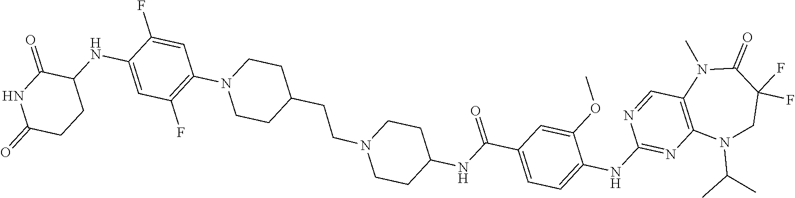 [SMILES: COc1cc(C(=O)NC2CCN(CCC3CCN(c4cc(F)c(NC5CCC(=O)NC5=O)cc4F)CC3)CC2)ccc1Nc1ncc2c(n1)N(C(C)C)CC(F)(F)C(=O)N2C] |
| 63 | 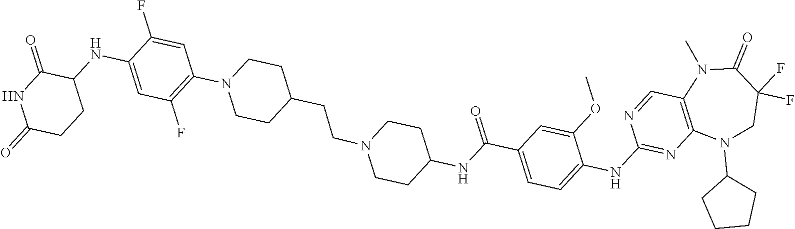 [SMILES: COc1cc(C(=O)NC2CCN(CCC3CCN(c4cc(F)c(NC5CCC(=O)NC5=O)cc4F)CC3)CC2)ccc1Nc1ncc2c(n1)N(C1CCCC1)CC(F)(F)C(=O)N2C] |
| 64 | 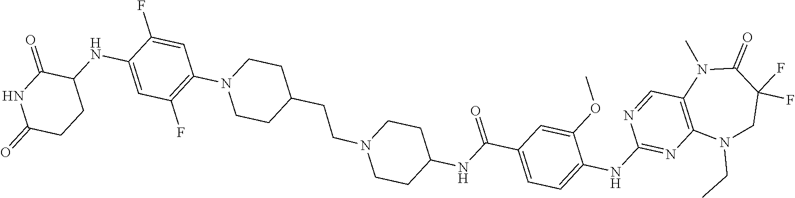 [SMILES: CCN1CC(F)(F)C(=O)N(C)c2cnc(Nc3ccc(C(=O)NC4CCN(CCC5CCN(c6cc(F)c(NC7CCC(=O)NC7=O)cc6F)CC5)CC4)cc3OC)nc21] |
| 65 | 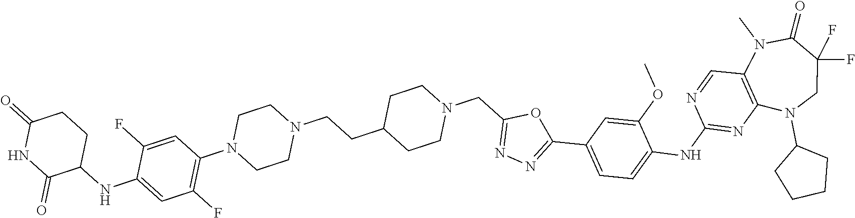 [SMILES: COc1cc(-c2nnc(CN3CCC(CCN4CCN(c5cc(F)c(NC6CCC(=O)NC6=O)cc5F)CC4)CC3)o2)ccc1Nc1ncc2c(n1)N(C1CCCC1)CC(F)(F)C(=O)N2C] |
| 66 | 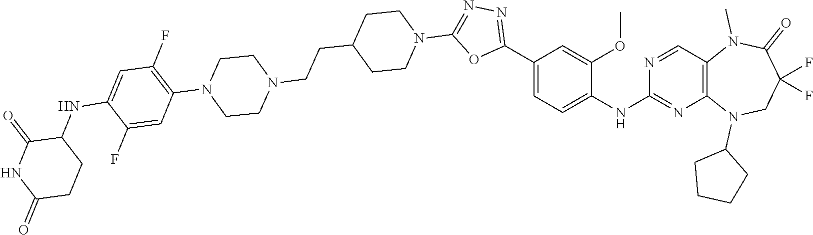 [SMILES: COc1cc(-c2nnc(N3CCC(CCN4CCN(c5cc(F)c(NC6CCC(=O)NC6=O)cc5F)CC4)CC3)o2)ccc1Nc1ncc2c(n1)N(C1CCCC1)CC(F)(F)C(=O)N2C] |
| 67 | 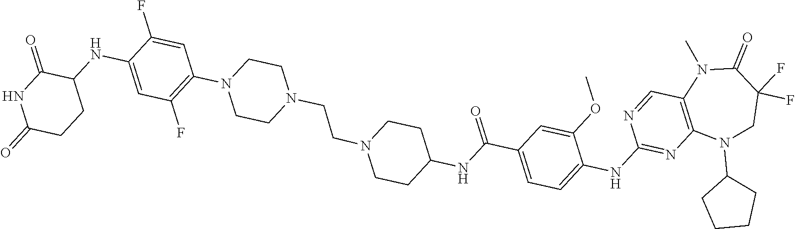 [SMILES: COc1cc(C(=O)NC2CCN(CCN3CCN(c4cc(F)c(NC5CCC(=O)NC5=O)cc4F)CC3)CC2)ccc1Nc1ncc2c(n1)N(C1CCCC1)CC(F)(F)C(=O)N2C] |
| 68 | 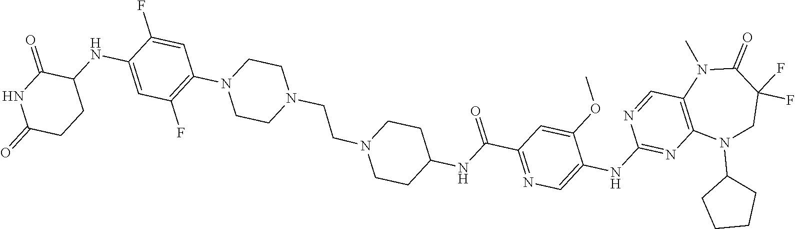 [SMILES: COc1cc(C(=O)NC2CCN(CCN3CCN(c4cc(F)c(NC5CCC(=O)NC5=O)cc4F)CC3)CC2)ncc1Nc1ncc2c(n1)N(C1CCCC1)CC(F)(F)C(=O)N2C] |
| 69 | 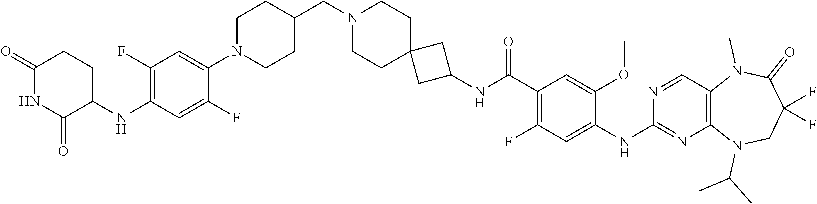 [SMILES: COc1cc(C(=O)NC2CC3(CCN(CC4CCN(c5cc(F)c(NC6CCC(=O)NC6=O)cc5F)CC4)CC3)C2)c(F)cc1Nc1ncc2c(n1)N(C(C)C)CC(F)(F)C(=O)N2C] |
| 70 | 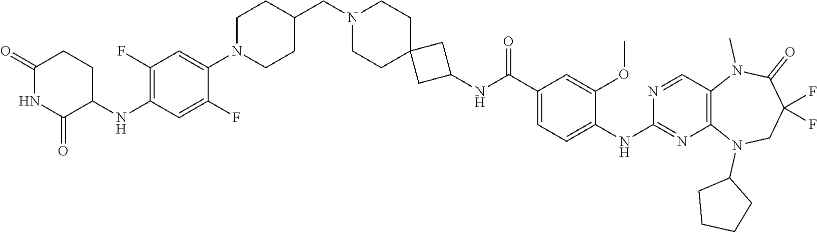 [SMILES: COc1cc(C(=O)NC2CC3(CCN(CC4CCN(c5cc(F)c(NC6CCC(=O)NC6=O)cc5F)CC4)CC3)C2)ccc1Nc1ncc2c(n1)N(C1CCCC1)CC(F)(F)C(=O)N2C] |
| 71 | 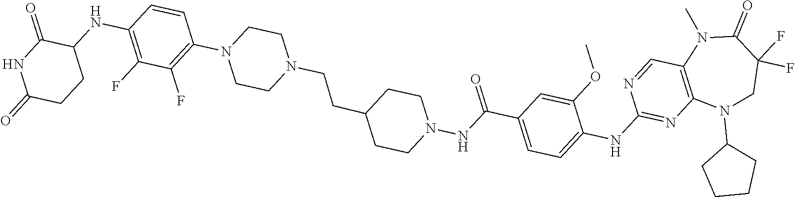 [SMILES: COc1cc(C(=O)NN2CCC(CCN3CCN(c4ccc(NC5CCC(=O)NC5=O)c(F)c4F)CC3)CC2)ccc1Nc1ncc2c(n1)N(C1CCCC1)CC(F)(F)C(=O)N2C] |
| 72 | 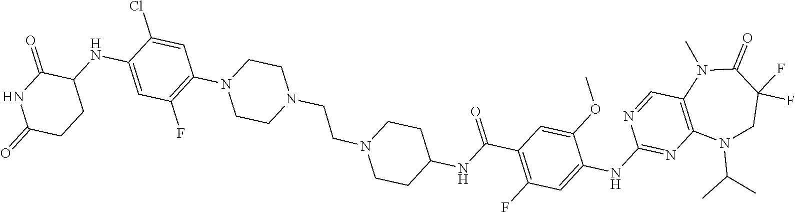 [SMILES: COc1cc(C(=O)NC2CCN(CCN3CCN(c4cc(Cl)c(NC5CCC(=O)NC5=O)cc4F)CC3)CC2)c(F)cc1Nc1ncc2c(n1)N(C(C)C)CC(F)(F)C(=O)N2C] |
| 73 | 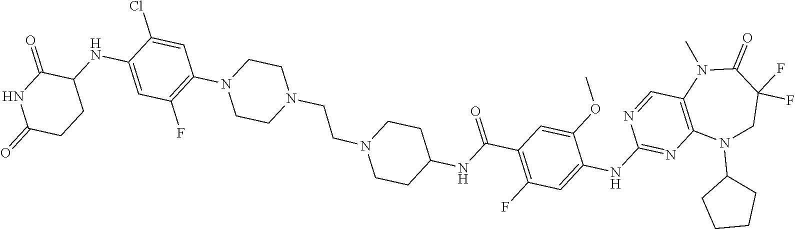 [SMILES: COc1cc(C(=O)NC2CCN(CCN3CCN(c4cc(Cl)c(NC5CCC(=O)NC5=O)cc4F)CC3)CC2)c(F)cc1Nc1ncc2c(n1)N(C1CCCC1)CC(F)(F)C(=O)N2C] |
| 74 | 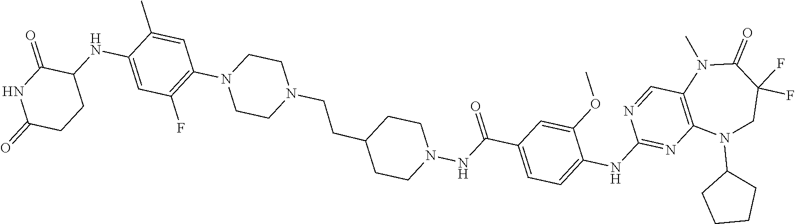 [SMILES: COc1cc(C(=O)NN2CCC(CCN3CCN(c4cc(C)c(NC5CCC(=O)NC5=O)cc4F)CC3)CC2)ccc1Nc1ncc2c(n1)N(C1CCCC1)CC(F)(F)C(=O)N2C] |
| 75 | 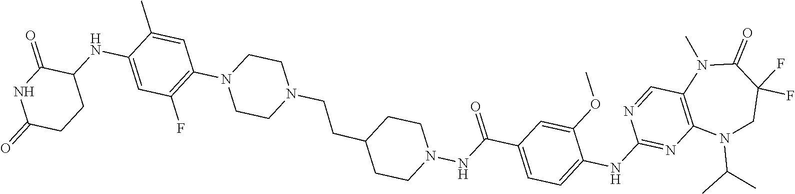 [SMILES: COc1cc(C(=O)NN2CCC(CCN3CCN(c4cc(C)c(NC5CCC(=O)NC5=O)cc4F)CC3)CC2)ccc1Nc1ncc2c(n1)N(C(C)C)CC(F)(F)C(=O)N2C] |
| 76 | 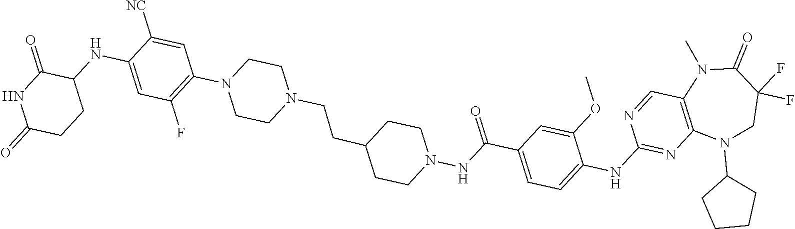 [SMILES: COc1cc(C(=O)NN2CCC(CCN3CCN(c4cc(C#N)c(NC5CCC(=O)NC5=O)cc4F)CC3)CC2)ccc1Nc1ncc2c(n1)N(C1CCCC1)CC(F)(F)C(=O)N2C] |
| 77 | 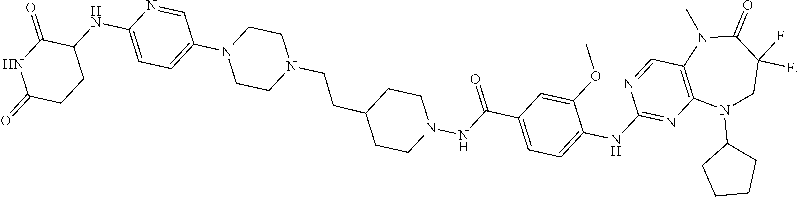 [SMILES: COc1cc(C(=O)NN2CCC(CCN3CCN(c4ccc(NC5CCC(=O)NC5=O)nc4)CC3)CC2)ccc1Nc1ncc2c(n1)N(C1CCCC1)CC(F)(F)C(=O)N2C] |

9. A pharmaceutical composition comprising the compound, the stereoisomer thereof or the pharmaceutically acceptable salt thereof according to claim 1.

10. A pharmaceutical composition for preventing or treating cancer, benign tumor or neurological disorder comprising the compound, the stereoisomer thereof or the pharmaceutically acceptable salt thereof according to claim 1.

11. A method for treating or preventing cancer, benign tumor or neurological disorder comprising administering to the subject in need thereof a therapeutically effective amount of the compound, the stereoisomer thereof or the pharmaceutically acceptable salt thereof according to claim 1.

12. The method for treating or preventing cancer, benign tumor or neurological disorder according to claim 11,
wherein the compound, the stereoisomer thereof or the pharmaceutically acceptable salt thereof induces degradation for PLK1 protein.

13. Use of the compound, the stereoisomer thereof or the pharmaceutically acceptable salt thereof according to claim 1.

14. Use of the compound, the stereoisomer thereof or the pharmaceutically acceptable salt thereof according to claim 1 for preparing a medicament for use in treating or preventing cancer, benign tumor or neurological disorder.
